(19) United States
(12) Patent Application Publication
(10) Pub. No.: US 2024/0408093 A1
(43) Pub. Date: Dec. 12, 2024

(54) HETEROBIFUNCTIONAL COMPOUNDS AND THEIR USE IN TREATING DISEASE

(71) Applicant: HALDA THERAPEUTICS OPCO, INC., New Haven, CT (US)

(72) Inventors: Samuel W. GERRITZ, Guilford, CT (US); Katherine J. KAYSER-BRICKER, Branford, CT (US); Taavi NEKLESA, Orange, CT (US); David E. PULEO, Fairfield, CT (US); James John MOUSSEAU, Salem, CT (US); Nilesh ZAWARE, Hamden, CT (US); Kyle J. EASTMAN, Killingworth, CT (US)

(21) Appl. No.: 18/696,145

(22) PCT Filed: Oct. 4, 2022
(86) PCT No.: PCT/US2022/045637

§ 371 Date: Mar. 27, 2024

(51) Int. Cl. A61K 31/517; A61K 31/4412; A61K 31/506; A61K 31/52; A61K 31/53; C07D 209/30; C07D 231/18; C07D 241/40; C07D 333/34; C07D 401/04; C07D 401/14; C07D 409/14; C07D 417/14; C07D 487/04; C07D 495/04

(52) U.S. Cl. CPC A61K 31/517; A61K 31/4412; A61K 31/506; A61K 31/52; A61K 31/53; C07D 209/30; C07D 231/18; C07D 241/40; C07D 333/34; C07D 401/04; C07D 401/14; C07D 409/14; C07D 417/14; C07D 487/04; C07D 495/04

(57) ABSTRACT

The invention provides heterobifunctional compounds comprising an effector protein binding moiety selected from GSPT1, Cyclin K, RBM23, RBM39, IKZF1, IKZF3, PLK1, CDK4 or CK1alpha which is linked to a moiety that binds to a target protein selected from KRAS, HER2, EGFR, androgen receptor protein, estrogen receptor protein, ALK, IDH1, FLT3, FGFR1, FGFR4, FGFR2, FGFR3, ERK1, ERK2, FGR, HER3 or HER4. Pharmaceutical compositions and their use in treating disease, such as cancer, are also disclosed.

### CROSS-REFERENCE TO RELATED APPLICATION

This application claims priority to U.S. provisional patent application No. 63/251,712, filed on Oct. 4, 2021, the contents of which are hereby incorporated by reference in their entirety.

### FIELD OF THE INVENTION

The invention provides heterobifunctional compounds, pharmaceutical compositions, and their use in protein degradation and treating disease, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. The incidence of prostate cancer increases with age, and with increasing longevity of human subjects, there continues to be a corresponding rise in the number of patients suffering from prostate cancer. Breast cancer is one of the most common cancers among women and is a leading cause of death for women between ages 50-55. Lung cancer is a leading cause of death among cancer patients, where over 85% of lung cancers are non-small cell lung cancer (NSCLC). Many lung cancers are attributed to tobacco smoking. Current treatment options for these cancers are not effective for all patients and/or can have substantial adverse side effects.

New therapies are needed to address this unmet need in cancer therapy. In particular, new therapies are needed that achieve an anti-cancer effect through a different mechanism than commonly available therapies. Exemplary mechanisms for common anti-cancer therapies include (a) alkylation of DNA which limits ability of the cell to reproduce, (b) topoisomerase inhibition, in which the therapeutic agent inhibits the activity of a topoisomerases thereby limiting separation of strands of DNA, and (c) mitotic inhibition, where the therapeutic agent reduces ability of the cell to divide. New therapies that achieve an anti-cancer effect through a different mechanism present an opportunity to treat cancers more effectively and/or to treat cancers that have become resistance to currently available medicines.

The present invention addresses the foregoing needs and provides other related advantages.

### SUMMARY

The invention provides heterobifunctional compounds, pharmaceutical compositions, and their use in protein degradation and treating disease, such as cancer. In particular, one aspect of the invention provides a collection of heterobifunctional compounds, such as a compound represented by Formula I:

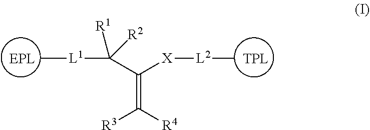

or a pharmaceutically acceptable salt thereof, where the variables are as defined in the detailed description. Further description of additional collections of related compounds are described in the detailed description. The compounds may be part of a pharmaceutical composition comprising a pharmaceutically acceptable carrier.

Another aspect of the invention provides a method of treating cancer. The method comprises administering to a patient in need thereof a therapeutically effective amount of a compound described herein, such as a compound of Formula I, to treat the cancer.

Another aspect of the invention provides a method of causing death of a cancer cell. The method comprises contacting a cancer cell with an effective amount of a compound described herein, such as a compound of Formula I, to cause death of the cancer cell.

Another aspect of the invention provides a method of degrading an effector protein in a cell. The method comprises administering to the cell an effective amount of a compound described herein, such as a compound of Formula I, resulting in degradation of the effector protein in the cell, wherein the effector protein is GSPT1, Cyclin K, RBM23, RBM39, IKZF1, IKZF3, PLK1, CDK4, or CK1alpha.

### DETAILED DESCRIPTION

The invention provides heterobifunctional compounds, pharmaceutical compositions, and their use in protein degradation and treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

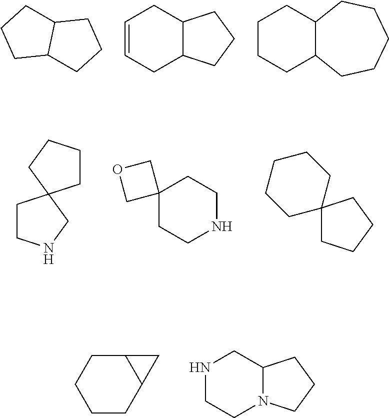

Exemplary bridged bicyclics include:

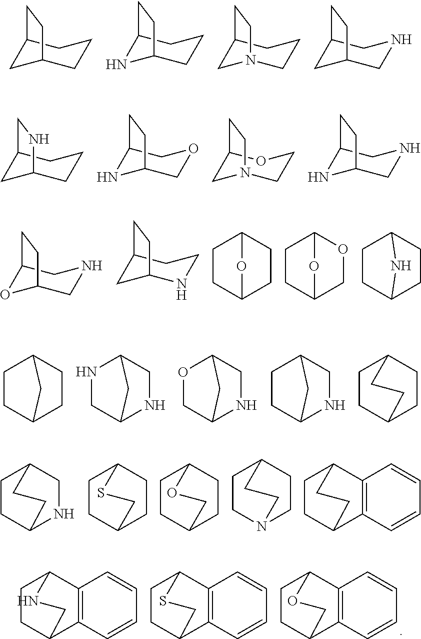

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)-group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “haloaryl” refers to an aryl group that is substituted with at least one halogen. Exemplary haloaryl groups include chlorophenyl (e.g., 3-chlorophenyl, 4-chlorophenyl), fluorophenyl, and the like. The term “phenylene” refers to a bivalent phenyl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 Tc electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “haloheteroaryl” refers to a heteroaryl group that is substituted with at least one halogen. Exemplary haloheteroaryl groups include chloropyridine, fluoropyridine, chloropyrazole, fluoropyrazole, and the like. The term “heteroarylene” refers to a bivalent heteroaryl group. Similarly, the terms “pyrazolylene”, “imidazolylene”, and “pyrrolylene”, respectively refer to bivalent pyrazolyl, imidazolyl, and pyrrolyl groups. Similarly, the terms “pyridinylene” and “pyrimidinylene”, respectively refer to bivalent pyridinyl and pyrimidinyl groups.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclyl group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR⁶⁰²₂; —(CH₂)₀₋₄N(R^{∘})C(O)OR^{∘}; —N(R^{∘})N(R^{∘})C(O)R^{∘}; —N(R^{∘})N(R^{∘})C(O)NR⁶⁰²₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄C(O)R^{∘}; —OC(O)(CH₂)₀₋₄SR—, SC(S)SR^{∘}; —(CH₂)₀₋₄SC(O)R^{∘}; —(CH₂)₀₋₄C(O)NR⁶⁰²₂; —C(S)NR^{∘}₂; —C(S)SR^{∘}; —SC(S)SR^{∘}, —(CH₂)₀₋₄C(O)NR⁶⁰²₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄S(O)₂R^{∘}; —S(O)₂NR⁶⁰²₂; —S(O)(NR^{∘})R^{∘}; —S(O)₂N═C(NR⁶⁰²₂)₂; —(CH₂)₀₋₄S(O)R^{∘}; —N(R^{∘})S(O)₂NR⁶⁰²₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃—O—, or —S(C(R*₂))₂₋₃S—, or a divalent substituent bound to vicinal substitutable carbons of an “optionally substituted” group is —O(CR*₂)₂₋₃—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “chloroalkyl” refers to an alkyl group that is substituted with at least one chloro. The term “bromoalkyl” refers to an alkyl group that is substituted with at least one bromo. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-8 membered heteroalkyl refers to a heteroalkyl group having from 3 to 8 atoms selected from the group consisting of carbon and a heteroatom. The term “heteroalkylene” refers to a bivalent heteroalkyl group.

The terms “alkenyl” and “alkynyl” are art-recognized and refer to unsaturated aliphatic groups analogous in length and possible substitution to the alkyls described above, but that contain at least one double or triple bond respectively. The term “haloalkenyl” refers to an alkenyl group that is substituted with at least one halogen. The term “fluoroalkenyl” refers to an alkenyl group that is substituted with at least one fluoro. The term “nitroalkenyl” refers to an alkenyl group that is substituted with at least one nitro.

The term “carbocyclylene” refers to a bivalent cycloaliphatic group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “=O” substituent. For example, a cyclopentane substituted with an oxo group is cyclopentanone.

The term “amino” is art-recognized and refers to both unsubstituted and substituted amines, e.g., a moiety that may be represented by the general formulas:

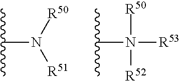

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8.

The term “amido” is art-recognized and refers to both unsubstituted and substituted amides, e.g., a moiety that may be represented by the general formulas:

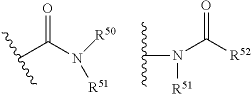

wherein R⁵⁰ and R⁵¹ each independently represent a hydrogen, an alkyl, an alkenyl, —(CH2)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8; and R⁵² is an alkyl, an alkenyl, or —(CH₂)ₘ—R⁶¹.

The symbol [symbol] indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Compounds

The invention provides heterobifunctional compounds. The compounds may be used in the pharmaceutical compositions and therapeutic methods described herein. Exemplary compounds are described in the following sections, along with exemplary procedures for making the compounds.

### Part A: Compounds of Formula I

One aspect of the invention provides a compound represented by Formula I:

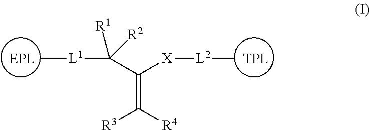

or a pharmaceutically acceptable salt thereof; wherein:
R¹, R², R³, and R⁴ are independently H, D, halo, or C₁₋₄ alkyl;
R⁵ is H or C₁₋₄ alkyl;
X is —C(O)— or —S(O)₂—;
EPL is a moiety that binds to an effector protein selected from GSPT1, Cyclin K, RBM23, RBM39, IKZF1, IKZF3, PLK1, CDK4, or CK1alpha;
TPL is a moiety that binds to a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4;
L¹ is a bond, **-linker-O—, or **-linker-N(R⁵)—, where ** is a point of attachment to EPL; and
L² is a bond or a linker; and
wherein L¹ is connected to a nitrogen atom of EPL when L¹ is a bond.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, R¹, R², R³, and R⁴ are independently H, D, halo, or C₁₋₄ alkyl.

In certain embodiments, R¹ is H, D, halo, or C₁₋₄ alkyl. In certain embodiments, R¹ is H or D. In certain embodiments, R¹ is H. In certain embodiments, R¹ halo. In certain embodiments, R¹ is C₁₋₄ alkyl. In certain embodiments, R² is H, D, halo, or C₁₋₄ alkyl. In certain embodiments, R² is H or D. In certain embodiments, R² is H. In certain embodiments, R² halo. In certain embodiments, R² is C₁₋₄ alkyl. In certain embodiments, R³ is H, D, halo, or C₁₋₄ alkyl. In certain embodiments, R³ is H or D. In certain embodiments, R³ is H. In certain embodiments, R³ halo. In certain embodiments, R³ is C₁₋₄ alkyl. In certain embodiments, R⁴ is H, D, halo, or C₁₋₄ alkyl. In certain embodiments, R⁴ is H or D. In certain embodiments, R⁴ is H. In certain embodiments, R⁴ halo. In certain embodiments, R⁴ is C₁₋₄ alkyl.

In certain embodiments, R¹ and R² are H. In certain embodiments, R³ and R⁴ are H.

In certain embodiments, R¹ is selected from those depicted in the compounds in any one of Tables 1, 2, 3, or 4 below. In certain embodiments, R² is selected from those depicted in the compounds in any one of Tables 1, 2, 3, or 4 below. In certain embodiments, R³ is selected from those depicted in the compounds in any one of Tables 1, 2, 3, or 4 below. In certain embodiments, R⁴ is selected from those depicted in the compounds in any one of Tables 1, 2, 3, or 4 below.

In certain embodiments, R¹ is selected from those depicted in the compounds in any one of Tables 1-A or 2-A below. In certain embodiments, R² is selected from those depicted in the compounds in any one of Tables 1-A or 2-A below. In certain embodiments, R³ is selected from those depicted in the compounds in any one of Tables 1-A or 2-A below. In certain embodiments, R⁴ is selected from those depicted in the compounds in any one of Tables 1-A or 2-A below.

As defined generally above, R⁵ is H or C₁₋₄ alkyl. In certain embodiments, R⁵ is H. In certain embodiments, R⁵ is C₁₋₄ alkyl. In certain embodiments, R⁵ is selected from those depicted in the compounds in any one of Tables 1, 2, 3, or 4 below. In certain embodiments, R⁵ is selected from those depicted in the compounds in any one of Tables 1-A or 2-A below.

As defined generally above, X is —C(O)— or —S(O)₂—. In certain embodiments, X is —C(O)—. In certain embodiments, X is —S(O)₂—. In certain embodiments, X is selected from those depicted in the compounds in any one of Tables 1, 2, 3, or 4 below. In certain embodiments, X is selected from those depicted in the compounds in any one of Tables 1-A or 2-A below.

As defined generally above, EPL is a moiety that binds to an effector protein selected from GSPT1, Cyclin K, RBM23, RBM39, IKZF1, IKZF3, PLK1, CDK4, or CK1alpha. In certain embodiments, the EPL is a moiety that binds to GSPT1.

In certain embodiments, the EPL has the following formula:

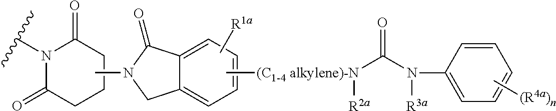

wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

In certain embodiments, the EPL has the following formula:

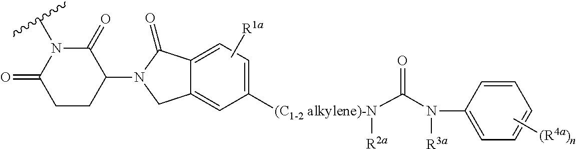

wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 1 or 2.

In certain embodiments, the EPL has the following formula:

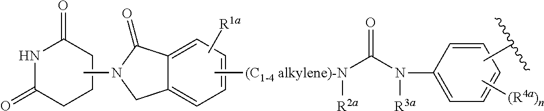

wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

In certain embodiments, the EPL has the following formula:

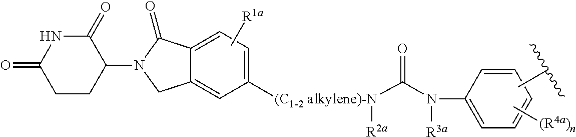

wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

In certain embodiments, the EPL is one of the following:

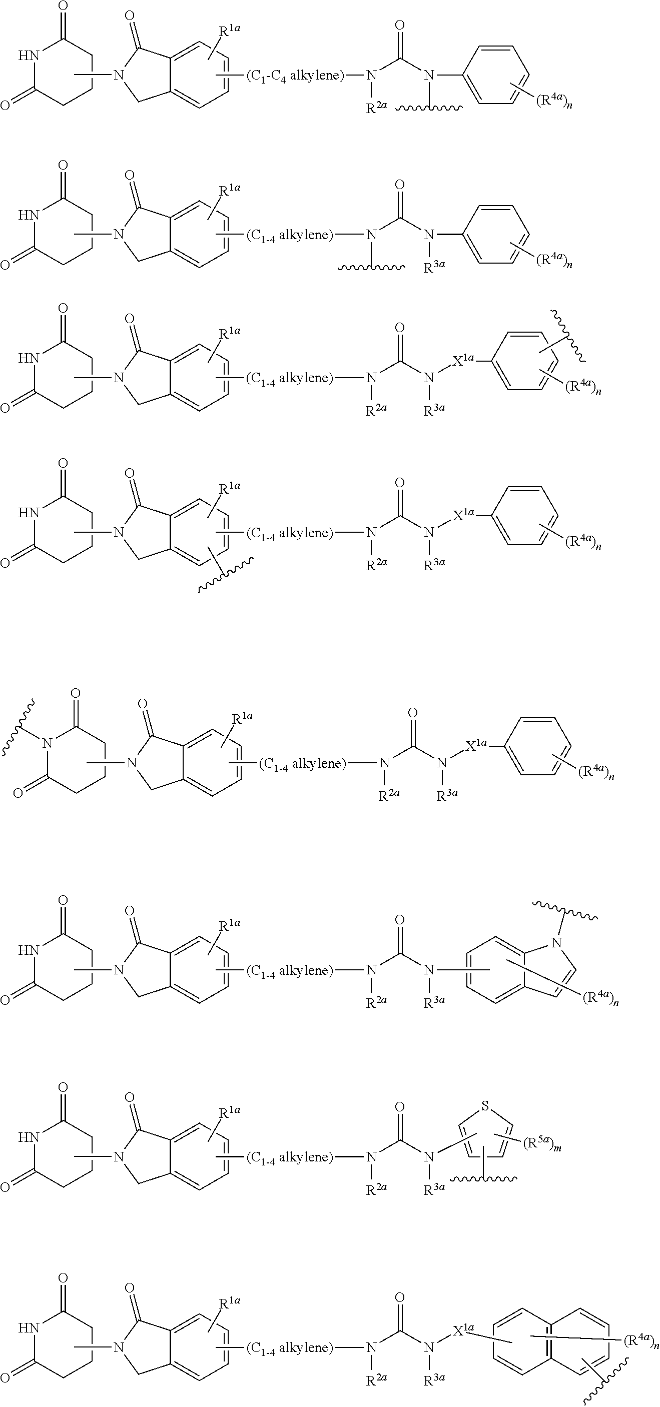

wherein:
   R^{1a} is hydrogen, halo, or C₁₋₄ alkyl;
   R^{2a} and R^{3a} each represent independently for each occurrence hydrogen or C₁₋₄ alkyl;
   R^{4a} and R^{5a} each represent independently for each occurrence halo, C₁₋₄ alkyl, C₁₋₄ haloalkyl, hydroxyl, nitro, or C₁₋₄ alkoxyl;
   X^{1a} is C₁₋₄ alkylene; and
   m is 0, 1, or 2;
   n is 0, 1, 2, or 3.

In certain embodiments, the EPL is

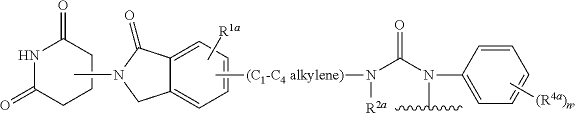

where the variables are as defined above. In certain embodiments, the EPL is

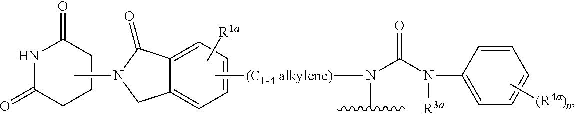

where the variables are as defined above. In certain embodiments, the EPL is

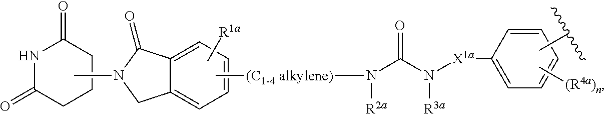

where the variables are as defined above. In certain embodiments, the EPL is

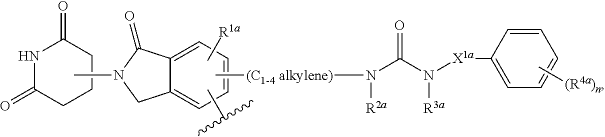

where the variables are as defined above.

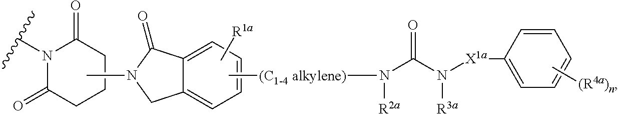

where the variables are as defined above. In certain embodiments, the EPL is

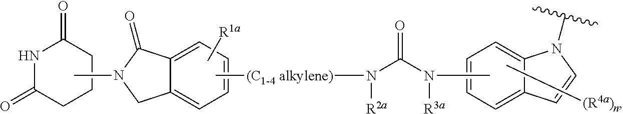

where the variables are as defined above. In certain embodiments, the EPL is

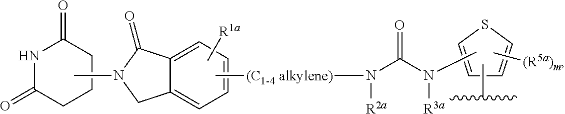

where the variables are as defined above. In certain embodiments, the EPL is

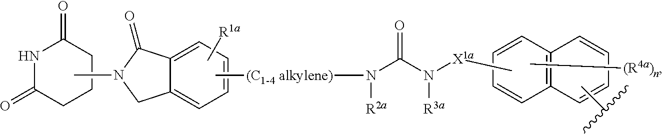

where the variables are as defined above.

In certain embodiments, R^{1a} is hydrogen. In certain embodiments, R^{1a} is halo. In certain embodiments, R^{1a} is C₁₋₄ alkyl. In certain embodiments, R^{2a} represents independently for each occurrence hydrogen or C₁₋₄ alkyl. In certain embodiments, R^{2a} is hydrogen. In certain embodiments, R^{2a} is C₁₋₄ alkyl.

In certain embodiments, R^{3a} represents independently for each occurrence hydrogen or C₁₋₄ alkyl. In certain embodiments, R^{3a} is hydrogen. In certain embodiments, R^{3a} is C₁₋₄ alkyl.

In certain embodiments, R^{4a} represents independently for each occurrence halo, C₁₋₄ alkyl, C₁₋₄ haloalkyl, hydroxyl, nitro, or C₁₋₄ alkoxyl. In certain embodiments, R^{4a} is halo. In certain embodiments, R^{4a} is C₁₋₄ alkyl. In certain embodiments, R^{4a} is C₁₋₄ haloalkyl. In certain embodiments, R^{4a} is hydroxyl. In certain embodiments, R^{4a} is nitro. In certain embodiments, R^{4a} is C₁₋₄ alkoxyl.

In certain embodiments, R^{5a} represents independently for each occurrence halo, C₁₋₄ alkyl, C₁₋₄ haloalkyl, hydroxyl, nitro, or C₁₋₄ alkoxyl. In certain embodiments, R^{5a} is halo. In certain embodiments, R^{5a} is C₁₋₄ alkyl. In certain embodiments, R^{5a} is C₁₋₄ haloalkyl. In certain embodiments, R^{5a} is hydroxyl. In certain embodiments, R^{5a} is nitro. In certain embodiments, R⁵ is C₁₋₄ alkoxyl.

In certain embodiments, X^{1a} is C₁₋₂ alkylene. In certain embodiments, X^{1a} is —CH₂—.

In certain embodiments, m is 0. In certain embodiments, m is 1. In certain embodiments, m is 2. In certain embodiments, n is 0. In certain embodiments, n is 1. In certain embodiments, n is 2. In certain embodiments, n is 3.

In certain embodiments, the EPL is one of the following:

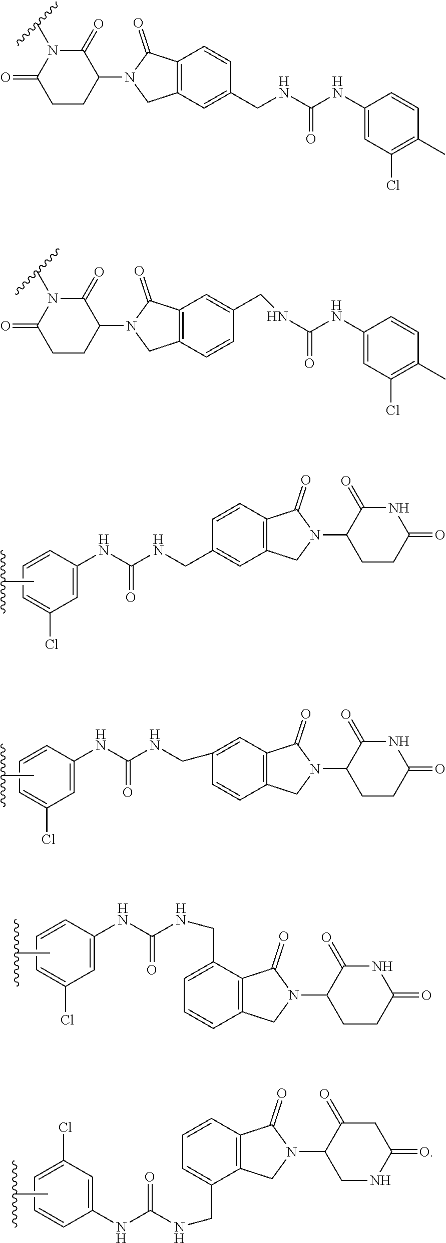

In certain embodiments, the EPL is one of the following:

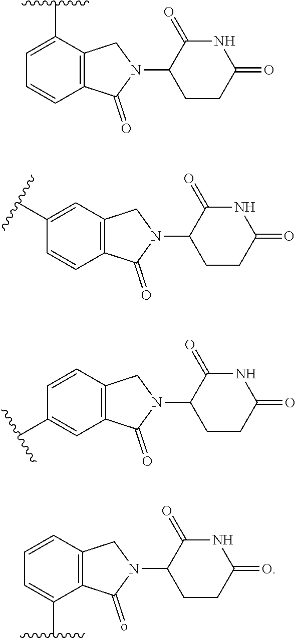

In certain embodiments, the EPL is one of the following:

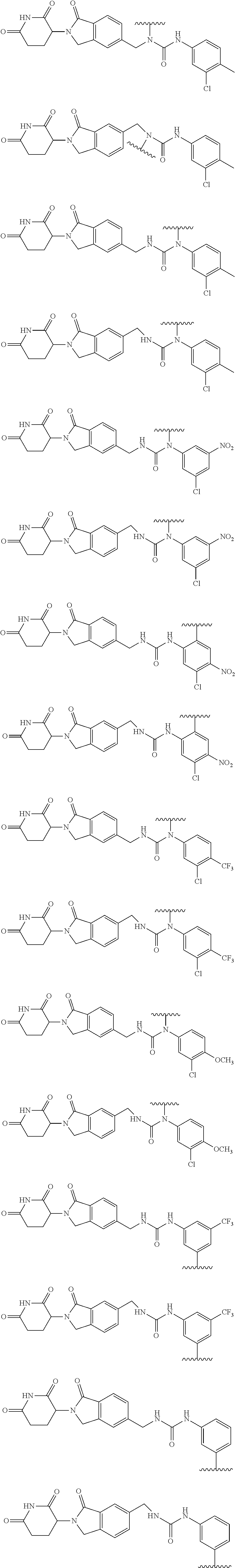

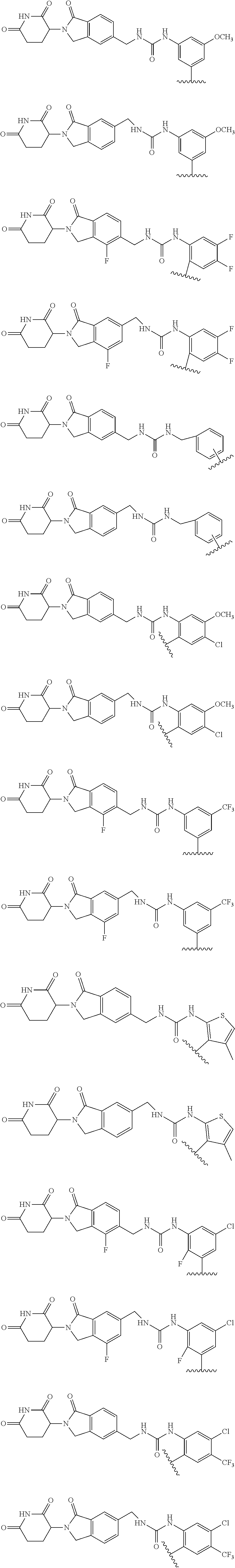

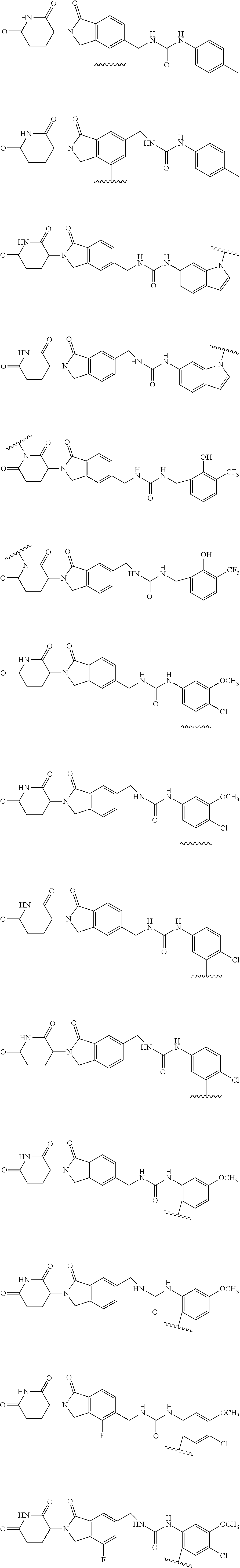

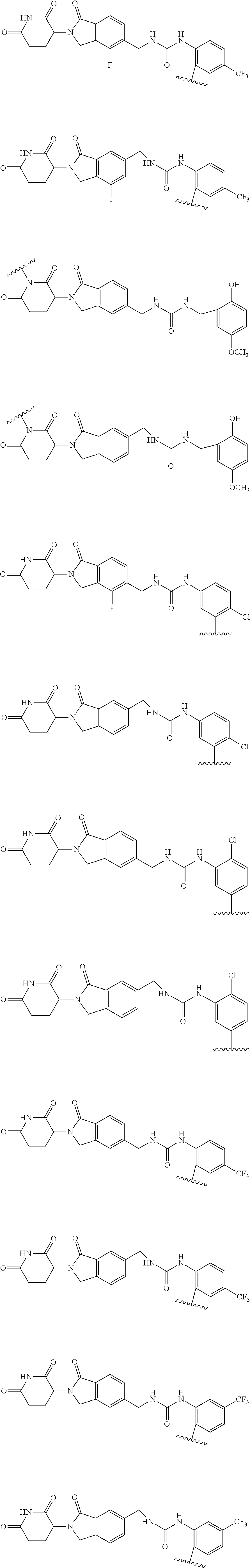

In certain embodiments, the EPL is a moiety that binds to Cyclin K.

In certain embodiments, the EPL is one of the following:

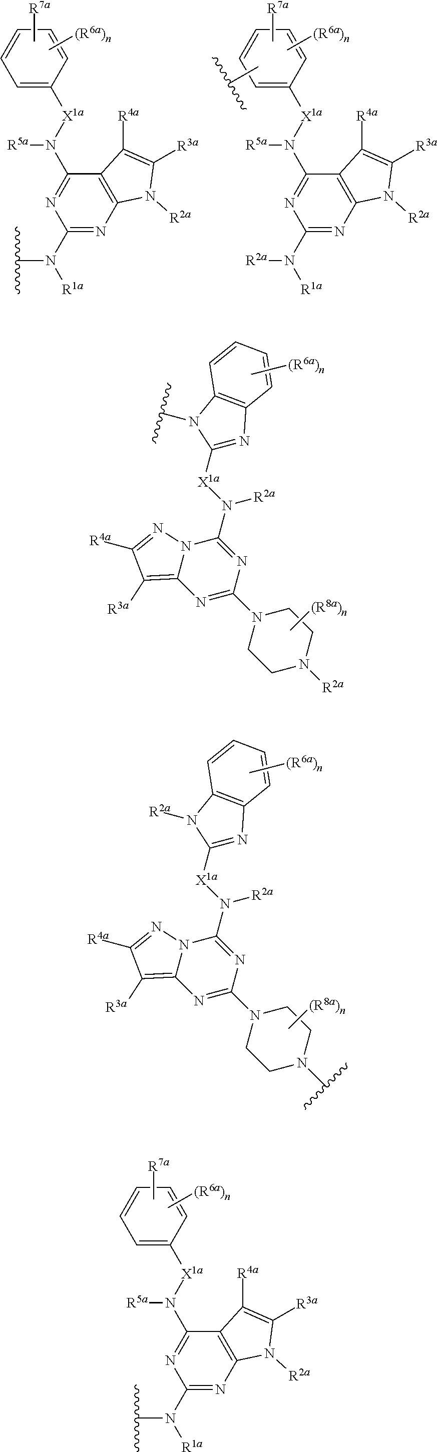

wherein:
R^{1a} is C₂₋₇ hydroxyalkyl;
R^{2a} and R^{5a} each represent independently for each occurrence hydrogen, C₁₋₄ alkyl, or C₃₋₆ cycloalkyl;
R^{3a}, R^{4a}, R^{6a}, and R^{8a} each represent independently for each occurrence hydrogen, C₁₋₄ alkyl, C₁₋₄ haloalkyl, C₃₋₆ cycloalkyl, or halo;
R^{7a} is a 5-6 membered heteroaryl containing 1, 2, or 3 heteroatoms independently selected from nitrogen, oxygen, and sulfur, wherein the heteroaryl is substituted with 0, 1, or 2 substituents independently selected from C₁₋₄ alkyl, C₁₋₄ haloalkyl, C₃₋₆ cycloalkyl, or halo;
X^{1a} is C₁₋₄ alkylene; and
n represents independently for each occurrence 0, 1, or 2.

In certain embodiments, the EPL is

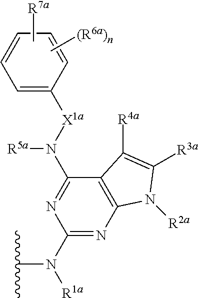

where the variables are as defined above. In certain embodiments, the EPL is

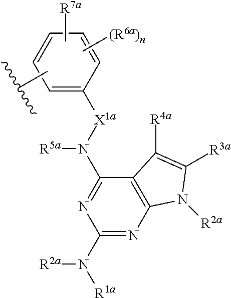

where the variables are as defined above. In certain embodiments, the EPL is

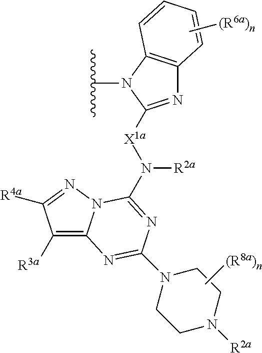

here the variables are as defined above.

In certain embodiments, the EPL is

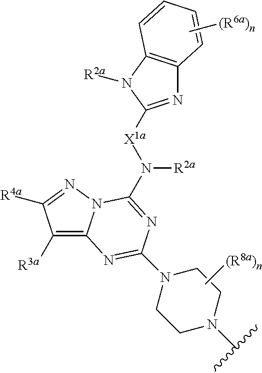

where the variables are as defined above. In certain embodiments, the EPL is

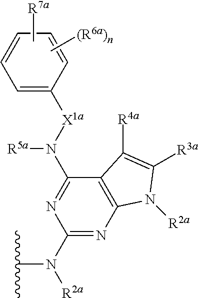

where the variables are as defined above.

In certain embodiments, R^{1a} is C₃₋₄ hydroxyalkyl.

In certain embodiments, R^{2a} represents independently for each occurrence hydrogen, C₁₋₄ alkyl, or C₃₋₆ cycloalkyl. In certain embodiments, R^{2a} is hydrogen. In certain embodiments, R^{2a} is C₁₋₄ alkyl. In certain embodiments, R^{2a} is C₃₋₆ cycloalkyl. In certain embodiments, R^{5a} is hydrogen. In certain embodiments, R^{5a} is C₁₋₄ alkyl. In certain embodiments, R^{5a} is C₃₋₆ cycloalkyl.

In certain embodiments, R^{3a} represents independently for each occurrence hydrogen, C₁₋₄ alkyl, C₁₋₄ haloalkyl, C₃₋₆ cycloalkyl, or halo. In certain embodiments, R^{3a} is hydrogen. In certain embodiments, R^{3a} is C₁₋₄ alkyl. In certain embodiments, R^{3a} is C₁₋₄ haloalkyl. In certain embodiments, R^{3a} is C₃₋₆ cycloalkyl. In certain embodiments, R^{3a} is halo.

In certain embodiments, R^{4a} represents independently for each occurrence hydrogen, C₁₋₄ alkyl, C₁₋₄ haloalkyl, C₃₋₆ cycloalkyl, or halo. In certain embodiments, R^{4a} is hydrogen. In certain embodiments, R^{4a} is C₁₋₄ alkyl. In certain embodiments, R^{4a} is C₁₋₄ haloalkyl. In certain embodiments, R^{4a} is C₃₋₆ cycloalkyl. In certain embodiments, R^{4a} is halo.

In certain embodiments, R^{6a} represents independently for each occurrence hydrogen, C₁₋₄ alkyl, C₁₋₄ haloalkyl, C₃₋₆ cycloalkyl, or halo. In certain embodiments, R^{6a} is hydrogen. In certain embodiments, R^{6a} is C₁₋₄ alkyl. In certain embodiments, R^{6a} is C₁₋₄ haloalkyl. In certain embodiments, R^{6a} is C₃₋₆ cycloalkyl. In certain embodiments, R^{6a} is halo.

In certain embodiments, R^{8a} represents independently for each occurrence hydrogen, C₁₋₄ alkyl, C₁₋₄ haloalkyl, C₃₋₆ cycloalkyl, or halo. In certain embodiments, R^{8a} is hydrogen. In certain embodiments, R^{8a} is C₁₋₄ alkyl. In certain embodiments, R^{8a} is C₁₋₄ haloalkyl. In certain embodiments, R^{8a} is C₃₋₆ cycloalkyl. In certain embodiments, R^{8a} is halo.

In certain embodiments, R^{7a} is a 6 membered heteroaryl containing 1 or 2 heteroatoms independently selected from nitrogen, oxygen, and sulfur, wherein the heteroaryl is substituted with 0, 1, or 2 substituents independently selected from C₁₋₄ alkyl, C₁₋₄ haloalkyl, C₃₋₆ cycloalkyl, or halo. In certain embodiments, R^{7a} is a 6 membered heteroaryl containing 1 or 2 heteroatoms independently selected from nitrogen, oxygen, and sulfur

In certain embodiments, X^{1a} is C₁₋₂ alkylene. In certain embodiments, X^{1a} is —CH₂—.

In certain embodiments, n is 0. In certain embodiments, n is 1. In certain embodiments, n is 2.

In certain embodiments, the EPL is one of the following:

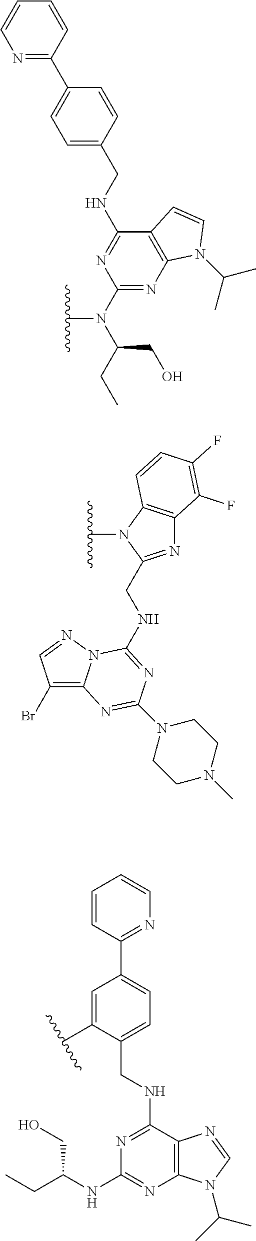

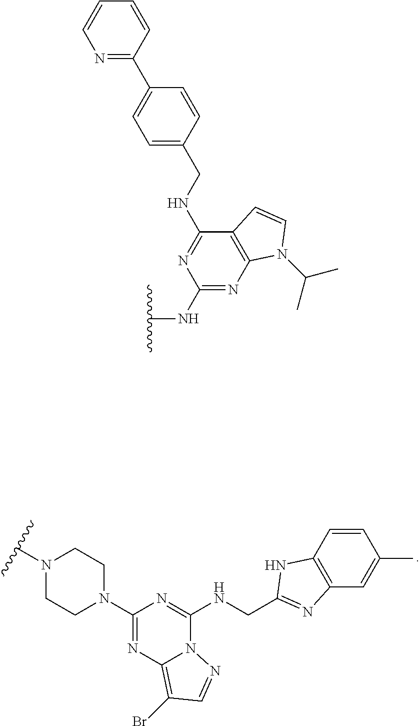 [SMILES: *Nc1nc(NCc2ccc(-c3ccccn3)cc2)c2ccn(C(C)C)c2n1 ; *N1CCN(c2nc(NCc3nc4cc(C)ccc4[nH]3)n3ncc(Br)c3n2)CC1]

In certain embodiments, the EPL is a moiety that binds to RBM23.

In certain embodiments, the EPL is a moiety that binds to RBM39.

In certain embodiments, the EPL is one of the following:

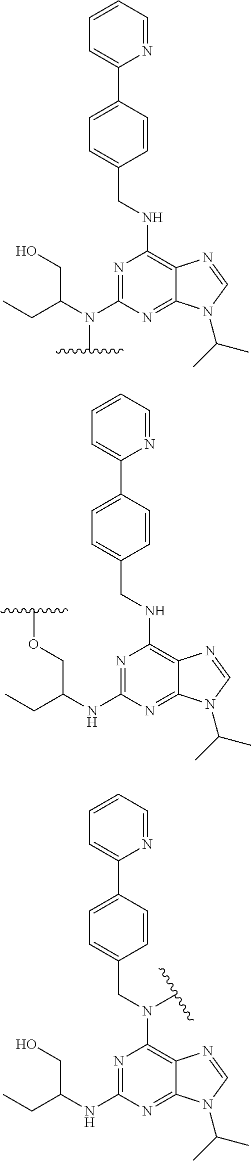

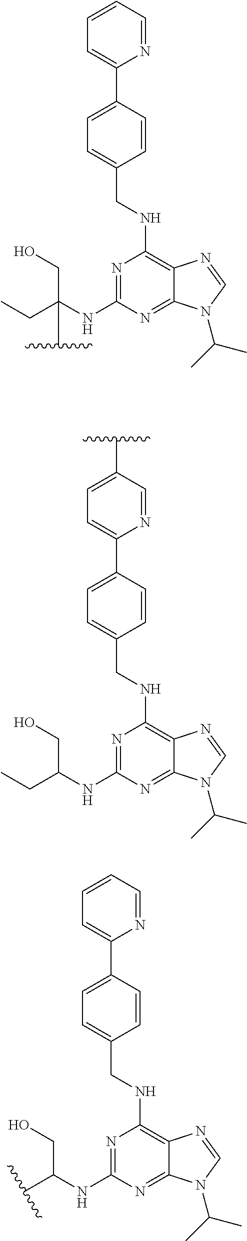

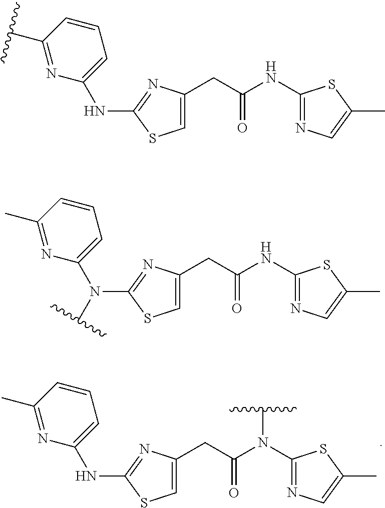

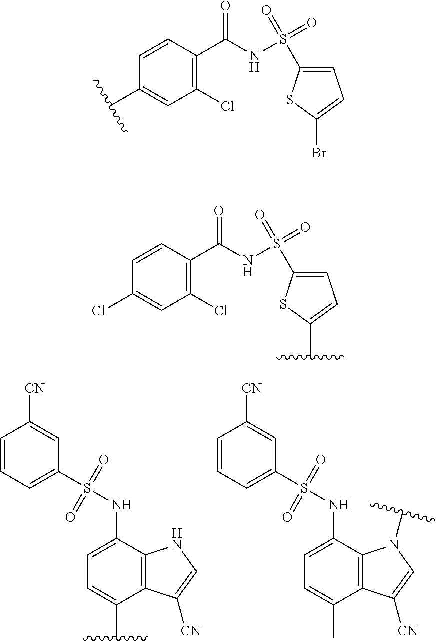 [SMILES: *c1ccc(C(=O)NS(=O)(=O)c2ccc(Br)s2)c(Cl)c1 ; *c1ccc(S(=O)(=O)NC(=O)c2ccc(Cl)cc2Cl)s1 ; *c1ccc(NS(=O)(=O)c2cccc(C#N)c2)c2[nH]cc(C#N)c12 ; *n1cc(C#N)c2c(C)ccc(NS(=O)(=O)c3cccc(C#N)c3)c21]

In certain embodiments, the EPL is one of the following:

In certain embodiments, the EPL is one of the following:

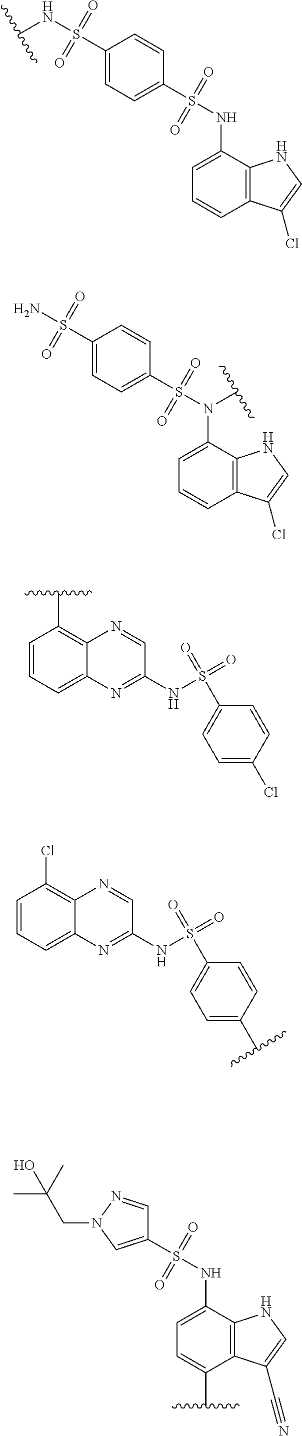 [SMILES: *NS(=O)(=O)c1ccc(S(=O)(=O)Nc2cccc3c(Cl)c[nH]c23)cc1 ; *N(c1cccc2c(Cl)c[nH]c12)S(=O)(=O)c1ccc(S(N)(=O)=O)cc1 ; *c1cccc2nc(NS(=O)(=O)c3ccc(Cl)cc3)cnc12 ; *c1ccc(S(=O)(=O)Nc2cnc3c(Cl)cccc3n2)cc1 ; *c1ccc(NS(=O)(=O)c2cnn(CC(C)(C)O)c2)c2[nH]cc(C#N)c12]

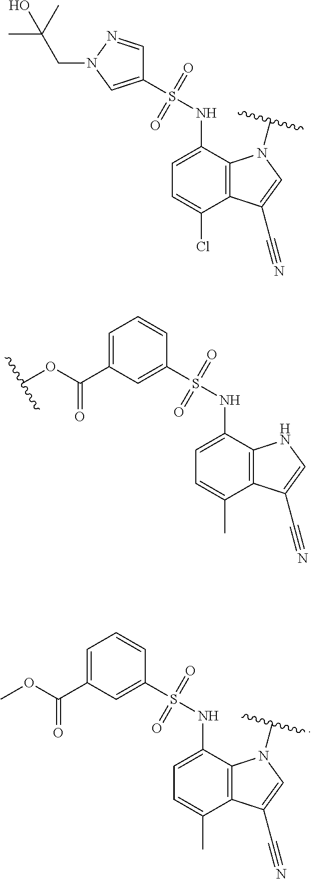 [SMILES: *n1cc(C#N)c2c(Cl)ccc(NS(=O)(=O)c3cnn(CC(C)(C)O)c3)c21 ; *OC(=O)c1cccc(S(=O)(=O)Nc2ccc(C)c3c(C#N)c[nH]c23)c1 ; *n1cc(C#N)c2c(C)ccc(NS(=O)(=O)c3cccc(C(=O)OC)c3)c21]

In certain embodiments, the EPL is a moiety that binds to an effector protein selected from IKZF1 or IKZF3. In certain embodiments, the EPL is a moiety that binds to IKZF1. In certain embodiments, the EPL is a moiety that binds to IKZF3.

In certain embodiments, the EPL is one of the following:

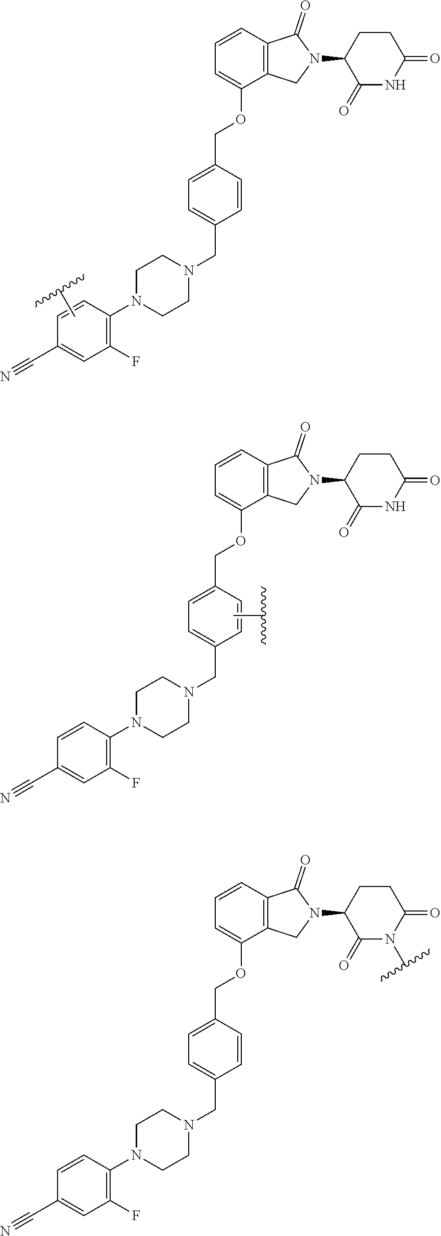

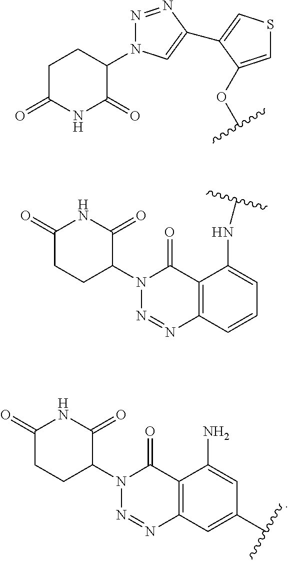 [SMILES: *Oc1cscc1-c1cn(C2CCC(=O)NC2=O)nn1 ; *Nc1cccc2nnn(C3CCC(=O)NC3=O)c(=O)c12 ; *c1cc(N)c2c(=O)n(C3CCC(=O)NC3=O)nnc2c1]

In certain embodiments, the EPL is one of the following:

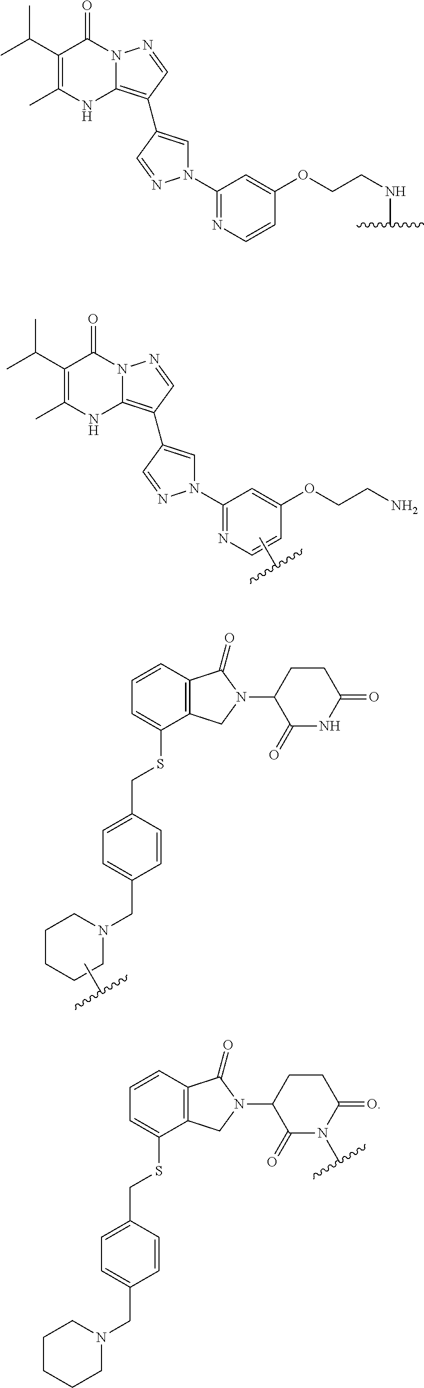

In certain embodiments, the EPL is a moiety that binds to an effector protein selected from PLK1, CDK4, or CK1alpha. In certain embodiments, the EPL is a moiety that binds to PLK1. In certain embodiments, the EPL is a moiety that binds to CDK4. In certain embodiments, the EPL is a moiety that binds to CK1alpha.

In certain embodiments, the EPL is one of the following:

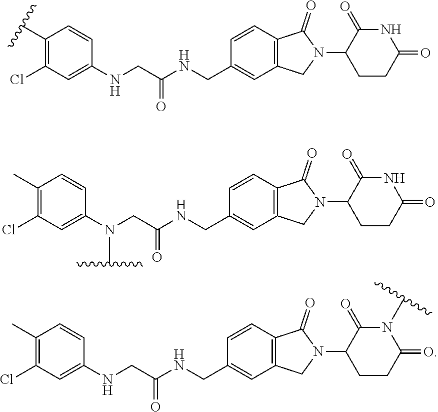 [SMILES: *c1ccc(NCC(=O)NCc2ccc3c(c2)CN(C2CCC(=O)NC2=O)C3=O)cc1Cl ; *N(CC(=O)NCc1ccc2c(c1)CN(C1CCC(=O)NC1=O)C2=O)c1ccc(C)c(Cl)c1 ; *N1C(=O)CCC(N2Cc3cc(CNC(=O)CNc4ccc(C)c(Cl)c4)ccc3C2=O)C1=O]

In certain embodiments, the EPL is selected from those depicted in the compounds in any one of Tables 1, 2, 3, or 4 below.

Additional exemplary EPL components are described in more detail below.

### A. Moiety for GSPT1

In certain embodiments, the EPL is a moiety that binds to Eukaryotic Peptide Chain Release Factor GTP-Binding Subunit ERF3A (GSPT1). Exemplary moieties that bind GSPT1 are reported in the literature, including:

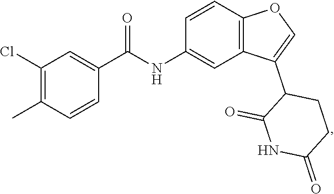 [SMILES: Cc1ccc(C(=O)Nc2ccc3occ(C4CCC(=O)NC4=O)c3c2)cc1Cl]

as described in Luo, Y. et al., in WO2021047627.

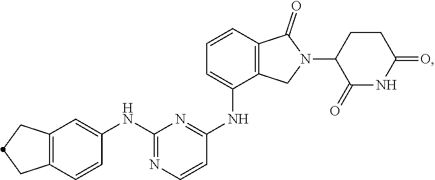

as described in Gray, N. et al., in WO2020006264.

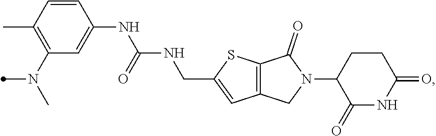

as described in Chan, K. et al., in US2020369679.

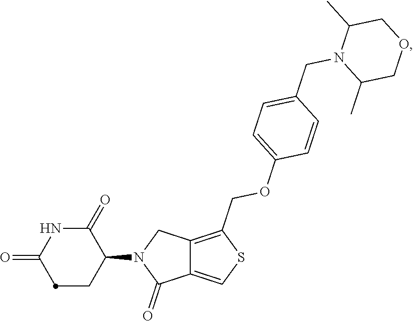

as described in Chan, K. et al., in WO2019241271.

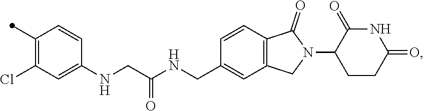

as described in Chan, K. et al., in WO2019241274.

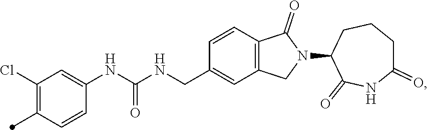

as described in Chan, K. et al., in WO2019241274.

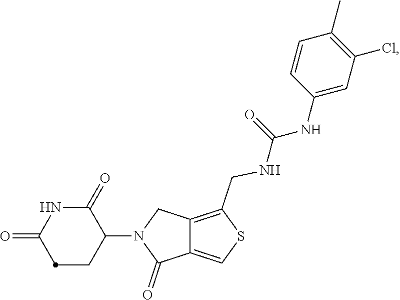

as described in Chan, K. et al., in US2018298027.

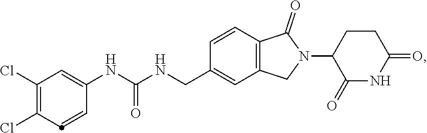

as described in Muller, G. et al., in US2009142297.

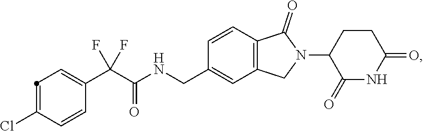

as described in Hansen, J. et al., in WO2016007848.

In certain embodiments, the EPL is a radical of one of the above compounds, which is attached to L¹ through a modifiable oxygen, nitrogen, or carbon atom.

In certain embodiments, the EPL is one of the following:

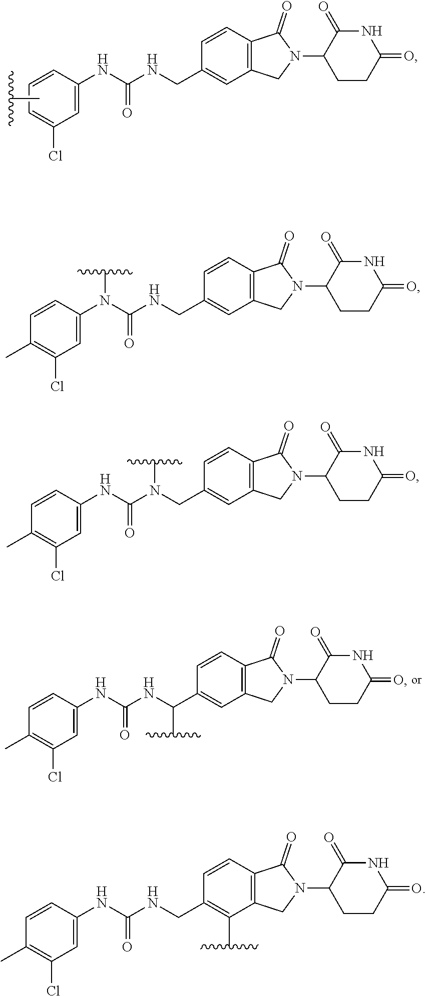

In certain embodiments, the EPL is

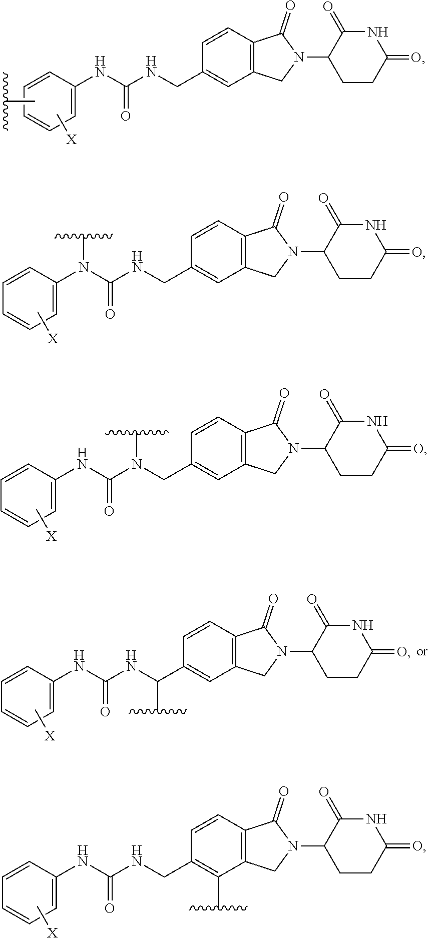

wherein X is H, D, halo, C₁₋₆ alkyl, amino, amido, amino(C₁₋₆ alkyl), C₁₋₆ alkoxy, or hydroxy.

In certain embodiments, the EPL is

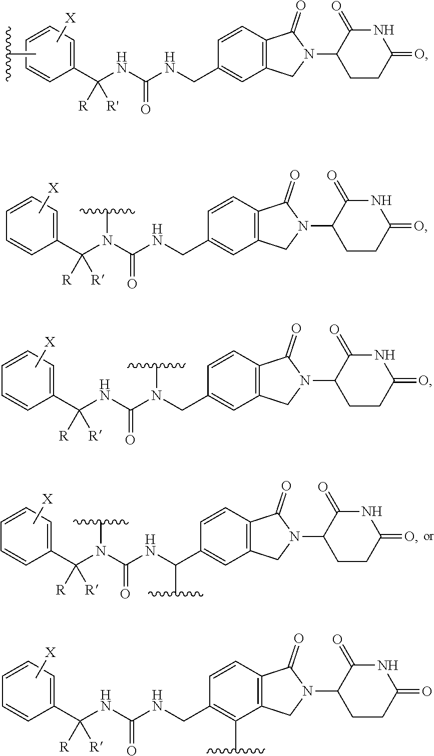

wherein X is H, D, halo, C₁₋₆ alkyl, amino, amido, amino(C₁₋₆ alkyl), C₁₋₆ alkoxy, or hydroxy; R is H, D, halo, C₁₋₆ alkyl, amino, amido, amino(C₁₋₆ alkyl), C₁₋₆ alkoxy, or hydroxy; and R′ is H, D, halo, C₁₋₆ alkyl, amino, amido, amino(C₁₋₆ alkyl), C₁₋₆ alkoxy, or hydroxy.

In certain embodiments, the EPL is

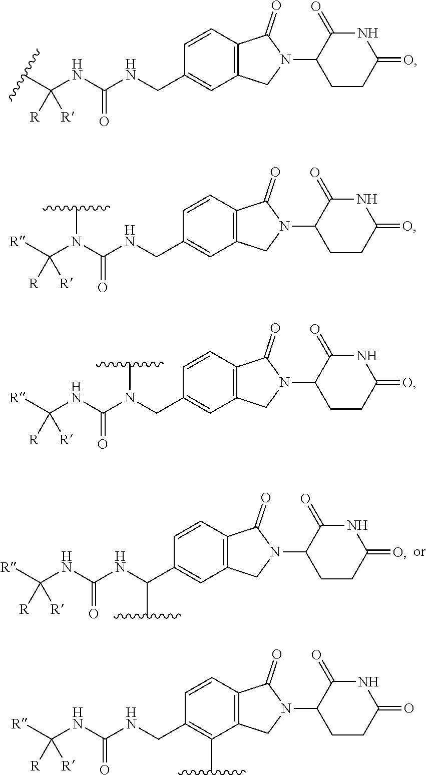

wherein R is H, D, halo, C₁₋₆ alkyl, amino, amido, aminoalkyl, alkoxy, or hydroxy; R′ is H, D, halo, C₁₋₆ alkyl, amino, amido, amino(C₁₋₆ alkyl), C₁₋₆ alkoxy, or hydroxy; and R″ is H, D, halo, C₁₋₆ alkyl, amino, amido, amnoalkyl, C₁₋₆ alkoxy, hydroxy, aryl, 3-10 membered heteroaryl, C₃₋₇ cycloalkyl, or 3-10 membered heterocyclyl.

In certain embodiments, the EPL is one of the following:

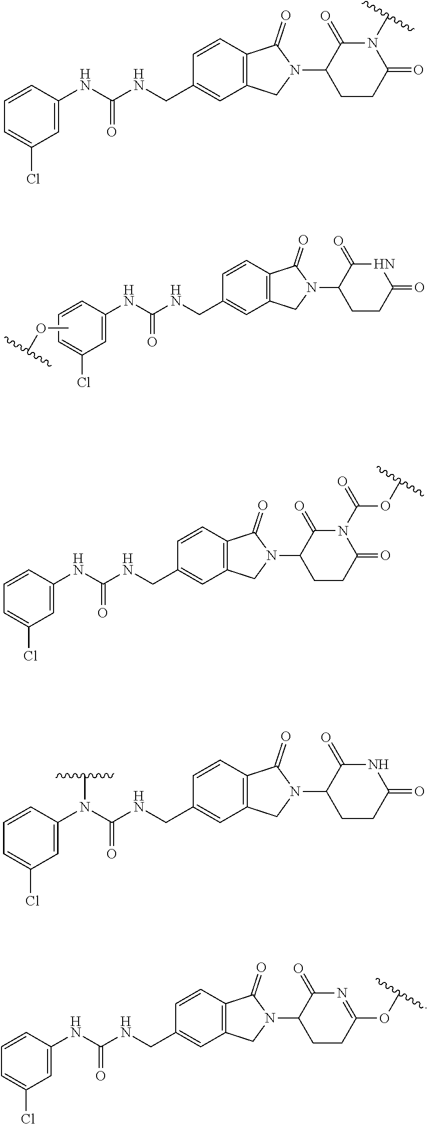

### B. Moiety for Cyclin K

In certain embodiments, the EPL is a moiety that binds to or degrades Cyclin K. Exemplary compounds that bind to and/or degrade Cyclin K are reported in the literature, including:

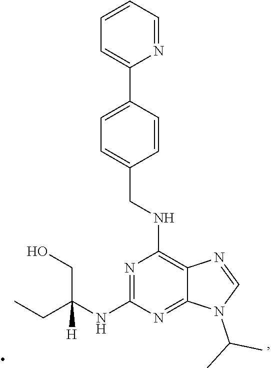 [SMILES: CC[C@H](CO)Nc1nc(NCc2ccc(-c3ccccn3)cc2)c2ncn(C(C)C)c2n1]

as described in Slabicki, M. et al., in Nature (London, United Kingdom) (2020), 585(7824): 293.

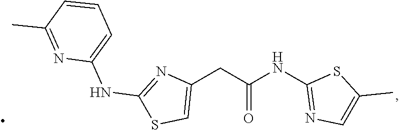

as described in Lv, L. et al., in eLife (2020), 9: e59994.

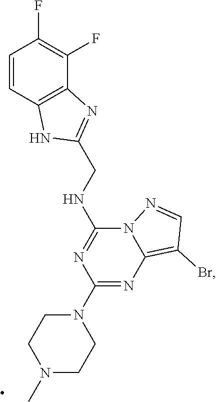 [SMILES: CN1CCN(c2nc(NCc3nc4c(F)c(F)ccc4[nH]3)n3ncc(Br)c3n2)CC1]

as described by Thede, K. et al., WO2021116178.

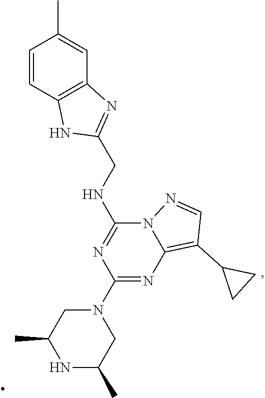 [SMILES: Cc1ccc2[nH]c(CNc3nc(N4C[C@@H](C)N[C@@H](C)C4)nc4c(C5CC5)cnn34)nc2c1]

as described by Thede, K. et al., WO2021116178.

In certain embodiments, the EPL is a radical of one of the above compounds, which is attached to L¹ through a modifiable oxygen, nitrogen, or carbon atom.

In certain embodiments, the EPL is one of the following:

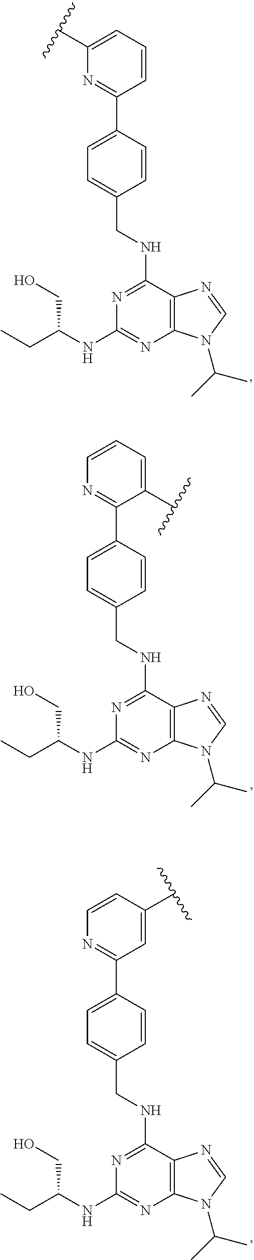

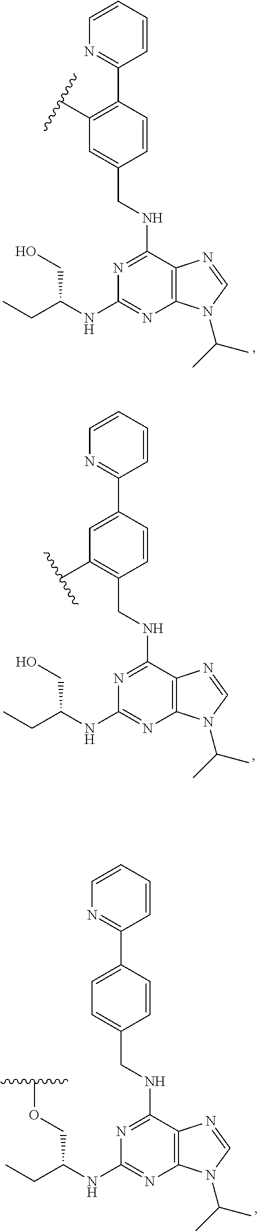

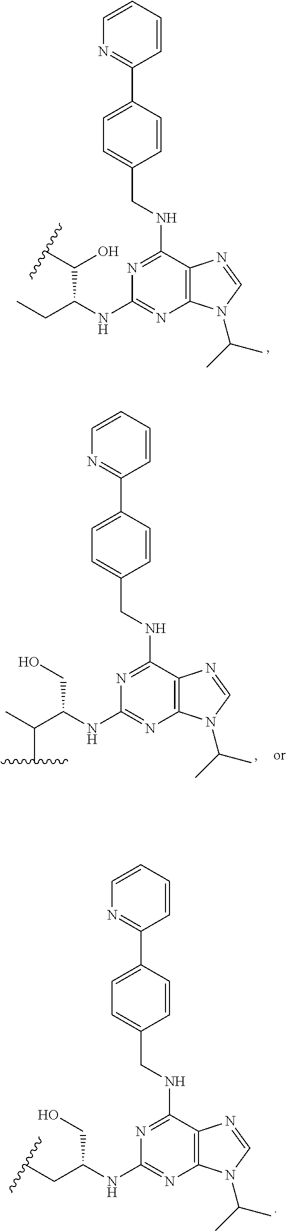

In certain embodiments, the EPL is one of the following:

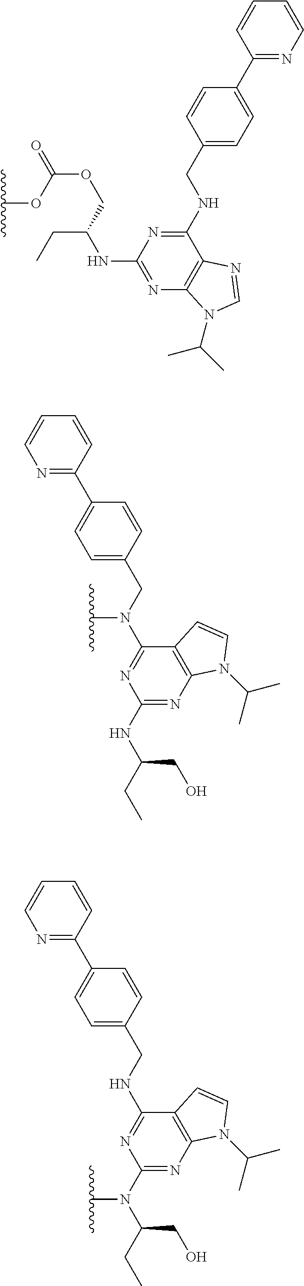 [SMILES: *OC(=O)OC[C@@H](CC)Nc1nc(NCc2ccc(-c3ccccn3)cc2)c2ncn(C(C)C)c2n1 ; *N(Cc1ccc(-c2ccccn2)cc1)c1nc(N[C@H](CC)CO)nc2c1ccn2C(C)C ; *N(c1nc(NCc2ccc(-c3ccccn3)cc2)c2ccn(C(C)C)c2n1)[C@H](CC)CO]

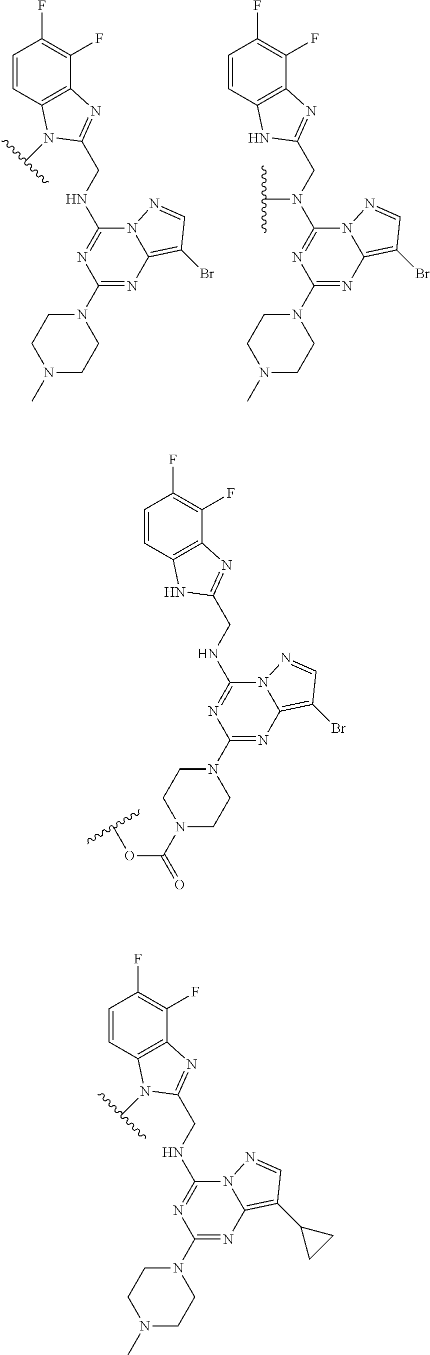 [SMILES: *n1c(CNc2nc(N3CCN(C)CC3)nc3c(Br)cnn23)nc2c(F)c(F)ccc21 ; *N(Cc1nc2c(F)c(F)ccc2[nH]1)c1nc(N2CCN(C)CC2)nc2c(Br)cnn12 ; *OC(=O)N1CCN(c2nc(NCc3nc4c(F)c(F)ccc4[nH]3)n3ncc(Br)c3n2)CC1 ; *n1c(CNc2nc(N3CCN(C)CC3)nc3c(C4CC4)cnn23)nc2c(F)c(F)ccc21]

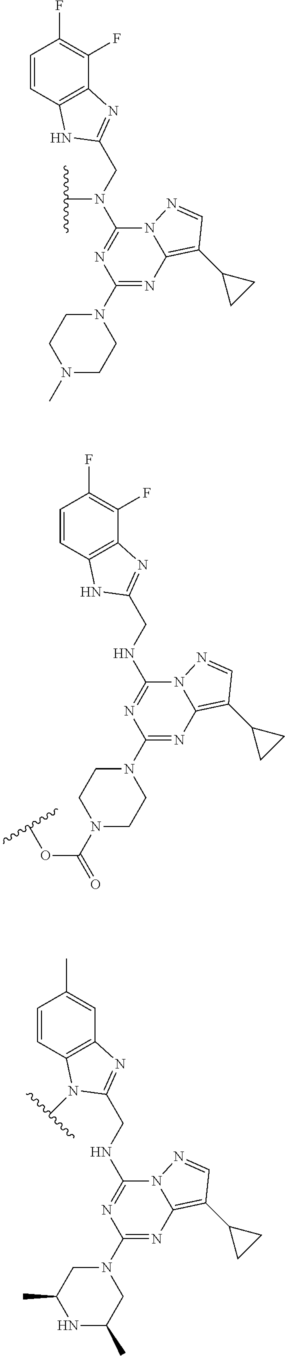 [SMILES: *N(Cc1nc2c(F)c(F)ccc2[nH]1)c1nc(N2CCN(C)CC2)nc2c(C3CC3)cnn12 ; *OC(=O)N1CCN(c2nc(NCc3nc4c(F)c(F)ccc4[nH]3)n3ncc(C4CC4)c3n2)CC1 ; *n1c(CNc2nc(N3C[C@@H](C)N[C@@H](C)C3)nc3c(C4CC4)cnn23)nc2cc(C)ccc21]

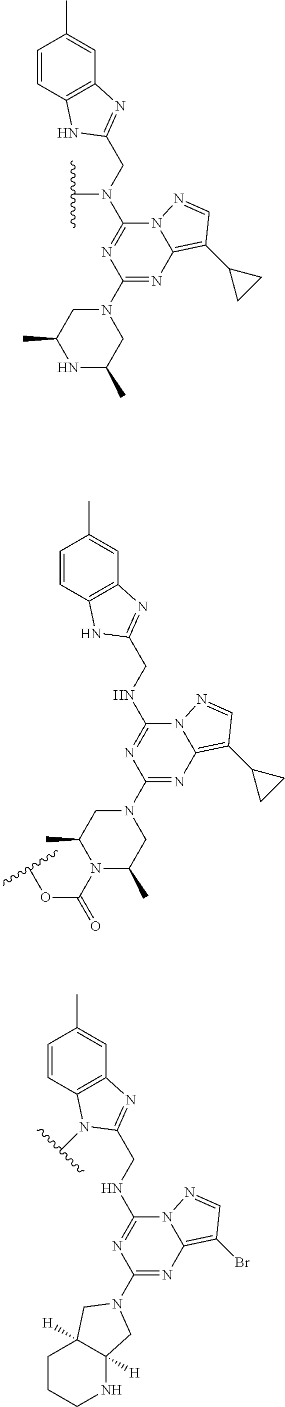 [SMILES: *N(Cc1nc2cc(C)ccc2[nH]1)c1nc(N2C[C@@H](C)N[C@@H](C)C2)nc2c(C3CC3)cnn12 ; *OC(=O)N1[C@H](C)CN(c2nc(NCc3nc4cc(C)ccc4[nH]3)n3ncc(C4CC4)c3n2)C[C@@H]1C ; *n1c(CNc2nc(N3C[C@@H]4CCCN[C@@H]4C3)nc3c(Br)cnn23)nc2cc(C)ccc21]

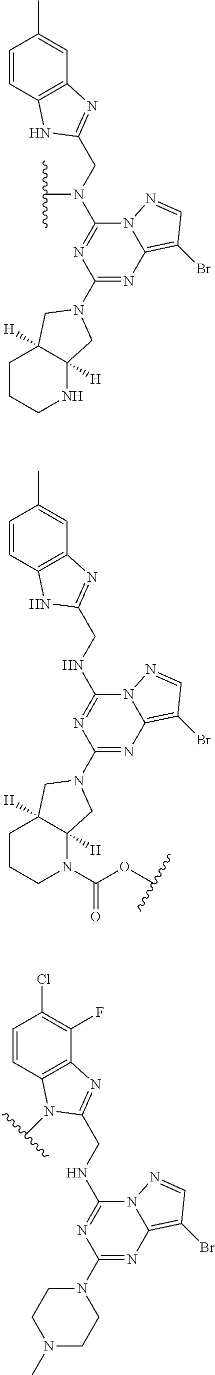 [SMILES: *N(Cc1nc2cc(C)ccc2[nH]1)c1nc(N2C[C@@H]3CCCN[C@@H]3C2)nc2c(Br)cnn12 ; *OC(=O)N1CCC[C@H]2CN(c3nc(NCc4nc5cc(C)ccc5[nH]4)n4ncc(Br)c4n3)C[C@H]21 ; *n1c(CNc2nc(N3CCN(C)CC3)nc3c(Br)cnn23)nc2c(F)c(Cl)ccc21]

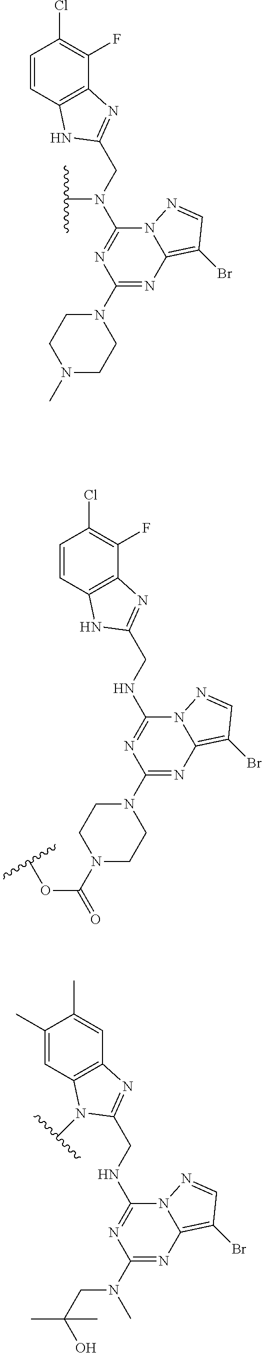 [SMILES: *N(Cc1nc2c(F)c(Cl)ccc2[nH]1)c1nc(N2CCN(C)CC2)nc2c(Br)cnn12 ; *OC(=O)N1CCN(c2nc(NCc3nc4c(F)c(Cl)ccc4[nH]3)n3ncc(Br)c3n2)CC1 ; *n1c(CNc2nc(N(C)CC(C)(C)O)nc3c(Br)cnn23)nc2cc(C)c(C)cc21]

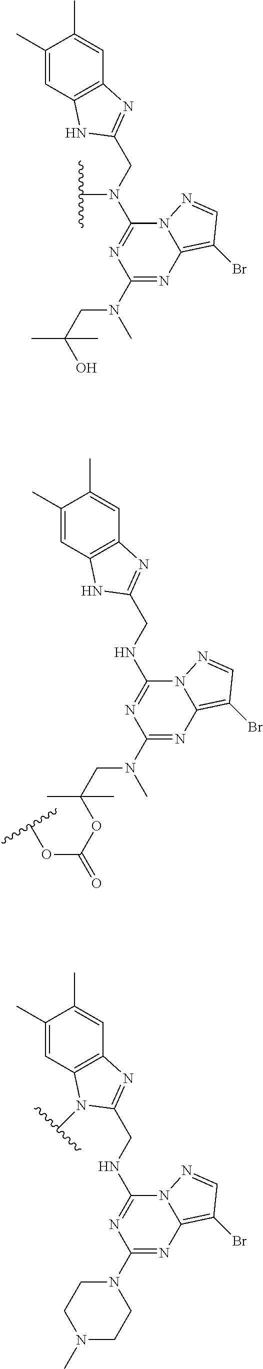 [SMILES: *N(Cc1nc2cc(C)c(C)cc2[nH]1)c1nc(N(C)CC(C)(C)O)nc2c(Br)cnn12 ; *OC(=O)OC(C)(C)CN(C)c1nc(NCc2nc3cc(C)c(C)cc3[nH]2)n2ncc(Br)c2n1 ; *n1c(CNc2nc(N3CCN(C)CC3)nc3c(Br)cnn23)nc2cc(C)c(C)cc21]

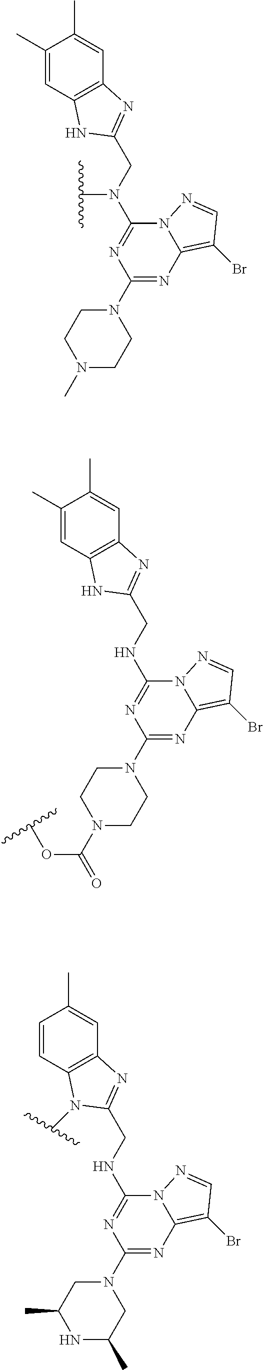 [SMILES: *N(Cc1nc2cc(C)c(C)cc2[nH]1)c1nc(N2CCN(C)CC2)nc2c(Br)cnn12 ; *OC(=O)N1CCN(c2nc(NCc3nc4cc(C)c(C)cc4[nH]3)n3ncc(Br)c3n2)CC1 ; *n1c(CNc2nc(N3C[C@@H](C)N[C@@H](C)C3)nc3c(Br)cnn23)nc2cc(C)ccc21]

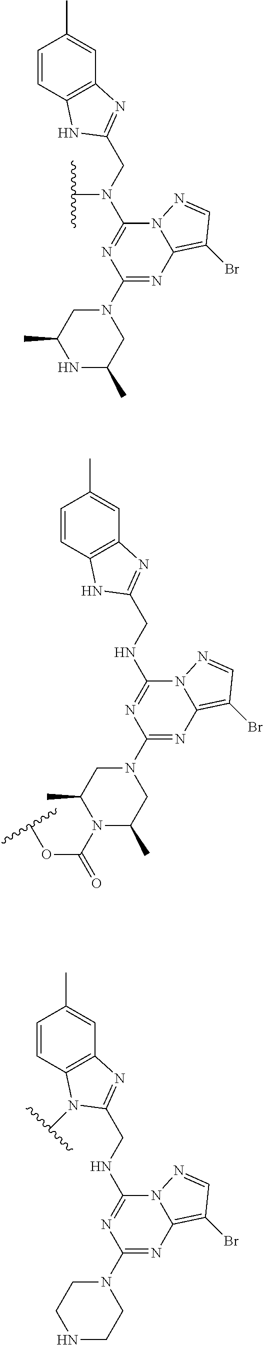 [SMILES: *N(Cc1nc2cc(C)ccc2[nH]1)c1nc(N2C[C@@H](C)N[C@@H](C)C2)nc2c(Br)cnn12 ; *OC(=O)N1[C@H](C)CN(c2nc(NCc3nc4cc(C)ccc4[nH]3)n3ncc(Br)c3n2)C[C@@H]1C ; *n1c(CNc2nc(N3CCNCC3)nc3c(Br)cnn23)nc2cc(C)ccc21]

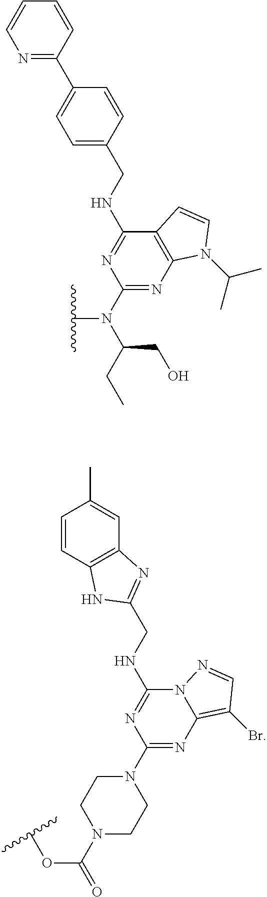 [SMILES: *N(c1nc(NCc2ccc(-c3ccccn3)cc2)c2ccn(C(C)C)c2n1)[C@H](CC)CO ; *OC(=O)N1CCN(c2nc(NCc3nc4cc(C)ccc4[nH]3)n3ncc(Br)c3n2)CC1]

### C. Moiety for RBM39

In certain embodiments, the EPL is a moiety that binds to or degrades RBM39. Exemplary compounds that bind to and/or degrade RBM39 are reported in the literature, including:

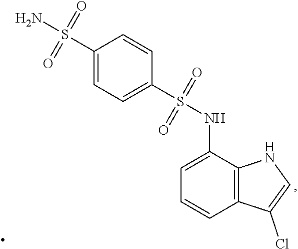 [SMILES: NS(=O)(=O)c1ccc(S(=O)(=O)Nc2cccc3c(Cl)c[nH]c23)cc1]

as described in Han, T. et al., Science (2017), 356(6336): 3755.

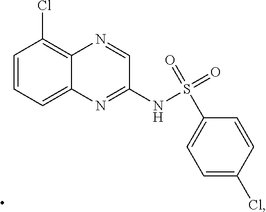 [SMILES: O=S(=O)(Nc1cnc2c(Cl)cccc2n1)c1ccc(Cl)cc1]

as described in Han, T. et al., Science (2017), 356(6336): 3755.

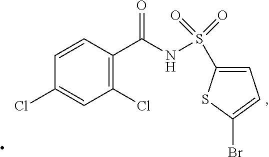 [SMILES: O=C(NS(=O)(=O)c1ccc(Br)s1)c1ccc(Cl)cc1Cl]

as described in Han, T. et al., Science (2017), 356(6336): 3755.

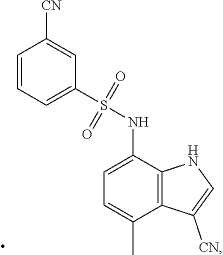 [SMILES: Cc1ccc(NS(=O)(=O)c2cccc(C#N)c2)c2[nH]cc(C#N)c12]

as described in Uehara, T. et al., Nat. Chem. Bio (2017), 13:675.

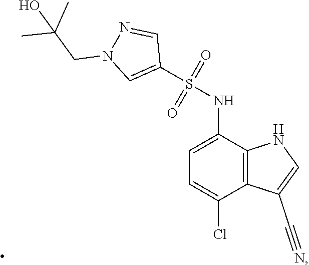 [SMILES: CC(C)(O)Cn1cc(S(=O)(=O)Nc2ccc(Cl)c3c(C#N)c[nH]c23)cn1]

as described in Estrada, M. et al., WO2020210139.

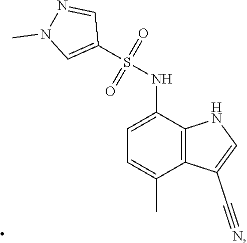 [SMILES: Cc1ccc(NS(=O)(=O)c2cnn(C)c2)c2[nH]cc(C#N)c12]

as described in Estrada, M. et al., WO2020210139.

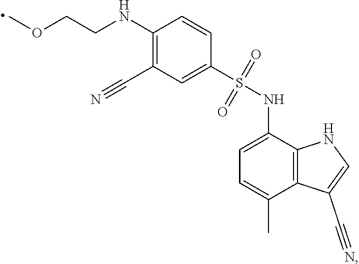 [SMILES: COCCNc1ccc(S(=O)(=O)Nc2ccc(C)c3c(C#N)c[nH]c23)cc1C#N]

as described in Gray, N. et al., WO2019147783.

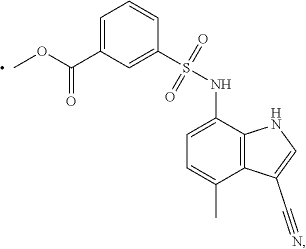 [SMILES: COC(=O)c1cccc(S(=O)(=O)Nc2ccc(C)c3c(C#N)c[nH]c23)c1]

as described in Gray, N. et al., WO2019147783.

In certain embodiments, the EPL is a radical of one of the above compounds, which is attached to L¹ through a modifiable oxygen, nitrogen, or carbon atom.

### D. Moiety for RBM23

In certain embodiments, the EPL is a moiety that binds to and/or degrades RBM23. Exemplary compounds that bind to and/or degrade RBM23 are reported in the literature, including:

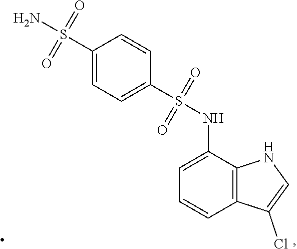 [SMILES: NS(=O)(=O)c1ccc(S(=O)(=O)Nc2cccc3c(Cl)c[nH]c23)cc1]

as described in Ting, T. et al., in Cell Reports (2019) 29: 1499.

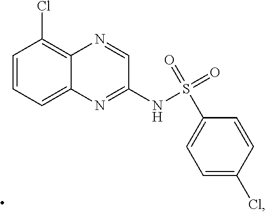 [SMILES: O=S(=O)(Nc1cnc2c(Cl)cccc2n1)c1ccc(Cl)cc1]

as described in Ting, T. et al., Cell Reports (2019) 29: 1499.

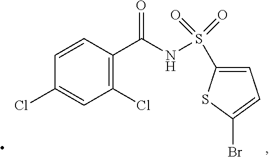 [SMILES: O=C(NS(=O)(=O)c1ccc(Br)s1)c1ccc(Cl)cc1Cl]

as described in Ting, T. et al., Cell Reports (2019) 29: 1499.

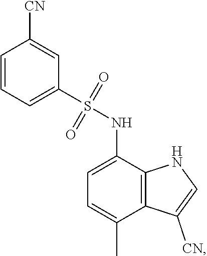 [SMILES: Cc1ccc(NS(=O)(=O)c2cccc(C#N)c2)c2[nH]cc(C#N)c12]

as described in Ting, T. et al., Cell Reports (2019) 29: 1499.

In certain embodiments, the EPL is a radical of one of the above compounds, which is attached to L¹ through a modifiable oxygen, nitrogen, or carbon atom.

### E. Moiety for IKZF1

In certain embodiments, the EPL is a moiety that binds to and/or degrades DNA-Binding Protein Ikaros (IKZF1). Exemplary compounds that bind to and/or degrade IKZF1 are reported in the literature, including:

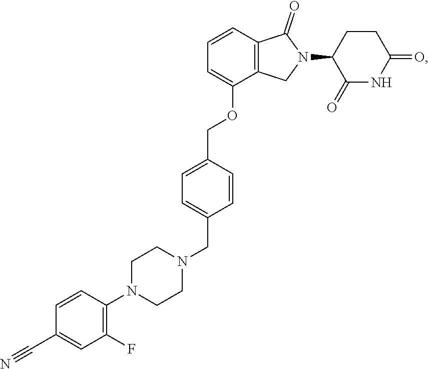 [SMILES: N#Cc1ccc(N2CCN(Cc3ccc(COc4cccc5c4CN([C@H]4CCC(=O)NC4=O)C5=O)cc3)CC2)c(F)c1]

as described in Alexander, M. D. et al., WO2019/014100.

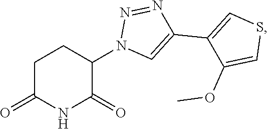 [SMILES: COc1cscc1-c1cn(C2CCC(=O)NC2=O)nn1]

as described in Watanabe, M. et al., WO2019/146773.

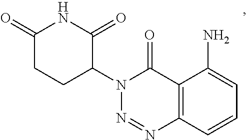 [SMILES: Nc1cccc2nnn(C3CCC(=O)NC3=O)c(=O)c12]

as described in Hwang, J. et al., WO2018/208123.

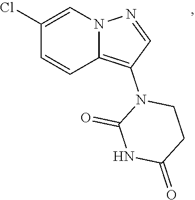 [SMILES: O=C1CCN(c2cnn3cc(Cl)ccc23)C(=O)N1]

as described in Axford, J. et al., WO2021/053555.

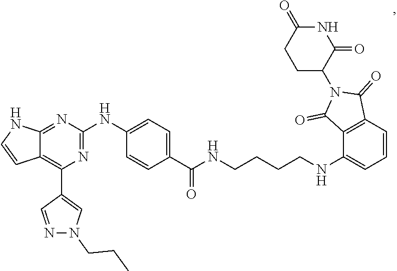 [SMILES: CCCn1cc(-c2nc(Nc3ccc(C(=O)NCCCCNc4cccc5c4C(=O)N(C4CCC(=O)NC4=O)C5=O)cc3)nc3[nH]ccc23)cn1]

as described in Min, J. et al., WO2021/022076.

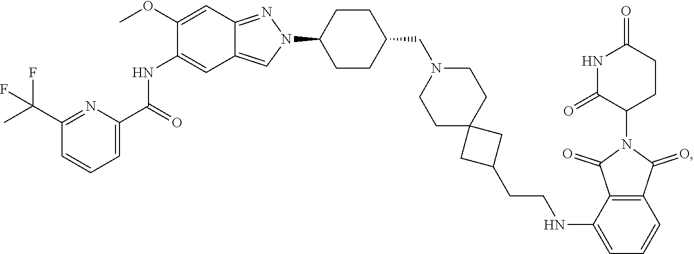 [SMILES: COc1cc2nn([C@H]3CC[C@H](CN4CCC5(CC4)CC(CCNc4cccc6c4C(=O)N(C4CCC(=O)NC4=O)C6=O)C5)CC3)cc2cc1NC(=O)c1cccc(C(C)(F)F)n1]

as described in Mainolfi, N. et al., WO2020/264499.

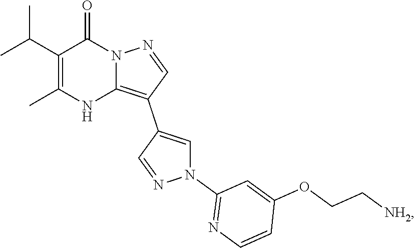 [SMILES: Cc1[nH]c2c(-c3cnn(-c4cc(OCCN)ccn4)c3)cnn2c(=O)c1C(C)C]

as described in Qi, J. et al., WO2020/263832.

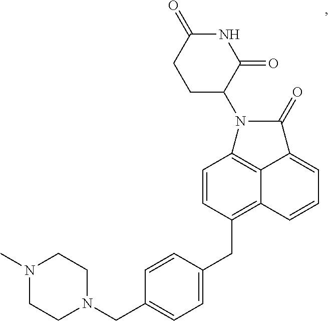 [SMILES: CN1CCN(Cc2ccc(Cc3ccc4c5c(cccc35)C(=O)N4C3CCC(=O)NC3=O)cc2)CC1]

as described in Henderson, J. et al., WO2020/210630.

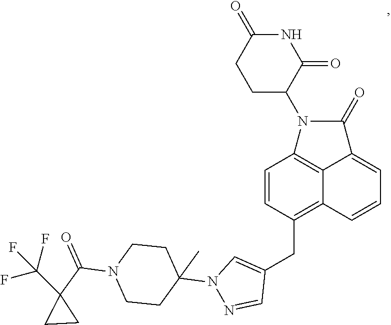 [SMILES: CC1(n2cc(Cc3ccc4c5c(cccc35)C(=O)N4C3CCC(=O)NC3=O)cn2)CCN(C(=O)C2(C(F)(F)F)CC2)CC1]

as described in Henderson, J. et al., WO2020/210630.

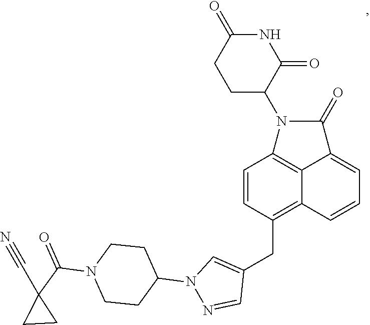 [SMILES: N#CC1(C(=O)N2CCC(n3cc(Cc4ccc5c6c(cccc46)C(=O)N5C4CCC(=O)NC4=O)cn3)CC2)CC1]

as described in Henderson, J. et al., WO2020/210630.

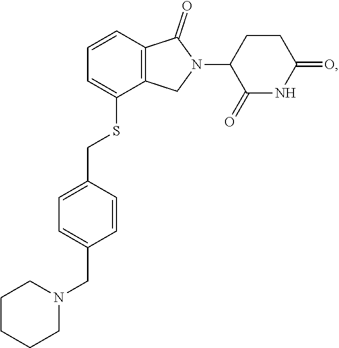 [SMILES: O=C1CCC(N2Cc3c(SCc4ccc(CN5CCCCC5)cc4)cccc3C2=O)C(=O)N1]

as described in Yang, X. et al., WO2020/173426.

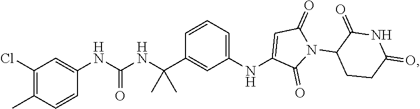 [SMILES: Cc1ccc(NC(=O)NC(C)(C)c2cccc(NC3=CC(=O)N(C4CCC(=O)NC4=O)C3=O)c2)cc1Cl]

as described in Verano, A. et al., WO2020/117759.

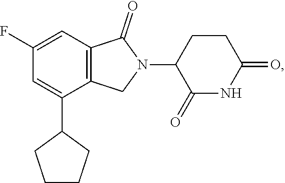 [SMILES: O=C1CCC(N2Cc3c(cc(F)cc3C3CCCC3)C2=O)C(=O)N1]

as described in Chan, K. et al., WO2020/102195.

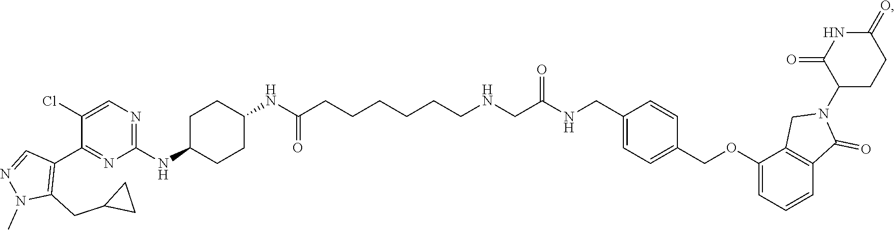 [SMILES: Cn1ncc(-c2nc(N[C@H]3CC[C@H](NC(=O)CCCCCCNCC(=O)NCc4ccc(COc5cccc6c5CN(C5CCC(=O)NC5=O)C6=O)cc4)CC3)ncc2Cl)c1CC1CC1]

as described in Chan, K. et al., WO2020/023782.

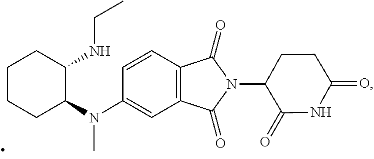 [SMILES: CCN[C@H]1CCCC[C@@H]1N(C)c1ccc2c(c1)C(=O)N(C1CCC(=O)NC1=O)C2=O]

as described in Beckwith, R. et al., WO2020/012337.

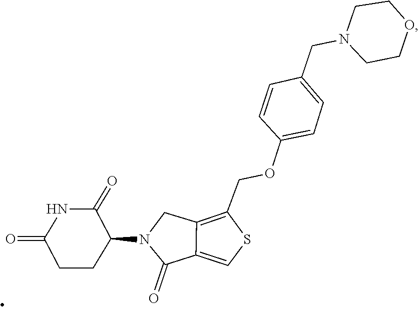 [SMILES: O=C1CC[C@H](N2Cc3c(csc3COc3ccc(CN4CCOCC4)cc3)C2=O)C(=O)N1]

as described in Chan, K. et al., WO2019/241271.

In certain embodiments, the EPL is a radical of one of the above compounds, which is attached to L¹ through a modifiable oxygen, nitrogen, or carbon atom.

### F. Moiety for IKZF3

In certain embodiments, the EPL is a moiety that binds to and/or degrades Zinc Finger Protein Aiolos (IKZF3). Exemplary compounds that bind to and/or degrade IKZF3 are reported in the literature, including:

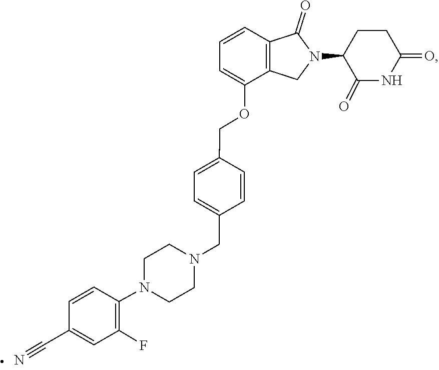 [SMILES: N#Cc1ccc(N2CCN(Cc3ccc(COc4cccc5c4CN([C@H]4CCC(=O)NC4=O)C5=O)cc3)CC2)c(F)c1]

as described in Alexander, M. D. et al., WO2019/014100.

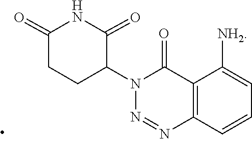 [SMILES: Nc1cccc2nnn(C3CCC(=O)NC3=O)c(=O)c12]

as described in Hwang, J. et al., WO2018/208123.

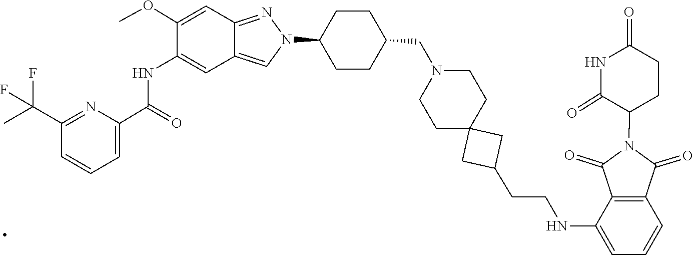 [SMILES: COc1cc2nn([C@H]3CC[C@H](CN4CCC5(CC4)CC(CCNc4cccc6c4C(=O)N(C4CCC(=O)NC4=O)C6=O)C5)CC3)cc2cc1NC(=O)c1cccc(C(C)(F)F)n1]

as described in Mainolfi, N. et al., WO2020/264499.

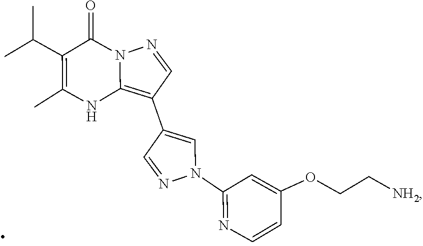 [SMILES: Cc1[nH]c2c(-c3cnn(-c4cc(OCCN)ccn4)c3)cnn2c(=O)c1C(C)C]

as described in Qi, J. et al., WO2020/263832.

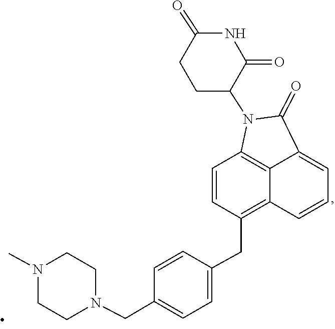 [SMILES: CN1CCN(Cc2ccc(Cc3ccc4c5c(cccc35)C(=O)N4C3CCC(=O)NC3=O)cc2)CC1]

as described in Henderson, J. et al., WO2020/210630.

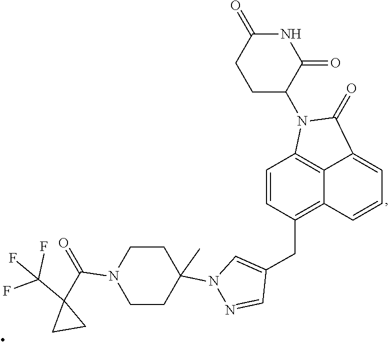 [SMILES: CC1(n2cc(Cc3ccc4c5c(cccc35)C(=O)N4C3CCC(=O)NC3=O)cn2)CCN(C(=O)C2(C(F)(F)F)CC2)CC1]

as described in Henderson, J. et al., WO2020/210630.

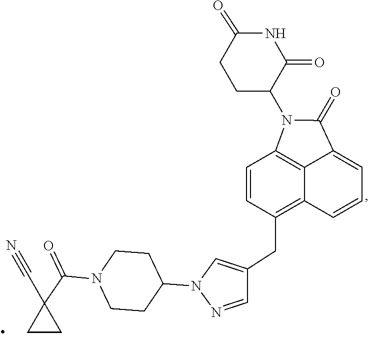 [SMILES: N#CC1(C(=O)N2CCC(n3cc(Cc4ccc5c6c(cccc46)C(=O)N5C4CCC(=O)NC4=O)cn3)CC2)CC1]

as described in Henderson, J. et al., WO2020/210630.

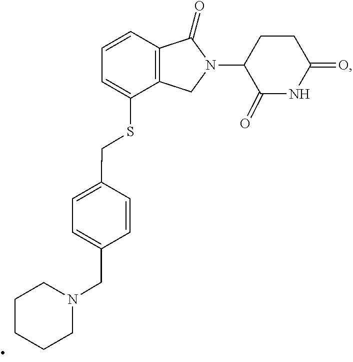 [SMILES: O=C1CCC(N2Cc3c(SCc4ccc(CN5CCCCC5)cc4)cccc3C2=O)C(=O)N1]

as described in Yang, X. et al., WO2020/173426.

In certain embodiments, the EPL is a radical of one of the above compounds, which is attached to L¹ through a modifiable oxygen, nitrogen, or carbon atom.

### G. Moiety for CDK4

In certain embodiments, the EPL is a moiety that binds to and/or degrades Cyclin-dependent kinase 4 (CDK4). Exemplary compounds that bind to and/or degrade CDK4 are reported in the literature, including:

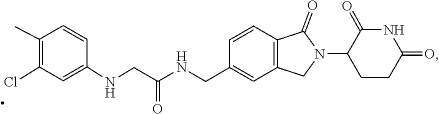 [SMILES: Cc1ccc(NCC(=O)NCc2ccc3c(c2)CN(C2CCC(=O)NC2=O)C3=O)cc1Cl]

as described in Zhao, M. et al., Biochem Biophys Res Comm (2021), 549 (21: 150.

In certain embodiments, the EPL is a radical of the above compound, which is attached to L¹ through a modifiable oxygen, nitrogen, or carbon atom.

### H. Moiety for PLK1

In certain embodiments, the EPL is a moiety that binds to and/or degrades Polo-like kinase 1 (PLK1). Exemplary compounds that bind to and/or degrade PLK1 are reported in the literature, including:

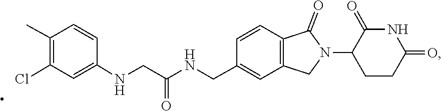 [SMILES: Cc1ccc(NCC(=O)NCc2ccc3c(c2)CN(C2CCC(=O)NC2=O)C3=O)cc1Cl]

as described in Li, L. et al., Mol Ther Onc (2020), 18: 215.

In certain embodiments, the EPL is a radical of the above compound, which is attached to L¹ through a modifiable oxygen, nitrogen, or carbon atom.

As defined generally above, the TPL is a moiety that binds to a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4. In certain embodiments, the TPL is a moiety that binds to a target protein selected from KRAS, HER2, BTK, EGFR, androgen receptor protein, estrogen receptor protein, or ALK. In certain embodiments, TPL is a moiety that binds to a target protein selected from IDH1, FLT3, FGFR1, FGFR4, FGFR2, FGFR3, ERK1, ERK2, FGR, HER3, or HER4. In certain embodiments, TPL is a moiety that binds to a target protein selected from KRAS. In certain embodiments, TPL is a moiety that binds to a target protein selected from HER2. In certain embodiments, TPL is a moiety that binds to a target protein selected from BTK. In certain embodiments, TPL is a moiety that binds to a target protein selected from EGFR. In certain embodiments, TPL is a moiety that binds to a target protein selected from androgen receptor protein. In certain embodiments, TPL is a moiety that binds to a target protein selected from estrogen receptor protein. In certain embodiments, TPL is a moiety that binds to a target protein selected from ALK. In certain embodiments, TPL is a moiety that binds to a target protein selected from IDH1. In certain embodiments, TPL is a moiety that binds to a target protein selected from FLT3. In certain embodiments, TPL is a moiety that binds to a target protein selected from FGFR1. In certain embodiments, TPL is a moiety that binds to a target protein selected from FGFR4. In certain embodiments, TPL is a moiety that binds to a target protein selected from FGFR2. In certain embodiments, TPL is a moiety that binds to a target protein selected from FGFR3. In certain embodiments, TPL is a moiety that binds to a target protein selected from ERK1. In certain embodiments, TPL is a moiety that binds to a target protein selected from ERK2. In certain embodiments, TPL is a moiety that binds to a target protein selected from FGR. In certain embodiments, TPL is a moiety that binds to a target protein selected from HER3. In certain embodiments, TPL is a moiety that binds to a target protein selected from HER4. In certain embodiments, TPL is selected from those depicted in the compounds in any one of Tables 1, 2, 3, or 4 below.

As defined generally above, L¹ is a bond, **-linker-O—, or **-linker-N(R⁵)—, where ** is a point of attachment to EPL, and L¹ is connected to a nitrogen atom of EPL when L¹ is a bond. In certain embodiments, L¹ is a bond. In certain embodiments, L¹ is **-linker-O—. In certain embodiments, L¹ is **-linker-N(R⁵)—, where ** is a point of attachment to EPL. In certain embodiments, L¹ is one of the following: —O—, —N(H)—, —N(C₁₋₄ alkyl)-, —OC(O)—**, —N(H)C(O)—**, —N(C₁₋₄ alkyl)C(O)—**, —O—(C₁₋₆ alkylene)-**, —N(H)—(C₁₋₆ alkylene)-**, —N(C₁₋₄ alkyl)-(C₁₋₆ alkylene)-**, —OC(O)—(C₁₋₆ alkylene)-**, —N(H)C(O)—(C₁₋₆ alkylene)-**, —N(C₁₋₄ alkyl)C(O)—(C₁₋₆ alkylene)-**, —OC(O)N(H)—(C₁₋₆ alkylene)-**, —OC(O)N(C₁₋₄ alkyl)-(C₁₋₆ alkylene)-**, —N(H)C(O)₂—(C₁₋₆ alkylene)-**, —N(C₁₋₄ alkyl)C(O)₂—(C₁₋₆ alkylene)-**, or

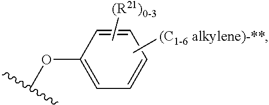

wherein R²¹ represents independently for each occurrence halo, C₁₋₄ alkyl, C₁₋₄haloalkyl, hydroxyl, C₁₋₄ alkoxyl, or C₃₋₆ cycloalkyl, where ** is a point of attachment to EPL. In certain embodiments, L¹ is —O—. In certain embodiments, L¹ is —OC(O)—**. In certain embodiments, L¹ is selected from those depicted in the compounds in any one of Tables 1, 2, 3, or 4 below.

As defined generally above, L² is a bond or a linker. In certain embodiments, L² is a bond. In certain embodiments, L² is a linker. In certain embodiments, L² is selected from those depicted in the compounds in any one of Tables 1, 2, 3, or 4 below.

In certain embodiments, the linker is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, the linker has the formula —(C₀₋₁₂ alkylene)-(optionally substituted 3-40 membered heteroalkylene)-(C₀₋₁₂ alkylene)-. In certain embodiments, the linker is C₄₋₁₄ alkylene.

### Part B: Compounds of Formula I-A

Another aspect of the invention provides a compound represented by Formula I-A:

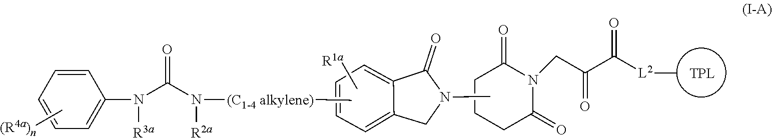

or a pharmaceutically acceptable salt thereof; wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

TPL is a moiety that binds to a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4; and
L² is a bond or a linker.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

As defined generally above, R^{1a} is hydrogen, halo, or C₁₋₄ alkyl. In certain embodiments, R^{1a} is hydrogen. In certain embodiments, R^{1a} is halo. In certain embodiments, R^{1a} is C₁₋₄ alkyl. In certain embodiments, R^{1a} is selected from those depicted in the compounds in any one of Table 1 below. In certain embodiments, R^{1a} is selected from those depicted in the compounds in any one of Table 1-A below.

As defined generally above, R^{2a} and R^{3a} each represent independently for each occurrence hydrogen or C₁₋₄ alkyl. In certain embodiments, R^{2a} is hydrogen. In certain embodiments, R^{3a} is hydrogen. In certain embodiments, R^{2a} is C₁₋₄ alkyl. In certain embodiments, R^{3a} is C₁₋₄ alkyl. In certain embodiments, R^{2a} is selected from those depicted in the compounds in any one of Table 1 below. In certain embodiments, R^{3a} is selected from those depicted in the compounds in any one of Table 1 below. In certain embodiments, R^{3a} is selected from those depicted in the compounds in any one of Table 1-A below.

In certain embodiments, R^{1a}, R^{2a}, and R^{3a} are hydrogen.

As defined generally above, R^{4a} represents independently for each occurrence halo, C₁₋₄ alkyl, C₁₋₄ haloalkyl, hydroxyl, or C₁₋₄ alkoxyl. In certain embodiments, R^{4A} represents independently for each occurrence halo or C₁₋₄ alkyl. In certain embodiments, R^{4A} represents independently for each occurrence halo. In certain embodiments, R^{4A} represents independently for each occurrence C₁₋₄ alkyl. In certain embodiments, R^{4A} represents independently for each occurrence C₁₋₄ haloalkyl. In certain embodiments, R^{4A} represents independently for each occurrence hydroxyl or C₁₋₄ alkoxyl.

In certain embodiments, R^{4a} represents independently for each occurrence halo or C₁₋₄ alkyl, and n is 2. In certain embodiments, R^{4a} is selected from those depicted in the compounds in any one of Table 1 below. In certain embodiments, R^{4a} is selected from those depicted in the compounds in any one of Table 1-A below.

As defined generally above, n is 0, 1, 2, or 3. In certain embodiments, n is 0. In certain embodiments, n is 1. In certain embodiments, n is 2. In certain embodiments, n is selected from those depicted in the compounds in any one of Table 1 below. In certain embodiments, n is selected from those depicted in the compounds in any one of Table 1-A below.

As defined generally above, TPL is a moiety that binds to a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4. In certain embodiments, the TPL is a moiety that binds to a target protein selected from KRAS, HER2, BTK, EGFR, androgen receptor protein, estrogen receptor protein, or ALK. In certain embodiments, TPL is a moiety that binds to a target protein selected from IDH1, FLT3, FGFR1, FGFR4, FGFR2, FGFR3, ERK1, ERK2, FGR, HER3, or HER4. In certain embodiments, TPL is a moiety that binds to a target protein selected from KRAS. In certain embodiments, TPL is a moiety that binds to a target protein selected from HER2. In certain embodiments, TPL is a moiety that binds to a target protein selected from BTK. In certain embodiments, TPL is a moiety that binds to a target protein selected from EGFR. In certain embodiments, TPL is a moiety that binds to a target protein selected from androgen receptor protein. In certain embodiments, TPL is a moiety that binds to a target protein selected from estrogen receptor protein. In certain embodiments, TPL is a moiety that binds to a target protein selected from ALK. In certain embodiments, TPL is a moiety that binds to a target protein selected from IDH1. In certain embodiments, TPL is a moiety that binds to a target protein selected from FLT3. In certain embodiments, TPL is a moiety that binds to a target protein selected from FGFR1. In certain embodiments, TPL is a moiety that binds to a target protein selected from FGFR4. In certain embodiments, TPL is a moiety that binds to a target protein selected from FGFR2. In certain embodiments, TPL is a moiety that binds to a target protein selected from FGFR3. In certain embodiments, TPL is a moiety that binds to a target protein selected from ERK1. In certain embodiments, TPL is a moiety that binds to a target protein selected from ERK2. In certain embodiments, TPL is a moiety that binds to a target protein selected from FGR. In certain embodiments, TPL is a moiety that binds to a target protein selected from HER3. In certain embodiments, TPL is a moiety that binds to a target protein selected from HER4. In certain embodiments, TPL is selected from those depicted in the compounds in any one of Table 1 below.

As defined generally above, L² is a bond or a linker. In certain embodiments, L² is a bond. In certain embodiments, L² is a linker. In certain embodiments, L² is selected from those depicted in the compounds in any one of Table 1 below.

In certain embodiments, the linker is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, the linker has the formula —(C₀₋₁₂ alkylene)-(optionally substituted 3-40 membered heteroalkylene)-(C₀₋₁₂ alkylene)-. In certain embodiments, the linker is C₄₋₁₄ alkylene.

### Part C: Exemplary Further Description of TPL Component of Compounds of Formula I and I-A

Exemplarily moieties for the TPL component are described in more detail below.

### Moiety for KRAS

In certain embodiments, the TPL is a moiety that binds to KRas. Exemplary compounds that bind to KRas are reported in the literature, such as MRTX849 and AMG510. A radical of such compounds reported in the literature that bind KRas are amenable for use in the present invention.

In certain embodiments, the TPL is one of the following:

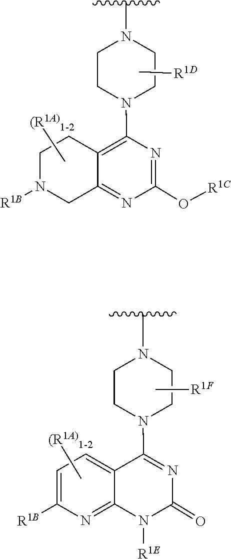

wherein:
R^{1A} represents independently for each occurrence hydrogen, halo, hydroxyl, C₁₋₄ alkyl, or C₁₋₄ alkoxyl; R^{1B} is C₆₋₁₂ aryl or 6-12 membered heteroaryl containing 1, 2 or 3 heteroatoms selected from nitrogen, oxygen, or sulfur, wherein the aryl and heteroaryl are optionally substituted by 1, 2, or 3 substituents independently selected from halo, hydroxyl, C₁₋₄ alkyl, or C₁₋₄ alkoxyl;
R^{1C} is —(C₁₋₆ alkylene)-3-7 membered saturated mono-cyclic or bicylic heterocyclyl containing 1, 2, or 3 heteroatoms selected from nitrogen, oxygen, and sulfur;
R^{1D} is —(C₁₋₆ alkylene)-CN;
R^{1E} is C₆₋₁₂ aryl or 6-12 membered heteroaryl containing 1, 2 or 3 heteroatoms selected from nitrogen, oxygen, or sulfur, wherein the aryl and heteroaryl are optionally substituted by 1, 2, or 3 substituents independently selected from halo, hydroxyl, C₁₋₄ alkyl, or C₁₋₄ alkoxyl; and
R^{1F} is C₁₋₆ alkyl.

In certain embodiments, the TPL is the following:

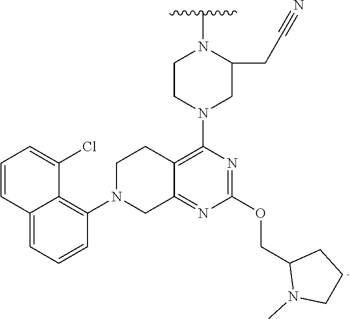 [SMILES: *N1CCN(c2nc(OCC3CCCN3C)nc3c2CCN(c2cccc4cccc(Cl)c24)C3)CC1CC#N]

In certain embodiments, the TPL is the following:

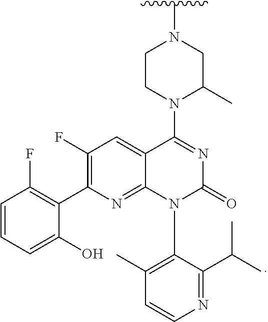 [SMILES: *N1CCN(c2nc(=O)n(-c3c(C)ccnc3C(C)C)c3nc(-c4c(O)cccc4F)c(F)cc23)C(C)C1]

In certain embodiments, TPL is one of the following:

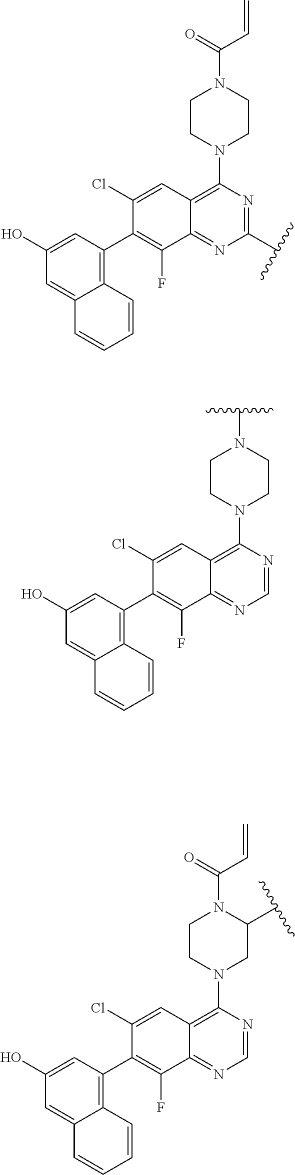 [SMILES: *c1nc(N2CCN(C(=O)C=C)CC2)c2cc(Cl)c(-c3cc(O)cc4ccccc34)c(F)c2n1 ; *N1CCN(c2ncnc3c(F)c(-c4cc(O)cc5ccccc45)c(Cl)cc23)CC1 ; *C1CN(c2ncnc3c(F)c(-c4cc(O)cc5ccccc45)c(Cl)cc23)CCN1C(=O)C=C]

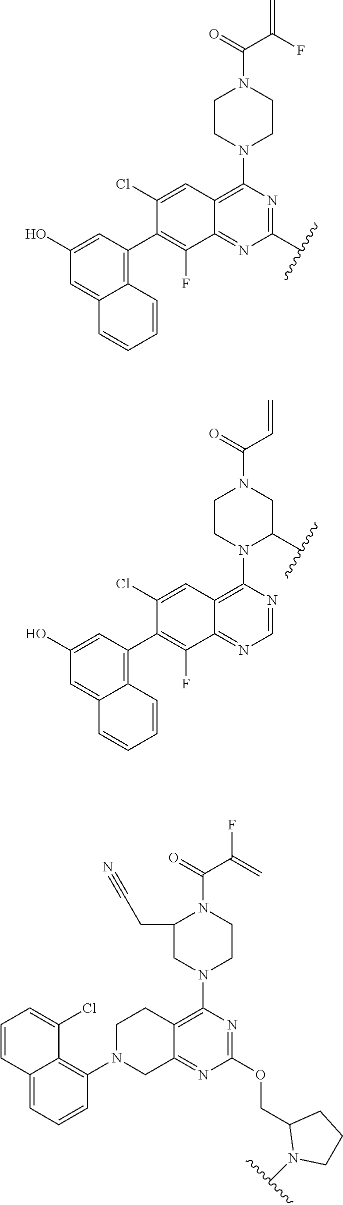 [SMILES: *c1nc(N2CCN(C(=O)C(=C)F)CC2)c2cc(Cl)c(-c3cc(O)cc4ccccc34)c(F)c2n1 ; *C1CN(C(=O)C=C)CCN1c1ncnc2c(F)c(-c3cc(O)cc4ccccc34)c(Cl)cc12 ; *N1CCCC1COc1nc2c(c(N3CCN(C(=O)C(=C)F)C(CC#N)C3)n1)CCN(c1cccc3cccc(Cl)c13)C2]

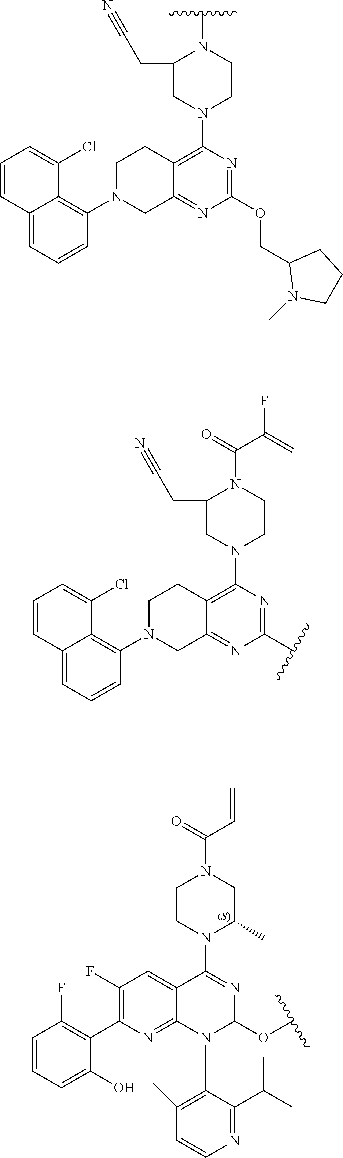

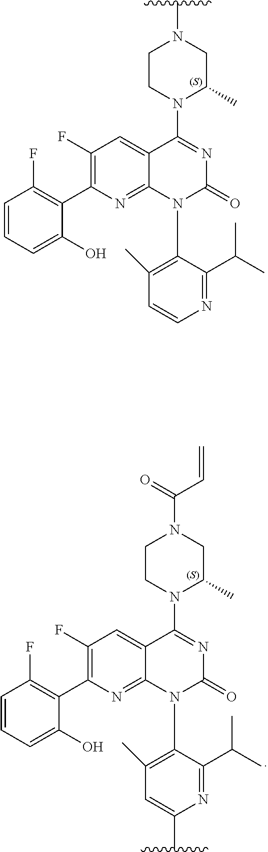

In certain embodiments, the TPL is one of the following:

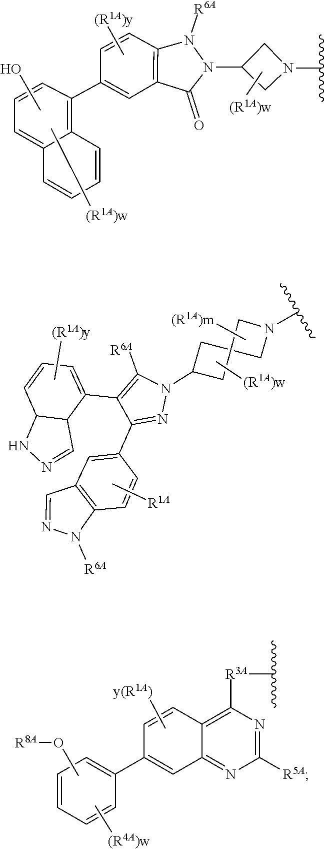

wherein:
R^{1A} and R^{4A} each represent independently for each occurrence hydrogen, halo, hydroxyl, C₁₋₄ alkyl, C₁₋₄ alkoxyl, or C₃₋₆ cycloalkyl;
R^{2A} represents independently for each occurrence hydrogen or C₁₋₄ alkyl;
R^{3A} is a 3-7 membered saturated heterocyclylene containing 1, 2, or 3 heteroatoms independently selected from oxygen, nitrogen, and sulfur, wherein the heterocyclylene is substituted with 0, 1, 2, or 3 substituents independently selected from halo and C₁₋₄ alkyl;
R^{5A} is hydrogen, halo, hydroxyl, or C₁₋₄ alkyl;
R^{6A} is C₁₋₆ alkyl or C₃₋₆ cycloalkyl;
R^{7A} is C₁₋₆ alkylene)—N(R^{8A})₂;
R^{8A} is hydrogen, C₁₋₆ alkyl, or C₃₋₆ cycloalkyl;
y and w each represent independently for each occurrence 1 or 2.

In certain embodiments, the TPL is

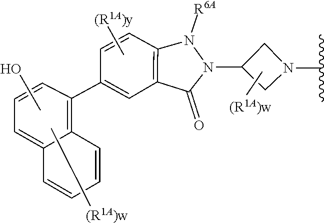

where the variables are as defined above.

In certain embodiments, the TPL is

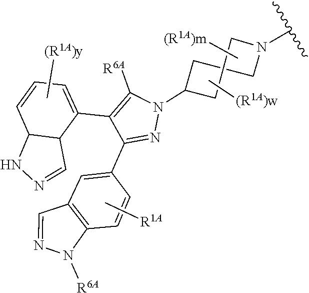

where the variables are as defined above. In certain embodiments, the TPL is

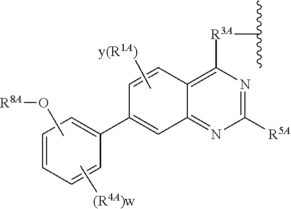

where the variables are as defined above.

In certain embodiments, R^{1A} is hydrogen. In certain embodiments, R^{1A} is halo. In certain embodiments, R^{1A} is hydroxyl. In certain embodiments, R^{1A} is C₁₋₄ alkyl. In certain embodiments, R^{1A} is C₁₋₄ alkoxyl. In certain embodiments, R^{1A} is C₃₋₆ cycloalkyl.

In certain embodiments, R^{4A} is hydrogen. In certain embodiments, R^{4A} is halo. In certain embodiments, R^{4A} is hydroxyl. In certain embodiments, R^{4A} is C₁₋₄ alkyl. In certain embodiments, R^{4A} is C₁₋₄ alkoxyl. In certain embodiments, R^{4A} is C₃₋₆ cycloalkyl.

In certain embodiments, R^{2A} is hydrogen. In certain embodiments, R^{2A} is C₁₋₄ alkyl.

In certain embodiments, R^{3A} is a 5-6 membered saturated heterocyclylene containing 1 or 2 heteroatoms independently selected from oxygen, nitrogen, and sulfur, wherein the heterocyclylene is substituted with 0, 1, 2, or 3 substituents independently selected from halo and C₁₋₄ alkyl.

In certain embodiments, R^{5A} is hydrogen. In certain embodiments, R^{5A} is halo. In certain embodiments, R^{5A} is hydroxyl. In certain embodiments, R^{5A} is C₁₋₄ alkyl.

In certain embodiments, R^{6A} is C₁₋₆ alkyl. In certain embodiments, R^{6A} is C₃₋₆ cycloalkyl. In certain embodiments, R^{7A} is C₁₋₃ alkylene)-N(R^{8A})₂.

In certain embodiments, R^{8A} is hydrogen.

In certain embodiments, R^{8A} is C₁₋₆ alkyl. In certain embodiments, R^{8A} is C₃₋₆ cycloalkyl.

In certain embodiments, y is 1. In certain embodiments, y is 2. In certain embodiments, w is 1. In certain embodiments, w is 2.

In certain embodiments, the TPL is one of the following:

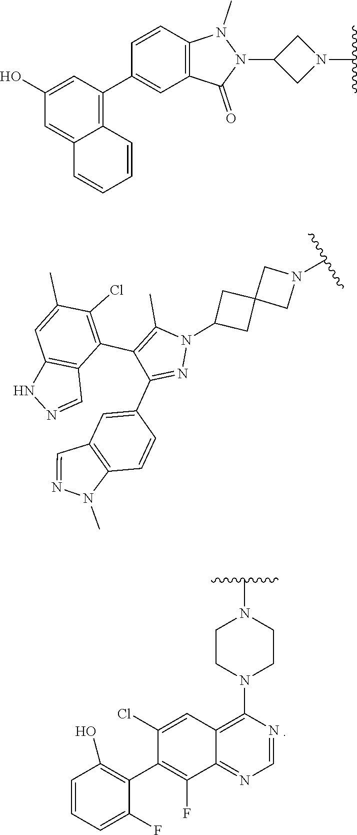

In certain embodiments, the TPL is one of the following:

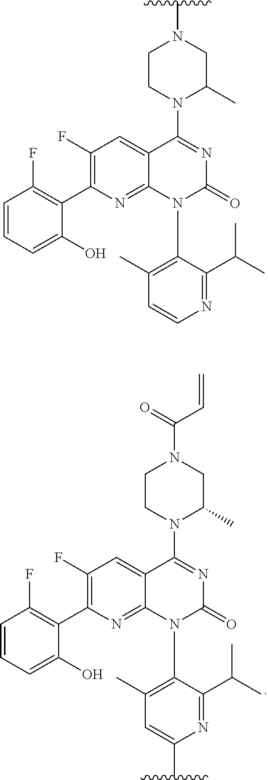

In certain embodiments, the TPL is one of the following:

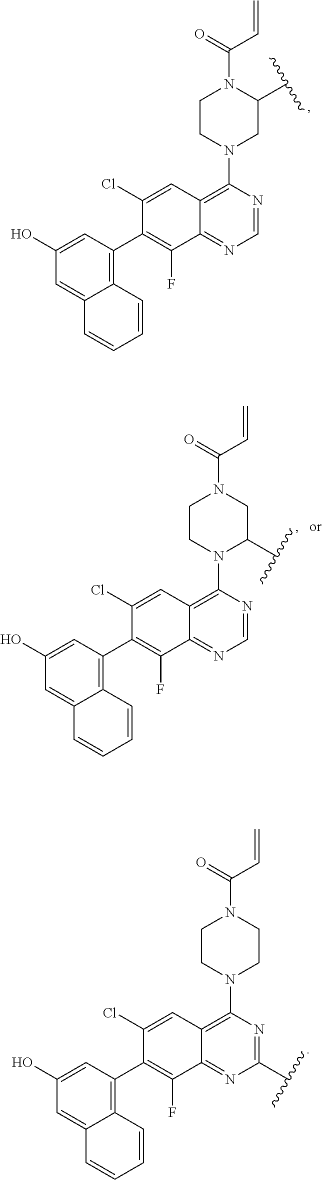 [SMILES: *C1CN(c2ncnc3c(F)c(-c4cc(O)cc5ccccc45)c(Cl)cc23)CCN1C(=O)C=C ; *C1CN(C(=O)C=C)CCN1c1ncnc2c(F)c(-c3cc(O)cc4ccccc34)c(Cl)cc12 ; *c1nc(N2CCN(C(=O)C=C)CC2)c2cc(Cl)c(-c3cc(O)cc4ccccc34)c(F)c2n1]

In certain embodiments, the TPL is one of the following:

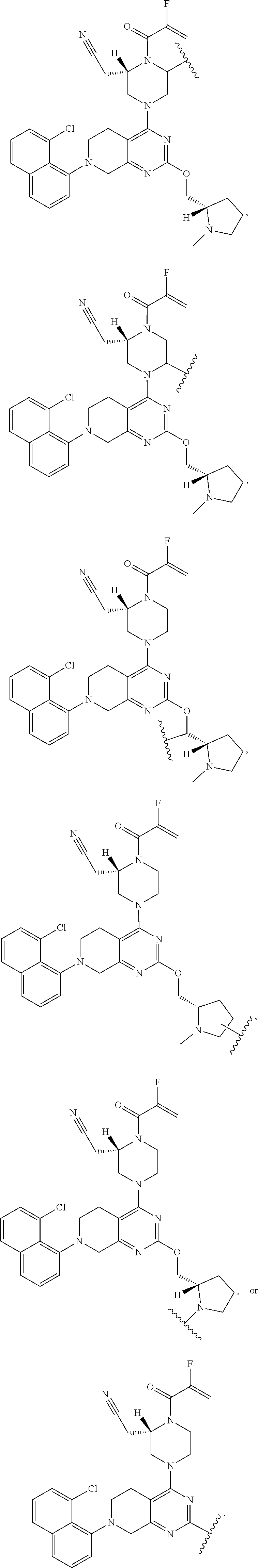

In certain embodiments, the TPL is

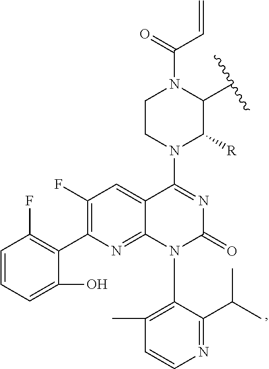

wherein R═H, Me, Et, CH₂OH, CH₂NH₂, CH₂NHR′, OH, or NH₂; and R′ is alkyl, alkenyl, amido, amino, aminoalky, or alkoxy. In certain embodiments, the TPL is

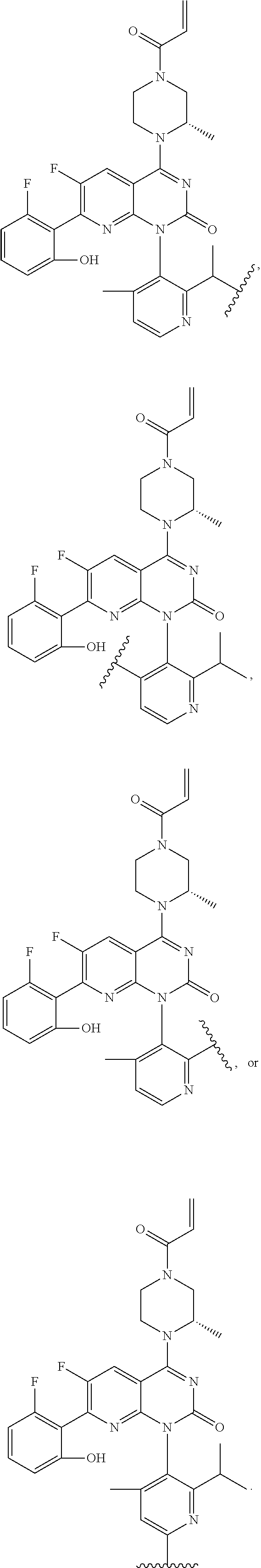 [SMILES: *C(C)c1nccc(C)c1-n1c(=O)nc(N2CCN(C(=O)C=C)C[C@@H]2C)c2cc(F)c(-c3c(O)cccc3F)nc21 ; *c1ccnc(C(C)C)c1-n1c(=O)nc(N2CCN(C(=O)C=C)C[C@@H]2C)c2cc(F)c(-c3c(O)cccc3F)nc21 ; *c1nccc(C)c1-n1c(=O)nc(N2CCN(C(=O)C=C)C[C@@H]2C)c2cc(F)c(-c3c(O)cccc3F)nc21 ; *c1cc(C)c(-n2c(=O)nc(N3CCN(C(=O)C=C)C[C@@H]3C)c3cc(F)c(-c4c(O)cccc4F)nc32)c(C(C)C)n1]

In certain embodiments, the TPL is

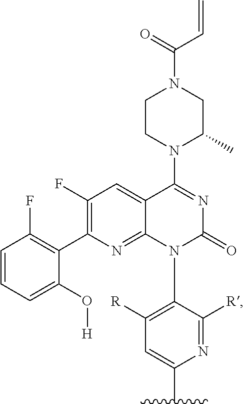

wherein R is H, a linker (e.g., alkyl); and R′ is H or a linker (e.g., alkyl). In certain embodiments, the TPL is

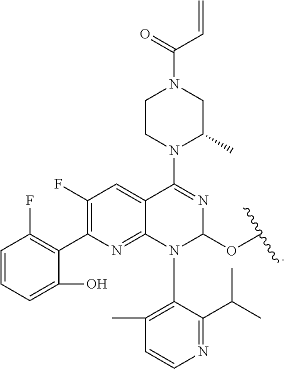 [SMILES: *OC1N=C(N2CCN(C(=O)C=C)C[C@@H]2C)c2cc(F)c(-c3c(O)cccc3F)nc2N1c1c(C)ccnc1C(C)C]

In certain embodiments, the TPL is

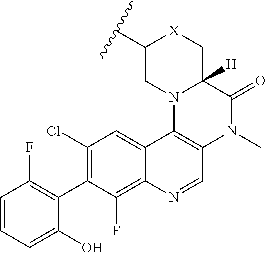

wherein X is NH, NR^{a}, CH₂, CHR^{a}, or C(R^{a})₂; and R^{a} is alkyl, alkenyl, amido, amino, aminoalky, or alkoxy.

In certain embodiments, the TPL is

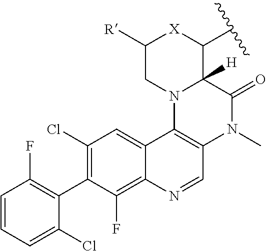

wherein X is NH, NR^{a}, CH₂, CHR^{a}, or C(R^{a})₂; R^{a} is alkyl, alkenyl, amido, amino, aminoalky, or alkoxy; and R′ is H, Me, or Et. In certain embodiments, the TPL is

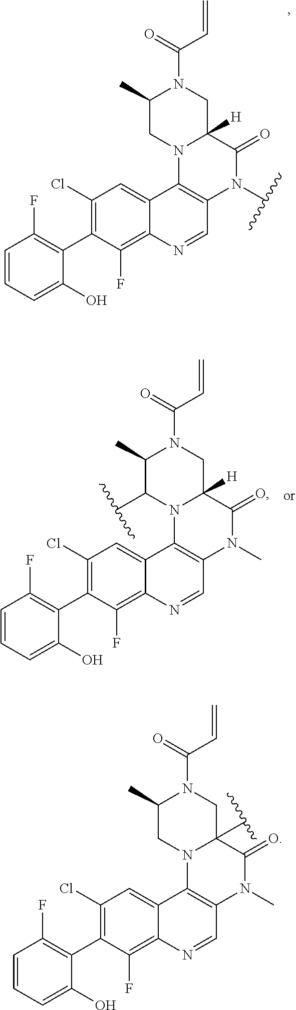 [SMILES: *N1C(=O)[C@H]2CN(C(=O)C=C)[C@H](C)CN2c2c1cnc1c(F)c(-c3c(O)cccc3F)c(Cl)cc21 ; *C1[C@@H](C)N(C(=O)C=C)C[C@@H]2C(=O)N(C)c3cnc4c(F)c(-c5c(O)cccc5F)c(Cl)cc4c3N12 ; *C12CN(C(=O)C=C)[C@H](C)CN1c1c(cnc3c(F)c(-c4c(O)cccc4F)c(Cl)cc13)N(C)C2=O]

In certain embodiments, the TPL is a moiety that binds to a mutated Kirsten rat sarcoma 2 viral oncogene homolog. Compounds that bind mutated Kirsten rat sarcoma 2 viral oncogene homolog are reported in the literature, which include:

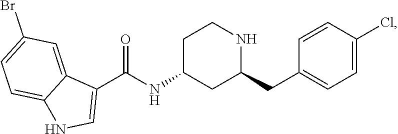 [SMILES: O=C(N[C@@H]1CCN[C@@H](Cc2ccc(Cl)cc2)C1)c1c[nH]c2ccc(Br)cc12]

as described in Jansen, J. M. et al., 24th Int Symp Med Chem (August 28-September 1, Manchester) 2016, Abstract LE007;

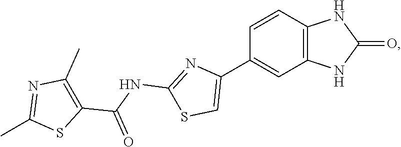 [SMILES: Cc1nc(C)c(C(=O)Nc2nc(-c3ccc4[nH]c(=O)[nH]c4c3)cs2)s1]

as described in Rabizadeh, S. et al., WO 2016/161361;

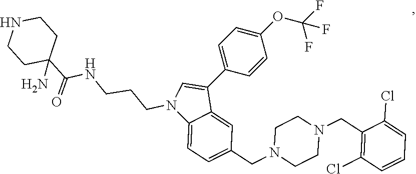 [SMILES: NC1(C(=O)NCCCn2cc(-c3ccc(OC(F)(F)F)cc3)c3cc(CN4CCN(Cc5c(Cl)cccc5Cl)CC4)ccc32)CCNCC1]

as described in Welsch, M. E. et al., Cell 2017, vol. 168(5), page 878;

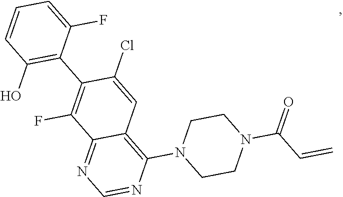 [SMILES: C=CC(=O)N1CCN(c2ncnc3c(F)c(-c4c(O)cccc4F)c(Cl)cc23)CC1]

as described in Wijeratne, A. et al., ACS Med Chem Lett 2018, vol. 9(6), page 557.

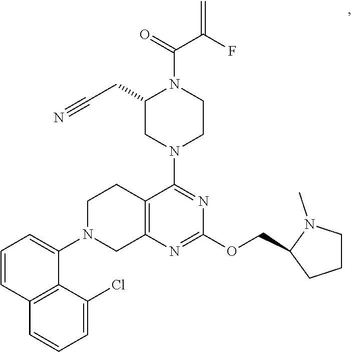 [SMILES: C=C(F)C(=O)N1CCN(c2nc(OC[C@@H]3CCCN3C)nc3c2CCN(c2cccc4cccc(Cl)c24)C3)C[C@@H]1CC#N]

as described in Blake, J. et al., WO 2019/099524

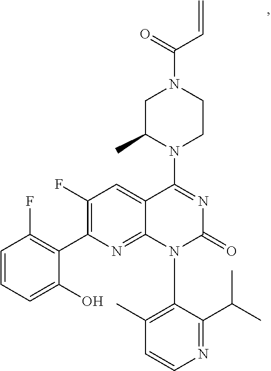 [SMILES: C=CC(=O)N1CCN(c2nc(=O)n(-c3c(C)ccnc3C(C)C)c3nc(-c4c(O)cccc4F)c(F)cc23)[C@@H](C)C1]

as described in Lanman, B. et al., WO 2018/217651

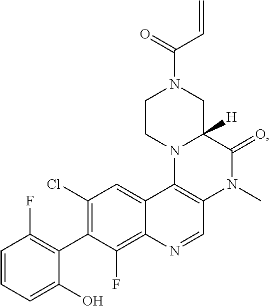 [SMILES: C=CC(=O)N1CCN2c3c(cnc4c(F)c(-c5c(O)cccc5F)c(Cl)cc34)N(C)C(=O)[C@H]2C1]

as described in Kettle, J. et al., WO 2019/110751.

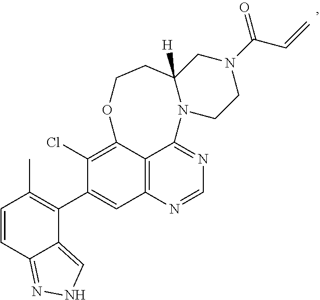 [SMILES: C=CC(=O)N1CCN2c3ncnc4cc(-c5c(C)ccc6n[nH]cc56)c(Cl)c(c34)OCC[C@@H]2C1]

as described in Kettle, J. et al., WO 2018/206539.

In certain embodiments, the TPL is a radical of one of the above compounds, which is attached to L² through a modifiable oxygen, nitrogen, or carbon atom.

### Moiety for HER2

In certain embodiments, the TPL is a moiety that binds human epidermal growth factor receptor 2 (HER2). Compounds that inhibit and/or bind to HER2 are reported in the literature, which include:

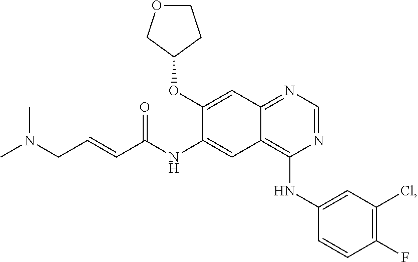 [SMILES: CN(C)C/C=C/C(=O)Nc1cc2c(Nc3ccc(F)c(Cl)c3)ncnc2cc1O[C@H]1CCOC1]

as described in Chen, J. et al., WO 2015/023703;

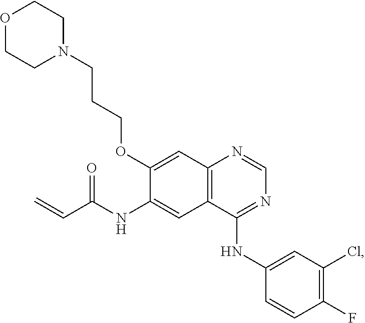 [SMILES: C=CC(=O)Nc1cc2c(Nc3ccc(F)c(Cl)c3)ncnc2cc1OCCCN1CCOCC1]

as described in Huang, Z. et al., WO 2012/027960;

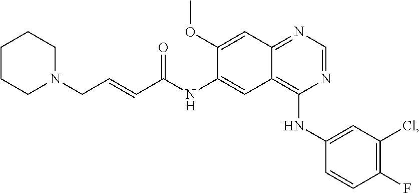 [SMILES: COc1cc2ncnc(Nc3ccc(F)c(Cl)c3)c2cc1NC(=O)/C=C/CN1CCCCC1]

as described in Wu, F. et al., WO 2012/159457;

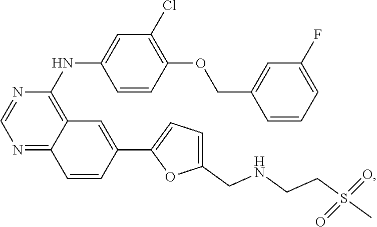 [SMILES: CS(=O)(=O)CCNCc1ccc(-c2ccc3ncnc(Nc4ccc(OCc5cccc(F)c5)c(Cl)c4)c3c2)o1]

as described in Wu, F. et al., WO 2012/159457;

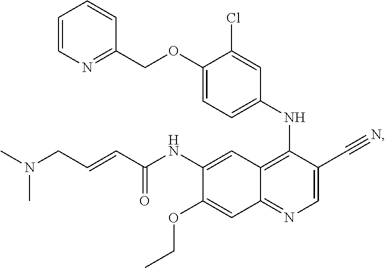 [SMILES: CCOc1cc2ncc(C#N)c(Nc3ccc(OCc4ccccn4)c(Cl)c3)c2cc1NC(=O)/C=C/CN(C)C]

as described in Wissner, A. et al., WO 2005/034955;

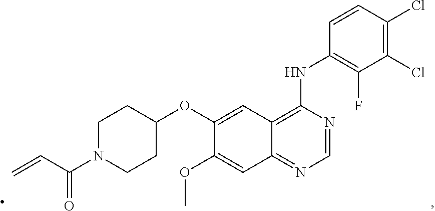 [SMILES: C=CC(=O)N1CCC(Oc2cc3c(Nc4ccc(Cl)c(Cl)c4F)ncnc3cc2OC)CC1]

as described in Li, Z. et al., WO 2019/149164;

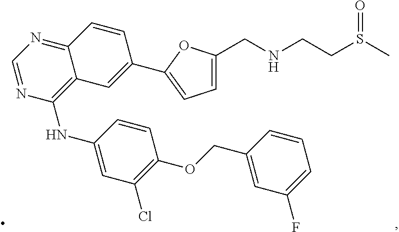 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

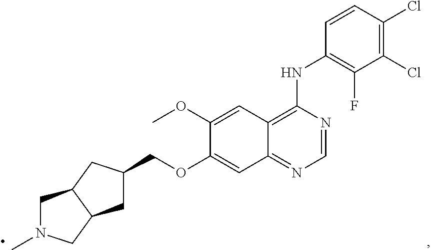 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

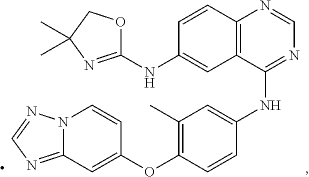 [SMILES: Cc1cc(Nc2ncnc3ccc(NC4=NC(C)(C)CO4)cc23)ccc1Oc1ccn2ncnc2c1]

as described in Xia, G. et al., WO 2017/148391;

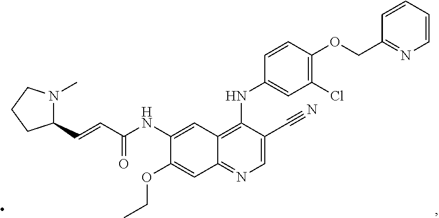 [SMILES: CCOc1cc2ncc(C#N)c(Nc3ccc(OCc4ccccn4)c(Cl)c3)c2cc1NC(=O)/C=C/[C@H]1CCCN1C]

as described in Li, X. et al., WO 2012/122865.

In certain embodiments, the TPL is a radical of one of the above compounds, which is attached to L² through a modifiable oxygen, nitrogen, or carbon atom.

In certain embodiments, the TPL is one of the following:

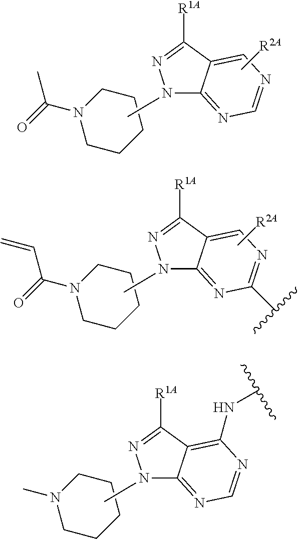

wherein:
R^{1A} is —C(O)(NR^{5A})-(phenyl optionally substituted with 1, 2, 3, or 5 substituents independently selected from halo, hydroxyl, C₁₋₄ alkyl, C₁₋₄ alkoxyl, and —(C₁₋₄ alkylene-C(O)N(R^{5A})(R^{6A}));
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

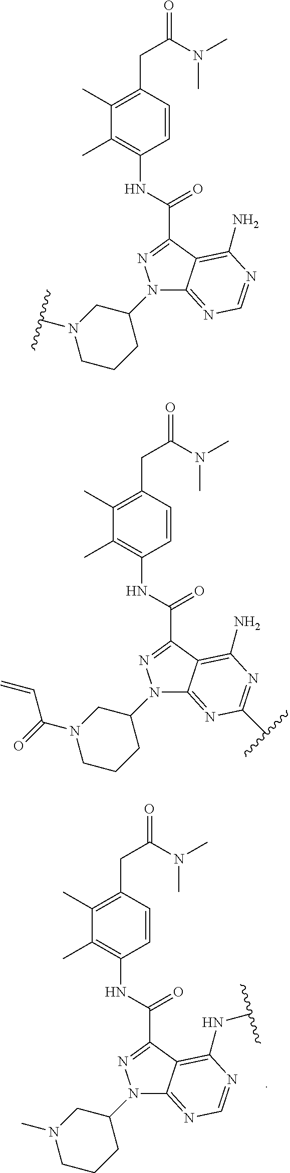 [SMILES: *N1CCCC(n2nc(C(=O)Nc3ccc(CC(=O)N(C)C)c(C)c3C)c3c(N)ncnc32)C1 ; *c1nc(N)c2c(C(=O)Nc3ccc(CC(=O)N(C)C)c(C)c3C)nn(C3CCCN(C(=O)C=C)C3)c2n1 ; *Nc1ncnc2c1c(C(=O)Nc1ccc(CC(=O)N(C)C)c(C)c1C)nn2C1CCCN(C)C1]

In certain embodiments, the TPL is one of the following:

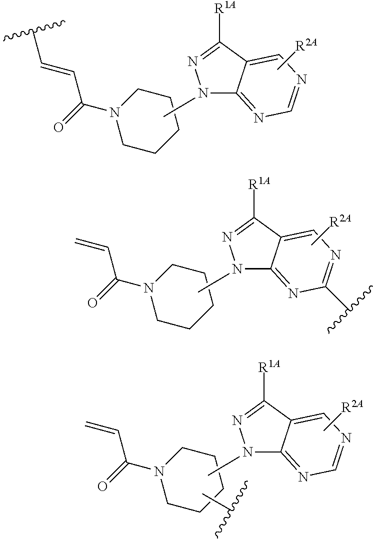

wherein:
R^{1A} is —C(O)(NR^{5A})-(phenyl optionally substituted with 1, 2, 3, or 5 substituents independently selected from halo, hydroxyl, C₁₋₄ alkyl, C₁₋₄ alkoxyl, and —(C₁₋₄ alkylene-C(O)N(R⁵)(R⁶))
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

### Moiety for BTK

In certain embodiments, the TPL is a moiety that binds to Bruton's tyrosine kinase (BTK). Exemplary compounds that bind to BTK are reported in the literature, such as ibrutinib and zanubrutinib. A radical of such compounds reported in the literature that bind BTK are amenable for use in the present invention.

Exemplary ompounds that inhibit BTKI that are reported in the literature include:

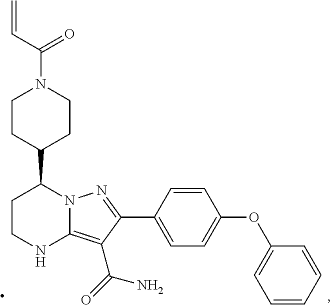 [SMILES: C=CC(=O)N1CCC([C@@H]2CCNc3c(C(N)=O)c(-c4ccc(Oc5ccccc5)cc4)nn32)CC1]

as described in Guo, Y. et al., in J Med Chem 2019, 62(17): 7923.

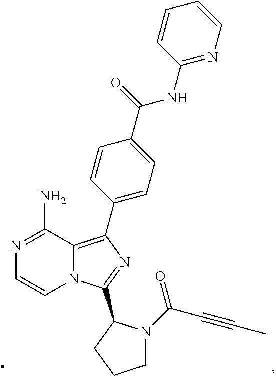 [SMILES: CC#CC(=O)N1CCC[C@H]1c1nc(-c2ccc(C(=O)Nc3ccccn3)cc2)c2c(N)nccn12]

as described in Hopper, M. Et al in J Pharmacol Exp Ther 2020, 372(3): 331.

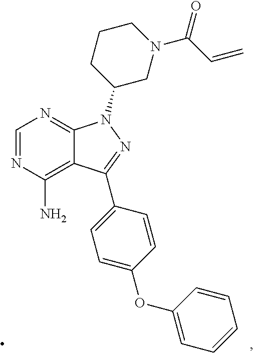 [SMILES: C=CC(=O)N1CCC[C@@H](n2nc(-c3ccc(Oc4ccccc4)cc3)c3c(N)ncnc32)C1]

as described in Honigbert, L. Et al in WO 2008/039218

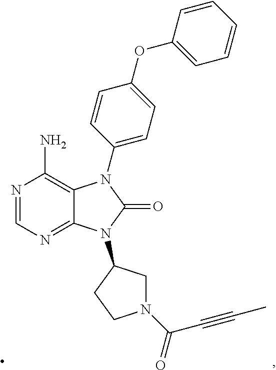 [SMILES: CC#CC(=O)N1CC[C@@H](n2c(=O)n(-c3ccc(Oc4ccccc4)cc3)c3c(N)ncnc32)C1]

as described in Yamamoto, S. Et al in WO 2011/152351

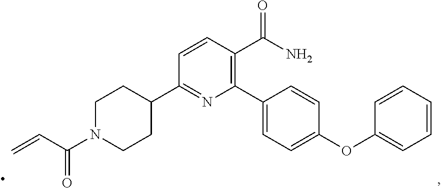 [SMILES: C=CC(=O)N1CCC(c2ccc(C(N)=O)c(-c3ccc(Oc4ccccc4)cc3)n2)CC1]

as described in Chen, X. Et al in WO 2015/048662

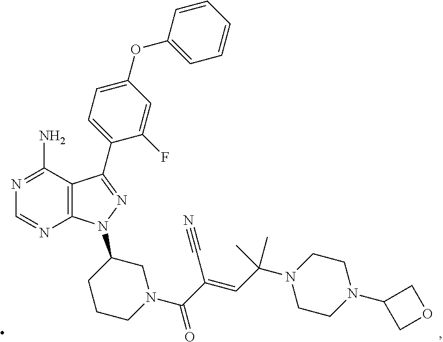 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

as described in Owens, T. Et al in WO 2014/039899

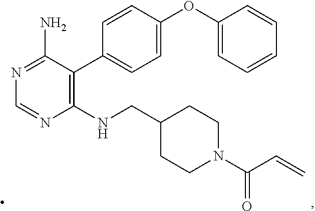 [SMILES: C=CC(=O)N1CCC(CNc2ncnc(N)c2-c2ccc(Oc3ccccc3)cc2)CC1]

as described in Caldwell, R. Et al in J Med Chem 2019, 62(17):

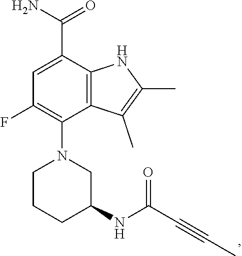 [SMILES: CC#CC(=O)N[C@H]1CCCN(c2c(F)cc(C(N)=O)c3[nH]c(C)c(C)c23)C1]

as described in Watterson, S. Et al in J Med Chem 2019, 62(7): 3228

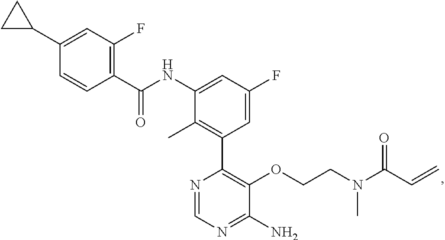 [SMILES: C=CC(=O)N(C)CCOc1c(N)ncnc1-c1cc(F)cc(NC(=O)c2ccc(C3CC3)cc2F)c1C]

as described in Angst, D. Et al in WO 2015/079417

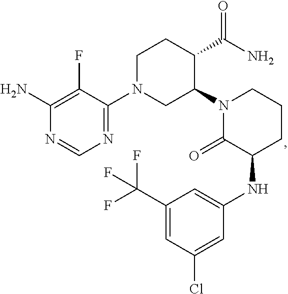 [SMILES: NC(=O)[C@H]1CCN(c2ncnc(N)c2F)C[C@@H]1N1CCC[C@@H](Nc2cc(Cl)cc(C(F)(F)F)c2)C1=O]

as described in Hopkins, B. Et al in WO 2013/185084.

In certain embodiments, the TPL is a radical of one of the above compounds, which is attached to L² through a modifiable oxygen, nitrogen, or carbon atom.

In certain embodiments, the TPL is one of the following:

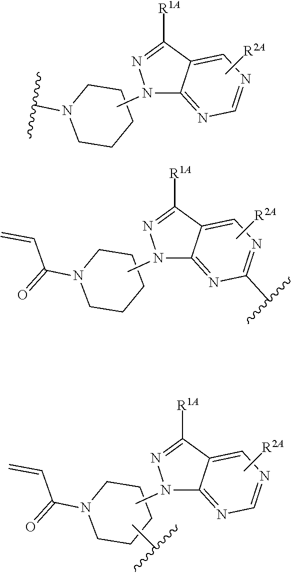

wherein:
R^{1A} is -(phenyl optionally substituted with 1, 2, 3, or 5 substituents independently selected from halo, hydroxyl, C₁₋₄ alkyl, and C₁₋₄ alkoxyl)-O-(phenyl optionally substituted with 1, 2, 3, or 5 substituents independently selected from halo, hydroxyl, C₁₋₄ alkyl, and C₁₋₄ alkoxyl);
R^{2A} is hydrogen, halo, hydroxyl, C₁₋₄ alkyl, C₁₋₄ alkoxyl, or —N(R^{5A})(R^{6A}); and R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is the following:

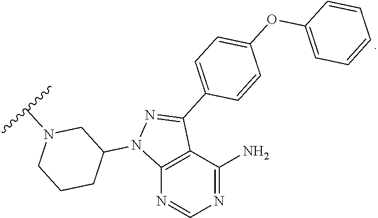 [SMILES: *N1CCCC(n2nc(-c3ccc(Oc4ccccc4)cc3)c3c(N)ncnc32)C1]

In certain embodiments, the TPL is one of the following:

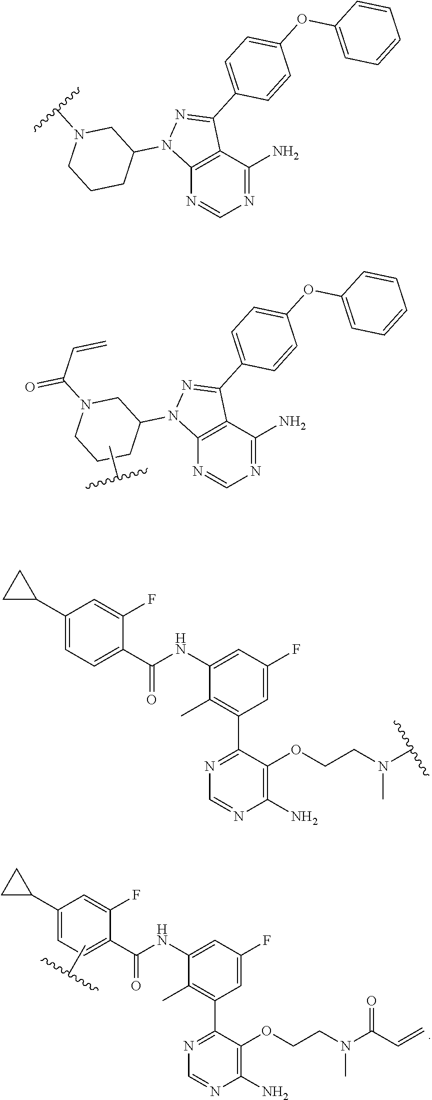

### Moiety for EGFR

In certain embodiments, the TPL is a moiety that binds to or inhibits epidermal growth factor receptor (EGFR). Exemplary compounds that bind to and/or inhibit EGFR are reported in the literature, such as Osimertinib and mavelertinib. A radical of such compounds reported in the literature that bind EGFR are amenable for use in the present invention. In certain embodiments, the TPL is a moiety that binds to EGFR.

Exemplary compounds that inhibit or bind to EGFR that are reported in the literature include:

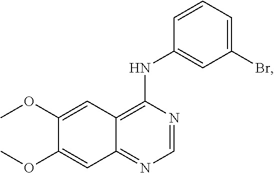 [SMILES: COc1cc2ncnc(Nc3cccc(Br)c3)c2cc1OC]

as described in Gangjee, A. et al., WO 2012/106522;

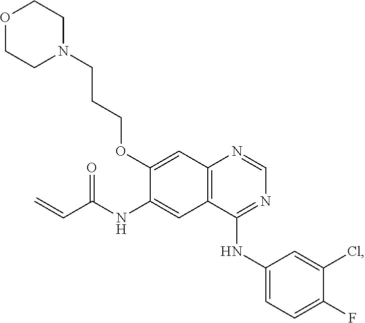 [SMILES: C=CC(=O)Nc1cc2c(Nc3ccc(F)c(Cl)c3)ncnc2cc1OCCCN1CCOCC1]

as described in Huang, Z. et al., WO 2012/027960;

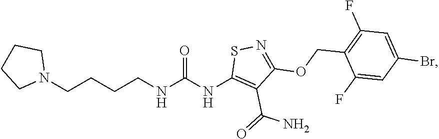 [SMILES: NC(=O)c1c(OCc2c(F)cc(Br)cc2F)nsc1NC(=O)NCCCCN1CCCC1]

as described in Bingaman, D. P. et al., WO 2014/152661;

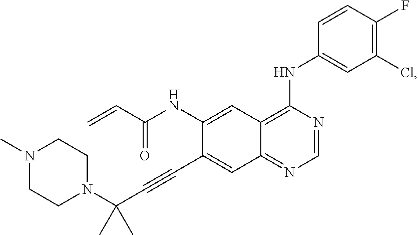 [SMILES: C=CC(=O)Nc1cc2c(Nc3ccc(F)c(Cl)c3)ncnc2cc1C#CC(C)(C)N1CCN(C)CC1]

as described in Kitano, Y. et al., WO 2002/066445;

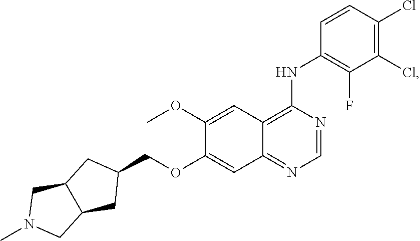 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

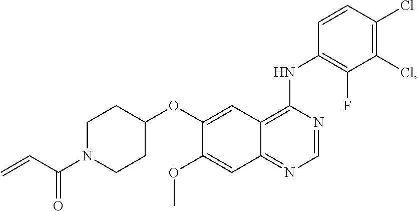 [SMILES: C=CC(=O)N1CCC(Oc2cc3c(Nc4ccc(Cl)c(Cl)c4F)ncnc3cc2OC)CC1]

as described in Lee, K.-O. et al., WO 2008/150118;

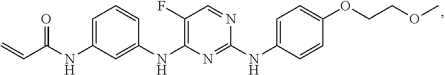 [SMILES: C=CC(=O)Nc1cccc(Nc2nc(Nc3ccc(OCCOC)cc3)ncc2F)c1]

as described in Kluge, A. F. et al., WO 2009/158571;

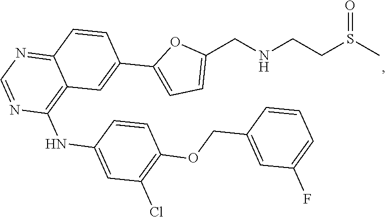 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

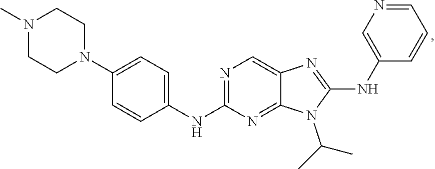 [SMILES: CC(C)n1c(Nc2cccnc2)nc2cnc(Nc3ccc(N4CCN(C)CC4)cc3)nc21]

as described in Yang, S. et al., WO 2011/147066;

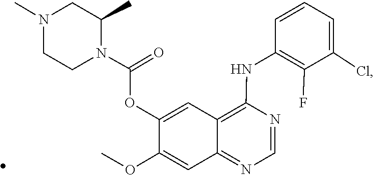 [SMILES: COc1cc2ncnc(Nc3cccc(Cl)c3F)c2cc1OC(=O)N1CCN(C)C[C@H]1C]

as described in Li, D. Y. et al., WO 2014/135876;

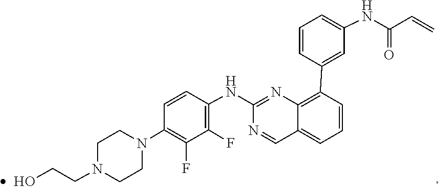 [SMILES: C=CC(=O)Nc1cccc(-c2cccc3cnc(Nc4ccc(N5CCN(CCO)CC5)c(F)c4F)nc23)c1]

as described in Qian, X. et al., WO 2015/027222

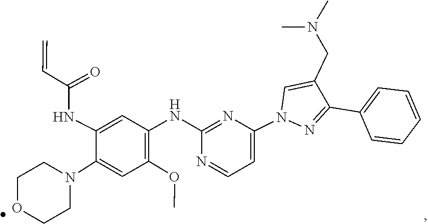 [SMILES: C=CC(=O)Nc1cc(Nc2nccc(-n3cc(CN(C)C)c(-c4ccccc4)n3)n2)c(OC)cc1N1CCOCC1]

as described in Suh, B.-C. et al., WO 2016/060443;

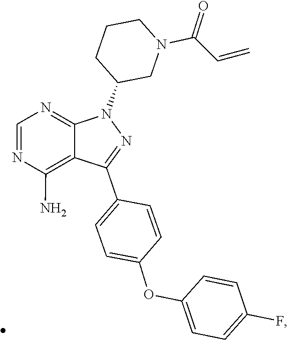 [SMILES: C=CC(=O)N1CCC[C@@H](n2nc(-c3ccc(Oc4ccc(F)cc4)cc3)c3c(N)ncnc32)C1]

as described in Zhang, D. et al., WO 2014/187319;

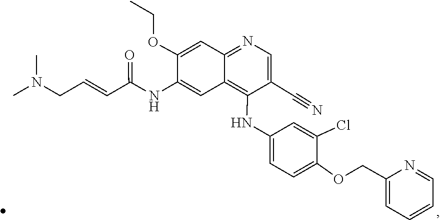 [SMILES: CCOc1cc2ncc(C#N)c(Nc3ccc(OCc4ccccn4)c(Cl)c3)c2cc1NC(=O)/C=C/CN(C)C]

as described in Zhang, D. et al., WO 2015/117547;

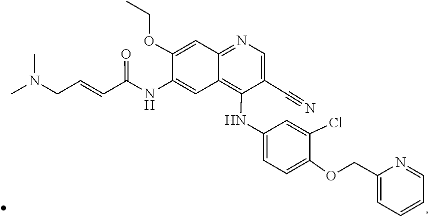 [SMILES: CCOc1cc2ncc(C#N)c(Nc3ccc(OCc4ccccn4)c(Cl)c3)c2cc1NC(=O)/C=C/CN(C)C]

as described in Wissner, A. et al., WO 2005/059678;

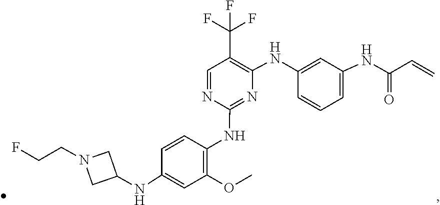 [SMILES: C=CC(=O)Nc1cccc(Nc2nc(Nc3ccc(NC4CN(CCF)C4)cc3OC)ncc2C(F)(F)F)c1]

as described in Lee, K. et al., WO 2012/064706.

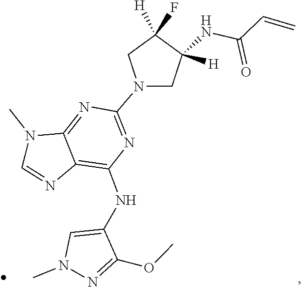 [SMILES: C=CC(=O)N[C@@H]1CN(c2nc(Nc3cn(C)nc3OC)c3ncn(C)c3n2)C[C@H]1F]

as described in Behenna, D. et al., WO 2015/075598.

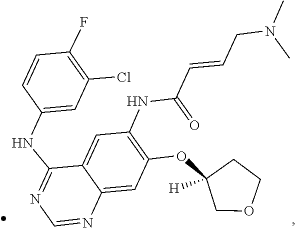 [SMILES: CN(C)C/C=C/C(=O)Nc1cc2c(Nc3ccc(F)c(Cl)c3)ncnc2cc1O[C@H]1CCOC1]

as described in Himmelsbach, F. et al., WO 2002/050043.

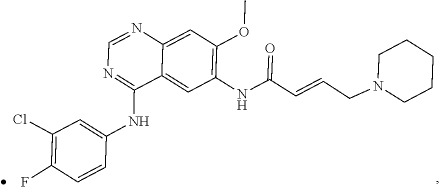 [SMILES: COc1cc2ncnc(Nc3ccc(F)c(Cl)c3)c2cc1NC(=O)/C=C/CN1CCCCC1]

as described in Fakhoury, S. et al., WO 2005/107758.

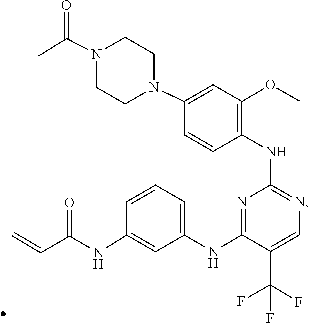 [SMILES: C=CC(=O)Nc1cccc(Nc2nc(Nc3ccc(N4CCN(C(C)=O)CC4)cc3OC)ncc2C(F)(F)F)c1]

as described in Lee, K. et al., WO 2012/061299.

In certain embodiments, the TPL is a radical of one of the above compounds, which is attached to L² through a modifiable oxygen, nitrogen, or carbon atom.

In certain embodiments, the TPL is one of the following:

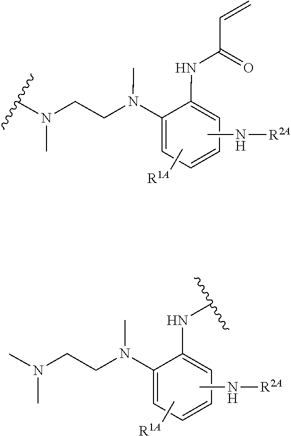

wherein:
R^{1A} is hydrogen, halo, hydroxyl, C₁₋₄ alkyl, C₁₋₄ alkoxyl, or N(R^{5A})(R^{6A}); and
R^{2A} is -(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl)-(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

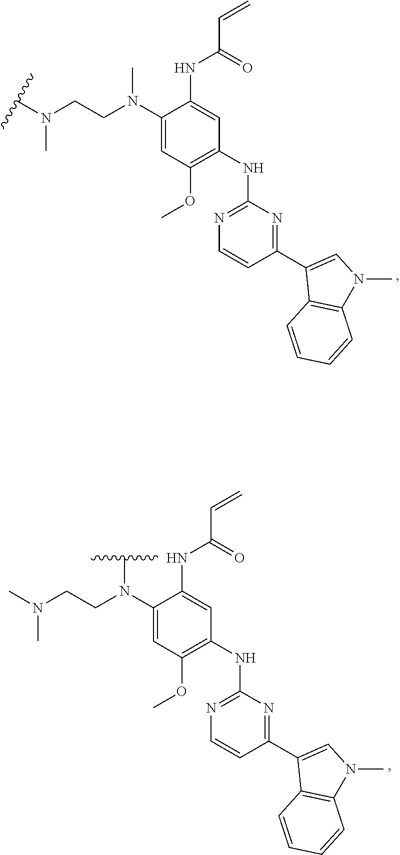

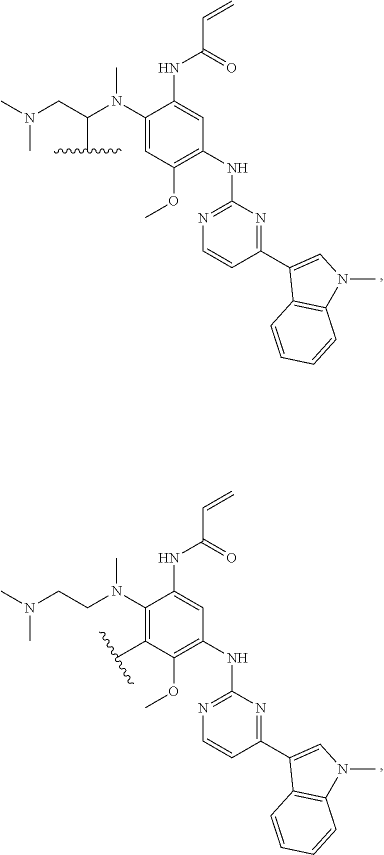

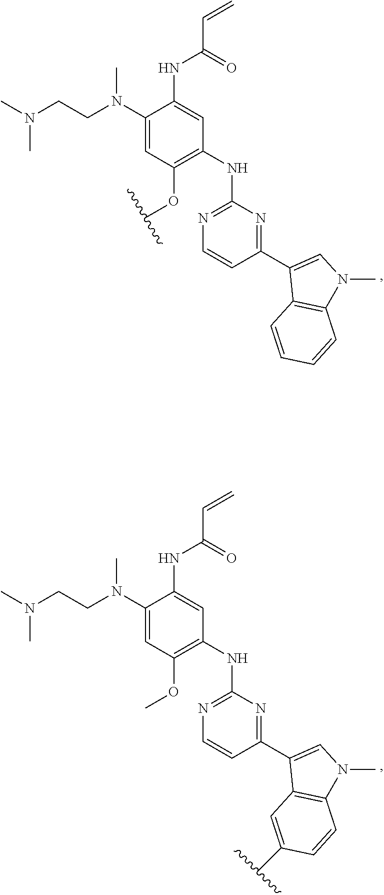

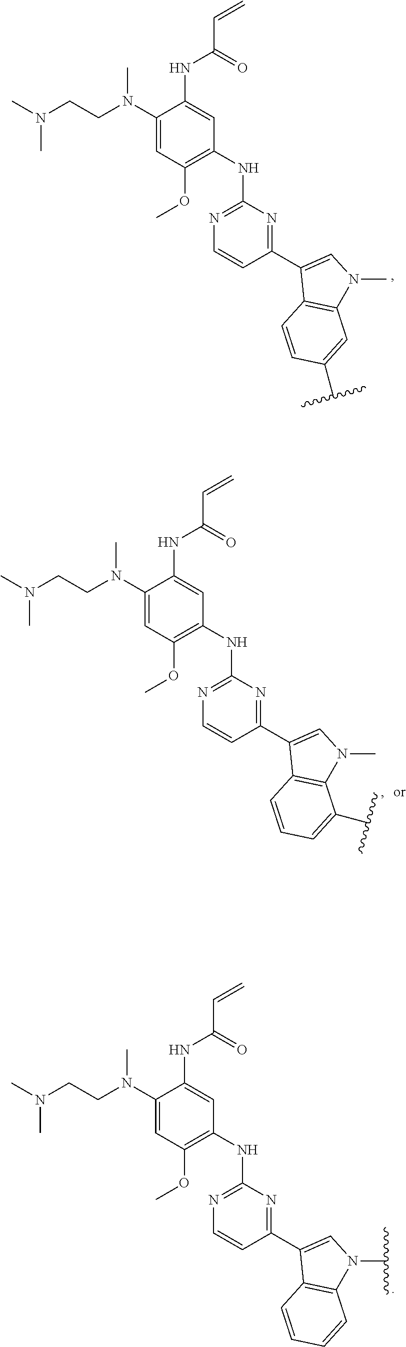

In certain embodiments, the TPL is

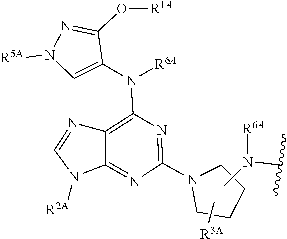

wherein: R^{1A} is C₁₋₄ alkyl; R^{2A} and R^{6A} are independently hydrogen or C₁₋₄ alkyl; R^{3A} is halo; and R^{5A} is C₁₋₆ alkyl or C₃₋₆ cycloalkyl.

In certain embodiments, R^{2A} is hydrogen or C₁₋₄ alkyl. In certain embodiments, R^{2A} is hydrogen. In certain embodiments, R^{2A} is C₁₋₄ alkyl. In certain embodiments, R^{6A} is hydrogen or C₁₋₄ alkyl. In certain embodiments, R^{6A} is hydrogen. In certain embodiments, R^{6A}is C₁₋₄ alkyl.

In certain embodiments, the TPL is one of the following:

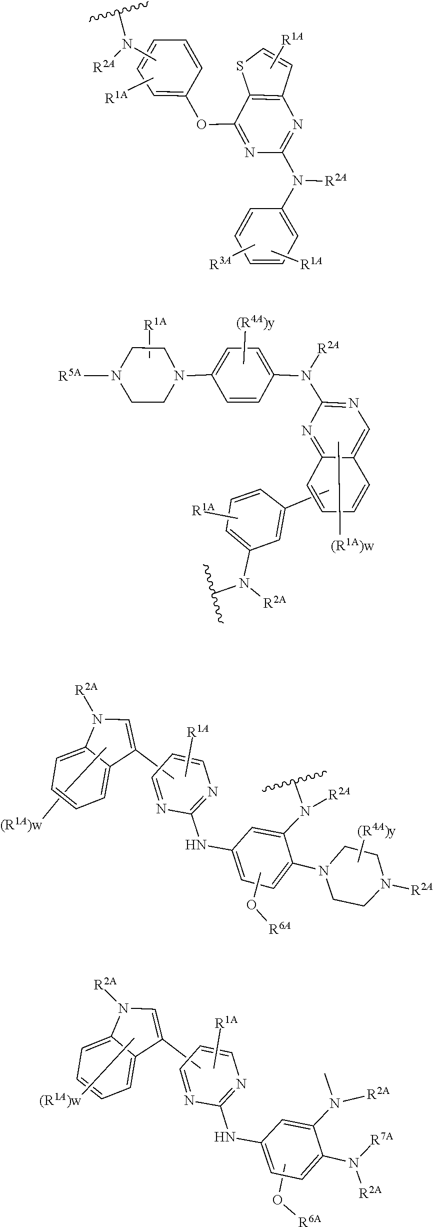

wherein:
R^{1A} and R^{4A} each represent independently for each occurrence hydrogen, halo, hydroxyl, C₁₋₄ alkyl, C₁₋₄ alkoxyl, or C₃₋₆ cycloalkyl;
R^{2A} represents independently for each occurrence hydrogen or C₁₋₄ alkyl;
R^{3A} is a 3-7 membered saturated heterocyclyl containing 1, 2, or 3 heteroatoms independently selected from oxygen, nitrogen, and sulfur, wherein the heterocyclyl is substituted with 0, 1, 2, or 3 substituents independently selected from halo, hydroxyl, C₁₋₄ alkyl, C₁₋₄ alkoxyl, or C₃₋₆ cycloalkyl;
R^{5A} is C₁₋₆ hydroxyalkyl or C₁₋₆ alkyl;
R^{6A} is C₁₋₆ alkyl or C₃₋₆ cycloalkyl;
R^{7A} is C₁₋₆ alkylene)-N(R^{2A})₂;
y and w are independently 1 or 2.

In certain embodiments, the TPL is

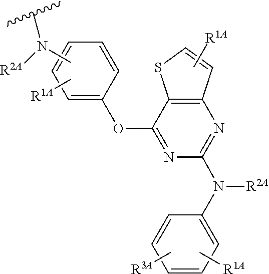

where the variables are as defined above. In certain embodiments, the TPL is

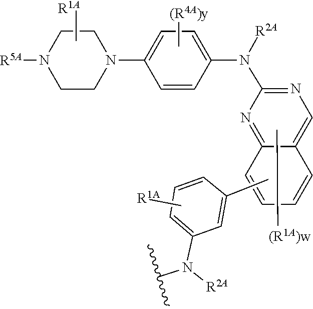

where the variables are as defined above. In certain embodiments, the TPL is

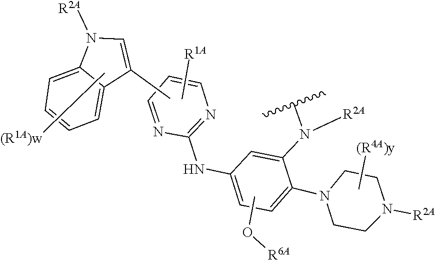

where the variables are as defined above. In certain embodiments, the TPL is

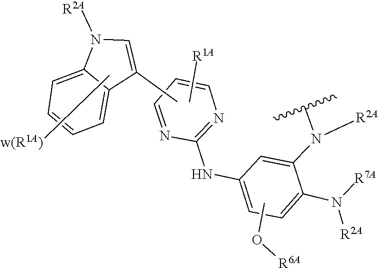

where the variables are as defined above.

In certain embodiments, R^{1A} represents independently for each occurrence hydrogen, halo, hydroxyl, C₁₋₄ alkyl, C₁₋₄ alkoxyl, or C₃₋₆ cycloalkyl. In certain embodiments, R^{1A} is hydrogen. In certain embodiments, R^{1A} is halo. In certain embodiments, R^{1A} is hydroxyl. In certain embodiments, R^{1A} is C₁₋₄ alkyl. In certain embodiments, R^{1A} is C₁₋₄ alkoxyl. In certain embodiments, R^{1A} is C₃₋₆ cycloalkyl. In certain embodiments, R^{4A} represents independently for each occurrence hydrogen, halo, hydroxyl, C₁₋₄ alkyl, C₁₋₄ alkoxyl, or C₃₋₆ cycloalkyl. In certain embodiments, R^{4A} is hydrogen. In certain embodiments, R^{4A} is halo. In certain embodiments, R^{4A} is hydroxyl. In certain embodiments, R^{4A} is C₁₋₄ alkyl. In certain embodiments, R^{4A} is C₁₋₄ alkoxyl. In certain embodiments, R^{4A} is C₃₋₆ cycloalkyl.

In certain embodiments, R^{2A} is hydrogen. In certain embodiments, R^{2A} is C₁₋₄ alkyl. In certain embodiments, R^{3A} is a 5-6 membered saturated heterocyclyl containing 1 or 2 heteroatoms independently selected from oxygen and nitrogen, wherein the heterocyclyl is substituted with 0, 1, 2, or 3 substituents independently selected from halo, hydroxyl, C₁₋₄ alkyl, C₁₋₄ alkoxyl, or C₃₋₆ cycloalkyl. In certain embodiments, R^{3A} is piperazinyl substituted with 0, 1, 2, or 3 substituents independently selected from halo and C₁₋₄ alkyl.

In certain embodiments, R^{5A} is C₁₋₆ hydroxyalkyl. In certain embodiments, R^{5A} is C₁₋₆ alkyl. In certain embodiments, R^{6A} is C₁₋₆ alkyl. In certain embodiments, R^{6A} is C₃₋₆ cycloalkyl.

In certain embodiments, R^{7A} is C₁₋₄ alkylene)-N(R^{2A})₂.

In certain embodiments, y is 1. In certain embodiments, y is 2. In certain embodiments, w is 1. In certain embodiments, w is 2.

In certain embodiments, the TPL is one of the following:

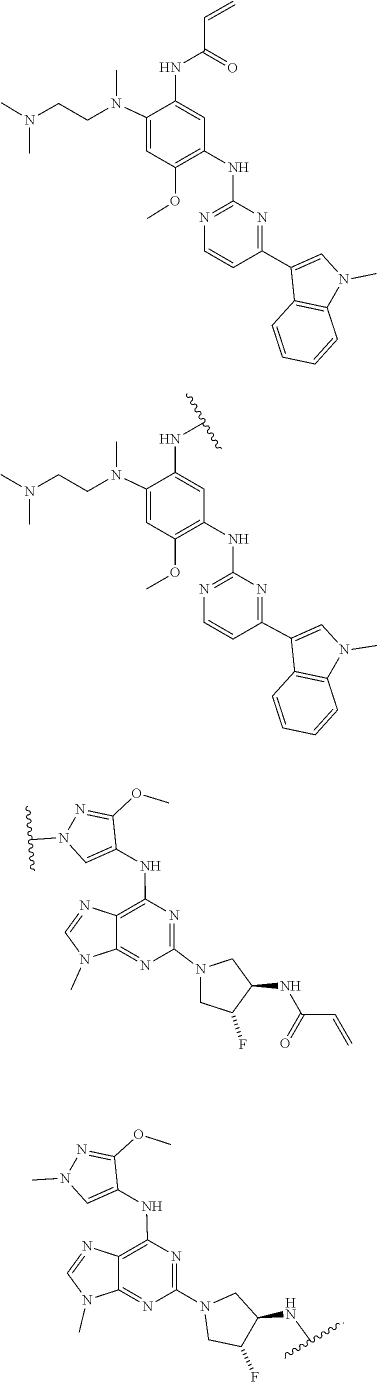

In certain embodiments, the TPL is one of the following:

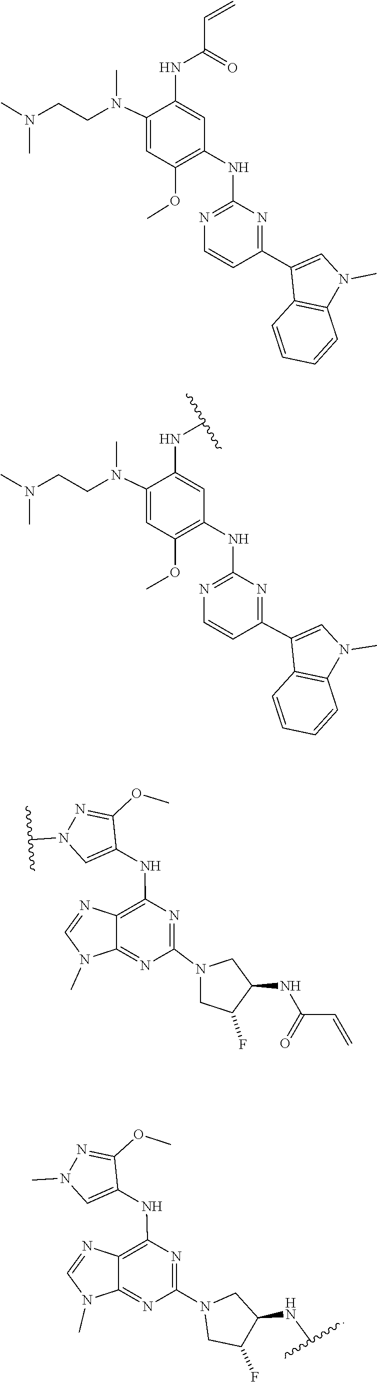

In certain embodiments, the TPL is one of the following:

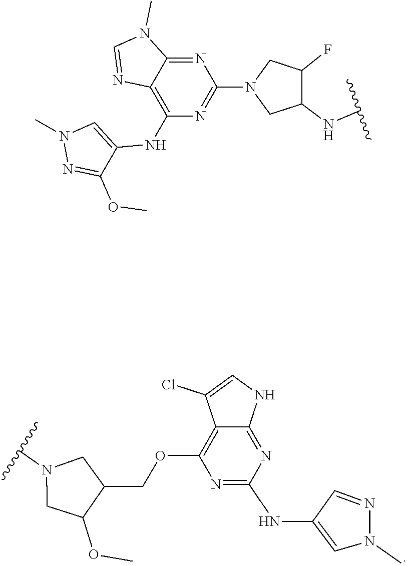 [SMILES: *NC1CN(c2nc(Nc3cn(C)nc3OC)c3ncn(C)c3n2)CC1F ; *N1CC(COc2nc(Nc3cnn(C)c3)nc3[nH]cc(Cl)c23)C(OC)C1]

In certain embodiments, the TPL is one of the following:

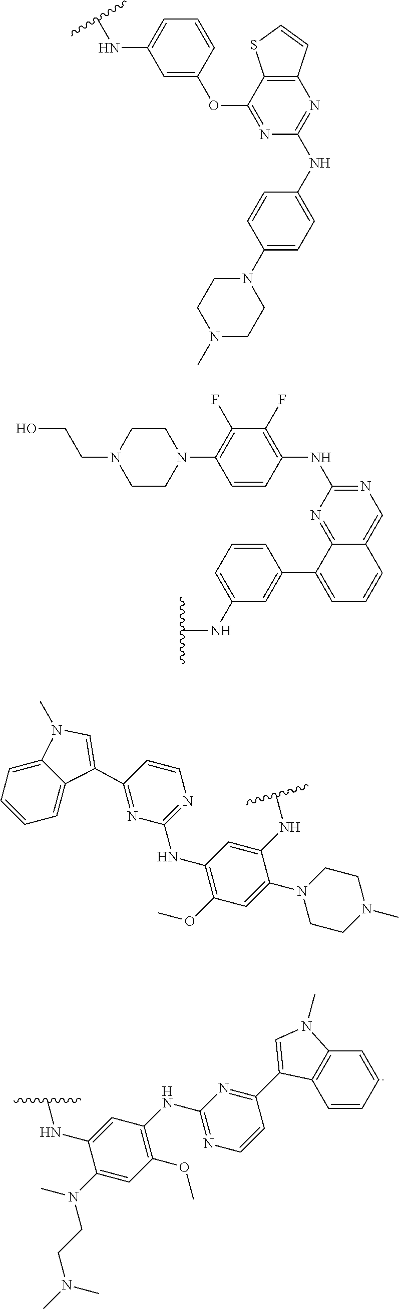

In certain embodiments, the TPL is one of the following:

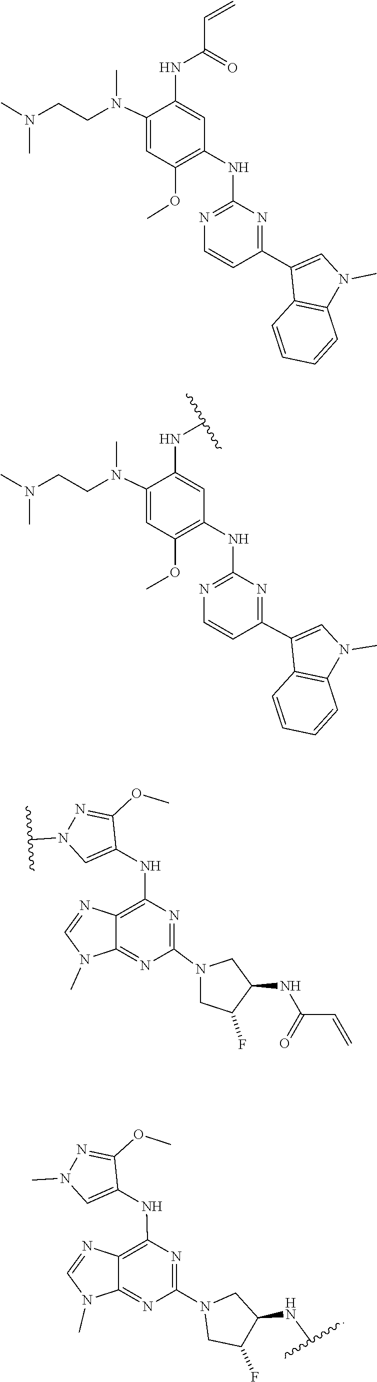

### Moiety for AR

In certain embodiments, the TPL is a moiety that binds to androgen receptor (AR) protein. Exemplary compounds that bind to AR are reported in the literature, such as TMBC and 5N-bicalutamide. A radical of such compounds reported in the literature that bind AR are amenable for use in the present invention.

Exemplary compounds that are agonists of the AR that are reported in the literature include:

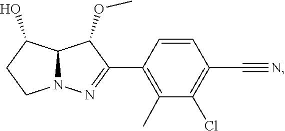 [SMILES: CO[C@@H]1C(c2ccc(C#N)c(Cl)c2C)=NN2CC[C@H](O)[C@@H]12]

as described in Ullrich, T. et al., in WO 2013/014627.

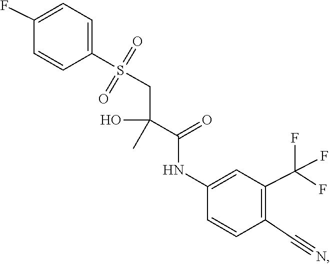 [SMILES: CC(O)(CS(=O)(=O)c1ccc(F)cc1)C(=O)Nc1ccc(C#N)c(C(F)(F)F)c1]

as described in Yoshino, H. et al., in Bioorg Med Chem 2010, 18(23): 8150.

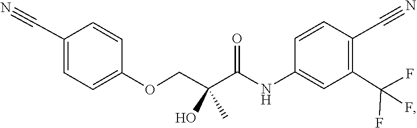 [SMILES: C[C@](O)(COc1ccc(C#N)cc1)C(=O)Nc1ccc(C#N)c(C(F)(F)F)c1]

as described in Steinr, M. et al., in US 2016/128968.

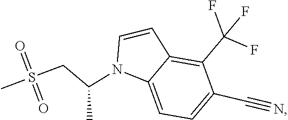 [SMILES: C[C@H](CS(C)(=O)=O)n1ccc2c(C(F)(F)F)c(C#N)ccc21]

as described in Cadilla, R. et al., in 2019/127326.

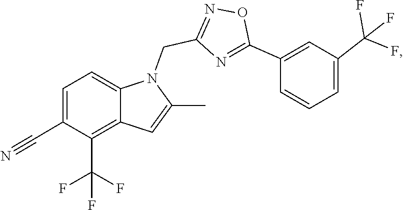 [SMILES: Cc1cc2c(C(F)(F)F)c(C#N)ccc2n1Cc1noc(-c2cccc(C(F)(F)F)c2)n1]

as described in turnbull, P. et al., in 249th Am Chem Soc (ACS) Natl Meet⋅Mar. 22, 2015/Mar. 26, 2015 ⋅Denver, United Sates⋅Abst MEDI 247.

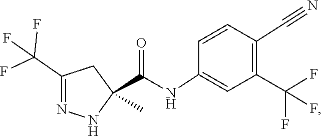 [SMILES: C[C@@]1(C(=O)Nc2ccc(C#N)c(C(F)(F)F)c2)CC(C(F)(F)F)=NN1]

as described in Allan, G. et al., in Endocrine 2007, 32(1): 41.

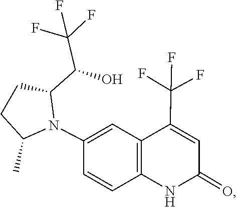 [SMILES: C[C@@H]1CC[C@H]([C@@H](O)C(F)(F)F)N1c1ccc2[nH]c(=O)cc(C(F)(F)F)c2c1]

as described in Martinborough, E. et al., in J Med Chem 2007, 50(21): 5049.

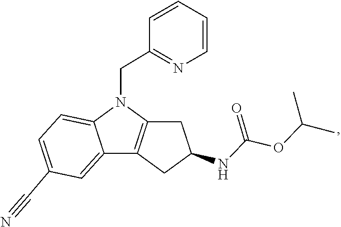 [SMILES: CC(C)OC(=O)N[C@H]1Cc2c(n(Cc3ccccn3)c3ccc(C#N)cc23)C1]

as described in Benson, C. et al., in WO 2016/040234.

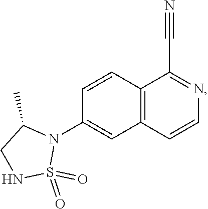 [SMILES: C[C@H]1CNS(=O)(=O)N1c1ccc2c(C#N)nccc2c1]

as described in Chekler, E. et al., in 246th Am Chem Soc (ACS) Natl Meet⋅Sept. 8, 2013/Sep. 12, 2013⋅Indianapolis, United States⋅Abst MEDI 30.

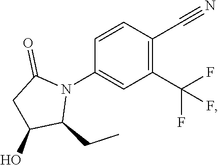 [SMILES: CC[C@H]1[C@@H](O)CC(=O)N1c1ccc(C#N)c(C(F)(F)F)c1]

as described in Aikawa, K. et al., in Bioorg Med Chem 2017, 25(13): 3330.

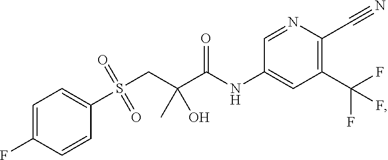 [SMILES: CC(O)(CS(=O)(=O)c1ccc(F)cc1)C(=O)Nc1cnc(C#N)c(C(F)(F)F)c1]

as described in Cortez F. et al., in ACS Chem. Biol. 2017, 12(12): 2934.

In certain embodiments, the TPL is a radical of one of the above compounds, which is attached to L² through a modifiable oxygen, nitrogen, or carbon atom.

In certain embodiments, the TPL is a moiety that is an antagonist of the androgen receptor (AR). Compounds that are an antagonist of the AR are reported in the literature, which include:

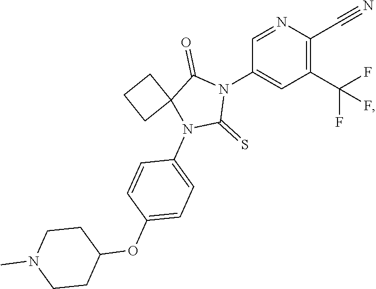 [SMILES: CN1CCC(Oc2ccc(N3C(=S)N(c4cnc(C#N)c(C(F)(F)F)c4)C(=O)C34CCC4)cc2)CC1]

as described in Bignan, G. et al., in WO 2018/009694.

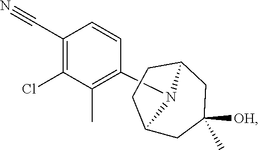 [SMILES: Cc1c(N2[C@@H]3CC[C@H]2C[C@@](C)(O)C3)ccc(C#N)c1Cl]

as described in Sunden, H. et al., in J Med Chem 2015, 58(3): 1569.

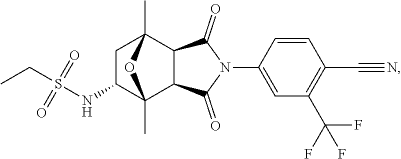 [SMILES: CCS(=O)(=O)N[C@@H]1C[C@@]2(C)O[C@]1(C)[C@@H]1C(=O)N(c3ccc(C#N)c(C(F)(F)F)c3)C(=O)[C@@H]12]

as described in Balog, A. et al., in ACS Med Chem Lett 2015, 6(8): 908.

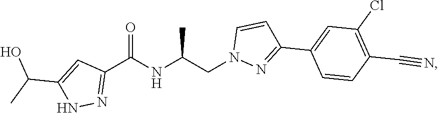 [SMILES: CC(O)c1cc(C(=O)N[C@@H](C)Cn2ccc(-c3ccc(C#N)c(Cl)c3)n2)n[nH]1]

as described in Sugawara, T. et al., in Int J Cancer 2019, 145(5): 1382.

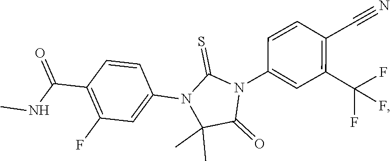 [SMILES: CNC(=O)c1ccc(N2C(=S)N(c3ccc(C#N)c(C(F)(F)F)c3)C(=O)C2(C)C)cc1F]

as described in Sawyers, C. et al., in WO 2006/124118

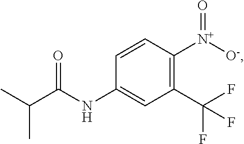

as described in Rizner, T. et al., in Steroids 2011, 76(6): 607.

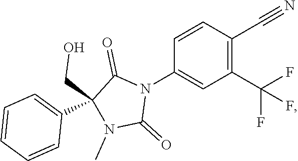 [SMILES: CN1C(=O)N(c2ccc(C#N)c(C(F)(F)F)c2)C(=O)[C@@]1(CO)c1ccccc1]

as described in Sabchareon, A. et al., in J Med Chem 2012, 55(19): 8236.

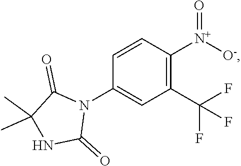

as described in Schlienger, N. et al., in J Med Chem 2009, 52(22): 7186.

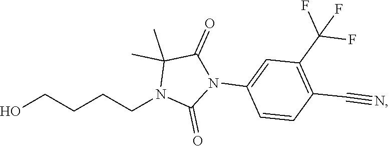 [SMILES: CC1(C)C(=O)N(c2ccc(C#N)c(C(F)(F)F)c2)C(=O)N1CCCCO]

as described in Huang, T. et al., in J Med Chem 2010, 53(11): 4422.

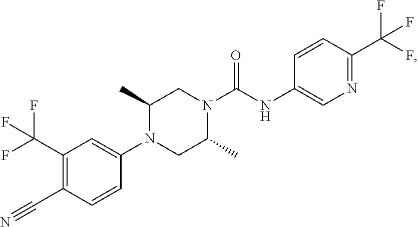 [SMILES: C[C@@H]1CN(c2ccc(C#N)c(C(F)(F)F)c2)[C@@H](C)CN1C(=O)Nc1ccc(C(F)(F)F)nc1]

as described in Kinoyama, I. et al., in J Med Chem 2006, 49(2): 716.

In certain embodiments, the TPL is a radical of one of the above compounds, which is attached to L² through a modifiable oxygen, nitrogen, or carbon atom.

In certain embodiments, the TPL is one of the following:

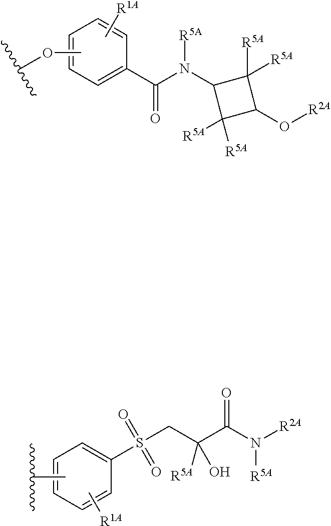

wherein:
R^{1A} is hydrogen, halo, hydroxyl, C₁₋₄ alkyl, C₁₋₄ alkoxyl, or —N(R^{8A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄haloalkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one following:

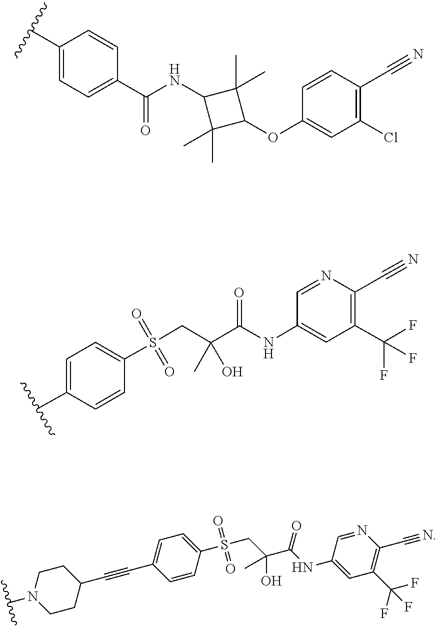 [SMILES: *c1ccc(C(=O)NC2C(C)(C)C(Oc3ccc(C#N)c(Cl)c3)C2(C)C)cc1 ; *c1ccc(S(=O)(=O)CC(C)(O)C(=O)Nc2cnc(C#N)c(C(F)(F)F)c2)cc1 ; *N1CCC(C#Cc2ccc(S(=O)(=O)CC(C)(O)C(=O)Nc3cnc(C#N)c(C(F)(F)F)c3)cc2)CC1]

In certain embodiments, the TPL is or

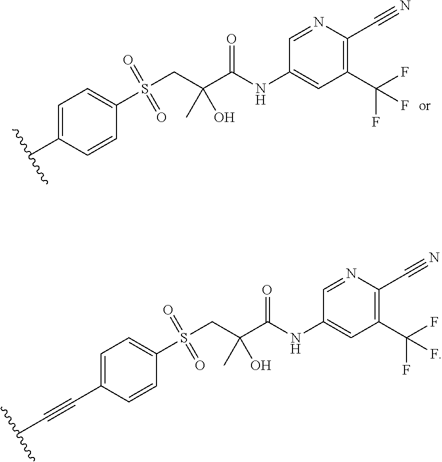 [SMILES: *c1ccc(S(=O)(=O)CC(C)(O)C(=O)Nc2cnc(C#N)c(C(F)(F)F)c2)cc1 ; *C#Cc1ccc(S(=O)(=O)CC(C)(O)C(=O)Nc2cnc(C#N)c(C(F)(F)F)c2)cc1]

### Moiety for IDH1

In certain embodiments, the TPL is a moiety that binds to IDH1. Exemplary compounds that bind to IDH1 are reported in the literature, such as LY3410738. A radical of such compounds reported in the literature that bind IDH1 are amenable for use in the present invention.

In certain embodiments, the TPL is one of the following:

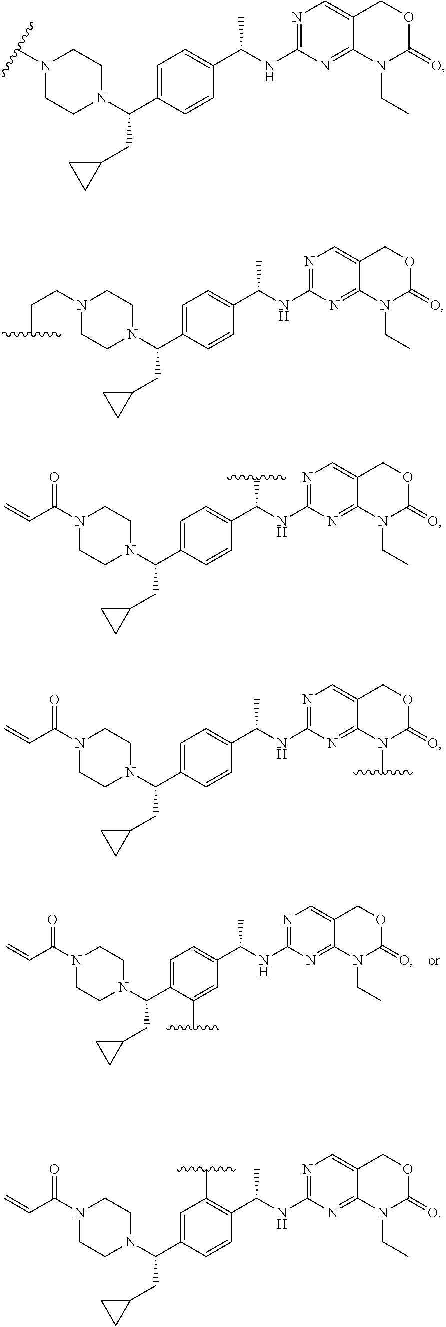 [SMILES: *N1CCN([C@@H](CC2CC2)c2ccc([C@H](C)Nc3ncc4c(n3)N(CC)C(=O)OC4)cc2)CC1 ; *CCN1CCN([C@@H](CC2CC2)c2ccc([C@H](C)Nc3ncc4c(n3)N(CC)C(=O)OC4)cc2)CC1 ; *[C@H](Nc1ncc2c(n1)N(CC)C(=O)OC2)c1ccc([C@H](CC2CC2)N2CCN(C(=O)C=C)CC2)cc1 ; *N1C(=O)OCc2cnc(N[C@@H](C)c3ccc([C@H](CC4CC4)N4CCN(C(=O)C=C)CC4)cc3)nc21 ; *c1cc([C@H](C)Nc2ncc3c(n2)N(CC)C(=O)OC3)ccc1[C@H](CC1CC1)N1CCN(C(=O)C=C)CC1 ; *c1cc([C@H](CC2CC2)N2CCN(C(=O)C=C)CC2)ccc1[C@H](C)Nc1ncc2c(n1)N(CC)C(=O)OC2]

### Moiety for ER

In certain embodiments, the TPL is a moiety that binds to the estrogen receptor (ER). Exemplary compounds that bind to ER are reported in the literature, such as raloxifene, H3B-6545, and AZD9496. A radical of such compounds reported in the literature that bind ER are amenable for use in the present invention.

In certain embodiments, the TPL is a moiety that is an activator, inhibitor, and/or bind to the estrogen receptor (ER). Compounds that activate, inhibit, and/or bind to the ER are reported in the literature, which include:

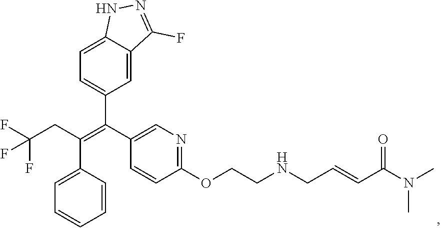 [SMILES: CN(C)C(=O)/C=C/CNCCOc1ccc(/C(=C(/CC(F)(F)F)c2ccccc2)c2ccc3[nH]nc(F)c3c2)cn1]

as described in Bock, M. et al., in US20160347717.

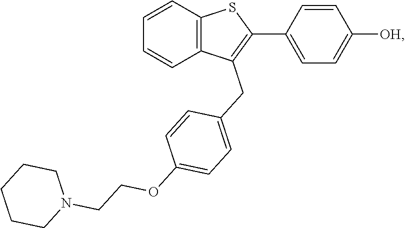 [SMILES: Oc1ccc(-c2sc3ccccc3c2Cc2ccc(OCCN3CCCCC3)cc2)cc1]

as described in Palkowitz, A., in U.S. Pat. No. 5,488,058.

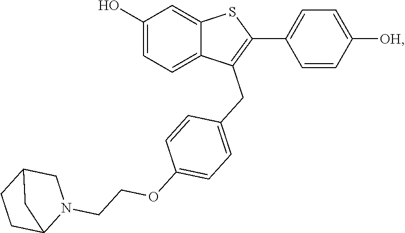 [SMILES: Oc1ccc(-c2sc3cc(O)ccc3c2Cc2ccc(OCCN3CC4CCC3C4)cc2)cc1]

as described in Cameron, K. et al., in WO1995010513.

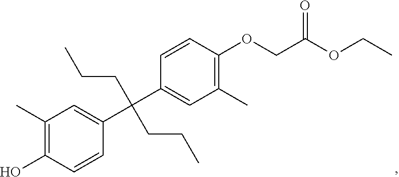 [SMILES: CCCC(CCC)(c1ccc(O)c(C)c1)c1ccc(OCC(=O)OCC)c(C)c1]

as described in Nanjyo, S. et al., in Bioorg Med Chem 2019, 27(10): 1952.

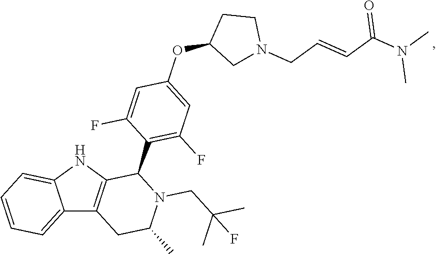 [SMILES: C[C@@H]1Cc2c([nH]c3ccccc23)[C@@H](c2c(F)cc(O[C@H]3CCN(C/C=C/C(=O)N(C)C)C3)cc2F)N1CC(C)(C)F]

as described in Yang, F. et al., in WO2019223715.

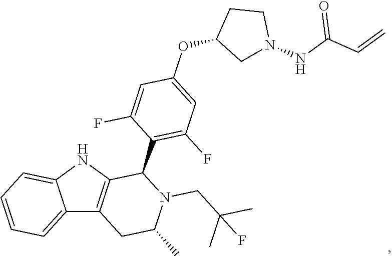

as described in Yang, F. et al., in WO2019223715.

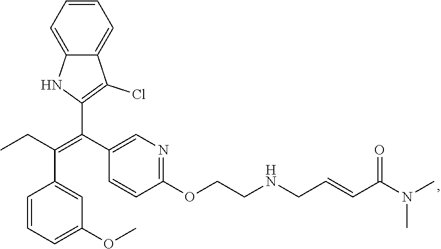 [SMILES: CC/C(=C(/c1ccc(OCCNC/C=C/C(=O)N(C)C)nc1)c1[nH]c2ccccc2c1Cl)c1cccc(OC)c1]

as described in Duan, S. et al., in WO2020125640.

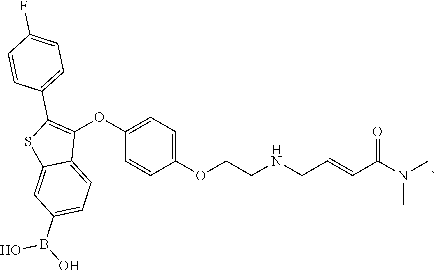 [SMILES: CN(C)C(=O)/C=C/CNCCOc1ccc(Oc2c(-c3ccc(F)cc3)sc3cc(B(O)O)ccc23)cc1]

as described in Wang, G. et al., in WO2020055973.

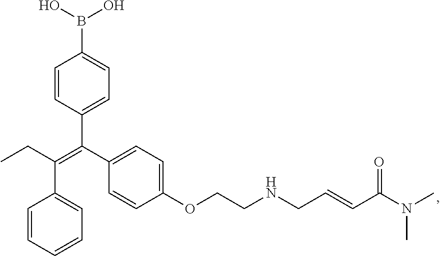 [SMILES: CC/C(=C(/c1ccc(OCCNC/C=C/C(=O)N(C)C)cc1)c1ccc(B(O)O)cc1)c1ccccc1]

as described in Wang, G. et al., in WO2020055973.

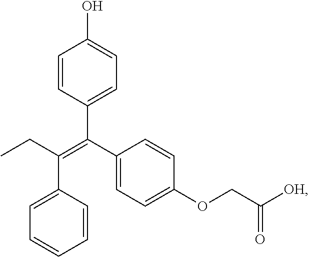 [SMILES: CC/C(=C(\c1ccc(O)cc1)c1ccc(OCC(=O)O)cc1)c1ccccc1]

as described in Ruenitz, P., in 219th Am Chem Soc (ACS) Natl Meet⋅Mar. 26, 2000/Mar. 30, 2000⋅San Francisco, United States⋅Abst MEDI 330.

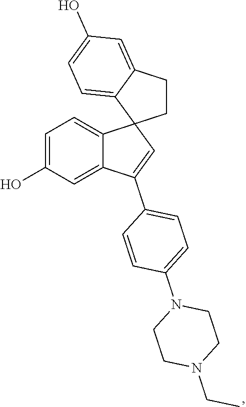 [SMILES: CCN1CCN(c2ccc(C3=CC4(CCc5cc(O)ccc54)c4ccc(O)cc43)cc2)CC1]

as described in Watanabe, N. et al., in Bioorg Med Chem Lett 2003, 13(24): 4317.

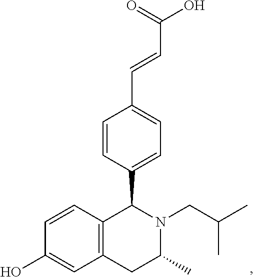 [SMILES: CC(C)CN1[C@H](c2ccc(/C=C/C(=O)O)cc2)c2ccc(O)cc2C[C@H]1C]

as described in Scott, J. et al., in ACS Med Chem Lett 2016, 7(1): 94.

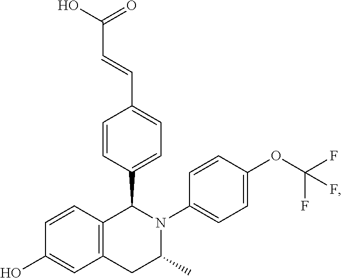 [SMILES: C[C@@H]1Cc2cc(O)ccc2[C@@H](c2ccc(/C=C/C(=O)O)cc2)N1c1ccc(OC(F)(F)F)cc1]

as described in Scott, J. et al., in ACS Med Chem Lett 2016, 7(1): 94.

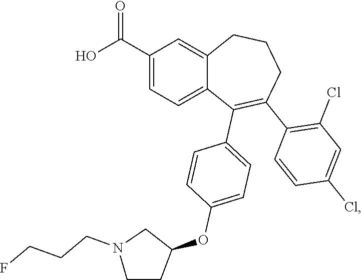 [SMILES: O=C(O)c1ccc2c(c1)CCCC(c1ccc(Cl)cc1Cl)=C2c1ccc(O[C@H]2CCN(CCCF)C2)cc1]

as described in Bouaboula, M. et al., in US2020392081.

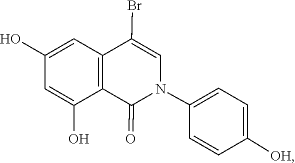 [SMILES: O=c1c2c(O)cc(O)cc2c(Br)cn1-c1ccc(O)cc1]

as described in Dalton, J. et al., in WO2008091555.

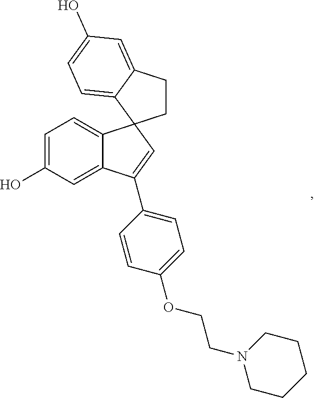 [SMILES: Oc1ccc2c(c1)CCC21C=C(c2ccc(OCCN3CCCCC3)cc2)c2cc(O)ccc21]

as described in Watanabe, N. et al., in J Med Chem 2003, 46(19): 3961.

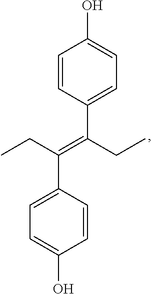 [SMILES: CC/C(=C(/CC)c1ccc(O)cc1)c1ccc(O)cc1]

as described in Nanjyo, S. et al., in Bioorg Med Chem 2019, 27(10): 1952.

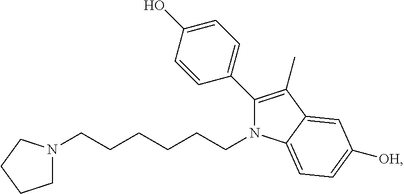 [SMILES: Cc1c(-c2ccc(O)cc2)n(CCCCCCN2CCCC2)c2ccc(O)cc12]

as described in Miller, C. et al., in J Med Chem 2001, 44(11): 1654.

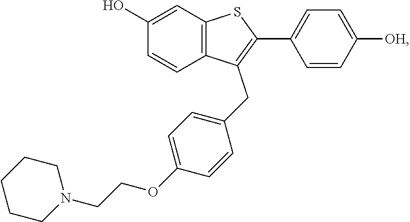 [SMILES: Oc1ccc(-c2sc3cc(O)ccc3c2Cc2ccc(OCCN3CCCCC3)cc2)cc1]

as described in Cameron, K. et al., in WO1995010513.

In certain embodiments, the TPL is a radical of one of the above compounds, which is attached to L² through a modifiable oxygen, nitrogen, or carbon atom.

In certain embodiments, the TPL is

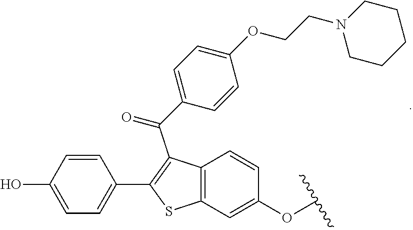 [SMILES: *Oc1ccc2c(C(=O)c3ccc(OCCN4CCCCC4)cc3)c(-c3ccc(O)cc3)sc2c1]

In certain embodiments, the TPL is

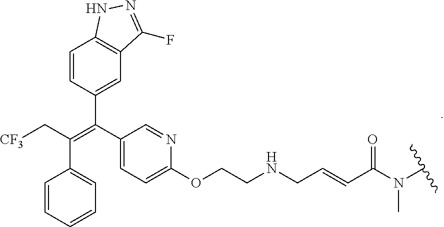 [SMILES: *N(C)C(=O)/C=C/CNCCOc1ccc(/C(=C(/CC(F)(F)F)c2ccccc2)c2ccc3[nH]nc(F)c3c2)cn1]

### Moiety for ALK

In certain embodiments, the TPL is a moiety that binds to anaplastic lymphoma kinase (ALK). Exemplary compounds that bind to ALK are reported in the literature, such as ceritinib. A radical of such compounds reported in the literature that bind ALK are amenable for use in the present invention.

In certain embodiments, the TPL is one of the following:

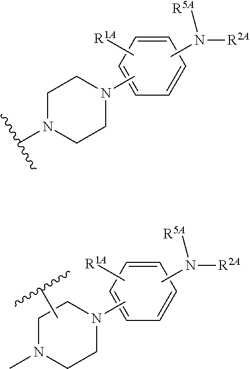

wherein:
R^{1A} is hydrogen, halo, hydroxyl, C₁₋₄ alkyl, C₁₋₄ alkoxyl, or N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl)-N(R^{5A})-(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄haloalkyl, C₁₋₄ alkoxyl, and —P(O)(C₁₋₆ alkyl)₂; and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

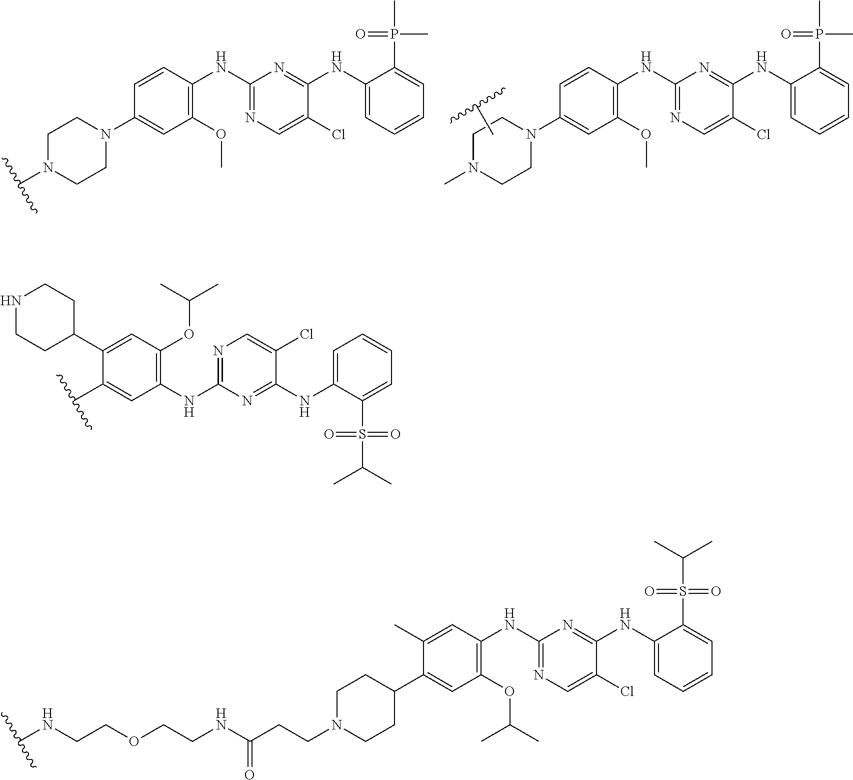

In certain embodiments, the TPL is

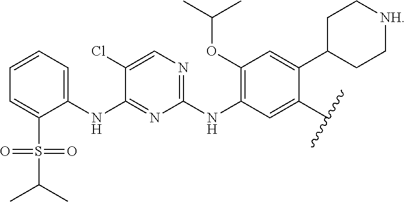 [SMILES: *c1cc(Nc2ncc(Cl)c(Nc3ccccc3S(=O)(=O)C(C)C)n2)c(OC(C)C)cc1C1CCNCC1]

In certain embodiments, the TPL is a moiety that binds to and/or inhibits ALK. Compounds that bind and/or inhibit ALK are reported in the literature, which include EML4. Additional exemplary compounds that bind to and/or inhibit ALK and/or an ALK-fusion protein include:

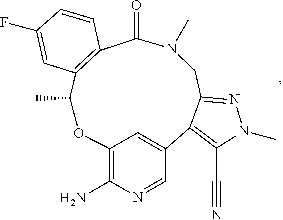 [SMILES: C[C@H]1Oc2cc(cnc2N)-c2c(nn(C)c2C#N)CN(C)C(=O)c2ccc(F)cc21]

as described in Wang, Y., et al., WO2017148325;

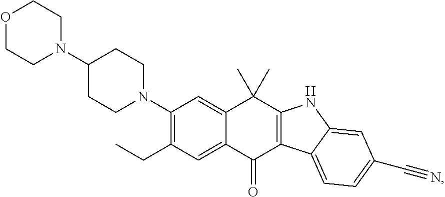 [SMILES: CCc1cc2c(cc1N1CCC(N3CCOCC3)CC1)C(C)(C)c1[nH]c3cc(C#N)ccc3c1C2=O]

as described in Kodama, T., et al., Mol Cancer Ther 2014, 13(12): 2910;

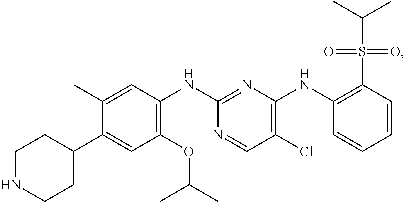 [SMILES: Cc1cc(Nc2ncc(Cl)c(Nc3ccccc3S(=O)(=O)C(C)C)n2)c(OC(C)C)cc1C1CCNCC1]

as described in Marsilje, T. H., et al., J Med Chem. 2013 Jul. 25; 56(14):5675-90 and Chen, J., et al., J Med Chem. 2013 Jul. 25; 56(14):5673-4;

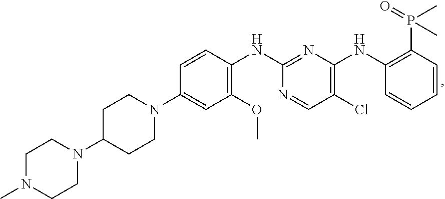

as described in Zhang, S., et al., Clin Cancer Res. 2016 Nov. 15; 22(22):5527-5538;

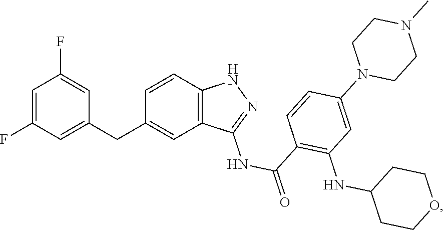 [SMILES: CN1CCN(c2ccc(C(=O)Nc3n[nH]c4ccc(Cc5cc(F)cc(F)c5)cc34)c(NC3CCOCC3)c2)CC1]

as described in Ardini, E., et al., Mol Cancer Ther. 2016 April; 15(4):628-39;

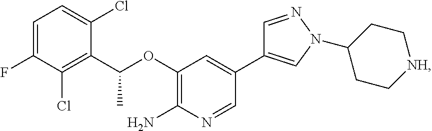 [SMILES: C[C@@H](Oc1cc(-c2cnn(C3CCNCC3)c2)cnc1N)c1c(Cl)ccc(F)c1Cl]

as described in Cui, J. J., et al., J Med Chem. 2011 Sep. 22; 54(18):6342-63;

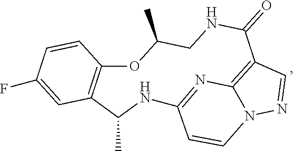 [SMILES: C[C@H]1CNC(=O)c2cnn3ccc(nc23)N[C@H](C)c2cc(F)ccc2O1]

as described in Zhai, D., et al., 108th Annu Meet Am Assoc Cancer Res (AACR)⋅Apr. 1, 2017/Apr. 5, 2017⋅Washington, D.C., United States ⋅Abst 3161, Cancer Res 2017, 77(13);

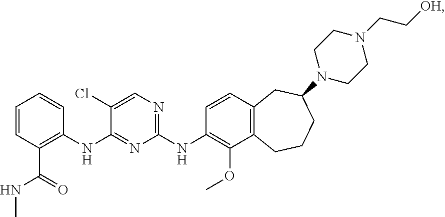 [SMILES: CNC(=O)c1ccccc1Nc1nc(Nc2ccc3c(c2OC)CCC[C@H](N2CCN(CCO)CC2)C3)ncc1Cl]

as described in acobs, Martin, J., et al., WO2013134353;

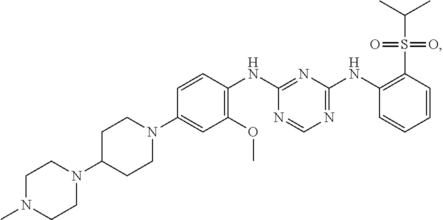 [SMILES: COc1cc(N2CCC(N3CCN(C)CC3)CC2)ccc1Nc1ncnc(Nc2ccccc2S(=O)(=O)C(C)C)n1]

as described in Mori, M., et al., Mol Cancer Ther 2014, 13(2): 329;

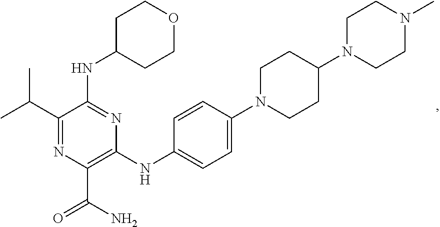 [SMILES: CC(C)c1nc(C(N)=O)c(Nc2ccc(N3CCC(N4CCN(C)CC4)CC3)cc2)nc1NC1CCOCC1]

as described in Shimada, I., et al., WO2010128659;

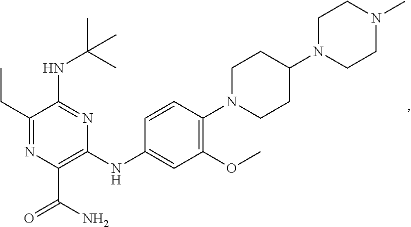 [SMILES: CCc1nc(C(N)=O)c(Nc2ccc(N3CCC(N4CCN(C)CC4)CC3)c(OC)c2)nc1NC(C)(C)C]

as described in Shimada, I., et al., WO2012053606;

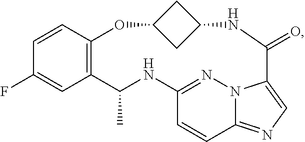 [SMILES: C[C@H]1Nc2ccc3ncc(n3n2)C(=O)N[C@H]2C[C@H](C2)Oc2ccc(F)cc21]

as described in Zhang, Y., et al., WO2019210835;

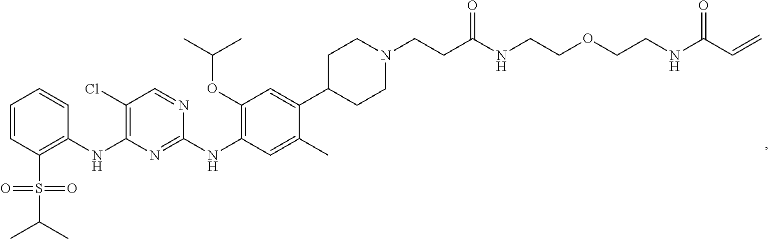 [SMILES: C=CC(=O)NCCOCCNC(=O)CCN1CCC(c2cc(OC(C)C)c(Nc3ncc(Cl)c(Nc4ccccc4S(=O)(=O)C(C)C)n3)cc2C)CC1]

as described in Yan, G., et al., J. Med. Chem 2021, 64(3): 1558.

In certain embodiments, the TPL is a radical of one of the above compounds, which is attached to L² through a modifiable oxygen, nitrogen, or carbon atom.

### Moiety for FGFR1

In certain embodiments, the TPL is a moiety that binds to and/or inhibits Fibroblast Growth Factor Receptor 1 (FGFR1). Compounds that bind and/or inhibit FGFR1 are reported in the literature, which include:

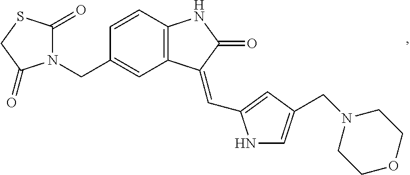 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

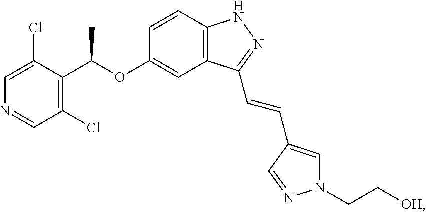 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

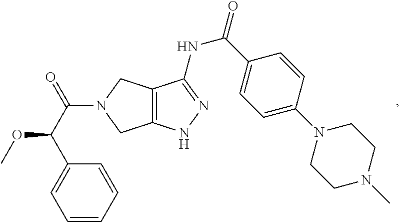

as described in Fancelli, D. et al., J Med Chem 2006, vol. 49(24), page 7247;

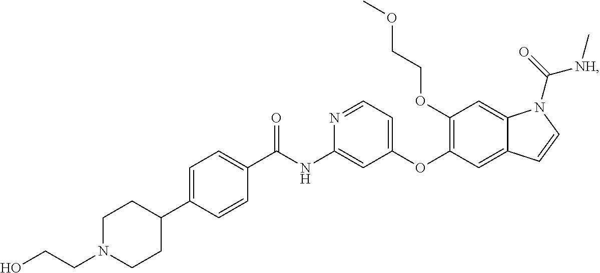 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477;

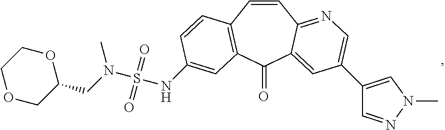 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

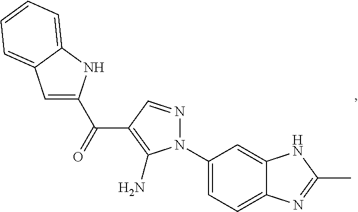 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

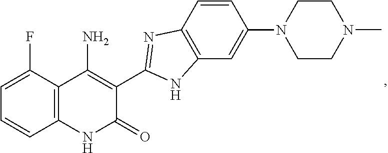 [SMILES: CN1CCN(c2ccc3nc(-c4c(N)c5c(F)cccc5[nH]c4=O)[nH]c3c2)CC1]

as described in Renhowe, P. A. et al., J Med Chem 2009, vol. 52(2), page 278;

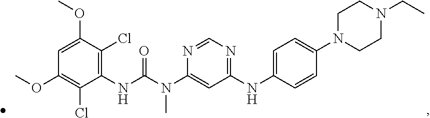 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

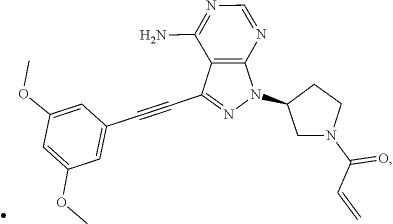 [SMILES: C=CC(=O)N1CC[C@H](n2nc(C#Cc3cc(OC)cc(OC)c3)c3c(N)ncnc32)C1]

as described in Sagara, T. et al., WO 2013/108809;

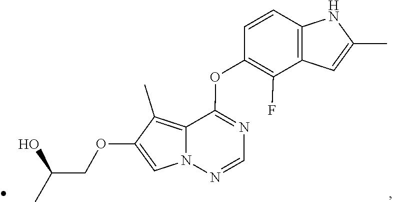 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

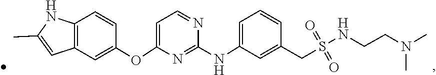

as described in Su, W.-G. et al., WO 2011/060746;

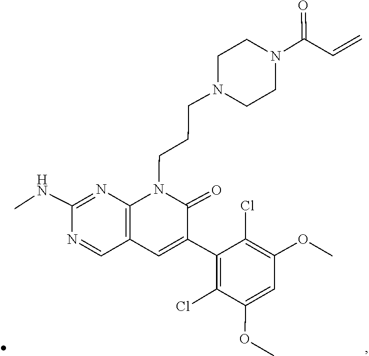 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

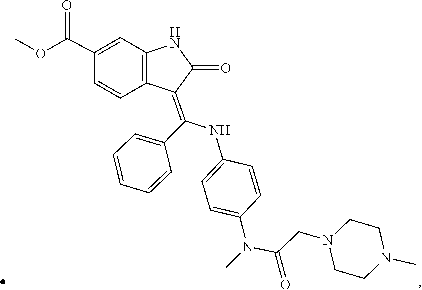

as described in Walters, I. et al., WO 2017/109513;

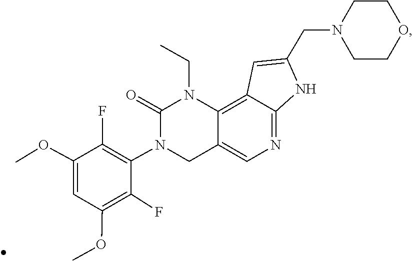 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

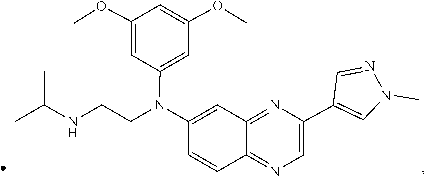 [SMILES: COc1cc(OC)cc(N(CCNC(C)C)c2ccc3ncc(-c4cnn(C)c4)nc3c2)c1]

as described in Xu, X. et al., WO 2018/153373;

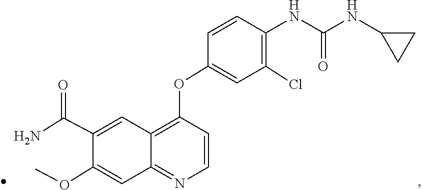 [SMILES: COc1cc2nccc(Oc3ccc(NC(=O)NC4CC4)c(Cl)c3)c2cc1C(N)=O]

as described in Zhang, Y. et al., WO 2019/062637.

In certain embodiments, the TPL is a radical of one of the above compounds, which is attached to L² through a modifiable oxygen, nitrogen, or carbon atom.

### Moiety for FGFR2

In certain embodiments, the TPL is a moiety that binds to and/or inhibits Fibroblast Growth Factor Receptor 2 (FGFR2). Compounds that bind and/or inhibit FGFR2 are reported in the literature, which include:

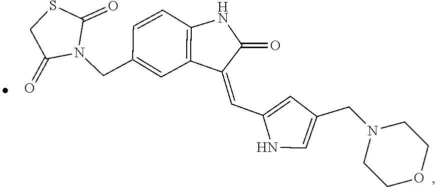 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

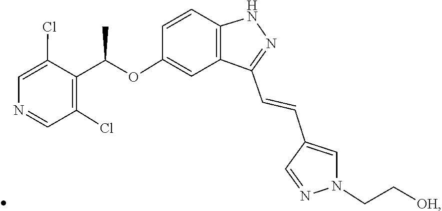 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

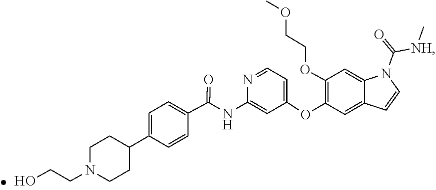 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477;

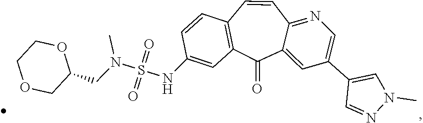 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

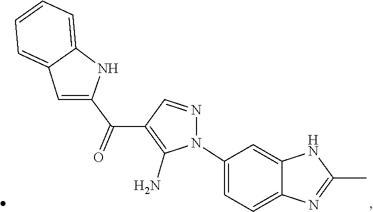 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

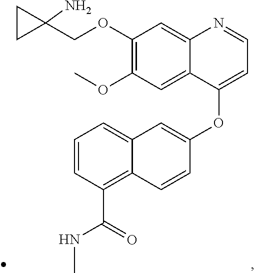 [SMILES: CNC(=O)c1cccc2cc(Oc3ccnc4cc(OCC5(N)CC5)c(OC)cc34)ccc12]

as described in Nguyen, M., et al., 106th Annu Meet Am Assoc Cancer Res (AACR) (April 18-22, Philadelphia) 2015, Abstract 784;

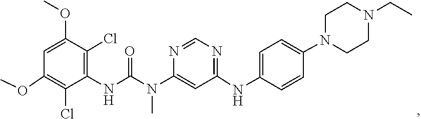 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

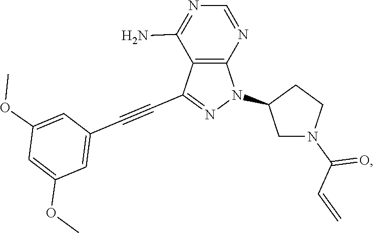 [SMILES: C=CC(=O)N1CC[C@H](n2nc(C#Cc3cc(OC)cc(OC)c3)c3c(N)ncnc32)C1]

as described in Sagara, T. et al., WO 2013/108809;

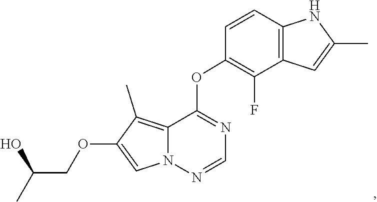 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

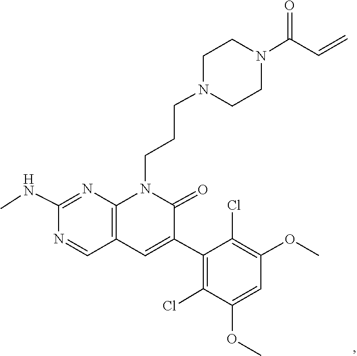 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

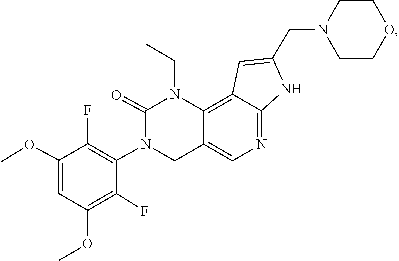 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

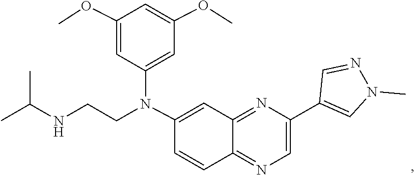 [SMILES: COc1cc(OC)cc(N(CCNC(C)C)c2ccc3ncc(-c4cnn(C)c4)nc3c2)c1]

as described in Xu, X. et al., WO 2018/153373.

In certain embodiments, the TPL is a radical of one of the above compounds, which is attached to L² through a modifiable oxygen, nitrogen, or carbon atom.

### Moiety for FGFR3

In certain embodiments, the TPL is a moiety that binds to and/or inhibits Fibroblast Growth Factor Receptor 3 (FGFR3). Compounds that bind and/or inhibit FGFR3 are reported in the literature, which include:

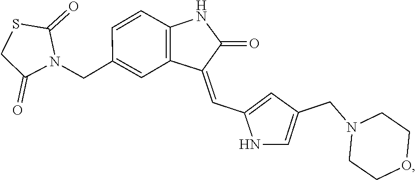 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

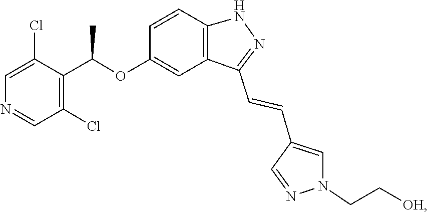

as described in Chen, D. et al., WO 2010/129509;

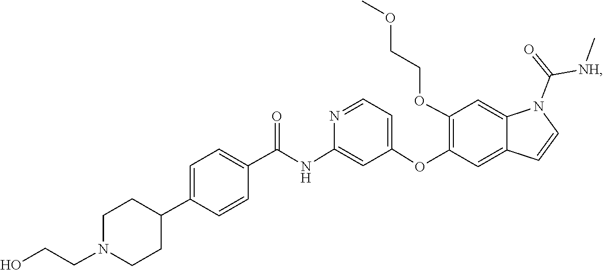 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477

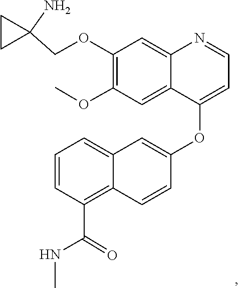 [SMILES: CNC(=O)c1cccc2cc(Oc3ccnc4cc(OCC5(N)CC5)c(OC)cc34)ccc12]

as described in Holmstroem, T. H., et al., Mol Cancer Ther 2019, vol. 18(1), page 28;

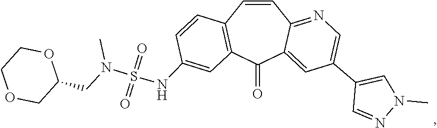 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

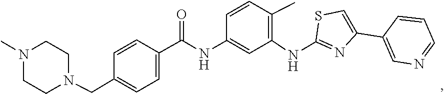 [SMILES: Cc1ccc(NC(=O)c2ccc(CN3CCN(C)CC3)cc2)cc1Nc1nc(-c2cccnc2)cs1]

as described in Moussy, A. et al., WO 2015/082496;

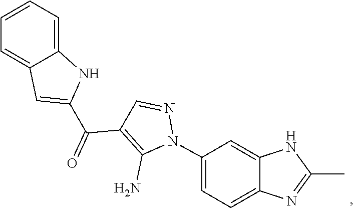 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

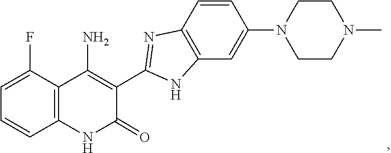 [SMILES: CN1CCN(c2ccc3nc(-c4c(N)c5c(F)cccc5[nH]c4=O)[nH]c3c2)CC1]

as described in Renhowe, P. A. et al., J Med Chem 2009, vol. 52(2), page 278;

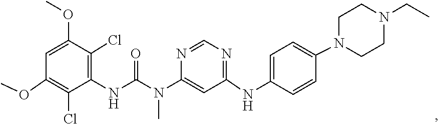 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

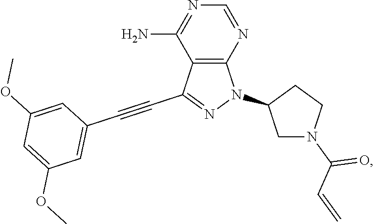 [SMILES: C=CC(=O)N1CC[C@H](n2nc(C#Cc3cc(OC)cc(OC)c3)c3c(N)ncnc32)C1]

as described in Sagara, T. et al., WO 2013/108809;

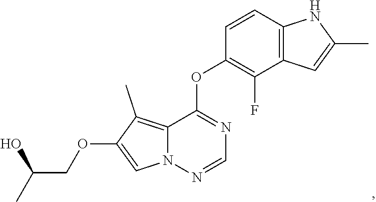 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

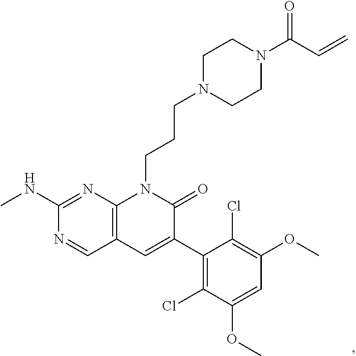 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

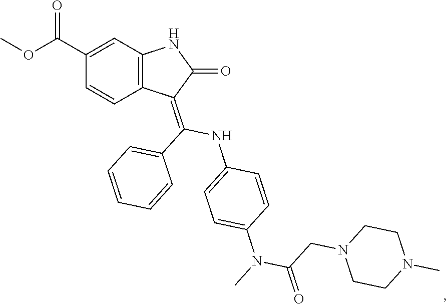

as described in Walters, I. et al., WO 2017/109513;

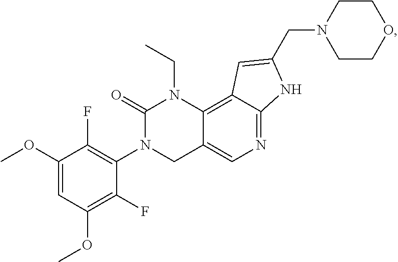 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951.

In certain embodiments, the TPL is a radical of one of the above compounds, which is attached to L² through a modifiable oxygen, nitrogen, or carbon atom.

### Moiety for FGFR4

In certain embodiments, the TPL is a moiety that binds to and/or inhibits Fibroblast Growth Factor Receptor 4 (FGFR4). Compounds that bind and/or inhibit FGFR4 are reported in the literature, which include:

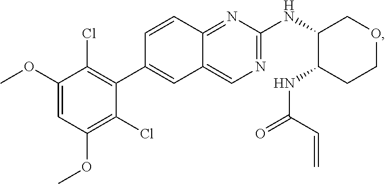 [SMILES: C=CC(=O)N[C@H]1CCOC[C@H]1Nc1ncc2cc(-c3c(Cl)c(OC)cc(OC)c3Cl)ccc2n1]

as described in Bifulco, N. Jr. et al., US2017/174652;

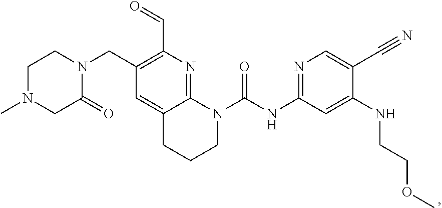

as described in Buschmann, N. et al., WO 2015/059668;

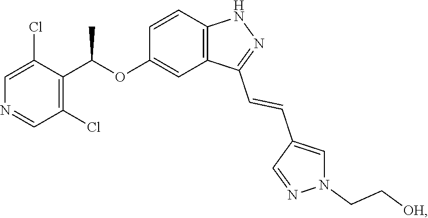

as described in Chen, D. et al., WO 2010/129509;

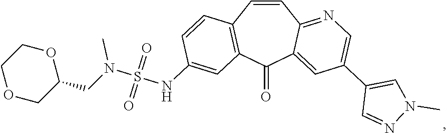 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

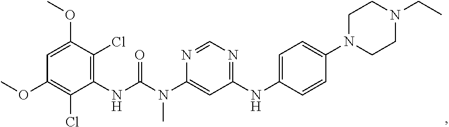 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

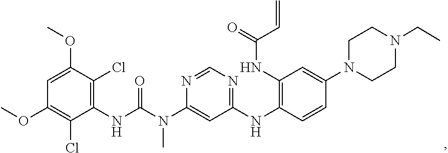 [SMILES: C=CC(=O)Nc1cc(N2CCN(CC)CC2)ccc1Nc1cc(N(C)C(=O)Nc2c(Cl)c(OC)cc(OC)c2Cl)ncn1]

as described in Reynolds, D. et al., WO 2015/057938;

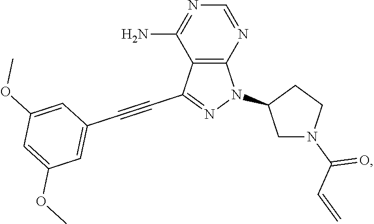 [SMILES: C=CC(=O)N1CC[C@H](n2nc(C#Cc3cc(OC)cc(OC)c3)c3c(N)ncnc32)C1]

as described in Sagara, T. et al., WO 2013/108809;

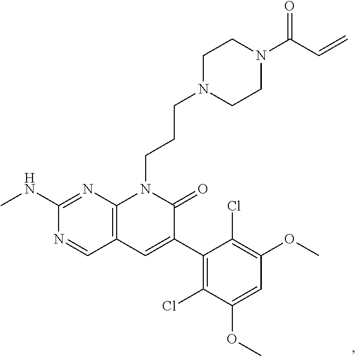 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

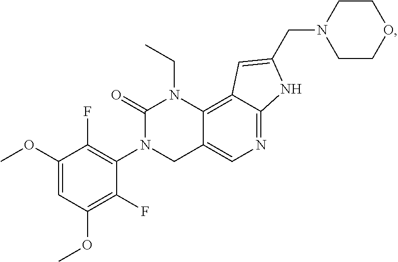 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

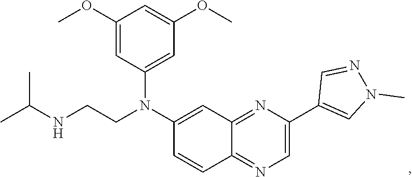

as described in Xu, X. et al., WO 2018/153373.

In certain embodiments, the TPL is a radical of one of the above compounds, which is attached to L² through a modifiable oxygen, nitrogen, or carbon atom.

### Moiety for ERK-1

In certain embodiments, the TPL is a moiety that inhibit extracellular signal-regulated kinase 1 (ERK-1). Compounds that inhibit ERK-1 are reported in the literature, which include:

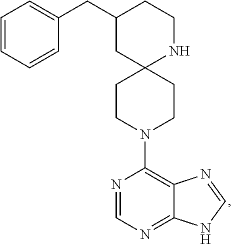 [SMILES: c1ccc(CC2CCNC3(CCN(c4ncnc5[nH]cnc45)CC3)C2)cc1]

as described in Allen, C. E. et al., in Bioorg Med Chem 2013, vol 21(18), page 5707;

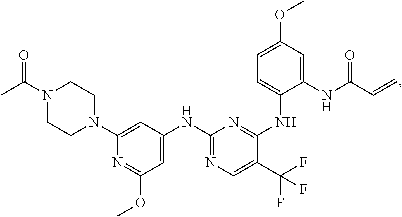 [SMILES: C=CC(=O)Nc1cc(OC)ccc1Nc1nc(Nc2cc(OC)nc(N3CCN(C(C)=O)CC3)c2)ncc1C(F)(F)F]

as described in Haq, N. et al., in WO 2014/124230;

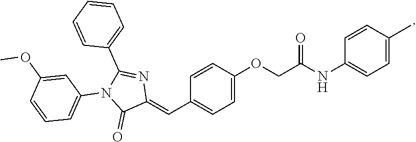

as described in Awadallah, F. M. et al., in Eur J Med Chem 2015, vol 94, page 397;

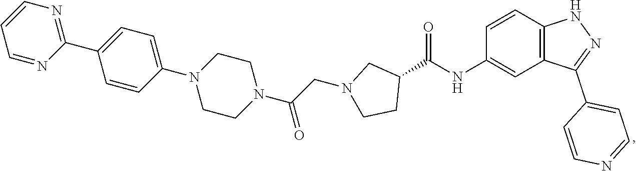 [SMILES: O=C(Nc1ccc2[nH]nc(-c3ccncc3)c2c1)[C@@H]1CCN(CC(=O)N2CCN(c3ccc(-c4ncccn4)cc3)CC2)C1]

as described in Huang, P. Q. et al., in WO 2016/161160;

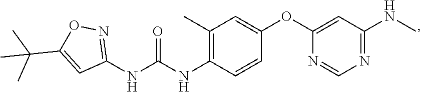 [SMILES: CNc1cc(Oc2ccc(NC(=O)Nc3cc(C(C)(C)C)on3)c(C)c2)ncn1]

as described in Chen, Y. et al., in Eur J Med Chem 2017, vol 127, page 997;

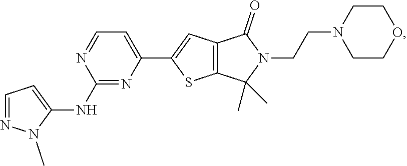 [SMILES: Cn1nccc1Nc1nccc(-c2cc3c(s2)C(C)(C)N(CCN2CCOCC2)C3=O)n1]

as described in Cortez, G. S. et al., in WO 2016/106029;

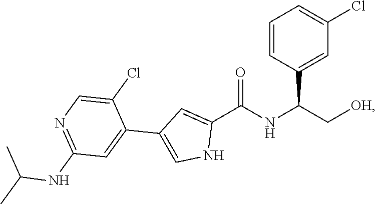 [SMILES: CC(C)Nc1cc(-c2c[nH]c(C(=O)N[C@H](CO)c3cccc(Cl)c3)c2)c(Cl)cn1]

as described in Huang, P. Q. et al., in WO 2016/161160;

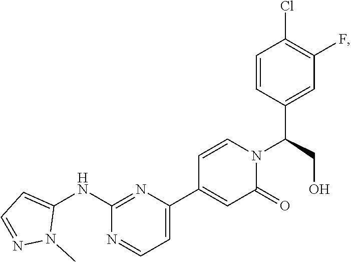 [SMILES: Cn1nccc1Nc1nccc(-c2ccn([C@H](CO)c3ccc(Cl)c(F)c3)c(=O)c2)n1]

as described in Huang, P. Q. et al., in WO 2016/161160;

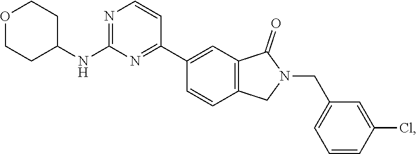 [SMILES: O=C1c2cc(-c3ccnc(NC4CCOCC4)n3)ccc2CN1Cc1cccc(Cl)c1]

as described in Ji, D. Z. et al., in Eur J Med Chem 2019, vol 164, page 334;

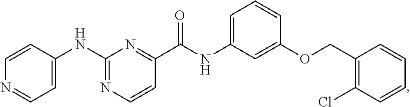 [SMILES: O=C(Nc1cccc(OCc2ccccc2Cl)c1)c1ccnc(Nc2ccncc2)n1]

as described in Kim, E. E. K. et al., in KR2012/092768;

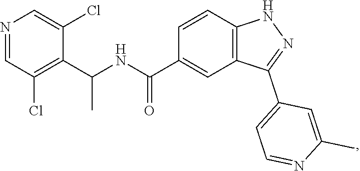 [SMILES: Cc1cc(-c2n[nH]c3ccc(C(=O)NC(C)c4c(Cl)cncc4Cl)cc23)ccn1]

as described in Li, L. et al., in Bioorg Med Chem Lett 2016, vol 26(11), page 2600;

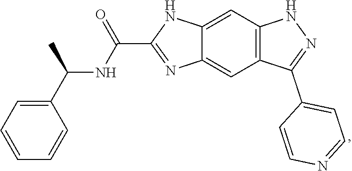 [SMILES: C[C@@H](NC(=O)c1nc2cc3c(-c4ccncc4)n[nH]c3cc2[nH]1)c1ccccc1]

as described in Liu, S. et al., in WO 2019/076336;

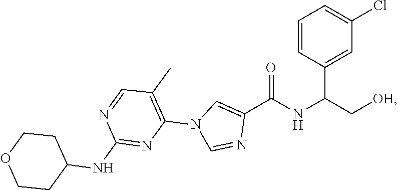 [SMILES: Cc1cnc(NC2CCOCC2)nc1-n1cnc(C(=O)NC(CO)c2cccc(Cl)c2)c1]

as described in Venkatesan, A. M. et al., in U.S. Pat. No. 9,896,445;

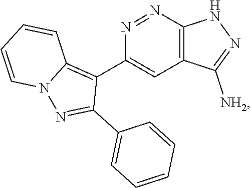 [SMILES: Nc1n[nH]c2nnc(-c3c(-c4ccccc4)nn4ccccc34)cc12]

as described in Zhang, C. et al., in J Pharmacol Exp Ther 2019, vol 370(2), page 206.

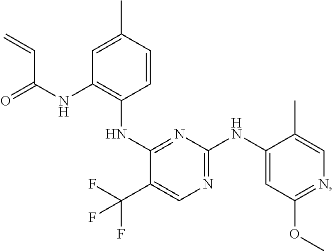 [SMILES: C=CC(=O)Nc1cc(C)ccc1Nc1nc(Nc2cc(OC)ncc2C)ncc1C(F)(F)F]

as described in Haq, N. et al., in WO 2014/124230.

In certain embodiments, the TPL is a radical of one of the above compounds, which is attached to L² through a modifiable oxygen, nitrogen, or carbon atom.

### Moiety for ERK-2

In certain embodiments, the TPL is a moiety that inhibits extracellular signal-regulated kinase 2 (ERK-2). Compounds that inhibit ERK-2 are reported in the literature, which include:

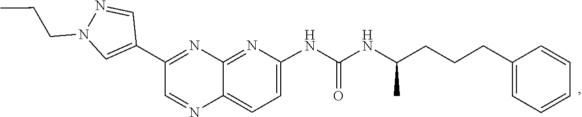 [SMILES: CCCn1cc(-c2cnc3ccc(NC(=O)N[C@H](C)CCCc4ccccc4)nc3n2)cn1]

as described in Gerlach, M. et al., in WO 2012/136691;

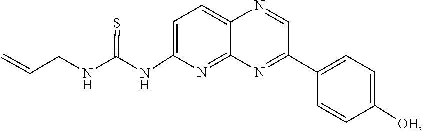 [SMILES: C=CCNC(=S)Nc1ccc2ncc(-c3ccc(O)cc3)nc2n1]

as described in Guenther, E. et al., in WO 2004/104002;

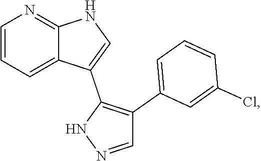 [SMILES: Clc1cccc(-c2cn[nH]c2-c2c[nH]c3ncccc23)c1]

as described in Fairfax, D. et al., in WO 2012/094313;

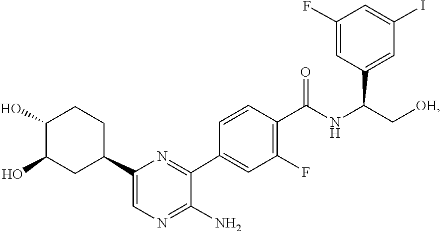 [SMILES: Nc1ncc([C@@H]2CC[C@@H](O)[C@H](O)C2)nc1-c1ccc(C(=O)N[C@H](CO)c2cc(F)cc(I)c2)c(F)c1]

as described in Bagdanoff, J. T. et al., in WO 2015/066188;

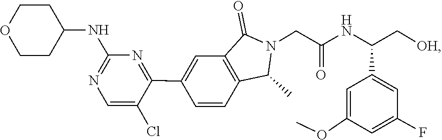 [SMILES: COc1cc(F)cc([C@@H](CO)NC(=O)CN2C(=O)c3cc(-c4nc(NC5CCOCC5)ncc4Cl)ccc3[C@H]2C)c1]

as described in Berdini, V. et al., in WO 2017/068412;

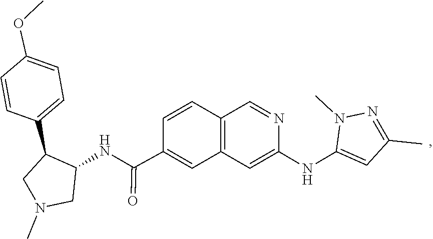 [SMILES: COc1ccc([C@@H]2CN(C)C[C@H]2NC(=O)c2ccc3cnc(Nc4cc(C)nn4C)cc3c2)cc1]

as described in Blake, J. et al., in WO 2014/036015;

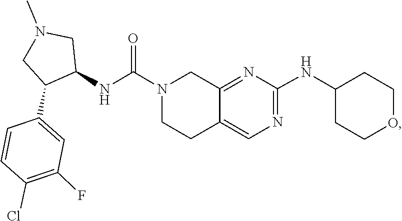 [SMILES: CN1C[C@@H](NC(=O)N2CCc3cnc(NC4CCOCC4)nc3C2)[C@H](c2ccc(Cl)c(F)c2)C1]

as described in Blake, J. F. et al., in WO 2012/118850;

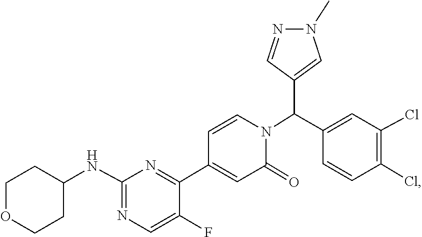 [SMILES: Cn1cc(C(c2ccc(Cl)c(Cl)c2)n2ccc(-c3nc(NC4CCOCC4)ncc3F)cc2=O)cn1]

as described in Blake, J. F. et al., in WO 2013/130976;

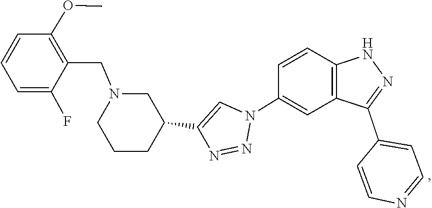 [SMILES: COc1cccc(F)c1CN1CCC[C@@H](c2cn(-c3ccc4[nH]nc(-c5ccncc5)c4c3)nn2)C1]

as described in Boga, S. B. et al., in WO 2012/058127;

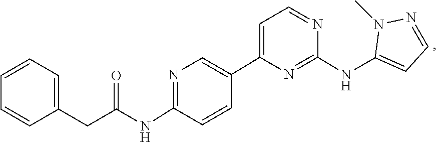 [SMILES: Cn1nccc1Nc1nccc(-c2ccc(NC(=O)Cc3ccccc3)nc2)n1]

as described in Cao, J. et al., in WO 2017/114510;

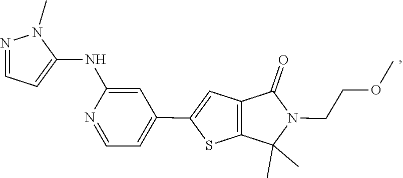 [SMILES: COCCN1C(=O)c2cc(-c3ccnc(Nc4ccnn4C)c3)sc2C1(C)C]

as described in Cortez, G. S. et al., in WO 2016/106009;

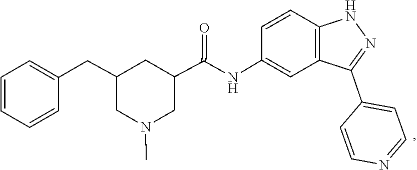 [SMILES: CN1CC(Cc2ccccc2)CC(C(=O)Nc2ccc3[nH]nc(-c4ccncc4)c3c2)C1]

as described in Deng, Y. et al., in WO 2012/030685;

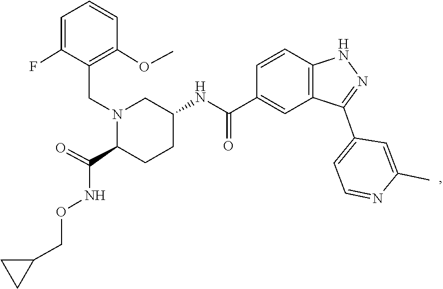 [SMILES: COc1cccc(F)c1CN1C[C@H](NC(=O)c2ccc3[nH]nc(-c4ccnc(C)c4)c3c2)CC[C@H]1C(=O)NOCC1CC1]

as described in Deng, Y. et al., in WO 2011/163330;

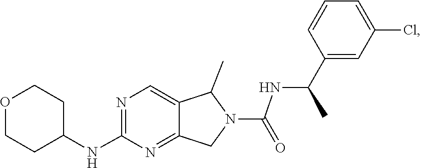 [SMILES: CC1c2cnc(NC3CCOCC3)nc2CN1C(=O)N[C@H](C)c1cccc(Cl)c1]

as described in Dillon, M. P. et al., in WO 2014/047020;

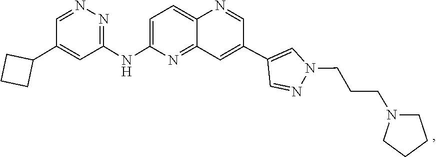 [SMILES: c1nc2ccc(Nc3cc(C4CCC4)cnn3)nc2cc1-c1cnn(CCCN2CCCC2)c1]

as described in Furuyama, H. et al., in WO 2014/109414;

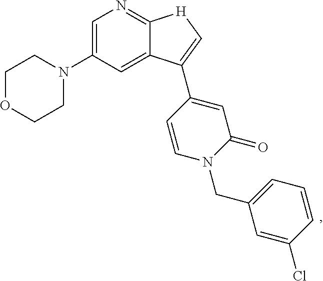

as described in Guichou, J.-F. et al., in WO 2017/085230;

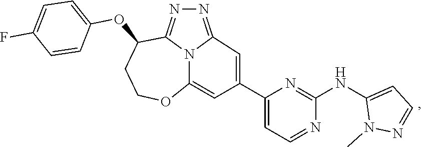 [SMILES: Cn1nccc1Nc1nccc(-c2cc3n4c(nnc4c2)[C@H](Oc2ccc(F)cc2)CCO3)n1]

as described in Kolesnikov, A. et al., in WO 2015/085007;

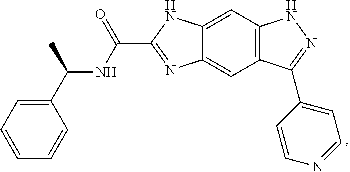 [SMILES: C[C@@H](NC(=O)c1nc2cc3c(-c4ccncc4)n[nH]c3cc2[nH]1)c1ccccc1]

as described in Liu, S. et al., in WO 2019/076336;

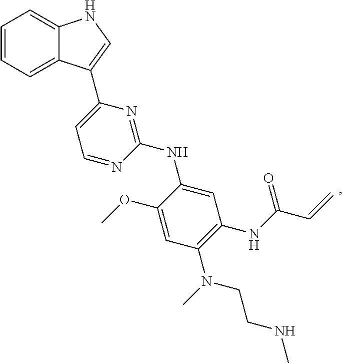 [SMILES: C=CC(=O)Nc1cc(Nc2nccc(-c3c[nH]c4ccccc34)n2)c(OC)cc1N(C)CCNC]

as described in Tang, J. et al., in CN107973783;

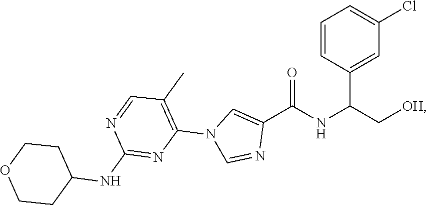 [SMILES: Cc1cnc(NC2CCOCC2)nc1-n1cnc(C(=O)NC(CO)c2cccc(Cl)c2)c1]

as described in Venkatesan, A. M. et al., in US 2016/362406;

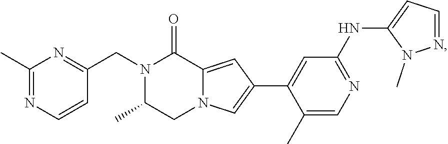 [SMILES: Cc1nccc(CN2C(=O)c3cc(-c4cc(Nc5ccnn5C)ncc4C)cn3C[C@@H]2C)n1]

as described in Ward, R. A. et al., in WO 2017/080980;

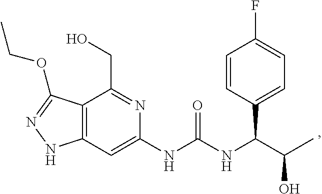 [SMILES: CCOc1n[nH]c2cc(NC(=O)N[C@@H](c3ccc(F)cc3)[C@@H](C)O)nc(CO)c12]

as described in Wilson, K. J. et al., in WO 2014/052566;

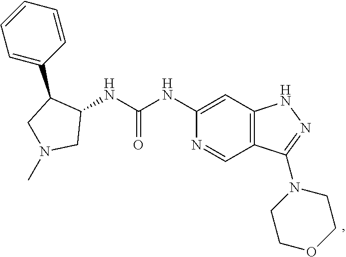 [SMILES: CN1C[C@@H](NC(=O)Nc2cc3[nH]nc(N4CCOCC4)c3cn2)[C@H](c2ccccc2)C1]

as described in Wilson, K. J. et al., in WO 2014/052563;

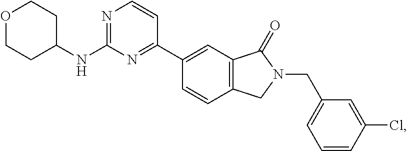 [SMILES: O=C1c2cc(-c3ccnc(NC4CCOCC4)n3)ccc2CN1Cc1cccc(Cl)c1]

as described in Xu, Y. et al., in CN109608444.

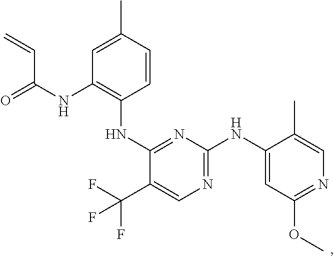 [SMILES: C=CC(=O)Nc1cc(C)ccc1Nc1nc(Nc2cc(OC)ncc2C)ncc1C(F)(F)F]

as described in Haq, N. et al., in WO2014/124230.

In certain embodiments, the TPL is a radical of one of the above compounds, which is attached to L² through a modifiable oxygen, nitrogen, or carbon atom.

### Moiety for FGR

In certain embodiments, the TPL is a moiety that inhibits tyrosine-protein kinase FGR (FGR). Compounds that inhibit FGR are reported in the literature, which include:

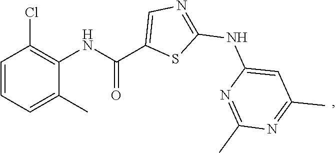 [SMILES: Cc1cc(Nc2ncc(C(=O)Nc3c(C)cccc3Cl)s2)nc(C)n1]

as described in Chen, P. et al., in Bioorg Med Chem Lett 2004, 14(24): 6061.

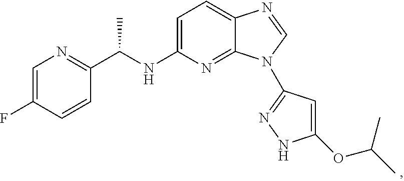 [SMILES: CC(C)Oc1cc(-n2cnc3ccc(N[C@@H](C)c4ccc(F)cn4)nc32)n[nH]1]

as described in Wang, T. et al., in ACS Med Chem Lett 2012, 3(9): 705.

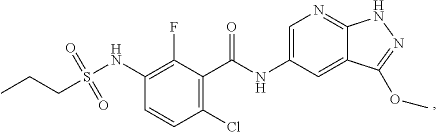 [SMILES: CCCS(=O)(=O)Nc1ccc(Cl)c(C(=O)Nc2cnc3[nH]nc(OC)c3c2)c1F]

as described in Wenglowsky, S. et al., in ACS Med Chem Lett 2011, 2(5): 342.

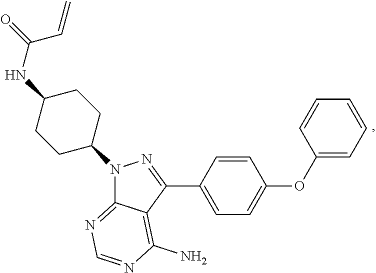 [SMILES: C=CC(=O)N[C@H]1CC[C@@H](n2nc(-c3ccc(Oc4ccccc4)cc3)c3c(N)ncnc32)CC1]

as described in Buggy, J. et al., in US 2012/184567.

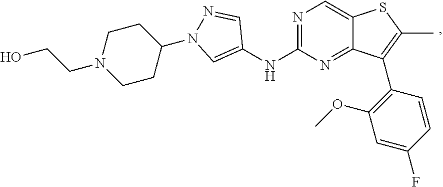 [SMILES: COc1cc(F)ccc1-c1c(C)sc2cnc(Nc3cnn(C4CCN(CCO)CC4)c3)nc12]

as described in Wang, T. et al., in WO 2017/012559.

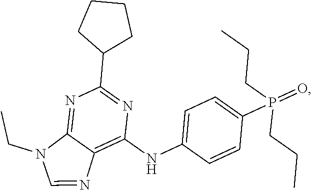 [SMILES: CCCP(=O)(CCC)c1ccc(Nc2nc(C3CCCC3)nc3c2ncn3CC)cc1]

as described in Summy, J. et al., in Mol Cancer Ther 2005, 4(12): 1900.

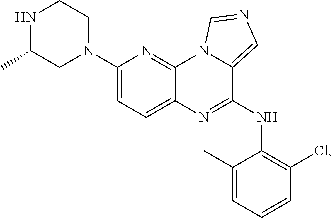 [SMILES: Cc1cccc(Cl)c1Nc1nc2ccc(N3CCN[C@@H](C)C3)nc2n2cncc12]

as described in Chen, P. et al., in J Med Chem 2004, 47(18): 4517.

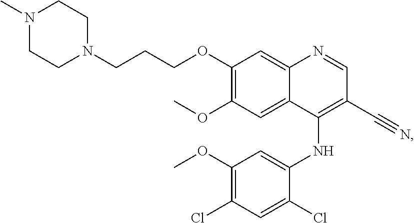 [SMILES: COc1cc(Nc2c(C#N)cnc3cc(OCCCN4CCN(C)CC4)c(OC)cc23)c(Cl)cc1Cl]

as described in Puttini, M. et al., in Cancer Res 2006, 66(23): 11314.

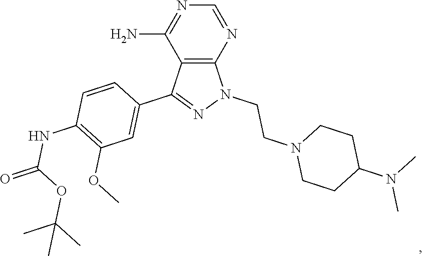 [SMILES: COc1cc(-c2nn(CCN3CCC(N(C)C)CC3)c3ncnc(N)c23)ccc1NC(=O)OC(C)(C)C]

as described in Fraser, C. et al., in J Med Chem 2016, 59(10): 4697.

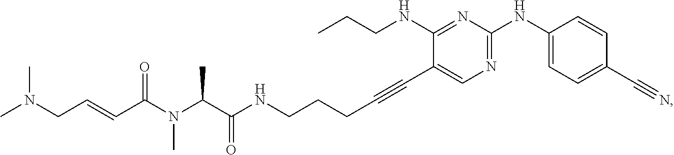 [SMILES: CCCNc1nc(Nc2ccc(C#N)cc2)ncc1C#CCCCNC(=O)[C@H](C)N(C)C(=O)/C=C/CN(C)C]

as described in Yamaura, T. et al., in Blood 2018, 131(4): 426.

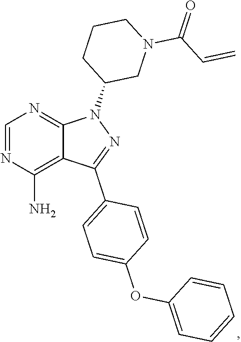 [SMILES: C=CC(=O)N1CCC[C@@H](n2nc(-c3ccc(Oc4ccccc4)cc3)c3c(N)ncnc32)C1]

as described in Wu, H. et al., in ACS Chem Biol 2014, 9(5): 1086.

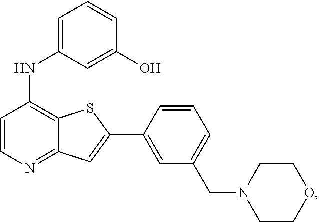 [SMILES: Oc1cccc(Nc2ccnc3cc(-c4cccc(CN5CCOCC5)c4)sc23)c1]

as described in Sun, X. et al., in J Pharmacol Exp Ther 2012, 340(3): 510.

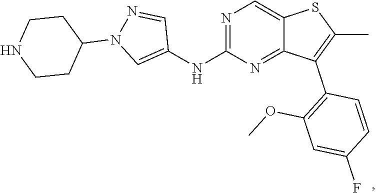 [SMILES: COc1cc(F)ccc1-c1c(C)sc2cnc(Nc3cnn(C4CCNCC4)c3)nc12]

as described in Wang, T. et al., in WO 2017/012559.

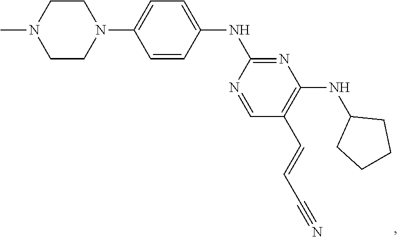 [SMILES: CN1CCN(c2ccc(Nc3ncc(/C=C/C#N)c(NC4CCCC4)n3)cc2)CC1]

as described in Reddy, M. et al., in 255th Am Chem Soc (ACS) Natl Meet⋅Mar. 18, 20188/Mar. 22, 2018⋅New Orleans, United States⋅Abst MEDI 34.

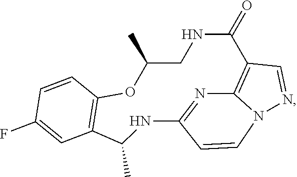 [SMILES: C[C@H]1CNC(=O)c2cnn3ccc(nc23)N[C@H](C)c2cc(F)ccc2O1]

as described in Drilon, A. et al., in Cancer Discov 2018, 8(10): 1227.

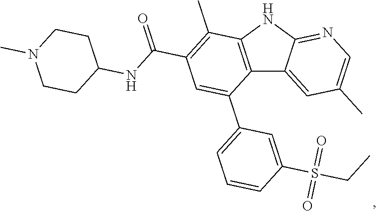 [SMILES: CCS(=O)(=O)c1cccc(-c2cc(C(=O)NC3CCN(C)CC3)c(C)c3[nH]c4ncc(C)cc4c23)c1]

as described in Farrell, P. et al., in Mol Cancer Ther 2013, 12(4): 460.

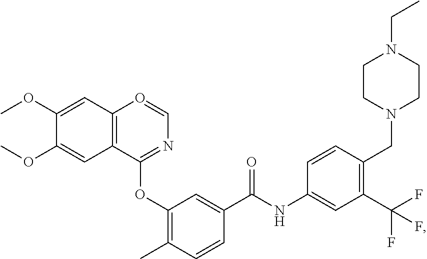

as described in Weir, M. et al., in ACS Chem Biol 2018, 13(6): 1551.

In certain embodiments, the TPL is a radical of one of the above compounds, which is attached to L² through a modifiable oxygen, nitrogen, or carbon atom.

### Moiety for HER3

In certain embodiments, the TPL is a moiety that inhibits receptor tyrosine-protein kinase erbB-3 (HER3). Compounds that inhibit HER3 are reported in the literature, which include:

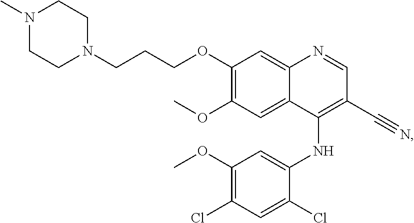 [SMILES: COc1cc(Nc2c(C#N)cnc3cc(OCCCN4CCN(C)CC4)c(OC)cc23)c(Cl)cc1Cl]

as described in Li, L. et al., in Leuk Res 2019, 78: 12.

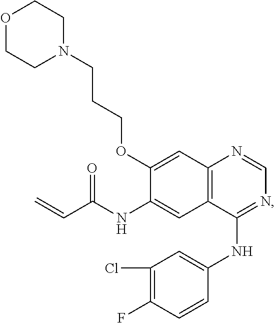 [SMILES: C=CC(=O)Nc1cc2c(Nc3ccc(F)c(Cl)c3)ncnc2cc1OCCCN1CCOCC1]

as described in Allen, L. et al., in Semin Oncol 2002, 29(3, Suppl. 11): 11.

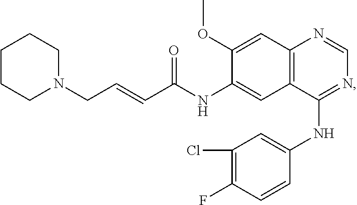 [SMILES: COc1cc2ncnc(Nc3ccc(F)c(Cl)c3)c2cc1NC(=O)/C=C/CN1CCCCC1]

as described in Tecle, H. et al., in US2013274275.

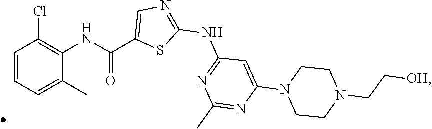 [SMILES: Cc1nc(Nc2ncc(C(=O)Nc3c(C)cccc3Cl)s2)cc(N2CCN(CCO)CC2)n1]

as described in Li, L. et al., in Leuk Res 2019, 78: 12.

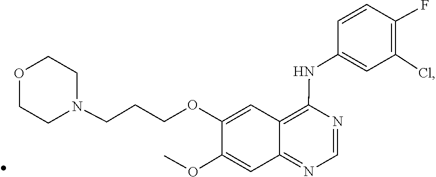 [SMILES: COc1cc2ncnc(Nc3ccc(F)c(Cl)c3)c2cc1OCCCN1CCOCC1]

as described in Marshall, G. et al., in 32nd Annu San Antonio Breast Cancer Symp⋅Dec. 10, 2009/Dec. 13, 2009⋅San Antonio, United States⋅Abst 5059, Cancer Res 2009, 69 (24, Suppl. 3).

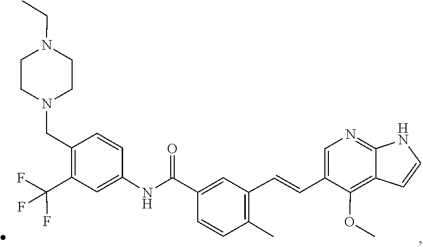 [SMILES: CCN1CCN(Cc2ccc(NC(=O)c3ccc(C)c(/C=C/c4cnc5[nH]ccc5c4OC)c3)cc2C(F)(F)F)CC1]

as described in Tan, L. et al., in J Med Chem 2015, 58(1): 183.

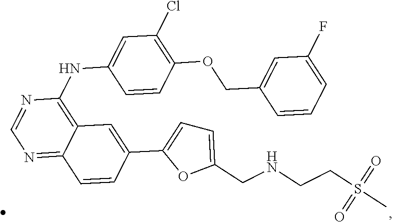 [SMILES: CS(=O)(=O)CCNCc1ccc(-c2ccc3ncnc(Nc4ccc(OCc5cccc(F)c5)c(Cl)c4)c3c2)o1]

as described in Marshall, G. et al., in 32nd Annu San Antonio Breast Cancer Symp⋅Dec. 10, 2009/Dec. 13, 2009⋅San Antonio, United States⋅Abst 5059, Cancer Res 2009, 69 (24, Suppl. 3).

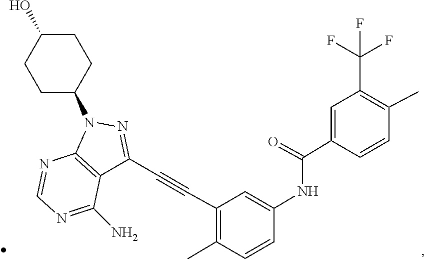 [SMILES: Cc1ccc(NC(=O)c2ccc(C)c(C(F)(F)F)c2)cc1C#Cc1nn([C@H]2CC[C@H](O)CC2)c2ncnc(N)c12]

as described in Zhang, C. et al., in J Med Chem 2016, 59(21): 9788.

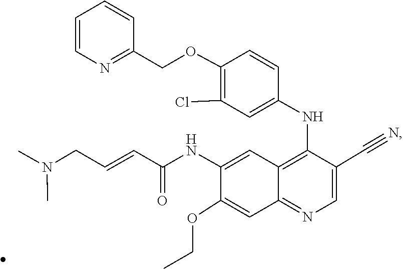 [SMILES: CCOc1cc2ncc(C#N)c(Nc3ccc(OCc4ccccn4)c(Cl)c3)c2cc1NC(=O)/C=C/CN(C)C]

as described in Li, L. et al., in Leuk Res 2019, 78: 12.

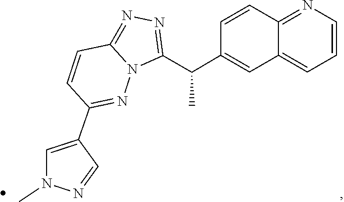 [SMILES: C[C@@H](c1ccc2ncccc2c1)c1nnc2ccc(-c3cnn(C)c3)nn12]

as described in Christensen, G. et al., in Proc Am Assoc Cancer Res (AACR) 2008, 49, Abst.

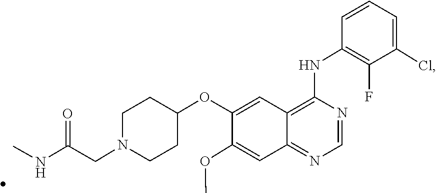 [SMILES: CNC(=O)CN1CCC(Oc2cc3c(Nc4cccc(Cl)c4F)ncnc3cc2OC)CC1]

as described in Dong, X. et al., in Neoplasia 2016, 18(3): 162.

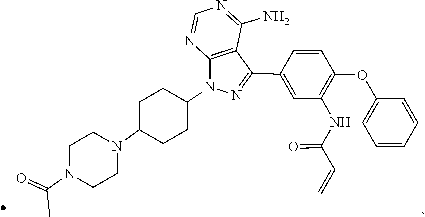 [SMILES: C=CC(=O)Nc1cc(-c2nn(C3CCC(N4CCN(C(C)=O)CC4)CC3)c3ncnc(N)c23)ccc1Oc1ccccc1]

as described in Lim, S. et al., in Bioorg Med Chem Lett 2015, 25(16): 3382.

In certain embodiments, the TPL is a radical of one of the above compounds, which is attached to L² through a modifiable oxygen, nitrogen, or carbon atom.

### Moiety for HER4

In certain embodiments, the TPL is a moiety that inhibits receptor tyrosine-protein kinase erbB-4 (HER4). Compounds that inhibit HER4 are reported in the literature, which include:

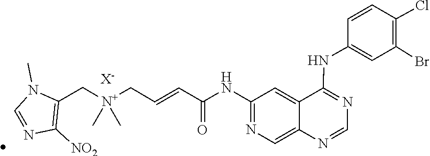

where X is an anion (e.g., Cl⁻), as described in Smaill, J. et al., in WO 2011/028135.

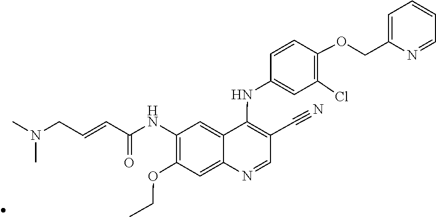 [SMILES: CCOc1cc2ncc(C#N)c(Nc3ccc(OCc4ccccn4)c(Cl)c3)c2cc1NC(=O)/C=C/CN(C)C]

as described in Wissner, A. et al., in WO 2005/028443.

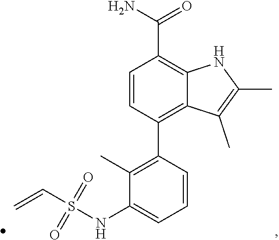 [SMILES: C=CS(=O)(=O)Nc1cccc(-c2ccc(C(N)=O)c3[nH]c(C)c(C)c23)c1C]

as described in Liu, Q. et al., in Bioorg Med Chem Lett 2018, 28(18): 3080.

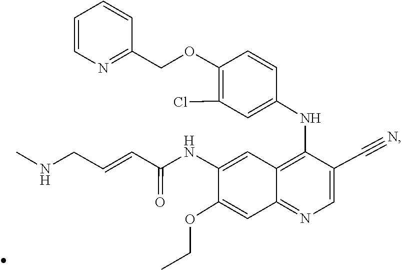 [SMILES: CCOc1cc2ncc(C#N)c(Nc3ccc(OCc4ccccn4)c(Cl)c3)c2cc1NC(=O)/C=C/CNC]

as described in McGinnis, J. et al., in WO 2006/127203.

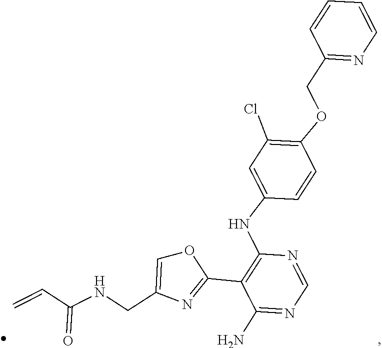 [SMILES: C=CC(=O)NCc1coc(-c2c(N)ncnc2Nc2ccc(OCc3ccccn3)c(Cl)c2)n1]

as described in Cha, M. et al., in J Med Chem 2012, 55(6): 2846.

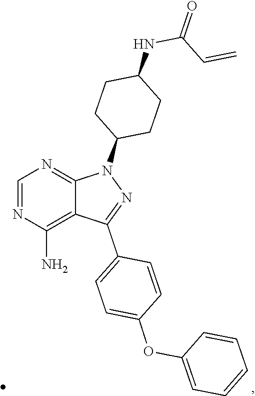 [SMILES: C=CC(=O)N[C@H]1CC[C@@H](n2nc(-c3ccc(Oc4ccccc4)cc3)c3c(N)ncnc32)CC1]

as described in Chen, W. et al., in US 2012/184567.

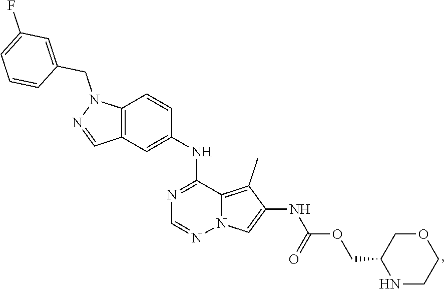 [SMILES: Cc1c(NC(=O)OC[C@@H]2COCCN2)cn2ncnc(Nc3ccc4c(cnn4Cc4cccc(F)c4)c3)c12]

as described in Gavai, A. et al., in J Med Chem 2009, 52(21): 6527.

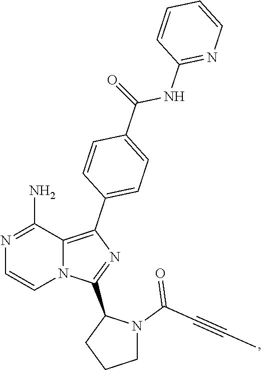 [SMILES: CC#CC(=O)N1CCC[C@H]1c1nc(-c2ccc(C(=O)Nc3ccccn3)cc2)c2c(N)nccn12]

as described in Johnson, D. et al., in WO 2015/110923.

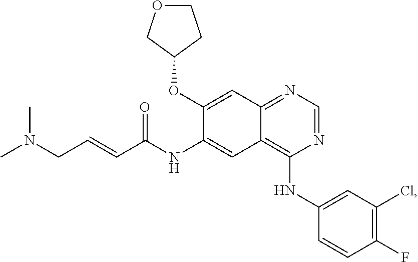 [SMILES: CN(C)C/C=C/C(=O)Nc1cc2c(Nc3ccc(F)c(Cl)c3)ncnc2cc1O[C@H]1CCOC1]

as described in Solca, F. et al., in J Pharmacol Exp Ther 2012, 343(2): 342.

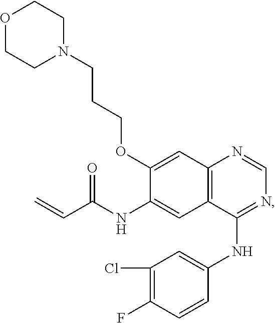 [SMILES: C=CC(=O)Nc1cc2c(Nc3ccc(F)c(Cl)c3)ncnc2cc1OCCCN1CCOCC1]

as described in Solca, F. et al., in J Pharmacol Exp Ther 2012, 343(2): 342.

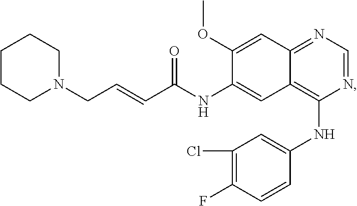 [SMILES: COc1cc2ncnc(Nc3ccc(F)c(Cl)c3)c2cc1NC(=O)/C=C/CN1CCCCC1]

as described in Smaill, J. et al., in J Med Chem 2016, 59(17): 8103.

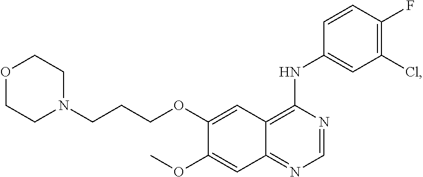 [SMILES: COc1cc2ncnc(Nc3ccc(F)c(Cl)c3)c2cc1OCCCN1CCOCC1]

as described in Wood, E. et al., in Cancer Res 2004, 64(18): 6652.

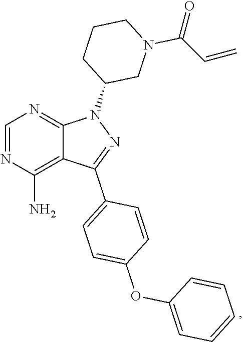 [SMILES: C=CC(=O)N1CCC[C@@H](n2nc(-c3ccc(Oc4ccccc4)cc3)c3c(N)ncnc32)C1]

as described in Verner, E. et al., in US2012184013.

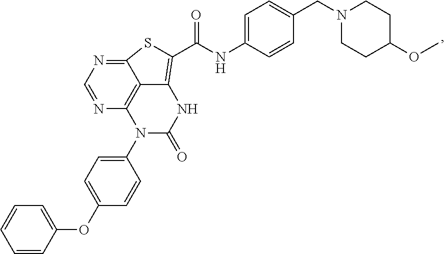 [SMILES: COC1CCN(Cc2ccc(NC(=O)c3sc4ncnc5c4c3NC(=O)N5c3ccc(Oc4ccccc4)cc3)cc2)CC1]

as described in Pandey, N. et al., in Proc Am Assoc Cancer Res (AACR) 2006, 47, Abst 4747.

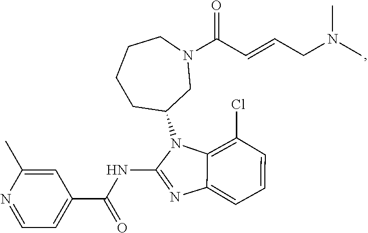

as described in Lelais, G. et al., in J Med Chem 2016, 59(14): 6671.

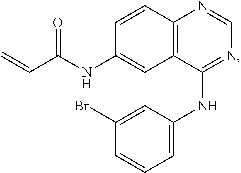 [SMILES: C=CC(=O)Nc1ccc2ncnc(Nc3cccc(Br)c3)c2c1]

as described in Hur, W. et al., in Bioorg Med Chem Lett 2008, 18(22): 5916.

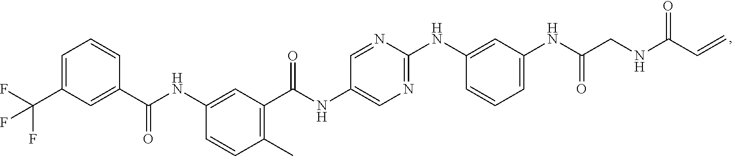 [SMILES: C=CC(=O)NCC(=O)Nc1cccc(Nc2ncc(NC(=O)c3cc(NC(=O)c4cccc(C(F)(F)F)c4)ccc3C)cn2)c1]

as described in Li, X. et al., in J Med Chem 2014, 57(12): 5112.

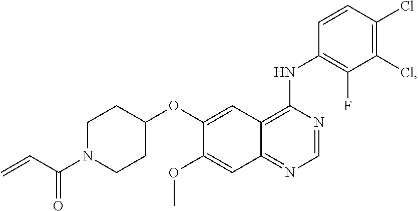 [SMILES: C=CC(=O)N1CCC(Oc2cc3c(Nc4ccc(Cl)c(Cl)c4F)ncnc3cc2OC)CC1]

as described in Cha, M. et al., in Int J Cancer 2012, 130(10): 2445.

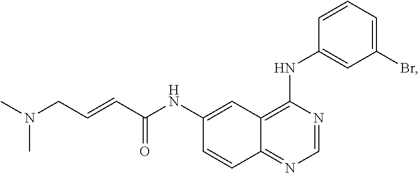 [SMILES: CN(C)C/C=C/C(=O)Nc1ccc2ncnc(Nc3cccc(Br)c3)c2c1]

as described in Smaill, J. et al., in AACR-NCI-EORTC Int Conf Mol Targets Cancer Ther⋅Nov. 15, 2009/Nov. 19, 2009 ⋅Boston, United States⋅Abst C46, Mol Cancer Ther 2009, 8 (12, Suppl. 1).

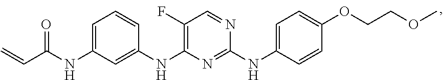 [SMILES: C=CC(=O)Nc1cccc(Nc2nc(Nc3ccc(OCCOC)cc3)ncc2F)c1]

as described in Kaptein, A. et al., in 60th Annu Meet Am Soc Hematol⋅Dec. 1, 2018/Dec. 4, 2018⋅San Diego, United States Abst⋅1871, Blood 2018, 132 (Suppl. 1).

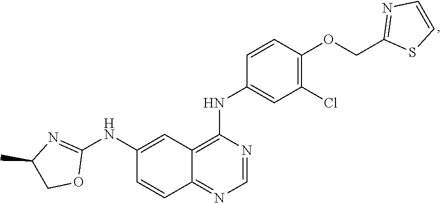 [SMILES: C[C@@H]1COC(Nc2ccc3ncnc(Nc4ccc(OCc5nccs5)c(Cl)c4)c3c2)=N1]

as described in Ooi, A. et al., in 107th Annu Meet Am Assoc Cancer Res (AACR)⋅Apr. 16, 2016/Apr. 20, 2016⋅New Orleans, United States⋅Abst 4719, Cancer Res 2016, 76 (14, Suppl.).

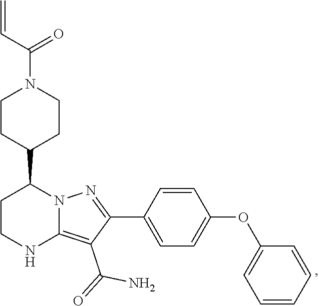 [SMILES: C=CC(=O)N1CCC([C@@H]2CCNc3c(C(N)=O)c(-c4ccc(Oc5ccccc5)cc4)nn32)CC1]

as described in Guo, Y. et al., in J Med Chem 2019, 62(17): 7923.

In certain embodiments, the TPL is a radical of one of the above compounds, which is attached to L² through a modifiable oxygen, nitrogen, or carbon atom.

### Moiety for FLT3

In certain embodiments, the TPL is a moiety inhibits receptor-type tyrosine-protein kinase FLT3 (FLT3). Compounds that inhibit FLT3 are reported in the literature, which include:

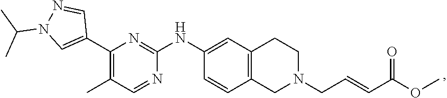 [SMILES: COC(=O)/C=C/CN1CCc2cc(Nc3ncc(C)c(-c4cnn(C(C)C)c4)n3)ccc2C1]

as described in Yang, T. et al., in J Med Chem 2020, 63(23): 14921.

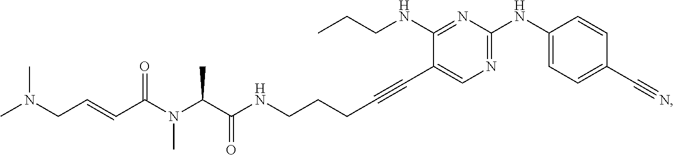 [SMILES: CCCNc1nc(Nc2ccc(C#N)cc2)ncc1C#CCCCNC(=O)[C@H](C)N(C)C(=O)/C=C/CN(C)C]

as described in Mizumoto, S. et al., in WO2015056683.

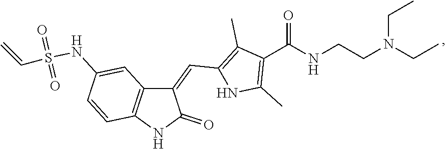 [SMILES: C=CS(=O)(=O)Nc1ccc2c(c1)/C(=C/c1[nH]c(C)c(C(=O)NCCN(CC)CC)c1C)C(=O)N2]

as described in Bensinger, D. et al., in J Med Chem 2019, 62(5): 2428.

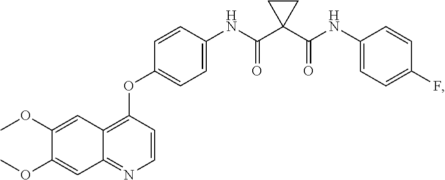 [SMILES: COc1cc2nccc(Oc3ccc(NC(=O)C4(C(=O)Nc5ccc(F)cc5)CC4)cc3)c2cc1OC]

as described in Xu, Q. et al., in Bioorg Med Chem Lett 2019, 29(19), 126630.

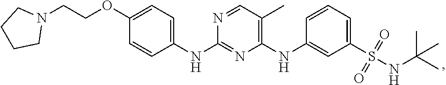 [SMILES: Cc1cnc(Nc2ccc(OCCN3CCCC3)cc2)nc1Nc1cccc(S(=O)(=O)NC(C)(C)C)c1]

as described in Wan, H. et al., in ACS Med Chem Lett 2015, 6(8): 850.

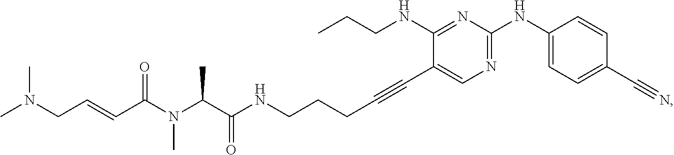 [SMILES: CCCNc1nc(Nc2ccc(C#N)cc2)ncc1C#CCCCNC(=O)[C@H](C)N(C)C(=O)/C=C/CN(C)C]

as described in Nakatani, T. et al., in 57th Annu Meet Am Soc Hematol⋅Dec. 5, 2015/Dec. 8, 2015⋅Orlando, United States⋅Abst 1353, Blood 2015, 126(51).

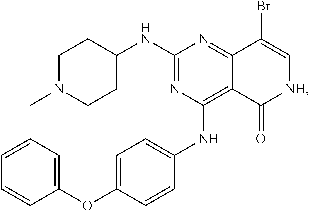 [SMILES: CN1CCC(Nc2nc(Nc3ccc(Oc4ccccc4)cc3)c3c(=O)[nH]cc(Br)c3n2)CC1]

as described in Xin, C. et al., in 110th Annu Meet Am Assoc Cancer Res (AACR)⋅Mar. 29, 2019/Apr. 3, 2019⋅Atlanta, United States⋅Abst 2010, Cancer Res 2019, 79 (13, Suppl.).

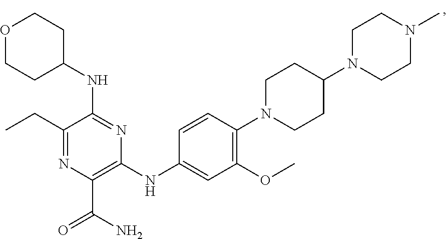 [SMILES: CCc1nc(C(N)=O)c(Nc2ccc(N3CCC(N4CCN(C)CC4)CC3)c(OC)c2)nc1NC1CCOCC1]

as described in Jeong, P. et as., in Eur J Med Chem 2020, 195: 112205.

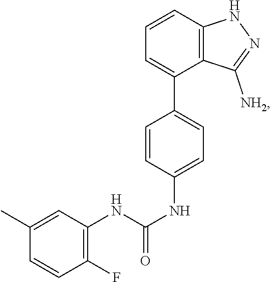 [SMILES: Cc1ccc(F)c(NC(=O)Nc2ccc(-c3cccc4[nH]nc(N)c34)cc2)c1]

as described in Li, J. et al., in Proc Am Assoc Cancer Res (AACR) 2005, 46, Abst 5981.

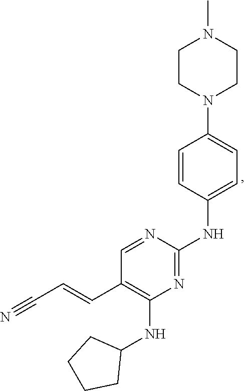 [SMILES: CN1CCN(c2ccc(Nc3ncc(/C=C/C#N)c(NC4CCCC4)n3)cc2)CC1]

as described in Reddy, M. et al., in 255th Am Chem Soc (ACS) Natl Meet⋅Mar. 18, 2018/Mar. 22, 2018⋅New Orleans, United States⋅Abst MEDI 34.

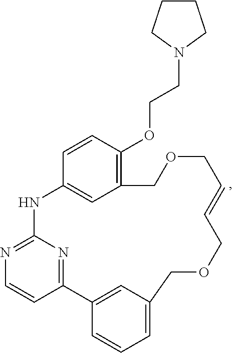 [SMILES: C1=C/COCc2cc(ccc2OCCN2CCCC2)Nc2nccc(n2)-c2cccc(c2)COC/1]

as described in William, A. et al., in J Med Chem 2011, 54(13): 4638.

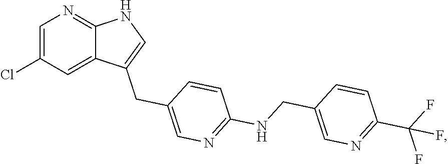 [SMILES: FC(F)(F)c1ccc(CNc2ccc(Cc3c[nH]c4ncc(Cl)cc34)cn2)cn1]

as described in Smith, C. et al., in Cancer Discov 2015, 5(6): 668.

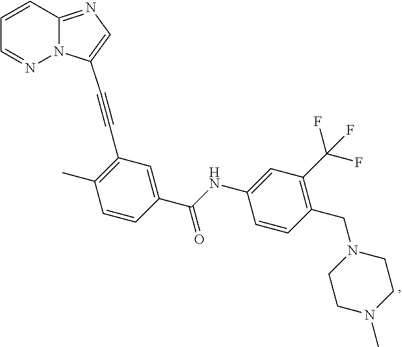 [SMILES: Cc1ccc(C(=O)Nc2ccc(CN3CCN(C)CC3)c(C(F)(F)F)c2)cc1C#Cc1cnc2cccnn12]

as described in Gozgit, J. et al., in Mol Cancer Ther 2011, 10(6): 1028.

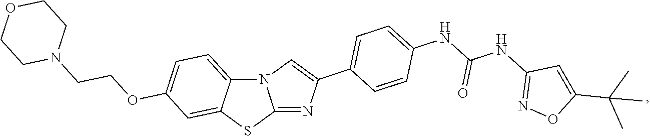 [SMILES: CC(C)(C)c1cc(NC(=O)Nc2ccc(-c3cn4c(n3)sc3cc(OCCN5CCOCC5)ccc34)cc2)no1]

as described in Galanis, A. et al., in 103rd Annu Meet Am Assoc Cancer Res (AACR)⋅Mar. 31, 2012/Apr. 4, 2012⋅Chicago, United States Abst⋅3660, Cancer Res 2012, 72 (Suppl. 8).

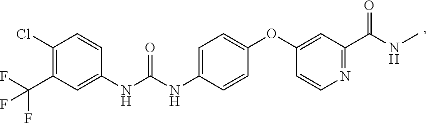 [SMILES: CNC(=O)c1cc(Oc2ccc(NC(=O)Nc3ccc(Cl)c(C(F)(F)F)c3)cc2)ccn1]

as described in Auclair, D. et al., in Proc Am Assoc Cancer Res (AACR) 2005, 46, Abst 5991.

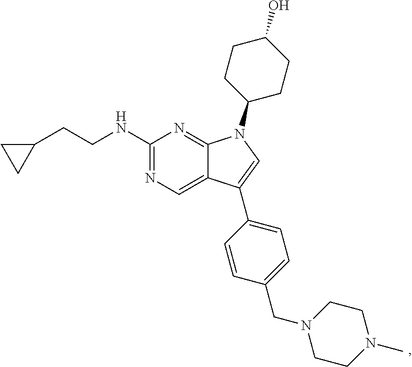 [SMILES: CN1CCN(Cc2ccc(-c3cn([C@H]4CC[C@H](O)CC4)c4nc(NCCC5CC5)ncc34)cc2)CC1]

as described in Minson, K. et al., in 56th Annu Meet Am Soc Hematol⋅Dec. 6, 2014/Dec. 9, 2014⋅San Francisco, United States⋅Abst 3757.

In certain embodiments, the TPL is a radical of one of the above compounds, which is attached to L² through a modifiable oxygen, nitrogen, or carbon atom.

### Moiety for IDH1

In certain embodiments, the TPL is a moiety that binds to and/or inhibits Isocitrate dehydrogenase 1 (IDH1). Compounds that bind and/or inhibit IDH1 are reported in the literature, which include ivosidenib (AG-120), AG-120 (racemic), vorasidenib (AG-881), and BAY 1436032. Additional exemplary compounds that inhibit and/or bind IDH1 are:

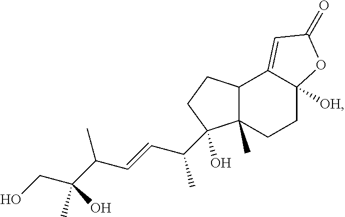 [SMILES: CC(/C=C/[C@@H](C)[C@@]1(O)CCC2C3=CC(=O)O[C@@]3(O)CC[C@@]21C)[C@@](C)(O)CO]

as described by Chen, L. et al., in CN108440471.

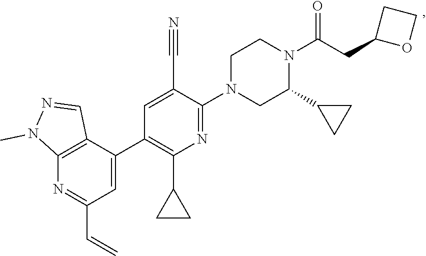 [SMILES: C=Cc1cc(-c2cc(C#N)c(N3CCN(C(=O)C[C@H]4CCO4)[C@H](C4CC4)C3)nc2C2CC2)c2cnn(C)c2n1]

as described by Cao, S. et al., in WO 2012171337.

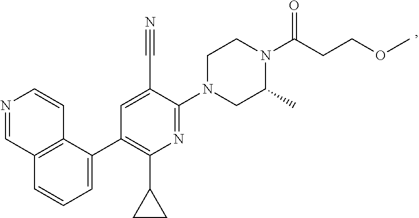 [SMILES: COCCC(=O)N1CCN(c2nc(C3CC3)c(-c3cccc4cnccc34)cc2C#N)C[C@H]1C]

as described by Cao, S. et al., in WO 2012171337.

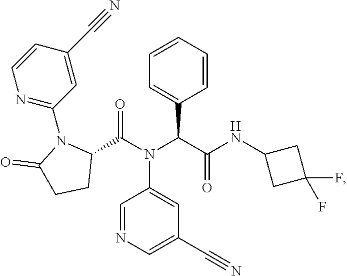 [SMILES: N#Cc1cncc(N(C(=O)[C@@H]2CCC(=O)N2c2cc(C#N)ccn2)[C@H](C(=O)NC2CC(F)(F)C2)c2ccccc2)c1]

as described by Zhou, D. et al., in WO 2015010626.

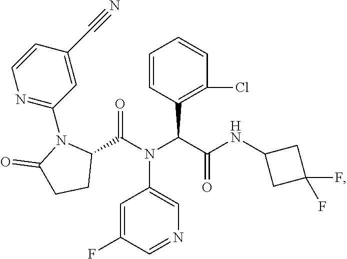 [SMILES: N#Cc1ccnc(N2C(=O)CC[C@H]2C(=O)N(c2cncc(F)c2)[C@H](C(=O)NC2CC(F)(F)C2)c2ccccc2Cl)c1]

as described by Lemieux, R. M. et al., in CN106496090.

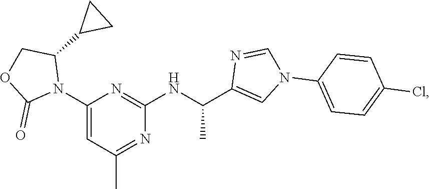 [SMILES: Cc1cc(N2C(=O)OC[C@@H]2C2CC2)nc(N[C@@H](C)c2cn(-c3ccc(Cl)cc3)cn2)n1]

as described by Shultz, M. D. et al., in WO 2014141153.

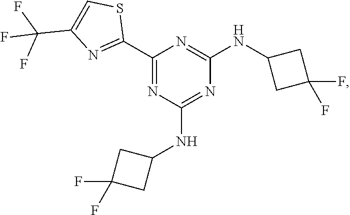 [SMILES: FC1(F)CC(Nc2nc(NC3CC(F)(F)C3)nc(-c3nc(C(F)(F)F)cs3)n2)C1]

as described by Konteatis, Z. D. et al., in WO 2015003640.

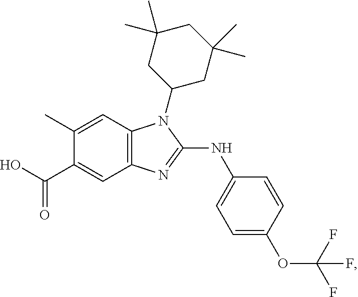 [SMILES: Cc1cc2c(cc1C(=O)O)nc(Nc1ccc(OC(F)(F)F)cc1)n2C1CC(C)(C)CC(C)(C)C1]

as described by Zimmermann, K. et al., in WO 2015121209.

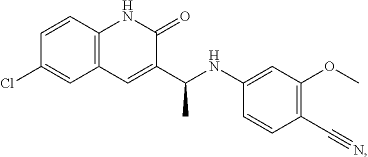 [SMILES: COc1cc(N[C@@H](C)c2cc3cc(Cl)ccc3[nH]c2=O)ccc1C#N]

as described by Lin, J. et al., in US 2018327361.

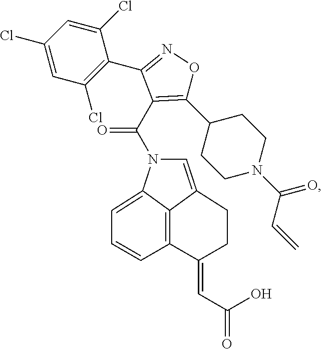 [SMILES: C=CC(=O)N1CCC(c2onc(-c3c(Cl)cc(Cl)cc3Cl)c2C(=O)n2cc3c4c(cccc42)/C(=C/C(=O)O)CC3)CC1]

as described by Matsunaga, H. et al., in WO 2016052697.

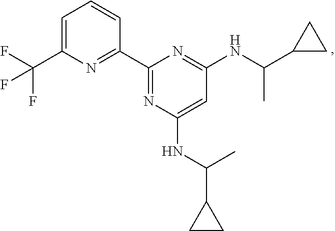 [SMILES: CC(Nc1cc(NC(C)C2CC2)nc(-c2cccc(C(F)(F)F)n2)n1)C1CC1]

as described by Konteatis, Z. D. et al., in US 2016220572.

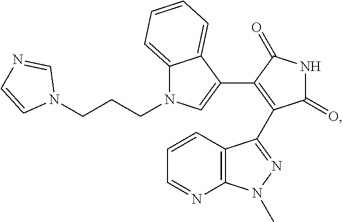 [SMILES: Cn1nc(C2=C(c3cn(CCCn4ccnc4)c4ccccc34)C(=O)NC2=O)c2cccnc21]

as described by Ye, Q. et al., in CN109535158.

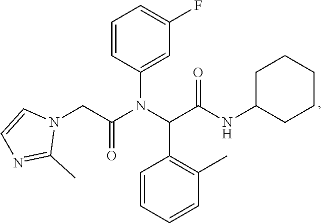 [SMILES: Cc1ccccc1C(C(=O)NC1CCCCC1)N(C(=O)Cn1ccnc1C)c1cccc(F)c1]

as described by Ye, Q. et al., in CN109535158.

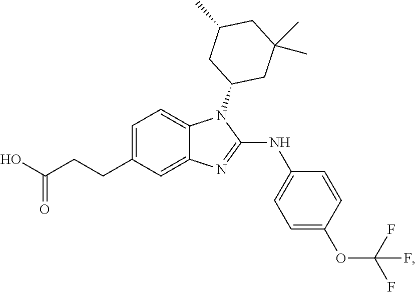 [SMILES: C[C@H]1C[C@@H](n2c(Nc3ccc(OC(F)(F)F)cc3)nc3cc(CCC(=O)O)ccc32)CC(C)(C)C1]

as described by Schirmer, H. et al., in WO 2017016992.

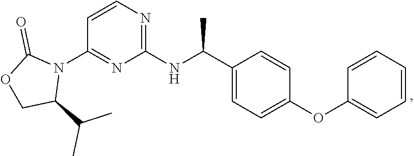 [SMILES: CC(C)[C@H]1COC(=O)N1c1ccnc(N[C@@H](C)c2ccc(Oc3ccccc3)cc2)n1]

as described by Sutton, J. et al., in WO 2013046136.

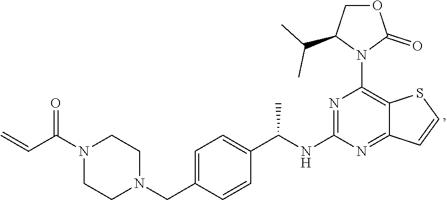 [SMILES: C=CC(=O)N1CCN(Cc2ccc([C@H](C)Nc3nc(N4C(=O)OC[C@@H]4C(C)C)c4sccc4n3)cc2)CC1]

as described by Zhang, T. et al., in WO 2018118793.

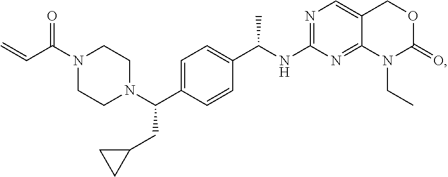 [SMILES: C=CC(=O)N1CCN([C@@H](CC2CC2)c2ccc([C@H](C)Nc3ncc4c(n3)N(CC)C(=O)OC4)cc2)CC1]

as described by Hahn, P. J. et al., in WO 2018111707.

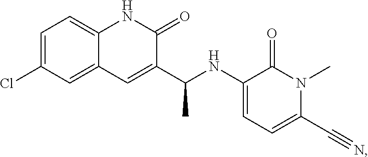 [SMILES: C[C@H](Nc1ccc(C#N)n(C)c1=O)c1cc2cc(Cl)ccc2[nH]c1=O]

as described by Lin, J. et al., in US 2019/0263778.

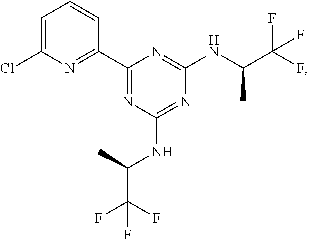 [SMILES: C[C@@H](Nc1nc(N[C@H](C)C(F)(F)F)nc(-c2cccc(Cl)n2)n1)C(F)(F)F]

as described by Cai, Z. et al., in WO 2015003640.

In certain embodiments, the TPL is a radical of one of the above compounds, which is attached to L² through a modifiable oxygen, nitrogen, or carbon atom.

### Additional Features

Compounds of Formula I and/or Formula I-A may be further characterized according to the molecular weight of the TPL. In certain embodiments, the TPL has a molecular weight of less than 1500 Da, 1200 Da, 1000 Da, 800 Da, 600 Da, 400 Da, 300 Da, 200 Da, 150 Da, or 100 Da. Compounds of Formula I may be further characterized according to the molecular weight of the EPL. In certain embodiments, the EPL has a molecular weight of less than 1500 Da, 1200 Da, 1000 Da, 800 Da, 600 Da, 400 Da, 300 Da, 200 Da, 150 Da, or 100 Da.

### Part D: Exemplary Further Description of Linker Component of Compounds of Formula I and I-A

Compounds of Formula I and I-A may be further characterized according to, for example, the identity of the linker component. A variety of linkers are known to one of skill in the art and may be used in the heterobifunctional compounds described herein. For example, in certain embodiments, the linker comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In some embodiments, the linker is symmetrical. In some embodiments, the linker is asymmetric.

In certain embodiments, the linker is a bivalent C₁₋₃₀ saturated or unsaturated, straight or branched, hydrocarbon chain, wherein 1-15 methylene units of L are optionally and independently replaced by cyclopropylene, —N(H)—, —N(C₁₋₄ alkyl)-, —N(C₃₋₅ cycloalkyl)-, —O—, —C(O)—, —OC(O)—, —C(O)O—, —S—, —S(O)—, —S(O)₂—, —S(O)₂N(H)—, —S(O)₂N(C₁₋₄ alkyl)-, —S(O)₂N(C₃₋₅cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, the linker is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, the linker is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In yet other embodiments, the linker comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In yet other embodiments, L is a diradical of a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, the linker is a heteroalkylene having from 4 to 30 atoms selected from carbon, oxygen, nitrogen, and sulfur. In certain embodiments, L is a heteroalkylene having from 4 to 20 atoms selected from carbon, oxygen, nitrogen, and sulfur. In certain embodiments, the linker is a heteroalkylene having from 4 to 10 atoms selected from carbon, oxygen, nitrogen, and sulfur. In certain embodiments, the linker is a heteroalkylene having from 4 to 30 atoms selected from carbon, oxygen, and nitrogen. In certain embodiments, the linker is a heteroalkylene having from 4 to 20 atoms selected from carbon, oxygen, and nitrogen. In certain embodiments, the linker is a heteroalkylene having from 4 to 10 atoms selected from carbon, oxygen, and nitrogen. In certain embodiments, the linker is a heteroalkylene having from 4 to 30 atoms selected from carbon and oxygen. In certain embodiments, the linker is a heteroalkylene having from 4 to 20 atoms selected from carbon and oxygen. In certain embodiments, the linker is a heteroalkylene having from 4 to 10 atoms selected from carbon and oxygen.

In additional embodiments, the linker is an optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group.

In certain embodiments, the linker is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted carbocyclyl, or optionally substituted heterocyclyl, wherein R** represents independently for each occurrence hydrogen, C₁₋₆ alkyl, or C₃₋₆ cycloalkyl.

In certain embodiments, the linker is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms selected from nitrogen, oxygen, and sulfur, wherein R** represents independently for each occurrence hydrogen, C₁₋₆ alkyl, or C₃₋₆ cycloalkyl.

In certain embodiments, the linker has the formula —N(R)-(optionally substituted 3-20 membered heteroalkylene)ₚ-CH₂—C(O)—, wherein R is hydrogen or optionally substituted C₁-C₆ alkyl, and p is 0 or 1.

In certain embodiments, the linker has the formula —N(R)-(3-20 membered heteroalkylene)ₚ-CH₂—C(O)—; wherein the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano; R is hydrogen or optionally substituted C₁-C₆ alkyl; and p is 0 or 1.

In certain embodiments, the linker has the formula —N(R)-(3-20 membered heteroalkylene)ₚ-CH₂—C(O)—; wherein the 3-20 membered heteroalkylene is optionally substituted with 1, 2, or 3 substituents independently selected from halogen and C₁-C₆ haloalkyl; R is hydrogen or C₁-C₆ alkyl; and p is 0 or 1.

In some embodiments, the linker is one of the following:

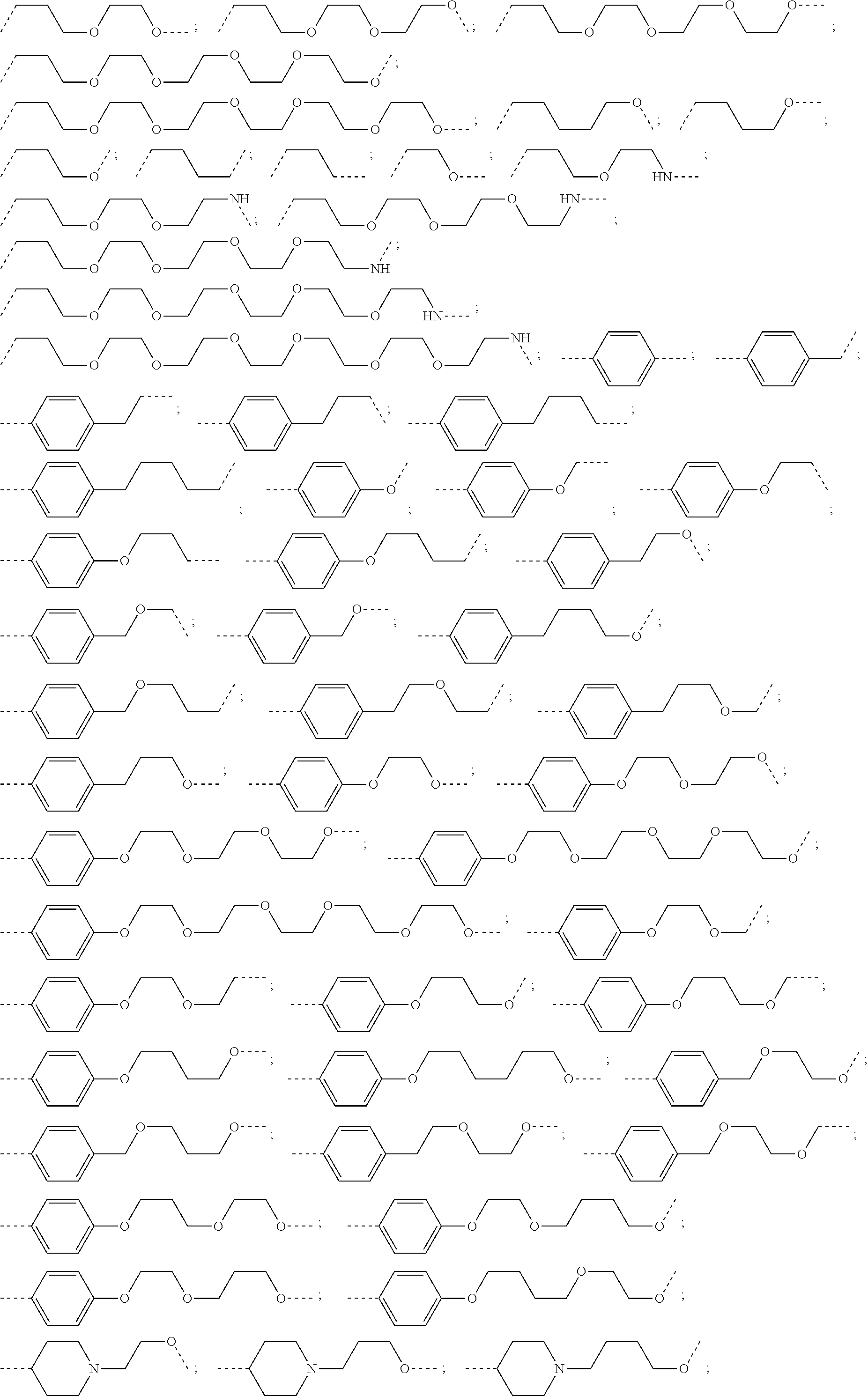

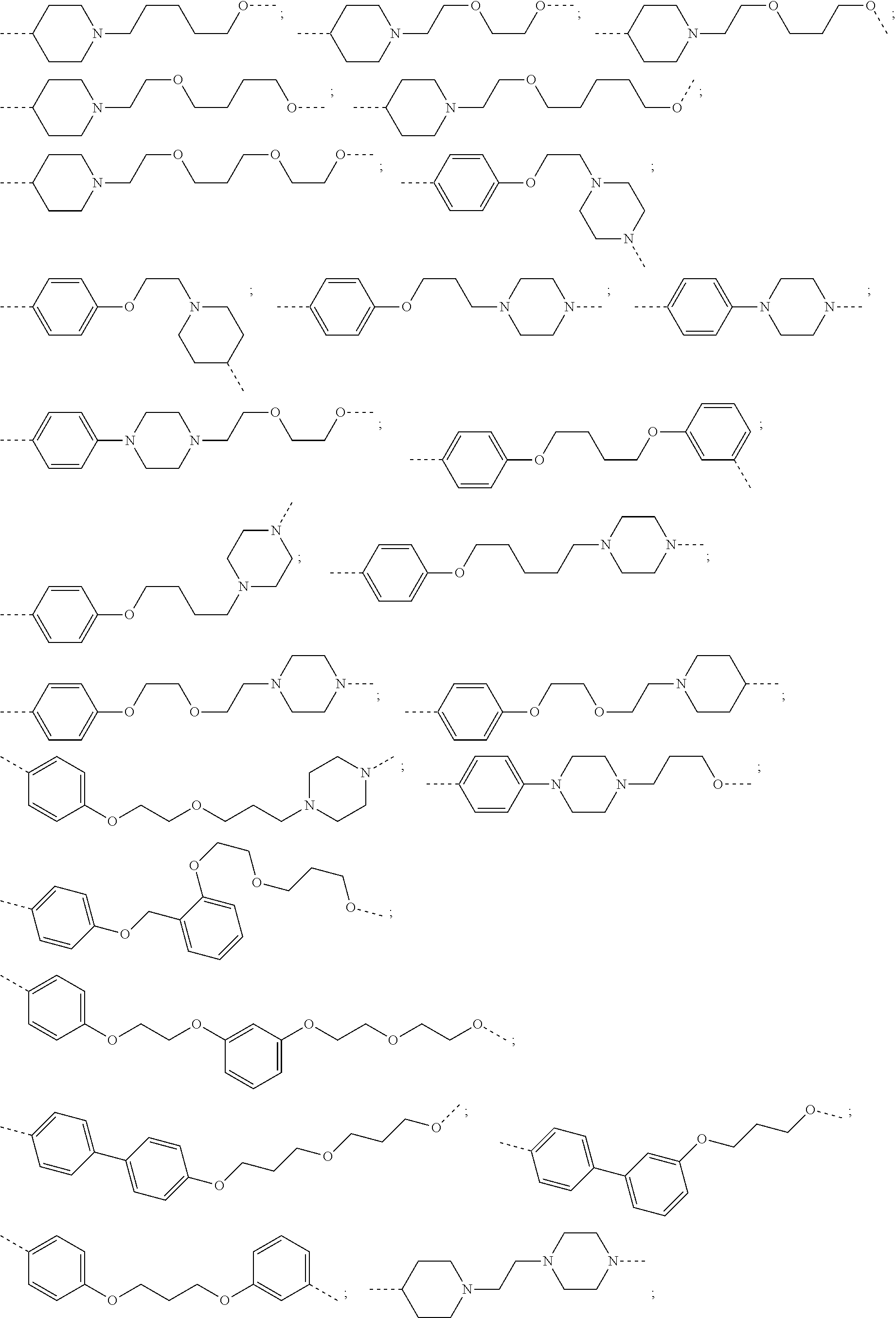

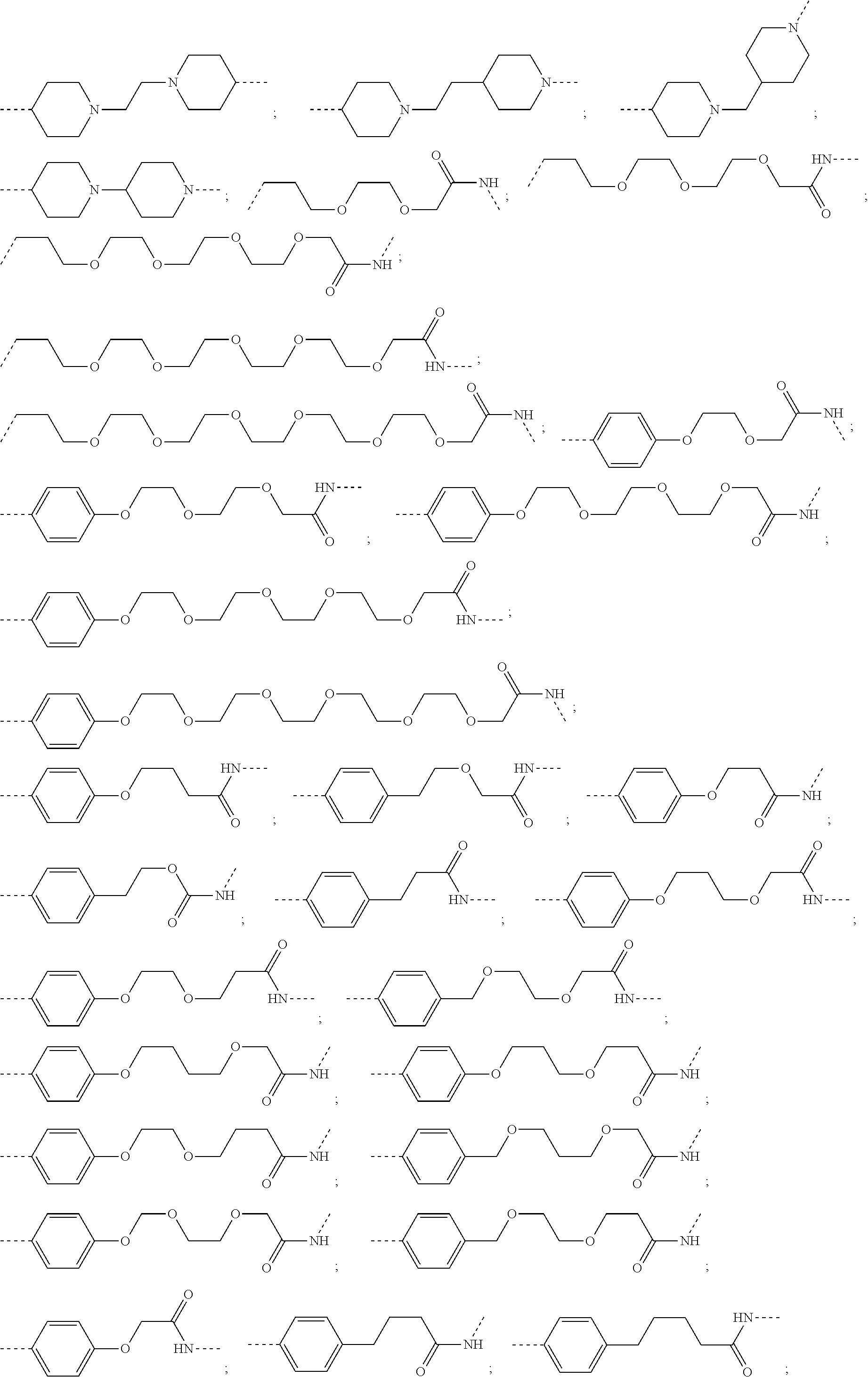

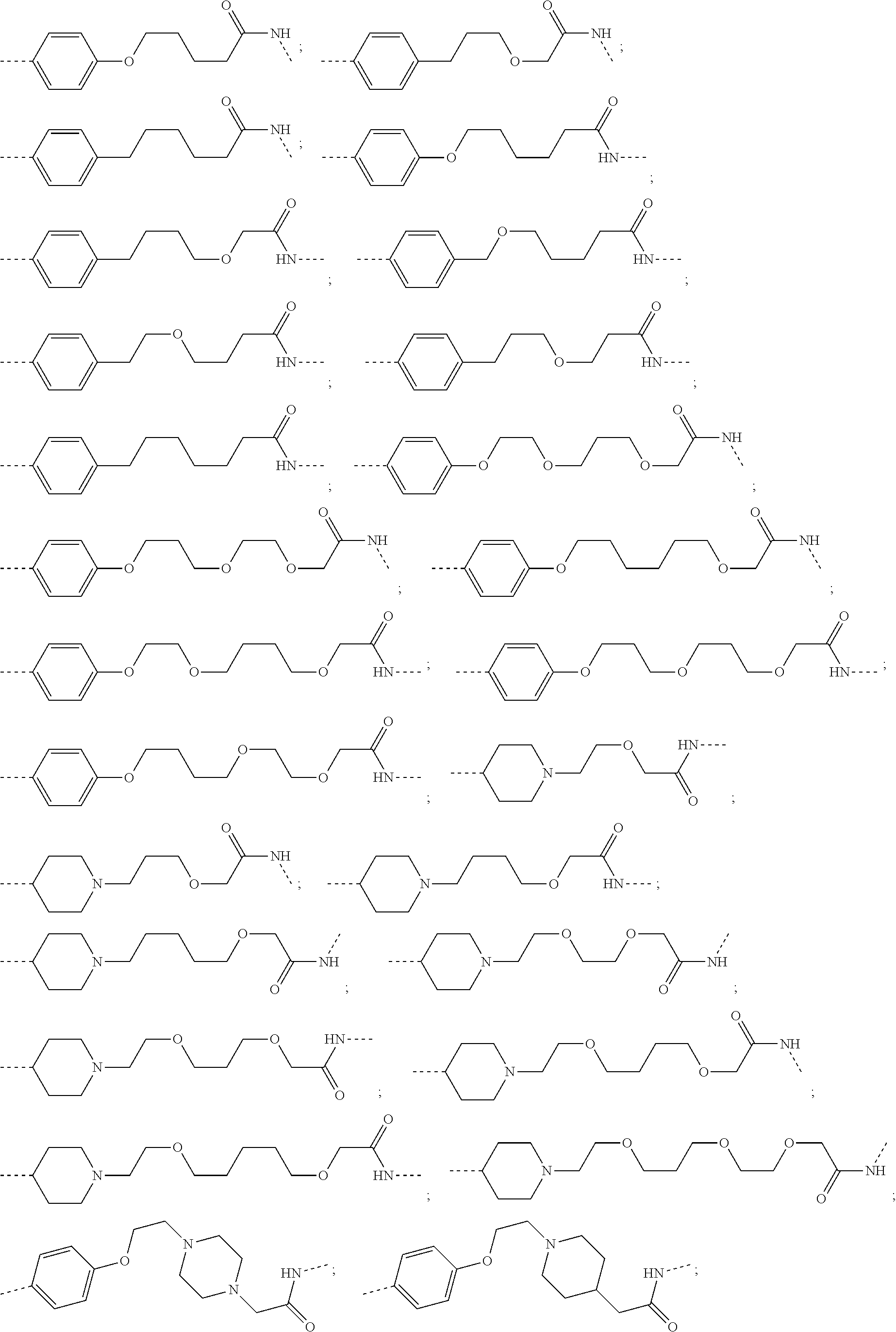

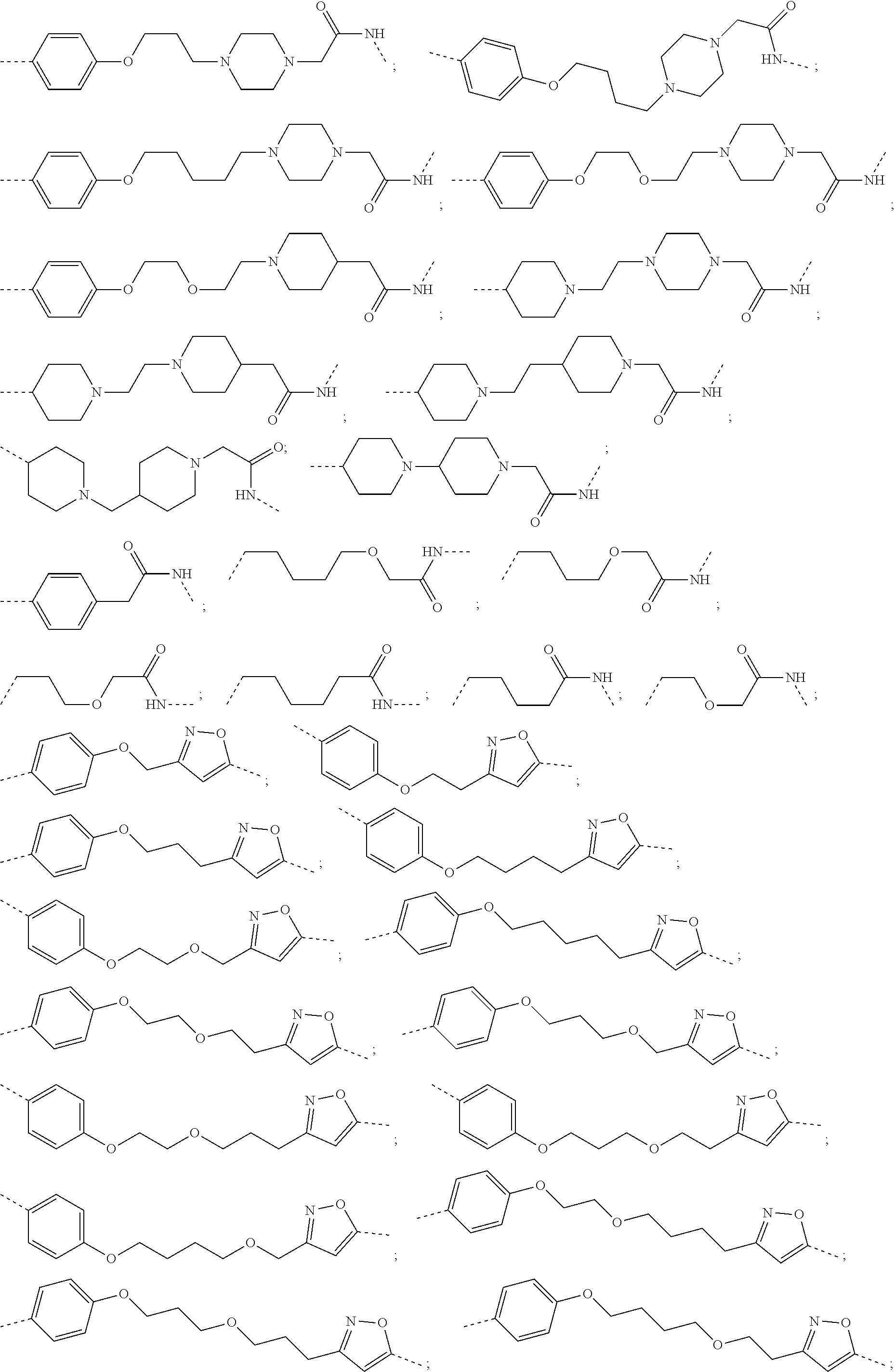

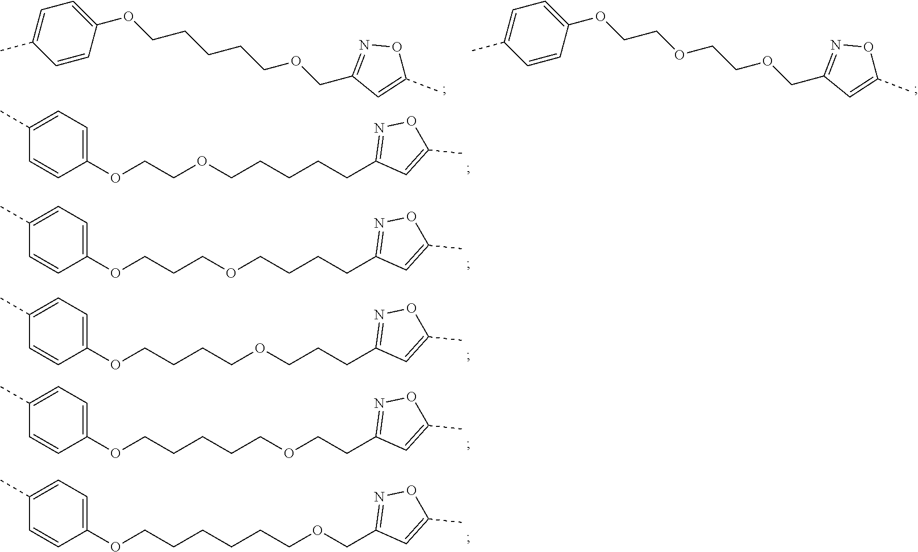

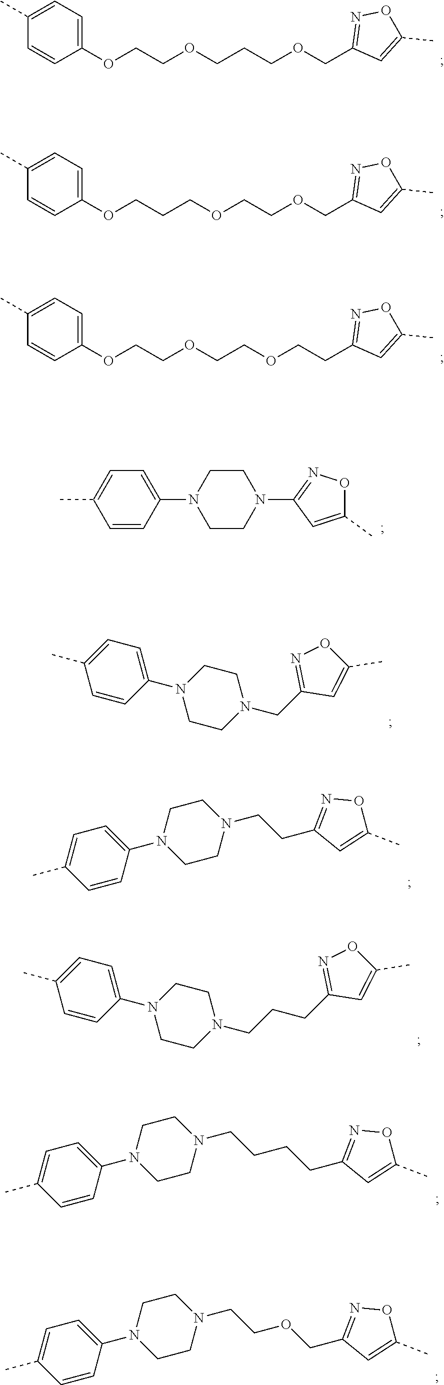

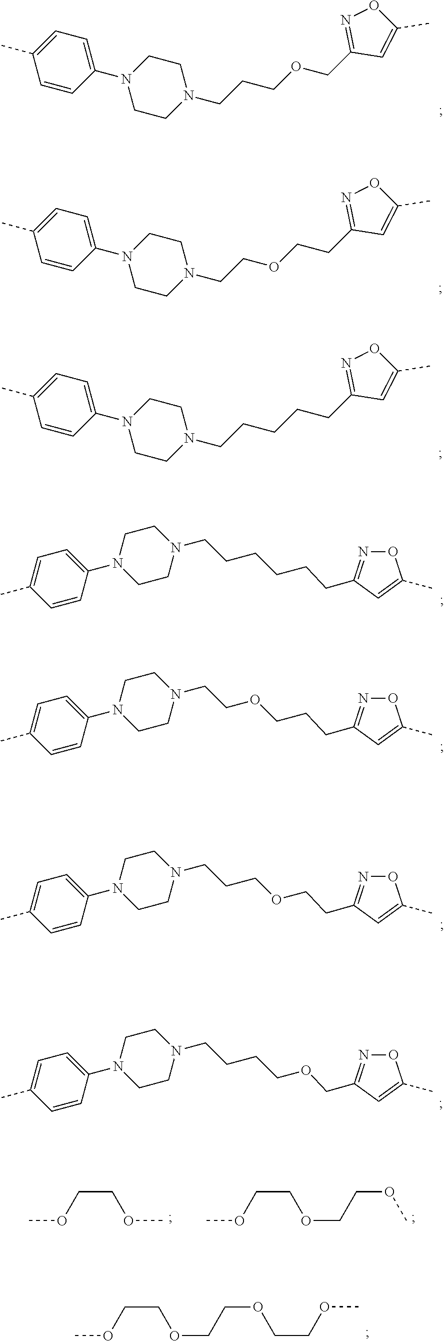

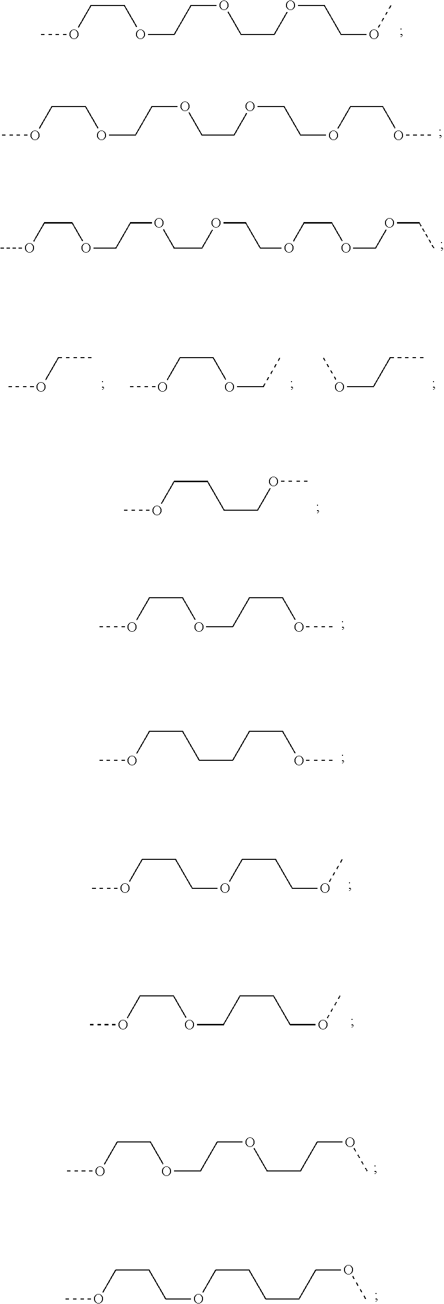

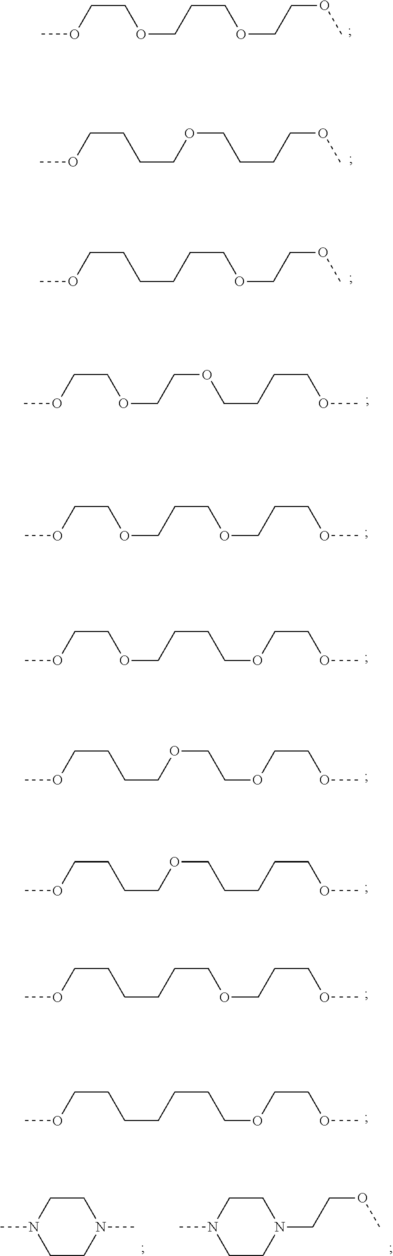

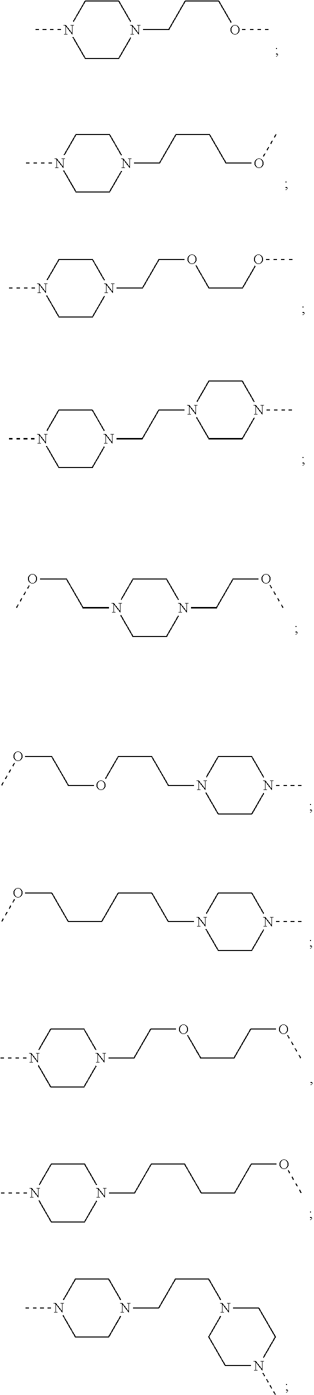

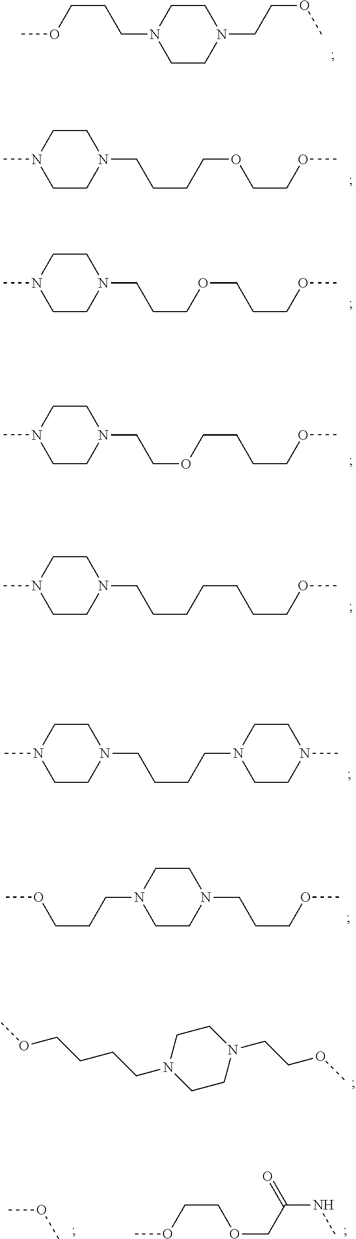

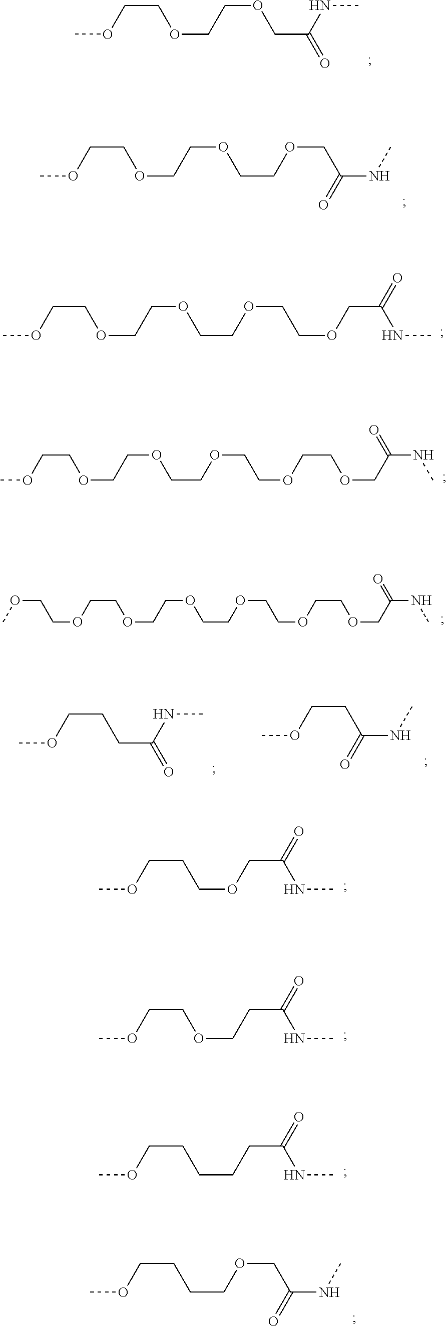

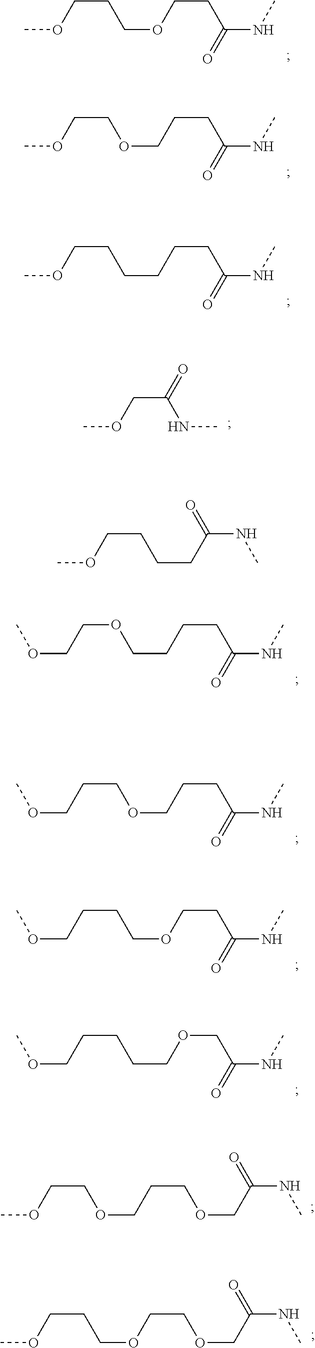

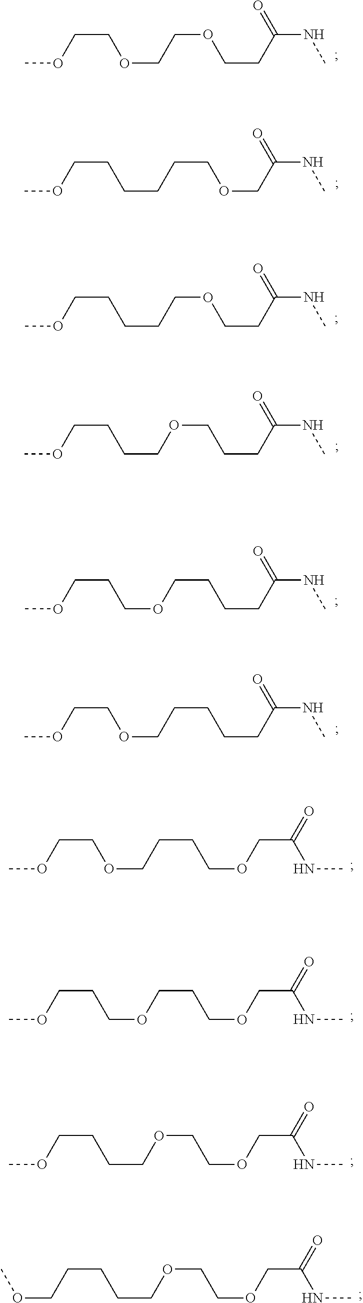

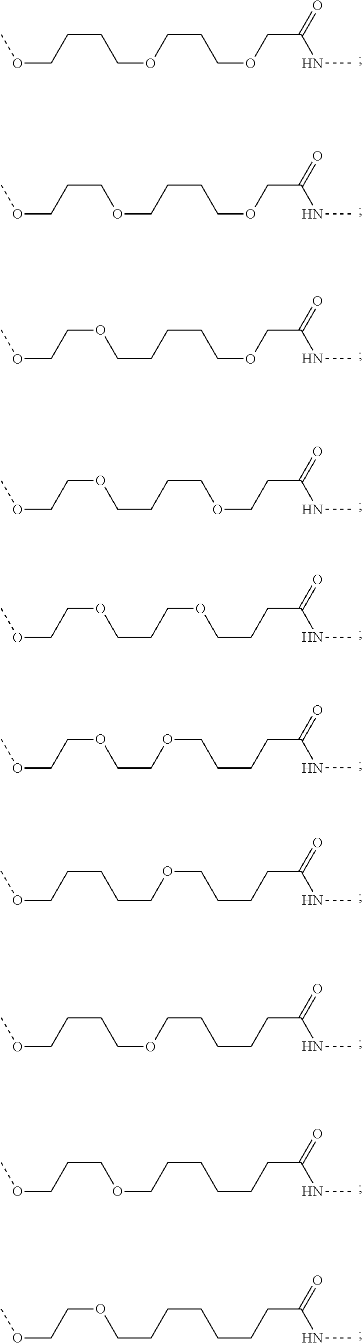

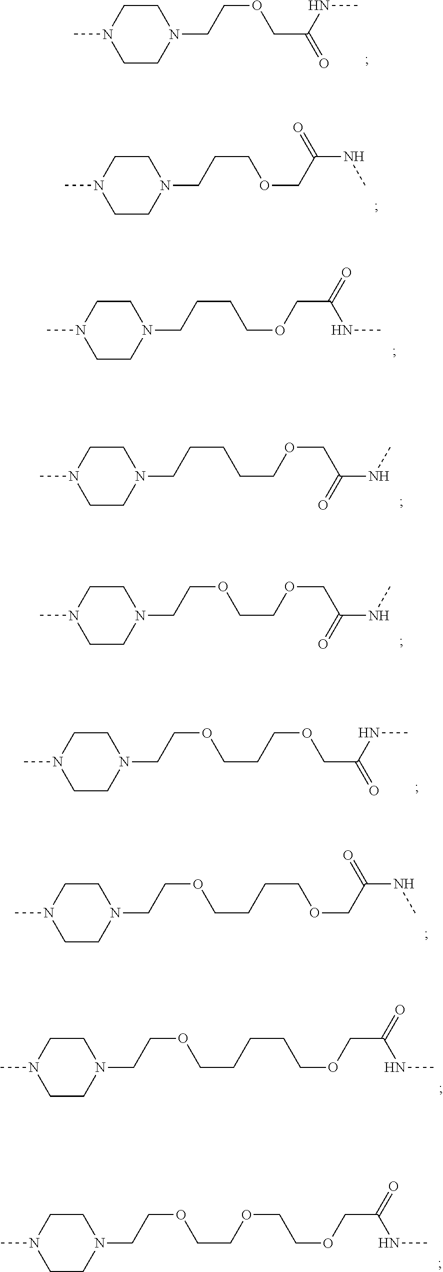

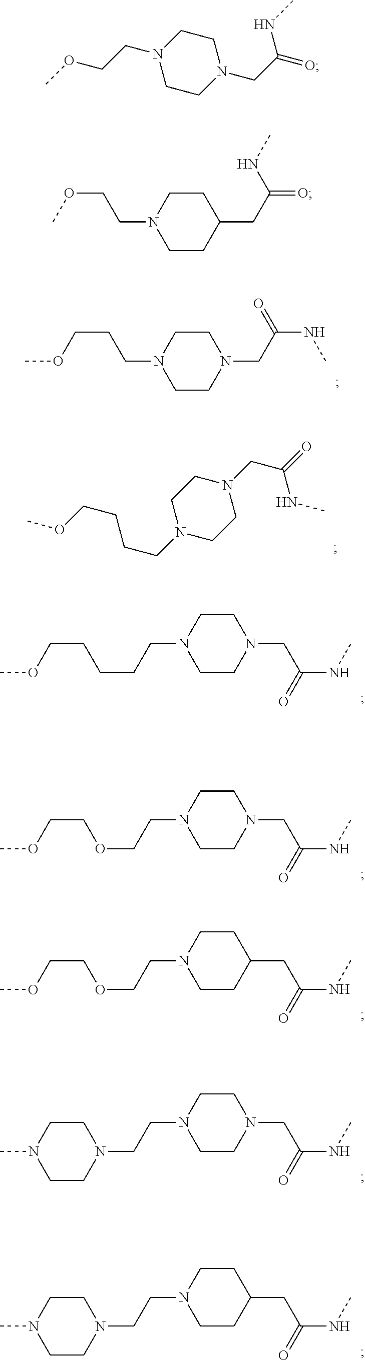

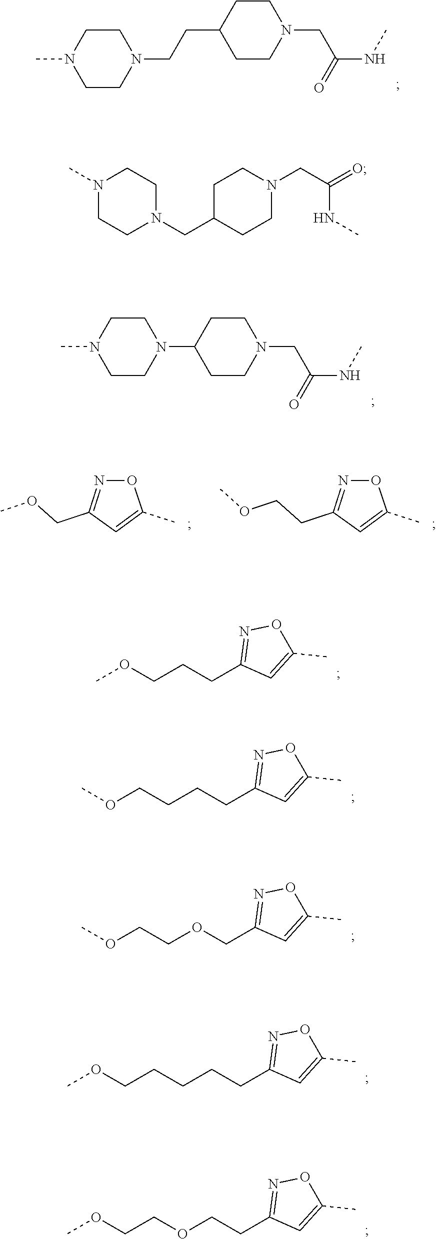

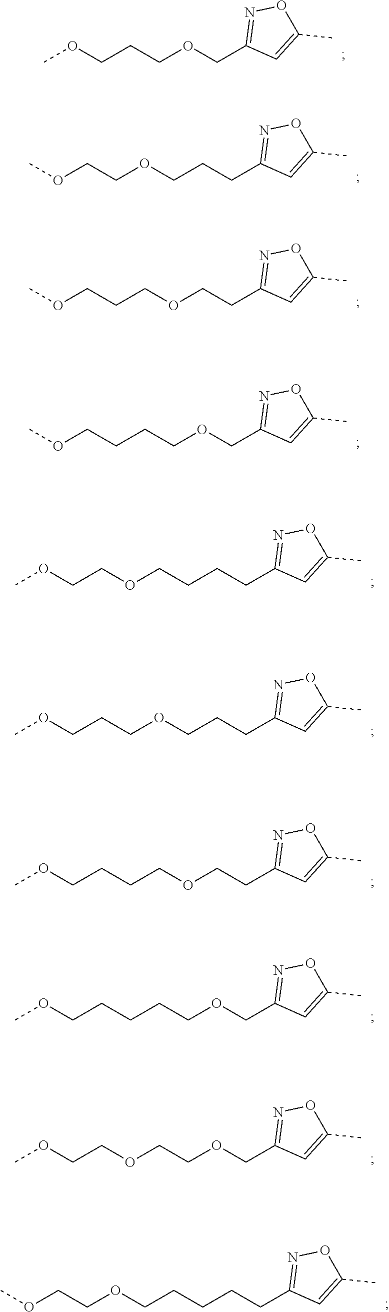

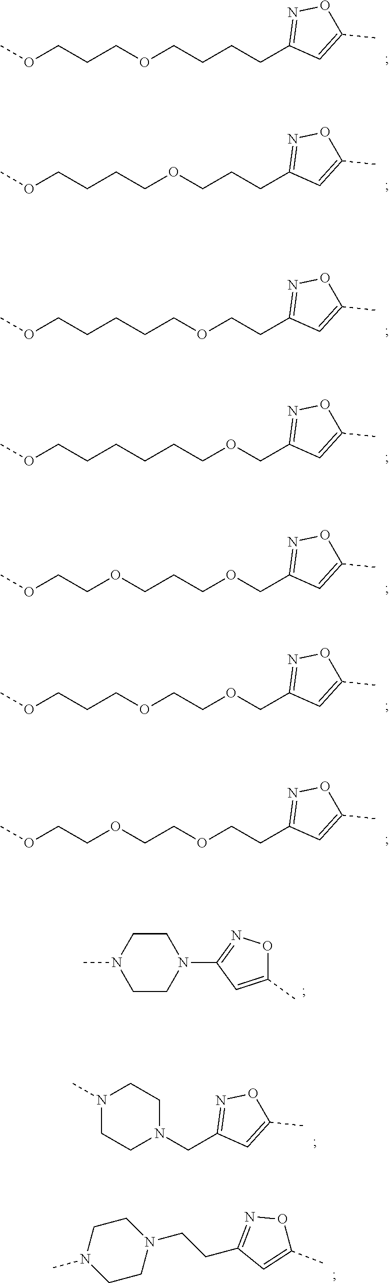

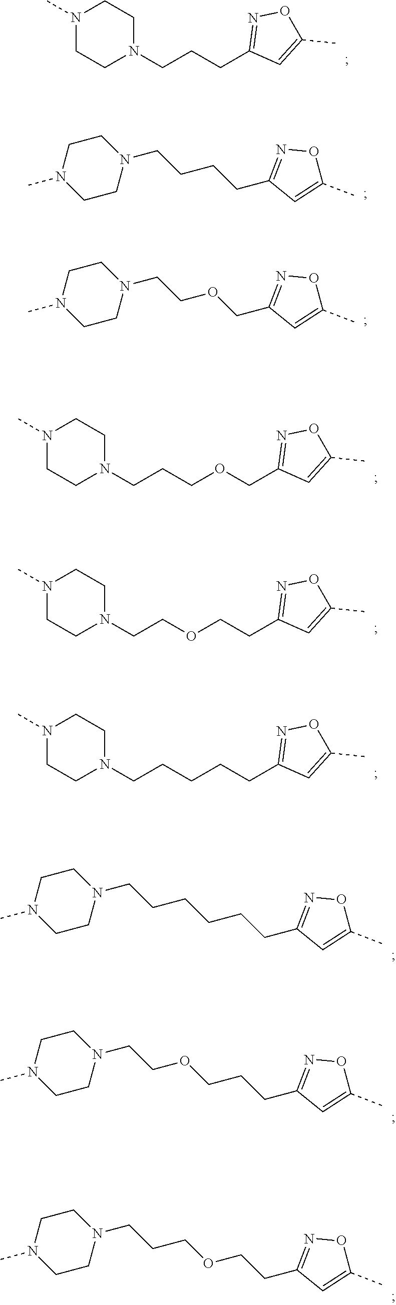

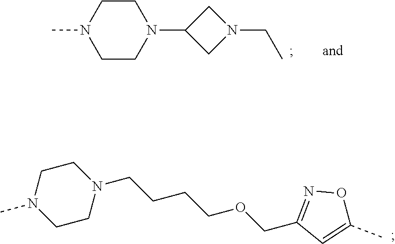

wherein a dashed bond indicates a point of attachment.

In certain embodiments, the linker has the formula —(Co-12 alkylene)-(optionally substituted 3-40 membered heteroalkylene)-(C₀₋₁₂ alkylene)-. In certain embodiments, the linker is C₄₋₁₄ alkylene. In certain embodiments, the linker is —(CH₂)₆₋₁₀—.

### Exemplary More Specific Embodiments

In certain embodiments, the

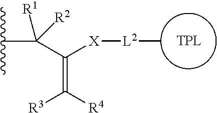

portion of Formula I is one of the following (thereby providing compounds that bind to KRAS):

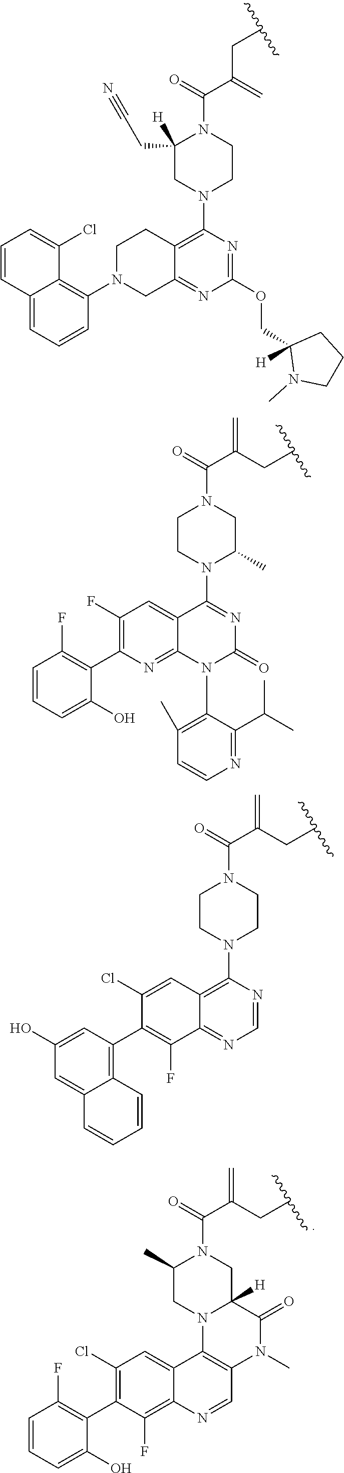

In certain embodiments, the

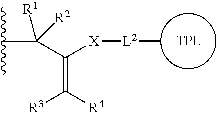

portion of Formula I is one of the following (thereby providing compounds that bind to BTK):

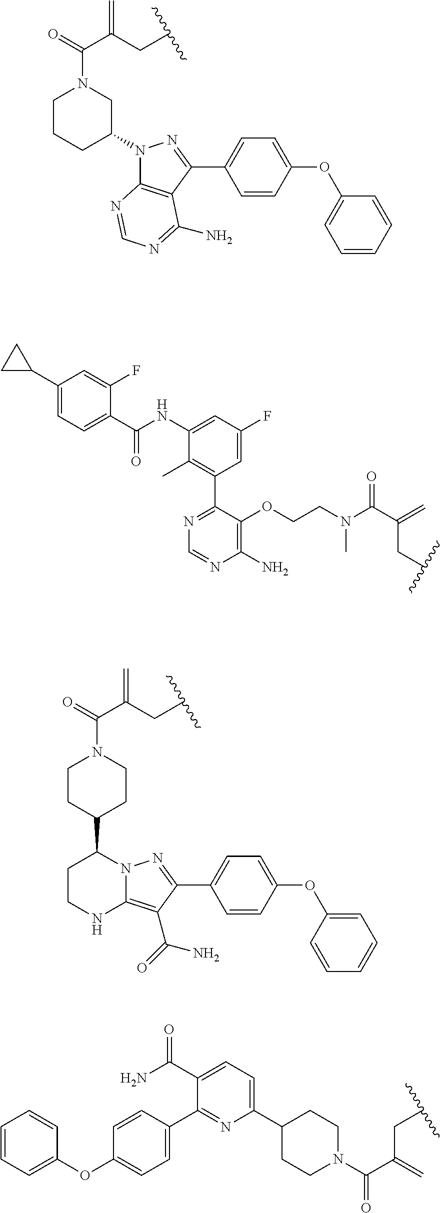 [SMILES: *CC(=C)C(=O)N1CCC[C@@H](n2nc(-c3ccc(Oc4ccccc4)cc3)c3c(N)ncnc32)C1 ; *CC(=C)C(=O)N(C)CCOc1c(N)ncnc1-c1cc(F)cc(NC(=O)c2ccc(C3CC3)cc2F)c1C ; *CC(=C)C(=O)N1CCC([C@@H]2CCNc3c(C(N)=O)c(-c4ccc(Oc5ccccc5)cc4)nn32)CC1 ; *CC(=C)C(=O)N1CCC(c2ccc(C(N)=O)c(-c3ccc(Oc4ccccc4)cc3)n2)CC1]

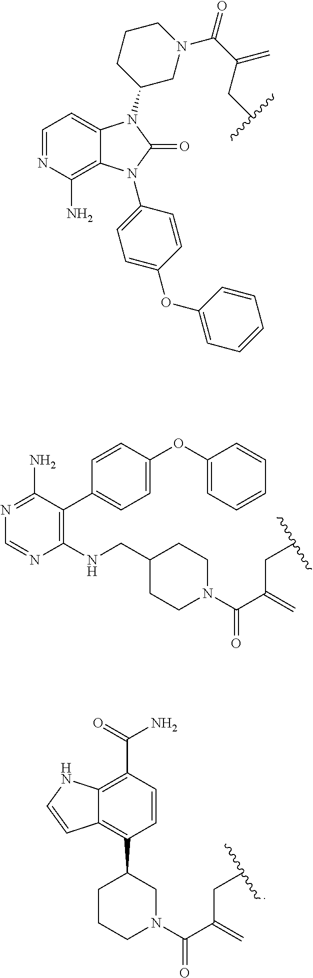 [SMILES: *CC(=C)C(=O)N1CCC[C@@H](n2c(=O)n(-c3ccc(Oc4ccccc4)cc3)c3c(N)nccc32)C1 ; *CC(=C)C(=O)N1CCC(CNc2ncnc(N)c2-c2ccc(Oc3ccccc3)cc2)CC1 ; *CC(=C)C(=O)N1CCC[C@@H](c2ccc(C(N)=O)c3[nH]ccc23)C1]

In certain embodiments, the

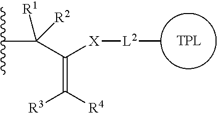

portion of Formula I is one of the following (thereby providing compounds that bind to EGFR):

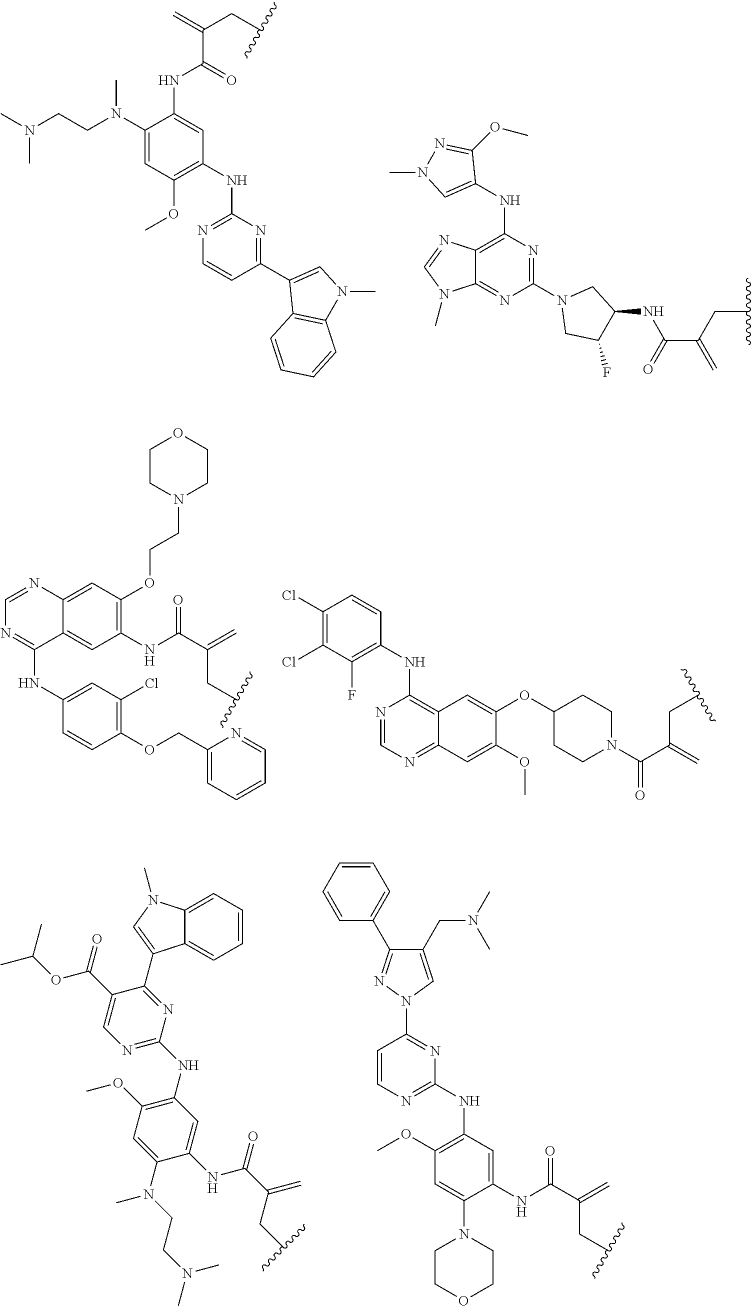

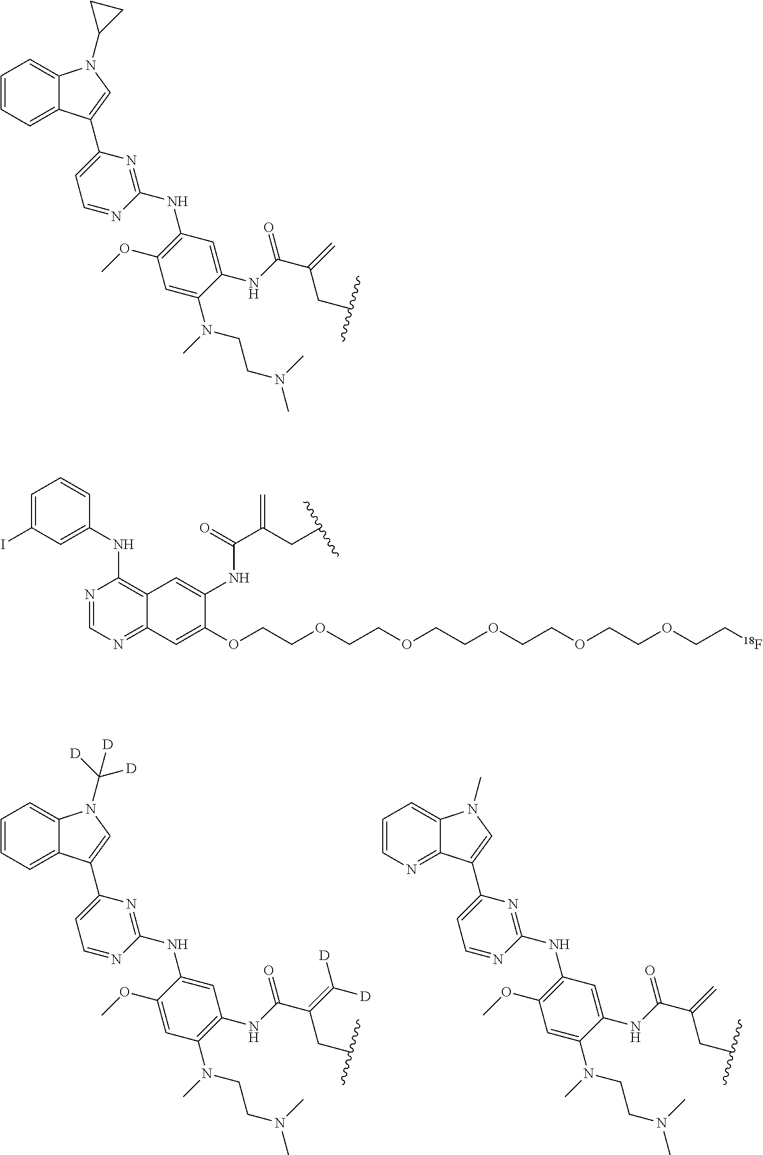 [SMILES: *CC(=C)C(=O)Nc1cc(Nc2nccc(-c3cn(C4CC4)c4ccccc34)n2)c(OC)cc1N(C)CCN(C)C ; *CC(=C)C(=O)Nc1cc2c(Nc3cccc(I)c3)ncnc2cc1OCCOCCOCCOCCOCCOCC[18F] ; *CC(C(=O)Nc1cc(Nc2nccc(-c3cn(C([2H])([2H])[2H])c4ccccc34)n2)c(OC)cc1N(C)CCN(C)C)=C([2H])[2H] ; *CC(=C)C(=O)Nc1cc(Nc2nccc(-c3cn(C)c4cccnc34)n2)c(OC)cc1N(C)CCN(C)C]

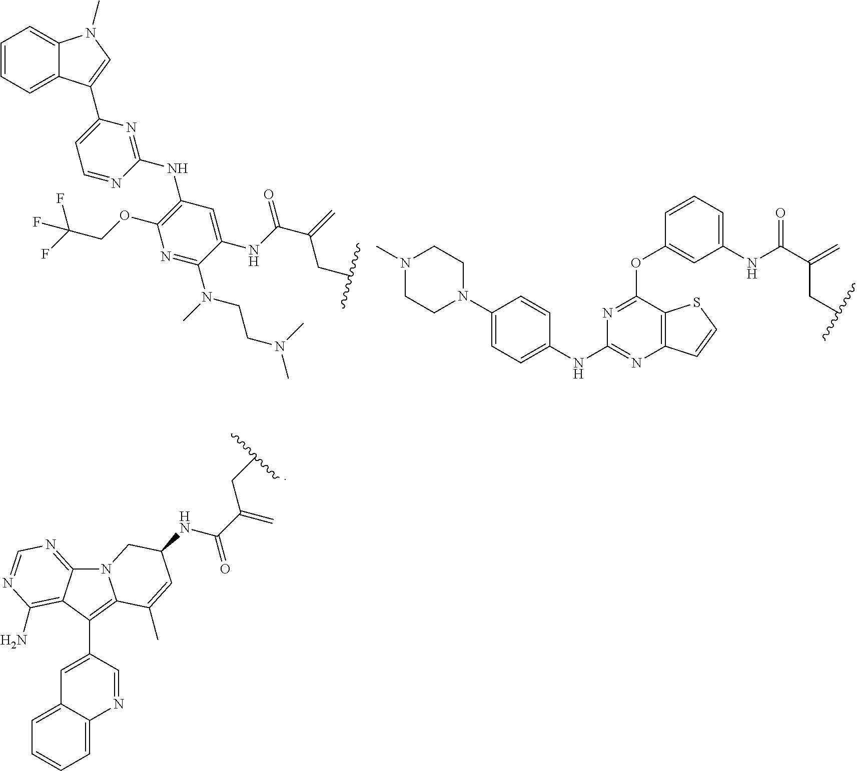

In certain embodiments, the

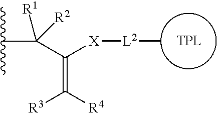

portion of Formula I is one of the following (thereby providing compounds that bind to FGFR1):

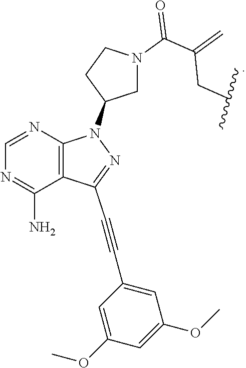 [SMILES: *CC(=C)C(=O)N1CC[C@H](n2nc(C#Cc3cc(OC)cc(OC)c3)c3c(N)ncnc32)C1]

In certain embodiments, the

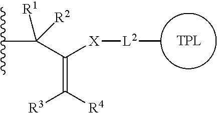

portion of Formula I is one of the following (thereby providing compounds that bind to FGFR4):

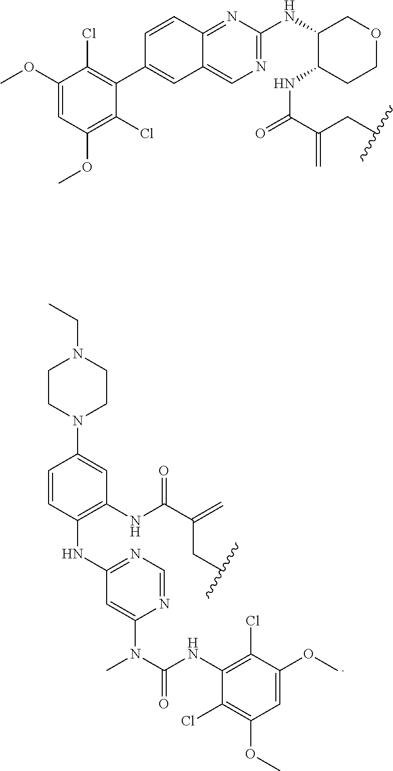 [SMILES: *CC(=C)C(=O)N[C@H]1CCOC[C@H]1Nc1ncc2cc(-c3c(Cl)c(OC)cc(OC)c3Cl)ccc2n1 ; *CC(=C)C(=O)Nc1cc(N2CCN(CC)CC2)ccc1Nc1cc(N(C)C(=O)Nc2c(Cl)c(OC)cc(OC)c2Cl)ncn1]

In certain embodiments, the

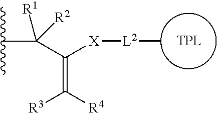

portion of Formula I is one of the following (thereby providing compounds that bind to BTK and/or EGFR):

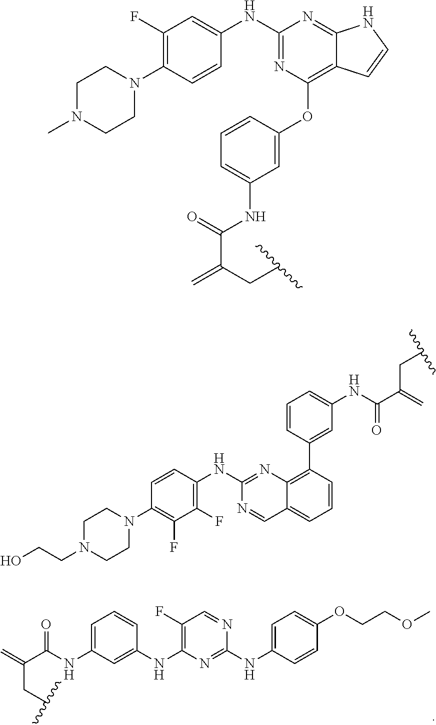

In certain embodiments, the

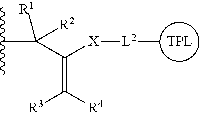

portion of Formula I is one of the following (thereby providing compounds that bind to IDH1):

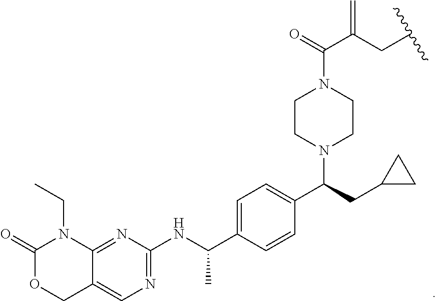 [SMILES: *CC(=C)C(=O)N1CCN([C@@H](CC2CC2)c2ccc([C@H](C)Nc3ncc4c(n3)N(CC)C(=O)OC4)cc2)CC1]

In certain embodiments, the

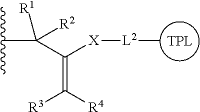

portion of Formula I is one of the following (thereby providing compounds that bind to TBK1, NAK, and/or T2K):

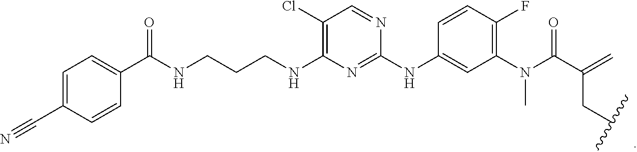 [SMILES: *CC(=C)C(=O)N(C)c1cc(Nc2ncc(Cl)c(NCCCNC(=O)c3ccc(C#N)cc3)n2)ccc1F]

In certain embodiments, the

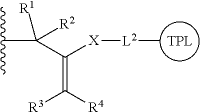

portion of Formula I is one of the following (thereby providing compounds that bind to HER2):

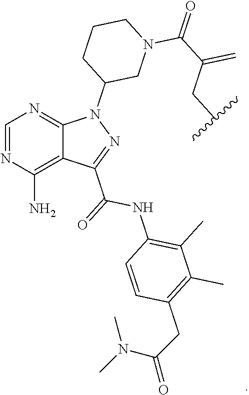 [SMILES: *CC(=C)C(=O)N1CCCC(n2nc(C(=O)Nc3ccc(CC(=O)N(C)C)c(C)c3C)c3c(N)ncnc32)C1]

### Exemplary Specific Compounds

In certain embodiments, the compound is a compound in Table 1, or a pharmaceutically acceptable salt thereof. In certain embodiments, the compound is a compound in Table 1. In certain embodiments, the compound is a compound in Table 1-A, or a pharmaceutically acceptable salt thereof. In certain embodiments, the compound is a compound in Table 1-A.

TABLE 1
Compounds that Bind GSPT1

| No. | Chemical Structure |
|---|---|
| I-1 | 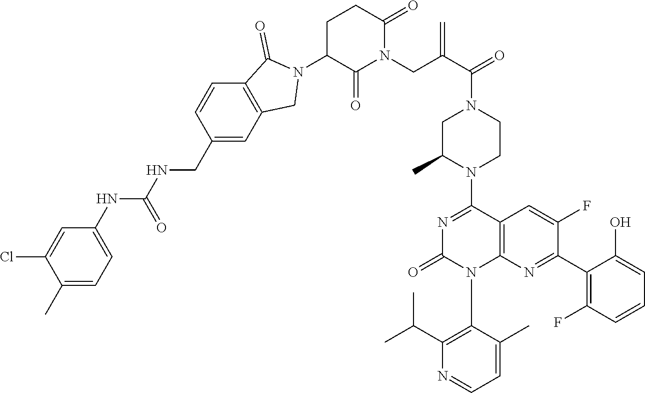 [SMILES: C=C(CN1C(=O)CCC(N2Cc3cc(CNC(=O)Nc4ccc(C)c(Cl)c4)ccc3C2=O)C1=O)C(=O)N1CCN(c2nc(=O)n(-c3c(C)ccnc3C(C)C)c3nc(-c4c(O)cccc4F)c(F)cc23)[C@@H](C)C1] |
| I-2 | 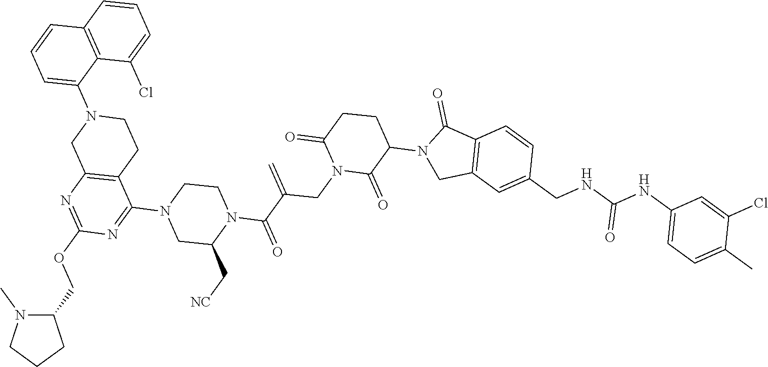 [SMILES: C=C(CN1C(=O)CCC(N2Cc3cc(CNC(=O)Nc4ccc(C)c(Cl)c4)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-3 | 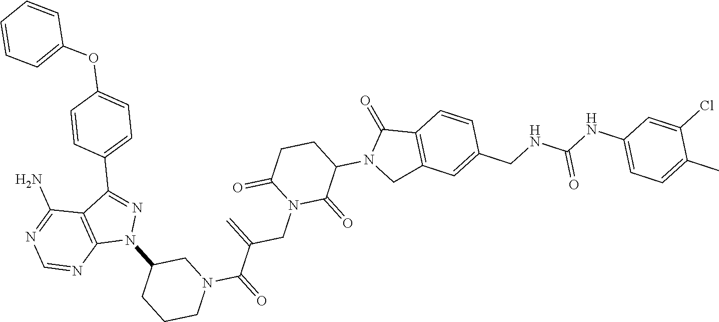 [SMILES: C=C(CN1C(=O)CCC(N2Cc3cc(CNC(=O)Nc4ccc(C)c(Cl)c4)ccc3C2=O)C1=O)C(=O)N1CCC[C@@H](n2nc(-c3ccc(Oc4ccccc4)cc3)c3c(N)ncnc32)C1] |
| I-4 | 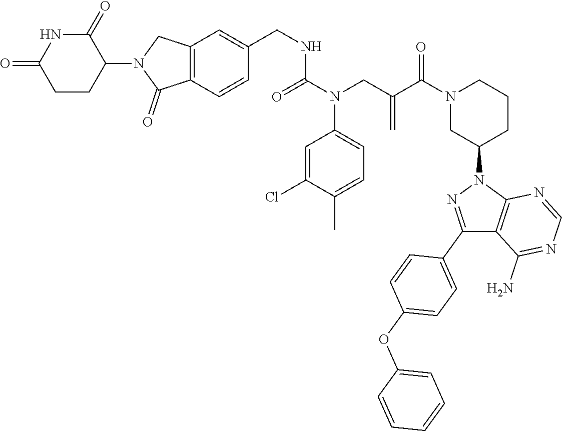 [SMILES: C=C(CN(C(=O)NCc1ccc2c(c1)CN(C1CCC(=O)NC1=O)C2=O)c1ccc(C)c(Cl)c1)C(=O)N1CCC[C@@H](n2nc(-c3ccc(Oc4ccccc4)cc3)c3c(N)ncnc32)C1] |
| I-5 | 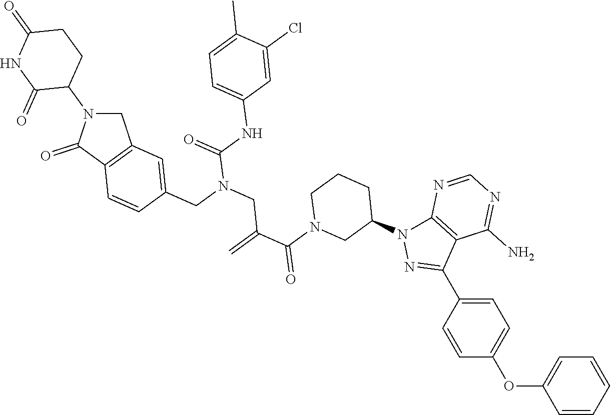 [SMILES: C=C(CN(Cc1ccc2c(c1)CN(C1CCC(=O)NC1=O)C2=O)C(=O)Nc1ccc(C)c(Cl)c1)C(=O)N1CCC[C@@H](n2nc(-c3ccc(Oc4ccccc4)cc3)c3c(N)ncnc32)C1] |
| I-6 | 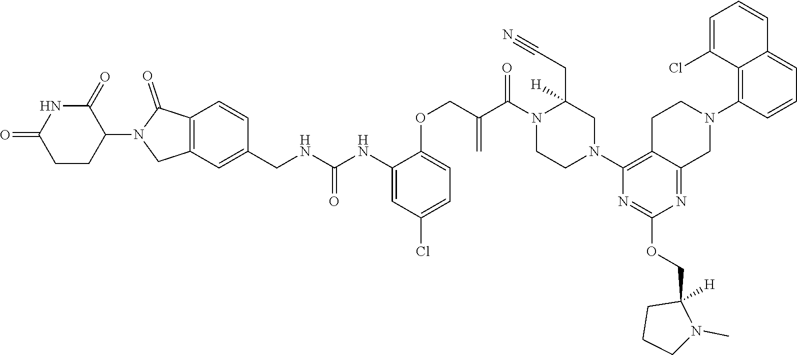 [SMILES: C=C(COc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-7 | 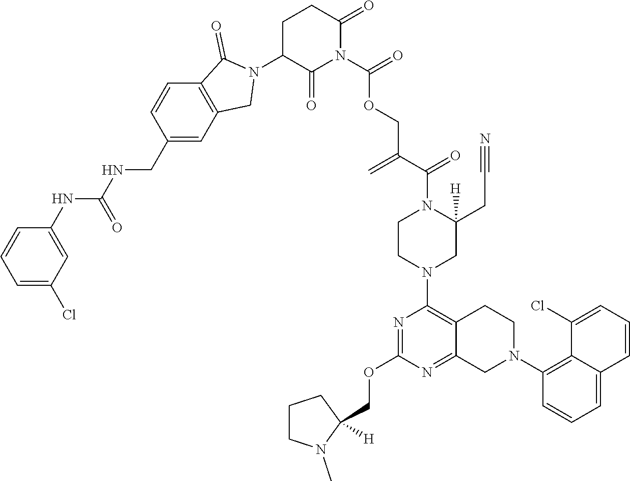 [SMILES: C=C(COC(=O)N1C(=O)CCC(N2Cc3cc(CNC(=O)Nc4cccc(Cl)c4)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-8 | 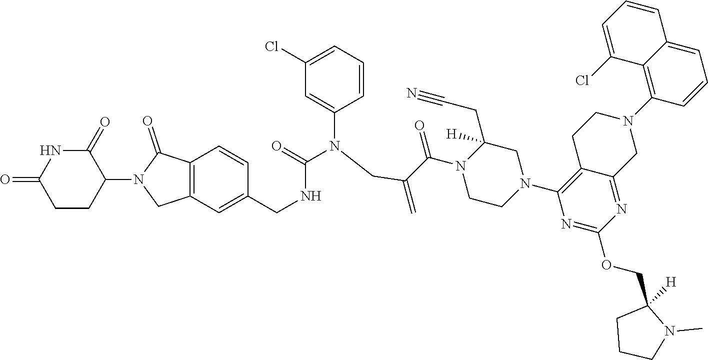 [SMILES: C=C(CN(C(=O)NCc1ccc2c(c1)CN(C1CCC(=O)NC1=O)C2=O)c1cccc(Cl)c1)C(=O)N1CCN(c2nc(OC[C@@H]3CCCN3C)nc3c2CCN(c2cccc4cccc(Cl)c24)C3)C[C@@H]1CC#N] |
| I-9 | 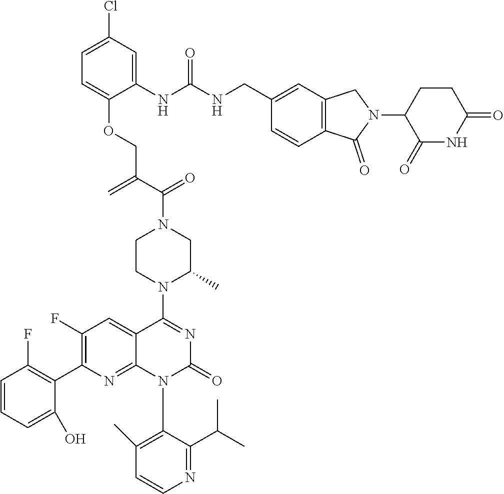 [SMILES: C=C(COc1ccc(Cl)cc1NC(=O)NCc1ccc2c(c1)CN(C1CCC(=O)NC1=O)C2=O)C(=O)N1CCN(c2nc(=O)n(-c3c(C)ccnc3C(C)C)c3nc(-c4c(O)cccc4F)c(F)cc23)[C@@H](C)C1] |
| I-10 | 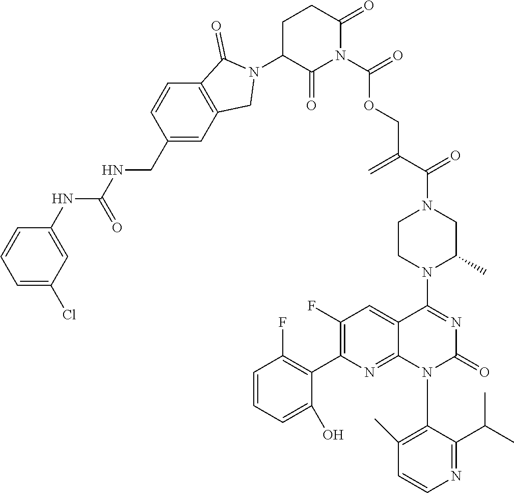 [SMILES: C=C(COC(=O)N1C(=O)CCC(N2Cc3cc(CNC(=O)Nc4cccc(Cl)c4)ccc3C2=O)C1=O)C(=O)N1CCN(c2nc(=O)n(-c3c(C)ccnc3C(C)C)c3nc(-c4c(O)cccc4F)c(F)cc23)[C@@H](C)C1] |
| I-11 | 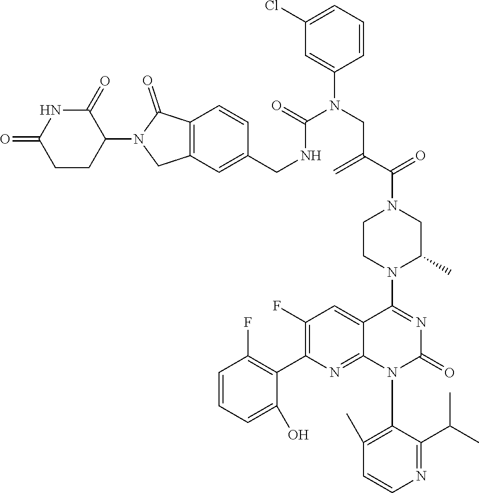 [SMILES: C=C(CN(C(=O)NCc1ccc2c(c1)CN(C1CCC(=O)NC1=O)C2=O)c1cccc(Cl)c1)C(=O)N1CCN(c2nc(=O)n(-c3c(C)ccnc3C(C)C)c3nc(-c4c(O)cccc4F)c(F)cc23)[C@@H](C)C1] |
| I-12 | 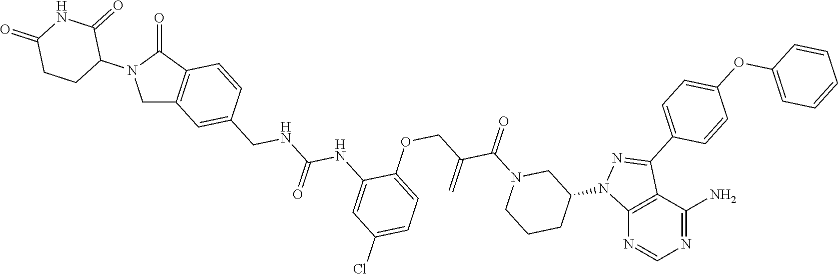 [SMILES: C=C(COc1ccc(Cl)cc1NC(=O)NCc1ccc2c(c1)CN(C1CCC(=O)NC1=O)C2=O)C(=O)N1CCC[C@@H](n2nc(-c3ccc(Oc4ccccc4)cc3)c3c(N)ncnc32)C1] |
| I-13 | 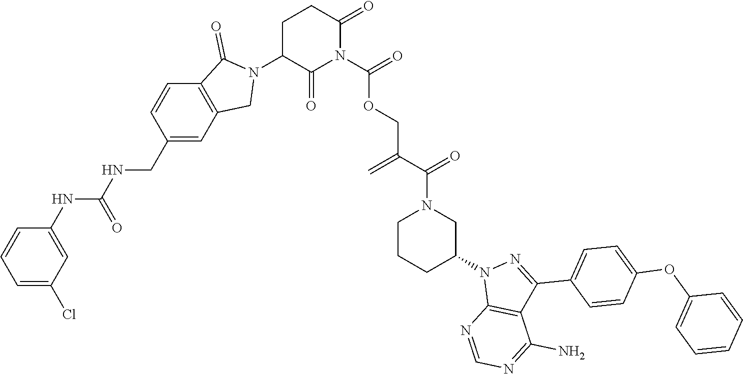 [SMILES: C=C(COC(=O)N1C(=O)CCC(N2Cc3cc(CNC(=O)Nc4cccc(Cl)c4)ccc3C2=O)C1=O)C(=O)N1CCC[C@@H](n2nc(-c3ccc(Oc4ccccc4)cc3)c3c(N)ncnc32)C1] |
| I-14 | 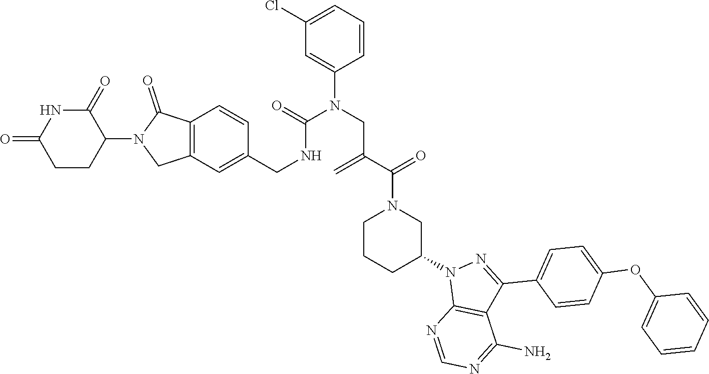 [SMILES: C=C(CN(C(=O)NCc1ccc2c(c1)CN(C1CCC(=O)NC1=O)C2=O)c1cccc(Cl)c1)C(=O)N1CCC[C@@H](n2nc(-c3ccc(Oc4ccccc4)cc3)c3c(N)ncnc32)C1] |
| I-15 | 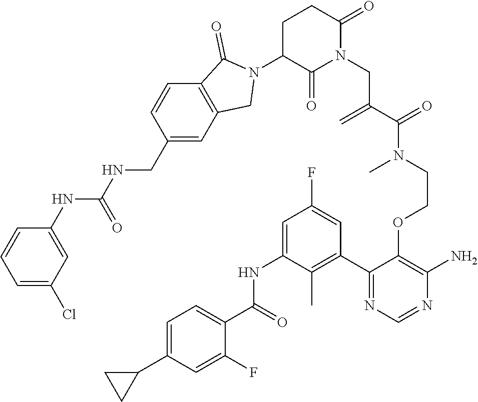 [SMILES: C=C(CN1C(=O)CCC(N2Cc3cc(CNC(=O)Nc4cccc(Cl)c4)ccc3C2=O)C1=O)C(=O)N(C)CCOc1c(N)ncnc1-c1cc(F)cc(NC(=O)c2ccc(C3CC3)cc2F)c1C] |
| I-16 | 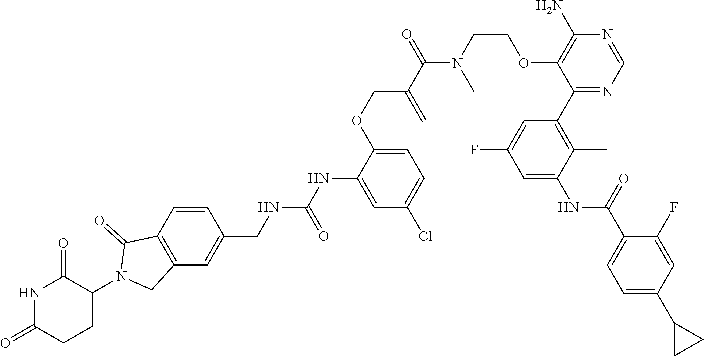 [SMILES: C=C(COc1ccc(Cl)cc1NC(=O)NCc1ccc2c(c1)CN(C1CCC(=O)NC1=O)C2=O)C(=O)N(C)CCOc1c(N)ncnc1-c1cc(F)cc(NC(=O)c2ccc(C3CC3)cc2F)c1C] |
| I-17 | 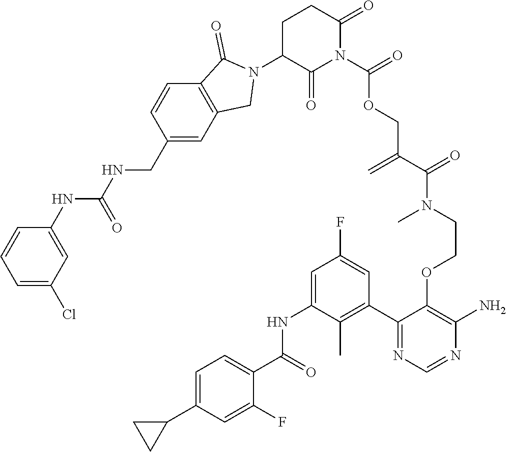 [SMILES: C=C(COC(=O)N1C(=O)CCC(N2Cc3cc(CNC(=O)Nc4cccc(Cl)c4)ccc3C2=O)C1=O)C(=O)N(C)CCOc1c(N)ncnc1-c1cc(F)cc(NC(=O)c2ccc(C3CC3)cc2F)c1C] |
| I-18 | 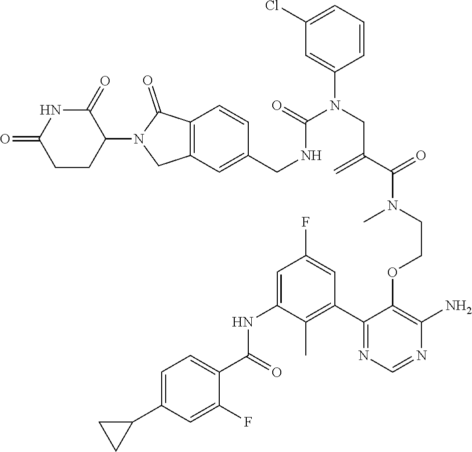 [SMILES: C=C(CN(C(=O)NCc1ccc2c(c1)CN(C1CCC(=O)NC1=O)C2=O)c1cccc(Cl)c1)C(=O)N(C)CCOc1c(N)ncnc1-c1cc(F)cc(NC(=O)c2ccc(C3CC3)cc2F)c1C] |
| I-19 | 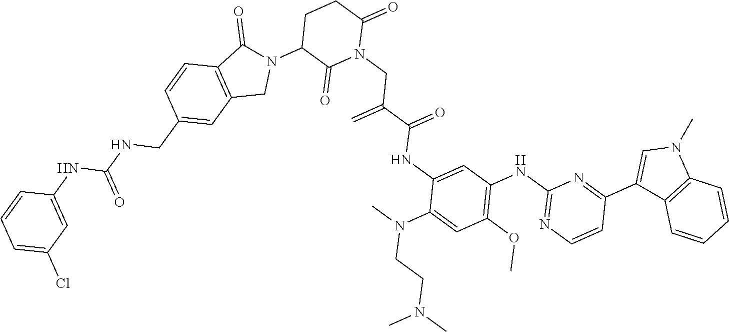 [SMILES: C=C(CN1C(=O)CCC(N2Cc3cc(CNC(=O)Nc4cccc(Cl)c4)ccc3C2=O)C1=O)C(=O)Nc1cc(Nc2nccc(-c3cn(C)c4ccccc34)n2)c(OC)cc1N(C)CCN(C)C] |
| I-20 | 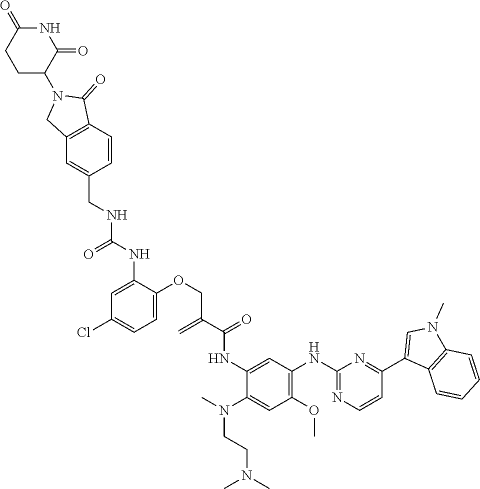 [SMILES: C=C(COc1ccc(Cl)cc1NC(=O)NCc1ccc2c(c1)CN(C1CCC(=O)NC1=O)C2=O)C(=O)Nc1cc(Nc2nccc(-c3cn(C)c4ccccc34)n2)c(OC)cc1N(C)CCN(C)C] |
| I-21 | 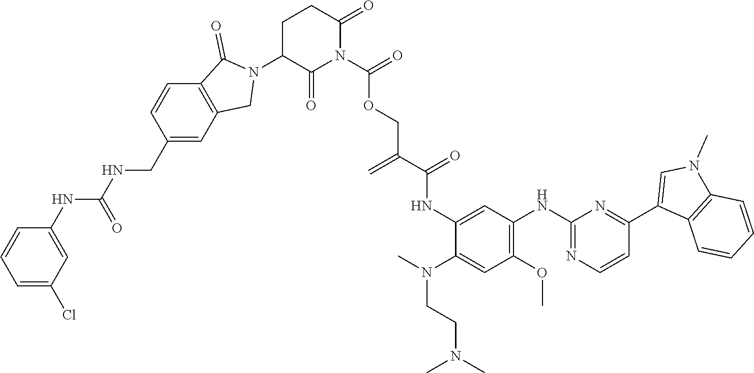 [SMILES: C=C(COC(=O)N1C(=O)CCC(N2Cc3cc(CNC(=O)Nc4cccc(Cl)c4)ccc3C2=O)C1=O)C(=O)Nc1cc(Nc2nccc(-c3cn(C)c4ccccc34)n2)c(OC)cc1N(C)CCN(C)C] |
| I-22 | 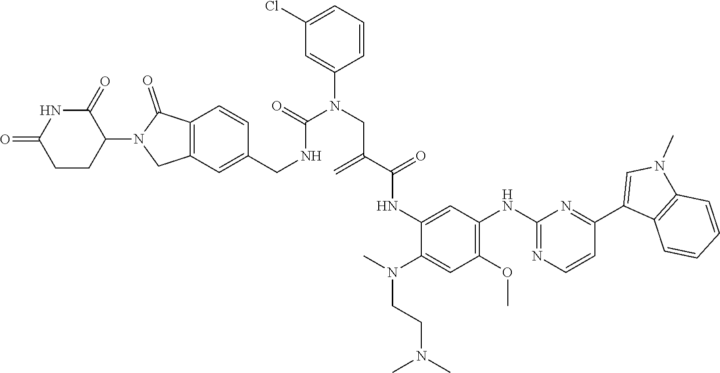 [SMILES: C=C(CN(C(=O)NCc1ccc2c(c1)CN(C1CCC(=O)NC1=O)C2=O)c1cccc(Cl)c1)C(=O)Nc1cc(Nc2nccc(-c3cn(C)c4ccccc34)n2)c(OC)cc1N(C)CCN(C)C] |
| I-23 | 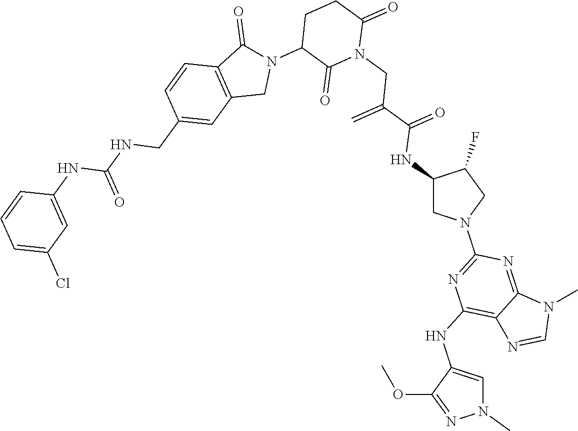 [SMILES: C=C(CN1C(=O)CCC(N2Cc3cc(CNC(=O)Nc4cccc(Cl)c4)ccc3C2=O)C1=O)C(=O)N[C@@H]1CN(c2nc(Nc3cn(C)nc3OC)c3ncn(C)c3n2)C[C@H]1F] |
| I-24 | 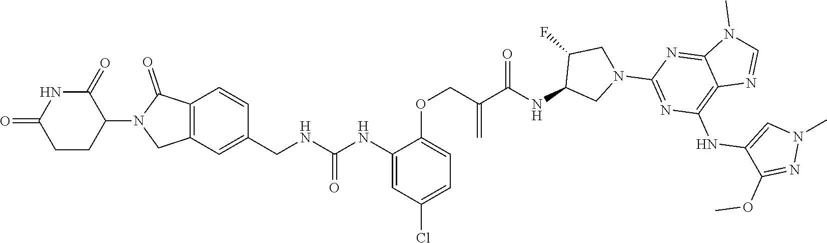 [SMILES: C=C(COc1ccc(Cl)cc1NC(=O)NCc1ccc2c(c1)CN(C1CCC(=O)NC1=O)C2=O)C(=O)N[C@@H]1CN(c2nc(Nc3cn(C)nc3OC)c3ncn(C)c3n2)C[C@H]1F] |
| I-25 | 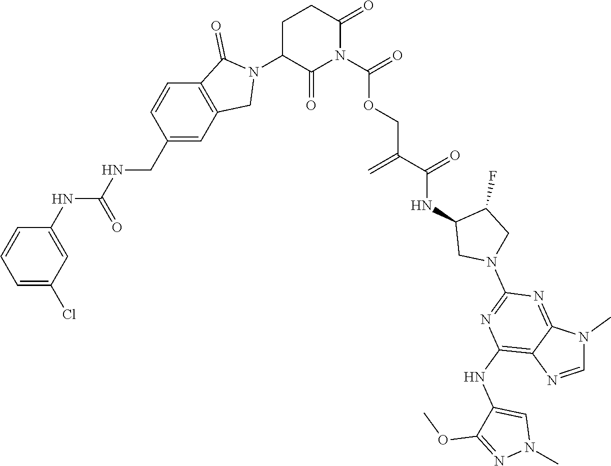 [SMILES: C=C(COC(=O)N1C(=O)CCC(N2Cc3cc(CNC(=O)Nc4cccc(Cl)c4)ccc3C2=O)C1=O)C(=O)N[C@@H]1CN(c2nc(Nc3cn(C)nc3OC)c3ncn(C)c3n2)C[C@H]1F] |
| I-26 | 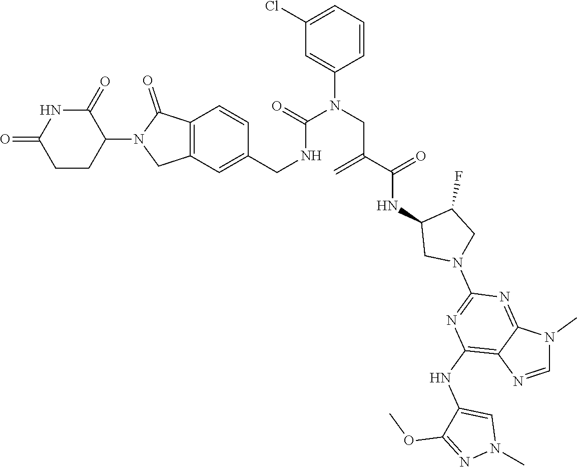 [SMILES: C=C(CN(C(=O)NCc1ccc2c(c1)CN(C1CCC(=O)NC1=O)C2=O)c1cccc(Cl)c1)C(=O)N[C@@H]1CN(c2nc(Nc3cn(C)nc3OC)c3ncn(C)c3n2)C[C@H]1F] |

TABLE 1-A
Additional Compounds that Bind GSPT1

| No. | Chemical Structure |
|---|---|
| I-30 | 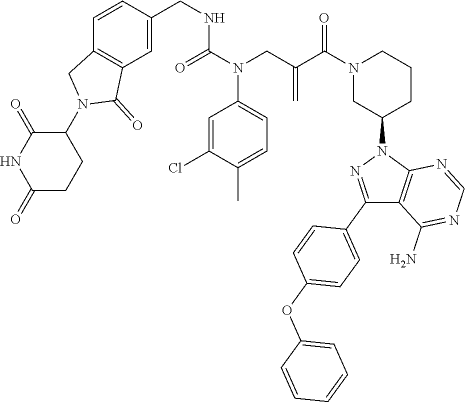 [SMILES: C=C(CN(C(=O)NCc1ccc2c(c1)C(=O)N(C1CCC(=O)NC1=O)C2)c1ccc(C)c(Cl)c1)C(=O)N1CCC[C@@H](n2nc(-c3ccc(Oc4ccccc4)cc3)c3c(N)ncnc32)C1] |
| I-31 | 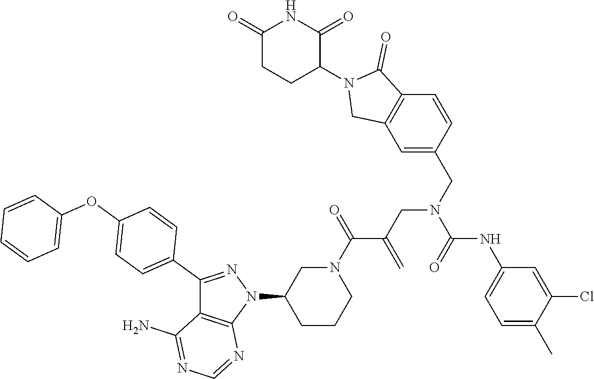 [SMILES: C=C(CN(Cc1ccc2c(c1)CN(C1CCC(=O)NC1=O)C2=O)C(=O)Nc1ccc(C)c(Cl)c1)C(=O)N1CCC[C@@H](n2nc(-c3ccc(Oc4ccccc4)cc3)c3c(N)ncnc32)C1] |
| I-32 | 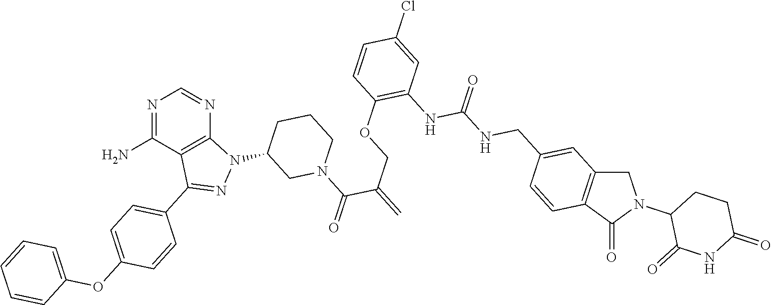 [SMILES: C=C(COc1ccc(Cl)cc1NC(=O)NCc1ccc2c(c1)CN(C1CCC(=O)NC1=O)C2=O)C(=O)N1CCC[C@@H](n2nc(-c3ccc(Oc4ccccc4)cc3)c3c(N)ncnc32)C1] |
| I-33 | 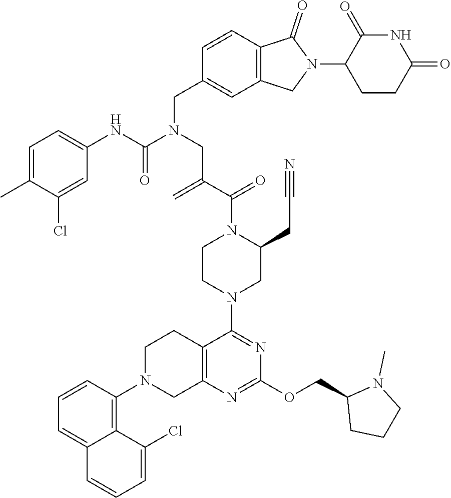 [SMILES: C=C(CN(Cc1ccc2c(c1)CN(C1CCC(=O)NC1=O)C2=O)C(=O)Nc1ccc(C)c(Cl)c1)C(=O)N1CCN(c2nc(OC[C@@H]3CCCN3C)nc3c2CCN(c2cccc4cccc(Cl)c24)C3)C[C@@H]1CC#N] |
| I-34 | 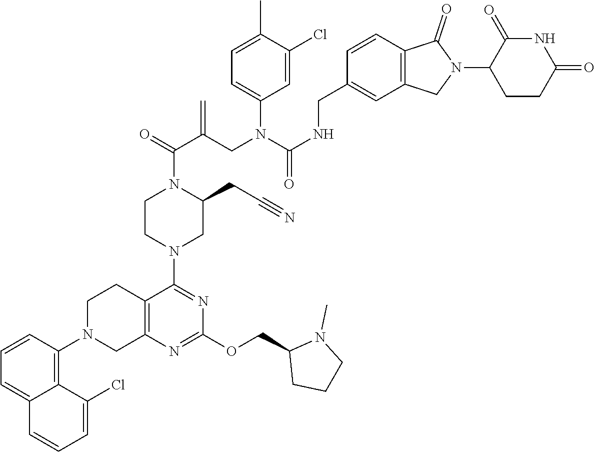 [SMILES: C=C(CN(C(=O)NCc1ccc2c(c1)CN(C1CCC(=O)NC1=O)C2=O)c1ccc(C)c(Cl)c1)C(=O)N1CCN(c2nc(OC[C@@H]3CCCN3C)nc3c2CCN(c2cccc4cccc(Cl)c24)C3)C[C@@H]1CC#N] |
| I-35 | 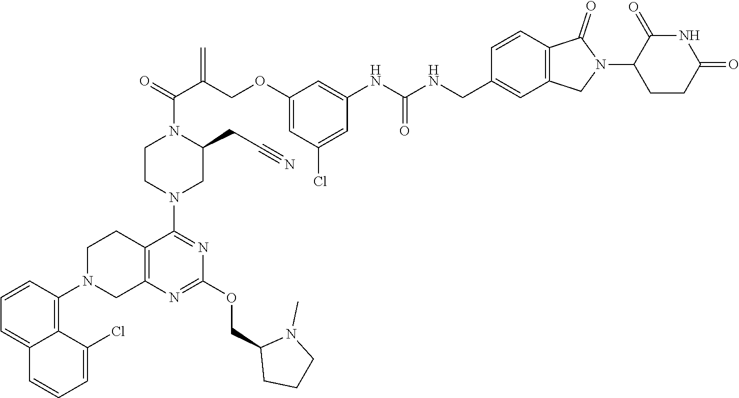 [SMILES: C=C(COc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-36 | 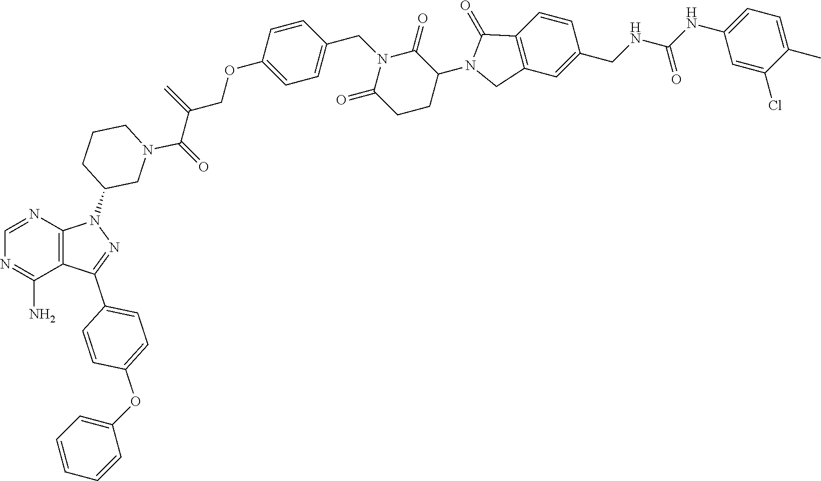 [SMILES: C=C(COc1ccc(CN2C(=O)CCC(N3Cc4cc(CNC(=O)Nc5ccc(C)c(Cl)c5)ccc4C3=O)C2=O)cc1)C(=O)N1CCC[C@@H](n2nc(-c3ccc(Oc4ccccc4)cc3)c3c(N)ncnc32)C1] |
| I-37 | 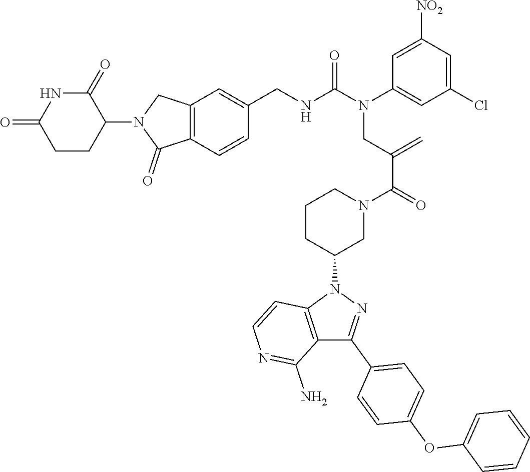 [SMILES: C=C(CN(C(=O)NCc1ccc2c(c1)CN(C1CCC(=O)NC1=O)C2=O)c1cc(Cl)cc([N+](=O)[O-])c1)C(=O)N1CCC[C@@H](n2nc(-c3ccc(Oc4ccccc4)cc3)c3c(N)nccc32)C1] |
| I-38 | 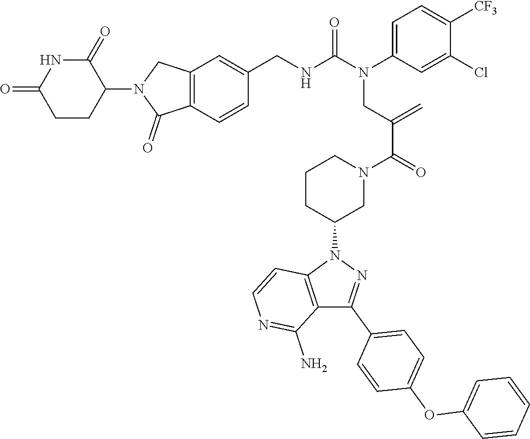 [SMILES: C=C(CN(C(=O)NCc1ccc2c(c1)CN(C1CCC(=O)NC1=O)C2=O)c1ccc(C(F)(F)F)c(Cl)c1)C(=O)N1CCC[C@@H](n2nc(-c3ccc(Oc4ccccc4)cc3)c3c(N)nccc32)C1] |
| I-39 | 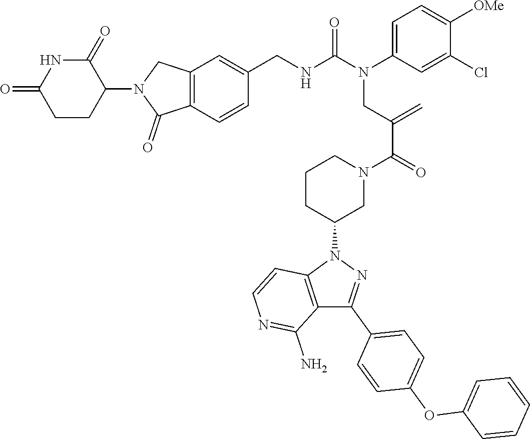 [SMILES: C=C(CN(C(=O)NCc1ccc2c(c1)CN(C1CCC(=O)NC1=O)C2=O)c1ccc(OC)c(Cl)c1)C(=O)N1CCC[C@@H](n2nc(-c3ccc(Oc4ccccc4)cc3)c3c(N)nccc32)C1] |
| I-40 | 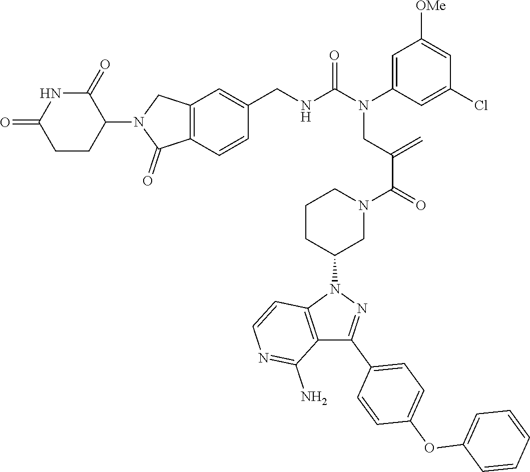 [SMILES: C=C(CN(C(=O)NCc1ccc2c(c1)CN(C1CCC(=O)NC1=O)C2=O)c1cc(Cl)cc(OC)c1)C(=O)N1CCC[C@@H](n2nc(-c3ccc(Oc4ccccc4)cc3)c3c(N)nccc32)C1] |
| I-41 | 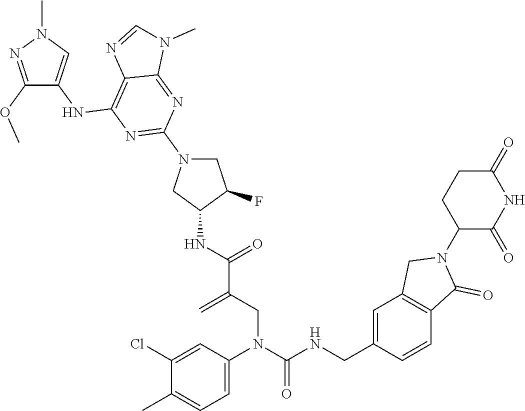 [SMILES: C=C(CN(C(=O)NCc1ccc2c(c1)CN(C1CCC(=O)NC1=O)C2=O)c1ccc(C)c(Cl)c1)C(=O)N[C@@H]1CN(c2nc(Nc3cn(C)nc3OC)c3ncn(C)c3n2)C[C@H]1F] |
| I-42 | 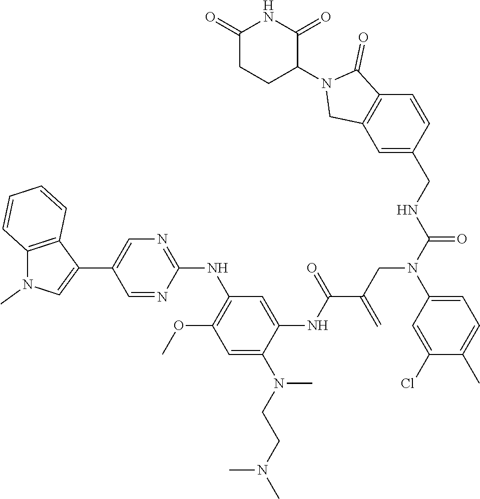 |
| I-43 | 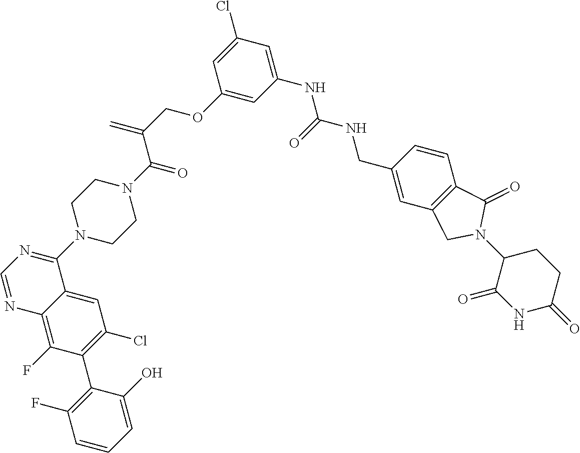 [SMILES: C=C(COc1cc(Cl)cc(NC(=O)NCc2ccc3c(c2)CN(C2CCC(=O)NC2=O)C3=O)c1)C(=O)N1CCN(c2ncnc3c(F)c(-c4c(O)cccc4F)c(Cl)cc23)CC1] |
| I-44 | 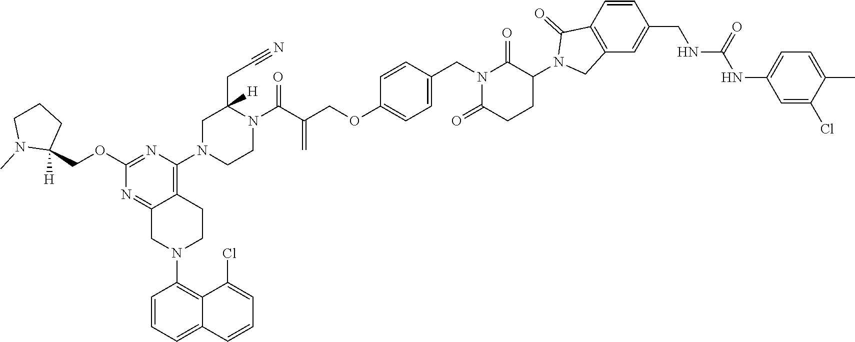 |
| I-45 | 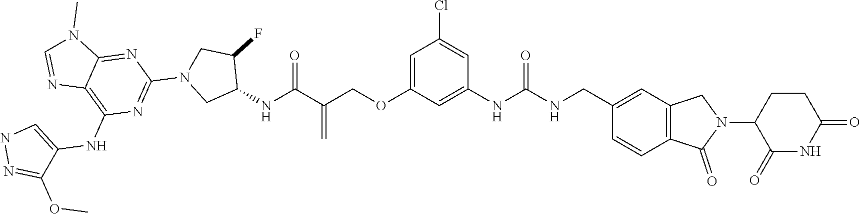 [SMILES: C=C(COc1cc(Cl)cc(NC(=O)NCc2ccc3c(c2)CN(C2CCC(=O)NC2=O)C3=O)c1)C(=O)N[C@@H]1CN(c2nc(Nc3c[nH]nc3OC)c3ncn(C)c3n2)C[C@H]1F] |
| I-46 | 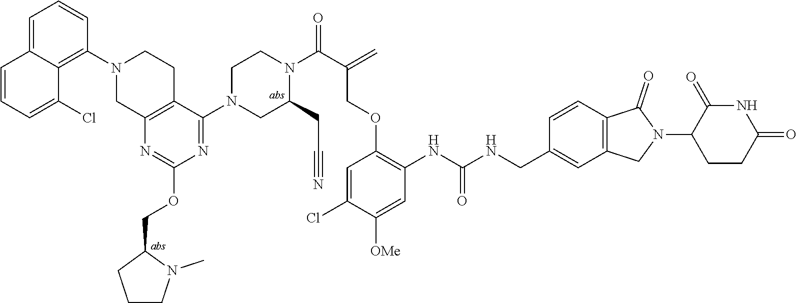 [SMILES: C=C(COc1cc(Cl)c(OC)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-47 | 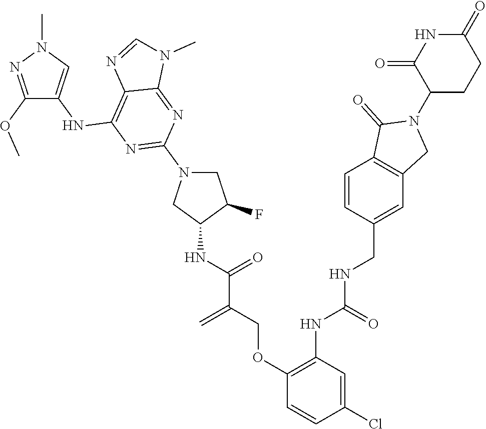 [SMILES: C=C(COc1ccc(Cl)cc1NC(=O)NCc1ccc2c(c1)CN(C1CCC(=O)NC1=O)C2=O)C(=O)N[C@@H]1CN(c2nc(Nc3cn(C)nc3OC)c3ncn(C)c3n2)C[C@H]1F] |
| I-48 | 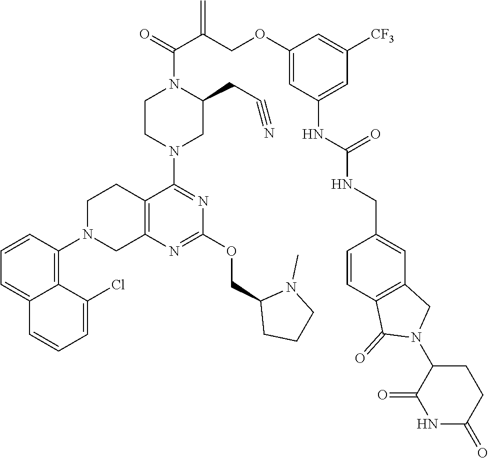 [SMILES: C=C(COc1cc(NC(=O)NCc2ccc3c(c2)CN(C2CCC(=O)NC2=O)C3=O)cc(C(F)(F)F)c1)C(=O)N1CCN(c2nc(OC[C@@H]3CCCN3C)nc3c2CCN(c2cccc4cccc(Cl)c24)C3)C[C@@H]1CC#N] |
| I-49 | 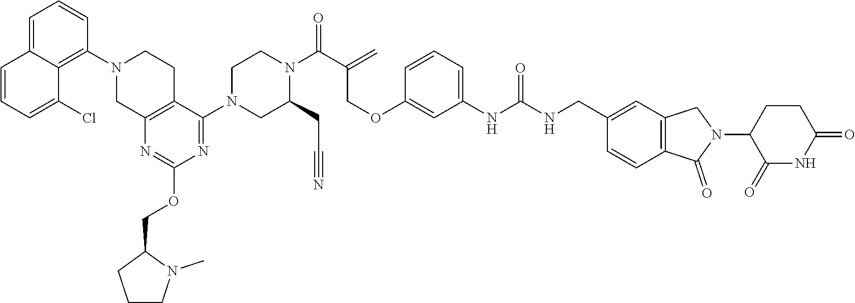 [SMILES: C=C(COc1cccc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-50 | 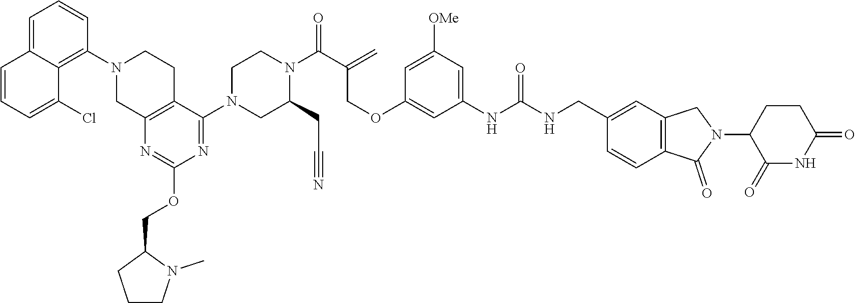 [SMILES: C=C(COc1cc(NC(=O)NCc2ccc3c(c2)CN(C2CCC(=O)NC2=O)C3=O)cc(OC)c1)C(=O)N1CCN(c2nc(OC[C@@H]3CCCN3C)nc3c2CCN(c2cccc4cccc(Cl)c24)C3)C[C@@H]1CC#N] |
| I-51 | 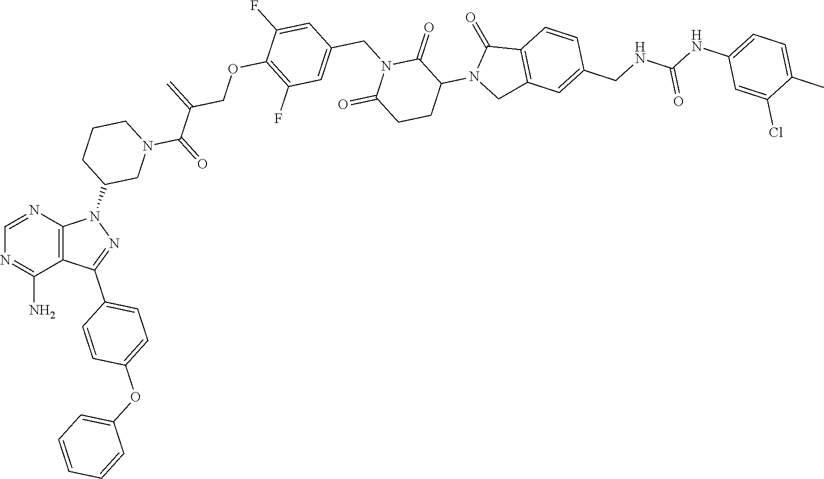 |
| I-52 | 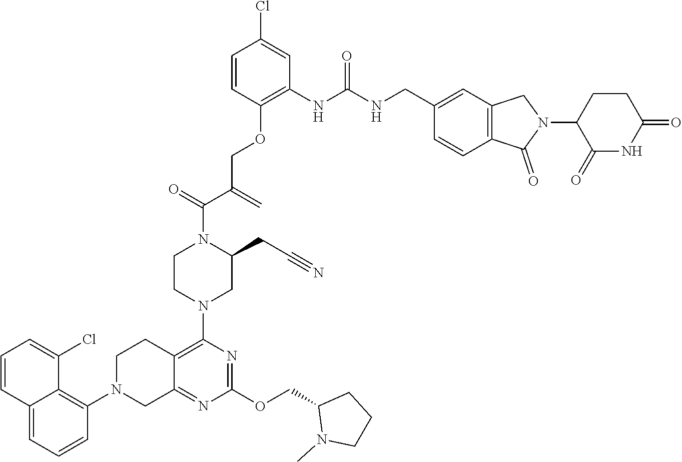 [SMILES: C=C(COc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-53 | 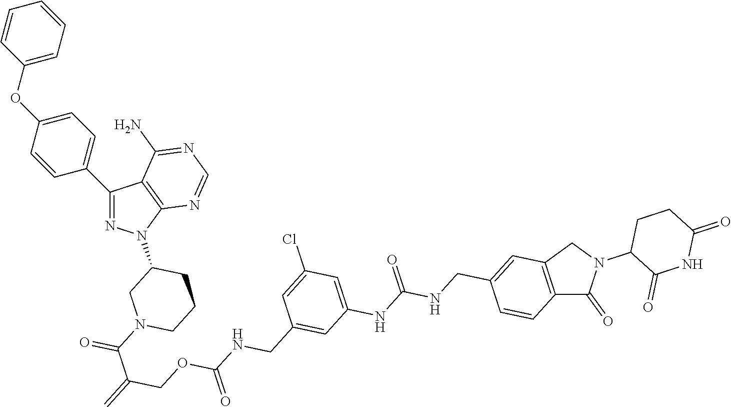 |
| I-54 | 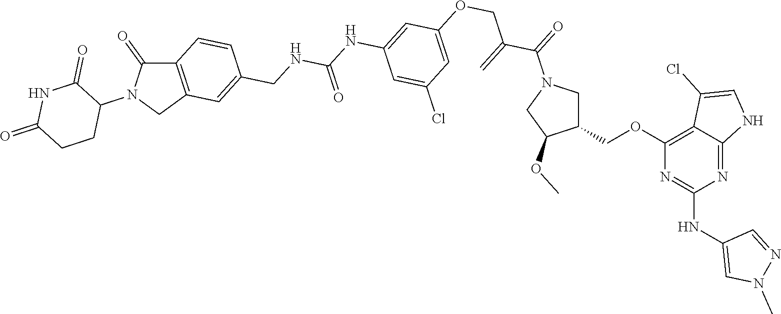 [SMILES: C=C(COc1cc(Cl)cc(NC(=O)NCc2ccc3c(c2)CN(C2CCC(=O)NC2=O)C3=O)c1)C(=O)N1C[C@H](COc2nc(Nc3cnn(C)c3)nc3[nH]cc(Cl)c23)[C@@H](OC)C1] |
| I-55 | 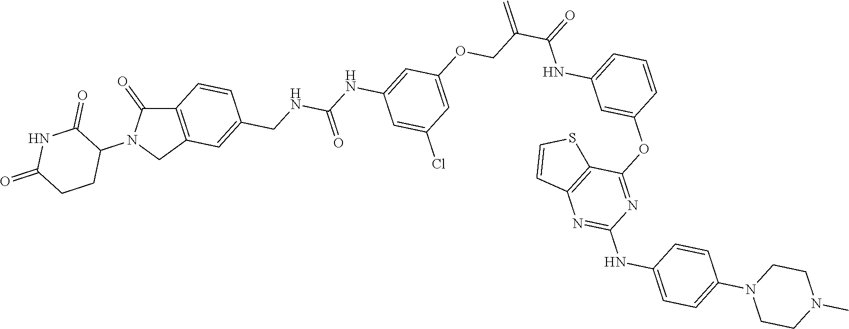 [SMILES: C=C(COc1cc(Cl)cc(NC(=O)NCc2ccc3c(c2)CN(C2CCC(=O)NC2=O)C3=O)c1)C(=O)Nc1cccc(Oc2nc(Nc3ccc(N4CCN(C)CC4)cc3)nc3ccsc23)c1] |
| I-56 | 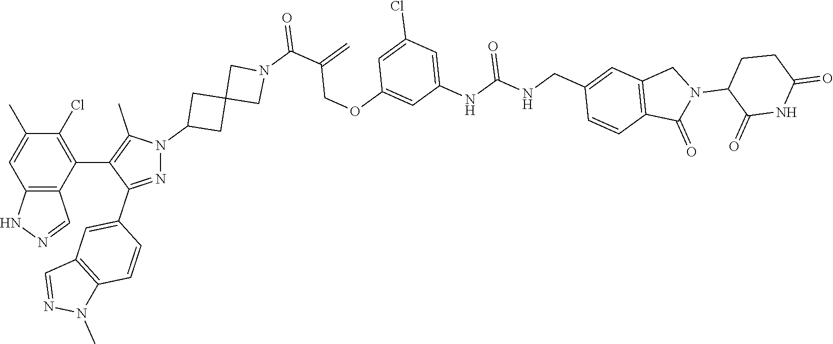 [SMILES: C=C(COc1cc(Cl)cc(NC(=O)NCc2ccc3c(c2)CN(C2CCC(=O)NC2=O)C3=O)c1)C(=O)N1CC2(CC(n3nc(-c4ccc5c(cnn5C)c4)c(-c4c(Cl)c(C)cc5[nH]ncc45)c3C)C2)C1] |
| I-57 | 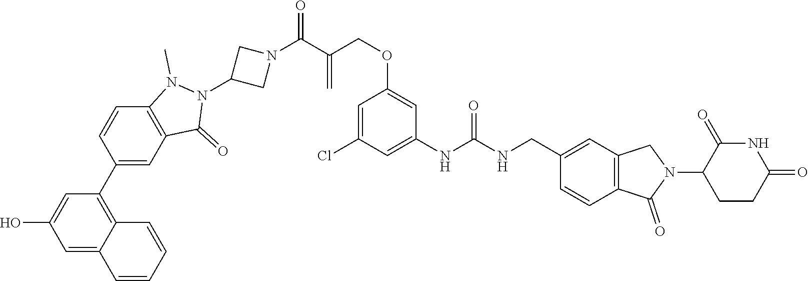 [SMILES: C=C(COc1cc(Cl)cc(NC(=O)NCc2ccc3c(c2)CN(C2CCC(=O)NC2=O)C3=O)c1)C(=O)N1CC(n2c(=O)c3cc(-c4cc(O)cc5ccccc45)ccc3n2C)C1] |
| I-58 | 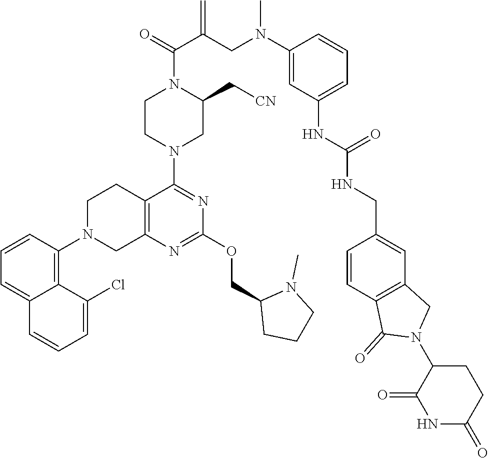 [SMILES: C=C(CN(C)c1cccc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-59 | 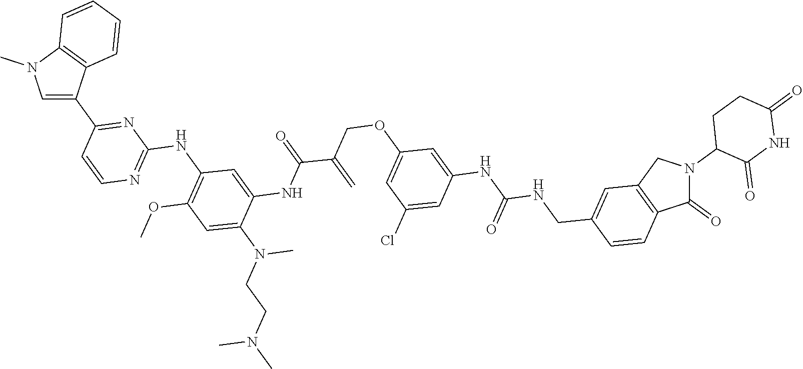 [SMILES: C=C(COc1cc(Cl)cc(NC(=O)NCc2ccc3c(c2)CN(C2CCC(=O)NC2=O)C3=O)c1)C(=O)Nc1cc(Nc2nccc(-c3cn(C)c4ccccc34)n2)c(OC)cc1N(C)CCN(C)C] |
| I-60 | 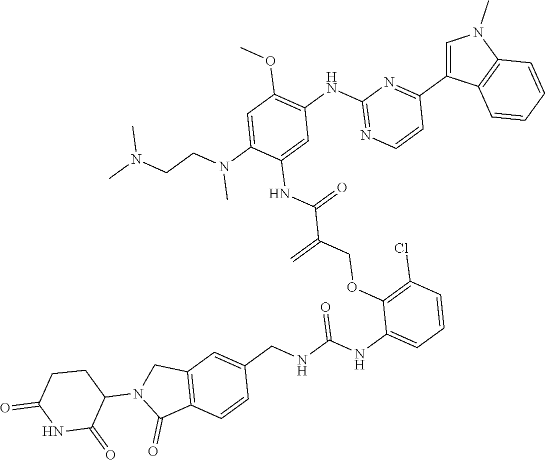 [SMILES: C=C(COc1c(Cl)cccc1NC(=O)NCc1ccc2c(c1)CN(C1CCC(=O)NC1=O)C2=O)C(=O)Nc1cc(Nc2nccc(-c3cn(C)c4ccccc34)n2)c(OC)cc1N(C)CCN(C)C] |
| I-61 | 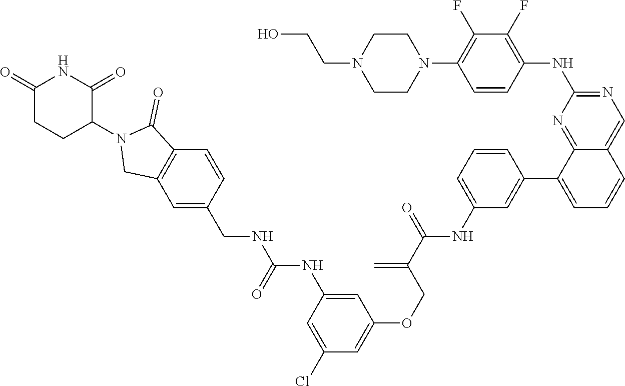 [SMILES: C=C(COc1cc(Cl)cc(NC(=O)NCc2ccc3c(c2)CN(C2CCC(=O)NC2=O)C3=O)c1)C(=O)Nc1cccc(-c2cccc3cnc(Nc4ccc(N5CCN(CCO)CC5)c(F)c4F)nc23)c1] |
| I-62 | 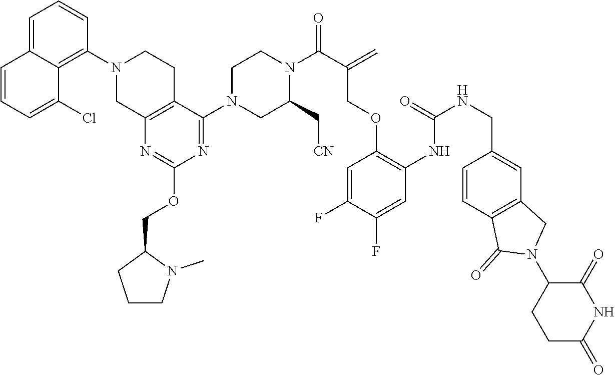 [SMILES: C=C(COc1cc(F)c(F)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-63 | 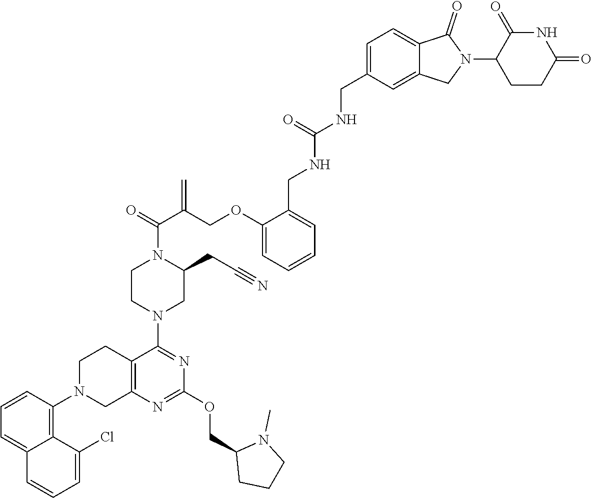 [SMILES: C=C(COc1ccccc1CNC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-64 | 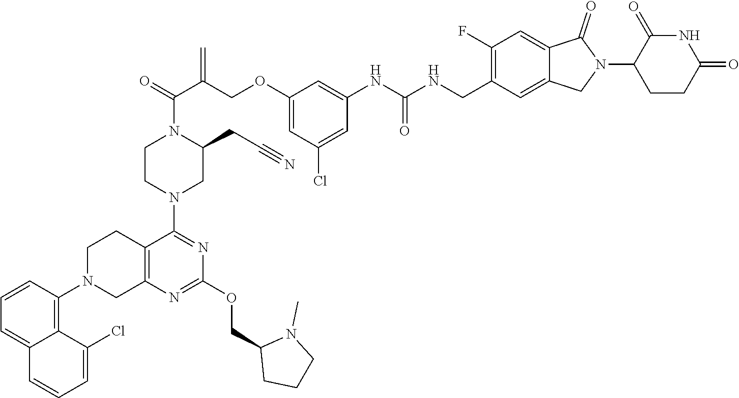 [SMILES: C=C(COc1cc(Cl)cc(NC(=O)NCc2cc3c(cc2F)C(=O)N(C2CCC(=O)NC2=O)C3)c1)C(=O)N1CCN(c2nc(OC[C@@H]3CCCN3C)nc3c2CCN(c2cccc4cccc(Cl)c24)C3)C[C@@H]1CC#N] |
| I-65 | 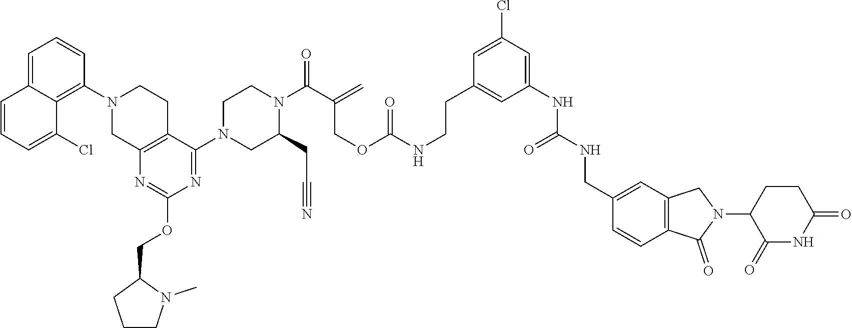 [SMILES: C=C(COC(=O)NCCc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-66 | 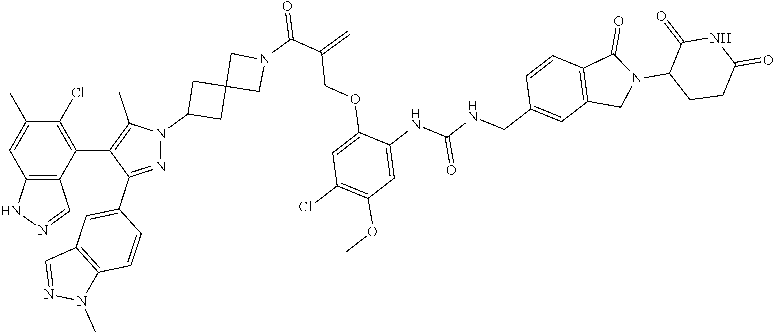 [SMILES: C=C(COc1cc(Cl)c(OC)cc1NC(=O)NCc1ccc2c(c1)CN(C1CCC(=O)NC1=O)C2=O)C(=O)N1CC2(CC(n3nc(-c4ccc5c(cnn5C)c4)c(-c4c(Cl)c(C)cc5[nH]ncc45)c3C)C2)C1] |
| I-67 | 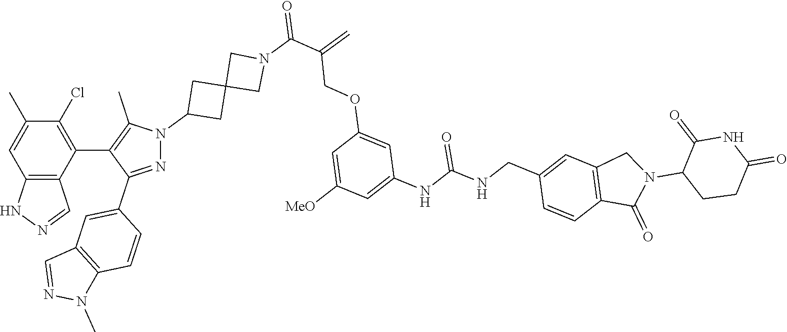 [SMILES: C=C(COc1cc(NC(=O)NCc2ccc3c(c2)CN(C2CCC(=O)NC2=O)C3=O)cc(OC)c1)C(=O)N1CC2(CC(n3nc(-c4ccc5c(cnn5C)c4)c(-c4c(Cl)c(C)cc5[nH]ncc45)c3C)C2)C1] |
| I-68 | 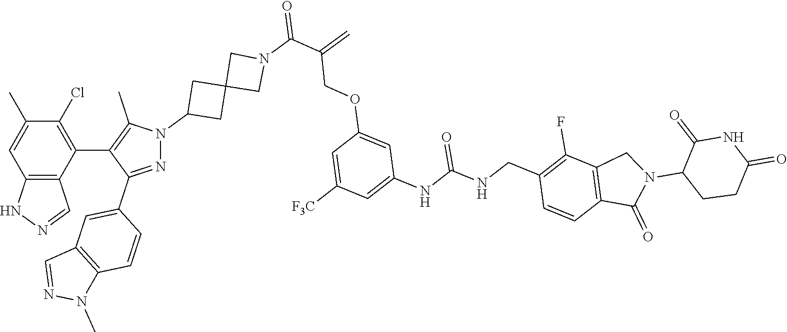 [SMILES: C=C(COc1cc(NC(=O)NCc2ccc3c(c2F)CN(C2CCC(=O)NC2=O)C3=O)cc(C(F)(F)F)c1)C(=O)N1CC2(CC(n3nc(-c4ccc5c(cnn5C)c4)c(-c4c(Cl)c(C)cc5[nH]ncc45)c3C)C2)C1] |
| I-69 | 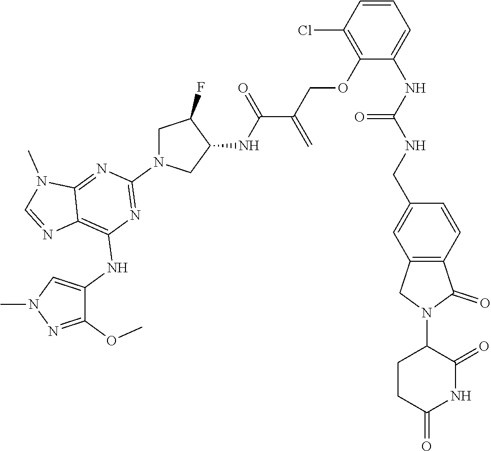 [SMILES: C=C(COc1c(Cl)cccc1NC(=O)NCc1ccc2c(c1)CN(C1CCC(=O)NC1=O)C2=O)C(=O)N[C@@H]1CN(c2nc(Nc3cn(C)nc3OC)c3ncn(C)c3n2)C[C@H]1F] |
| I-70 | 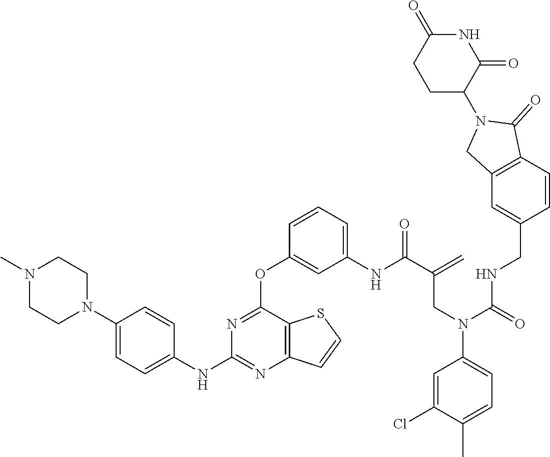 [SMILES: C=C(CN(C(=O)NCc1ccc2c(c1)CN(C1CCC(=O)NC1=O)C2=O)c1ccc(C)c(Cl)c1)C(=O)Nc1cccc(Oc2nc(Nc3ccc(N4CCN(C)CC4)cc3)nc3ccsc23)c1] |
| I-71 | 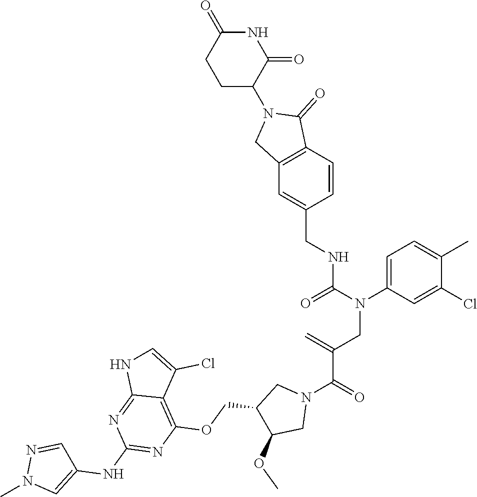 [SMILES: C=C(CN(C(=O)NCc1ccc2c(c1)CN(C1CCC(=O)NC1=O)C2=O)c1ccc(C)c(Cl)c1)C(=O)N1C[C@@H](COc2nc(Nc3cnn(C)c3)nc3[nH]cc(Cl)c23)[C@H](OC)C1] |
| I-72 | 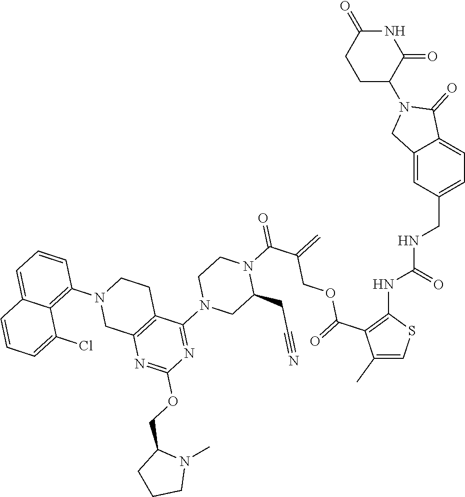 [SMILES: C=C(COC(=O)c1c(C)csc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-73 | 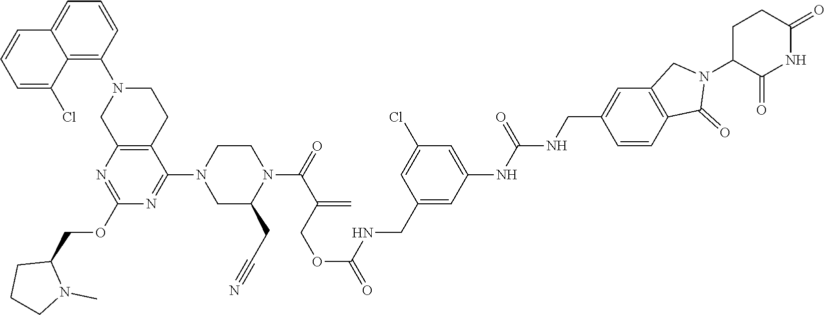 [SMILES: C=C(COC(=O)NCc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-74 | 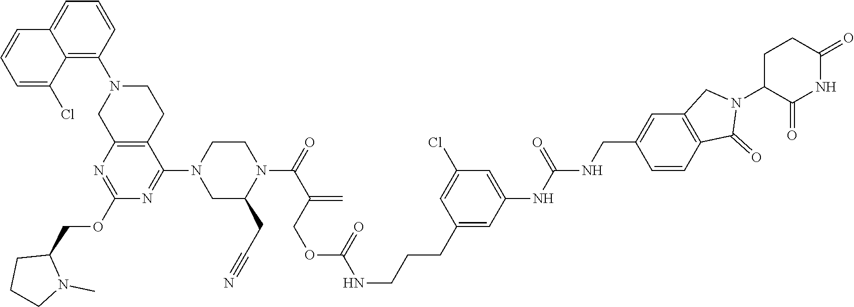 [SMILES: C=C(COC(=O)NCCCc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-75 | 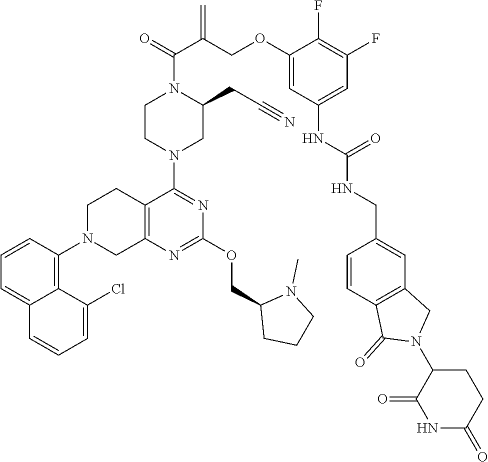 [SMILES: C=C(COc1cc(NC(=O)NCc2ccc3c(c2)CN(C2CCC(=O)NC2=O)C3=O)cc(F)c1F)C(=O)N1CCN(c2nc(OC[C@@H]3CCCN3C)nc3c2CCN(c2cccc4cccc(Cl)c24)C3)C[C@@H]1CC#N] |
| I-76 | 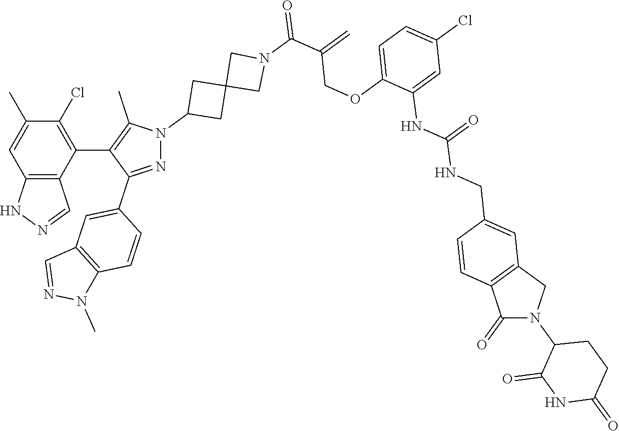 [SMILES: C=C(COc1ccc(Cl)cc1NC(=O)NCc1ccc2c(c1)CN(C1CCC(=O)NC1=O)C2=O)C(=O)N1CC2(CC(n3nc(-c4ccc5c(cnn5C)c4)c(-c4c(Cl)c(C)cc5[nH]ncc45)c3C)C2)C1] |
| I-77 | 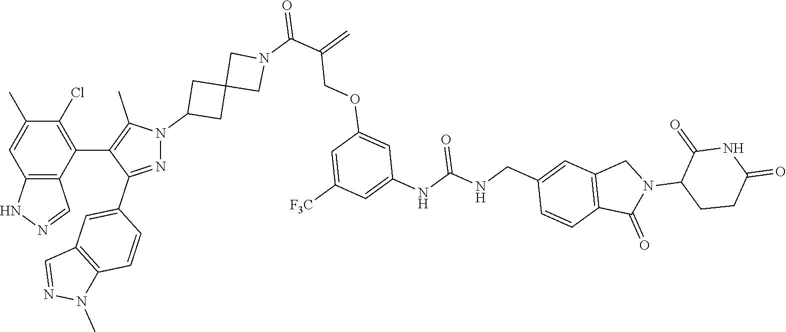 [SMILES: C=C(COc1cc(NC(=O)NCc2ccc3c(c2)CN(C2CCC(=O)NC2=O)C3=O)cc(C(F)(F)F)c1)C(=O)N1CC2(CC(n3nc(-c4ccc5c(cnn5C)c4)c(-c4c(Cl)c(C)cc5[nH]ncc45)c3C)C2)C1] |
| I-78 | 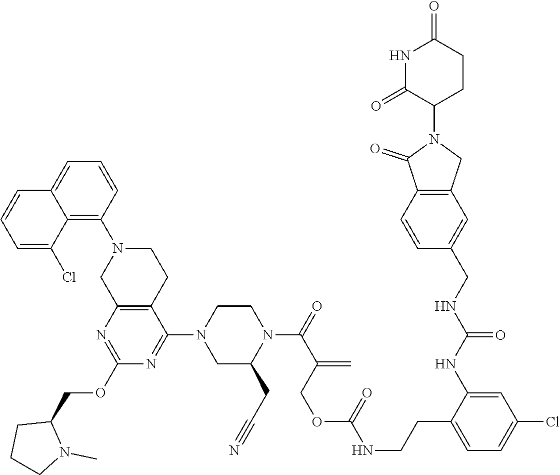 [SMILES: C=C(COC(=O)NCCc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-79 | 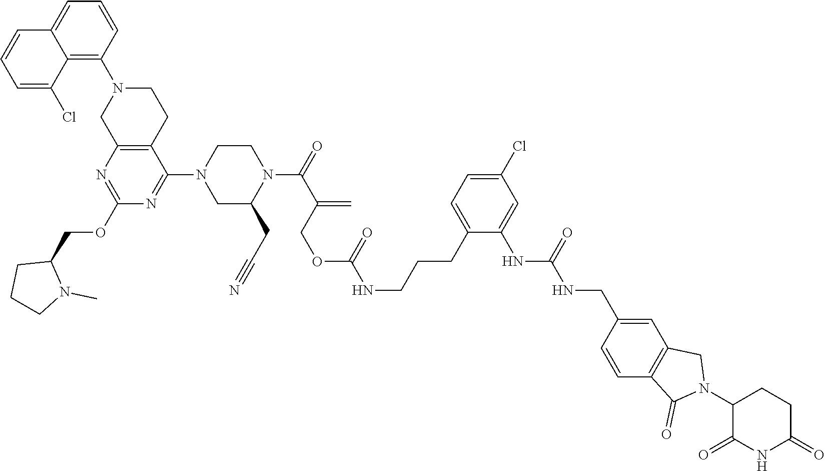 [SMILES: C=C(COC(=O)NCCCc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-80 | 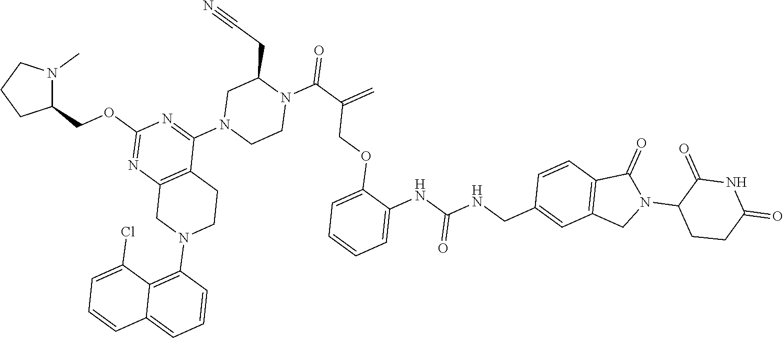 [SMILES: C=C(COc1ccccc1NC(=O)NCc1ccc2c(c1)CN(C1CCC(=O)NC1=O)C2=O)C(=O)N1CCN(c2nc(OC[C@H]3CCCN3C)nc3c2CCN(c2cccc4cccc(Cl)c24)C3)C[C@H]1CC#N] |
| I-81 | 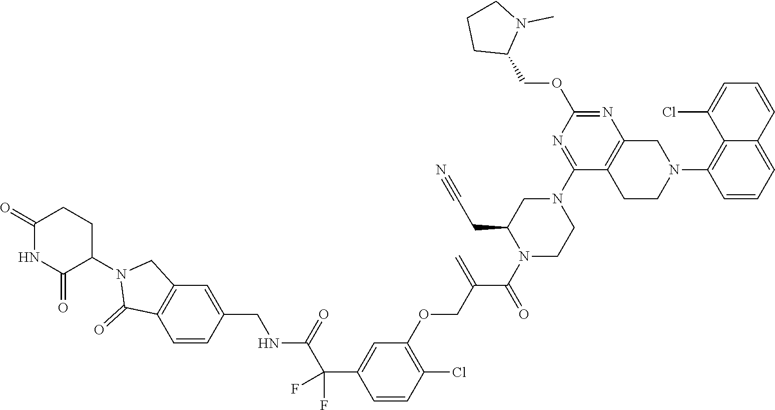 [SMILES: C=C(COc1cc(C(F)(F)C(=O)NCc2ccc3c(c2)CN(C2CCC(=O)NC2=O)C3=O)ccc1Cl)C(=O)N1CCN(c2nc(OC[C@@H]3CCCN3C)nc3c2CCN(c2cccc4cccc(Cl)c24)C3)C[C@@H]1CC#N] |
| I-82 | 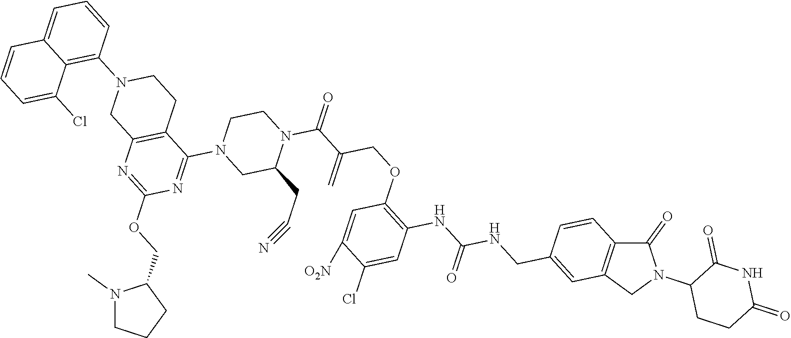 [SMILES: C=C(COc1cc([N+](=O)[O-])c(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-83 | 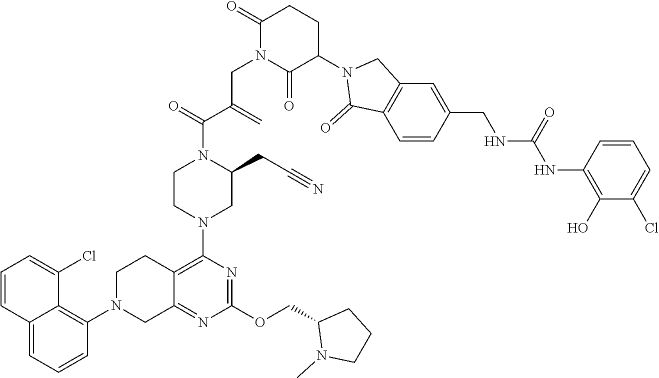 [SMILES: C=C(CN1C(=O)CCC(N2Cc3cc(CNC(=O)Nc4cccc(Cl)c4O)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-84 | 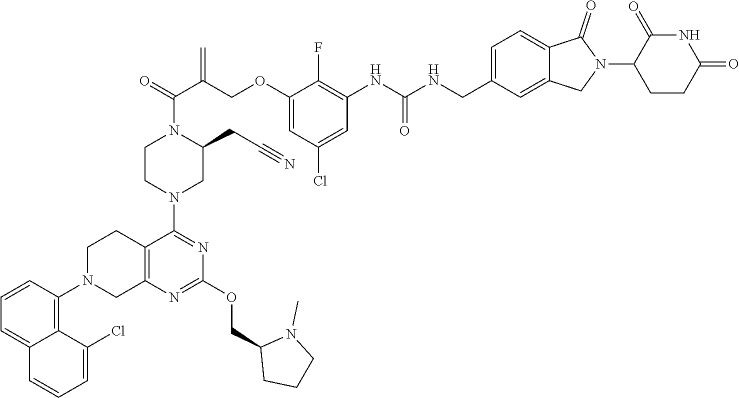 [SMILES: C=C(COc1cc(Cl)cc(NC(=O)NCc2ccc3c(c2)CN(C2CCC(=O)NC2=O)C3=O)c1F)C(=O)N1CCN(c2nc(OC[C@@H]3CCCN3C)nc3c2CCN(c2cccc4cccc(Cl)c24)C3)C[C@@H]1CC#N] |
| I-85 | 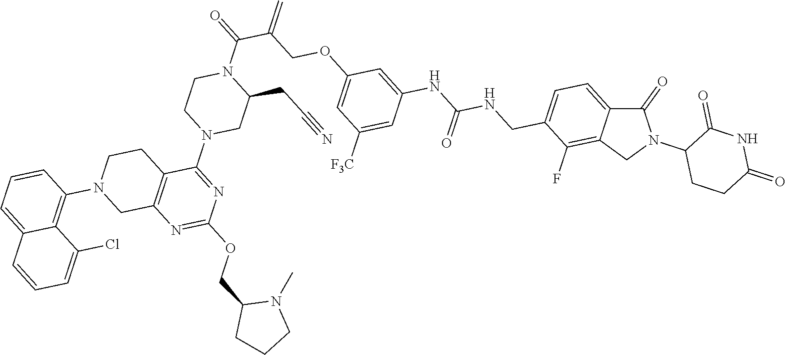 [SMILES: C=C(COc1cc(NC(=O)NCc2ccc3c(c2F)CN(C2CCC(=O)NC2=O)C3=O)cc(C(F)(F)F)c1)C(=O)N1CCN(c2nc(OC[C@@H]3CCCN3C)nc3c2CCN(c2cccc4cccc(Cl)c24)C3)C[C@@H]1CC#N] |
| I-86 | 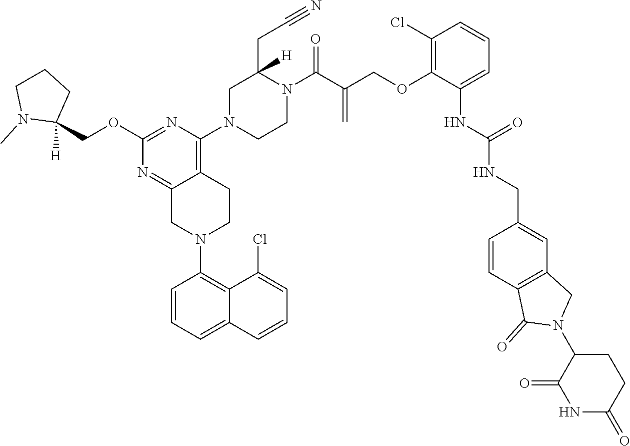 [SMILES: C=C(COc1c(Cl)cccc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-87 | 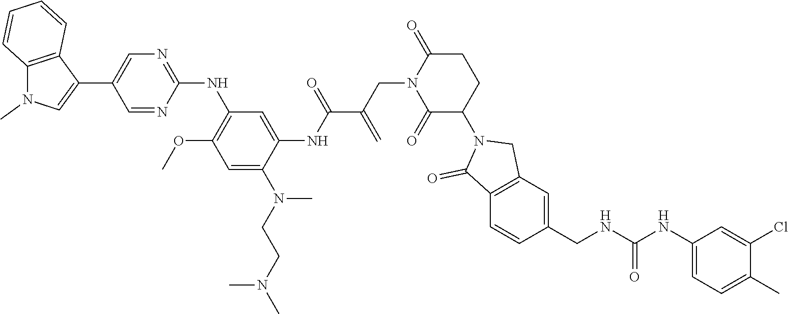 [SMILES: C=C(CN1C(=O)CCC(N2Cc3cc(CNC(=O)Nc4ccc(C)c(Cl)c4)ccc3C2=O)C1=O)C(=O)Nc1cc(Nc2ncc(-c3cn(C)c4ccccc34)cn2)c(OC)cc1N(C)CCN(C)C] |
| I-88 | 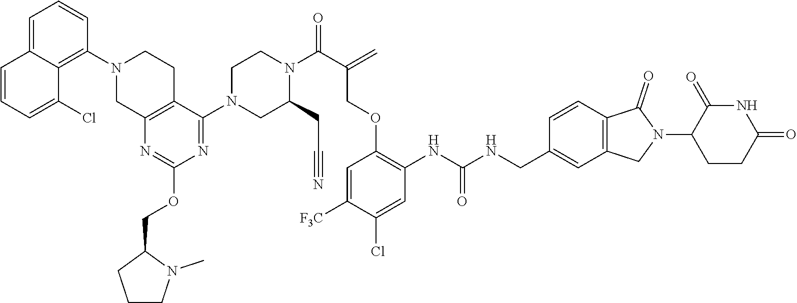 [SMILES: C=C(COc1cc(C(F)(F)F)c(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-89 | 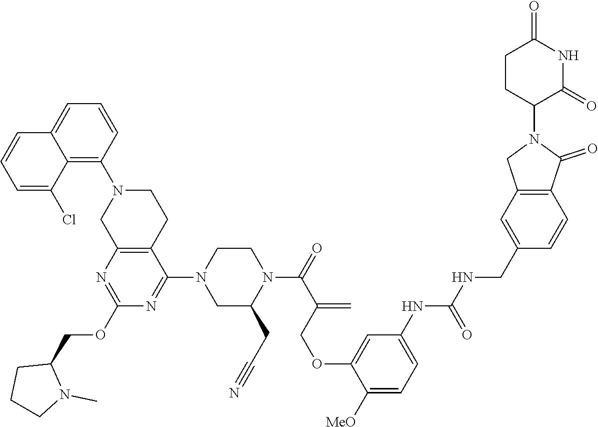 [SMILES: C=C(COc1cc(NC(=O)NCc2ccc3c(c2)CN(C2CCC(=O)NC2=O)C3=O)ccc1OC)C(=O)N1CCN(c2nc(OC[C@@H]3CCCN3C)nc3c2CCN(c2cccc4cccc(Cl)c24)C3)C[C@@H]1CC#N] |
| I-90 | 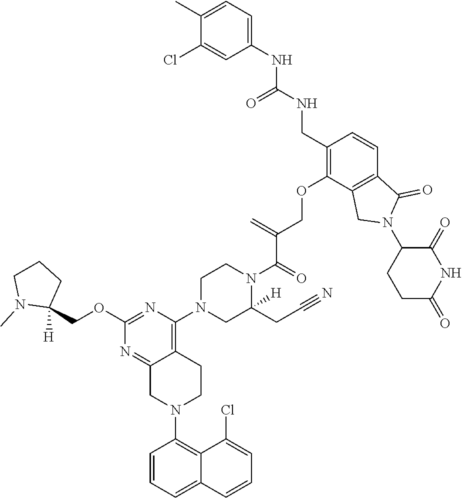 [SMILES: C=C(COc1c(CNC(=O)Nc2ccc(C)c(Cl)c2)ccc2c1CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-91 | 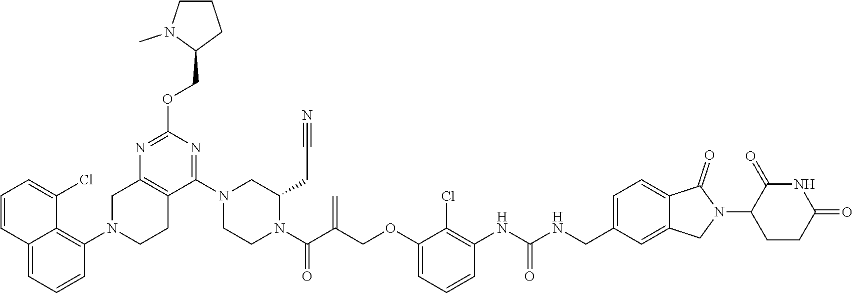 [SMILES: C=C(COc1cccc(NC(=O)NCc2ccc3c(c2)CN(C2CCC(=O)NC2=O)C3=O)c1Cl)C(=O)N1CCN(c2nc(OC[C@@H]3CCCN3C)nc3c2CCN(c2cccc4cccc(Cl)c24)C3)C[C@@H]1CC#N] |
| I-92 | 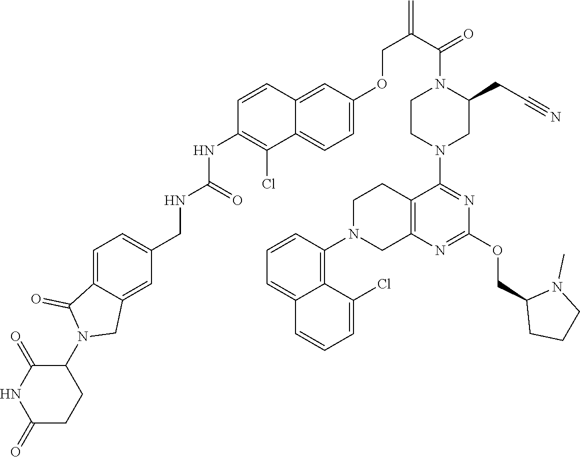 [SMILES: C=C(COc1ccc2c(Cl)c(NC(=O)NCc3ccc4c(c3)CN(C3CCC(=O)NC3=O)C4=O)ccc2c1)C(=O)N1CCN(c2nc(OC[C@@H]3CCCN3C)nc3c2CCN(c2cccc4cccc(Cl)c24)C3)C[C@@H]1CC#N] |
| I-93 | 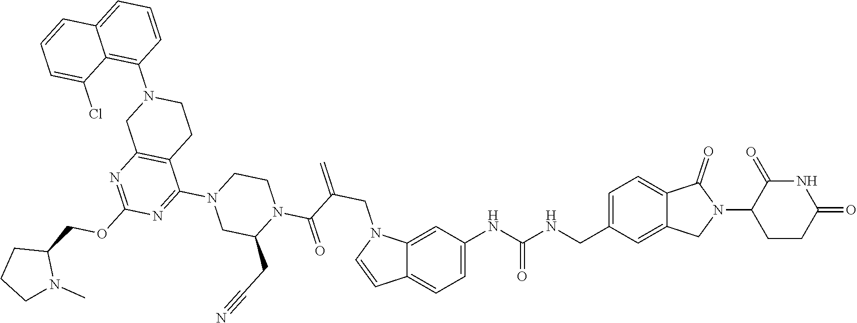 [SMILES: C=C(Cn1ccc2ccc(NC(=O)NCc3ccc4c(c3)CN(C3CCC(=O)NC3=O)C4=O)cc21)C(=O)N1CCN(c2nc(OC[C@@H]3CCCN3C)nc3c2CCN(c2cccc4cccc(Cl)c24)C3)C[C@@H]1CC#N] |
| I-94 | 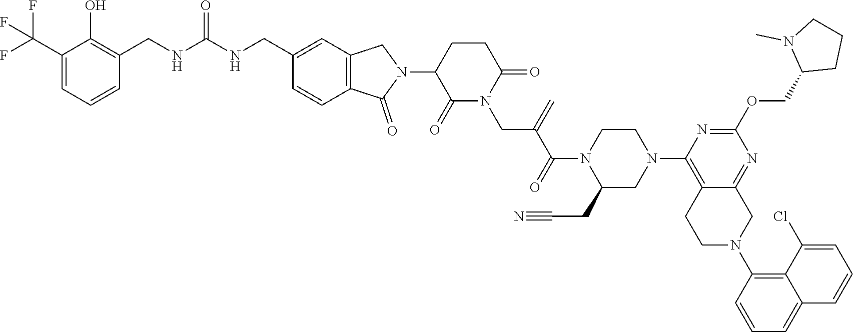 [SMILES: C=C(CN1C(=O)CCC(N2Cc3cc(CNC(=O)NCc4cccc(C(F)(F)F)c4O)ccc3C2=O)C1=O)C(=O)N1CCN(c2nc(OC[C@H]3CCCN3C)nc3c2CCN(c2cccc4cccc(Cl)c24)C3)C[C@H]1CC#N] |
| I-95 | 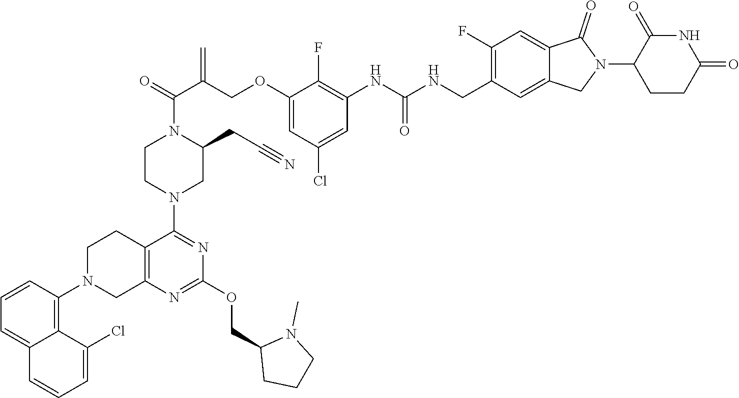 [SMILES: C=C(COc1cc(Cl)cc(NC(=O)NCc2cc3c(cc2F)C(=O)N(C2CCC(=O)NC2=O)C3)c1F)C(=O)N1CCN(c2nc(OC[C@@H]3CCCN3C)nc3c2CCN(c2cccc4cccc(Cl)c24)C3)C[C@@H]1CC#N] |
| I-96 | 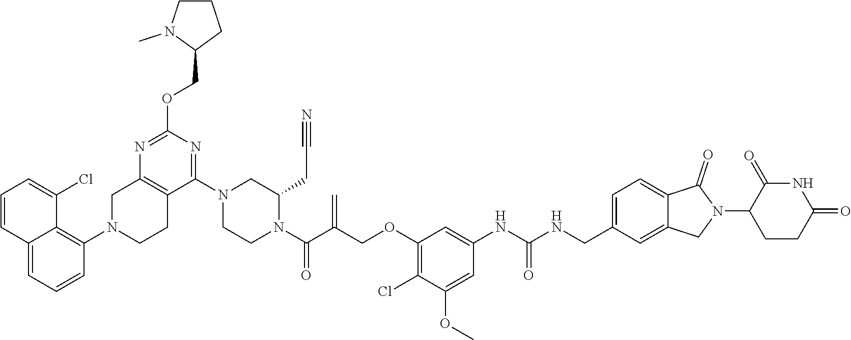 [SMILES: C=C(COc1cc(NC(=O)NCc2ccc3c(c2)CN(C2CCC(=O)NC2=O)C3=O)cc(OC)c1Cl)C(=O)N1CCN(c2nc(OC[C@@H]3CCCN3C)nc3c2CCN(c2cccc4cccc(Cl)c24)C3)C[C@@H]1CC#N] |
| I-97 | 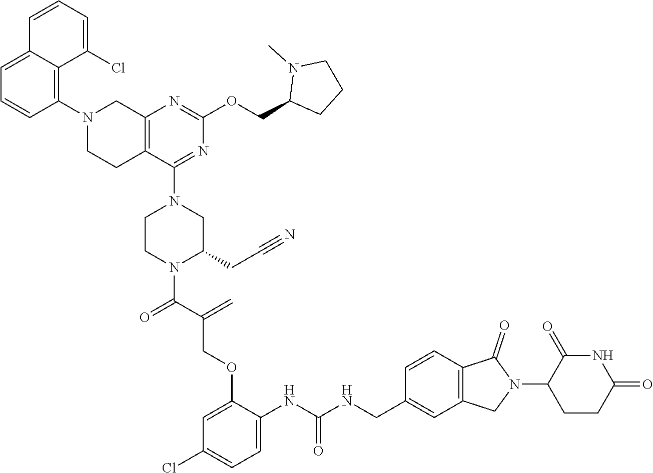 [SMILES: C=C(COc1cc(Cl)ccc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-98 | 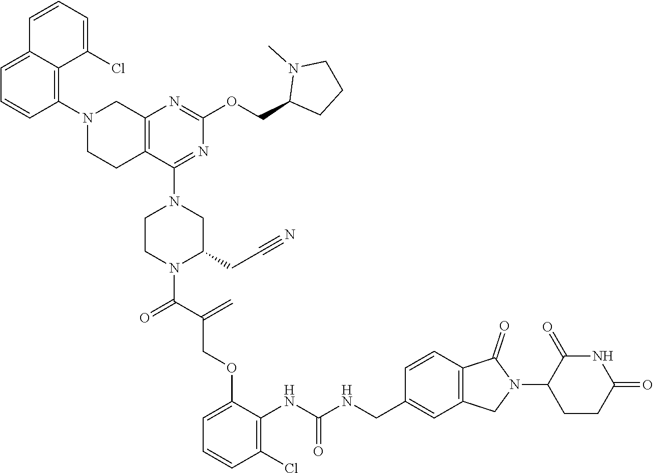 [SMILES: C=C(COc1cccc(Cl)c1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-99 | 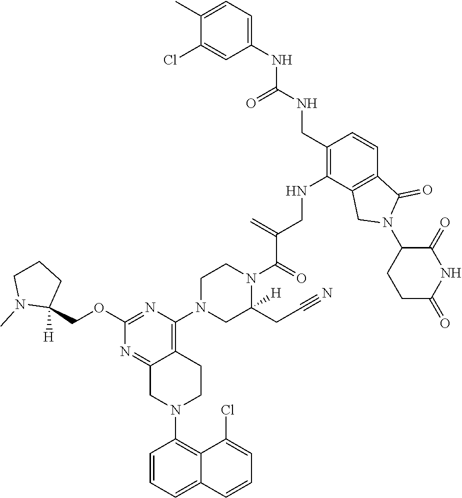 [SMILES: C=C(CNc1c(CNC(=O)Nc2ccc(C)c(Cl)c2)ccc2c1CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-100 | 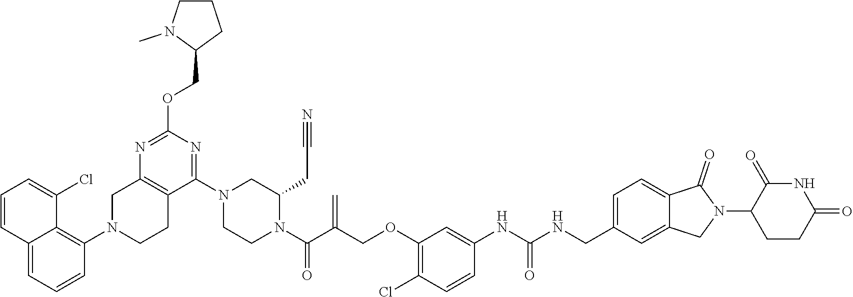 [SMILES: C=C(COc1cc(NC(=O)NCc2ccc3c(c2)CN(C2CCC(=O)NC2=O)C3=O)ccc1Cl)C(=O)N1CCN(c2nc(OC[C@@H]3CCCN3C)nc3c2CCN(c2cccc4cccc(Cl)c24)C3)C[C@@H]1CC#N] |
| I-101 | 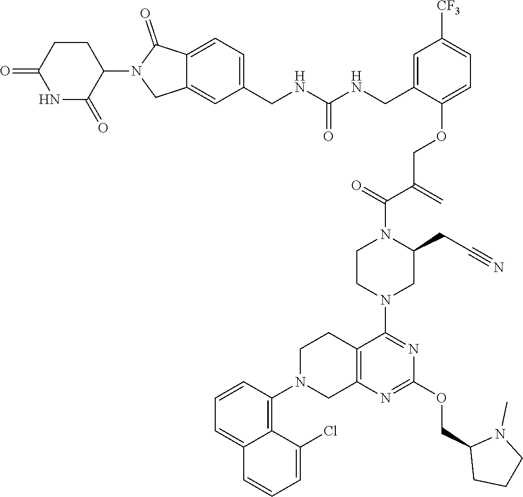 [SMILES: C=C(COc1ccc(C(F)(F)F)cc1CNC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-102 | 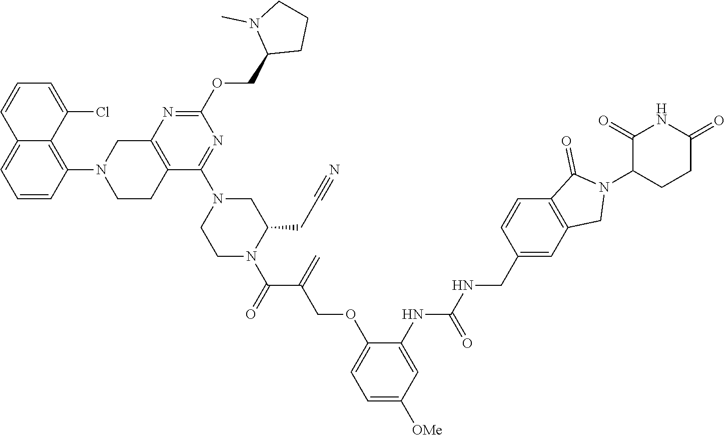 [SMILES: C=C(COc1ccc(OC)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-103 | 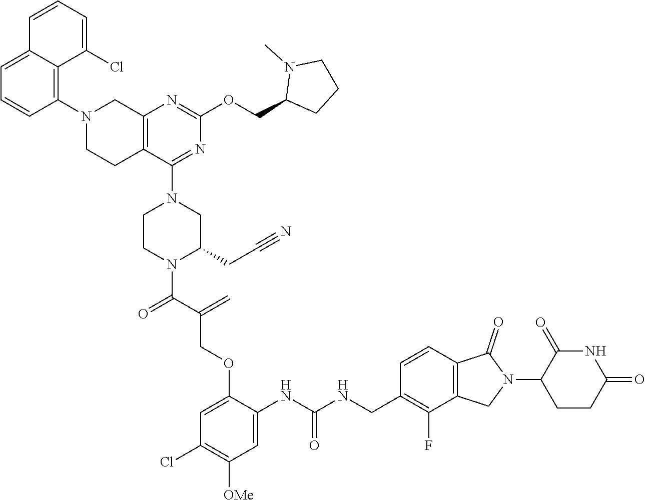 [SMILES: C=C(COc1cc(Cl)c(OC)cc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-104 | 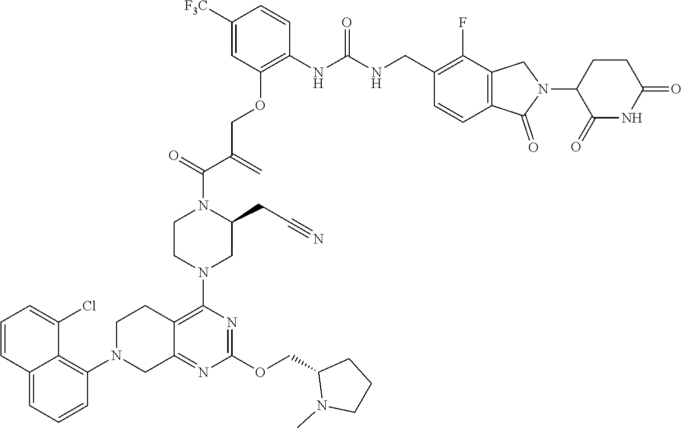 [SMILES: C=C(COc1cc(C(F)(F)F)ccc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-105 | 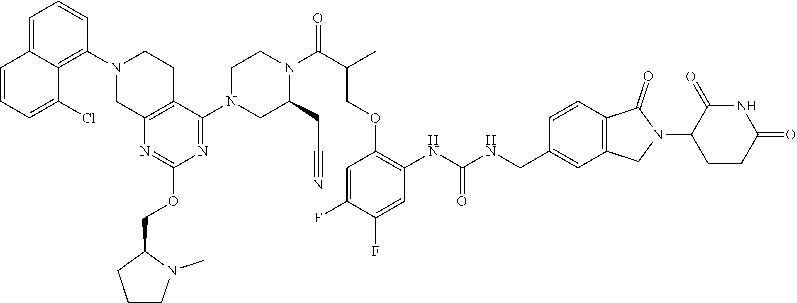 [SMILES: CC(COc1cc(F)c(F)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-106 | 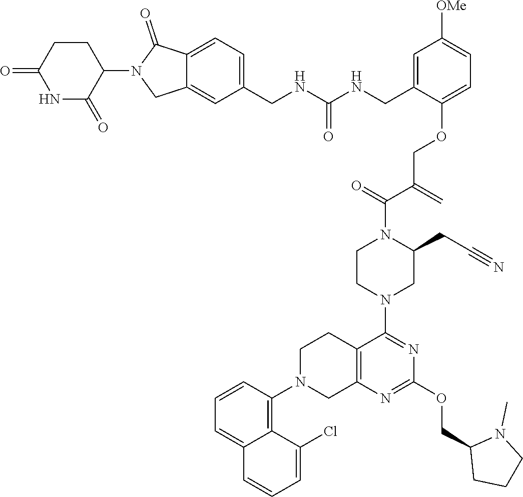 [SMILES: C=C(COc1ccc(OC)cc1CNC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-107 | 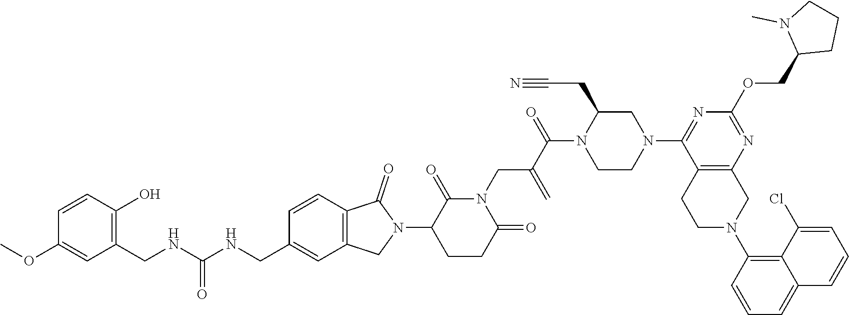 [SMILES: C=C(CN1C(=O)CCC(N2Cc3cc(CNC(=O)NCc4cc(OC)ccc4O)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-108 | 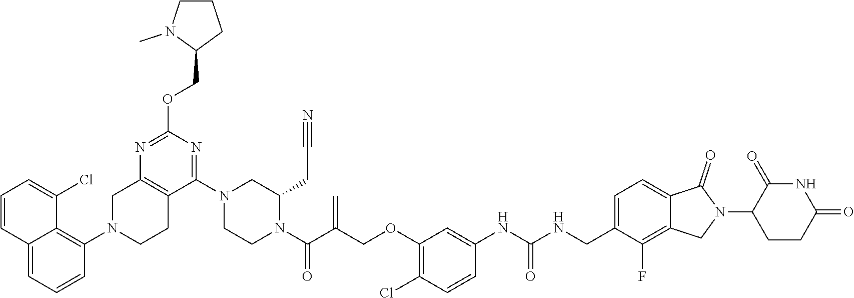 [SMILES: C=C(COc1cc(NC(=O)NCc2ccc3c(c2F)CN(C2CCC(=O)NC2=O)C3=O)ccc1Cl)C(=O)N1CCN(c2nc(OC[C@@H]3CCCN3C)nc3c2CCN(c2cccc4cccc(Cl)c24)C3)C[C@@H]1CC#N] |
| I-109 | 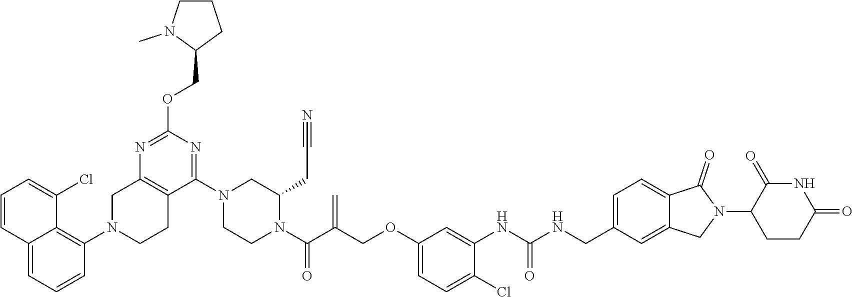 [SMILES: C=C(COc1ccc(Cl)c(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-110 | 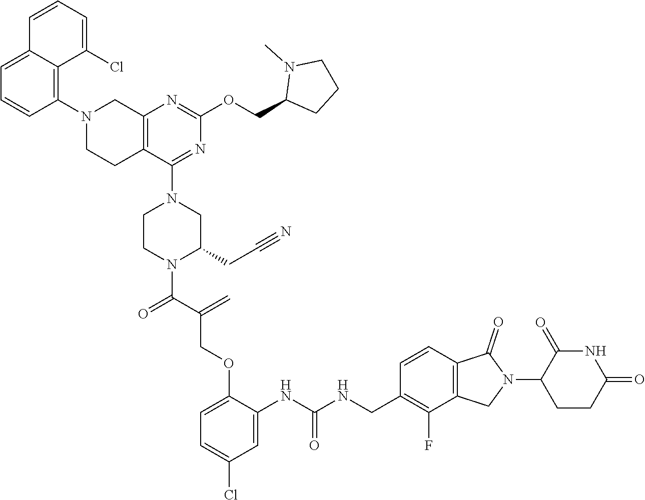 [SMILES: C=C(COc1ccc(Cl)cc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-111 | 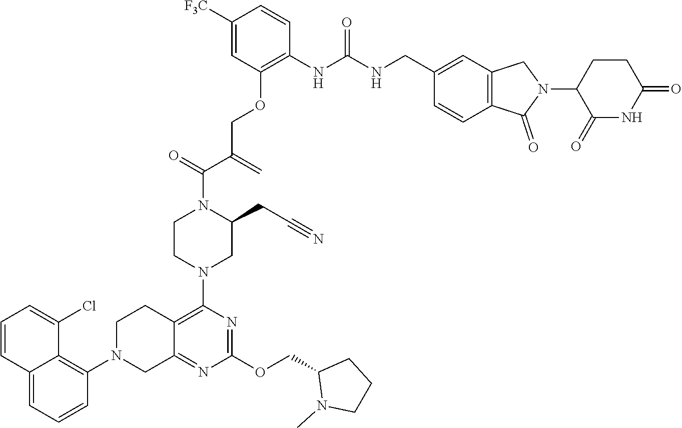 [SMILES: C=C(COc1cc(C(F)(F)F)ccc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-112 | 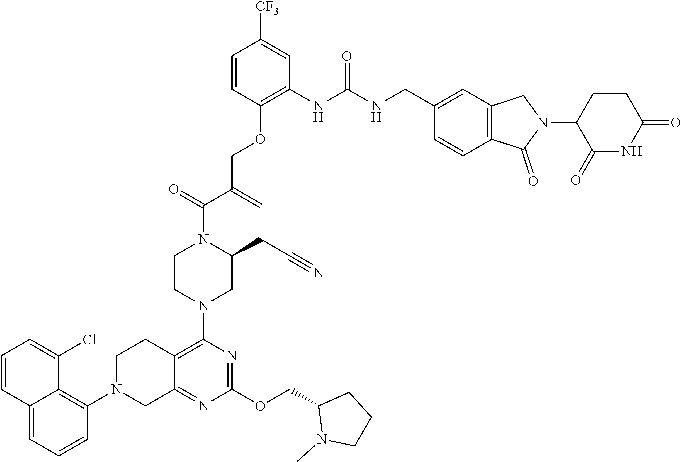 [SMILES: C=C(COc1ccc(C(F)(F)F)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-113 | 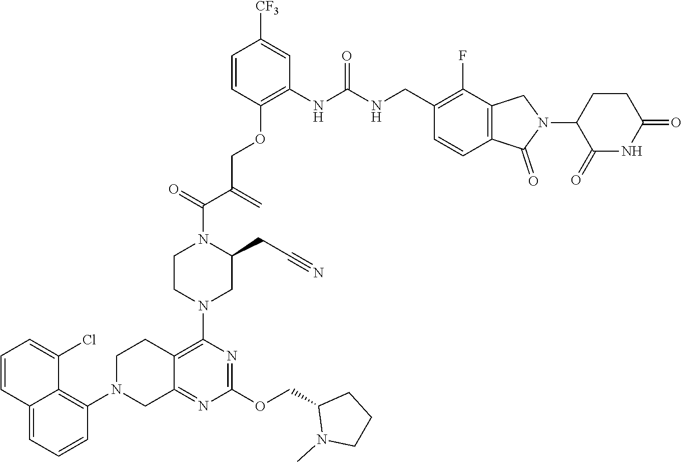 [SMILES: C=C(COc1ccc(C(F)(F)F)cc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-114 | 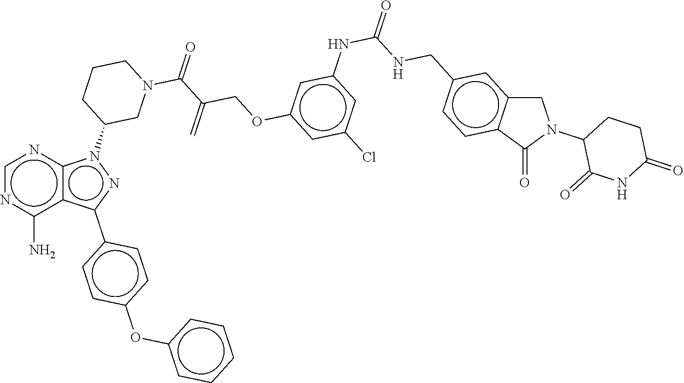 |
| I-115 | 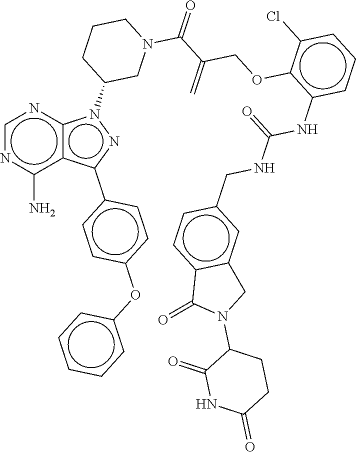 |
| I-116 | 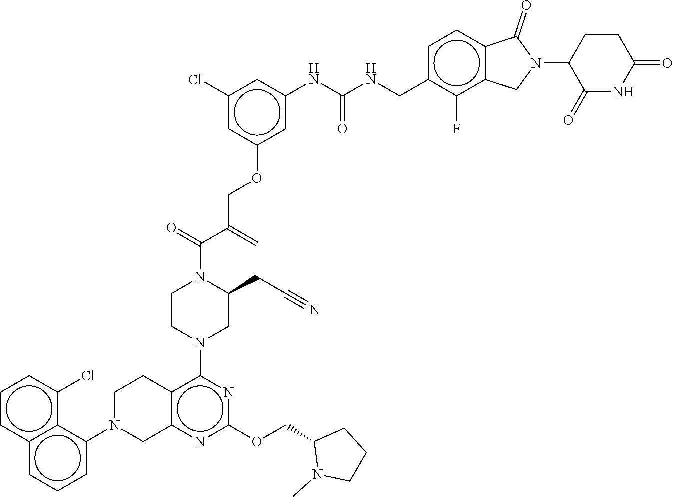 |

In certain embodiments, the compound is a compound in Table 2, or a pharmaceutically acceptable salt thereof. In certain embodiments, the compound is a compound in Table 2. In certain embodiments, the compound is a compound in Table 2-A, or a pharmaceutically acceptable salt thereof. In certain embodiments, the compound is a compound in Table 2-A.

TABLE 2
Compounds that Bind Cyclin K

| No. | Chemical Structure |
|---|---|
| II-1 | 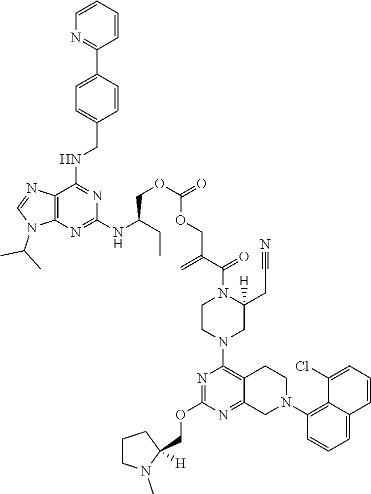 [SMILES: C=C(COC(=O)OC[C@@H](CC)Nc1nc(NCc2ccc(-c3ccccn3)cc2)c2ncn(C(C)C)c2n1)C(=O)N1CCN(c2nc(OC[C@@H]3CCCN3C)nc3c2CCN(c2cccc4cccc(Cl)c24)C3)C[C@@H]1CC#N] |
| II-2 | 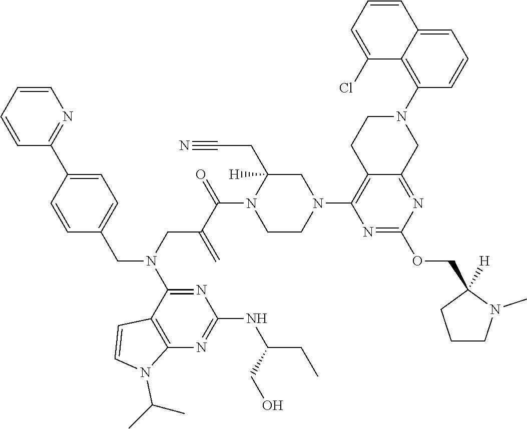 [SMILES: C=C(CN(Cc1ccc(-c2ccccn2)cc1)c1nc(N[C@H](CC)CO)nc2c1ccn2C(C)C)C(=O)N1CCN(c2nc(OC[C@@H]3CCCN3C)nc3c2CCN(c2cccc4cccc(Cl)c24)C3)C[C@@H]1CC#N] |
| II-3 | 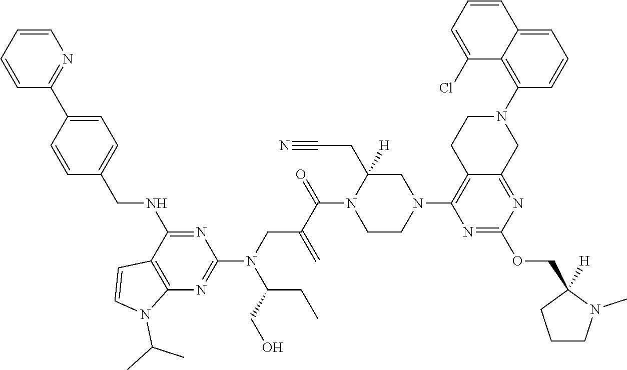 [SMILES: C=C(CN(c1nc(NCc2ccc(-c3ccccn3)cc2)c2ccn(C(C)C)c2n1)[C@H](CC)CO)C(=O)N1CCN(c2nc(OC[C@@H]3CCCN3C)nc3c2CCN(c2cccc4cccc(Cl)c24)C3)C[C@@H]1CC#N] |
| II-4 | 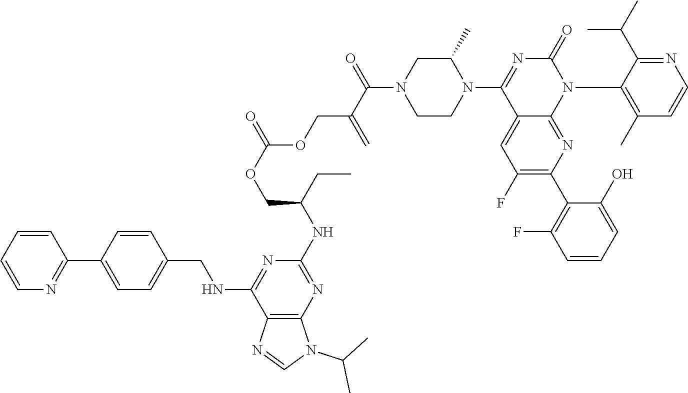 [SMILES: C=C(COC(=O)OC[C@@H](CC)Nc1nc(NCc2ccc(-c3ccccn3)cc2)c2ncn(C(C)C)c2n1)C(=O)N1CCN(c2nc(=O)n(-c3c(C)ccnc3C(C)C)c3nc(-c4c(O)cccc4F)c(F)cc23)[C@@H](C)C1] |
| II-5 | 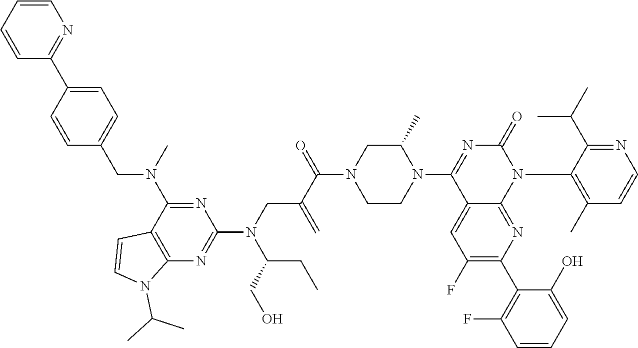 [SMILES: C=C(CN(c1nc(N(C)Cc2ccc(-c3ccccn3)cc2)c2ccn(C(C)C)c2n1)[C@H](CC)CO)C(=O)N1CCN(c2nc(=O)n(-c3c(C)ccnc3C(C)C)c3nc(-c4c(O)cccc4F)c(F)cc23)[C@@H](C)C1] |
| II-6 | 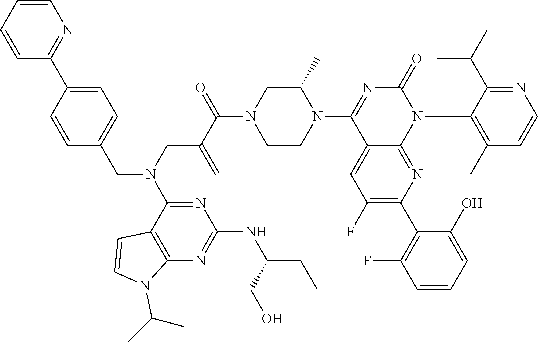 [SMILES: C=C(CN(Cc1ccc(-c2ccccn2)cc1)c1nc(N[C@H](CC)CO)nc2c1ccn2C(C)C)C(=O)N1CCN(c2nc(=O)n(-c3c(C)ccnc3C(C)C)c3nc(-c4c(O)cccc4F)c(F)cc23)[C@@H](C)C1] |
| II-7 | 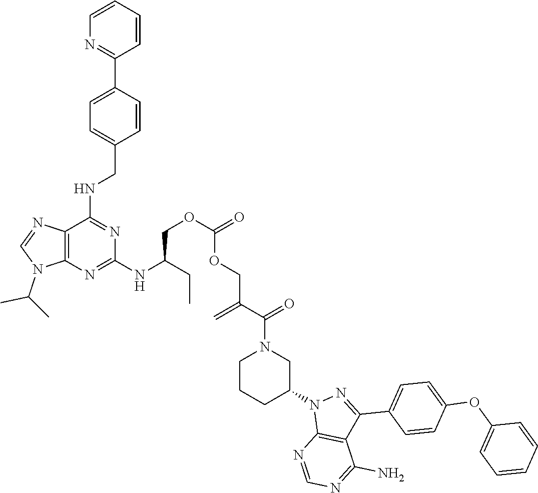 [SMILES: C=C(COC(=O)OC[C@@H](CC)Nc1nc(NCc2ccc(-c3ccccn3)cc2)c2ncn(C(C)C)c2n1)C(=O)N1CCC[C@@H](n2nc(-c3ccc(Oc4ccccc4)cc3)c3c(N)ncnc32)C1] |
| II-8 | 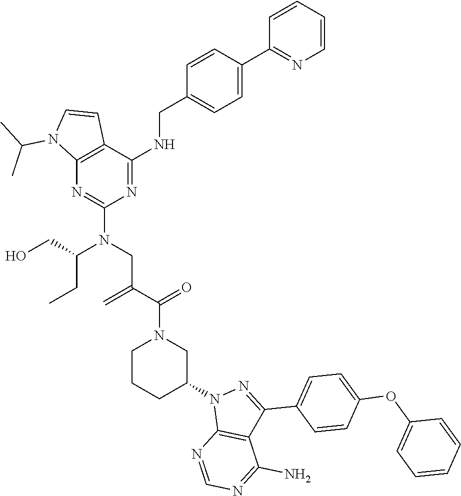 [SMILES: C=C(CN(c1nc(NCc2ccc(-c3ccccn3)cc2)c2ccn(C(C)C)c2n1)[C@H](CC)CO)C(=O)N1CCC[C@@H](n2nc(-c3ccc(Oc4ccccc4)cc3)c3c(N)ncnc32)C1] |
| II-9 | 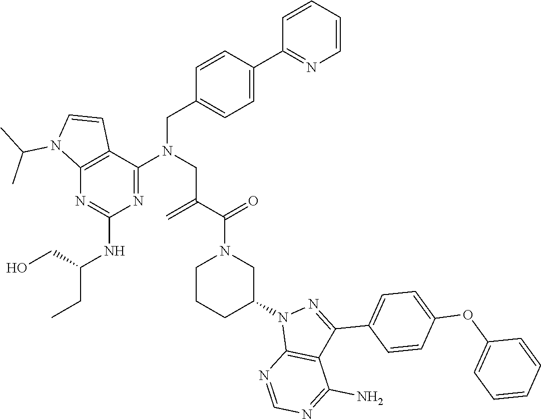 [SMILES: C=C(CN(Cc1ccc(-c2ccccn2)cc1)c1nc(N[C@H](CC)CO)nc2c1ccn2C(C)C)C(=O)N1CCC[C@@H](n2nc(-c3ccc(Oc4ccccc4)cc3)c3c(N)ncnc32)C1] |
| II-10 | 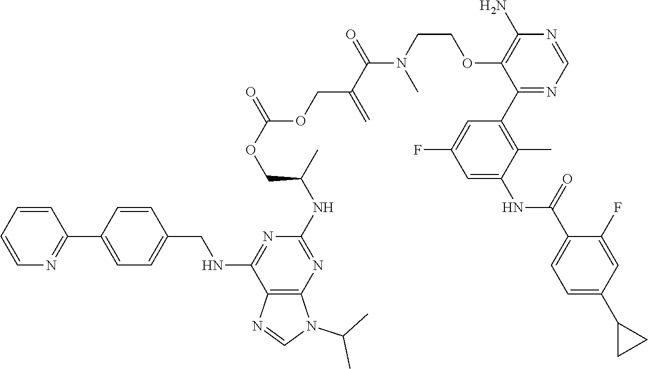 [SMILES: C=C(COC(=O)OC[C@@H](C)Nc1nc(NCc2ccc(-c3ccccn3)cc2)c2ncn(C(C)C)c2n1)C(=O)N(C)CCOc1c(N)ncnc1-c1cc(F)cc(NC(=O)c2ccc(C3CC3)cc2F)c1C] |
| II-11 | 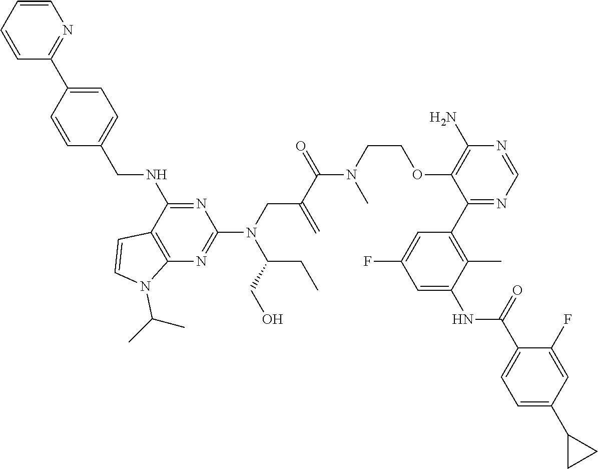 [SMILES: C=C(CN(c1nc(NCc2ccc(-c3ccccn3)cc2)c2ccn(C(C)C)c2n1)[C@H](CC)CO)C(=O)N(C)CCOc1c(N)ncnc1-c1cc(F)cc(NC(=O)c2ccc(C3CC3)cc2F)c1C] |
| II-12 | 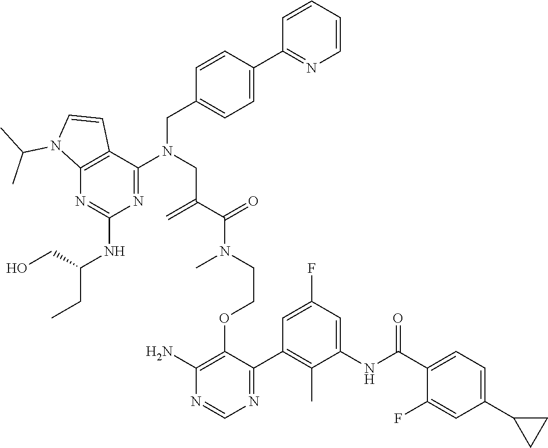 [SMILES: C=C(CN(Cc1ccc(-c2ccccn2)cc1)c1nc(N[C@H](CC)CO)nc2c1ccn2C(C)C)C(=O)N(C)CCOc1c(N)ncnc1-c1cc(F)cc(NC(=O)c2ccc(C3CC3)cc2F)c1C] |
| II-13 | 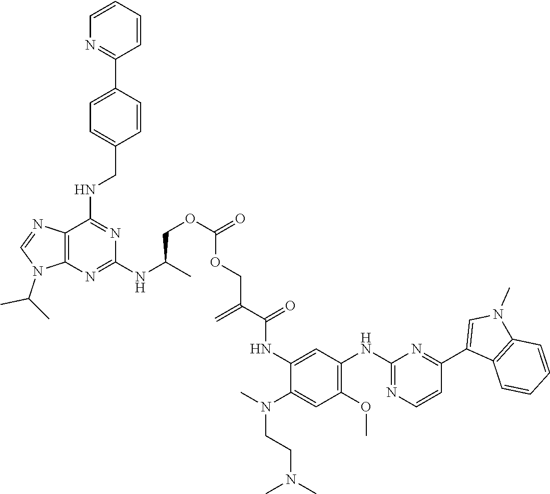 [SMILES: C=C(COC(=O)OC[C@@H](C)Nc1nc(NCc2ccc(-c3ccccn3)cc2)c2ncn(C(C)C)c2n1)C(=O)Nc1cc(Nc2nccc(-c3cn(C)c4ccccc34)n2)c(OC)cc1N(C)CCN(C)C] |
| II-14 | 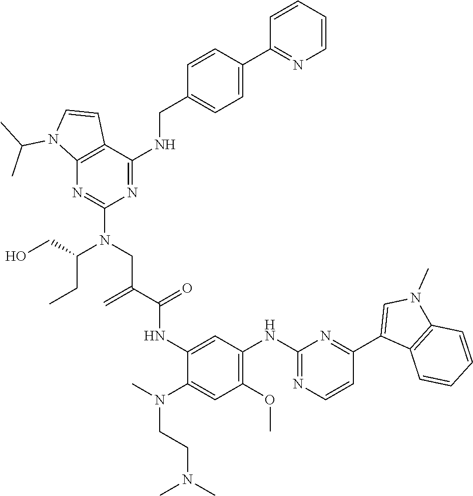 [SMILES: C=C(CN(c1nc(NCc2ccc(-c3ccccn3)cc2)c2ccn(C(C)C)c2n1)[C@H](CC)CO)C(=O)Nc1cc(Nc2nccc(-c3cn(C)c4ccccc34)n2)c(OC)cc1N(C)CCN(C)C] |
| II-15 | 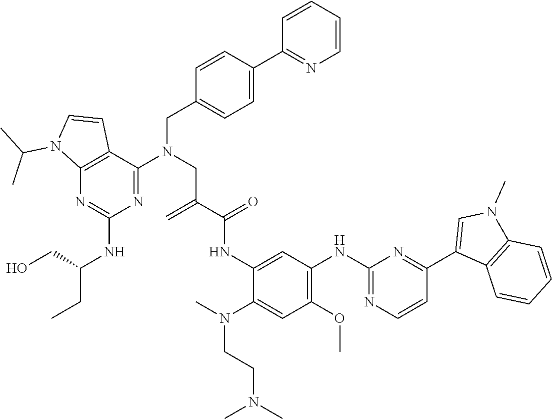 [SMILES: C=C(CN(Cc1ccc(-c2ccccn2)cc1)c1nc(N[C@H](CC)CO)nc2c1ccn2C(C)C)C(=O)Nc1cc(Nc2nccc(-c3cn(C)c4ccccc34)n2)c(OC)cc1N(C)CCN(C)C] |
| II-16 | 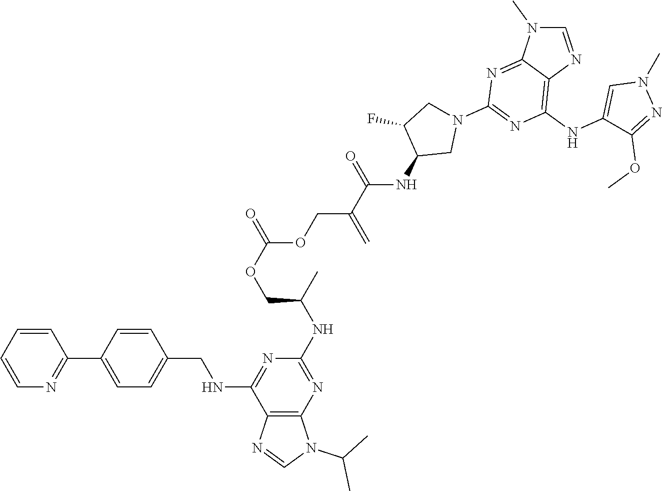 [SMILES: C=C(COC(=O)OC[C@@H](C)Nc1nc(NCc2ccc(-c3ccccn3)cc2)c2ncn(C(C)C)c2n1)C(=O)N[C@@H]1CN(c2nc(Nc3cn(C)nc3OC)c3ncn(C)c3n2)C[C@H]1F] |
| II-17 | 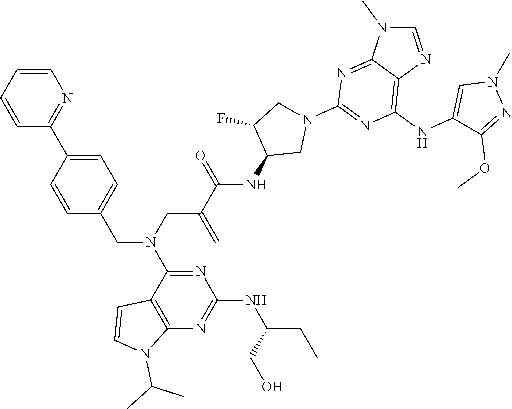 |
| II-18 | 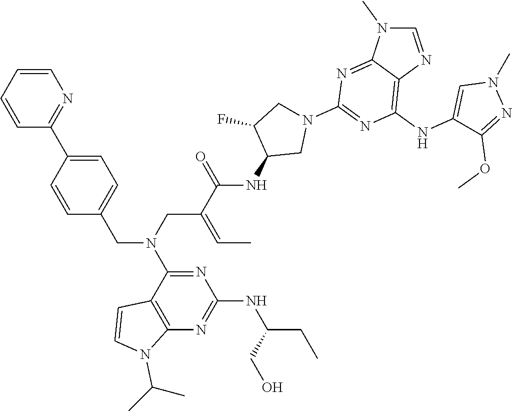 [SMILES: C/C=C(/CN(Cc1ccc(-c2ccccn2)cc1)c1nc(N[C@H](CC)CO)nc2c1ccn2C(C)C)C(=O)N[C@@H]1CN(c2nc(Nc3cn(C)nc3OC)c3ncn(C)c3n2)C[C@H]1F] |
| II-19 | 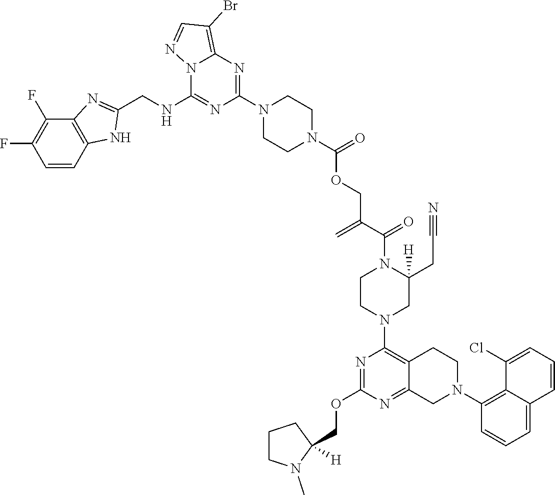 [SMILES: C=C(COC(=O)N1CCN(c2nc(NCc3nc4c(F)c(F)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| II-20 | 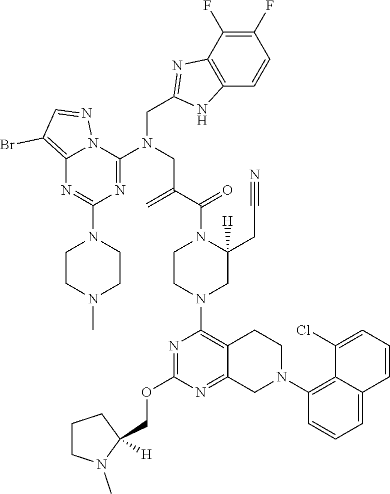 [SMILES: C=C(CN(Cc1nc2c(F)c(F)ccc2[nH]1)c1nc(N2CCN(C)CC2)nc2c(Br)cnn12)C(=O)N1CCN(c2nc(OC[C@@H]3CCCN3C)nc3c2CCN(c2cccc4cccc(Cl)c24)C3)C[C@@H]1CC#N] |
| II-21 | 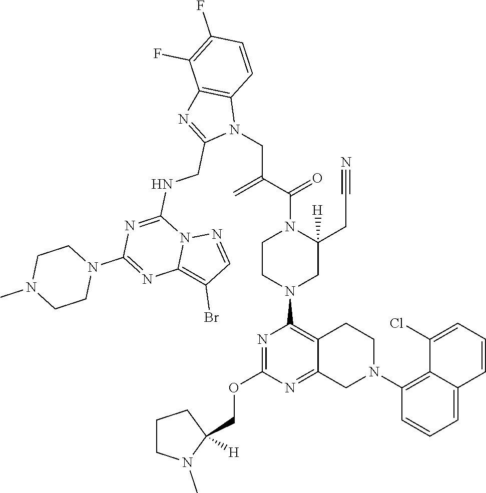 |
| II-22 | 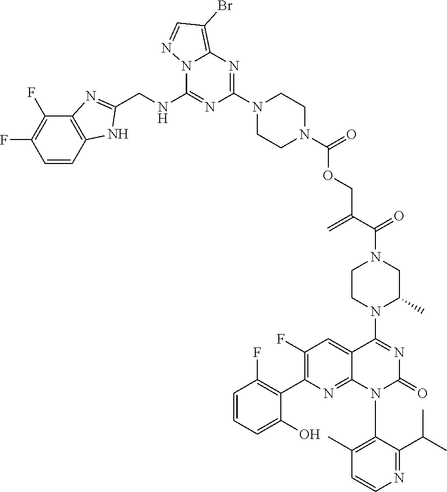 [SMILES: C=C(COC(=O)N1CCN(c2nc(NCc3nc4c(F)c(F)ccc4[nH]3)n3ncc(Br)c3n2)CC1)C(=O)N1CCN(c2nc(=O)n(-c3c(C)ccnc3C(C)C)c3nc(-c4c(O)cccc4F)c(F)cc23)[C@@H](C)C1] |
| II-23 | 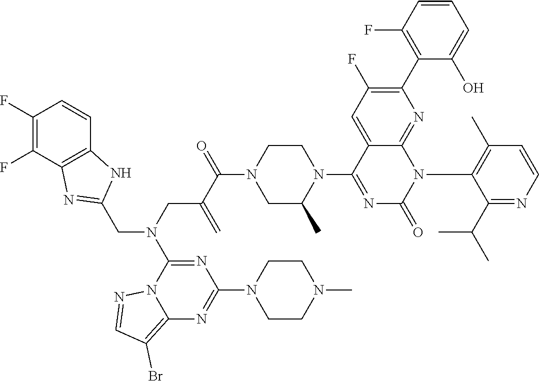 [SMILES: C=C(CN(Cc1nc2c(F)c(F)ccc2[nH]1)c1nc(N2CCN(C)CC2)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-24 | 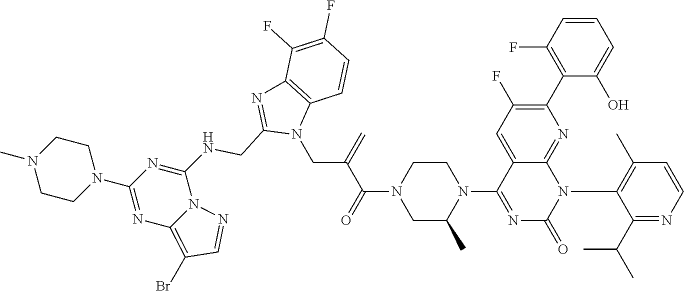 [SMILES: C=C(Cn1c(CNc2nc(N3CCN(C)CC3)nc3c(Br)cnn23)nc2c(F)c(F)ccc21)C(=O)N1CCN(c2nc(=O)n(-c3c(C)ccnc3C(C)C)c3nc(-c4c(O)cccc4F)c(F)cc23)[C@@H](C)C1] |
| II-25 | 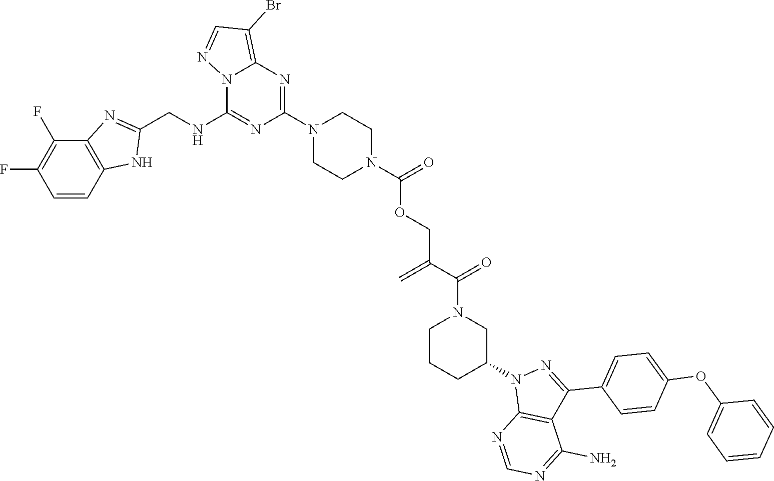 [SMILES: C=C(COC(=O)N1CCN(c2nc(NCc3nc4c(F)c(F)ccc4[nH]3)n3ncc(Br)c3n2)CC1)C(=O)N1CCC[C@@H](n2nc(-c3ccc(Oc4ccccc4)cc3)c3c(N)ncnc32)C1] |
| II-26 | 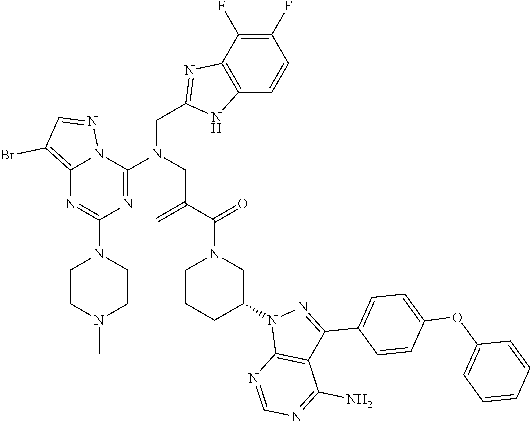 [SMILES: C=C(CN(Cc1nc2c(F)c(F)ccc2[nH]1)c1nc(N2CCN(C)CC2)nc2c(Br)cnn12)C(=O)N1CCC[C@@H](n2nc(-c3ccc(Oc4ccccc4)cc3)c3c(N)ncnc32)C1] |
| II-27 | 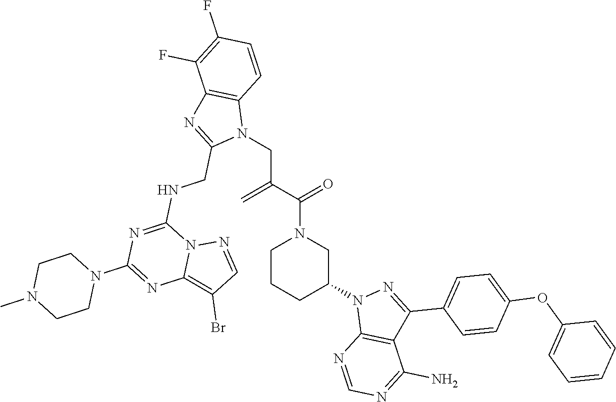 [SMILES: C=C(Cn1c(CNc2nc(N3CCN(C)CC3)nc3c(Br)cnn23)nc2c(F)c(F)ccc21)C(=O)N1CCC[C@@H](n2nc(-c3ccc(Oc4ccccc4)cc3)c3c(N)ncnc32)C1] |
| II-28 | 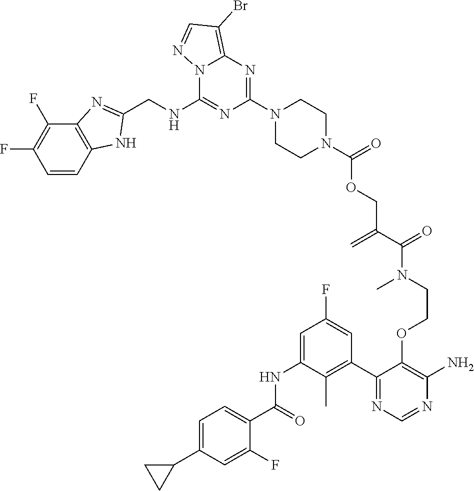 [SMILES: C=C(COC(=O)N1CCN(c2nc(NCc3nc4c(F)c(F)ccc4[nH]3)n3ncc(Br)c3n2)CC1)C(=O)N(C)CCOc1c(N)ncnc1-c1cc(F)cc(NC(=O)c2ccc(C3CC3)cc2F)c1C] |
| II-29 | 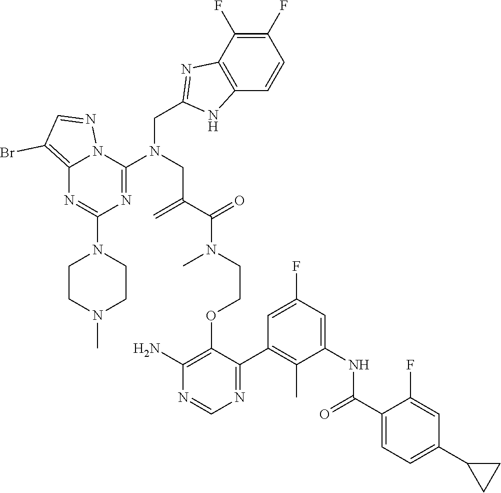 [SMILES: C=C(CN(Cc1nc2c(F)c(F)ccc2[nH]1)c1nc(N2CCN(C)CC2)nc2c(Br)cnn12)C(=O)N(C)CCOc1c(N)ncnc1-c1cc(F)cc(NC(=O)c2ccc(C3CC3)cc2F)c1C] |
| II-30 | 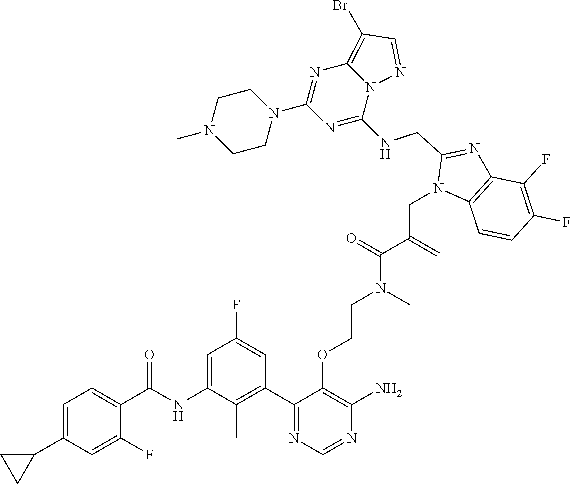 [SMILES: C=C(Cn1c(CNc2nc(N3CCN(C)CC3)nc3c(Br)cnn23)nc2c(F)c(F)ccc21)C(=O)N(C)CCOc1c(N)ncnc1-c1cc(F)cc(NC(=O)c2ccc(C3CC3)cc2F)c1C] |
| II-31 | 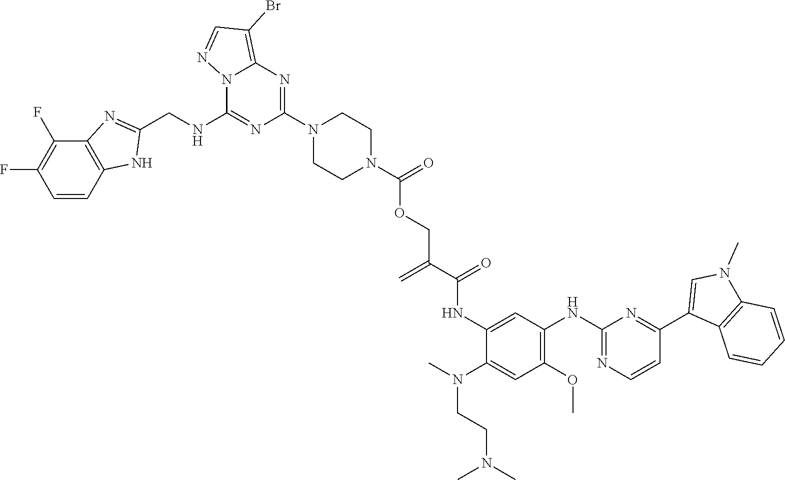 [SMILES: C=C(COC(=O)N1CCN(c2nc(NCc3nc4c(F)c(F)ccc4[nH]3)n3ncc(Br)c3n2)CC1)C(=O)Nc1cc(Nc2nccc(-c3cn(C)c4ccccc34)n2)c(OC)cc1N(C)CCN(C)C] |
| II-32 | 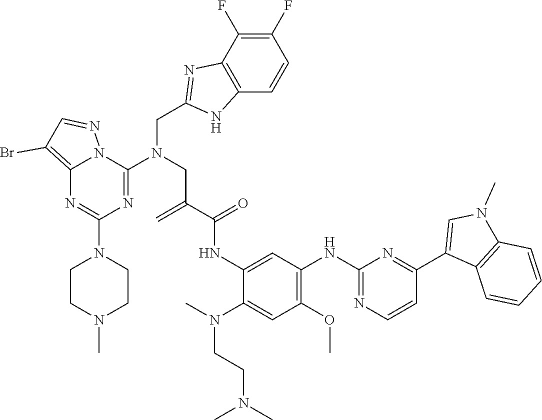 |
| II-33 | 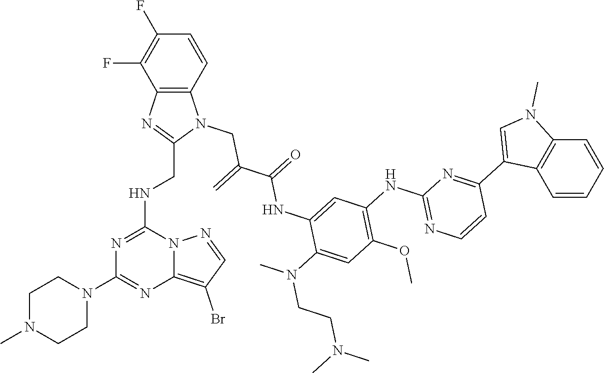 [SMILES: C=C(Cn1c(CNc2nc(N3CCN(C)CC3)nc3c(Br)cnn23)nc2c(F)c(F)ccc21)C(=O)Nc1cc(Nc2nccc(-c3cn(C)c4ccccc34)n2)c(OC)cc1N(C)CCN(C)C] |
| II-34 | 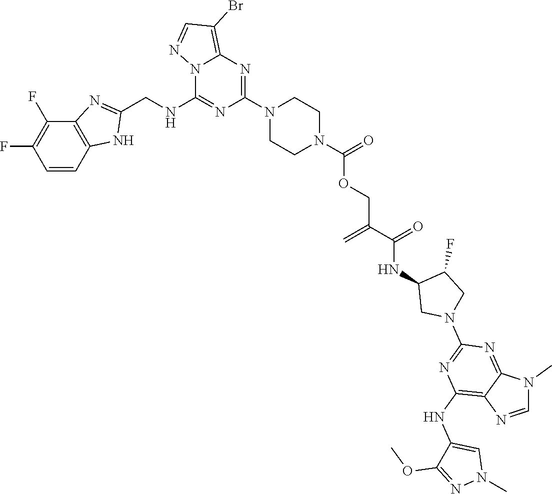 [SMILES: C=C(COC(=O)N1CCN(c2nc(NCc3nc4c(F)c(F)ccc4[nH]3)n3ncc(Br)c3n2)CC1)C(=O)N[C@@H]1CN(c2nc(Nc3cn(C)nc3OC)c3ncn(C)c3n2)C[C@H]1F] |
| II-35 | 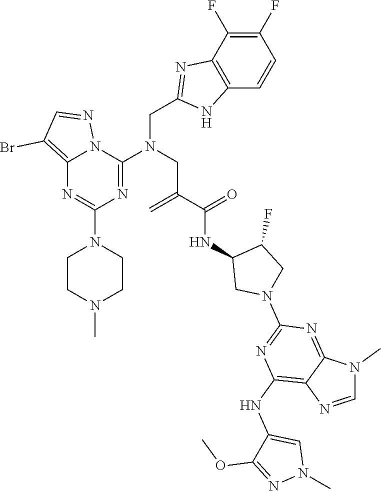 [SMILES: C=C(CN(Cc1nc2c(F)c(F)ccc2[nH]1)c1nc(N2CCN(C)CC2)nc2c(Br)cnn12)C(=O)N[C@@H]1CN(c2nc(Nc3cn(C)nc3OC)c3ncn(C)c3n2)C[C@H]1F] |
| II-36 | 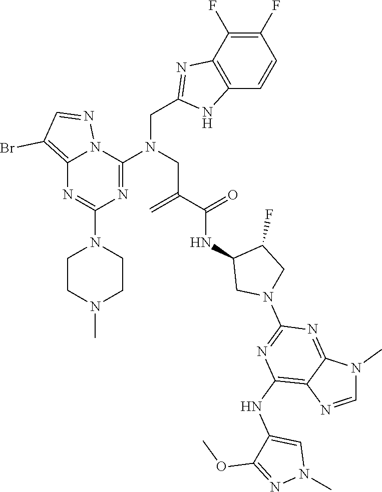 [SMILES: C=C(CN(Cc1nc2c(F)c(F)ccc2[nH]1)c1nc(N2CCN(C)CC2)nc2c(Br)cnn12)C(=O)N[C@@H]1CN(c2nc(Nc3cn(C)nc3OC)c3ncn(C)c3n2)C[C@H]1F] |
| II-37 | 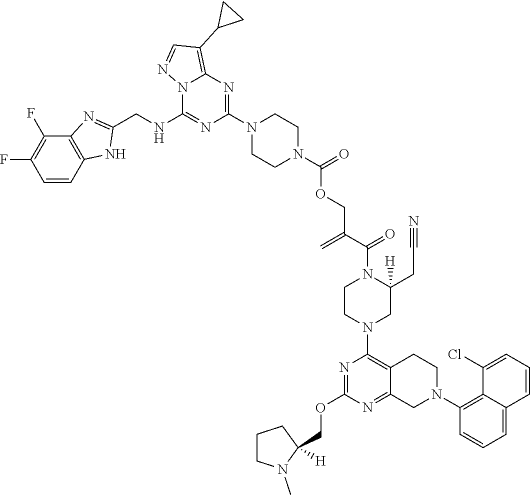 [SMILES: C=C(COC(=O)N1CCN(c2nc(NCc3nc4c(F)c(F)ccc4[nH]3)n3ncc(C4CC4)c3n2)CC1)C(=O)N1CCN(c2nc(OC[C@@H]3CCCN3C)nc3c2CCN(c2cccc4cccc(Cl)c24)C3)C[C@@H]1CC#N] |
| II-38 | 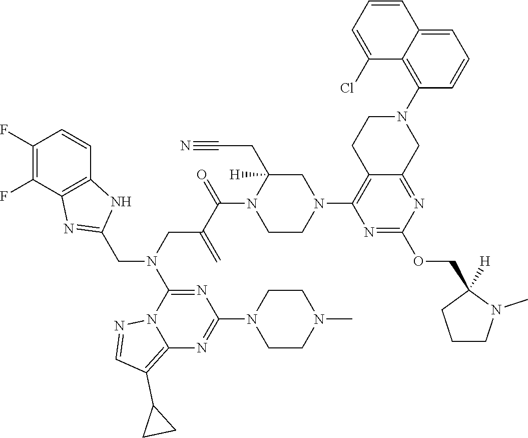 [SMILES: C=C(CN(Cc1nc2c(F)c(F)ccc2[nH]1)c1nc(N2CCN(C)CC2)nc2c(C3CC3)cnn12)C(=O)N1CCN(c2nc(OC[C@@H]3CCCN3C)nc3c2CCN(c2cccc4cccc(Cl)c24)C3)C[C@@H]1CC#N] |
| II-39 | 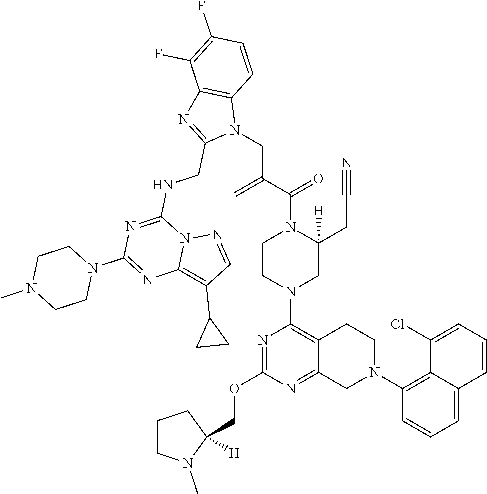 [SMILES: C=C(Cn1c(CNc2nc(N3CCN(C)CC3)nc3c(C4CC4)cnn23)nc2c(F)c(F)ccc21)C(=O)N1CCN(c2nc(OC[C@@H]3CCCN3C)nc3c2CCN(c2cccc4cccc(Cl)c24)C3)C[C@@H]1CC#N] |
| II-40 | 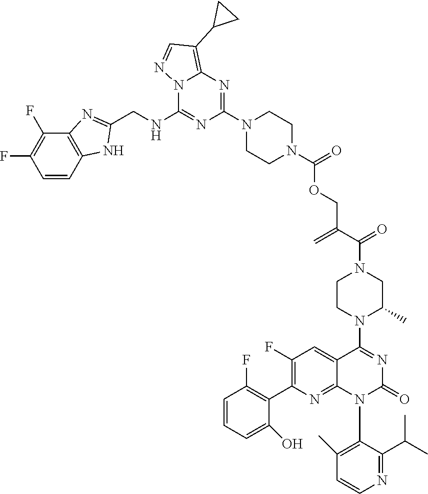 [SMILES: C=C(COC(=O)N1CCN(c2nc(NCc3nc4c(F)c(F)ccc4[nH]3)n3ncc(C4CC4)c3n2)CC1)C(=O)N1CCN(c2nc(=O)n(-c3c(C)ccnc3C(C)C)c3nc(-c4c(O)cccc4F)c(F)cc23)[C@@H](C)C1] |
| II-41 | 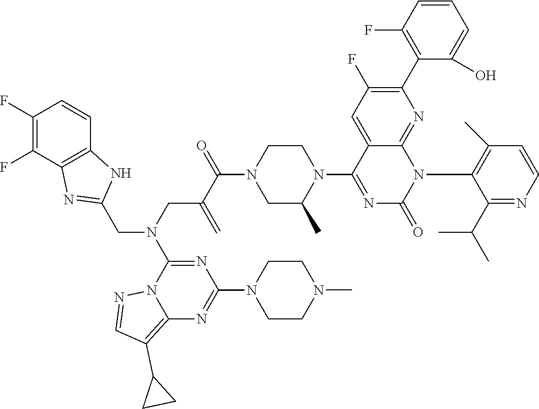 [SMILES: C=C(CN(Cc1nc2c(F)c(F)ccc2[nH]1)c1nc(N2CCN(C)CC2)nc2c(C3CC3)cnn12)C(=O)N1CCN(c2nc(=O)n(-c3c(C)ccnc3C(C)C)c3nc(-c4c(O)cccc4F)c(F)cc23)[C@@H](C)C1] |
| II-42 | 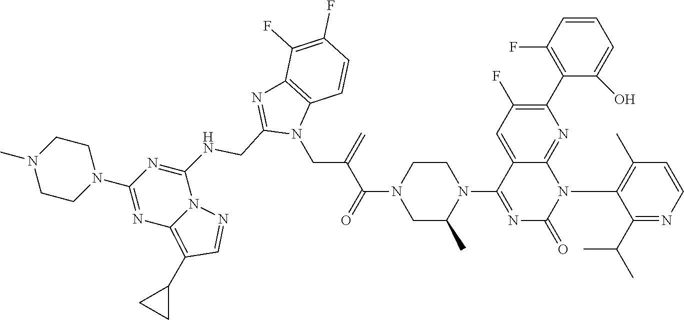 [SMILES: C=C(Cn1c(CNc2nc(N3CCN(C)CC3)nc3c(C4CC4)cnn23)nc2c(F)c(F)ccc21)C(=O)N1CCN(c2nc(=O)n(-c3c(C)ccnc3C(C)C)c3nc(-c4c(O)cccc4F)c(F)cc23)[C@@H](C)C1] |
| II-43 | 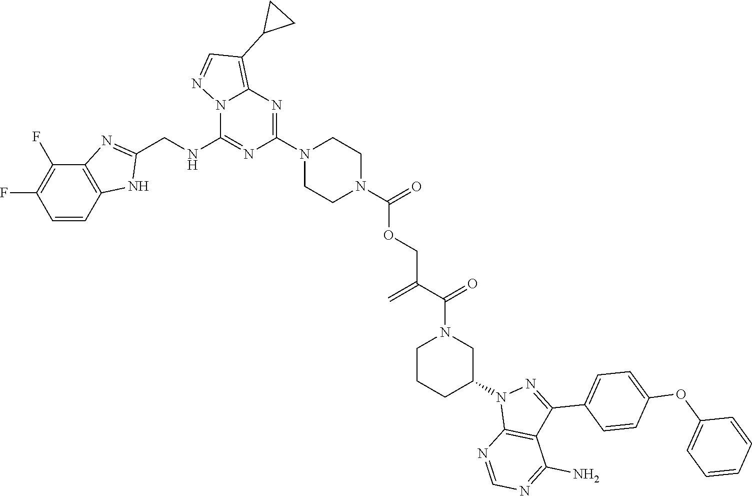 [SMILES: C=C(COC(=O)N1CCN(c2nc(NCc3nc4c(F)c(F)ccc4[nH]3)n3ncc(C4CC4)c3n2)CC1)C(=O)N1CCC[C@@H](n2nc(-c3ccc(Oc4ccccc4)cc3)c3c(N)ncnc32)C1] |
| II-44 | 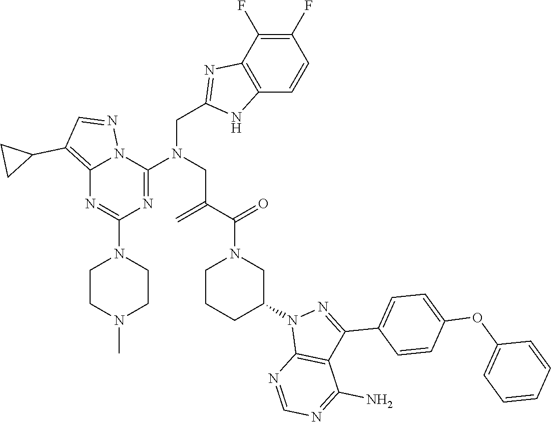 [SMILES: C=C(CN(Cc1nc2c(F)c(F)ccc2[nH]1)c1nc(N2CCN(C)CC2)nc2c(C3CC3)cnn12)C(=O)N1CCC[C@@H](n2nc(-c3ccc(Oc4ccccc4)cc3)c3c(N)ncnc32)C1] |
| II-45 | 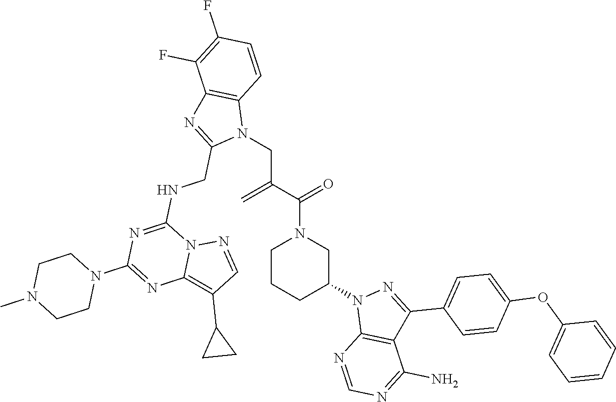 [SMILES: C=C(Cn1c(CNc2nc(N3CCN(C)CC3)nc3c(C4CC4)cnn23)nc2c(F)c(F)ccc21)C(=O)N1CCC[C@@H](n2nc(-c3ccc(Oc4ccccc4)cc3)c3c(N)ncnc32)C1] |
| II-46 | 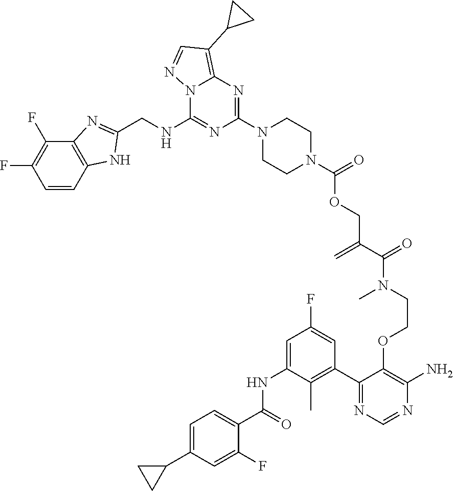 [SMILES: C=C(COC(=O)N1CCN(c2nc(NCc3nc4c(F)c(F)ccc4[nH]3)n3ncc(C4CC4)c3n2)CC1)C(=O)N(C)CCOc1c(N)ncnc1-c1cc(F)cc(NC(=O)c2ccc(C3CC3)cc2F)c1C] |
| II-47 | 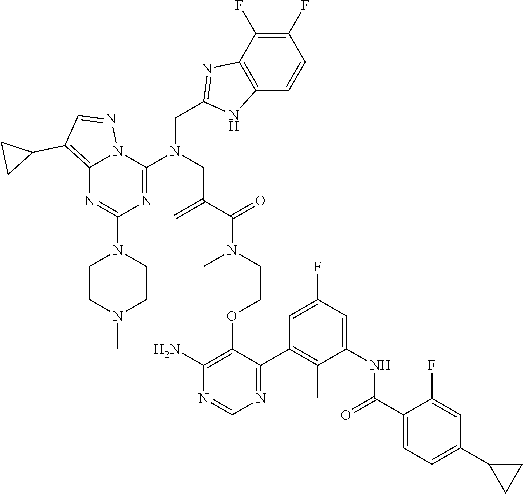 [SMILES: C=C(CN(Cc1nc2c(F)c(F)ccc2[nH]1)c1nc(N2CCN(C)CC2)nc2c(C3CC3)cnn12)C(=O)N(C)CCOc1c(N)ncnc1-c1cc(F)cc(NC(=O)c2ccc(C3CC3)cc2F)c1C] |
| II-48 | 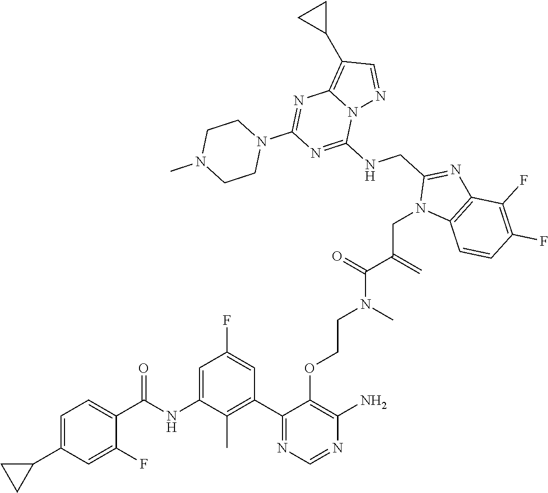 [SMILES: C=C(Cn1c(CNc2nc(N3CCN(C)CC3)nc3c(C4CC4)cnn23)nc2c(F)c(F)ccc21)C(=O)N(C)CCOc1c(N)ncnc1-c1cc(F)cc(NC(=O)c2ccc(C3CC3)cc2F)c1C] |
| II-49 | 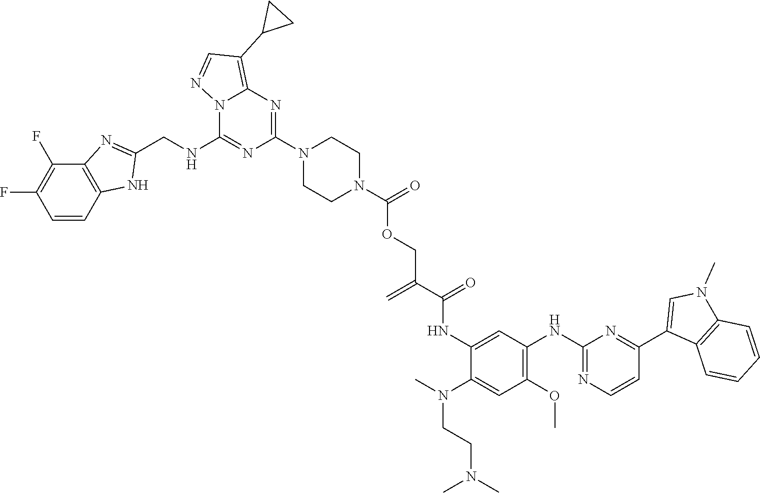 [SMILES: C=C(COC(=O)N1CCN(c2nc(NCc3nc4c(F)c(F)ccc4[nH]3)n3ncc(C4CC4)c3n2)CC1)C(=O)Nc1cc(Nc2nccc(-c3cn(C)c4ccccc34)n2)c(OC)cc1N(C)CCN(C)C] |
| II-50 | 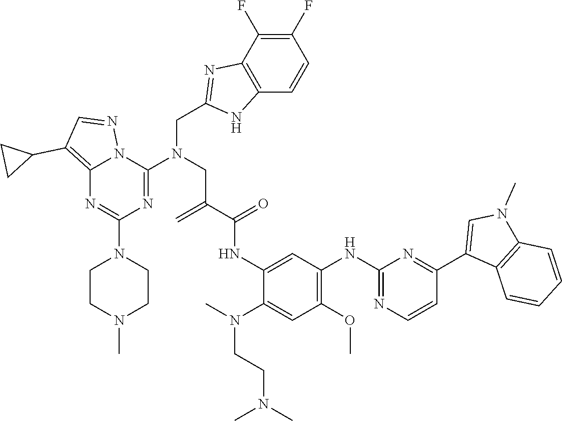 [SMILES: C=C(CN(Cc1nc2c(F)c(F)ccc2[nH]1)c1nc(N2CCN(C)CC2)nc2c(C3CC3)cnn12)C(=O)Nc1cc(Nc2nccc(-c3cn(C)c4ccccc34)n2)c(OC)cc1N(C)CCN(C)C] |
| II-51 | 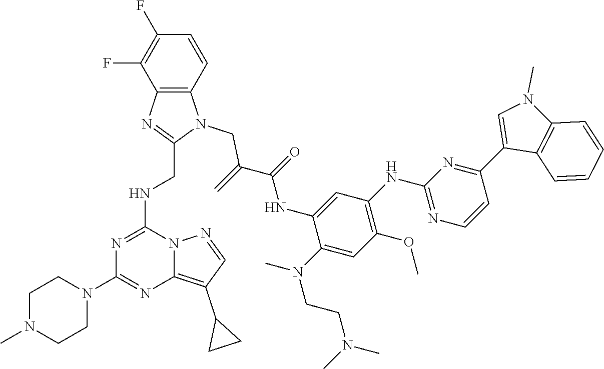 [SMILES: C=C(Cn1c(CNc2nc(N3CCN(C)CC3)nc3c(C4CC4)cnn23)nc2c(F)c(F)ccc21)C(=O)Nc1cc(Nc2nccc(-c3cn(C)c4ccccc34)n2)c(OC)cc1N(C)CCN(C)C] |
| II-52 | 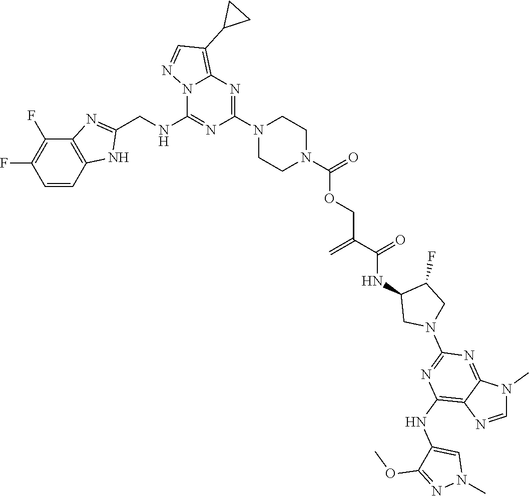 [SMILES: C=C(COC(=O)N1CCN(c2nc(NCc3nc4c(F)c(F)ccc4[nH]3)n3ncc(C4CC4)c3n2)CC1)C(=O)N[C@@H]1CN(c2nc(Nc3cn(C)nc3OC)c3ncn(C)c3n2)C[C@H]1F] |
| II-53 | 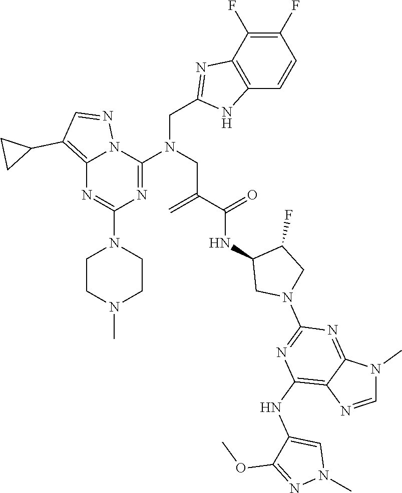 [SMILES: C=C(CN(Cc1nc2c(F)c(F)ccc2[nH]1)c1nc(N2CCN(C)CC2)nc2c(C3CC3)cnn12)C(=O)N[C@@H]1CN(c2nc(Nc3cn(C)nc3OC)c3ncn(C)c3n2)C[C@H]1F] |
| II-54 | 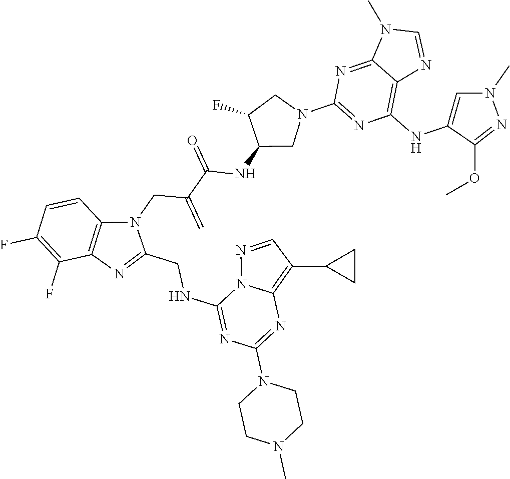 [SMILES: C=C(Cn1c(CNc2nc(N3CCN(C)CC3)nc3c(C4CC4)cnn23)nc2c(F)c(F)ccc21)C(=O)N[C@@H]1CN(c2nc(Nc3cn(C)nc3OC)c3ncn(C)c3n2)C[C@H]1F] |
| II-55 | 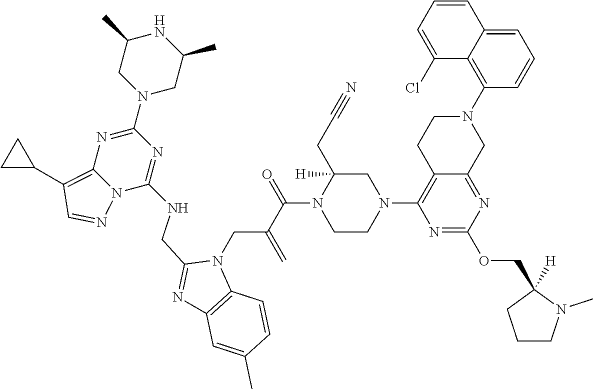 [SMILES: C=C(Cn1c(CNc2nc(N3C[C@@H](C)N[C@@H](C)C3)nc3c(C4CC4)cnn23)nc2cc(C)ccc21)C(=O)N1CCN(c2nc(OC[C@@H]3CCCN3C)nc3c2CCN(c2cccc4cccc(Cl)c24)C3)C[C@@H]1CC#N] |
| II-56 | 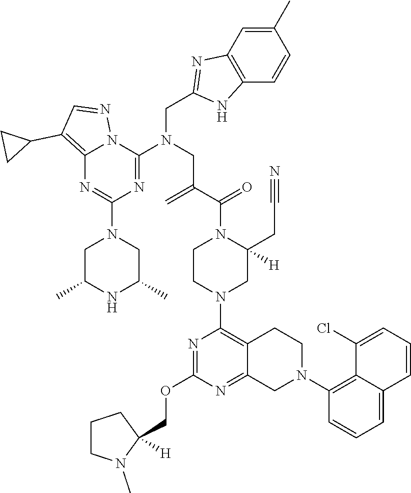 [SMILES: C=C(CN(Cc1nc2cc(C)ccc2[nH]1)c1nc(N2C[C@@H](C)N[C@@H](C)C2)nc2c(C3CC3)cnn12)C(=O)N1CCN(c2nc(OC[C@@H]3CCCN3C)nc3c2CCN(c2cccc4cccc(Cl)c24)C3)C[C@@H]1CC#N] |
| II-57 | 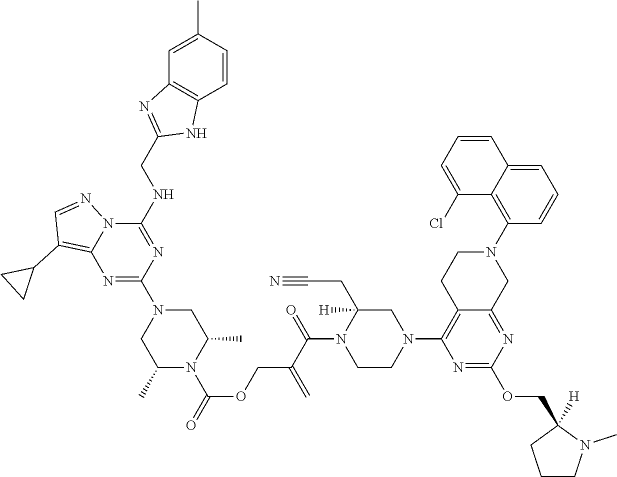 [SMILES: C=C(COC(=O)N1[C@H](C)CN(c2nc(NCc3nc4cc(C)ccc4[nH]3)n3ncc(C4CC4)c3n2)C[C@@H]1C)C(=O)N1CCN(c2nc(OC[C@@H]3CCCN3C)nc3c2CCN(c2cccc4cccc(Cl)c24)C3)C[C@@H]1CC#N] |
| II-58 | 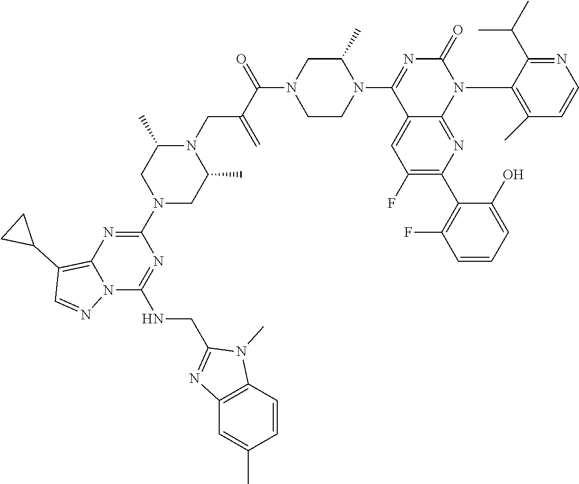 [SMILES: C=C(CN1[C@H](C)CN(c2nc(NCc3nc4cc(C)ccc4n3C)n3ncc(C4CC4)c3n2)C[C@@H]1C)C(=O)N1CCN(c2nc(=O)n(-c3c(C)ccnc3C(C)C)c3nc(-c4c(O)cccc4F)c(F)cc23)[C@@H](C)C1] |
| II-59 | 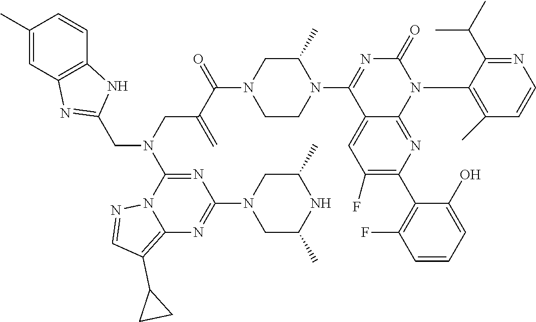 [SMILES: C=C(CN(Cc1nc2cc(C)ccc2[nH]1)c1nc(N2C[C@@H](C)N[C@@H](C)C2)nc2c(C3CC3)cnn12)C(=O)N1CCN(c2nc(=O)n(-c3c(C)ccnc3C(C)C)c3nc(-c4c(O)cccc4F)c(F)cc23)[C@@H](C)C1] |
| II-60 | 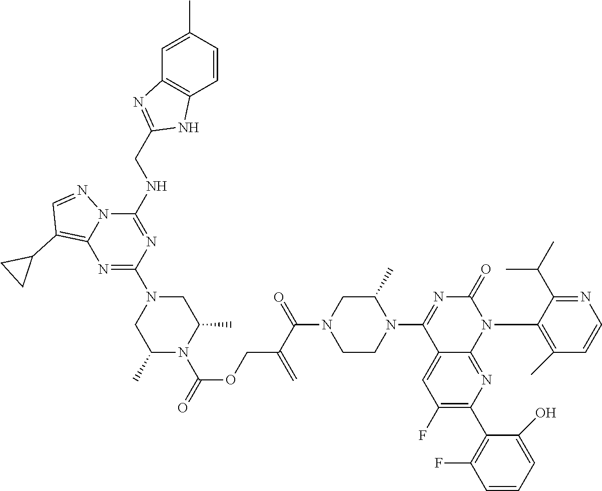 [SMILES: C=C(COC(=O)N1[C@H](C)CN(c2nc(NCc3nc4cc(C)ccc4[nH]3)n3ncc(C4CC4)c3n2)C[C@@H]1C)C(=O)N1CCN(c2nc(=O)n(-c3c(C)ccnc3C(C)C)c3nc(-c4c(O)cccc4F)c(F)cc23)[C@@H](C)C1] |
| II-61 | 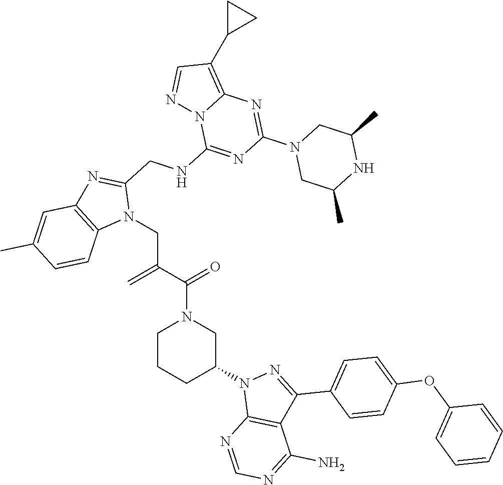 [SMILES: C=C(Cn1c(CNc2nc(N3C[C@@H](C)N[C@@H](C)C3)nc3c(C4CC4)cnn23)nc2cc(C)ccc21)C(=O)N1CCC[C@@H](n2nc(-c3ccc(Oc4ccccc4)cc3)c3c(N)ncnc32)C1] |
| II-62 | 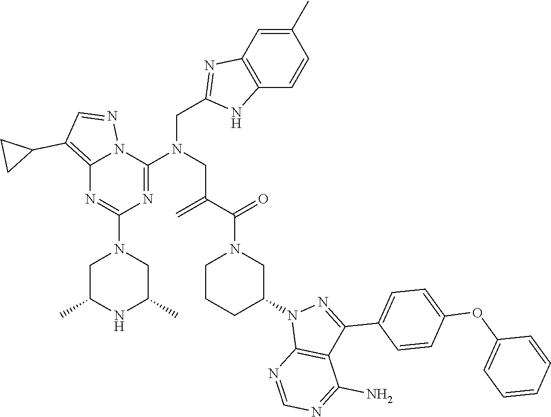 [SMILES: C=C(CN(Cc1nc2cc(C)ccc2[nH]1)c1nc(N2C[C@@H](C)N[C@@H](C)C2)nc2c(C3CC3)cnn12)C(=O)N1CCC[C@@H](n2nc(-c3ccc(Oc4ccccc4)cc3)c3c(N)ncnc32)C1] |
| II-63 | 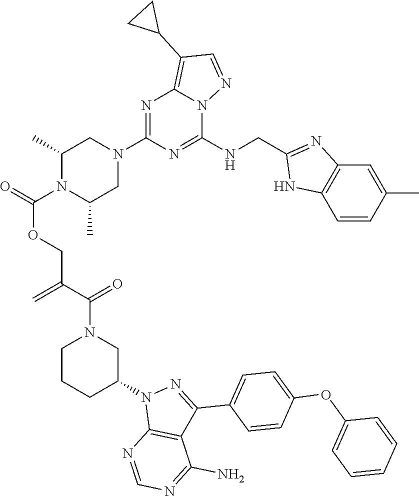 [SMILES: C=C(COC(=O)N1[C@H](C)CN(c2nc(NCc3nc4cc(C)ccc4[nH]3)n3ncc(C4CC4)c3n2)C[C@@H]1C)C(=O)N1CCC[C@@H](n2nc(-c3ccc(Oc4ccccc4)cc3)c3c(N)ncnc32)C1] |
| II-64 | 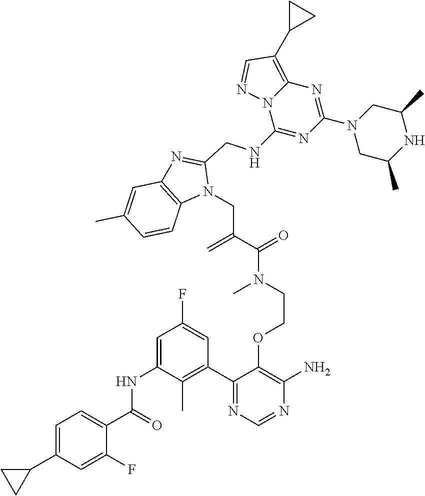 [SMILES: C=C(Cn1c(CNc2nc(N3C[C@@H](C)N[C@@H](C)C3)nc3c(C4CC4)cnn23)nc2cc(C)ccc21)C(=O)N(C)CCOc1c(N)ncnc1-c1cc(F)cc(NC(=O)c2ccc(C3CC3)cc2F)c1C] |
| II-65 | 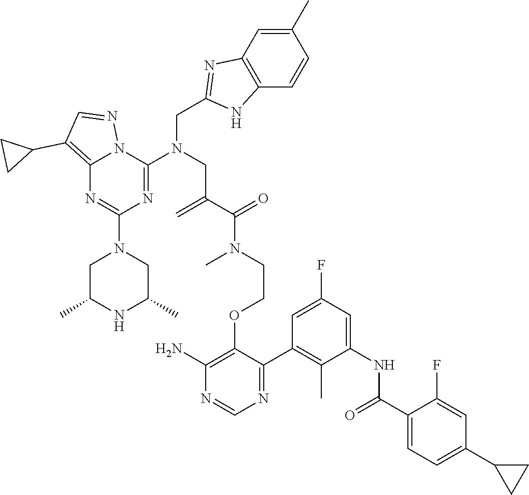 [SMILES: C=C(CN(Cc1nc2cc(C)ccc2[nH]1)c1nc(N2C[C@@H](C)N[C@@H](C)C2)nc2c(C3CC3)cnn12)C(=O)N(C)CCOc1c(N)ncnc1-c1cc(F)cc(NC(=O)c2ccc(C3CC3)cc2F)c1C] |
| II-66 | 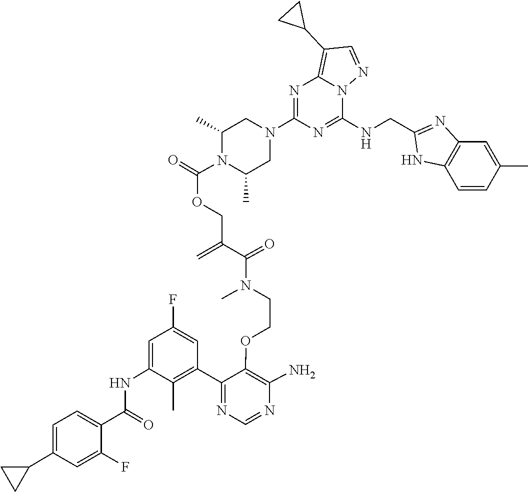 [SMILES: C=C(COC(=O)N1[C@H](C)CN(c2nc(NCc3nc4cc(C)ccc4[nH]3)n3ncc(C4CC4)c3n2)C[C@@H]1C)C(=O)N(C)CCOc1c(N)ncnc1-c1cc(F)cc(NC(=O)c2ccc(C3CC3)cc2F)c1C] |
| II-67 | 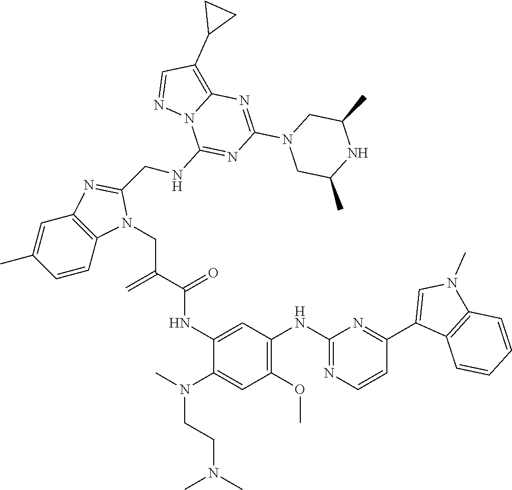 [SMILES: C=C(Cn1c(CNc2nc(N3C[C@@H](C)N[C@@H](C)C3)nc3c(C4CC4)cnn23)nc2cc(C)ccc21)C(=O)Nc1cc(Nc2nccc(-c3cn(C)c4ccccc34)n2)c(OC)cc1N(C)CCN(C)C] |
| II-68 | 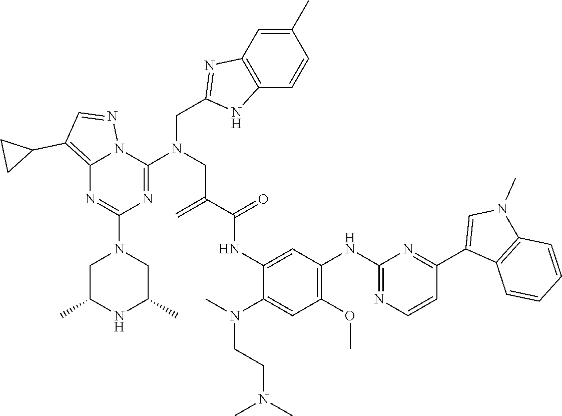 [SMILES: C=C(CN(Cc1nc2cc(C)ccc2[nH]1)c1nc(N2C[C@@H](C)N[C@@H](C)C2)nc2c(C3CC3)cnn12)C(=O)Nc1cc(Nc2nccc(-c3cn(C)c4ccccc34)n2)c(OC)cc1N(C)CCN(C)C] |
| II-69 | 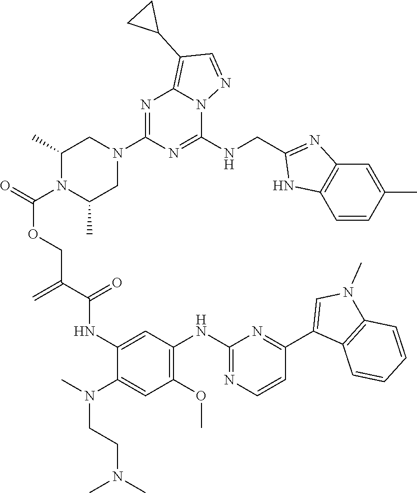 [SMILES: C=C(COC(=O)N1[C@H](C)CN(c2nc(NCc3nc4cc(C)ccc4[nH]3)n3ncc(C4CC4)c3n2)C[C@@H]1C)C(=O)Nc1cc(Nc2nccc(-c3cn(C)c4ccccc34)n2)c(OC)cc1N(C)CCN(C)C] |
| II-70 | 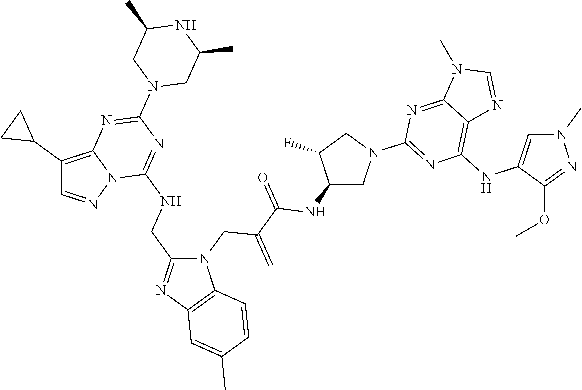 [SMILES: C=C(Cn1c(CNc2nc(N3C[C@@H](C)N[C@@H](C)C3)nc3c(C4CC4)cnn23)nc2cc(C)ccc21)C(=O)N[C@@H]1CN(c2nc(Nc3cn(C)nc3OC)c3ncn(C)c3n2)C[C@H]1F] |
| II-71 | 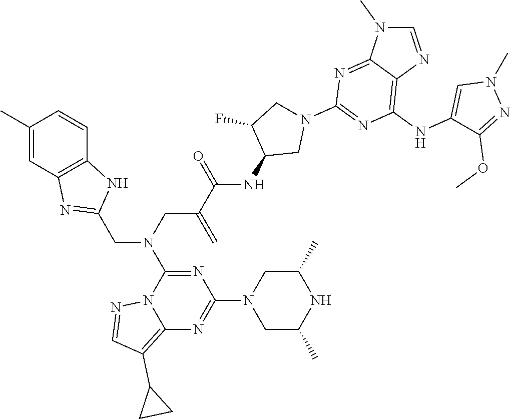 |
| II-72 | 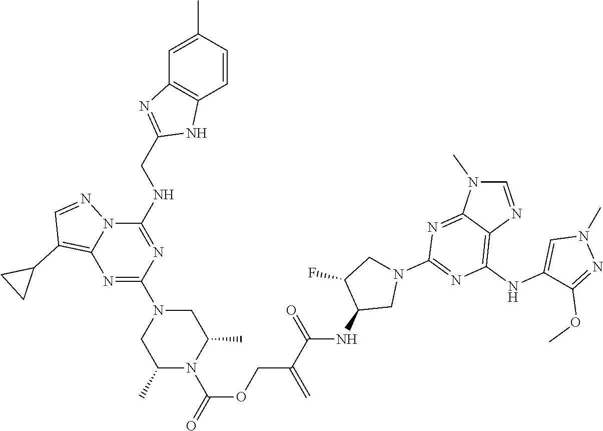 [SMILES: C=C(COC(=O)N1[C@H](C)CN(c2nc(NCc3nc4cc(C)ccc4[nH]3)n3ncc(C4CC4)c3n2)C[C@@H]1C)C(=O)N[C@@H]1CN(c2nc(Nc3cn(C)nc3OC)c3ncn(C)c3n2)C[C@H]1F] |
| II-73 | 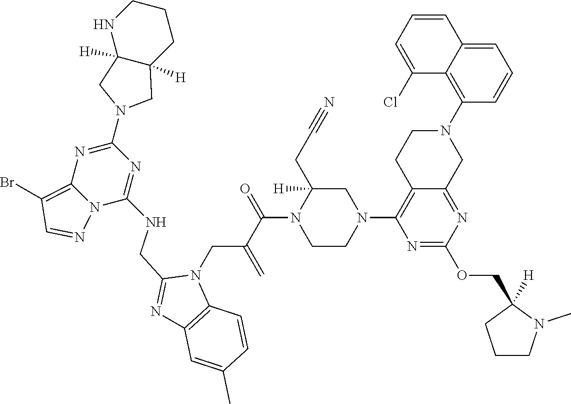 [SMILES: C=C(Cn1c(CNc2nc(N3C[C@@H]4CCCN[C@@H]4C3)nc3c(Br)cnn23)nc2cc(C)ccc21)C(=O)N1CCN(c2nc(OC[C@@H]3CCCN3C)nc3c2CCN(c2cccc4cccc(Cl)c24)C3)C[C@@H]1CC#N] |
| II-74 | 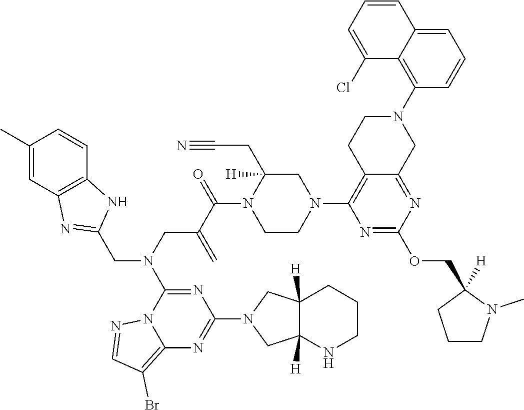 |
| II-75 | 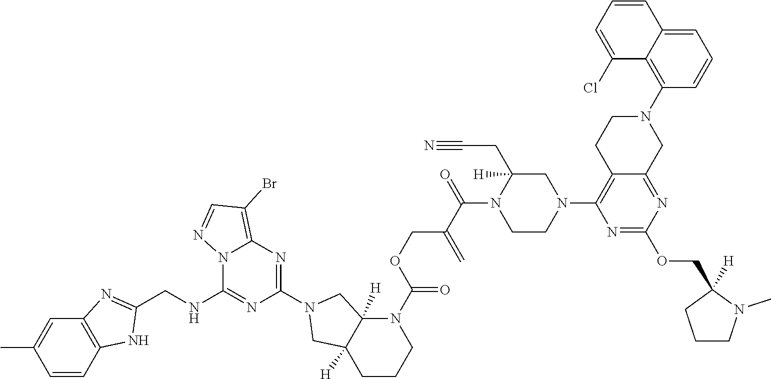 |
| II-76 | 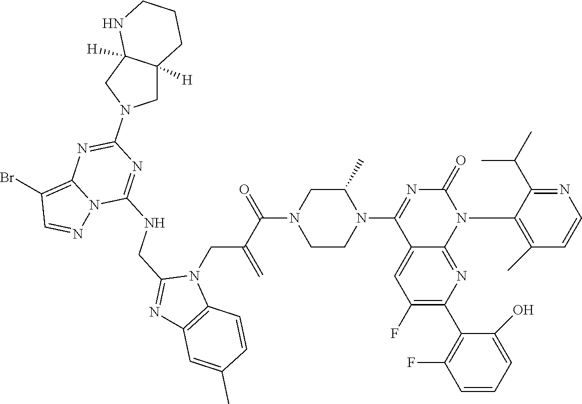 [SMILES: C=C(Cn1c(CNc2nc(N3C[C@@H]4CCCN[C@@H]4C3)nc3c(Br)cnn23)nc2cc(C)ccc21)C(=O)N1CCN(c2nc(=O)n(-c3c(C)ccnc3C(C)C)c3nc(-c4c(O)cccc4F)c(F)cc23)[C@@H](C)C1] |
| II-77 | 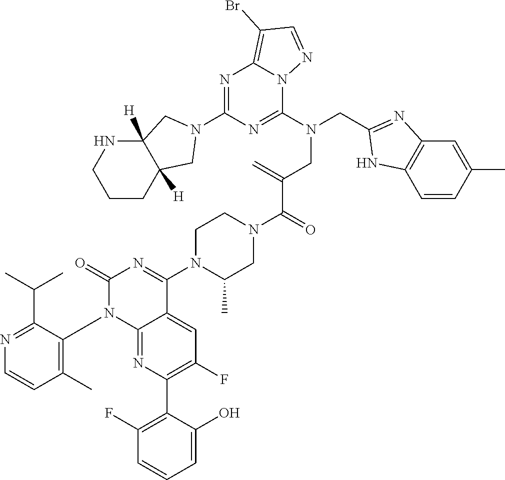 [SMILES: C=C(CN(Cc1nc2cc(C)ccc2[nH]1)c1nc(N2C[C@@H]3CCCN[C@@H]3C2)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-78 | 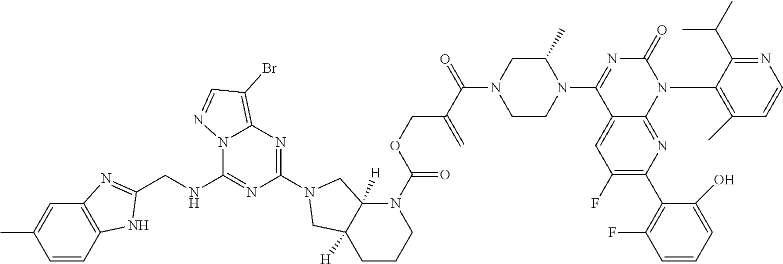 [SMILES: C=C(COC(=O)N1CCC[C@H]2CN(c3nc(NCc4nc5cc(C)ccc5[nH]4)n4ncc(Br)c4n3)C[C@H]21)C(=O)N1CCN(c2nc(=O)n(-c3c(C)ccnc3C(C)C)c3nc(-c4c(O)cccc4F)c(F)cc23)[C@@H](C)C1] |
| II-79 | 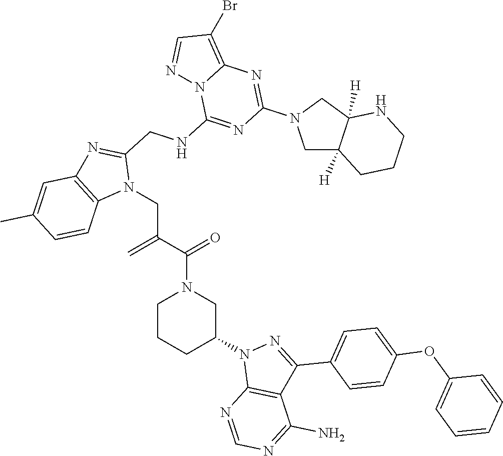 [SMILES: C=C(Cn1c(CNc2nc(N3C[C@@H]4CCCN[C@@H]4C3)nc3c(Br)cnn23)nc2cc(C)ccc21)C(=O)N1CCC[C@@H](n2nc(-c3ccc(Oc4ccccc4)cc3)c3c(N)ncnc32)C1] |
| II-80 | 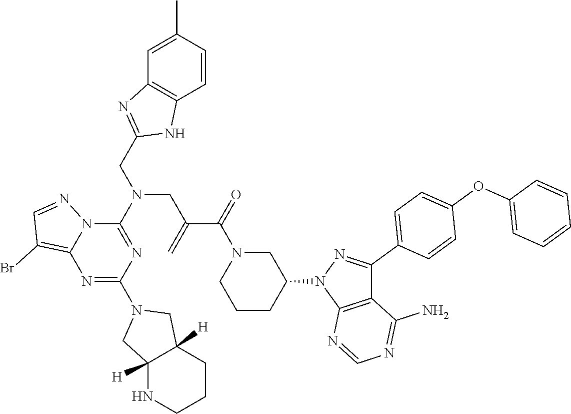 [SMILES: C=C(CN(Cc1nc2cc(C)ccc2[nH]1)c1nc(N2C[C@@H]3CCCN[C@@H]3C2)nc2c(Br)cnn12)C(=O)N1CCC[C@@H](n2nc(-c3ccc(Oc4ccccc4)cc3)c3c(N)ncnc32)C1] |
| II-81 | 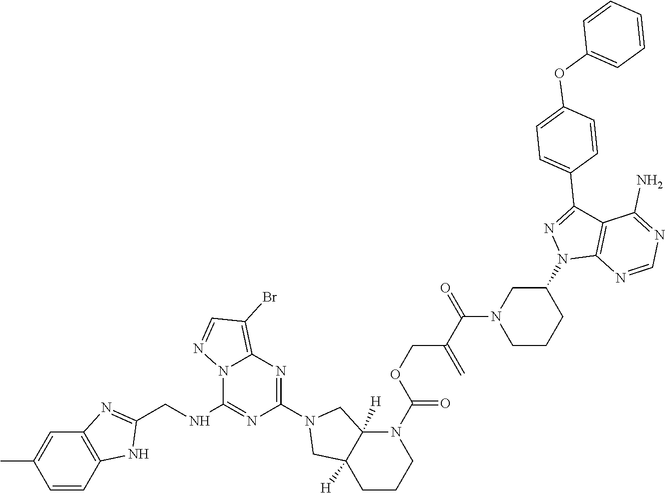 [SMILES: C=C(COC(=O)N1CCC[C@H]2CN(c3nc(NCc4nc5cc(C)ccc5[nH]4)n4ncc(Br)c4n3)C[C@H]21)C(=O)N1CCC[C@@H](n2nc(-c3ccc(Oc4ccccc4)cc3)c3c(N)ncnc32)C1] |
| II-82 | 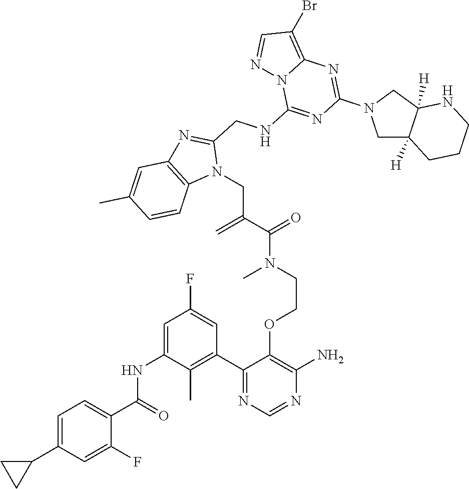 [SMILES: C=C(Cn1c(CNc2nc(N3C[C@@H]4CCCN[C@@H]4C3)nc3c(Br)cnn23)nc2cc(C)ccc21)C(=O)N(C)CCOc1c(N)ncnc1-c1cc(F)cc(NC(=O)c2ccc(C3CC3)cc2F)c1C] |
| II-83 | 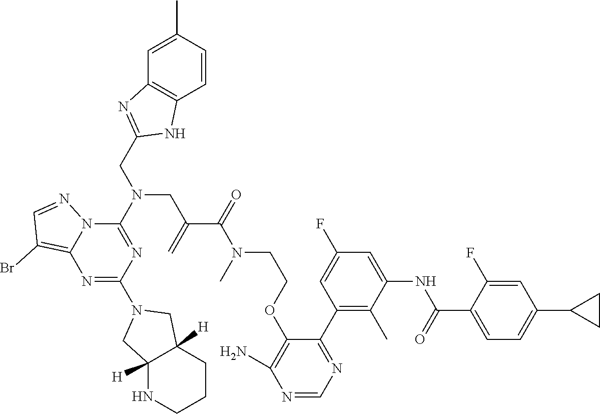 [SMILES: C=C(CN(Cc1nc2cc(C)ccc2[nH]1)c1nc(N2C[C@@H]3CCCN[C@@H]3C2)nc2c(Br)cnn12)C(=O)N(C)CCOc1c(N)ncnc1-c1cc(F)cc(NC(=O)c2ccc(C3CC3)cc2F)c1C] |
| II-84 | 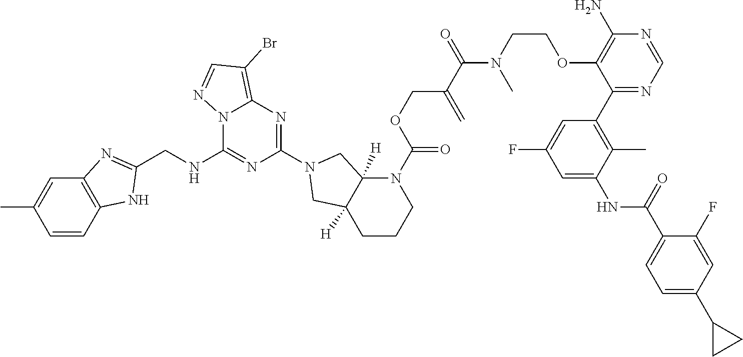 [SMILES: C=C(COC(=O)N1CCC[C@H]2CN(c3nc(NCc4nc5cc(C)ccc5[nH]4)n4ncc(Br)c4n3)C[C@H]21)C(=O)N(C)CCOc1c(N)ncnc1-c1cc(F)cc(NC(=O)c2ccc(C3CC3)cc2F)c1C] |
| II-85 | 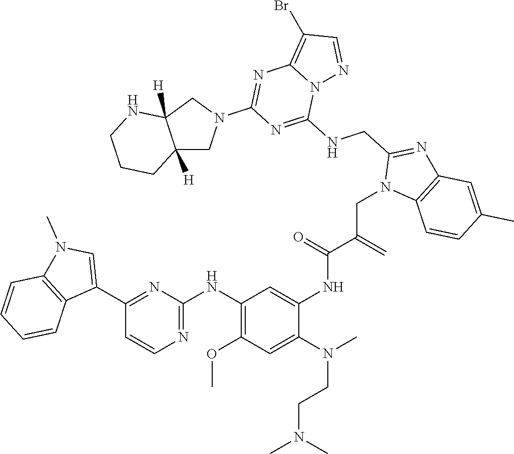 [SMILES: C=C(Cn1c(CNc2nc(N3C[C@@H]4CCCN[C@@H]4C3)nc3c(Br)cnn23)nc2cc(C)ccc21)C(=O)Nc1cc(Nc2nccc(-c3cn(C)c4ccccc34)n2)c(OC)cc1N(C)CCN(C)C] |
| II-86 | 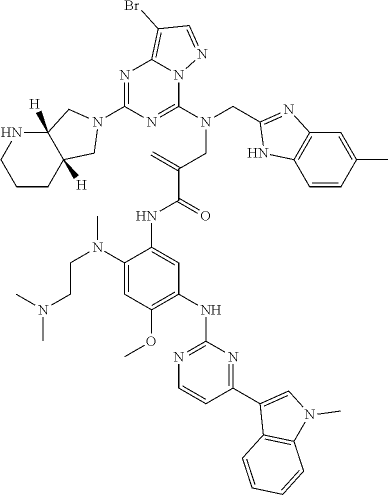 [SMILES: C=C(CN(Cc1nc2cc(C)ccc2[nH]1)c1nc(N2C[C@@H]3CCCN[C@@H]3C2)nc2c(Br)cnn12)C(=O)Nc1cc(Nc2nccc(-c3cn(C)c4ccccc34)n2)c(OC)cc1N(C)CCN(C)C] |
| II-87 | 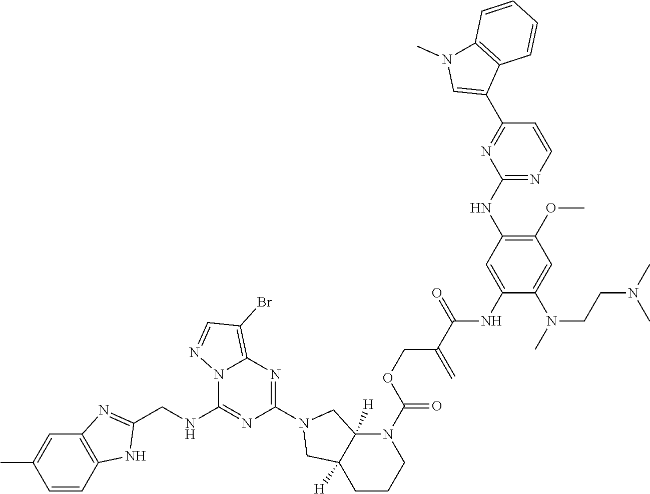 |
| II-88 | 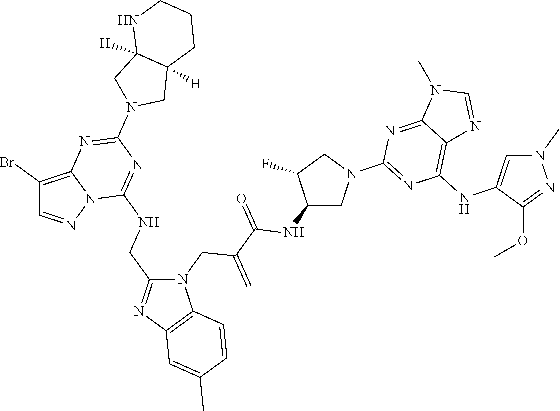 |
| II-89 | 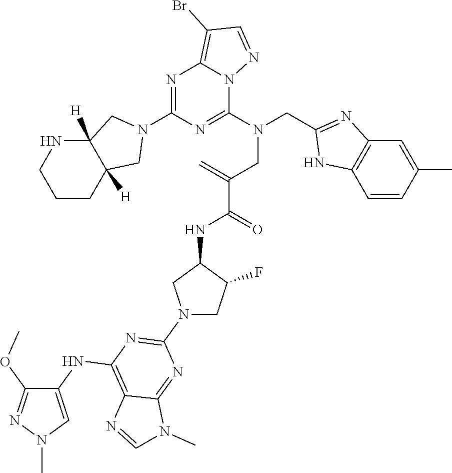 [SMILES: C=C(CN(Cc1nc2cc(C)ccc2[nH]1)c1nc(N2C[C@@H]3CCCN[C@@H]3C2)nc2c(Br)cnn12)C(=O)N[C@@H]1CN(c2nc(Nc3cn(C)nc3OC)c3ncn(C)c3n2)C[C@H]1F] |
| II-90 | 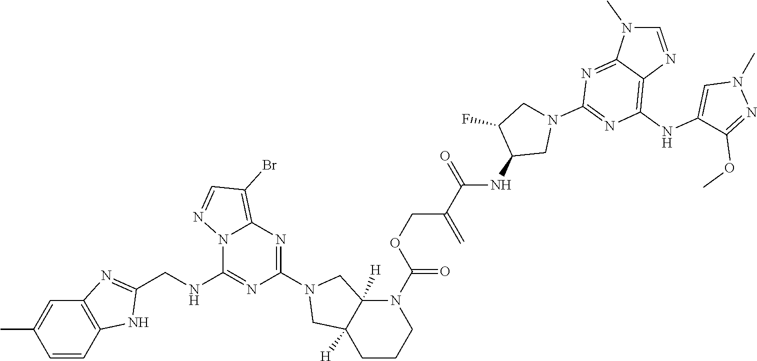 |
| II-91 | 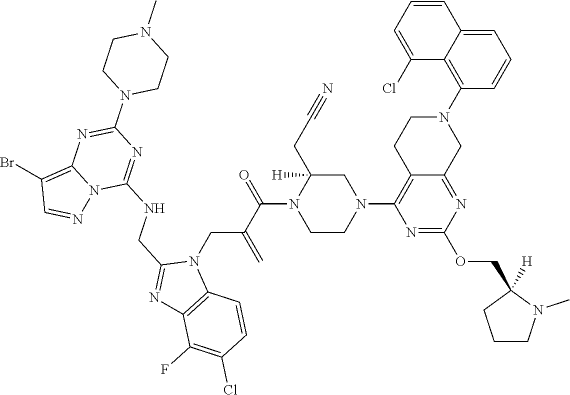 [SMILES: C=C(Cn1c(CNc2nc(N3CCN(C)CC3)nc3c(Br)cnn23)nc2c(F)c(Cl)ccc21)C(=O)N1CCN(c2nc(OC[C@@H]3CCCN3C)nc3c2CCN(c2cccc4cccc(Cl)c24)C3)C[C@@H]1CC#N] |
| II-92 | 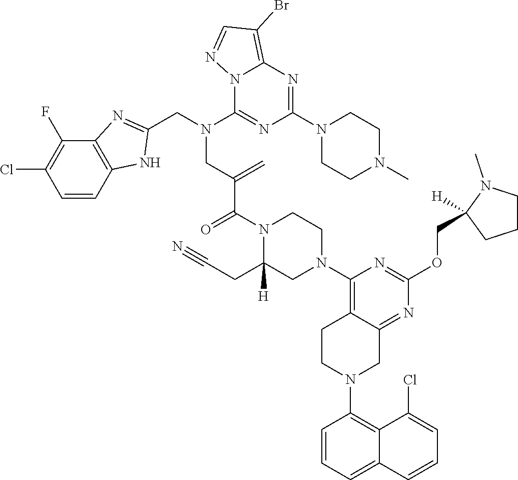 [SMILES: C=C(CN(Cc1nc2c(F)c(Cl)ccc2[nH]1)c1nc(N2CCN(C)CC2)nc2c(Br)cnn12)C(=O)N1CCN(c2nc(OC[C@@H]3CCCN3C)nc3c2CCN(c2cccc4cccc(Cl)c24)C3)C[C@@H]1CC#N] |
| II-93 | 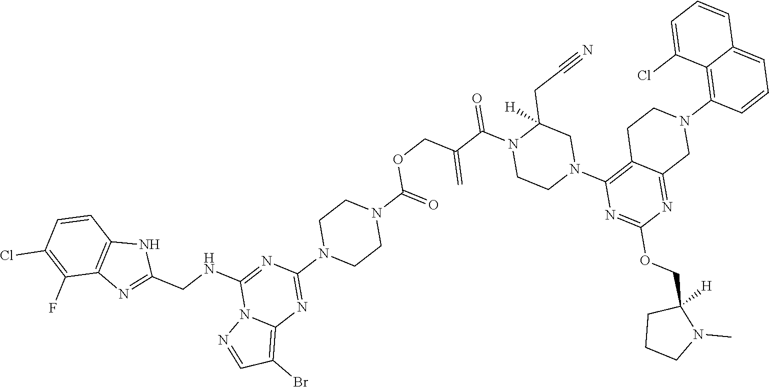 [SMILES: C=C(COC(=O)N1CCN(c2nc(NCc3nc4c(F)c(Cl)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| II-94 | 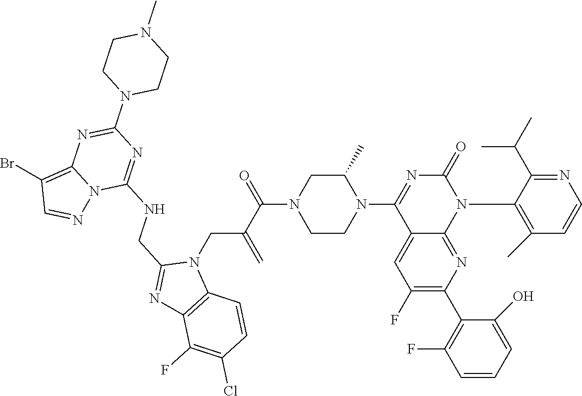 [SMILES: C=C(Cn1c(CNc2nc(N3CCN(C)CC3)nc3c(Br)cnn23)nc2c(F)c(Cl)ccc21)C(=O)N1CCN(c2nc(=O)n(-c3c(C)ccnc3C(C)C)c3nc(-c4c(O)cccc4F)c(F)cc23)[C@@H](C)C1] |
| II-95 | 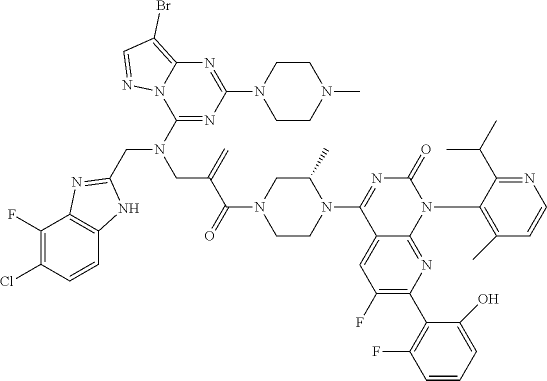 [SMILES: C=C(CN(Cc1nc2c(F)c(Cl)ccc2[nH]1)c1nc(N2CCN(C)CC2)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-96 | 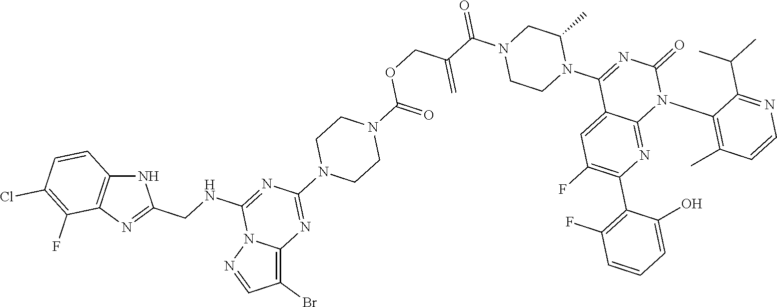 [SMILES: C=C(COC(=O)N1CCN(c2nc(NCc3nc4c(F)c(Cl)ccc4[nH]3)n3ncc(Br)c3n2)CC1)C(=O)N1CCN(c2nc(=O)n(-c3c(C)ccnc3C(C)C)c3nc(-c4c(O)cccc4F)c(F)cc23)[C@@H](C)C1] |
| II-97 | 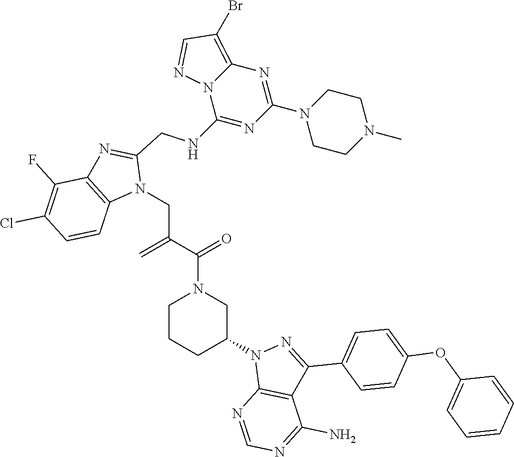 [SMILES: C=C(Cn1c(CNc2nc(N3CCN(C)CC3)nc3c(Br)cnn23)nc2c(F)c(Cl)ccc21)C(=O)N1CCC[C@@H](n2nc(-c3ccc(Oc4ccccc4)cc3)c3c(N)ncnc32)C1] |
| II-98 | 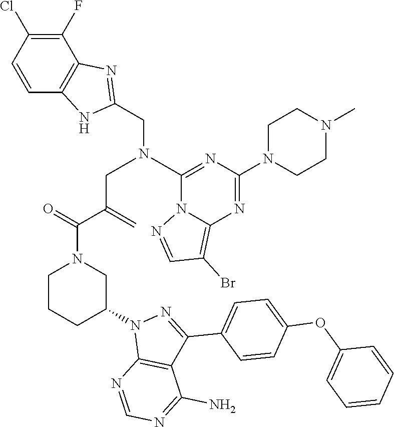 [SMILES: C=C(CN(Cc1nc2c(F)c(Cl)ccc2[nH]1)c1nc(N2CCN(C)CC2)nc2c(Br)cnn12)C(=O)N1CCC[C@@H](n2nc(-c3ccc(Oc4ccccc4)cc3)c3c(N)ncnc32)C1] |
| II-99 | 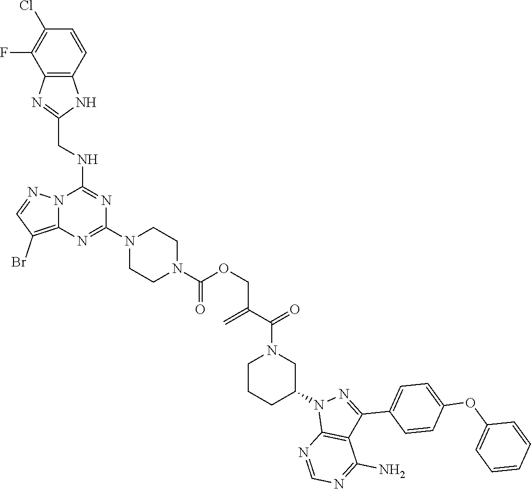 [SMILES: C=C(COC(=O)N1CCN(c2nc(NCc3nc4c(F)c(Cl)ccc4[nH]3)n3ncc(Br)c3n2)CC1)C(=O)N1CCC[C@@H](n2nc(-c3ccc(Oc4ccccc4)cc3)c3c(N)ncnc32)C1] |
| II-100 | 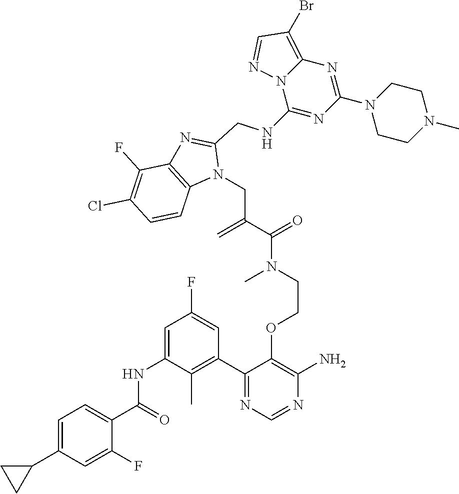 [SMILES: C=C(Cn1c(CNc2nc(N3CCN(C)CC3)nc3c(Br)cnn23)nc2c(F)c(Cl)ccc21)C(=O)N(C)CCOc1c(N)ncnc1-c1cc(F)cc(NC(=O)c2ccc(C3CC3)cc2F)c1C] |
| II-101 | 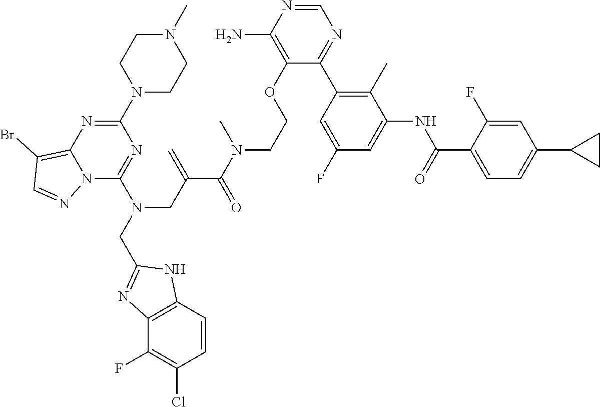 [SMILES: C=C(CN(Cc1nc2c(F)c(Cl)ccc2[nH]1)c1nc(N2CCN(C)CC2)nc2c(Br)cnn12)C(=O)N(C)CCOc1c(N)ncnc1-c1cc(F)cc(NC(=O)c2ccc(C3CC3)cc2F)c1C] |
| II-102 | 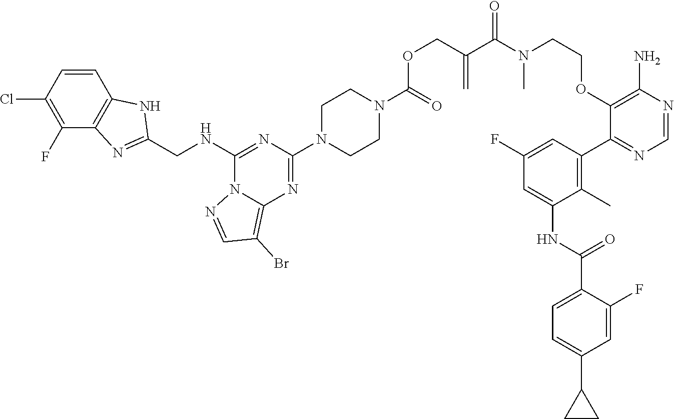 [SMILES: C=C(COC(=O)N1CCN(c2nc(NCc3nc4c(F)c(Cl)ccc4[nH]3)n3ncc(Br)c3n2)CC1)C(=O)N(C)CCOc1c(N)ncnc1-c1cc(F)cc(NC(=O)c2ccc(C3CC3)cc2F)c1C] |
| II-103 | 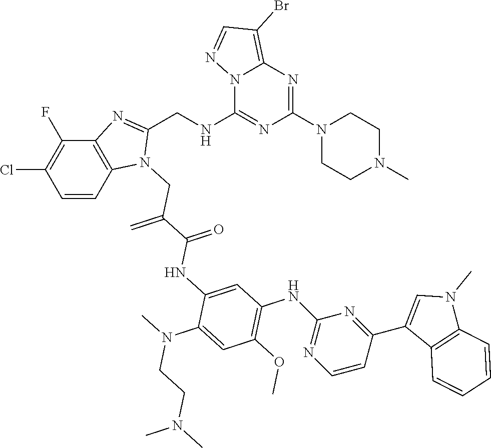 [SMILES: C=C(Cn1c(CNc2nc(N3CCN(C)CC3)nc3c(Br)cnn23)nc2c(F)c(Cl)ccc21)C(=O)Nc1cc(Nc2nccc(-c3cn(C)c4ccccc34)n2)c(OC)cc1N(C)CCN(C)C] |
| II-104 | 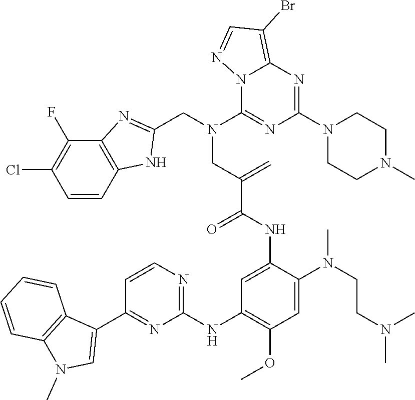 [SMILES: C=C(CN(Cc1nc2c(F)c(Cl)ccc2[nH]1)c1nc(N2CCN(C)CC2)nc2c(Br)cnn12)C(=O)Nc1cc(Nc2nccc(-c3cn(C)c4ccccc34)n2)c(OC)cc1N(C)CCN(C)C] |
| II-105 | 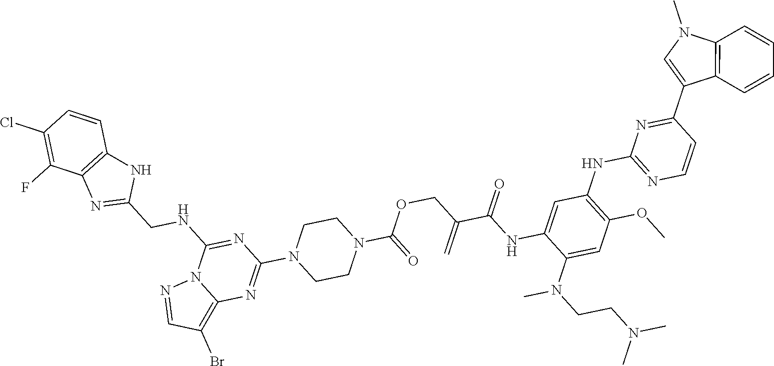 [SMILES: C=C(COC(=O)N1CCN(c2nc(NCc3nc4c(F)c(Cl)ccc4[nH]3)n3ncc(Br)c3n2)CC1)C(=O)Nc1cc(Nc2nccc(-c3cn(C)c4ccccc34)n2)c(OC)cc1N(C)CCN(C)C] |
| II-106 | 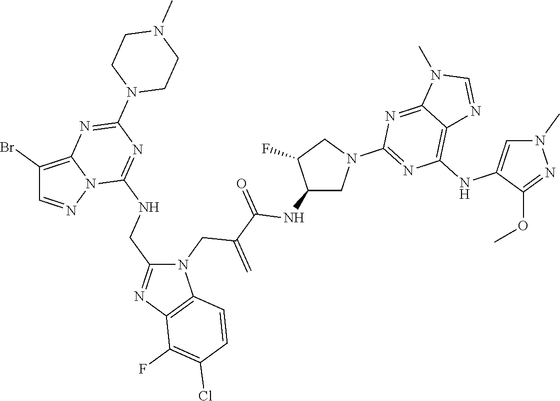 [SMILES: C=C(Cn1c(CNc2nc(N3CCN(C)CC3)nc3c(Br)cnn23)nc2c(F)c(Cl)ccc21)C(=O)N[C@@H]1CN(c2nc(Nc3cn(C)nc3OC)c3ncn(C)c3n2)C[C@H]1F] |
| II-107 | 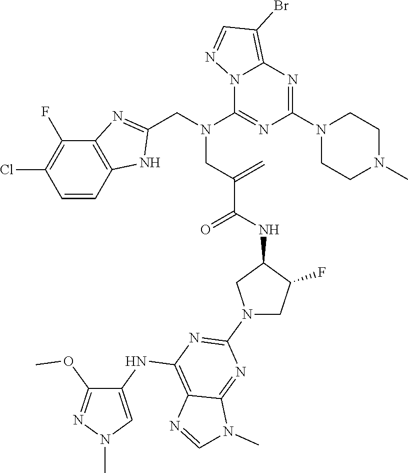 [SMILES: C=C(CN(Cc1nc2c(F)c(Cl)ccc2[nH]1)c1nc(N2CCN(C)CC2)nc2c(Br)cnn12)C(=O)N[C@@H]1CN(c2nc(Nc3cn(C)nc3OC)c3ncn(C)c3n2)C[C@H]1F] |
| II-108 | 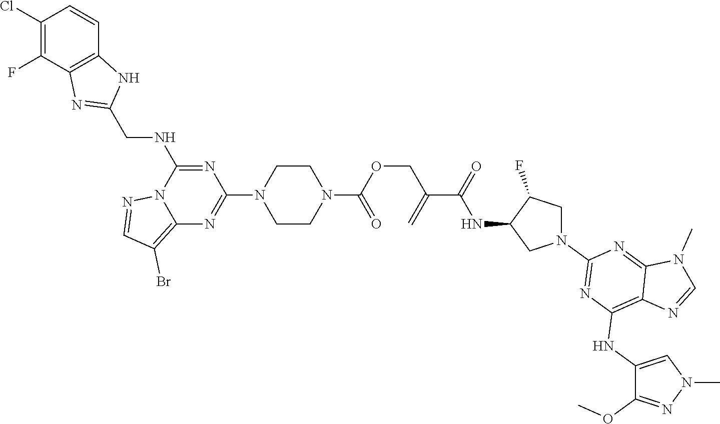 [SMILES: C=C(COC(=O)N1CCN(c2nc(NCc3nc4c(F)c(Cl)ccc4[nH]3)n3ncc(Br)c3n2)CC1)C(=O)N[C@@H]1CN(c2nc(Nc3cn(C)nc3OC)c3ncn(C)c3n2)C[C@H]1F] |
| II-109 | 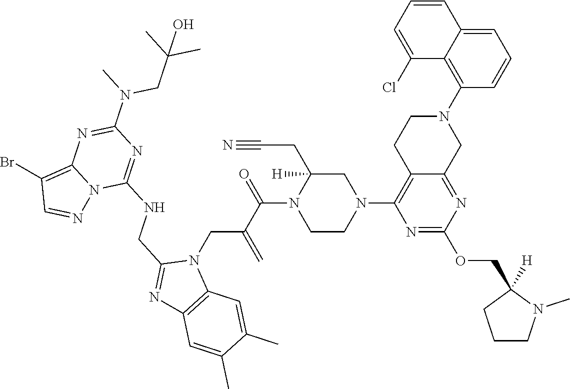 [SMILES: C=C(Cn1c(CNc2nc(N(C)CC(C)(C)O)nc3c(Br)cnn23)nc2cc(C)c(C)cc21)C(=O)N1CCN(c2nc(OC[C@@H]3CCCN3C)nc3c2CCN(c2cccc4cccc(Cl)c24)C3)C[C@@H]1CC#N] |
| II-110 | 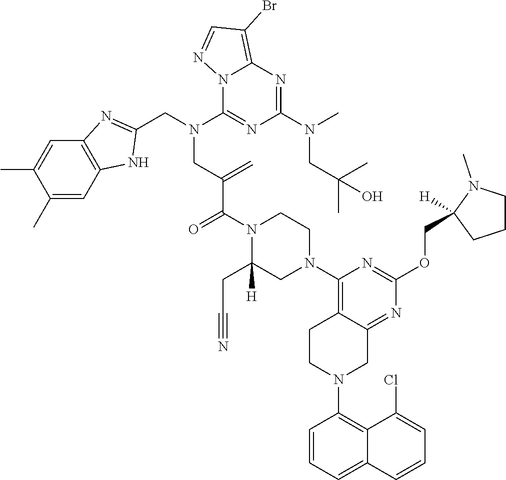 [SMILES: C=C(CN(Cc1nc2cc(C)c(C)cc2[nH]1)c1nc(N(C)CC(C)(C)O)nc2c(Br)cnn12)C(=O)N1CCN(c2nc(OC[C@@H]3CCCN3C)nc3c2CCN(c2cccc4cccc(Cl)c24)C3)C[C@@H]1CC#N] |
| II-111 | 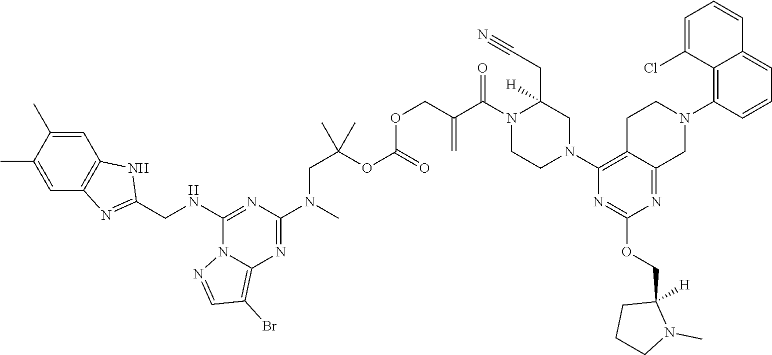 [SMILES: C=C(COC(=O)OC(C)(C)CN(C)c1nc(NCc2nc3cc(C)c(C)cc3[nH]2)n2ncc(Br)c2n1)C(=O)N1CCN(c2nc(OC[C@@H]3CCCN3C)nc3c2CCN(c2cccc4cccc(Cl)c24)C3)C[C@@H]1CC#N] |
| II-112 | 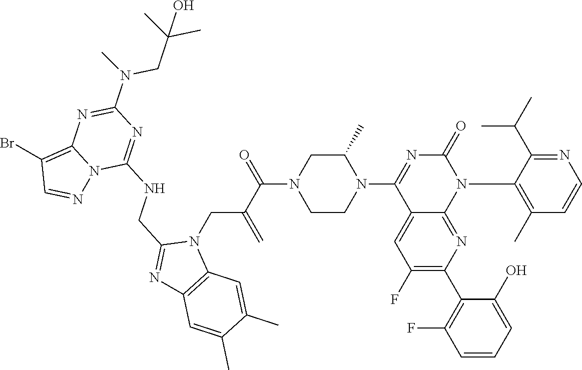 [SMILES: C=C(Cn1c(CNc2nc(N(C)CC(C)(C)O)nc3c(Br)cnn23)nc2cc(C)c(C)cc21)C(=O)N1CCN(c2nc(=O)n(-c3c(C)ccnc3C(C)C)c3nc(-c4c(O)cccc4F)c(F)cc23)[C@@H](C)C1] |
| II-113 | 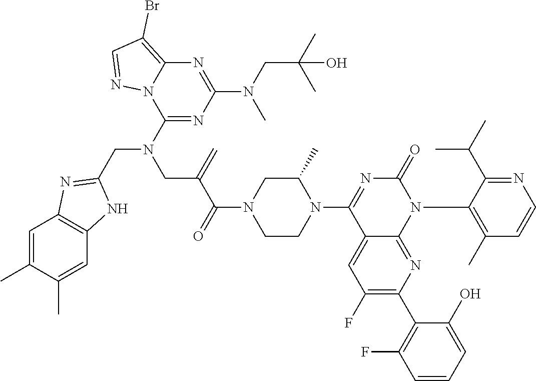 [SMILES: C=C(CN(Cc1nc2cc(C)c(C)cc2[nH]1)c1nc(N(C)CC(C)(C)O)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-114 | 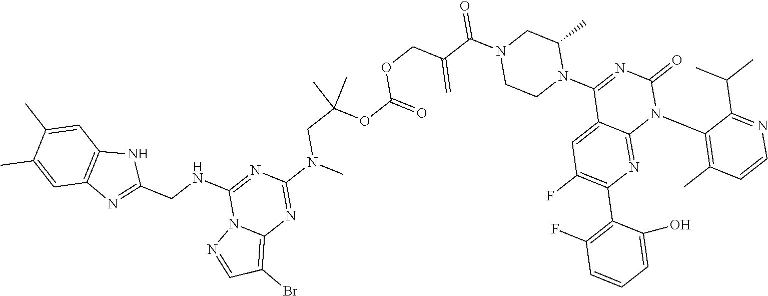 [SMILES: C=C(COC(=O)OC(C)(C)CN(C)c1nc(NCc2nc3cc(C)c(C)cc3[nH]2)n2ncc(Br)c2n1)C(=O)N1CCN(c2nc(=O)n(-c3c(C)ccnc3C(C)C)c3nc(-c4c(O)cccc4F)c(F)cc23)[C@@H](C)C1] |
| II-115 | 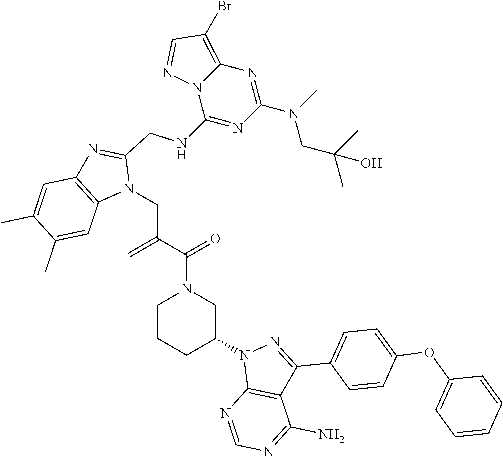 [SMILES: C=C(Cn1c(CNc2nc(N(C)CC(C)(C)O)nc3c(Br)cnn23)nc2cc(C)c(C)cc21)C(=O)N1CCC[C@@H](n2nc(-c3ccc(Oc4ccccc4)cc3)c3c(N)ncnc32)C1] |
| II-116 | 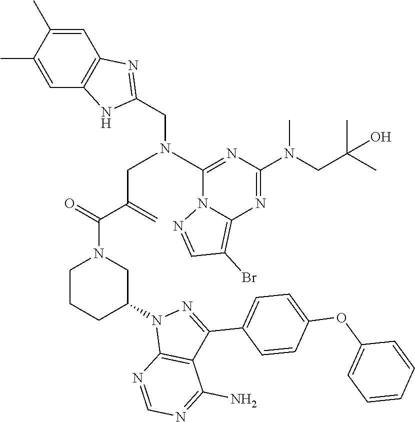 [SMILES: C=C(CN(Cc1nc2cc(C)c(C)cc2[nH]1)c1nc(N(C)CC(C)(C)O)nc2c(Br)cnn12)C(=O)N1CCC[C@@H](n2nc(-c3ccc(Oc4ccccc4)cc3)c3c(N)ncnc32)C1] |
| II-117 | 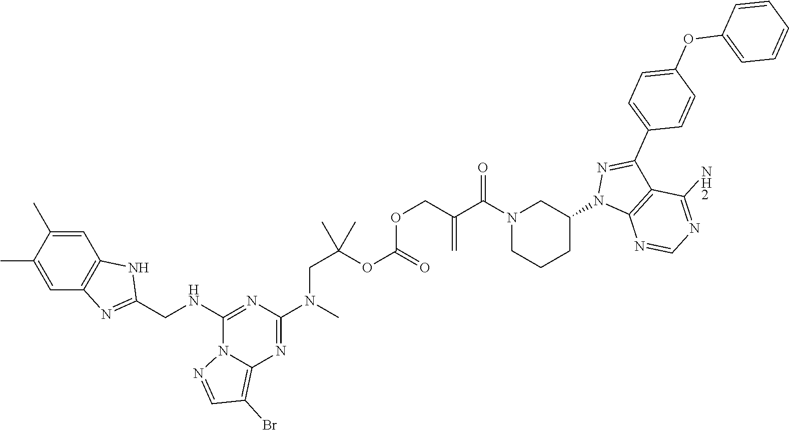 [SMILES: C=C(COC(=O)OC(C)(C)CN(C)c1nc(NCc2nc3cc(C)c(C)cc3[nH]2)n2ncc(Br)c2n1)C(=O)N1CCC[C@@H](n2nc(-c3ccc(Oc4ccccc4)cc3)c3c(N)ncnc32)C1] |
| II-118 | 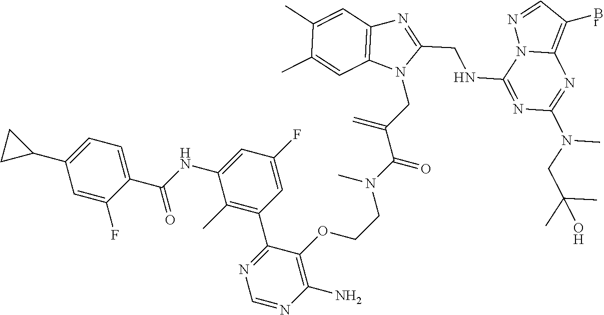 [SMILES: C=C(Cn1c(CNc2nc(N(C)CC(C)(C)O)nc3c(Br)cnn23)nc2cc(C)c(C)cc21)C(=O)N(C)CCOc1c(N)ncnc1-c1cc(F)cc(NC(=O)c2ccc(C3CC3)cc2F)c1C] |
| II-119 | 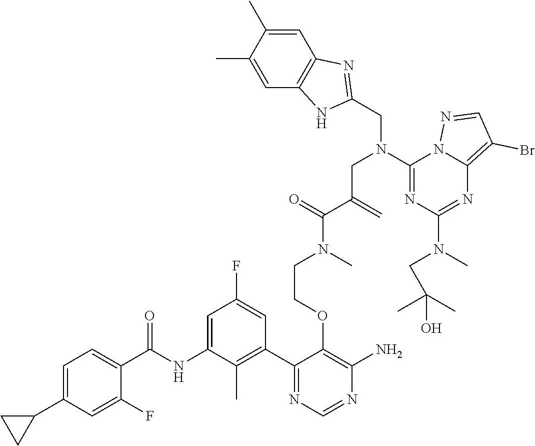 [SMILES: C=C(CN(Cc1nc2cc(C)c(C)cc2[nH]1)c1nc(N(C)CC(C)(C)O)nc2c(Br)cnn12)C(=O)N(C)CCOc1c(N)ncnc1-c1cc(F)cc(NC(=O)c2ccc(C3CC3)cc2F)c1C] |
| II-120 | 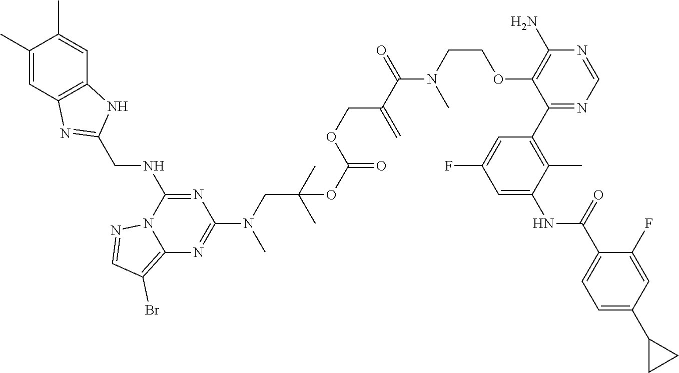 [SMILES: C=C(COC(=O)OC(C)(C)CN(C)c1nc(NCc2nc3cc(C)c(C)cc3[nH]2)n2ncc(Br)c2n1)C(=O)N(C)CCOc1c(N)ncnc1-c1cc(F)cc(NC(=O)c2ccc(C3CC3)cc2F)c1C] |
| II-121 | 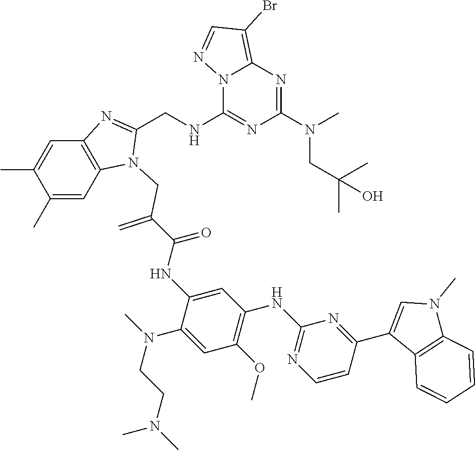 [SMILES: C=C(Cn1c(CNc2nc(N(C)CC(C)(C)O)nc3c(Br)cnn23)nc2cc(C)c(C)cc21)C(=O)Nc1cc(Nc2nccc(-c3cn(C)c4ccccc34)n2)c(OC)cc1N(C)CCN(C)C] |
| II-122 | 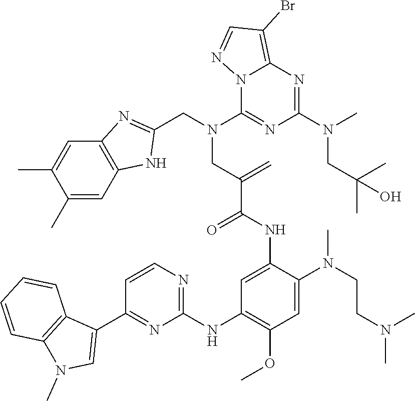 [SMILES: C=C(CN(Cc1nc2cc(C)c(C)cc2[nH]1)c1nc(N(C)CC(C)(C)O)nc2c(Br)cnn12)C(=O)Nc1cc(Nc2nccc(-c3cn(C)c4ccccc34)n2)c(OC)cc1N(C)CCN(C)C] |
| II-123 | 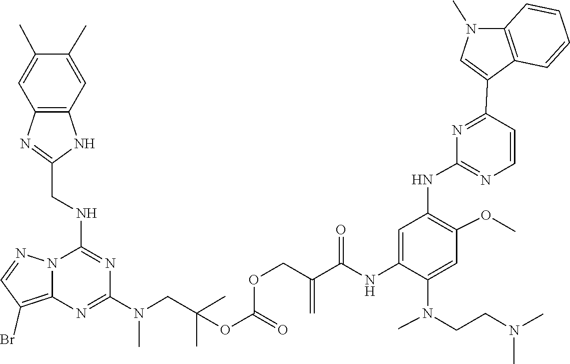 [SMILES: C=C(COC(=O)OC(C)(C)CN(C)c1nc(NCc2nc3cc(C)c(C)cc3[nH]2)n2ncc(Br)c2n1)C(=O)Nc1cc(Nc2nccc(-c3cn(C)c4ccccc34)n2)c(OC)cc1N(C)CCN(C)C] |
| II-124 | 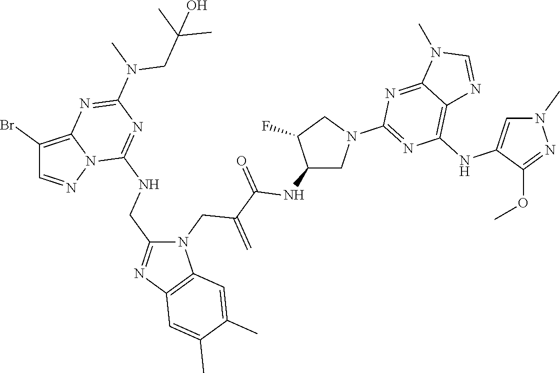 [SMILES: C=C(Cn1c(CNc2nc(N(C)CC(C)(C)O)nc3c(Br)cnn23)nc2cc(C)c(C)cc21)C(=O)N[C@@H]1CN(c2nc(Nc3cn(C)nc3OC)c3ncn(C)c3n2)C[C@H]1F] |
| II-125 | 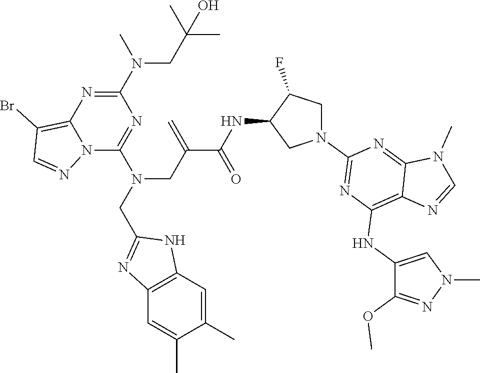 [SMILES: C=C(CN(Cc1nc2cc(C)c(C)cc2[nH]1)c1nc(N(C)CC(C)(C)O)nc2c(Br)cnn12)C(=O)N[C@@H]1CN(c2nc(Nc3cn(C)nc3OC)c3ncn(C)c3n2)C[C@H]1F] |
| II-126 | 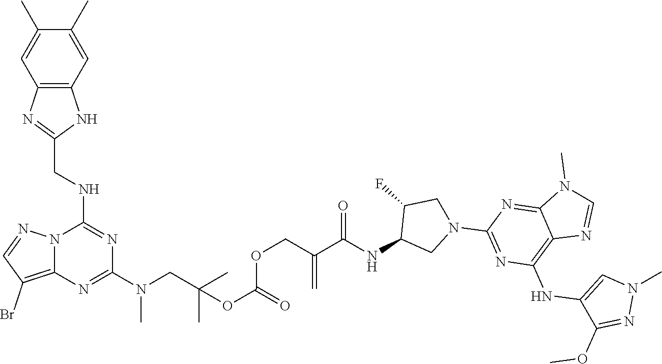 [SMILES: C=C(COC(=O)OC(C)(C)CN(C)c1nc(NCc2nc3cc(C)c(C)cc3[nH]2)n2ncc(Br)c2n1)C(=O)N[C@@H]1CN(c2nc(Nc3cn(C)nc3OC)c3ncn(C)c3n2)C[C@H]1F] |
| II-127 | 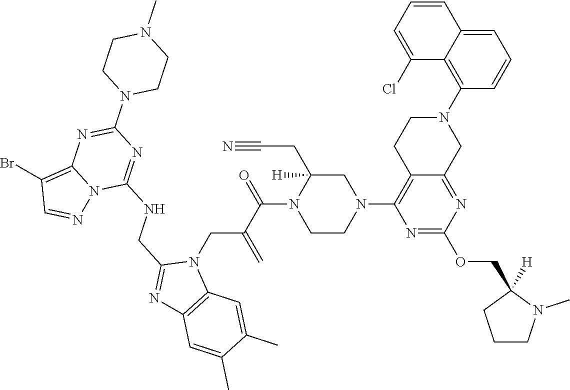 [SMILES: C=C(Cn1c(CNc2nc(N3CCN(C)CC3)nc3c(Br)cnn23)nc2cc(C)c(C)cc21)C(=O)N1CCN(c2nc(OC[C@@H]3CCCN3C)nc3c2CCN(c2cccc4cccc(Cl)c24)C3)C[C@@H]1CC#N] |
| II-128 | 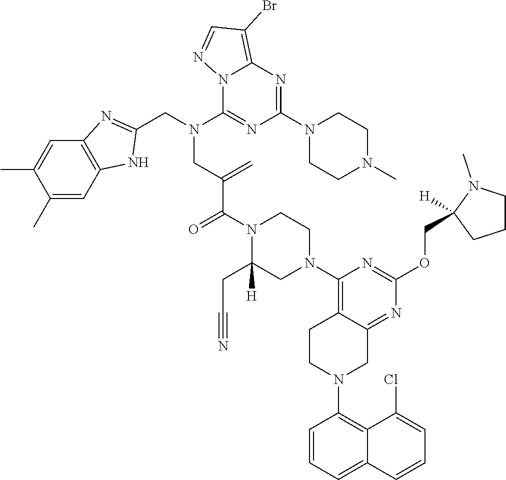 [SMILES: C=C(CN(Cc1nc2cc(C)c(C)cc2[nH]1)c1nc(N2CCN(C)CC2)nc2c(Br)cnn12)C(=O)N1CCN(c2nc(OC[C@@H]3CCCN3C)nc3c2CCN(c2cccc4cccc(Cl)c24)C3)C[C@@H]1CC#N] |
| II-129 | 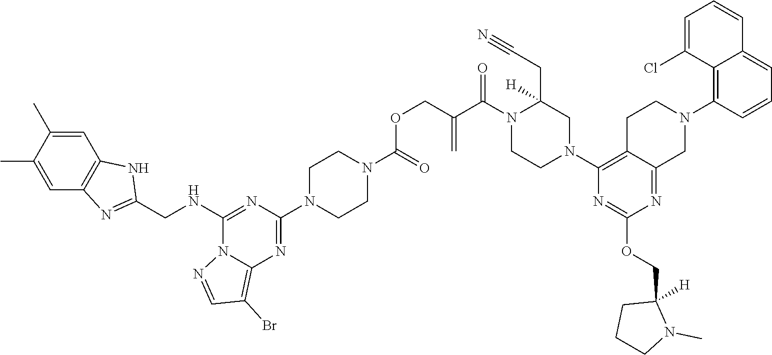 [SMILES: C=C(COC(=O)N1CCN(c2nc(NCc3nc4cc(C)c(C)cc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| II-130 | 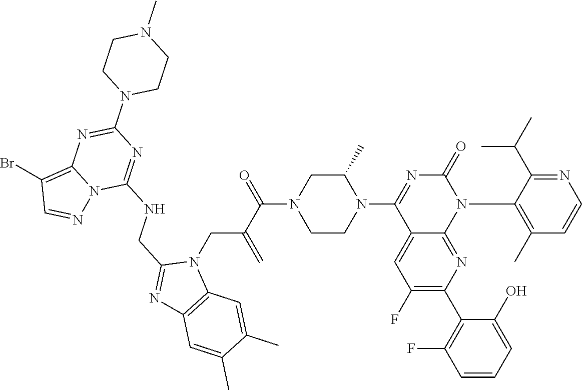 [SMILES: C=C(Cn1c(CNc2nc(N3CCN(C)CC3)nc3c(Br)cnn23)nc2cc(C)c(C)cc21)C(=O)N1CCN(c2nc(=O)n(-c3c(C)ccnc3C(C)C)c3nc(-c4c(O)cccc4F)c(F)cc23)[C@@H](C)C1] |
| II-131 | 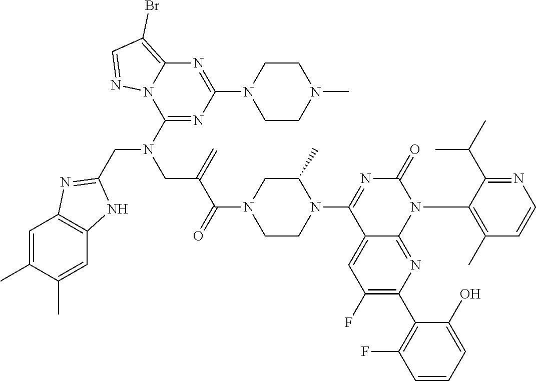 [SMILES: C=C(CN(Cc1nc2cc(C)c(C)cc2[nH]1)c1nc(N2CCN(C)CC2)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-132 | 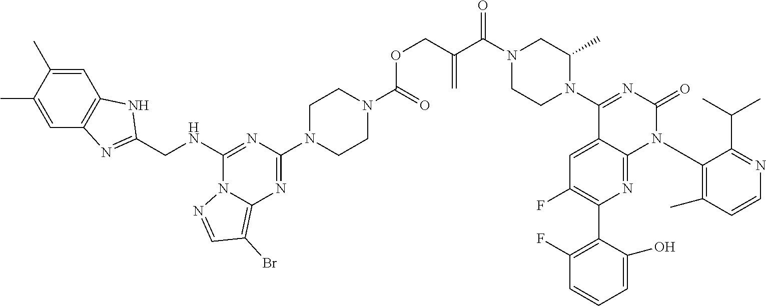 [SMILES: C=C(COC(=O)N1CCN(c2nc(NCc3nc4cc(C)c(C)cc4[nH]3)n3ncc(Br)c3n2)CC1)C(=O)N1CCN(c2nc(=O)n(-c3c(C)ccnc3C(C)C)c3nc(-c4c(O)cccc4F)c(F)cc23)[C@@H](C)C1] |
| II-133 | 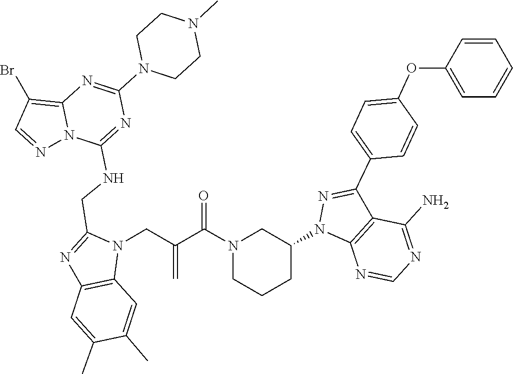 [SMILES: C=C(Cn1c(CNc2nc(N3CCN(C)CC3)nc3c(Br)cnn23)nc2cc(C)c(C)cc21)C(=O)N1CCC[C@@H](n2nc(-c3ccc(Oc4ccccc4)cc3)c3c(N)ncnc32)C1] |
| II-134 | 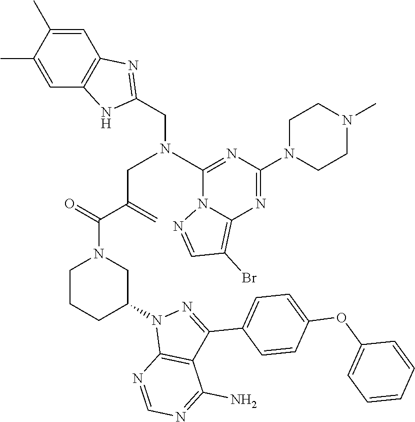 [SMILES: C=C(CN(Cc1nc2cc(C)c(C)cc2[nH]1)c1nc(N2CCN(C)CC2)nc2c(Br)cnn12)C(=O)N1CCC[C@@H](n2nc(-c3ccc(Oc4ccccc4)cc3)c3c(N)ncnc32)C1] |
| II-135 | 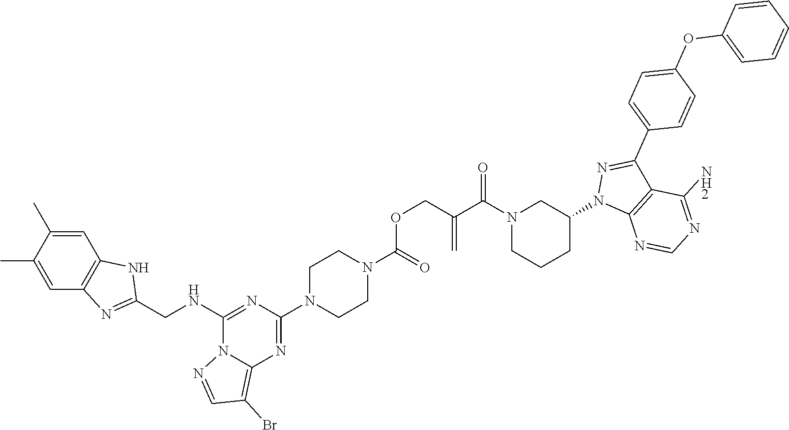 [SMILES: C=C(COC(=O)N1CCN(c2nc(NCc3nc4cc(C)c(C)cc4[nH]3)n3ncc(Br)c3n2)CC1)C(=O)N1CCC[C@@H](n2nc(-c3ccc(Oc4ccccc4)cc3)c3c(N)ncnc32)C1] |
| II-136 | 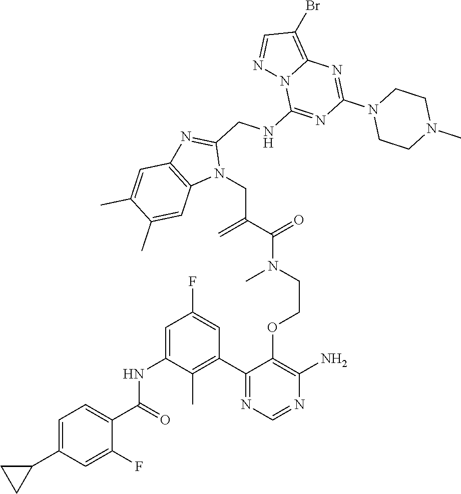 [SMILES: C=C(Cn1c(CNc2nc(N3CCN(C)CC3)nc3c(Br)cnn23)nc2cc(C)c(C)cc21)C(=O)N(C)CCOc1c(N)ncnc1-c1cc(F)cc(NC(=O)c2ccc(C3CC3)cc2F)c1C] |
| II-137 | 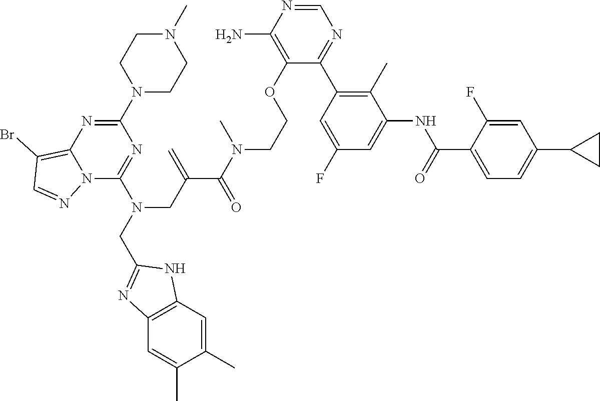 [SMILES: C=C(CN(Cc1nc2cc(C)c(C)cc2[nH]1)c1nc(N2CCN(C)CC2)nc2c(Br)cnn12)C(=O)N(C)CCOc1c(N)ncnc1-c1cc(F)cc(NC(=O)c2ccc(C3CC3)cc2F)c1C] |
| II-138 | 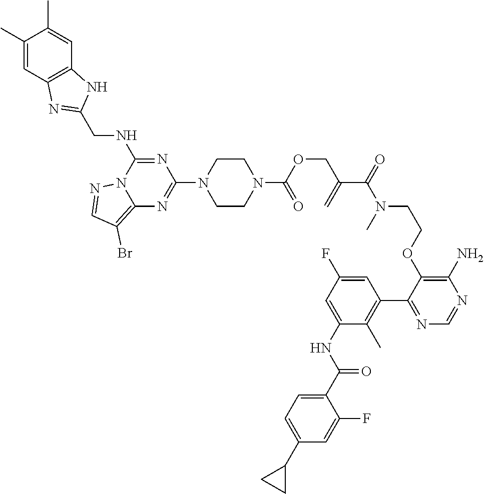 [SMILES: C=C(COC(=O)N1CCN(c2nc(NCc3nc4cc(C)c(C)cc4[nH]3)n3ncc(Br)c3n2)CC1)C(=O)N(C)CCOc1c(N)ncnc1-c1cc(F)cc(NC(=O)c2ccc(C3CC3)cc2F)c1C] |
| II-139 | 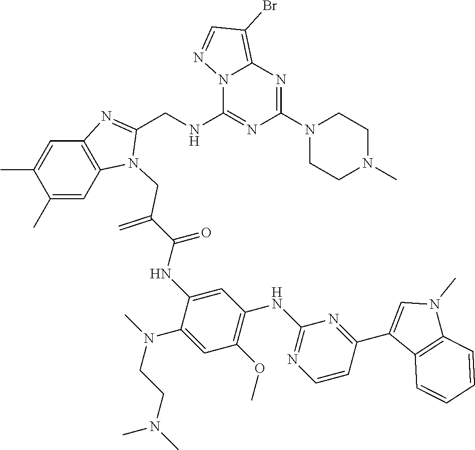 [SMILES: C=C(Cn1c(CNc2nc(N3CCN(C)CC3)nc3c(Br)cnn23)nc2cc(C)c(C)cc21)C(=O)Nc1cc(Nc2nccc(-c3cn(C)c4ccccc34)n2)c(OC)cc1N(C)CCN(C)C] |
| II-140 | 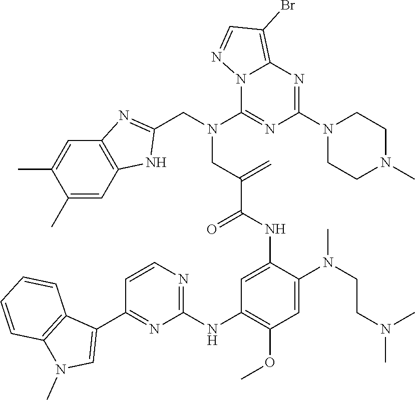 [SMILES: C=C(CN(Cc1nc2cc(C)c(C)cc2[nH]1)c1nc(N2CCN(C)CC2)nc2c(Br)cnn12)C(=O)Nc1cc(Nc2nccc(-c3cn(C)c4ccccc34)n2)c(OC)cc1N(C)CCN(C)C] |
| II-141 | 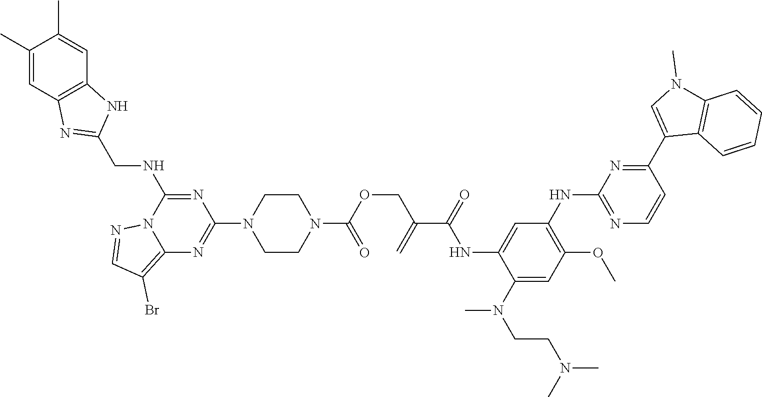 [SMILES: C=C(COC(=O)N1CCN(c2nc(NCc3nc4cc(C)c(C)cc4[nH]3)n3ncc(Br)c3n2)CC1)C(=O)Nc1cc(Nc2nccc(-c3cn(C)c4ccccc34)n2)c(OC)cc1N(C)CCN(C)C] |
| II-142 | 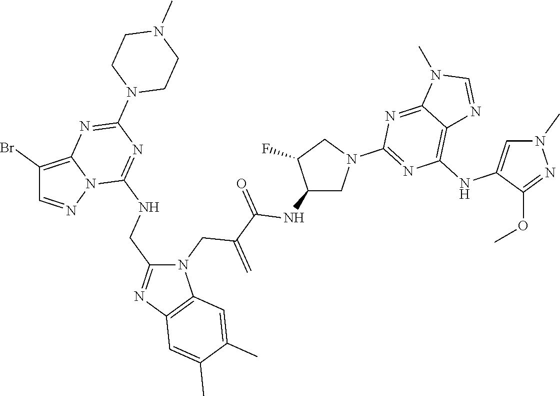 [SMILES: C=C(Cn1c(CNc2nc(N3CCN(C)CC3)nc3c(Br)cnn23)nc2cc(C)c(C)cc21)C(=O)N[C@@H]1CN(c2nc(Nc3cn(C)nc3OC)c3ncn(C)c3n2)C[C@H]1F] |
| II-143 | 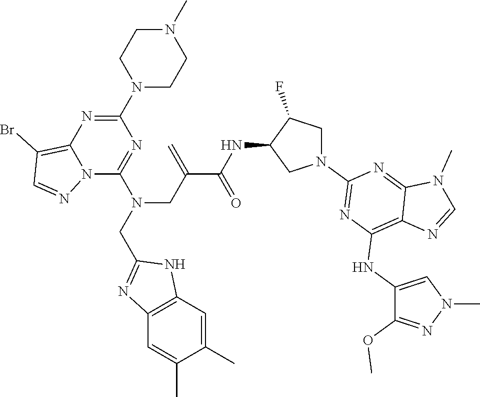 [SMILES: C=C(CN(Cc1nc2cc(C)c(C)cc2[nH]1)c1nc(N2CCN(C)CC2)nc2c(Br)cnn12)C(=O)N[C@@H]1CN(c2nc(Nc3cn(C)nc3OC)c3ncn(C)c3n2)C[C@H]1F] |
| II-144 | 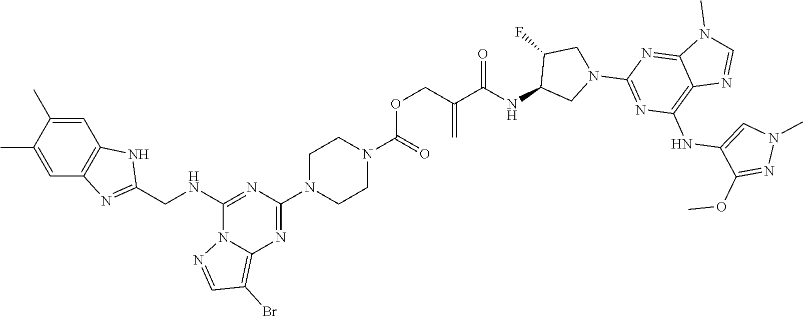 |
| II-145 | 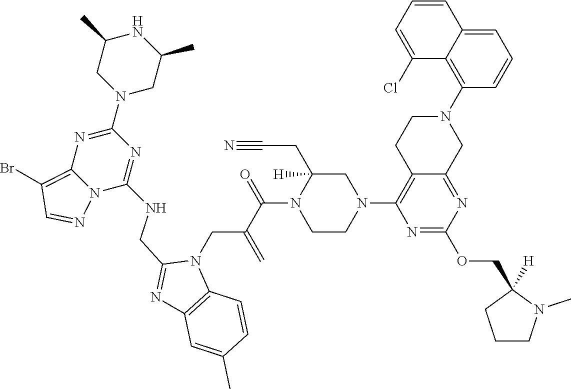 [SMILES: C=C(Cn1c(CNc2nc(N3C[C@@H](C)N[C@@H](C)C3)nc3c(Br)cnn23)nc2cc(C)ccc21)C(=O)N1CCN(c2nc(OC[C@@H]3CCCN3C)nc3c2CCN(c2cccc4cccc(Cl)c24)C3)C[C@@H]1CC#N] |
| II-146 | 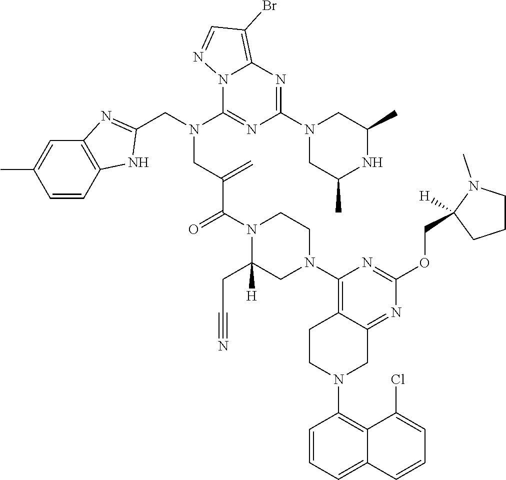 [SMILES: C=C(CN(Cc1nc2cc(C)ccc2[nH]1)c1nc(N2C[C@@H](C)N[C@@H](C)C2)nc2c(Br)cnn12)C(=O)N1CCN(c2nc(OC[C@@H]3CCCN3C)nc3c2CCN(c2cccc4cccc(Cl)c24)C3)C[C@@H]1CC#N] |
| II-147 | 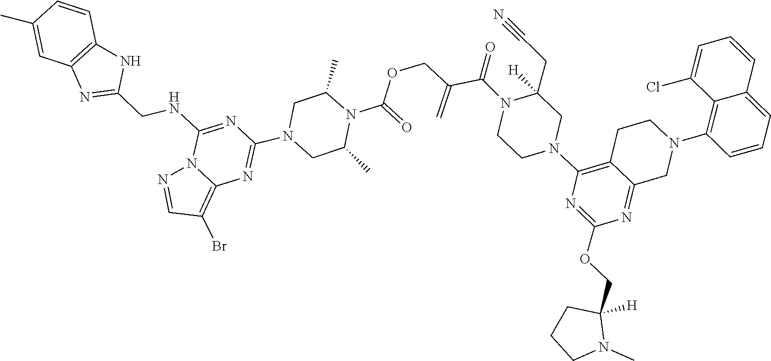 [SMILES: C=C(COC(=O)N1[C@H](C)CN(c2nc(NCc3nc4cc(C)ccc4[nH]3)n3ncc(Br)c3n2)C[C@@H]1C)C(=O)N1CCN(c2nc(OC[C@@H]3CCCN3C)nc3c2CCN(c2cccc4cccc(Cl)c24)C3)C[C@@H]1CC#N] |
| II-148 | 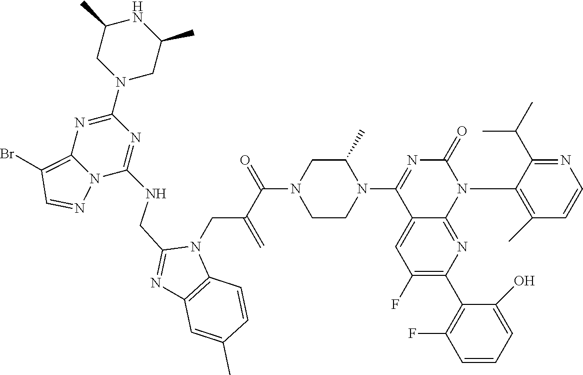 [SMILES: C=C(Cn1c(CNc2nc(N3C[C@@H](C)N[C@@H](C)C3)nc3c(Br)cnn23)nc2cc(C)ccc21)C(=O)N1CCN(c2nc(=O)n(-c3c(C)ccnc3C(C)C)c3nc(-c4c(O)cccc4F)c(F)cc23)[C@@H](C)C1] |
| II-149 | 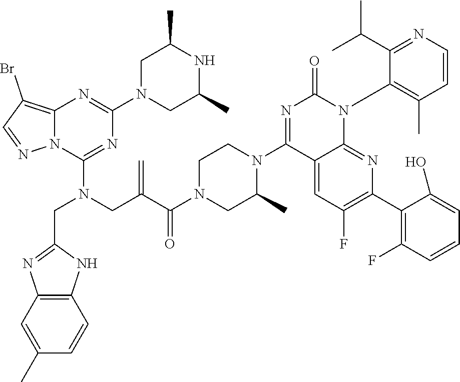 [SMILES: C=C(CN(Cc1nc2cc(C)ccc2[nH]1)c1nc(N2C[C@@H](C)N[C@@H](C)C2)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-150 | 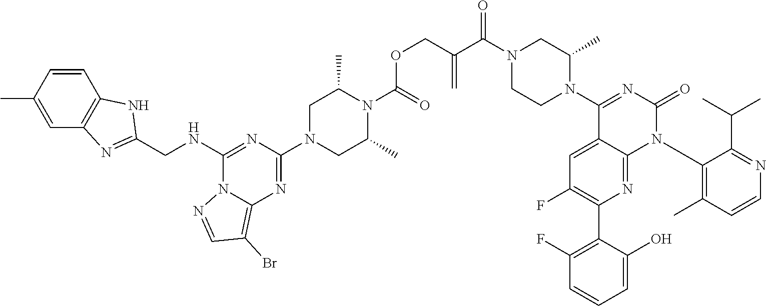 [SMILES: C=C(COC(=O)N1[C@H](C)CN(c2nc(NCc3nc4cc(C)ccc4[nH]3)n3ncc(Br)c3n2)C[C@@H]1C)C(=O)N1CCN(c2nc(=O)n(-c3c(C)ccnc3C(C)C)c3nc(-c4c(O)cccc4F)c(F)cc23)[C@@H](C)C1] |
| II-151 | 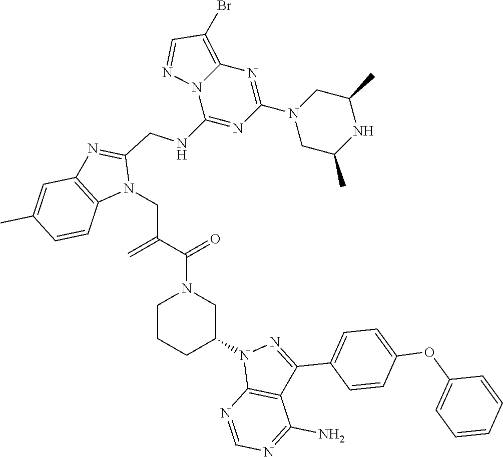 [SMILES: C=C(Cn1c(CNc2nc(N3C[C@@H](C)N[C@@H](C)C3)nc3c(Br)cnn23)nc2cc(C)ccc21)C(=O)N1CCC[C@@H](n2nc(-c3ccc(Oc4ccccc4)cc3)c3c(N)ncnc32)C1] |
| II-152 | 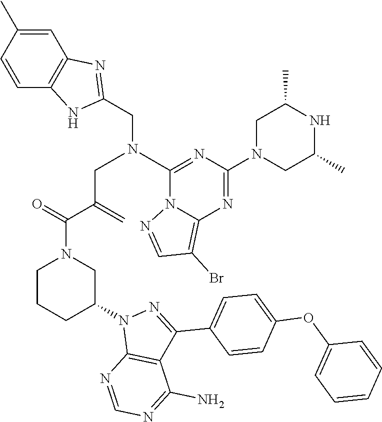 [SMILES: C=C(CN(Cc1nc2cc(C)ccc2[nH]1)c1nc(N2C[C@@H](C)N[C@@H](C)C2)nc2c(Br)cnn12)C(=O)N1CCC[C@@H](n2nc(-c3ccc(Oc4ccccc4)cc3)c3c(N)ncnc32)C1] |
| II-153 | 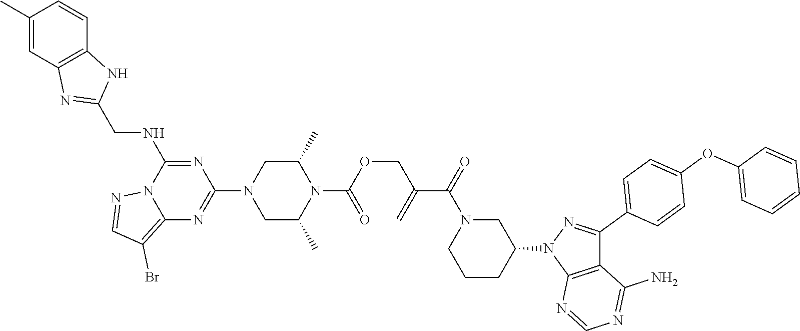 [SMILES: C=C(COC(=O)N1[C@H](C)CN(c2nc(NCc3nc4cc(C)ccc4[nH]3)n3ncc(Br)c3n2)C[C@@H]1C)C(=O)N1CCC[C@@H](n2nc(-c3ccc(Oc4ccccc4)cc3)c3c(N)ncnc32)C1] |
| II-154 | 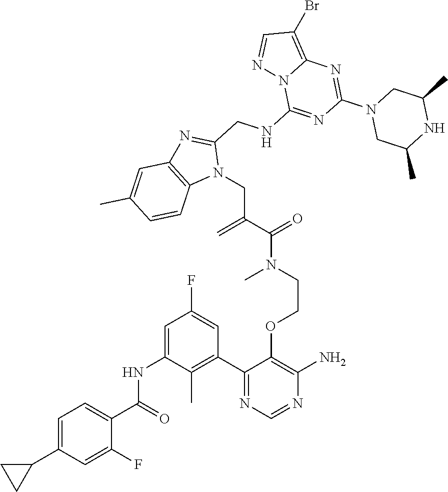 [SMILES: C=C(Cn1c(CNc2nc(N3C[C@@H](C)N[C@@H](C)C3)nc3c(Br)cnn23)nc2cc(C)ccc21)C(=O)N(C)CCOc1c(N)ncnc1-c1cc(F)cc(NC(=O)c2ccc(C3CC3)cc2F)c1C] |
| II-155 | 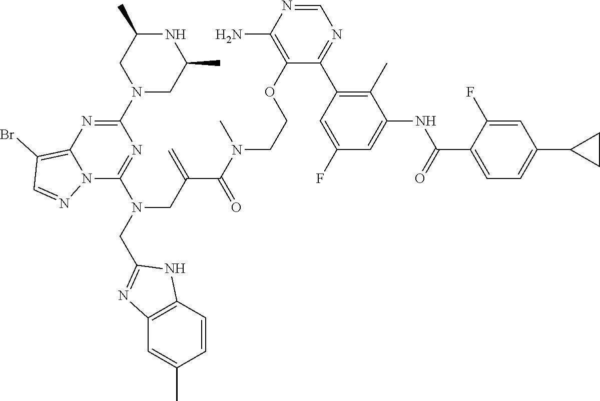 [SMILES: C=C(CN(Cc1nc2cc(C)ccc2[nH]1)c1nc(N2C[C@@H](C)N[C@@H](C)C2)nc2c(Br)cnn12)C(=O)N(C)CCOc1c(N)ncnc1-c1cc(F)cc(NC(=O)c2ccc(C3CC3)cc2F)c1C] |
| II-156 | 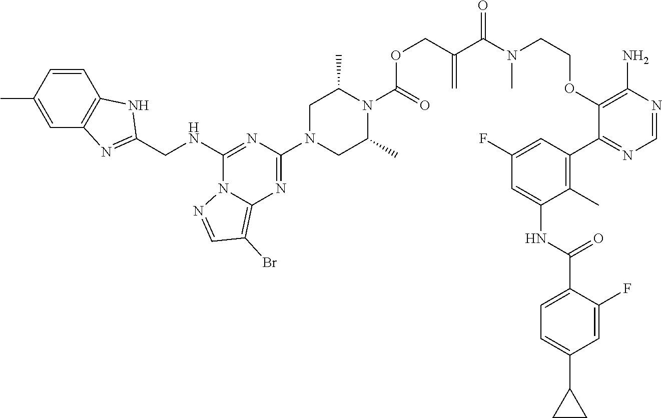 [SMILES: C=C(COC(=O)N1[C@H](C)CN(c2nc(NCc3nc4cc(C)ccc4[nH]3)n3ncc(Br)c3n2)C[C@@H]1C)C(=O)N(C)CCOc1c(N)ncnc1-c1cc(F)cc(NC(=O)c2ccc(C3CC3)cc2F)c1C] |
| II-157 | 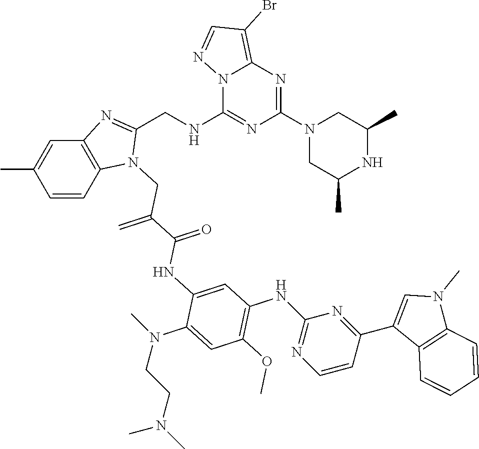 [SMILES: C=C(Cn1c(CNc2nc(N3C[C@@H](C)N[C@@H](C)C3)nc3c(Br)cnn23)nc2cc(C)ccc21)C(=O)Nc1cc(Nc2nccc(-c3cn(C)c4ccccc34)n2)c(OC)cc1N(C)CCN(C)C] |
| II-158 | 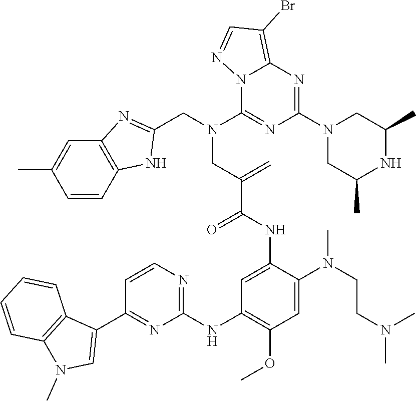 [SMILES: C=C(CN(Cc1nc2cc(C)ccc2[nH]1)c1nc(N2C[C@@H](C)N[C@@H](C)C2)nc2c(Br)cnn12)C(=O)Nc1cc(Nc2nccc(-c3cn(C)c4ccccc34)n2)c(OC)cc1N(C)CCN(C)C] |
| II-159 | 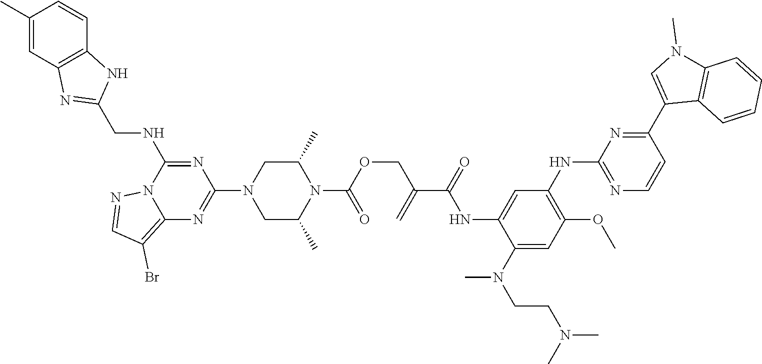 [SMILES: C=C(COC(=O)N1[C@H](C)CN(c2nc(NCc3nc4cc(C)ccc4[nH]3)n3ncc(Br)c3n2)C[C@@H]1C)C(=O)Nc1cc(Nc2nccc(-c3cn(C)c4ccccc34)n2)c(OC)cc1N(C)CCN(C)C] |
| II-160 | 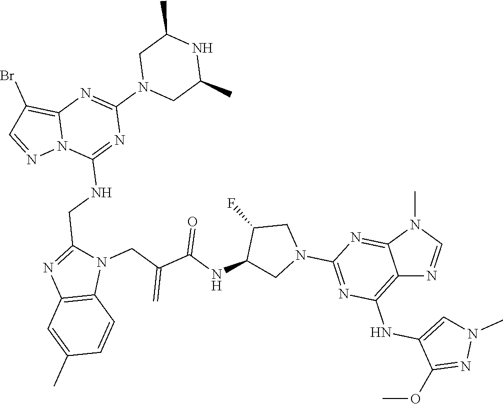 [SMILES: C=C(Cn1c(CNc2nc(N3C[C@@H](C)N[C@@H](C)C3)nc3c(Br)cnn23)nc2cc(C)ccc21)C(=O)N[C@@H]1CN(c2nc(Nc3cn(C)nc3OC)c3ncn(C)c3n2)C[C@H]1F] |
| II-161 | 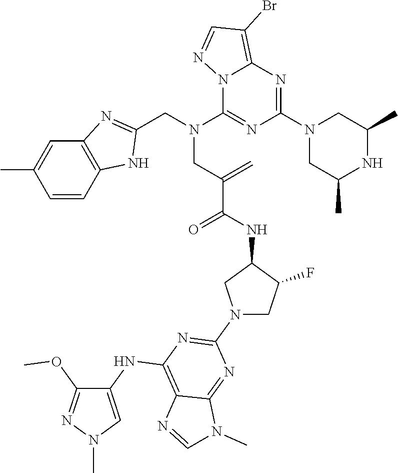 |
| II-162 | 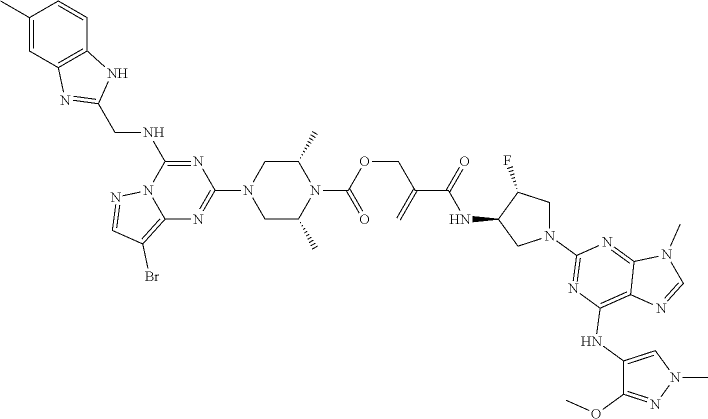 |
| II-163 | 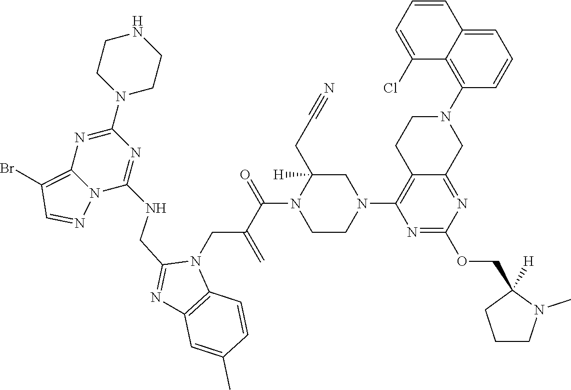 [SMILES: C=C(Cn1c(CNc2nc(N3CCNCC3)nc3c(Br)cnn23)nc2cc(C)ccc21)C(=O)N1CCN(c2nc(OC[C@@H]3CCCN3C)nc3c2CCN(c2cccc4cccc(Cl)c24)C3)C[C@@H]1CC#N] |
| II-164 | 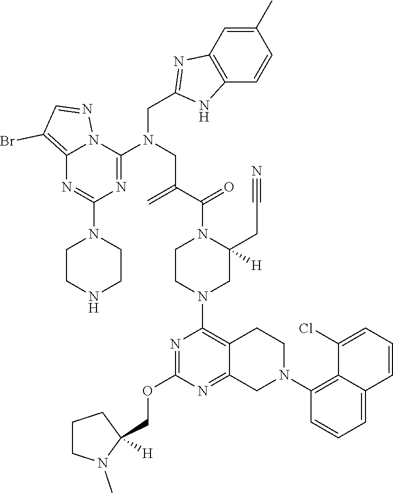 [SMILES: C=C(CN(Cc1nc2cc(C)ccc2[nH]1)c1nc(N2CCNCC2)nc2c(Br)cnn12)C(=O)N1CCN(c2nc(OC[C@@H]3CCCN3C)nc3c2CCN(c2cccc4cccc(Cl)c24)C3)C[C@@H]1CC#N] |
| II-165 | 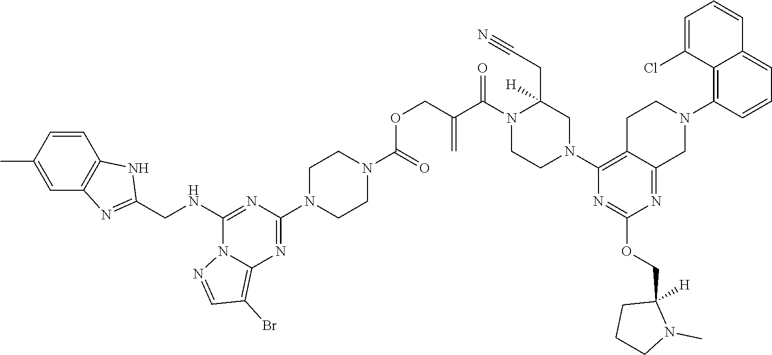 [SMILES: C=C(COC(=O)N1CCN(c2nc(NCc3nc4cc(C)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| II-166 | 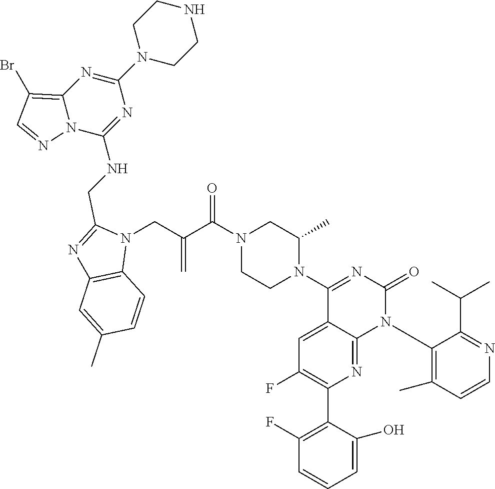 [SMILES: C=C(Cn1c(CNc2nc(N3CCNCC3)nc3c(Br)cnn23)nc2cc(C)ccc21)C(=O)N1CCN(c2nc(=O)n(-c3c(C)ccnc3C(C)C)c3nc(-c4c(O)cccc4F)c(F)cc23)[C@@H](C)C1] |
| II-167 | 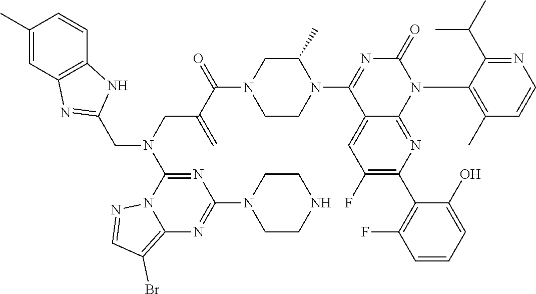 [SMILES: C=C(CN(Cc1nc2cc(C)ccc2[nH]1)c1nc(N2CCNCC2)nc2c(Br)cnn12)C(=O)N1CCN(c2nc(=O)n(-c3c(C)ccnc3C(C)C)c3nc(-c4c(O)cccc4F)c(F)cc23)[C@@H](C)C1] |
| II-168 | 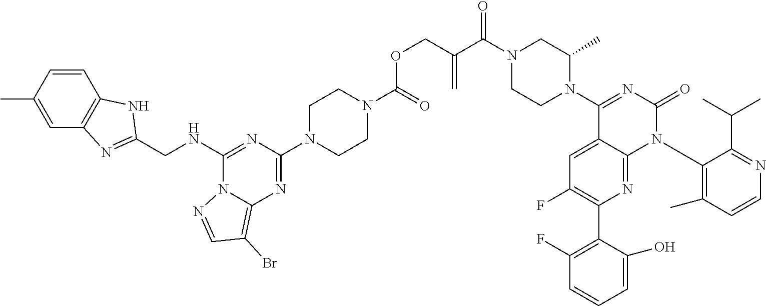 [SMILES: C=C(COC(=O)N1CCN(c2nc(NCc3nc4cc(C)ccc4[nH]3)n3ncc(Br)c3n2)CC1)C(=O)N1CCN(c2nc(=O)n(-c3c(C)ccnc3C(C)C)c3nc(-c4c(O)cccc4F)c(F)cc23)[C@@H](C)C1] |
| II-169 | 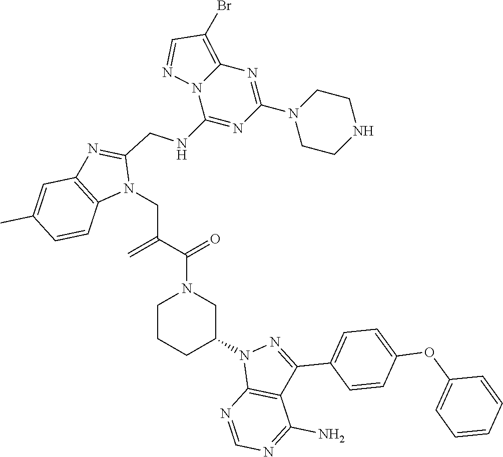 [SMILES: C=C(Cn1c(CNc2nc(N3CCNCC3)nc3c(Br)cnn23)nc2cc(C)ccc21)C(=O)N1CCC[C@@H](n2nc(-c3ccc(Oc4ccccc4)cc3)c3c(N)ncnc32)C1] |
| II-170 | 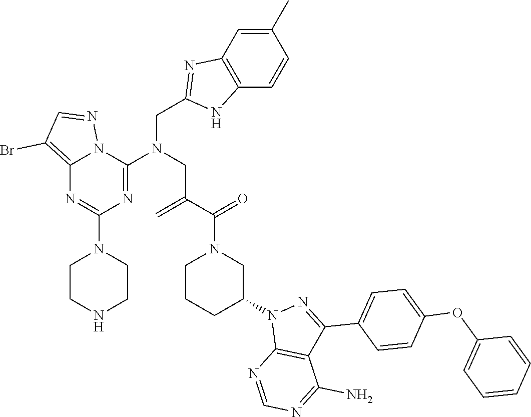 [SMILES: C=C(CN(Cc1nc2cc(C)ccc2[nH]1)c1nc(N2CCNCC2)nc2c(Br)cnn12)C(=O)N1CCC[C@@H](n2nc(-c3ccc(Oc4ccccc4)cc3)c3c(N)ncnc32)C1] |
| II-171 | 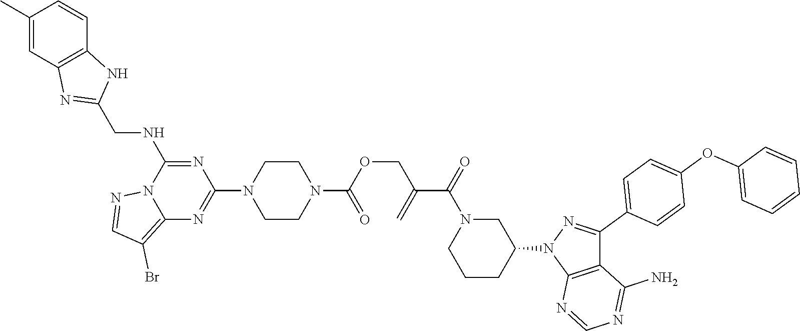 [SMILES: C=C(COC(=O)N1CCN(c2nc(NCc3nc4cc(C)ccc4[nH]3)n3ncc(Br)c3n2)CC1)C(=O)N1CCC[C@@H](n2nc(-c3ccc(Oc4ccccc4)cc3)c3c(N)ncnc32)C1] |
| II-172 | 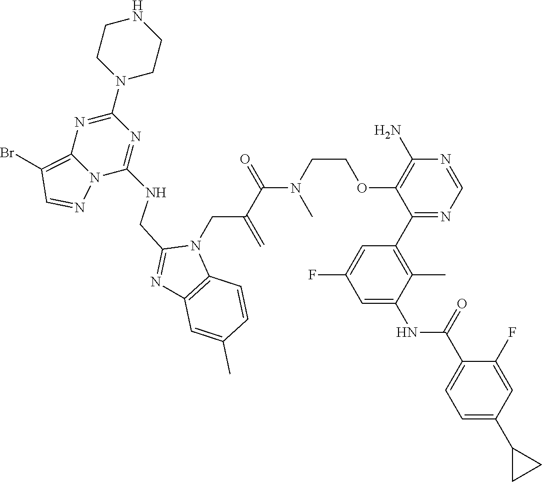 [SMILES: C=C(Cn1c(CNc2nc(N3CCNCC3)nc3c(Br)cnn23)nc2cc(C)ccc21)C(=O)N(C)CCOc1c(N)ncnc1-c1cc(F)cc(NC(=O)c2ccc(C3CC3)cc2F)c1C] |
| II-173 | 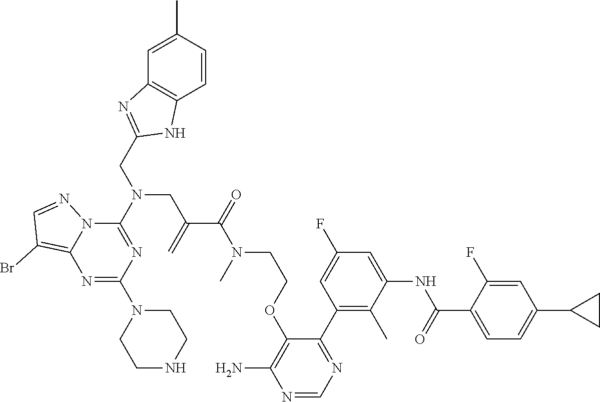 [SMILES: C=C(CN(Cc1nc2cc(C)ccc2[nH]1)c1nc(N2CCNCC2)nc2c(Br)cnn12)C(=O)N(C)CCOc1c(N)ncnc1-c1cc(F)cc(NC(=O)c2ccc(C3CC3)cc2F)c1C] |
| II-174 | 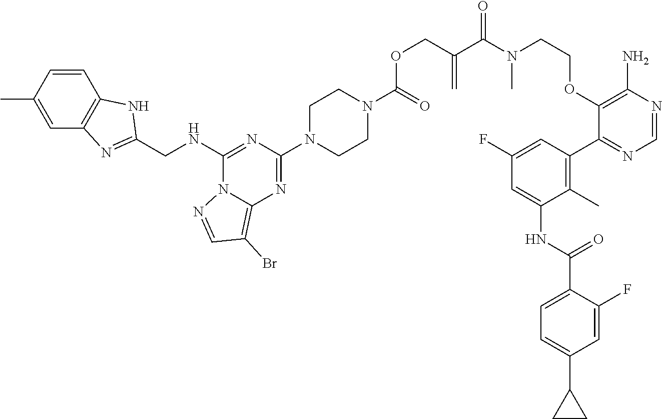 [SMILES: C=C(COC(=O)N1CCN(c2nc(NCc3nc4cc(C)ccc4[nH]3)n3ncc(Br)c3n2)CC1)C(=O)N(C)CCOc1c(N)ncnc1-c1cc(F)cc(NC(=O)c2ccc(C3CC3)cc2F)c1C] |
| II-175 | 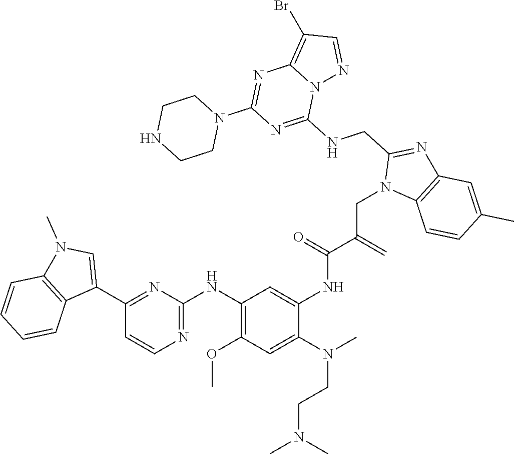 [SMILES: C=C(Cn1c(CNc2nc(N3CCNCC3)nc3c(Br)cnn23)nc2cc(C)ccc21)C(=O)Nc1cc(Nc2nccc(-c3cn(C)c4ccccc34)n2)c(OC)cc1N(C)CCN(C)C] |
| II-176 | 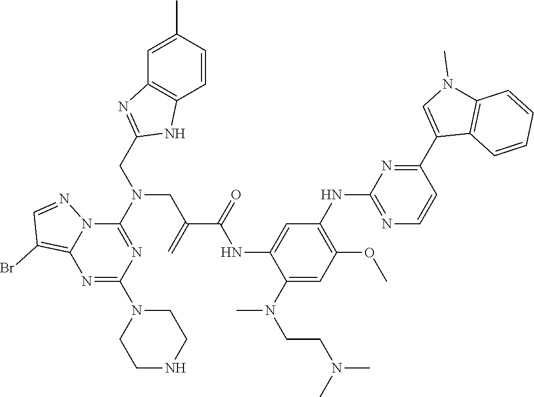 [SMILES: C=C(CN(Cc1nc2cc(C)ccc2[nH]1)c1nc(N2CCNCC2)nc2c(Br)cnn12)C(=O)Nc1cc(Nc2nccc(-c3cn(C)c4ccccc34)n2)c(OC)cc1N(C)CCN(C)C] |
| II-177 | 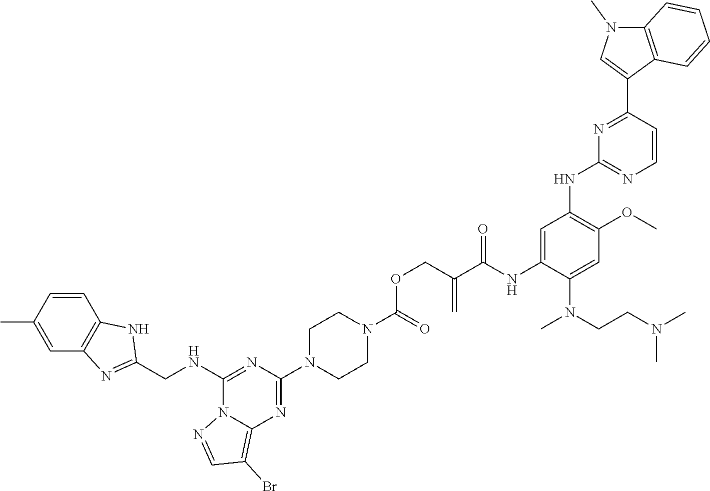 [SMILES: C=C(COC(=O)N1CCN(c2nc(NCc3nc4cc(C)ccc4[nH]3)n3ncc(Br)c3n2)CC1)C(=O)Nc1cc(Nc2nccc(-c3cn(C)c4ccccc34)n2)c(OC)cc1N(C)CCN(C)C] |
| II-178 | 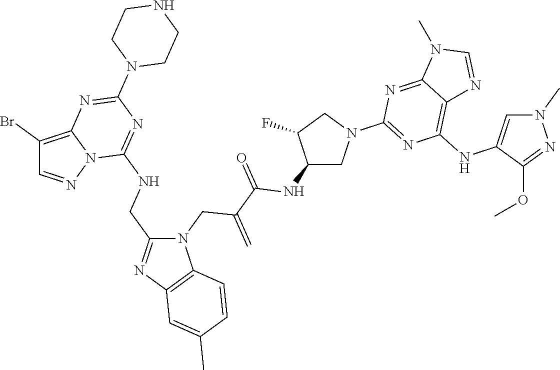 [SMILES: C=C(Cn1c(CNc2nc(N3CCNCC3)nc3c(Br)cnn23)nc2cc(C)ccc21)C(=O)N[C@@H]1CN(c2nc(Nc3cn(C)nc3OC)c3ncn(C)c3n2)C[C@H]1F] |
| II-179 | 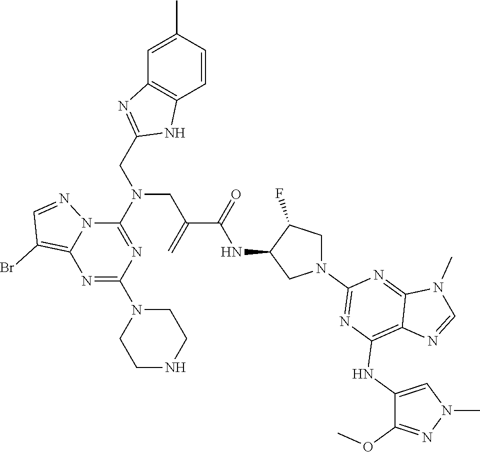 [SMILES: C=C(CN(Cc1nc2cc(C)ccc2[nH]1)c1nc(N2CCNCC2)nc2c(Br)cnn12)C(=O)N[C@@H]1CN(c2nc(Nc3cn(C)nc3OC)c3ncn(C)c3n2)C[C@H]1F] |
| II-180 | 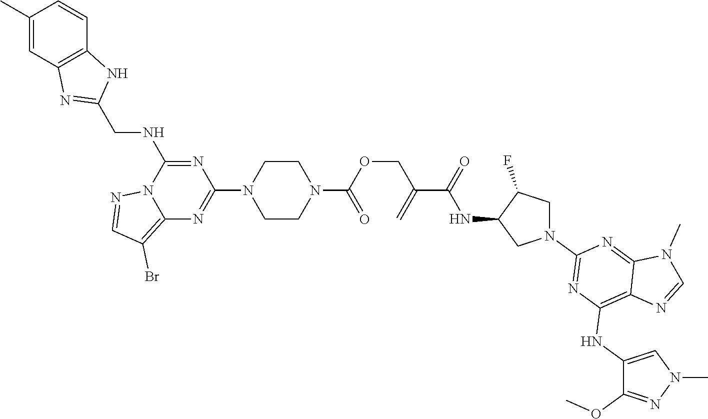 |

TABLE 2-A
Additional Compounds that Bind Cyclin K.

| No. | Chemical Structure |
|---|---|
| II-181 | 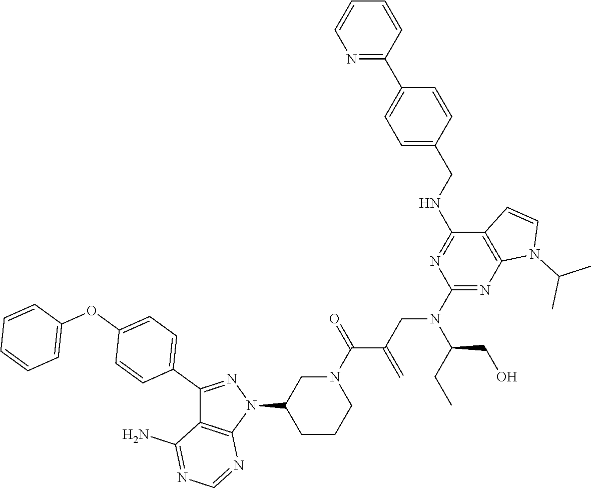 |
| II-182 | 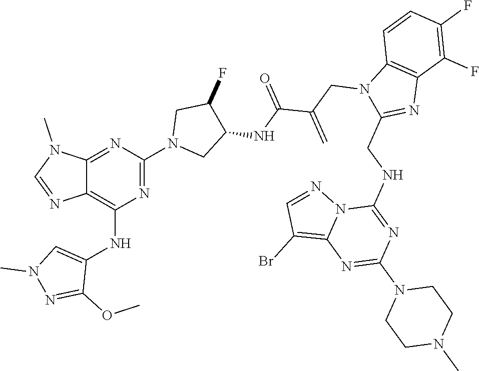 |
| II-183 | 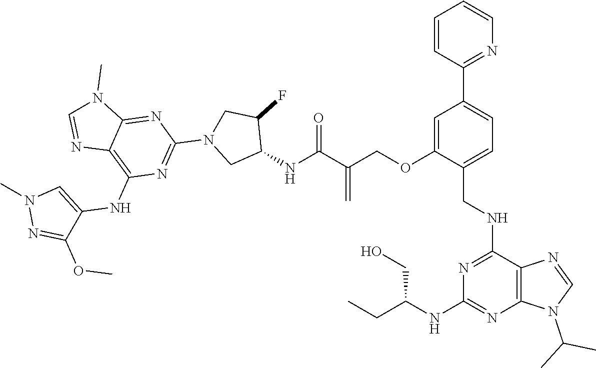 [SMILES: C=C(COc1cc(-c2ccccn2)ccc1CNc1nc(N[C@H](CC)CO)nc2c1ncn2C(C)C)C(=O)N[C@@H]1CN(c2nc(Nc3cn(C)nc3OC)c3ncn(C)c3n2)C[C@H]1F] |
| II-184 | 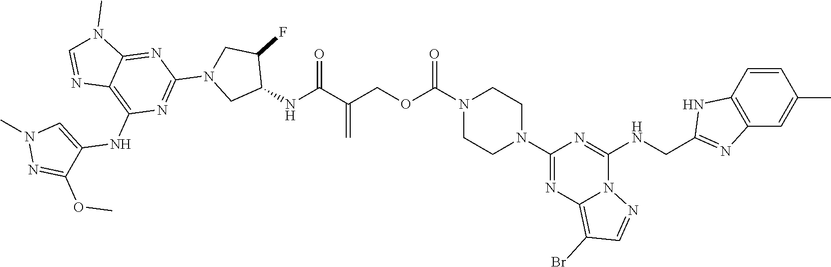 |
| II-185 | 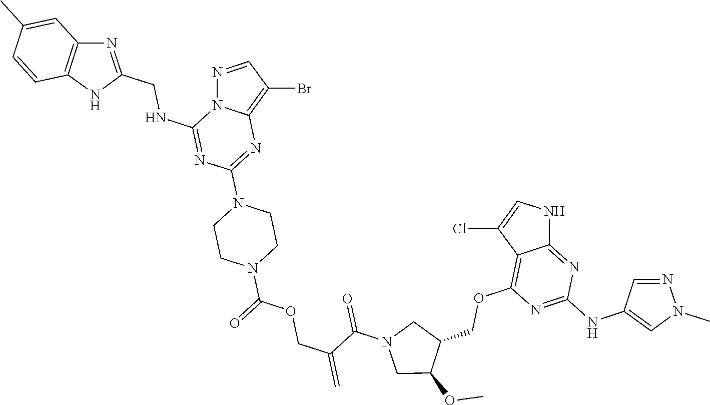 |
| II-186 | 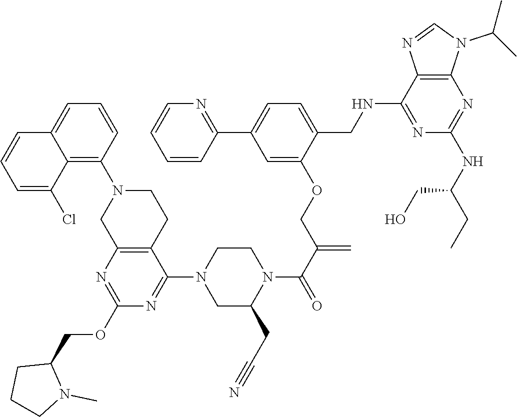 [SMILES: C=C(COc1cc(-c2ccccn2)ccc1CNc1nc(N[C@H](CC)CO)nc2c1ncn2C(C)C)C(=O)N1CCN(c2nc(OC[C@@H]3CCCN3C)nc3c2CCN(c2cccc4cccc(Cl)c24)C3)C[C@@H]1CC#N] |
| II-187 | 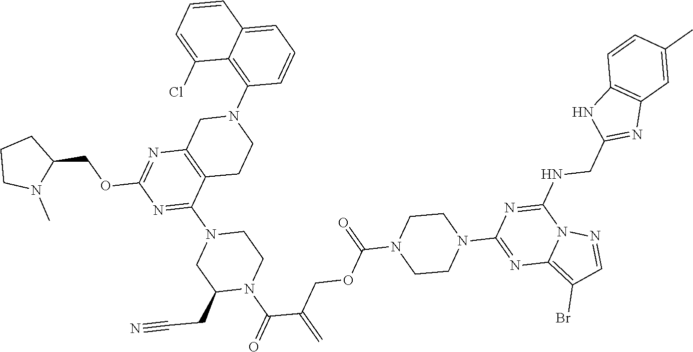 [SMILES: C=C(COC(=O)N1CCN(c2nc(NCc3nc4cc(C)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| II-188 | 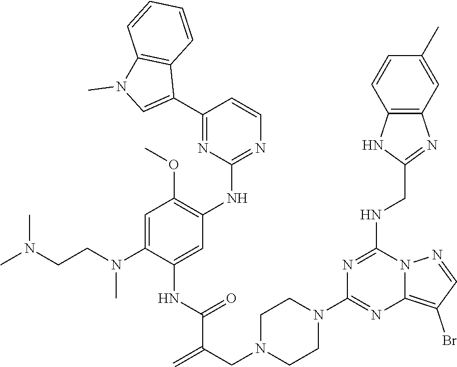 [SMILES: C=C(CN1CCN(c2nc(NCc3nc4cc(C)ccc4[nH]3)n3ncc(Br)c3n2)CC1)C(=O)Nc1cc(Nc2nccc(-c3cn(C)c4ccccc34)n2)c(OC)cc1N(C)CCN(C)C] |
| II-189 | 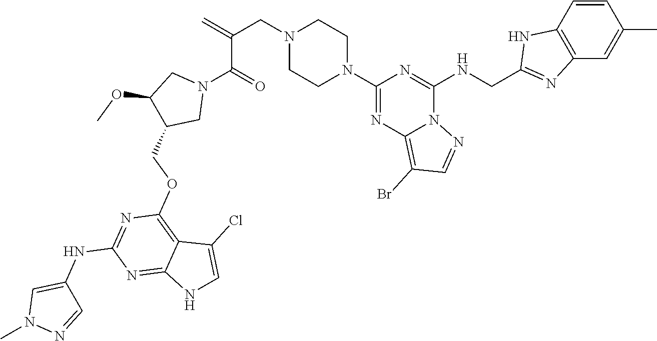 |
| II-190 | 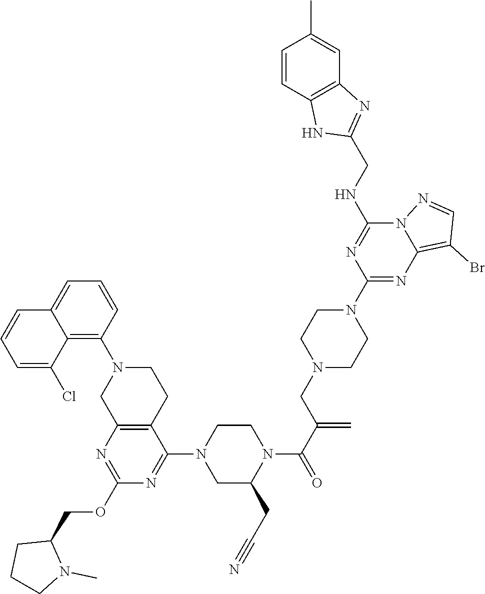 [SMILES: C=C(CN1CCN(c2nc(NCc3nc4cc(C)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| II-191 | 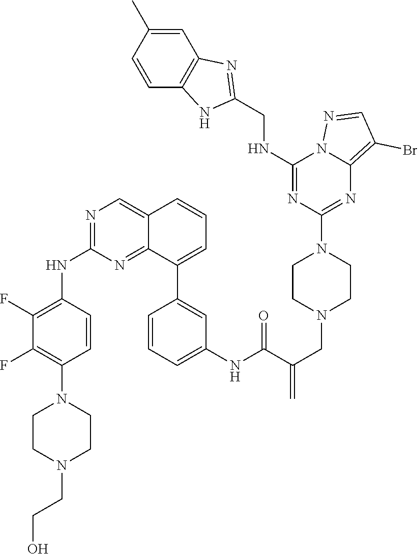 [SMILES: C=C(CN1CCN(c2nc(NCc3nc4cc(C)ccc4[nH]3)n3ncc(Br)c3n2)CC1)C(=O)Nc1cccc(-c2cccc3cnc(Nc4ccc(N5CCN(CCO)CC5)c(F)c4F)nc23)c1] |
| II-192 | 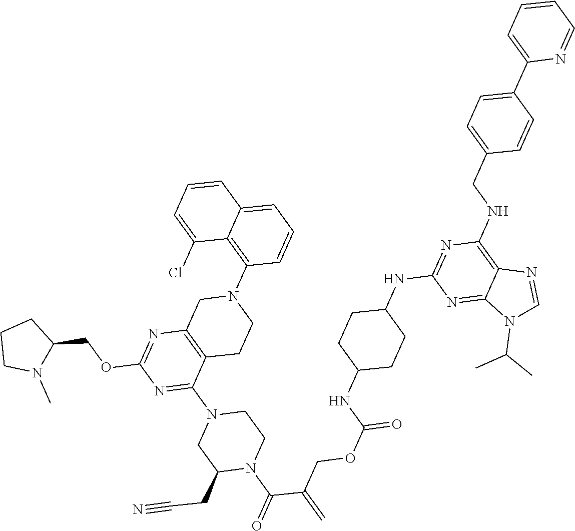 [SMILES: C=C(COC(=O)NC1CCC(Nc2nc(NCc3ccc(-c4ccccn4)cc3)c3ncn(C(C)C)c3n2)CC1)C(=O)N1CCN(c2nc(OC[C@@H]3CCCN3C)nc3c2CCN(c2cccc4cccc(Cl)c24)C3)C[C@@H]1CC#N] |
| II-193 | 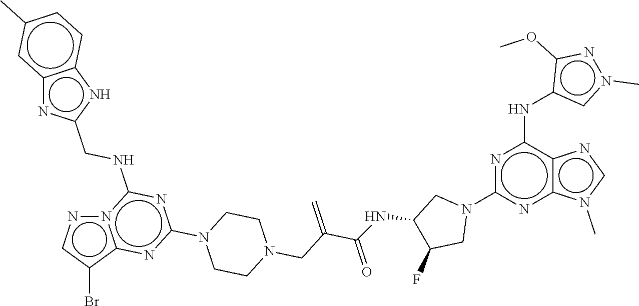 |

In certain embodiments, the compound is a compound in Table 3, or a pharmaceutically acceptable salt thereof.

TABLE 3
Additional Compounds

| No. | Chemical Structure |
|---|---|
| III-1 | 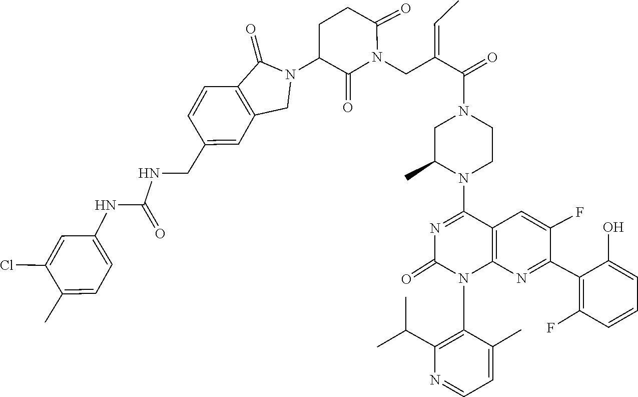 [SMILES: C/C=C(/CN1C(=O)CCC(N2Cc3cc(CNC(=O)Nc4ccc(C)c(Cl)c4)ccc3C2=O)C1=O)C(=O)N1CCN(c2nc(=O)n(-c3c(C)ccnc3C(C)C)c3nc(-c4c(O)cccc4F)c(F)cc23)[C@@H](C)C1] |
| III-2 | 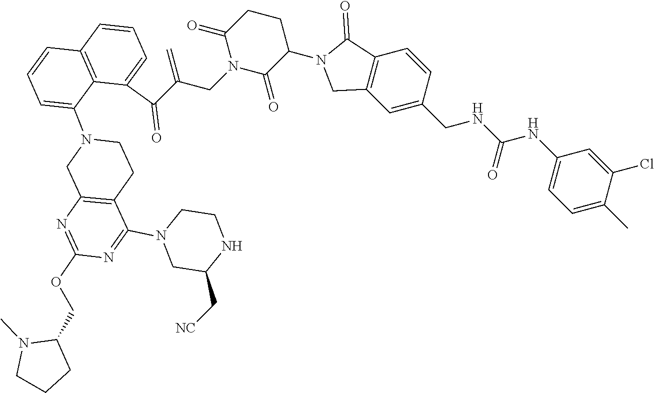 [SMILES: C=C(CN1C(=O)CCC(N2Cc3cc(CNC(=O)Nc4ccc(C)c(Cl)c4)ccc3C2=O)C1=O)C(=O)c1cccc2cccc(N3CCc4c(nc(OC[C@@H]5CCCN5C)nc4N4CCN[C@@H](CC#N)C4)C3)c12] |
| III-3 | 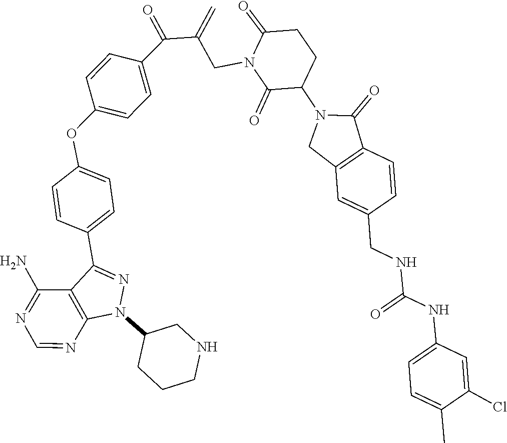 [SMILES: C=C(CN1C(=O)CCC(N2Cc3cc(CNC(=O)Nc4ccc(C)c(Cl)c4)ccc3C2=O)C1=O)C(=O)c1ccc(Oc2ccc(-c3nn([C@@H]4CCCNC4)c4ncnc(N)c34)cc2)cc1] |
| III-4 | 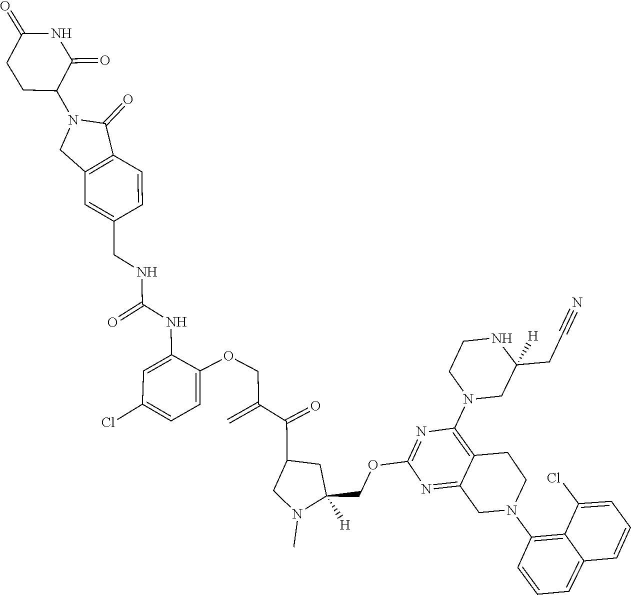 [SMILES: C=C(COc1ccc(Cl)cc1NC(=O)NCc1ccc2c(c1)CN(C1CCC(=O)NC1=O)C2=O)C(=O)C1C[C@@H](COc2nc3c(c(N4CCN[C@@H](CC#N)C4)n2)CCN(c2cccc4cccc(Cl)c24)C3)N(C)C1] |
| III-5 | 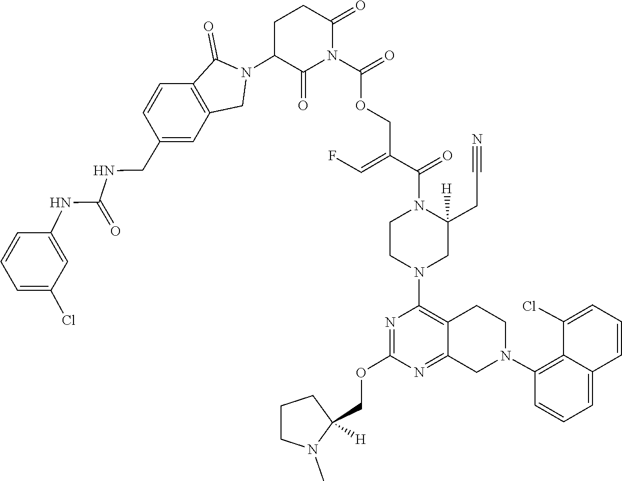 [SMILES: CN1CCC[C@H]1COc1nc2c(c(N3CCN(C(=O)/C(=C/F)COC(=O)N4C(=O)CCC(N5Cc6cc(CNC(=O)Nc7cccc(Cl)c7)ccc6C5=O)C4=O)[C@@H](CC#N)C3)n1)CCN(c1cccc3cccc(Cl)c13)C2] |
| III-6 | 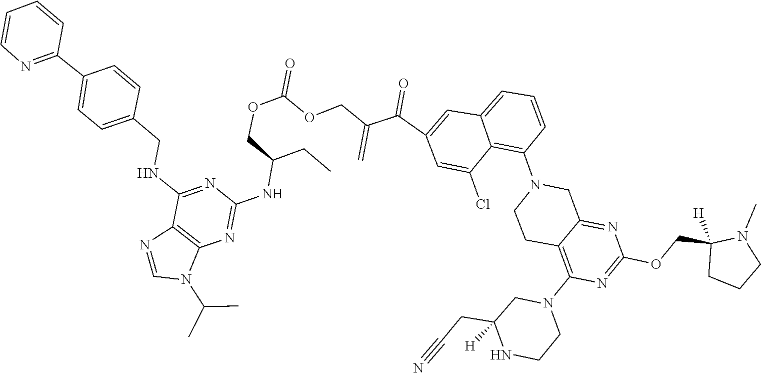 [SMILES: C=C(COC(=O)OC[C@@H](CC)Nc1nc(NCc2ccc(-c3ccccn3)cc2)c2ncn(C(C)C)c2n1)C(=O)c1cc(Cl)c2c(N3CCc4c(nc(OC[C@@H]5CCCN5C)nc4N4CCN[C@@H](CC#N)C4)C3)cccc2c1] |
| III-7 | 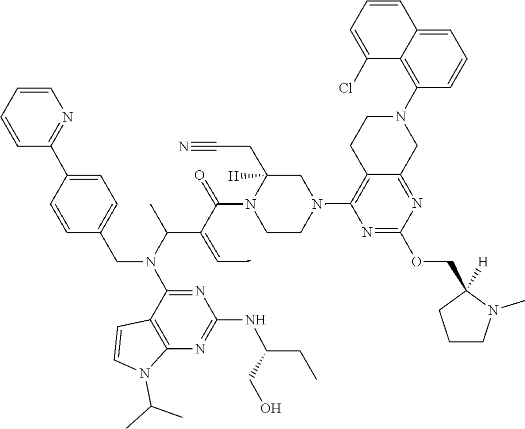 [SMILES: C/C=C(\C(=O)N1CCN(c2nc(OC[C@@H]3CCCN3C)nc3c2CCN(c2cccc4cccc(Cl)c24)C3)C[C@@H]1CC#N)C(C)N(Cc1ccc(-c2ccccn2)cc1)c1nc(N[C@H](CC)CO)nc2c1ccn2C(C)C] |
| III-8 | 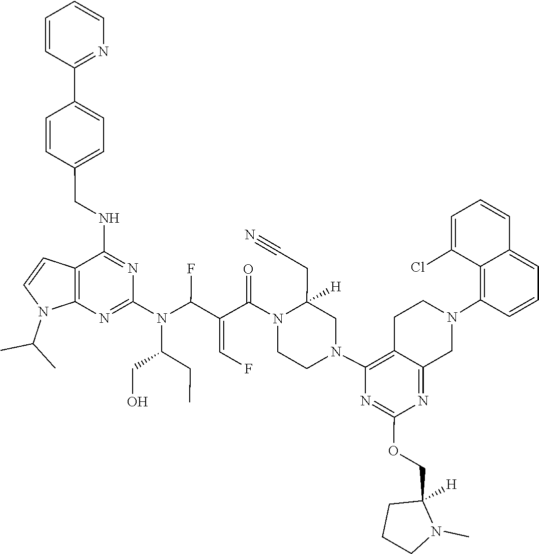 [SMILES: CC[C@H](CO)N(c1nc(NCc2ccc(-c3ccccn3)cc2)c2ccn(C(C)C)c2n1)C(F)/C(=C/F)C(=O)N1CCN(c2nc(OC[C@@H]3CCCN3C)nc3c2CCN(c2cccc4cccc(Cl)c24)C3)C[C@@H]1CC#N] |
| III-9 | 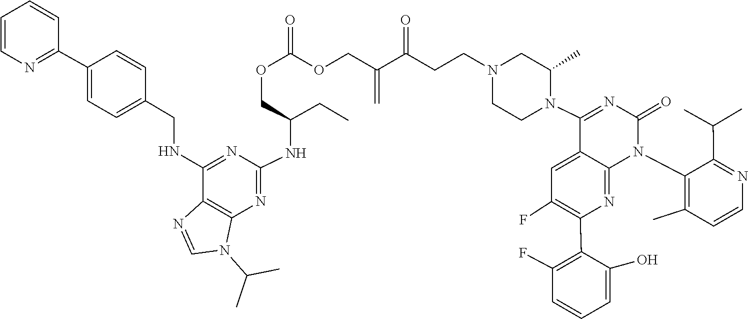 [SMILES: C=C(COC(=O)OC[C@@H](CC)Nc1nc(NCc2ccc(-c3ccccn3)cc2)c2ncn(C(C)C)c2n1)C(=O)CCN1CCN(c2nc(=O)n(-c3c(C)ccnc3C(C)C)c3nc(-c4c(O)cccc4F)c(F)cc23)[C@@H](C)C1] |
| III-10 | 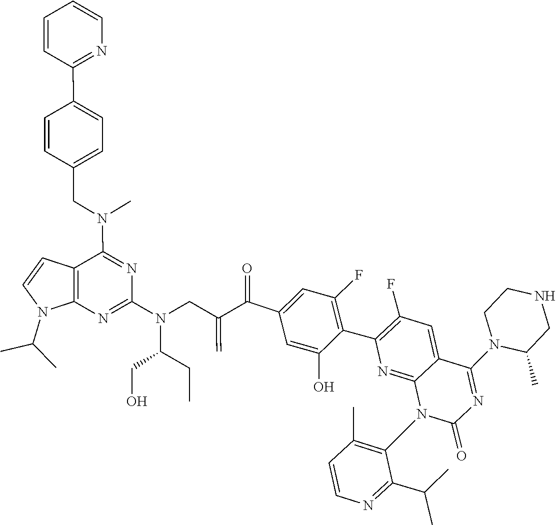 [SMILES: C=C(CN(c1nc(N(C)Cc2ccc(-c3ccccn3)cc2)c2ccn(C(C)C)c2n1)[C@H](CC)CO)C(=O)c1cc(O)c(-c2nc3c(cc2F)c(N2CCNC[C@@H]2C)nc(=O)n3-c2c(C)ccnc2C(C)C)c(F)c1] |
| III-11 | 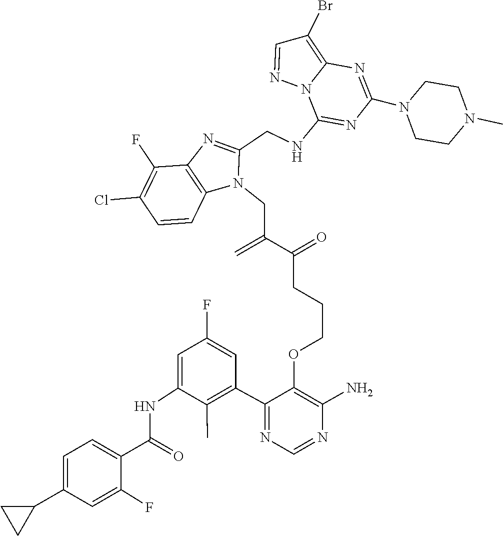 [SMILES: C=C(Cn1c(CNc2nc(N3CCN(C)CC3)nc3c(Br)cnn23)nc2c(F)c(Cl)ccc21)C(=O)CCCOc1c(N)ncnc1-c1cc(F)cc(NC(=O)c2ccc(C3CC3)cc2F)c1C] |
| III-12 | 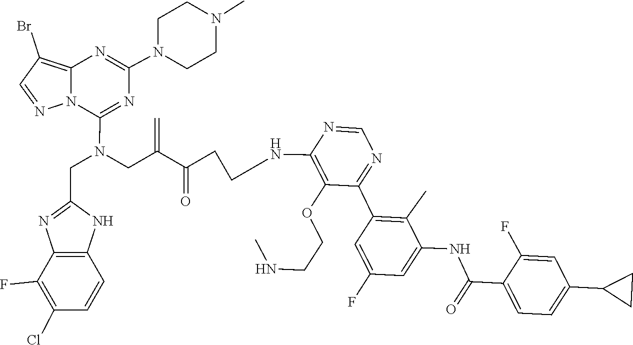 [SMILES: C=C(CN(Cc1nc2c(F)c(Cl)ccc2[nH]1)c1nc(N2CCN(C)CC2)nc2c(Br)cnn12)C(=O)CCNc1ncnc(-c2cc(F)cc(NC(=O)c3ccc(C4CC4)cc3F)c2C)c1OCCNC] |
| III-13 | 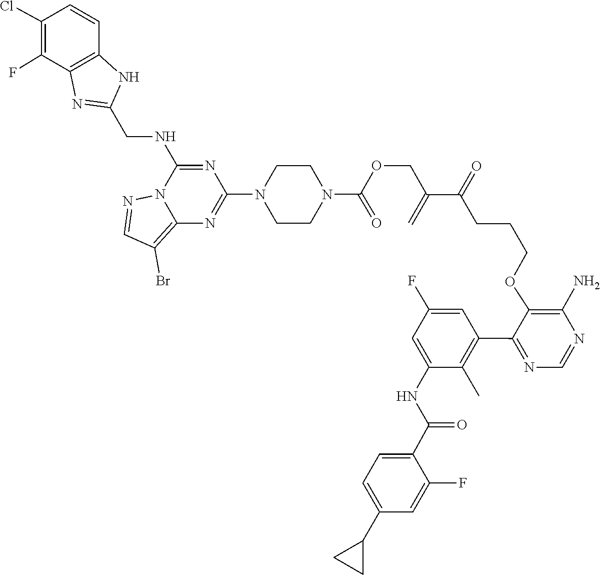 [SMILES: C=C(COC(=O)N1CCN(c2nc(NCc3nc4c(F)c(Cl)ccc4[nH]3)n3ncc(Br)c3n2)CC1)C(=O)CCCOc1c(N)ncnc1-c1cc(F)cc(NC(=O)c2ccc(C3CC3)cc2F)c1C] |
| III-14 | 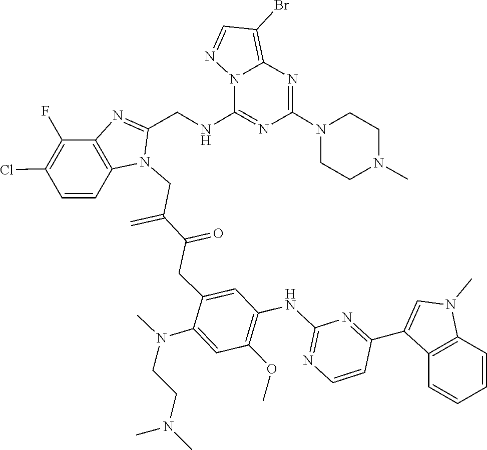 [SMILES: C=C(Cn1c(CNc2nc(N3CCN(C)CC3)nc3c(Br)cnn23)nc2c(F)c(Cl)ccc21)C(=O)Cc1cc(Nc2nccc(-c3cn(C)c4ccccc34)n2)c(OC)cc1N(C)CCN(C)C] |
| III-15 | 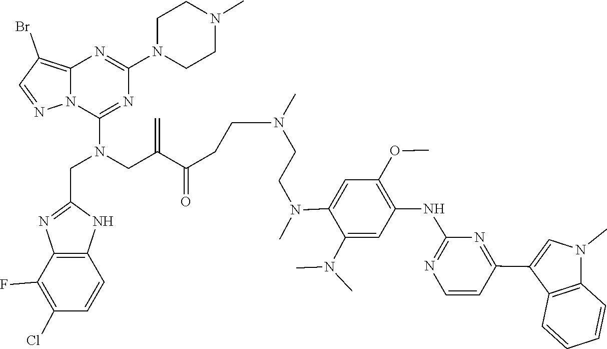 [SMILES: C=C(CN(Cc1nc2c(F)c(Cl)ccc2[nH]1)c1nc(N2CCN(C)CC2)nc2c(Br)cnn12)C(=O)CCN(C)CCN(C)c1cc(OC)c(Nc2nccc(-c3cn(C)c4ccccc34)n2)cc1N(C)C] |
| III-16 | 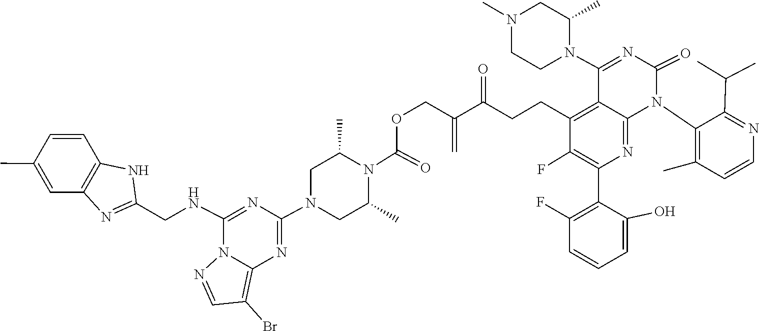 [SMILES: C=C(COC(=O)N1[C@H](C)CN(c2nc(NCc3nc4cc(C)ccc4[nH]3)n3ncc(Br)c3n2)C[C@@H]1C)C(=O)CCc1c(F)c(-c2c(O)cccc2F)nc2c1c(N1CCN(C)C[C@@H]1C)nc(=O)n2-c1c(C)ccnc1C(C)C] |
| III-17 | 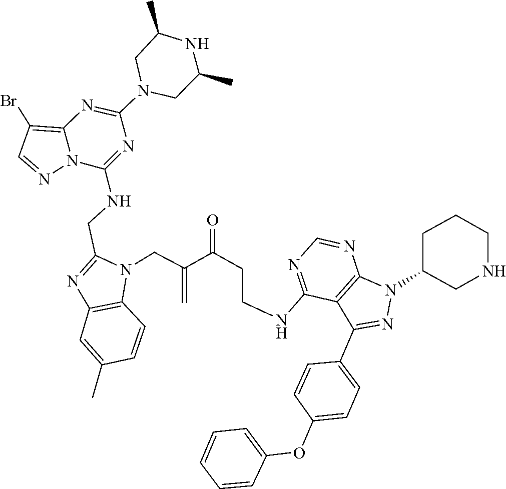 [SMILES: C=C(Cn1c(CNc2nc(N3C[C@@H](C)N[C@@H](C)C3)nc3c(Br)cnn23)nc2cc(C)ccc21)C(=O)CCNc1ncnc2c1c(-c1ccc(Oc3ccccc3)cc1)nn2[C@@H]1CCCNC1] |
| III-18 | 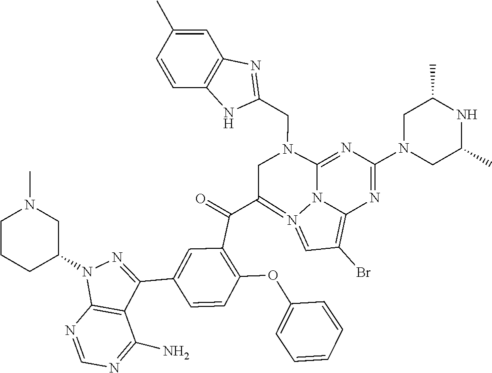 |
| III-19 | 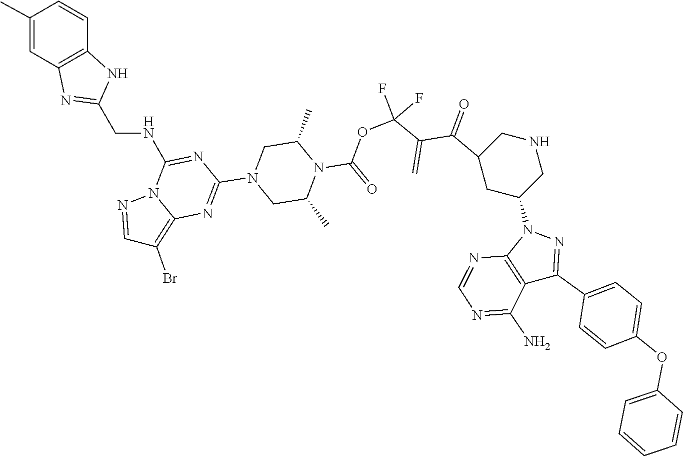 [SMILES: C=C(C(=O)C1CNC[C@H](n2nc(-c3ccc(Oc4ccccc4)cc3)c3c(N)ncnc32)C1)C(F)(F)OC(=O)N1[C@H](C)CN(c2nc(NCc3nc4cc(C)ccc4[nH]3)n3ncc(Br)c3n2)C[C@@H]1C] |
| III-20 | 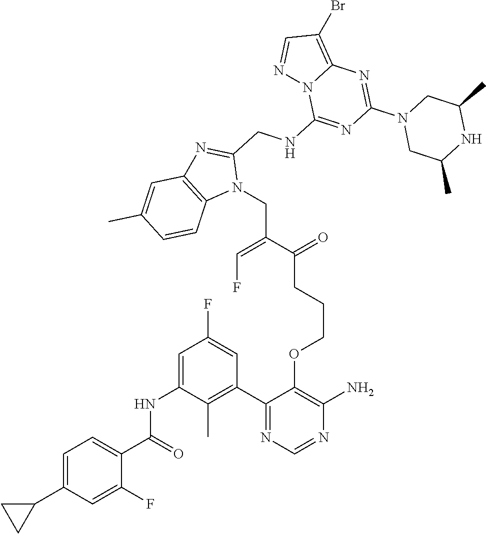 [SMILES: Cc1ccc2c(c1)nc(CNc1nc(N3C[C@@H](C)N[C@@H](C)C3)nc3c(Br)cnn13)n2C/C(=C/F)C(=O)CCCOc1c(N)ncnc1-c1cc(F)cc(NC(=O)c2ccc(C3CC3)cc2F)c1C] |

In certain embodiments, the compound is a compound in Table 4, or a pharmaceutically acceptable salt thereof.

TABLE 4
Additional Compounds

| No. | Chemical Structure |
|---|---|
| IV-1 | 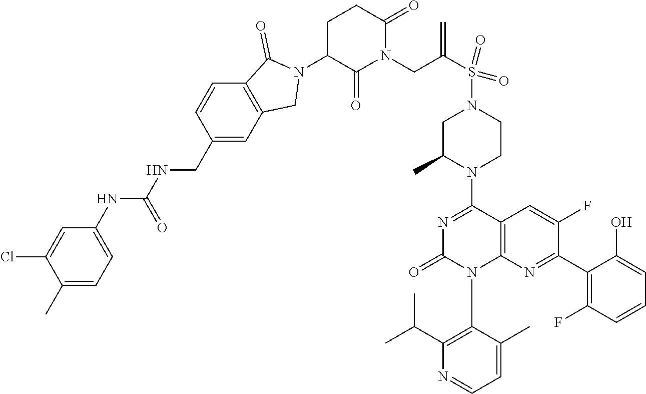 [SMILES: C=C(CN1C(=O)CCC(N2Cc3cc(CNC(=O)Nc4ccc(C)c(Cl)c4)ccc3C2=O)C1=O)S(=O)(=O)N1CCN(c2nc(=O)n(-c3c(C)ccnc3C(C)C)c3nc(-c4c(O)cccc4F)c(F)cc23)[C@@H](C)C1] |
| IV-2 | 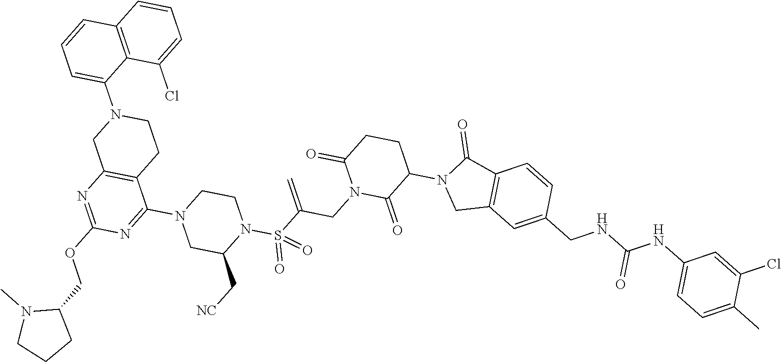 [SMILES: C=C(CN1C(=O)CCC(N2Cc3cc(CNC(=O)Nc4ccc(C)c(Cl)c4)ccc3C2=O)C1=O)S(=O)(=O)N1CCN(c2nc(OC[C@@H]3CCCN3C)nc3c2CCN(c2cccc4cccc(Cl)c24)C3)C[C@@H]1CC#N] |
| IV-3 | 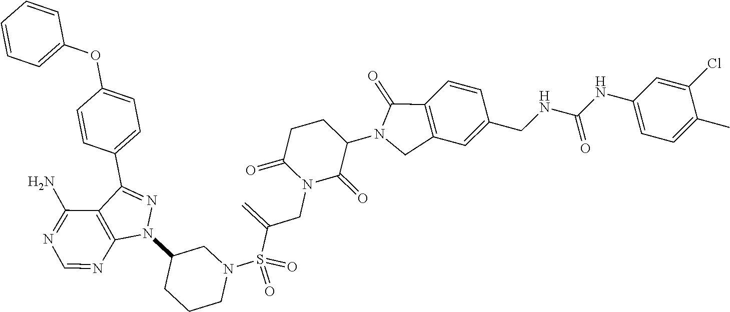 |
| IV-4 | 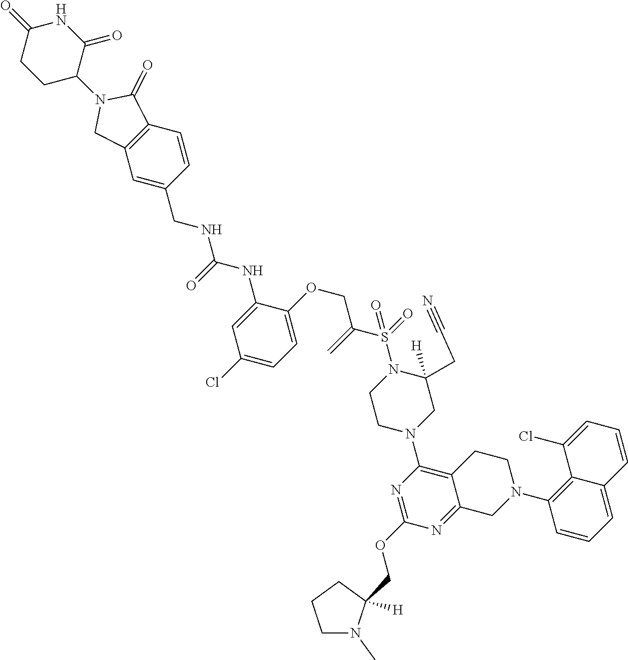 [SMILES: C=C(COc1ccc(Cl)cc1NC(=O)NCc1ccc2c(c1)CN(C1CCC(=O)NC1=O)C2=O)S(=O)(=O)N1CCN(c2nc(OC[C@@H]3CCCN3C)nc3c2CCN(c2cccc4cccc(Cl)c24)C3)C[C@@H]1CC#N] |
| IV-5 | 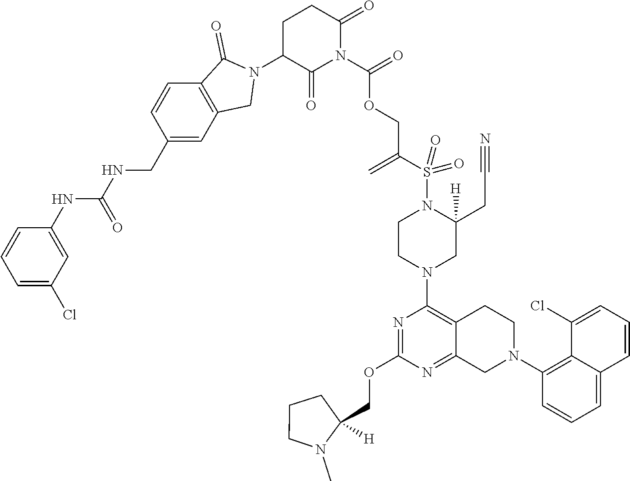 [SMILES: C=C(COC(=O)N1C(=O)CCC(N2Cc3cc(CNC(=O)Nc4cccc(Cl)c4)ccc3C2=O)C1=O)S(=O)(=O)N1CCN(c2nc(OC[C@@H]3CCCN3C)nc3c2CCN(c2cccc4cccc(Cl)c24)C3)C[C@@H]1CC#N] |
| IV-6 | 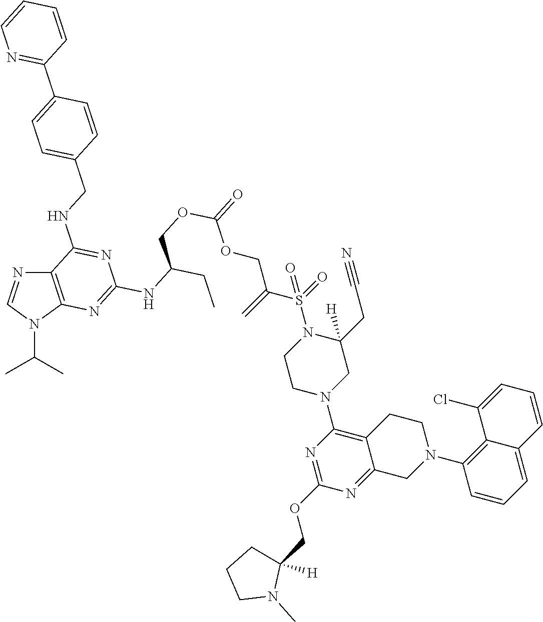 [SMILES: C=C(COC(=O)OC[C@@H](CC)Nc1nc(NCc2ccc(-c3ccccn3)cc2)c2ncn(C(C)C)c2n1)S(=O)(=O)N1CCN(c2nc(OC[C@@H]3CCCN3C)nc3c2CCN(c2cccc4cccc(Cl)c24)C3)C[C@@H]1CC#N] |
| IV-7 | 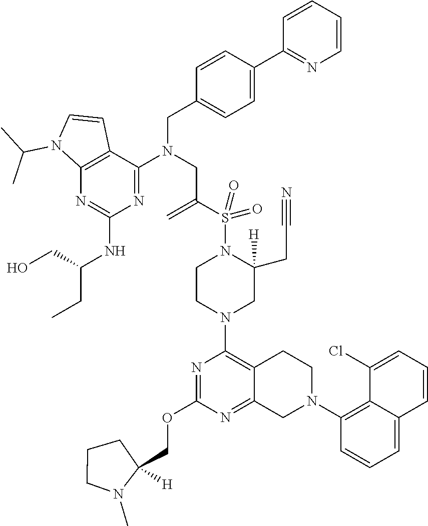 [SMILES: C=C(CN(Cc1ccc(-c2ccccn2)cc1)c1nc(N[C@H](CC)CO)nc2c1ccn2C(C)C)S(=O)(=O)N1CCN(c2nc(OC[C@@H]3CCCN3C)nc3c2CCN(c2cccc4cccc(Cl)c24)C3)C[C@@H]1CC#N] |
| IV-8 | 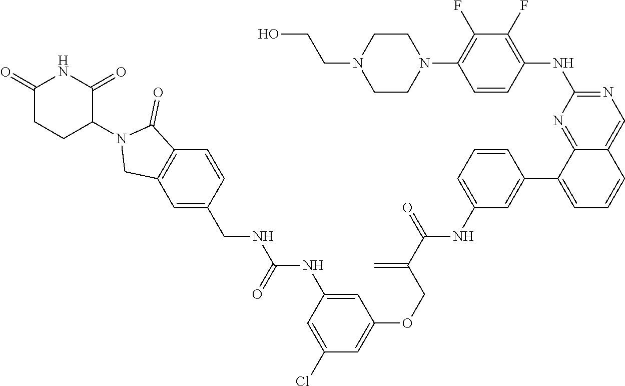 [SMILES: C=C(COc1cc(Cl)cc(NC(=O)NCc2ccc3c(c2)CN(C2CCC(=O)NC2=O)C3=O)c1)C(=O)Nc1cccc(-c2cccc3cnc(Nc4ccc(N5CCN(CCO)CC5)c(F)c4F)nc23)c1] |
| IV-9 | 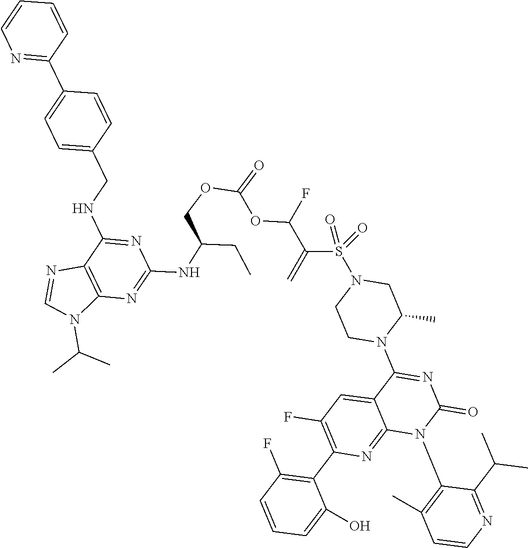 [SMILES: C=C(C(F)OC(=O)OC[C@@H](CC)Nc1nc(NCc2ccc(-c3ccccn3)cc2)c2ncn(C(C)C)c2n1)S(=O)(=O)N1CCN(c2nc(=O)n(-c3c(C)ccnc3C(C)C)c3nc(-c4c(O)cccc4F)c(F)cc23)[C@@H](C)C1] |
| IV-10 | 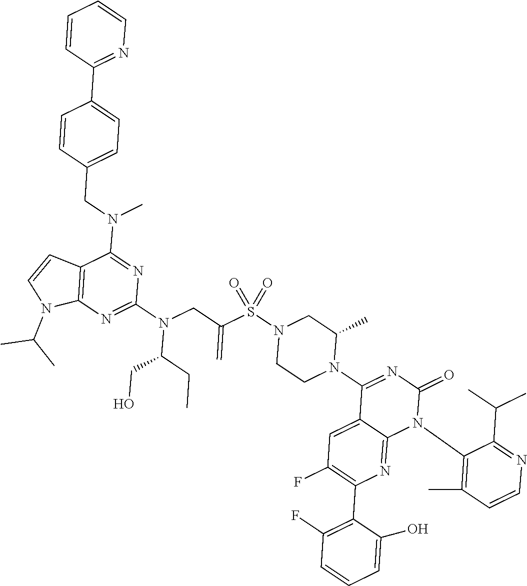 [SMILES: C=C(CN(c1nc(N(C)Cc2ccc(-c3ccccn3)cc2)c2ccn(C(C)C)c2n1)[C@H](CC)CO)S(=O)(=O)N1CCN(c2nc(=O)n(-c3c(C)ccnc3C(C)C)c3nc(-c4c(O)cccc4F)c(F)cc23)[C@@H](C)C1] |
| IV-11 | 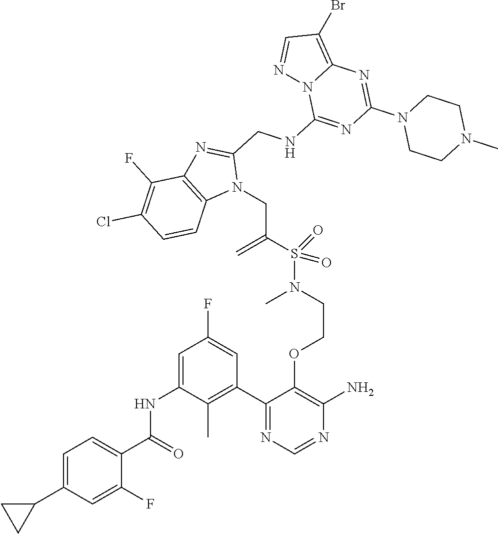 [SMILES: C=C(Cn1c(CNc2nc(N3CCN(C)CC3)nc3c(Br)cnn23)nc2c(F)c(Cl)ccc21)S(=O)(=O)N(C)CCOc1c(N)ncnc1-c1cc(F)cc(NC(=O)c2ccc(C3CC3)cc2F)c1C] |
| IV-12 | 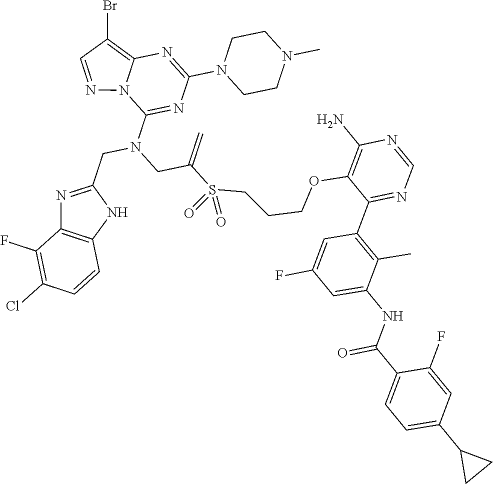 [SMILES: C=C(CN(Cc1nc2c(F)c(Cl)ccc2[nH]1)c1nc(N2CCN(C)CC2)nc2c(Br)cnn12)S(=O)(=O)CCCOc1c(N)ncnc1-c1cc(F)cc(NC(=O)c2ccc(C3CC3)cc2F)c1C] |
| IV-13 | 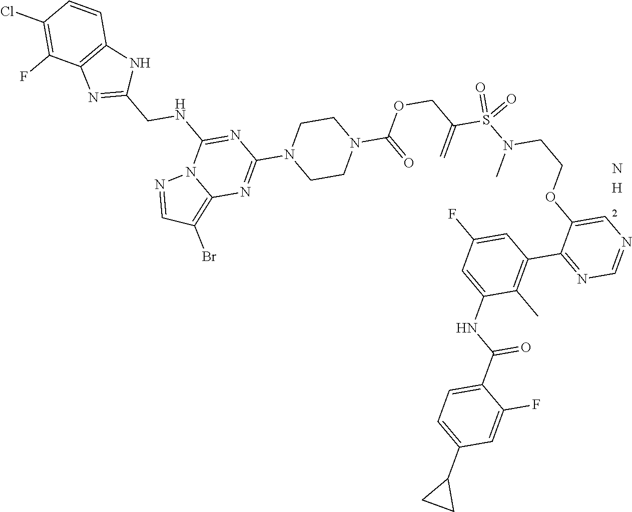 [SMILES: C=C(COC(=O)N1CCN(c2nc(NCc3nc4c(F)c(Cl)ccc4[nH]3)n3ncc(Br)c3n2)CC1)S(=O)(=O)N(C)CCOc1cncnc1-c1cc(F)cc(NC(=O)c2ccc(C3CC3)cc2F)c1C] |
| IV-14 | 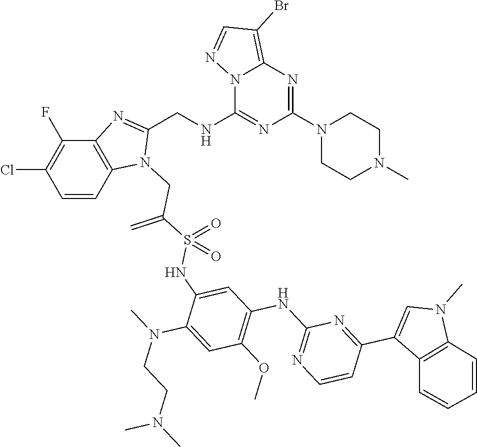 [SMILES: C=C(Cn1c(CNc2nc(N3CCN(C)CC3)nc3c(Br)cnn23)nc2c(F)c(Cl)ccc21)S(=O)(=O)Nc1cc(Nc2nccc(-c3cn(C)c4ccccc34)n2)c(OC)cc1N(C)CCN(C)C] |
| IV-15 | 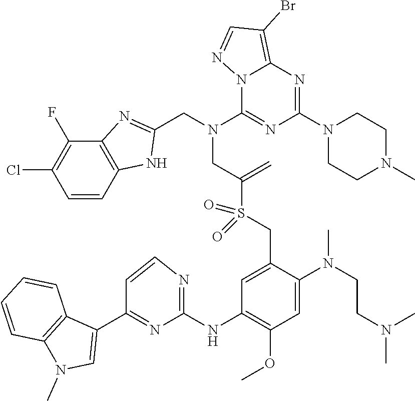 [SMILES: C=C(CN(Cc1nc2c(F)c(Cl)ccc2[nH]1)c1nc(N2CCN(C)CC2)nc2c(Br)cnn12)S(=O)(=O)Cc1cc(Nc2nccc(-c3cn(C)c4ccccc34)n2)c(OC)cc1N(C)CCN(C)C] |
| IV-16 | 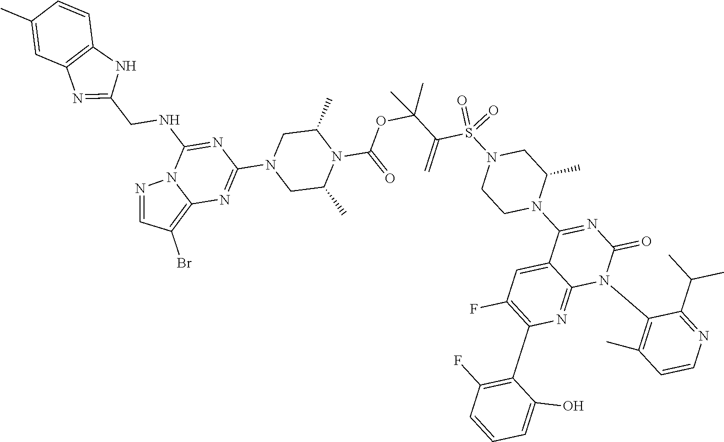 [SMILES: C=C(C(C)(C)OC(=O)N1[C@H](C)CN(c2nc(NCc3nc4cc(C)ccc4[nH]3)n3ncc(Br)c3n2)C[C@@H]1C)S(=O)(=O)N1CCN(c2nc(=O)n(-c3c(C)ccnc3C(C)C)c3nc(-c4c(O)cccc4F)c(F)cc23)[C@@H](C)C1] |
| IV-17 | 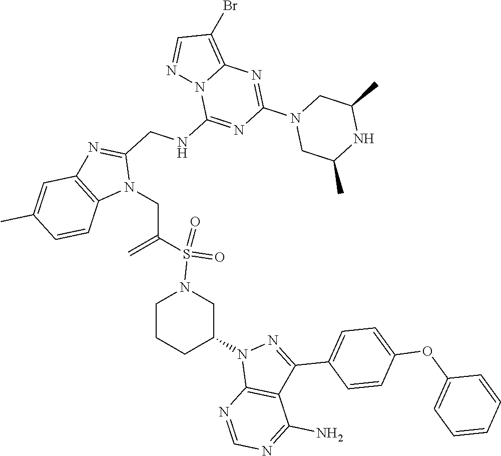 [SMILES: C=C(Cn1c(CNc2nc(N3C[C@@H](C)N[C@@H](C)C3)nc3c(Br)cnn23)nc2cc(C)ccc21)S(=O)(=O)N1CCC[C@@H](n2nc(-c3ccc(Oc4ccccc4)cc3)c3c(N)ncnc32)C1] |
| IV-18 | 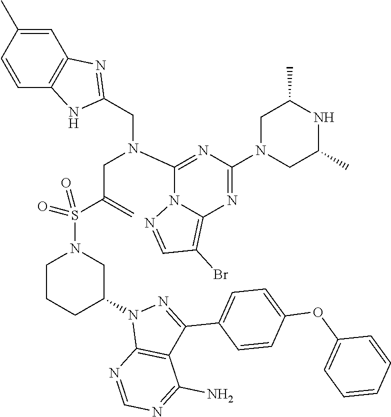 [SMILES: C=C(CN(Cc1nc2cc(C)ccc2[nH]1)c1nc(N2C[C@@H](C)N[C@@H](C)C2)nc2c(Br)cnn12)S(=O)(=O)N1CCC[C@@H](n2nc(-c3ccc(Oc4ccccc4)cc3)c3c(N)ncnc32)C1] |
| IV-19 | 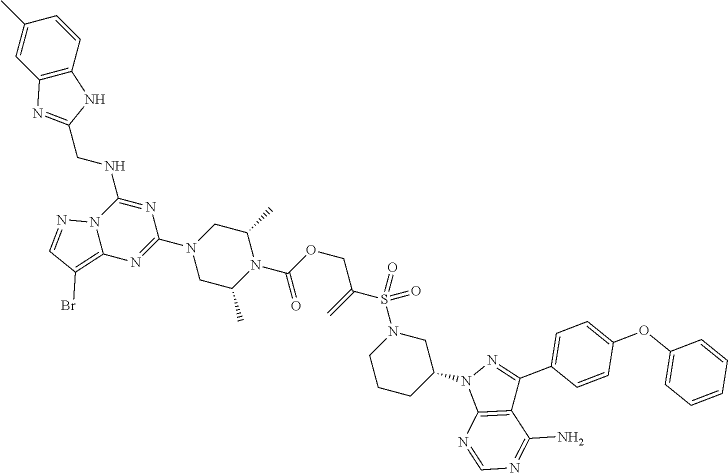 [SMILES: C=C(COC(=O)N1[C@H](C)CN(c2nc(NCc3nc4cc(C)ccc4[nH]3)n3ncc(Br)c3n2)C[C@@H]1C)S(=O)(=O)N1CCC[C@@H](n2nc(-c3ccc(Oc4ccccc4)cc3)c3c(N)ncnc32)C1] |
| IV-20 | 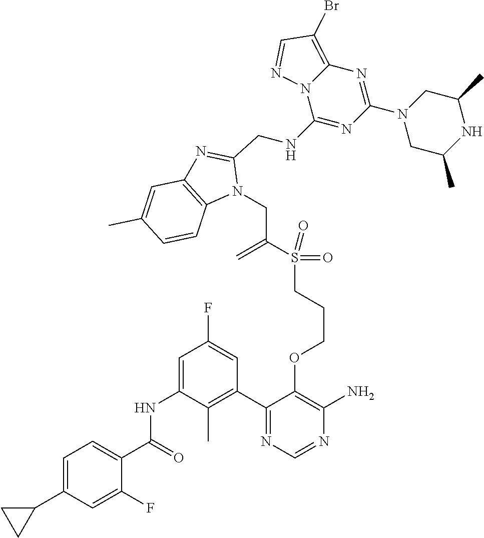 [SMILES: C=C(Cn1c(CNc2nc(N3C[C@@H](C)N[C@@H](C)C3)nc3c(Br)cnn23)nc2cc(C)ccc21)S(=O)(=O)CCCOc1c(N)ncnc1-c1cc(F)cc(NC(=O)c2ccc(C3CC3)cc2F)c1C] |

### Synthetic Methods

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing heterobifunctional compounds D′. Reacting compound A′ (a precursor of EPL, for example, a discrete compound that is an effector protein ligand) with 3-bromo-2-(bromomethyl)propanoic acid affords intermediate B′. Reacting intermediate B′ with compound C′ (a precursor of TPL) under amide-coupling conditions affords heterobifunctional compound D′. It is understood by one skilled in the art of organic synthesis that protecting group strategies may be employed as necessary.

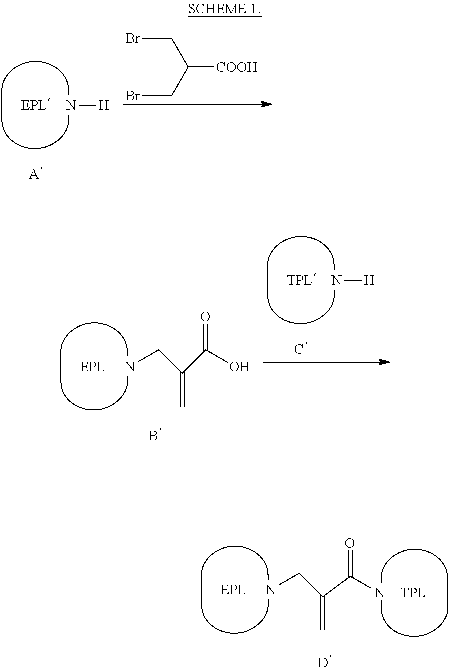

### II. Therapeutic Applications

The heterobifunctional compounds described herein, such as a compound of Formula I or other compounds in Section I, provide therapeutic benefits to patients suffering from cancer. Accordingly, one aspect of the invention provides a method of treating cancer. The method comprises administering to a patient in need thereof a therapeutically effective amount of a compound described herein, such as a compound of Formula I or other compounds in Section I, to treat the cancer. In certain embodiments, the particular compound of Formula I is a compound defined by one of the embodiments described above.

### Cancer

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, espophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

### Causing Death of Cancer Cell

Another aspect of the invention provides a method of causing death of a cancer cell. The method comprises contacting a cancer cell with an effective amount of a compound described herein, such as a compound of Formula I or other compounds in Section I, to cause death of the cancer cell. In certain embodiments, the particular compound of Formula I is a compound defined by one of the embodiments described above.

In certain embodiments, the cancer cell is selected from ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer cell is one or more of the cancers recited in the section above entitled “Cancer.”

### Protein Degradation

Another aspect of the invention provides a method of degrading an effector protein in a cell, wherein the method comprises administering to the cell an effective amount of a compound described herein, such as a compound of Formula I, resulting in degradation of the effector protein in the cell, wherein the effector protein is GSPT1, Cyclin K, RBM23, RBM39, IKZF1, IKZF3, PLK1, CDK4, or CK1alpha. In certain embodiments, the effector protein is GSPT1. In certain embodiments, the effector protein is Cyclin K. In certain embodiments, the effector protein is RBM23. In certain embodiments, the effector protein is RBM39. In certain embodiments, the effector protein is IKZF1. In certain embodiments, the effector protein is IKZF3. In certain embodiments, the effector protein is PLK1. In certain embodiments, the effector protein is CDK4. In certain embodiments, the effector protein is CK1alpha.

In certain embodiments, the cell is a cell that expresses a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4. In certain embodiments, the cell is a cancer cell that expresses a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4. In certain embodiments, the target protein is KRAS. In certain embodiments, the target protein is HER2. In certain embodiments, the target protein is BTK. In certain embodiments, the target protein is EGFR. In certain embodiments, the target protein is androgen receptor protein. In certain embodiments, the target protein is estrogen receptor protein. In certain embodiments, the target protein is ALK. In certain embodiments, the target protein is IDH1. In certain embodiments, the target protein is FLT3. In certain embodiments, the target protein is FGFR1. In certain embodiments, the target protein is FGFR4. In certain embodiments, the target protein is FGFR2. In certain embodiments, the target protein is FGFR3. In certain embodiments, the target protein is ERK1. In certain embodiments, the target protein is ERK2. In certain embodiments, the target protein is FGR. In certain embodiments, the target protein is HER3. In certain embodiments, the cell is a cancer cell, wherein the cancer is one of those described above in the section entitled “Cancer.”

Another aspect of the invention provides a method of degrading a GSPT1 protein in a cell, wherein the method comprises administering to the cell an effective amount of a compound described herein, such as a compound of Formula I, resulting in degradation of the GSPT1 protein in the cell. In certain embodiments, the cell is a cell that expresses a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4. In certain embodiments, the cell is a cancer cell that expresses a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4. In certain embodiments, the target protein is KRAS. In certain embodiments, the target protein is HER2. In certain embodiments, the target protein is BTK. In certain embodiments, the target protein is EGFR. In certain embodiments, the target protein is androgen receptor protein. In certain embodiments, the target protein is estrogen receptor protein. In certain embodiments, the target protein is ALK. In certain embodiments, the target protein is IDH1. In certain embodiments, the target protein is FLT3. In certain embodiments, the target protein is FGFR1. In certain embodiments, the target protein is FGFR4. In certain embodiments, the target protein is FGFR2. In certain embodiments, the target protein is FGFR3. In certain embodiments, the target protein is ERK1. In certain embodiments, the target protein is ERK2. In certain embodiments, the target protein is FGR. In certain embodiments, the target protein is HER3. In certain embodiments, the cell is a cancer cell, wherein the cancer is one of those described above in the section entitled “Cancer.”

### Combination Therapies

The compounds useful within the methods of the invention may be used in combination with one or more additional therapeutic agents useful for treating any disease contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein.

Accordingly, in certain embodiments, the method further comprises administering to the subject an additional therapeutic agent that treats the disease contemplated herein.

In certain embodiments, administering the compound of the invention to the subject allows for administering a lower dose of the additional therapeutic agent as compared to the dose of the additional therapeutic agent alone that is required to achieve similar results in treating the disease contemplated herein. For example, in certain embodiments, the compound of the invention enhances the therapeutic activity of the additional therapeutic compound, thereby allowing for a lower dose of the additional therapeutic compound to provide the same effect.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound of the invention and the therapeutic agent are co-administered to the subject. In other embodiments, the compound of the invention and the therapeutic agent are coformulated and co-administered to the subject.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In certain embodiments, the second therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In certain embodiments, the second therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGFO trap compound (NCT02699515); and (NCT02517398). M7824 is comprised of a fully human IgGI antibody against PD-L1 fused to the extracellular domain of human TGF-beta receptor II, which functions as a TGFO “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Another aspect of the invention provides for the use of a compound described herein (such as a compound of Formula I or other compounds in Section I) in the manufacture of a medicament. In certain embodiments, the medicament is for treating a disease described herein, such as cancer.

Another aspect of the invention provides for the use of a compound described herein (such as a compound of Formula I or other compounds in Section I) for treating a medical disease, such a disease described herein (e.g., cancer).

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a heterobifunctional substituted phenylpyrimidinone or related compound described herein in a therapeutically effective amount for the treatment of a medical disorder described herein.

### IV. Medical Kits

Another aspect of this invention is a kit comprising (i) a compound described herein, such as a compound of Formula I, and (ii) instructions for use, such as treating cancer.

### EXAMPLES

The invention now being generally described, will be more readily understood by reference to the following examples, which are included merely for purposes of illustration of certain aspects and embodiments of the present invention, and are not intended to limit the invention.

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropylethylamine (DIPEA) was obtained anhydrous by distillation over potassium hydroxide. Tetrahydrofuran (THF), Dichloromethane (CH₂Cl₂), and dimethylformamide (DMF) was dried by a PureSolv™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO4.

¹H and ¹³C NMR spectra were recorded on an Agilent DD₂ 500 (500 MHz ¹H; 125 MHz ¹³C) or Agilent DD₂ 600 (600 MHz ¹H; 150 MHz ¹³C) or Agilent DD₂ 400 (400 MHz ¹H; 100 MHz ¹³C) spectrometer at room temperature. Chemical shifts were reported in ppm relative to the residual CDCl₃ (δ 7.26 ppm ¹H; δ 77.0 ppm ¹³C), CD₃OD (δ 3.31 ppm ¹H; δ 49.00 ppm ¹³C), or d₆-DMSO (δ 2.50 ppm ¹H; δ 39.52 ppm ¹³C). NMR chemical shifts were expressed in ppm relative to internal solvent peaks, and coupling constants were measured in Hz. (bs=broad signal). In most cases, only peaks of the major rotamer are reported.

Mass spectra were obtained using Agilent 1100 series LC/MSD spectrometers. Analytical HPLC analyses were carried out on 250×4.6 mm C-18 column using gradient conditions (10-100% B, flow rate=1.0 mL/min, 20 min), or as described in the LC-MS Method tables.

Unless indicated otherwise, preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA). Final products were typically purified via reversed-phase HPLC, PTLC, or flash column chromatography.

LC-MS Method A

| Instrument | | Agilent 1290 Infinity II UPLC & Agilent 6120B MSD | | |
|---|---|---|---|---|
| Software | | Rev. C.01.10 [239] | | |
| HPLC | Column | Waters Acquity UPLC CSH C18 1.7 μm, 2.1 × 30 mm | | |
| | Mobile Phase | A: 0.1% formic acid in H₂O | | |
| | | B: acetonitrile | | |
| | Gradient | Time (min) | B (%) | Flow (mL/min) |
| | | 0.00 | 5 | 0.9 mL/min |
| | | 3.0 | 95 | 0.9 mL/min |
| | | 3.40 | 95 | 0.9 mL/min |
| | Post time(min) | n.a. | | |
| | Column Temp | 40° C. | | |
| | Detector | Photodiode-Array Detection (PAD) | | |
| MS | Ionization source | ESI | | |
| | Drying Gas | N₂ | | |
| | Drying Gas Flow | 12.0 L/min | | |
| | Nebulizer Pressure | 35 (psig) | | |
| | Drying Gas | 350° C. | | |
| | Temperature | | | |

| | |
|---|---|
| Capillary Voltage | 3000 (V) |
| MS Polarity | Positive-Negative |
| MS Mode | Scan |
| Mass Range | 100-1000 |

LC-MS Method B

| Instrument | | Waters Acquity H-Class UPLC & QDa MSD | | |
|---|---|---|---|---|
| Software | | Masslynx 4.2 SCN976 | | |
| HPLC | Column | Waters Acquity UPLC CSH C18 1.7 μm, 2.1 × 30 mm | | |
| | Mobile Phase | A: 0.1% formic acid in H₂O | | |
| | | B: acetonitrile | | |
| | Gradient | Time (min) | B (%) | Flow (mL/min) |
| | | 0.00 | 5 | 0.9 mL/min |
| | | 2.0 | 100 | 0.9 mL/min |
| | | 2.7 | 100 | 0.9 mL/min |
| | Post time(min) | n.a. | | |
| | Column Temp | 40° C. | | |
| | Detector | Photodiode-Array Detection (PAD) | | |
| MS | Ionization source | ESI | | |
| | Drying Gas | N₂ | | |
| | Drying Gas Flow | n.a. | | |
| | Nebulizer Pressure | n.a. | | |
| | Drying Gas | n.a. | | |
| | Temperature | | | |

| | |
|---|---|
| Capillary Voltage | 0.8 (kV) |
| MS Polarity | Positive-Negative |
| MS Mode | Scan |
| Mass Range | 100-1200 |

LC-MS Method C

| Instrument | | Agilent 1260 Infinity LC & Agilent 6120A MSD | | |
|---|---|---|---|---|
| Software | | Rev. C.01.07SR1 [113] | | |
| HPLC | Column | Phenomenex Kinetex 2.6 μm Evo C18, 50 × 3.0 mm | | |
| | Mobile Phase | A: 0.1% formic acid in H₂O | | |
| | | B: acetonitrile | | |
| | Gradient | Time (min) | B (%) | Flow (mL/min) |
| | | 0.00 | 10 | 1.0 mL/min |
| | | 4.50 | 95 | 1.0 mL/min |

| | | |
|---|---|---|
| | Post time(min) | n.a. |
| | Column Temp | 40° C. |
| | Detector | Photodiode-Array Detection (PAD) |
| MS | Ionization source | ESI |
| | Drying Gas | N₂ |
| | Drying Gas Flow | 12.0 L/min |
| | Nebulizer Pressure | 35 (psig) |
| | Drying Gas | 350° C. |
| | Temperature | |
| | Capillary Voltage | 3000 (V) |
| | MS Polarity | Positive |
| | MS Mode | Scan |
| | Mass Range | 100-800 or 100-1200 |

LC-MS Method D

| Instrument | | Agilent 1260 Infinity II LC & Agilent MSD XT | | |
|---|---|---|---|---|
| Software | | Agilent Chemstation Rev. C. 01.09 [144] | | |
| HPLC | Column | Waters Acquity CSH C18, 1.7 μm, 2.1 × 5 0 mm | | |
| | Mobile Phase | A: 0.1% Formic acid in 95/5 Water/Acetonitrile (v/v) | | |
| | | B: 0.08% Formic acid in 95/5 Acetonitrile/Water (v/v) | | |
| | Gradient | Time(min) | B(%) | Flow(mL/min) |
| | | 0.00 | 0 | 0.6 |
| | | 2.5 | 100 | 0.6 |
| | | 3.5 | 100 | 0.6 |
| | | 3.51 | 0 | 0.6 |
| | | 4.00 | 0 | 0.6 |

| | | |
|---|---|---|
| | Post time(min) | 0 |
| | Column Temp | 35° C. |
| | Detector | DAD |
| MS | Ionization source | ESI |
| | Drying Gas | N₂ |
| | Drying Gas Flow | 12 (L/min) |
| | Nebulizer Pressure | 1810 (Torr) |
| | Drying Gas | 350° C. |
| | Temperature | |
| | Capillary Voltage | 3000(V) Positive |
| | MS Polarity | Positive |
| | MS Mode | Scan |
| | Mass Range | 180-2000 |

### LC-MS Method AB01

AB01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |
| | Post time(min) | 0 | | |
| | Column Temp | 50° C. | | |
| | Detector | DAD | | |
| MS | Ionization source | ESI | | |
| | Drying Gas | N₂ | | |
| | Drying Gas Flow | 10(L/min) | | |
| | Nebulizer Pressure | 40(psi) | | |
| | Drying Gas | 350° C. | | |
| | Temperature | | | |

| | |
|---|---|
| Capillary Voltage | 2500(V) |
| MS Polarity | Positive |
| MS Mode | Scan |
| Mass Range | 100-1500 |

### LC-MS Method AB05

AB05

| Instrument | | SHIMADZU LCMS-2020; | | |
|---|---|---|---|---|
| Software | | LabSolution Version 5.93 | | |
| HPLC | Column | Kinetex EVO C18 2.1 × 30 mm, 5 μm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.0 | 5 | 1.5 |
| | | 0.80 | 95 | 1.5 |
| | | 1.20 | 95 | 1.5 |
| | | 1.21 | 5 | 1.5 |
| | | 1.55 | 5 | 1.5 |

| | | |
|---|---|---|
| | Column Temp | 50° C. |
| | Detector | PDA (220 nm&254 nm) |
| MS | Ionization source | ESI |
| | Drying Gas Flow | 15(L/min) |
| | DL Voltage | 120(v) |
| | Qarray DC Voltage | 20(V) |
| | MS Polarity | Positive |
| | MS Mode | Scan |
| | Mass range | 100-1000 |

### LC-MS Method AB10

AB10

| Instrument | Agilent 1100 LC & Agilent G1956A | | | |
|---|---|---|---|---|
| Software | | Agilent Chemstation Rev. B. 04.03 | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

### LC-MS Method AB25

AB25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10 (L/min) |
| | Nebulizer Pressure | 40 (psi) |
| | Drying Gas | 350° C. |
| | Temperature | |
| | Capillary Voltage | 2500(V) Positive |
| | MS Polarity | Positive |
| | MS Mode | Scan |
| | Mass Range | 100-1500 |

### LC-MS Method AB40

AB40

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 40 | 0.8 |
| | | 0.40 | 40 | 0.8 |
| | | 3.40 | 100 | 0.8 |
| | | 3.90 | 100 | 0.8 |
| | | 3.91 | 40 | 0.8 |
| | | 4.00 | 40 | 1.0 |
| | | 4.50 | 40 | 1.0 |

| | | |
|---|---|---|
| | Post time(min) | 0 |
| | Column Temp | 50° C. |
| | Detector | DAD(Agilent 1100)/ELSD(Agilent 1260 Infinity) |
| MS | Ionization source | ESI |
| | Drying Gas | N₂ |
| | Drying Gas Flow | 10(L/min) |
| | Nebulizer Pressure | 2070(Torr) |
| | Drying Gas | 350° C. |
| | Temperature | |
| | Capillary Voltage | 2500(V) Positive |

### Example 1—Synthesis of Compound I-1: 1-(3-chloro-4-methyl-phenyl)-3-[[2-[1-[2-[(3S)-4-[6-fluoro-7-(2-fluoro-6-hydroxy-phenyl)-1-(2-isopropyl-4-methyl-3-pyridyl)-2-oxo-pyrido[2,3-d]pyrimidin-4-yl]-3-methyl-piperazine-1-carbonyl]allyl]-2,6-dioxo-3-piperidyl]-1-oxo-isoindolin-5-yl]methyl]urea

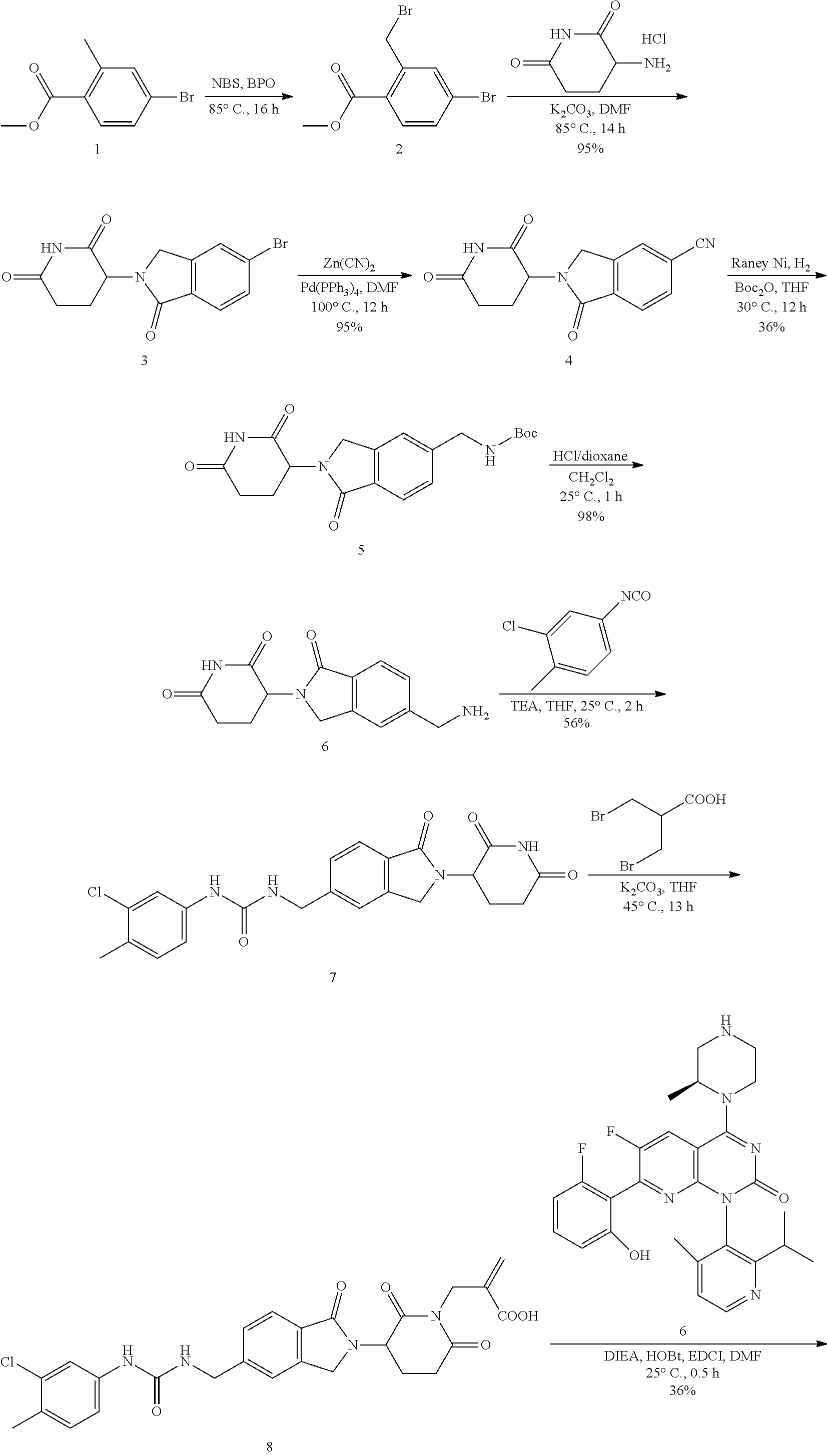

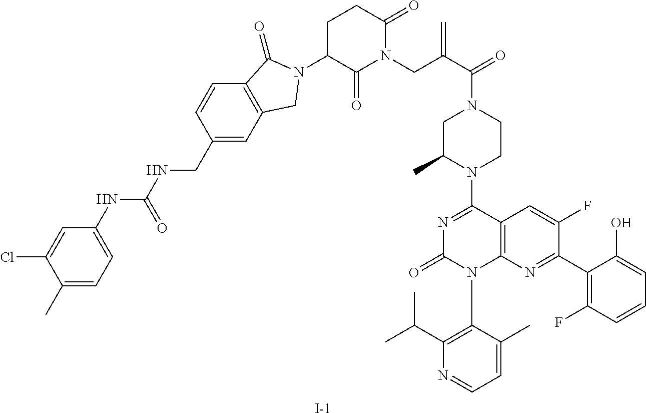 [SMILES: C=C(CN1C(=O)CCC(N2Cc3cc(CNC(=O)Nc4ccc(C)c(Cl)c4)ccc3C2=O)C1=O)C(=O)N1CCN(c2nc(=O)n(-c3c(C)ccnc3C(C)C)c3nc(-c4c(O)cccc4F)c(F)cc23)[C@@H](C)C1 ; [I-]]

### Preparation of compound 2

A mixture of methyl 4-bromo-2-methyl-benzoate (40.5 g, 177 mmol, 1.0 equiv), NBS (31.5 g, 177 mmol, 1.0 equiv) and BPO (4.28 g, 17.7 mmol, 0.1 equiv) in trifluoromethylbenzene (400 mL) was heated to 85° C. and stirred for 16 h. To the reaction mixture was added water (500 mL) and the mixture was extracted with ethyl acetate (500 mL×3). The combined organic phase was washed with brine (500 mL), dried with anhydrous Na₂SO₄, filtered and concentrated in vacuo. The residue was purified by column chromatography on silica gel (Petroleum ether:Ethyl acetate=1:0 to 20:1) to afford methyl 4-bromo-2-(bromomethyl)benzoate (50.0 g, crude) as a white solid. ¹H NMR (400 MHz, CDCl₃): δ 7.84 (d, 1H, J=8.4 Hz), 7.63 (d, 1H, J=2.0 Hz), 7.50 (dd, 1H, J=2.0, 8.4 Hz), 4.89 (s, 2H), 3.94 (s, 3H).

### Preparation of compound 3

A mixture of methyl 4-bromo-2-(bromomethyl)benzoate (4.00 g, 13.0 mmol, 1.0 equiv), DIPEA (6.71 g, 51.9 mmol, 9.05 mL, 4.0 equiv) and 3-aminopiperidine-2,6-dione (4.28 g, 25.9 mmol, 2.0 equiv, HCl salt) in DMF (40 mL) was stirred at 50° C. for 2 h. Then the mixture was heated to 80° C. for another 12 h. The reaction mixture was poured into ice-water (100 mL) and the mixture filtered. The filter cake was washed with EtOAc (50 mL) and dried in vacuo to afford 3-(5-bromo-1-oxo-isoindolin-2-yl)piperidine-2,6-dione (4.00 g, 12.4 mmol, 95% yield) as a gray solid. ¹H NMR (400 MHz, DMSO-d₆): δ 7.88 (1H, s), 7.72-7.65 (m, 2H), 5.12-5.07 (m, 1H), 4.55-4.30 (m, 2H), 2.99-2.82 (m, 1H), 2.67-2.54 (m, 1H), 2.45-2.30 (m, 1H), 2.10-1.90 (m, 1H). LC-MS: MS (ES⁺): RT=0.756 min, m/z=322.9 [M+H⁺]; LC-MS method: AB05.

### Preparation of compound 4

A mixture of 3-(5-bromo-1-oxo-isoindolin-2-yl)piperidine-2,6-dione (6.30 g, 19.5 mmol, 1.0 equiv), Zn(CN)₂ (1.49 g, 12.7 mmol, 804 μL, 0.65 equiv) and Pd(PPh₃)₄(2.25 g, 1.95 mmol, 0.1 equiv) in DMF (50 mL) was heated to 100° C. and stirred for 12 h under N₂. The reaction mixture was diluted with H₂O (30 mL). The precipitate solid was collected by filtration and triturated with ethyl acetate (80 mL). The insoluble material was collected by filtration and dried in vacuo to afford 2-(2,6-dioxo-3-piperidyl)-1-oxo-isoindoline-5-carbonitrile (5.00 g, 18.6 mmol, 95% yield) as a white solid. ¹H NMR (400 MHz, DMSO-d₆): δ 11.03 (s, 1H), 8.17 (s, 1H), 8.03-7.97 (m, 1H), 7.95-7.89 (m, 1H), 5.21-5.11 (m, 1H), 4.60-4.51 (m, 1H), 4.48-4.39 (m, 1H), 2.99-2.90 (m, 1H), 2.66-2.57 (m, 1H), 2.46-2.36 (m, 1H), 2.09-1.98 (m, 1H).

### Preparation of compound 5

To a mixture of 2-(2,6-dioxo-3-piperidyl)-1-oxo-isoindoline-5-carbonitrile (12.0 g, 44.6 mmol, 1.0 equiv) in DMF (20 mL) and THF (20 mL) was added Boc₂O (15.6 g, 71.3 mmol, 16.4 mL, 1.6 equiv) and Raney Ni (4.20 g, 49.0 mmol, 1.1 equiv). The mixture was degassed and stirred at 30° C. for 12 h under H₂ (50 psi). The reaction mixture was filtered and the filtrate was concentrated in vacuo. The residue was triturated with ethyl acetate (80 mL). The insoluble material was collected by filtration and dried in vacuo. The residue was purified by prep-HPLC (column: Kromasil Eternity XT 250*80 mm*10 um; mobile phase: [water (0.225% FA)-ACN]; B %: 25%-45%, 15 min) to afford tert-butyl N-[[2-(2,6-dioxo-3-piperidyl)-1-oxo-isoindolin-5-yl]methyl]carbamate (6.00 g, 16.1 mmol, 36% yield) as a white solid. ¹H NMR (400 MHz, DMSO-d₆): δ 10.98 (s, 1H), 7.72-7.63 (m, 1H), 7.55-7.34 (m, 3H), 5.17-5.04 (m, 1H), 4.50-4.38 (m, 1H), 4.35-4.27 (m, 1H), 4.26-5.14 (m, 2H), 2.99-2.83 (m, 1H), 2.68-2.55 (m, 1H), 2.45-2.29 (m, 1H), 2.06-1.94 (m, 1H), 1.40 (s, 9H).

### Preparation of compound 6

A mixture of tert-butyl N-[[2-(2,6-dioxo-3-piperidyl)-1-oxo-isoindolin-5-yl]methyl]carbamate (6.00 g, 16.1 mmol, 1.0 equiv) in CH₂Cl₂ (50 mL) and 4 M HCl/dioxane (50 mL) was stirred at 25° C. for 1 h. The reaction mixture was filtered and the filtrate was concentrated in vacuo to afford 3-[5-(aminomethyl)-1-oxo-isoindolin-2-yl]piperidine-2,6-dione (4.90 g, 15.8 mmol, 98% yield, HCl salt) as a white solid. ¹H NMR (400 MHz, DMSO-d₆): δ 11.00 (s, 1H), 8.62 (s, 3H), 7.83-7.71 (m, 2H), 7.69-7.58 (m, 1H), 5.18-5.09 (m, 1H), 4.53-4.44 (m, 1H), 4.40-4.30 (m, 1H), 4.20-4.09 (m, 2H), 3.01-2.86 (m, 1H), 2.68-2.57 (m, 1H), 2.46-2.35 (m, 1H), 2.08-1.95 (m, 1H), 2.08-1.95 (m, 1H).

### Preparation of compound 7

To a solution of 3-[5-(aminomethyl)-1-oxo-isoindolin-2-yl]piperidine-2,6-dione (500 mg, 1.61 mmol, 1.0 equiv, HCl salt) and TEA (490 mg, 4.84 mmol, 3.0 equiv) in THF (10 mL) was added 2-chloro-4-isocyanato-1-methyl-benzene (325 mg, 1.94 mmol, 1.2 equiv) at 0° C. The mixture was stirred at 25° C. for 2 h and quenched by 0.1 mL water. The mixture was concentrated and the residue was purified by prep-HPLC (column: Waters Xbridge C18 150*50 mm*10 um; mobile phase: [water(10 mM NH₄HCO₃)-ACN]; B %: 23%-53%, 11 min) to afford 1-(3-chloro-4-methyl-phenyl)-3-[[2-(2,6-dioxo-3-piperidyl)-1-oxo-isoindolin-5-yl]methyl]urea (400 mg, 56% yield) as a white solid. LC-MS: MS (ES⁺): RT=0.574 min, m/z=441.2 [M+H⁺]; LC-MS method: AB05.

### Preparation of compound 8

To a solution of 1-(3-chloro-4-methyl-phenyl)-3-[[2-(2,6-dioxo-3-piperidyl)-1-oxo-isoindolin-5-yl]methyl]urea (350 mg, 794 μmol, 1.0 equiv) in DMF (7 mL) was added K₂CO₃ (329 mg, 2.38 mmol, 3.0 equiv). The mixture was stirred at 45° C. for 1 h and a solution of 3-bromo-2-(bromomethyl)propanoic acid (293 mg, 1.19 mmol, 1.5 equiv) in DMF (3.5 mL) was added dropwise. The mixture was stirred at 45° C. for 12 h and the pH was adjusted to 6-7 by TFA. The residue was purified by prep-HPLC (column: Phenomenex luna C18 150*40 mm*15 um; mobile phase: [water (0.1% TFA)-ACN]; B %: 28%-58%, 11 min) to afford 2-[[3-[5-[[(3-chloro-4-methyl-phenyl)carbamoylamino]methyl]-1-oxo-isoindolin-2-yl]-2,6-dioxo-1-piperidyl]methyl]prop-2-enoic acid (105 mg, 25% yield) as a white solid. ¹H NMR (400 MHz, DMSO-d₆) δ 12.8 (s, 1H), 8.78 (s, 1H), 7.79-7.62 (m, 2H), 7.51 (s, 1H), 7.44 (d, J=7.7 Hz, 1H), 7.22-7.08 (m, 2H), 6.81 (t, J=5.8 Hz, 1H), 6.05 (s, 1H), 5.49 (s, 1H), 5.30 (m, 1H), 4.55-4.20 (m, 6H), 3.18-3.00 (m, 1H), 2.80 (m, 1H), 2.47-2.33 (m, 1H), 2.23 (s, 3H), 2.13-1.98 (m, 1H). LC-MS: MS (ES⁺): RT=0.601 min, m/z=525.3 [M+H⁺]; LC-MS method: AB05.

### Preparation of Compound I-1

To a solution of 6-fluoro-7-(2-fluoro-6-hydroxy-phenyl)-1-(2-isopropyl-4-methyl-3-pyridyl)-4-[(2S)-2-methylpiperazin-1-yl]pyrido[2,3-d]pyrimidin-2-one (24 mg, 48 μmol, 1.0 equiv) and 2-[[3-[5-[[(3-chloro-4-methyl-phenyl)carbamoylamino]methyl]-1-oxo-isoindolin-2-yl]-2,6-dioxo-1-piperidyl]methyl]prop-2-enoic acid (25 mg, 48 μmol, 1.0 equiv) in DMF (2 mL) was added DIEA (12 mg, 95 μmol, 2.0 equiv), EDCI (11 mg, 57 μmol, 1.2 equiv) and HOBt (8 mg, 57 μmol, 1.2 equiv). The mixture was stirred at 25° C. for 0.5 h and quenched by 0.1 mL water. The residue was purified by prep-HPLC (column: Phenomenex Gemini-NX C18 75*30 mm*3 μm; mobile phase: [water (0.225% FA)-ACN]; B %: 35%-65%, 7 min) to afford 1-(3-chloro-4-methyl-phenyl)-3-[[2-[1-[2-[(3S)-4-[6-fluoro-7-(2-fluoro-6-hydroxy-phenyl)-1-(2-isopropyl-4-methyl-3-pyridyl)-2-oxo-pyrido[2,3-d]pyrimidin-4-yl]-3-methyl-piperazine-1-carbonyl]allyl]-2,6-dioxo-3-piperidyl]-1-oxo-isoindolin-5-yl]methyl]urea (18 mg, 36% yield, FA salt) as a light yellow solid. ¹H NMR: (400 MHz, CD₃OD) δ 8.39 (d, J=5.0 Hz, 1H), 8.24-8.11 (m, 1H), 7.77 (d, J=7.7 Hz, 1H), 7.57 (s, 1H), 7.54-7.43 (m, 2H), 7.30-7.19 (m, 2H), 7.16-7.04 (m, 2H), 6.73-6.51 (m, 2H), 5.57-5.41 (m, 1H), 5.38-5.27 (m, 1H), 5.26-5.12 (m, 1H), 4.74-4.23 (m, 8H), 4.17-3.38 (m, 3H), 3.11-2.89 (m, 2H), 2.88-2.72 (m, 1H), 2.63-2.42 (m, 1H), 2.33-2.11 (m, 4H), 2.01 (d, J=8.3 Hz, 3H), 1.62-1.26 (m, 3H), 1.18 (m, 3H), 1.09-0.90 (m, 3H). LC-MS: MS (ES⁺): RT=2.032 min, m/z=1013.3 [M+H⁺]; LC-MS method: AB25.

### Example 2—Synthesis of Compound I-2: 1-(3-chloro-4-methyl-phenyl)-3-[[2-[1-[2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carbonyl]allyl]-2,6-dioxo-3-piperidyl]-1-oxo-isoindolin-5-yl]methyl]urea

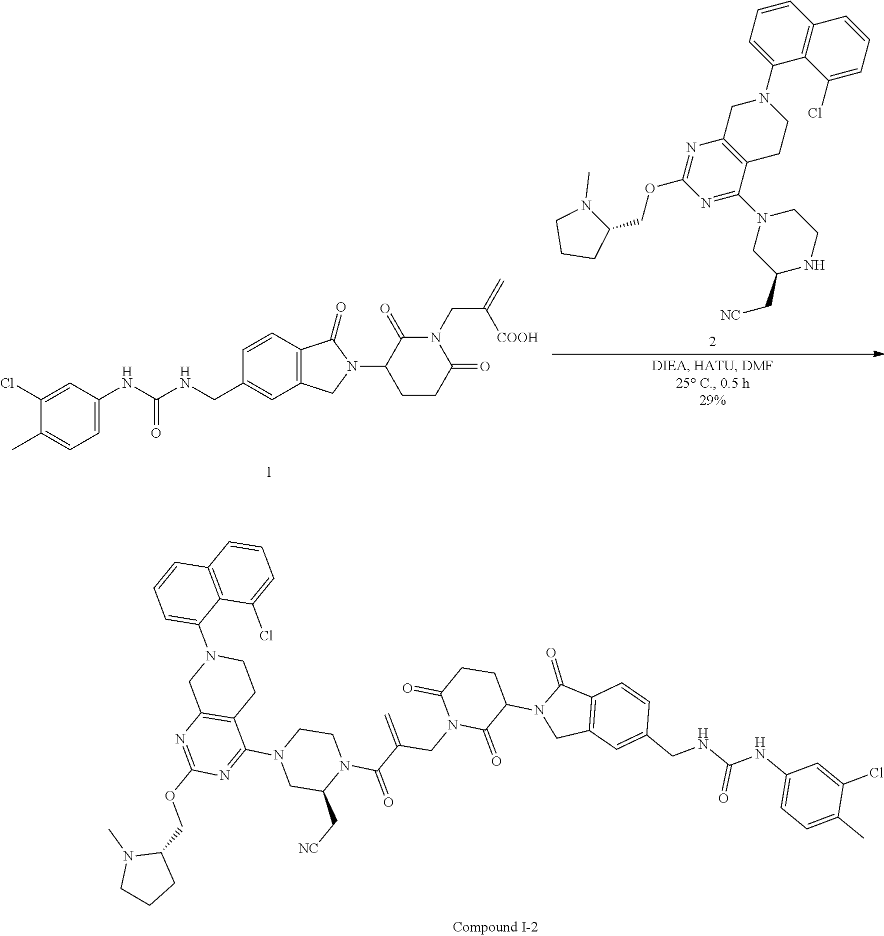 [SMILES: C=C(CN1C(=O)CCC(N2Cc3cc(CNC(=O)Nc4ccc(C)c(Cl)c4)ccc3C2=O)C1=O)C(=O)O ; CN1CCC[C@H]1COc1nc2c(c(N3CCN[C@@H](CC#N)C3)n1)CCN(c1cccc3cccc(Cl)c13)C2 ; C=C(CN1C(=O)CCC(N2Cc3cc(CNC(=O)Nc4ccc(C)c(Cl)c4)ccc3C2=O)C1=O)C(=O)N1CCN(c2nc(OC[C@@H]3CCCN3C)nc3c2CCN(c2cccc4cccc(Cl)c24)C3)C[C@@H]1CC#N]

### Preparation of Compound I-2

To a solution of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (30 mg, 57 μmol, 1.0 equiv; which can be prepared as described in J. Med. Chem., 2020, 63, 6679-6693) and 2-[[3-[5-[[(3-chloro-4-methyl-phenyl)carbamoylamino]methyl]-1-oxo-isoindolin-2-yl]-2,6-dioxo-1-piperidyl]methyl]prop-2-enoic acid (30 mg, 57 μmol, 1.0 equiv; prepared as described above) in DMF (2 mL) was added DIEA (15 mg, 0.11 mmol, 2.0 equiv) and HATU (22 mg, 57 umol, 1.0 equiv). The mixture was stirred at 25° C. for 0.5 h and quenched by 0.1 mL water. The mixture was purified by prep-HPLC (column: Phenomenex Gemini-NX C18 75*30 mm*3 um; mobile phase: [water (0.225% FA)-ACN]; B %: 35%-45%, 7 min) to afford 1-(3-chloro-4-methyl-phenyl)-3-[[2-[1-[2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carbonyl]allyl]-2,6-dioxo-3-piperidyl]-1-oxo-isoindolin-5-yl]methyl]urea (17 mg, 29% yield) as a light yellow solid. ¹H NMR: (400 MHz, CD₃OD) δ 8.48 (s, 1H), 7.83 (d, J=7.8 Hz, 1H), 7.78-7.72 (m, 1H), 7.71-7.63 (m, 1H), 7.59-7.43 (m, 5H), 7.41-7.27 (m, 2H), 7.19-7.04 (m, 2H), 5.55-5.28 (m, 2H), 5.25-5.11 (m, 1H), 4.77-3.87 (m, 13H), 3.78-3.40 (m, 6H), 3.24-2.72 (m, 11H), 2.71-2.40 (m, 2H), 2.36-2.23 (m, 4H), 2.23-1.81 (m, 4H). LC-MS: MS (ES⁺): RT=2.248 min, m/z=1038.3 [M+H⁺]; LC-MS method: AB25.

### Example 3—Synthesis of Compound I-3: 1-[[2-[1-[2-[(3R)-3-[4-amino-3-(4-phenoxyphenyl)pyrazolo[3,4-d]pyrimidin-1-yl]piperidine-1-carbonyl]allyl]-2,6-dioxo-3-piperidyl]-1-oxo-isoindolin-5-yl]methyl]-3-(3-chloro-4-methyl-phenyl)urea

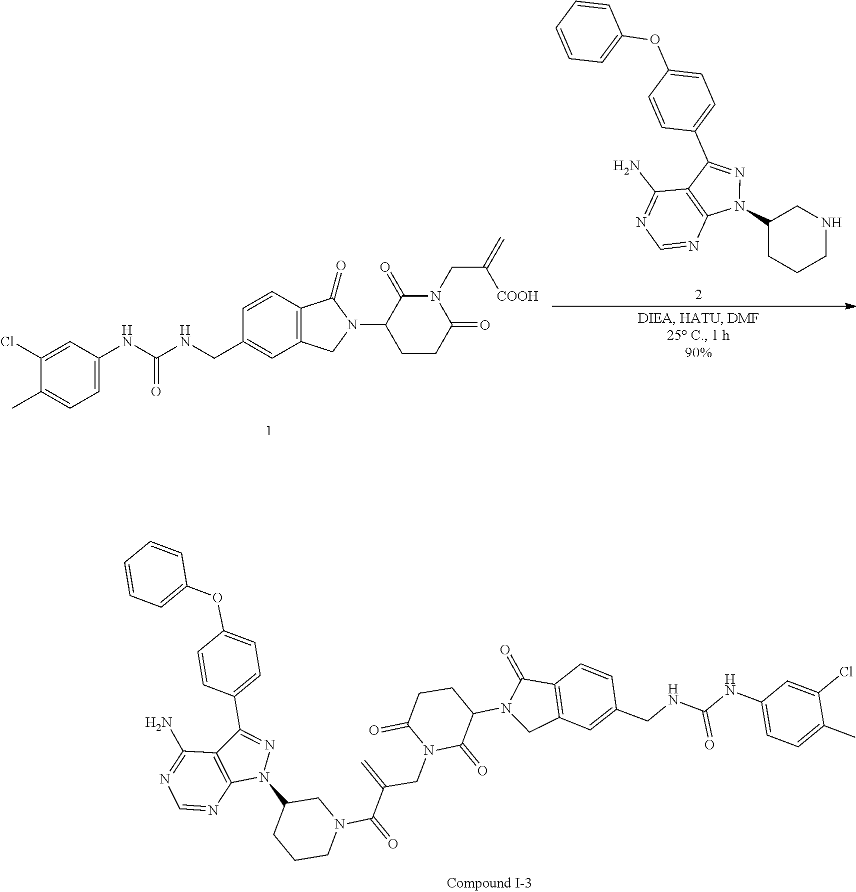

### Preparation of Compound I-3

To a solution of 3-(4-phenoxyphenyl)-1-[(3R)-3-piperidyl]pyrazolo[3,4-d]pyrimidin-4-amine (18 mg, 48 umol, 1.0 equiv; which can be prepared as described in Organic & Biomolecular Chemistry (2015), 13(18), 5147-5157) and 2-[[3-[5-[[(3-chloro-4-methyl-phenyl)carbamoylamino]methyl]-1-oxo-isoindolin-2-yl]-2,6-dioxo-1-piperidyl]methyl]prop-2-enoic acid (25 mg, 48 μmol, 1.0 equiv; prepared as described above) in DMF (2 mL) was added DIEA (12 mg, 95 umol, 2.0 equiv) and HATU (22 mg, 57 μmol, 1.2 equiv). The mixture was stirred at 25° C. for 0.5 h and quenched by 0.1 mL water. The residue was purified by prep-HPLC (column: Phenomenex Gemini-NX C18 75*30 mm*3 μm; mobile phase: [water (0.225% FA)-ACN]; B %: 45%-75%, 7 min) to afford 1-[[2-[1-[2-[(3R)-3-[4-amino-3-(4-phenoxyphenyl)pyrazolo [3,4-d]pyrimidin-1-yl]piperidine-1-carbonyl]allyl]-2,6-dioxo-3-piperidyl]-1-oxo-isoindolin-5-yl]methyl]-3-(3-chloro-4-methyl-phenyl)urea (13 mg, 29% yield, FA salt) as a light yellow solid. ¹H NMR: (400 MHz, CD₃OD) δ 7.84-7.32 (m, 8H), 7.23-7.01 (m, 8H), 5.41-5.12 (m, 3H), 4.71-3.87 (m, 10H), 3.82-3.44 (m, 1H), 3.08-2.81 (m, 3H), 2.60-2.37 (m, 1H), 2.37-2.24 (m, 5H), 2.21-1.96 (m, 3H). LC-MS: MS (ES⁺): RT=2.303 min, m/z=893.3 [M+H⁺]; LC-MS method: AB25.

### Example 4—Synthesis of Compound 1-(5-chloro-2-hydroxy-phenyl)-3-[[2-(2,6-dioxo-3-piperidyl)-1-oxo-isoindolin-5-yl]methyl]urea

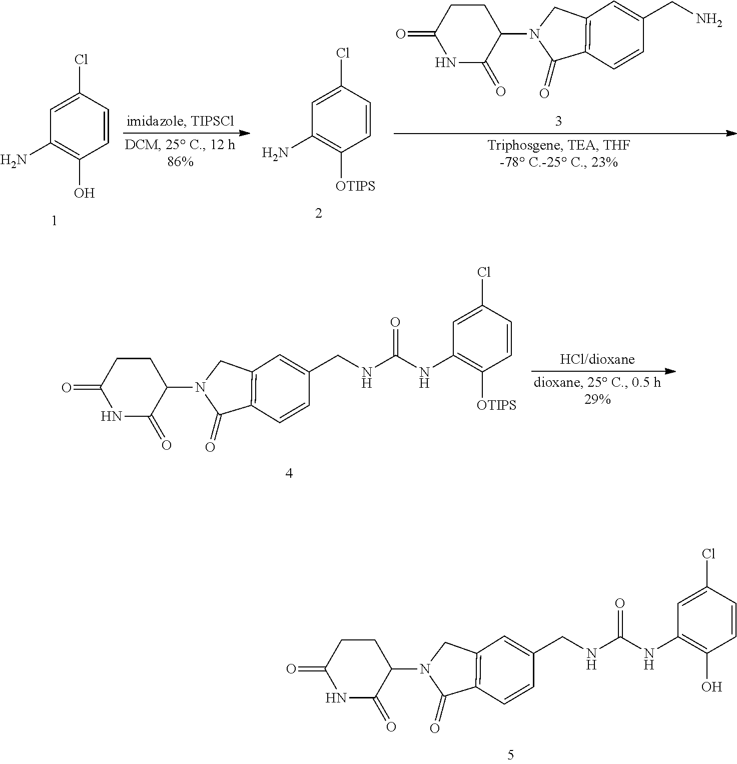

### Preparation of compound 2

To a solution of 2-amino-4-chloro-phenol (1.0 g, 7 mmol, 1.0 equiv) in DCM (15 mL) was added imidazole (1.42 g, 21 mmol, 3.0 equiv) and TIPSCI (2.0 g, 10.5 mmol, 1.5 equiv). The mixture was stirred at 25° C. for 12 h. The mixture was added water (50 mL) and extracted with EtOAc (3×50 mL). The combined organic phase was washed with brine (50 mL), dried over anhydrous Na₂SO₄, filtered and concentrated. The residue was purified by column chromatography on silica gel (petroleum ether: ethyl acetate=10:1 to 2:1) to give 5-chloro-2-triisopropylsilyloxy-aniline (1.8 g, 86% yield) as a yellow solid. ¹H NMR (400 MHz, CDCl₃) δ 6.93-6.81 (m, 1H), 6.73-6.58 (m, 2H), 1.36-1.27 (m, 3H), 1.13-1.12 (m, 6H), 1.11-1.10 (m, 6H), 1.06 (s, 6H). LC-MS: MS (ES⁺): RT=1.147 min, m/z=300.2 [M+H⁺]; LC-MS method: AB05.

### Preparation of compound 4

To a solution of TEA (506 mg, 5 mmol, 5.0 equiv) and triphosgene (237 mg, 800 mol, 0.8 equiv) in THF (8 mL) was added 5-chloro-2-triisopropylsilyloxy-aniline (300 mg, 1 mmol, 1.0 equiv) in THF (2 mL) at −78° C. and the mixture was stirred for 0.5 h. 3-[5-(aminomethyl)-1-oxo-isoindolin-2-yl]piperidine-2,6-dione (372 mg, 1.2 mmol, 1.2 equiv, HCl salt; prepared as described above) was added. The mixture was slowly warmed to 25° C. and stirred for 2 h. The mixture was added water (50 mL) and extracted with EtOAc (3×50 mL). The combined organic phase was washed with brine (50 mL), dried over anhydrous Na₂SO₄, filtered and concentrated. The residue was purified by column chromatography on silica gel (petroleum ether: ethyl acetate=1:1 to 0:1) to give 1-(5-chloro-2-triisopropylsilyloxy-phenyl)-3-[[2-(2,6-dioxo-3-piperidyl)-1-oxo-isoindolin-5-yl]methyl]urea (140 mg, 23% yield) as a white solid. LC-MS: MS (ES⁺): RT=0.985 min, m/z=599.1 [M+H⁺]; LC-MS method: AB05.

### Preparation of 1-(5-chloro-2-hydroxy-phenyl)-3-[[2-(2,6-dioxo-3-piperidyl)-1-oxo-isoindolin-5-yl]methyl]urea

To a solution of 1-(5-chloro-2-triisopropylsilyloxy-phenyl)-3-[[2-(2,6-dioxo-3-piperidyl)-1-oxo-isoindolin-5-yl]methyl]urea (70 mg, 117 μmol, 1.0 equiv) in dioxane (1.5 mL) was added HCl/dioxane (1 mL). The mixture was stirred at 25° C. for 0.5 h and then concentrated. The resulting residue was purified by prep-HPLC (column: YMC Triart 30*150 mm*7 um; mobile phase: [water (HCl)-ACN]; B %: 14%-34%, 9 min) to give 1-(5-chloro-2-hydroxy-phenyl)-3-[[2-(2,6-dioxo-3-piperidyl)-1-oxo-isoindolin-5-yl]methyl]urea (15 mg, 29% yield) as a white solid. ¹H NMR (400 MHz, DMSO-d₆) δ 10.23-10.07 (m, 1H), 9.34 (s, 1H), 7.39-7.24 (m, 2H), 6.91-6.55 (m, 4H), 6.01-5.88 (m, 2H), 4.35-4.20 (m, 1H), 3.68-3.44 (m, 4H), 2.16-2.01 (m, 1H), 1.85-1.74 (m, 1H), 1.59-1.47 (m, 1H), 1.26-1.11 (m, 1H). LC-MS: MS (ES⁺): RT=2.324 min, m/z=443.0 [M+H⁺]; LC-MS method: ABO1.

### Example 5—Synthesis of Compound 1-(3-chloro-5-hydroxy-phenyl)-3-[[2-(2,6-dioxo-3-piperidyl)-1-oxo-isoindolin-5-yl]methyl]urea

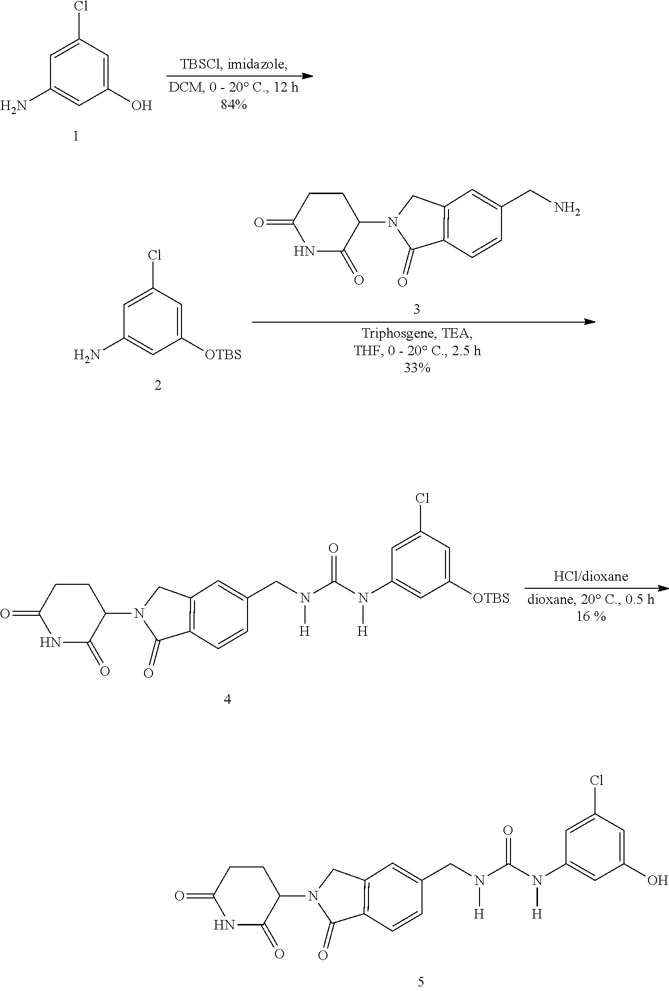 [SMILES: Nc1cc(O)cc(Cl)c1 ; CC(C)(C)[Si](C)(C)Oc1cc(N)cc(Cl)c1 ; NCc1ccc2c(c1)CN(C1CCC(=O)NC1=O)C2=O ; CC(C)(C)[Si](C)(C)Oc1cc(Cl)cc(NC(=O)NCc2ccc3c(c2)CN(C2CCC(=O)NC2=O)C3=O)c1 ; O=C1CCC(N2Cc3cc(CNC(=O)Nc4cc(O)cc(Cl)c4)ccc3C2=O)C(=O)N1]

### Preparation of compound 2

To a mixture of 3-amino-5-chloro-phenol (0.5 g, 3 mmol, 1 equiv) in DCM (10 mL) was added imidazole (711 mg, 10 mmol, 3 equiv) and TBSCI (787 mg, 5 mmol, 1.5 equiv) at 0° C. The mixture was stirred at 20° C. for 12 h. The residue was purified by column separation (SiO₂, Petroleum ether:Ethyl acetate=20:1 to 10:1) to give 3-[tert-butyl (dimethyl)silyl]oxy-5-chloro-aniline (750 mg, 84% yield) as a yellow oil. LC-MS: MS (ES⁺): RT=0.920 min, m/z=258.0 [M+H]⁺; LC-MS method: AB05.

### Preparation of compound 4

To a solution of TEA (588 mg, 5 mmol, 5 equiv) and triphosgene (276 mg, 930 mol, 0.8 equiv) in THF (8 mL) was added 3-[tert-butyl(dimethyl)silyl]oxy-5-chloro-aniline (300 mg, 1 mmol, 1 equiv) in THF (2 mL) at −78° C. and the mixture was stirred for 0.5 h. Then 3-[5-(aminomethyl)-1-oxo-isoindolin-2-yl]piperidine-2,6-dione (432 mg, 1 mmol, 1.2 equiv, HCl salt; prepared as described above) was added. The mixture was slowly warmed to 25° C. and stirred for 2 h. The mixture was quenched with water (50 mL) and extracted with EtOAc (3×50 mL). The combined organic phase was washed with brine (50 mL), dried over anhydrous Na₂SO₄, filtered and concentrated. The residue was purified by column chromatography on silica gel (petroleum ether: ethyl acetate=1:1 to 0:1) to give 3-[tert-butyldimethyl)silyl]oxy-5-chloro-phenyl]-3-[[2-(2, 6-dioxo-3-piperidyl)-1-oxo-isoindolin-5-yl]methyl] urea (210 mg, 33% yield) as a yellow solid. LC-MS: MS (ES⁺): RT=0.923 m/z=557.1 [M+H]⁺; LC-MS method: AB05.

### Preparation of 1-(3-chloro-5-hydroxy-phenyl)-3-[[2-(2, 6-dioxo-3-piperidyl)-1-oxo-isoindolin-5-yl] methyl] urea

To a solution of 1-[3-[tert-butyl(dimethyl)silyl]oxy-5-chloro-phenyl]-3-[[2-(2,6-dioxo-3-piperidyl)-1-oxo-isoindolin-5-yl]methyl]urea (100 mg, 179 μmol, 1 equiv) in dioxane (2 mL) was added HCl/dioxane (1 mL). The mixture was stirred at 20° C. for 0.5 h and then concentrated. The resulting residue was purified by prep-HPLC (YMC Triart 30*150 mm*7 m; mobile phase: [water (HCl)-ACN]; B %: 20%-40%, 9 min) to give 1-(3-chloro-5-hydroxy-phenyl)-3-[[2-(2,6-dioxo-3-piperidyl)-1-oxo-isoindolin-5-yl]methyl]urea (14 mg, 16% yield) as an off-white solid. ¹H NMR (400 MHz, CD₃OD): δ7.83-7.70 (m, 1H), 7.57 (s, 1H), 7.47 (d, J=2.4 Hz, 1H), 6.95 (d, J=15.2 Hz, 1H), 6.80 (s, 1H), 6.46-6.37 (m, 1H), 5.18-5.09 (m, 1H), 4.56-4.42 (m, 4H), 2.96-2.84 (m, 1H), 2.83-2.71 (m, 1H), 2.57-2.40 (m, 1H), 2.23-2.12 (m, 1H). LC-MS: MS (ES⁺): RT=2.233 min, m/z=443.1[M+H⁺]; LC-MS method: ABO1.

### Example 6—Synthesis of Compound 1-(3-chloro-4-hydroxyphenyl)-3-((2-(2,6-dioxopiperidin-3-yl)-1-oxoisoindolin-5-yl)methyl)urea

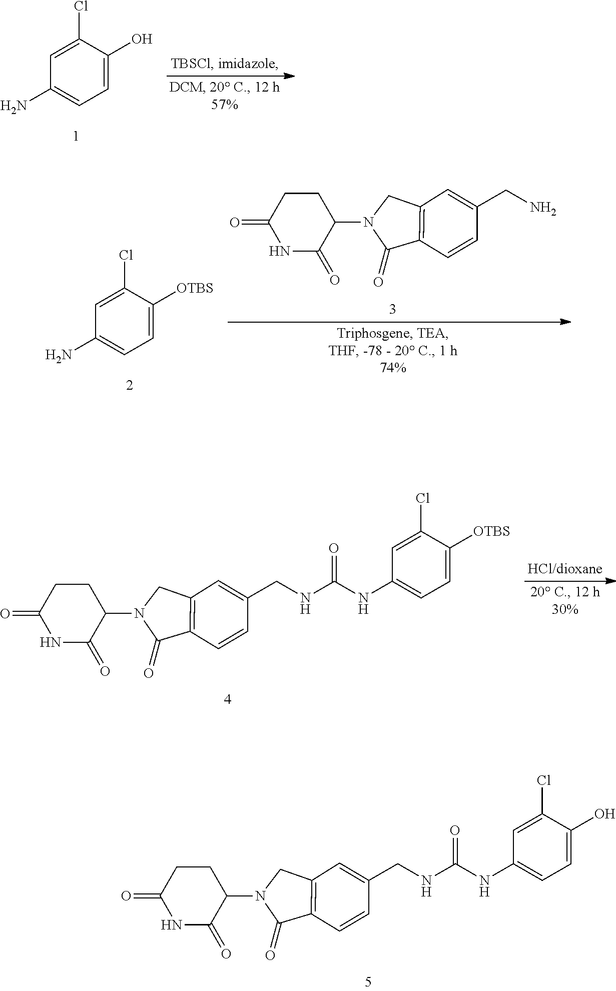 [SMILES: Nc1ccc(O)c(Cl)c1 ; CC(C)(C)[Si](C)(C)Oc1ccc(N)cc1Cl ; NCc1ccc2c(c1)CN(C1CCC(=O)NC1=O)C2=O ; CC(C)(C)[Si](C)(C)Oc1ccc(NC(=O)NCc2ccc3c(c2)CN(C2CCC(=O)NC2=O)C3=O)cc1Cl ; O=C1CCC(N2Cc3cc(CNC(=O)Nc4ccc(O)c(Cl)c4)ccc3C2=O)C(=O)N1]

### Preparation of compound 2

To a solution of 4-amino-2-chlorophenol (1.0 g, 7 mmol, 1.0 equiv) and imidazole (1.66 g, 24 mmol, 3.5 equiv) in DCM (25 mL) was added TBSCI (1.57 g, 10 mmol, 1.5 equiv) at 0° C. The mixture was stirred at 20° C. for 12 h. The mixture was concentrated and the residue was purified by column chromatography (SiO₂, Petroleum ether/Ethyl acetate=0/1 to 20/1) to give 4-((tert-butyldimethylsilyl)oxy)-3-chloroaniline (1.06 g, 57% yield) was obtained as a brown oil. ¹H NMR (400 MHz, CDCl₃): δ 6.78 (d, J=2.8 Hz, 1H), 6.72 (d, J=8.6 Hz, 1H), 6.54 (dd, J=2.8, 8.6 Hz, 1H), 1.02 (s, 10H), 0.19 (s, 6H). LC-MS: MS (ES⁺): RT=0.919 min, m/z=258.0 [M+H]⁺; LC-MS method: AB05.

### Preparation of compound 4

To a solution of bis(trichloromethyl) carbonate (97 mg, 326 μmol, 0.56 equiv) in THF (10 mL) was added TEA (588 mg, 5.8 mmol, 10.0 equiv) and 4-((tert-butyldimethylsilyl)oxy)-3-chloroaniline (150 mg, 581 μmol, 1.0 equiv) in THF (5 mL) at −78° C., and the mixture was stirred for 0.5 h. 3-(5-(aminomethyl)-1-oxoisoindolin-2-yl)piperidine-2,6-dione (180 mg, 581 umol, 1.0 equiv, HCl salt; prepared as described above) was added at −78° C. and the mixture was stirred at 20° C. for 0.5 h. The mixture was poured into aq. NaHCO₃ (10 mL) at 0° C. and then extracted with EtOAc (3×10 mL). The combined organic layers were washed with brine (10 mL), dried over Na₂SO₄, filtered and concentrated. The resulting residue was purified by column chromatography (SiO₂, Petroleum ether/Ethyl acetate=1/1 to Ethyl acetate/Methanol=10/1) to give 1-(4-((tert-butyldimethylsilyl)oxy)-3-chlorophenyl)-3-((2-(2,6-dioxopiperidin-3-yl)-1-oxoisoindolin-5-yl)methyl)urea (240 mg, 74% yield) as a brown solid. LC-MS: MS (ES⁺): RT=1.017 min, m/z=557.1 [M+H]⁺; LC-MS method: AB05.

### Preparation of 1-(3-chloro-4-hydroxyphenyl)-3-((2-(2,6-dioxopiperidin-3-yl)-1-oxoisoindolin-5-yl)methyl)urea

To a solution of 1-(4-((tert-butyldimethylsilyl)oxy)-3-chlorophenyl)-3-((2-(2,6-dioxopiperidin-3-yl)-1-oxoisoindolin-5-yl)methyl)urea (120 mg, 215 μmol, 1.0 equiv) in dioxane (2 mL) was added HCl/dioxane (4 mL). The mixture was stirred at 20° C. for 12 h. The reaction mixture was concentrated and the residue was purified by prep-HPLC (column: YMC Triart 30*150 mm*7 m; mobile phase: [water (HCl)-ACN]; B %: 14%-34%, 9 min) to give 1-(3-chloro-4-hydroxyphenyl)-3-((2-(2,6-dioxopiperidin-3-yl)-1-oxoisoindolin-5-yl)methyl)urea (28 mg, 65 μmol, 30% yield, 100% purity) as a off-white solid. ¹H NMR (400 MHz, DMSO-d₆): δ 10.97 (s, 1H), 9.65 (s, 1H), 8.71-8.49 (s, 1H), 7.68 (d, J=7.9 Hz, 1H), 7.52 (d, J=10.8 Hz, 2H), 7.46-7.37 (m, 1H), 7.09-6.97 (d, J=7.9 Hz, 1H), 6.83 (d, J=8.3 Hz, 1H), 6.74 (m, 1H), 5.20-5.02 (m, 1H), 4.47-4.25 (m, 4H), 2.95-2.86 (m, 1H), 2.67-2.69 (m, 1H), 2.39-2.33 (m, 1H), 2.05-1.94 (m, 1H). LC-MS: MS (ES⁺): RT=1.739 min, m/z=443.1 [M+H]⁺; LC-MS method: AB10.

### Example 7—Synthesis of Additional Compounds

The following compounds were prepared using procedures analogous to those described above.

| | | | | Retention | |
|---|---|---|---|---|---|
| | | MW | Observed | Time | LC-MS |
| No. | Structure | Exact | Mass | (min) | Method |
| I-4 | 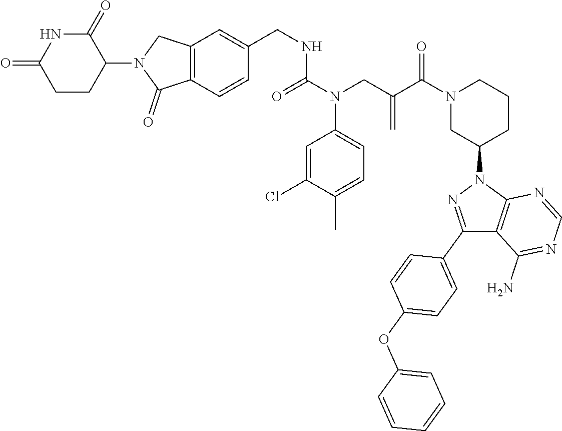 [SMILES: C=C(CN(C(=O)NCc1ccc2c(c1)CN(C1CCC(=O)NC1=O)C2=O)c1ccc(C)c(Cl)c1)C(=O)N1CCC[C@@H](n2nc(-c3ccc(Oc4ccccc4)cc3)c3c(N)ncnc32)C1] | 892.32 | 893.2 | 220- 2.766 | AB05 |
| I-5 | 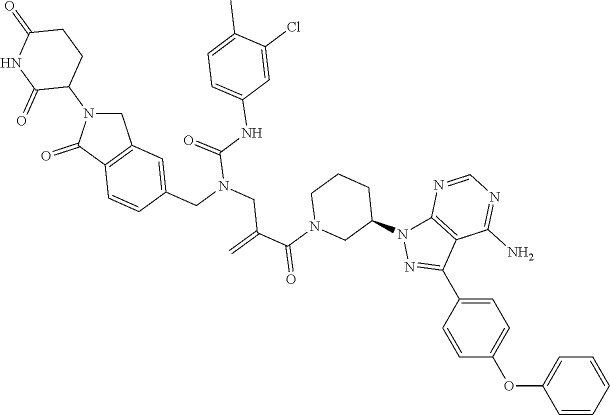 [SMILES: C=C(CN(Cc1ccc2c(c1)CN(C1CCC(=O)NC1=O)C2=O)C(=O)Nc1ccc(C)c(Cl)c1)C(=O)N1CCC[C@@H](n2nc(-c3ccc(Oc4ccccc4)cc3)c3c(N)ncnc32)C1] | 892.32 | 893.2 | 220- 2.782 | AB05 |
| II-8 | 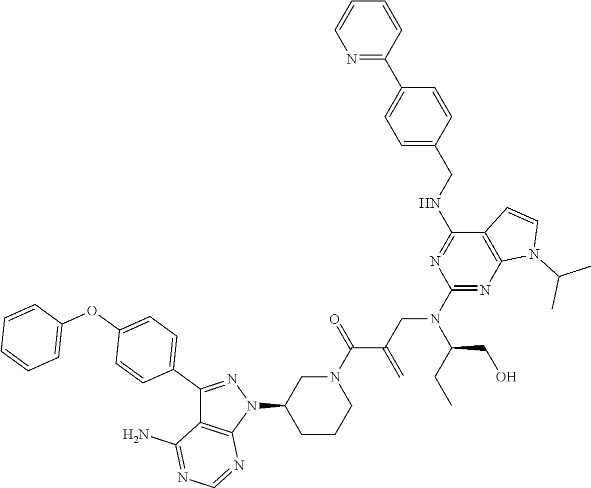 [SMILES: C=C(CN(c1nc(NCc2ccc(-c3ccccn3)cc2)c2ccn(C(C)C)c2n1)[C@H](CC)CO)C(=O)N1CCC[C@@H](n2nc(-c3ccc(Oc4ccccc4)cc3)c3c(N)ncnc32)C1] | 882.44 | 442.7, 884.4 | 220- 2.009 | AB25 |

### Example 8—Preparation of Synthetic Intermediate Compounds

Procedures for the synthesis of various compounds used herein as synthetic intermediates is provided below.

### Compound A: (R)-2-(3-(4-amino-3-(4-phenoxyphenyl)-1H-pyrazolo[3,4-d]pyrimidin-1-yl)piperidine-1-carbonyl)allyl (4-nitrophenyl) carbonate

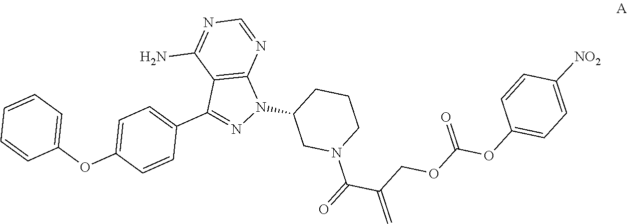 [SMILES: C=C(COC(=O)Oc1ccc([N+](=O)[O-])cc1)C(=O)N1CCC[C@@H](n2nc(-c3ccc(Oc4ccccc4)cc3)c3c(N)ncnc32)C1]

Compound A was prepared according to the procedure described in J. Am. Chem. Soc., 2021, 143, 4979.

### Compound B: 1-(3-(aminomethyl)-5-chlorophenyl)-3-((2-(2,6-dioxopiperidin-3-yl)-1-oxoisoindolin-5-yl)methyl)urea

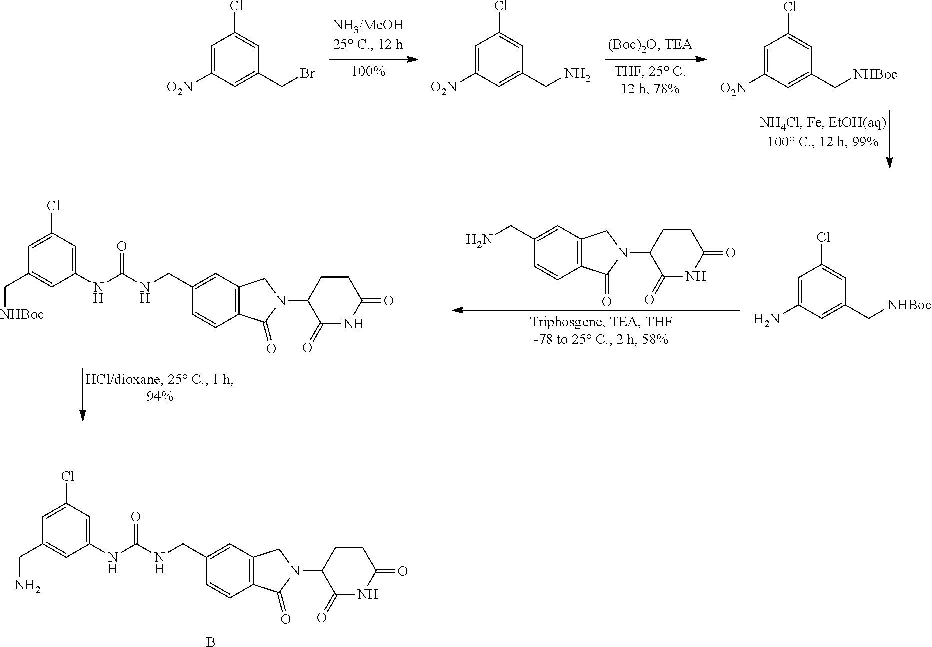

Step 1. A mixture of 1-(bromomethyl)-3-chloro-5-nitro-benzene (900 mg, 3.6 mmol, 1.0 eq) in NH₃/MeOH (5 M, 9 mL) was degassed and purged with N₂ 3 times, then stirred at 25° C. for 12 h under N₂ atmosphere. The mixture was concentrated to get the residue and used for next step without further purification. The (3-chloro-5-nitro-phenyl) methanamine (670 mg, 3.6 mmol, 100% yield) was obtained as a white solid.

Step 2. To a solution of (3-chloro-5-nitro-phenyl)methanamine (670 mg, 3.4 mmol, 1.0 eq) in THF (10 mL) was added (Boc)₂O (1.6 g, 7.2 mmol, 1.7 mL, 2.0 eq) and TEA (1.1 g, 10.8 mmol, 1.5 mL, 3.0 eq). The mixture was stirred at 25° C. for 12 h. The mixture was diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by flash silica gel chromatography (ISCO®; 3 g SepaFlash® Silica Flash Column, Eluent of 0-36% Ethyl acetate/Petroleum ether gradient @60 mL/min) to give the tert-butyl N-[(3-chloro-5-nitro-phenyl) methyl] carbamate (800 mg, 2.8 mmol, 78% yield) as a colorless oil. ¹H NMR: (400 MHz, DMSO-d6) δ=8.15 (s, 1H), 8.07 (s, 1H), 7.77 (s, 1H), 7.65-7.58 (m, 1H), 4.25 (d, J=6.2 Hz, 2H), 1.43-1.38 (m, 9H)

Step 3. To a solution of tert-butyl N-[(3-chloro-5-nitro-phenyl)methyl]carbamate (750 mg, 2.6 mmol, 1.0 eq) in EtOH (9 mL) and H₂O (3 mL) was added NH₄Cl (1.4 g, 26.2 mmol, 10.0 eq), Fe (1.5 g, 26.2 mmol, 10.0 eq). The mixture was stirred at 100° C. for 12 h. The mixture diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by flash silica gel chromatography (ISCO®; 2 g SepaFlash® Silica Flash Column, Eluent of 0-21% Ethyl acetate/Petroleum ether gradient @ 60 mL/min) to give the tert-butyl N-[(3-amino-5-chloro-phenyl) methyl]carbamate (665 mg, 2.6 mmol, 99% yield) as a yellow solid. ¹H NMR: (400 MHz, DMSO-d6) δ=7.42-7.16 (m, 1H), 6.43 (s, 1H), 6.36 (s, 2H), 5.38 (s, 2H), 3.95 (d, J=6.2 Hz, 2H), 1.39 (s, 9H)

Step 4. To a solution of triphosgene (160 mg, 539 μmol, 0.7 eq) in THF (30 mL) stirred at −78° C. under N2 protection, then tert-butyl N-[(3-amino-5-chloro-phenyl)methyl]carbamate (200 mg, 779 μmol, 1.0 eq) and TEA (788 mg, 7.8 mmol, 1.1 mL, 10.0 eq) was added to the mixture and stirred at −78° C. for 30 min. Then the 3-[5-(aminomethyl)-1-oxo-isoindolin-2-yl] piperidine-2, 6-dione (241 mg, 779 μmol, 1.0 eq, HCl) was added to the mixture at −78° C. and stirred for 30 min. Then the mixture was allowed stirred at 25° C. for 1 h under N₂ protection. The mixture was poured into H₂O (100 mL) and extracted with DCM (300 mL×3), then the mixture was washed with brine and concentrated to get the residue. The residue was purified by prep-HPLC (column: Phenomenex C18 250×50 mm×10 um; mobile phase: [water (NH₄HCO₃)-ACN]; B %: 25%-55%, 8 min) to give the tert-butyl N-[[3-chloro-5-[[2-(2,6-dioxo-3-piperidyl)-1-oxo-isoindolin-5-yl]methylcarbamoylamino]phenyl]methyl]carbamate (250 mg, 450 μmol, 58% yield) as a white solid. ¹H NMR: (400 MHz, DMSO-d6) δ=8.91 (s, 1H), 7.72-7.66 (m, 1H), 7.60-7.55 (m, 1H), 7.53-7.49 (m, 1H), 7.47-7.38 (m, 2H), 7.06 (s, 1H), 6.82-6.79 (m, 1H), 5.14-5.06 (m, 1H), 4.43-4.40 (m, 2H), 3.37 (s, 2H), 3.36 (s, 4H), 3.31 (s, 2H), 1.41-1.37 (m, 9H).

Step 5. To a solution of tert-butyl N-[[3-chloro-5-[[2-(2,6-dioxo-3-piperidyl)-1-oxo-isoindolin-5-yl]methylcarbamoylamino]phenyl]methyl]carbamate (250 mg, 450 μmol, 1.0 eq) in HCl/dioxane (20 mL). The mixture was stirred at 25° C. for 1 h. The mixture was concentrated to get the residue. The crude product was triturated with DCM (20 mL) at 25° C. to give the 1-[3-(aminomethyl)-5-chloro-phenyl]-3-[[2-(2,6-dioxo-3-piperidyl)-1-oxo-isoindolin-5-yl]methyl]urea B (220 mg, 421 μmol, 94% yield, 94% purity, HCl) as a white solid. ¹H NMR: (400 MHz, MeOD) δ=7.76 (d, J=8.0 Hz, 1H), 7.56 (s, 1H), 7.53-7.50 (m, 1H), 7.50-7.48 (m, 2H), 7.11-7.08 (m, 1H), 5.18-5.11 (m, 1H), 4.53 (s, 2H), 4.48 (d, J=8.4 Hz, 2H), 4.05 (s, 2H), 2.95-2.74 (m, 2H), 2.55-2.42 (m, 1H), 2.20-2.13 (m, 1H).

### Compound C and Compound Q: 2-(((3R,4R)-4-fluoro-1-(6-((3-methoxy-1-methyl-1H-pyrazol-4-yl)amino)-9-methyl-9H-purin-2-yl)pyrrolidin-3-yl)carbamoyl)allyl (4-nitrophenyl) carbonate (Compound C) and 2-((3R,4R)-3-amino-4-fluoropyrrolidin-1-yl)-N-(3-methoxy-1-methyl-1H-pyrazol-4-yl)-9-methyl-9H-purin-6-amine (Compound Q)

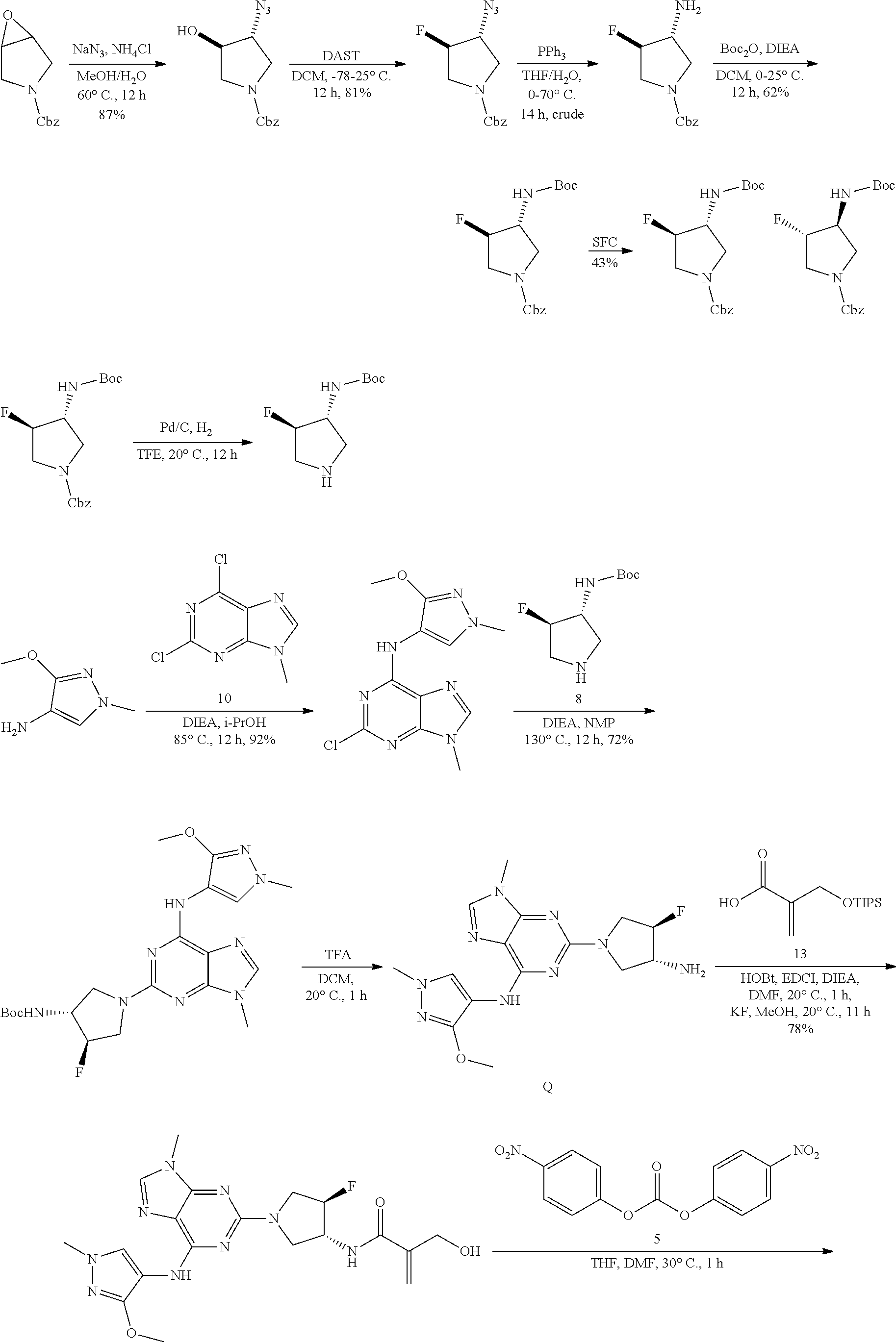

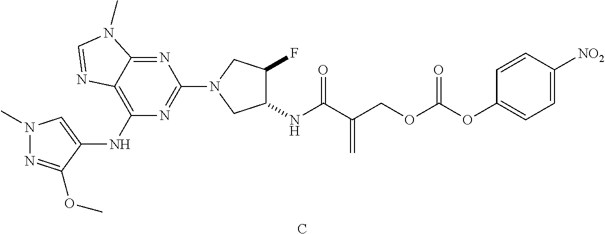

Step 1. To a solution of benzyl 6-oxa-3-azabicyclo [3.1.0] hexane-3-carboxylate (11 g, 50 mmol, 1.0 eq) in MeOH (120 mL) and H₂O (30 mL) was added NaN₃ (4.8 g, 75 mmol, 1.4 eq) and NH₄Cl (2.6 g, 50 mmol, 1.0 eq). The mixture was stirred at 60° C. for 12 h and quenched with NaOH (0.5N, 10 mL). The mixture was concentrated to remove MeOH. The residue was extracted with CH₂Cl₂(3×100 mL) and the combined organic extracts were washed with water, brine, dried over Na₂SO₄, and then concentrated to give benzyl (3R,4R)-3-azido-4-hydroxy-pyrrolidine-1-carboxylate (11.5 g, 87% yield).

Step 2. To a solution of benzyl (3R,4R)-3-azido-4-hydroxy-pyrrolidine-1-carboxylate (22 g, 84 mmol, 1.0 eq) in DCM (160 mL) was added DAST (27 g, 167 mmol, 2.0 eq) in DCM (80 mL) at −78° C., After addition, the mixture was stirred at −78° C. for 1 h and stirred at 20° C. for 11 h. The mixture was poured into saturate aqueous Na₂CO₃ solution (200 mL). The separated organic phase was washed with brine (200 mL), dried (Na₂SO₄) and concentrated. The residue was purified by silica column chromatography on silica gel (Petroleum ether:Ethyl acetate from 10/1 to 4/1) to give benzyl (3R,4R)-3-azido-4-fluoro-pyrrolidine-1-carboxylate (18 g, 81% yield).

Step 3. To a stirred solution of benzyl (3R,4R)-3-azido-4-fluoro-pyrrolidine-1-carboxylate (18 g, 68 mmol, 1.0 eq) in THF (180 mL) was added PPh₃ (22 g, 85 mmol, 1.2 eq) portion wise at 0° C. The resulting mixture was stirred at 25° C. for 2 h and quenched with H₂O (18 mL). The mixture was stirred at 70° C. for 12 h and concentrated. The residue diluted with EtOAc (10 mL) and extracted with HCl (0.5 M, 3×10 mL). The combined organic layer was washed with brine (20 mL), dried (Na₂SO₄) and concentrated to give a benzyl (3R,4R)-3-amino-4-fluoro-pyrrolidine-1-carboxylate (18.0 g).

Step 4. To a solution of benzyl (3R,4R)-3-amino-4-fluoro-pyrrolidine-1-carboxylate (18 g, 75.5 mmol, 1.0 eq) in DCM (300 mL) was added Boc₂O (21 g, 98.2 mmol, 1.3 eq) at 0° C. and DIEA (19 g, 151 mmol, 2.0 eq). The mixture was stirred at 25° C. for 12 h and concentrated. The residue was purified by silica column chromatography on silica gel (Petroleum ether:Ethyl acetate from 50/1 to 5/1) to give benzyl (3R,4R)-3-(tert-butoxycarbonylamino)-4-fluoro-pyrrolidine-1-carboxylate (16 g, 62% yield). ¹H NMR (400 MHz, CDCl3): δ 7.35-7.23 (m, 5H), 5.08-4.87 (m, 3H), 4.45 (s, 1H), 4.27-4.06 (m, 1H), 3.76-3.50 (m, 3H), 3.45-3.31 (m, 1H), 1.37 (s, 9H).

Step 5. Benzyl(3R,4R)-3-(tert-butoxycarbonylamino)-4-fluoro-pyrrolidine-1-carboxylate (16 g, 47.2 mmol, 1.0 eq) was dissolved in MeOH (200 mL). The mixture was purified by prep-SFC(column: DAICEL CHIRALCEL OJ(250 mm×30 mm, 10 um); mobile phase: [0.1% NH₃H₂O MEOH]; B %: 30%-30%, 2.6 min) to give benzyl (3R,4R)-3-(tert-butoxycarbonylamino)-4-fluoro-pyrrolidine-1-carboxylate (7 g, 43% yield) and benzyl (3S,4S)-3-(tert-butoxycarbonylamino)-4-fluoro-pyrrolidine-1-carboxylate (7 g, 43% yield).

Step 6. To a solution of benzyl (3R,4R)-3-(tert-butoxycarbonylamino)-4-fluoro-pyrrolidine-1-carboxylate (400 mg, 1.18 mmol, 1 eq) in CF₃CH₂OH (20 mL) was added Pd/C (50 mg, 10% purity) under N₂ atmosphere. The mixture was stirred under H₂ (15 Psi) at 20° C. for 1 h. The mixture was filtered and concentrated to give tert-butyl N-[(3R,4R)-4-fluoropyrrolidin-3-yl]carbamate (240 mg) as a yellow oil.

Step 7. To a solution of 3-methoxy-1-methyl-pyrazol-4-amine (2.4 g, 19 mmol, 1 eq) and 2,6-dichloro-9-methyl-purine (3.83 g, 19 mmol, 1 eq) in IPA (40 mL) was added DIEA (2.44 g, 19 mmol, 1 eq). The mixture was stirred at 85° C. for 12 h. The mixture was filtered and concentrated to give 2-chloro-N-(3-methoxy-1-methyl-pyrazol-4-yl)-9-methyl-purin-6-amine (5.1 g, 92% yield). ¹H NMR (400 MHz, CDCl₃): δ 8.01 (s, 1H), 7.74 (s, 1H), 7.44 (m, 1H), 3.97 (s, 3H), 3.82 (s, 3H), 3.79 (s, 3H)

Step 8. To a solution of 2-chloro-N-(3-methoxy-1-methyl-pyrazol-4-yl)-9-methyl-purin-6-amine (1.33 g, 5 mmol, 1 eq) and tert-butyl N-[(3R,4R)-4-fluoropyrrolidin-3-yl]carbamate (1.2 g, 6 mmol, 1.3 eq) in NMP (12 mL) was added DIEA (3 g, 23 mmol, 5 eq). The mixture was stirred at 130° C. for 12 h and purified by prep-HPLC (neutral condition; column: Waters Xbridge C18 150×50 mm×10 um; mobile phase: [water(NH₄HCO₃)-ACN]; B %: 28%-58%, 10 min) to give tert-butyl N-[(3R,4R)-4-fluoro-1-[6-[(3-methoxy-1-methyl-pyrazol-4-yl)amino]-9-methyl-purin-2-yl]pyrrolidin-3-yl]carbamate (1.51 g, 72% yield). ¹H NMR (400 MHz, CD₃OD): δ 8.00-7.94 (m, 1H), 7.73-7.66 (m, 1H), 5.23-5.04 (m, 1H), 4.28 (m, 1H), 3.98 (s, 3H), 3.95-3.89 (m, 2H), 3.86 (m, 1H), 3.78-3.76 (m, 3H), 3.71-3.67 (m, 3H), 3.33 (m, 3H), 1.48 (s, 9H)

Step 9. To a solution of tert-butyl N-[(3R,4R)-4-fluoro-1-[6-[(3-methoxy-1-methyl-pyrazol-4-yl)amino]-9-methyl-purin-2-yl]pyrrolidin-3-yl]carbamate (60 mg, 130 μmol, 1 eq) in DCM (2 mL) was added TFA (1 mL). The mixture was stirred at 20° C. for 1 h and concentrated to give the desired compound 2-[(3R,4R)-3-amino-4-fluoro-pyrrolidin-1-yl]-N-(3-methoxy-1-methyl-pyrazol-4-yl)-9-methyl-purin-6-amine Compound Q (61 mg, TFA) as a yellow oil.

Step 10. To a solution of 2-((3R,4R)-3-amino-4-fluoropyrrolidin-1-yl)-N-(3-methoxy-1-methyl-1H-pyrazol-4-yl)-9-methyl-9H-purin-6-amine (300 mg, 631 μmol, 1.0 eq, TFA salt) and 2-(((triisopropylsilyl)oxy)methyl)acrylic acid (244 mg, 946 μmol, 1.5 eq) in DMF (3 mL) was added DIEA (244 mg, 1.89 mmol, 329 μL, 3.0 eq), EDCI (181 mg, 946 μmol, 1.5 eq) and HOBt (127 mg, 946 μmol, 1.5 eq). The mixture was stirred at 20° C. for 1 h. Then KF (366 mg, 6.31 mmol, 147 μL, 10 eq) and MeOH (3 mL) was added to the mixture. The mixture was stirred at 20° C. for 11 h. The reaction mixture was filtered and concentrated under reduced pressure to give a residue. The residue was purified by prep-HPLC (column: Waters Xbridge C18 150×50 mm×10 um; mobile phase: [water (NH₄HCO₃)-ACN]; B %: 12%-42%, 10 min) to give N-((3R,4R)-4-fluoro-1-(6-((3-methoxy-1-methyl-1H-pyrazol-4-yl)amino)-9-methyl-9H-purin-2-yl)pyrrolidin-3-yl)-2-(hydroxymethyl)acrylamide (220 mg, 493 μmol, 78% yield) as a white solid.

Step 11. A mixture of compound 5 (122 mg, 404 μmol, 1.5 equiv), compound 4 (120 mg, 269 μmol, 1.0 equiv) in DMF (2 mL) and THF (2 mL) was stirred at 30° C. for 1 h. No work up, the mixture with 2-(((3R,4R)-4-fluoro-1-(6-((3-methoxy-1-methyl-1H-pyrazol-4-yl)amino)-9-methyl-9H-purin-2-yl)pyrrolidin-3-yl)carbamoyl)allyl (4-nitrophenyl) carbonate (164 mg, 268.61 μmol) was obtained as a yellow oil.

### Compound D: 8-bromo-N-((5-methyl-1H-benzo[d]imidazol-2-yl)methyl)-2-(piperazin-1-yl)pyrazolo[1,5-a][1,3,5]triazin-4-amine

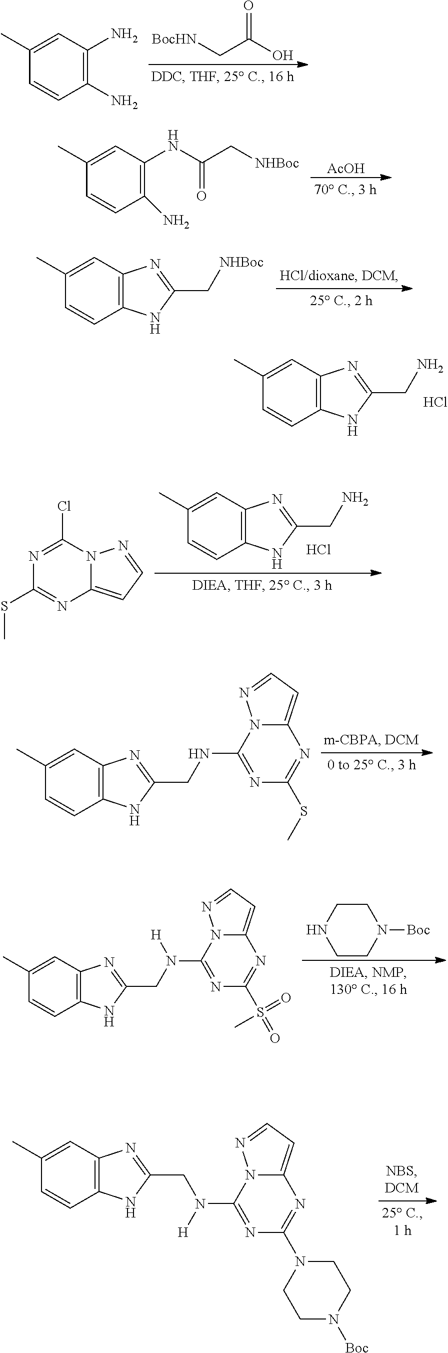

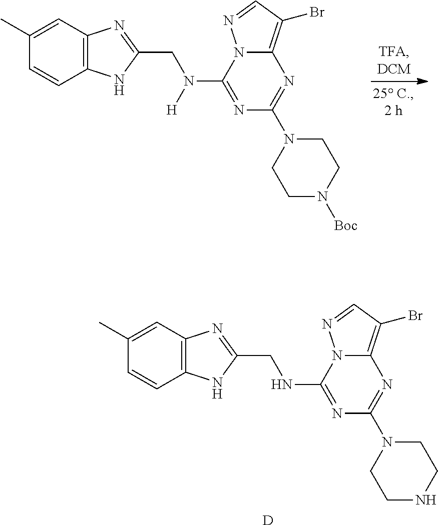

Step 1. To a solution of 4-methylbenzene-1,2-diamine (5.00 g, 40.9 mmol, 1.0 eq) and 2-(tert-butoxycarbonylamino)acetic acid (7.2 g, 40.9 mmol, 1.0 eq) in THF (100 mL) was added DCC (16.9 g, 81.9 mmol, 16.6 mL, 2.0 eq). The mixture was stirred at 25° C. for 12 h. The reaction mixture filtered and the filtrate was concentrated under reduced pressure. The residue was diluted with water (100 mL) and extracted with EtOAc (100 mL×3). The combined organic phase was washed with brine (100 mL), dried over Na₂SO₄, filtered and concentrated to obtain a residue. The residue was purified by flash silica gel chromatography (ethyl acetate/petroleum ether=3:1 to 0:1) to afford tert-butyl (2-((2-amino-5-methylphenyl)amino)-2-oxoethyl)carbamate (9.00 g, 27.7 mmol, 67% yield, 86% purity) as a black-brown solid. ¹H NMR: (400 MHz, DMSO-d₆) δ 9.07-8.95 (m, 1H), 7.07-6.93 (m, 2H), 6.49 (s, 1H), 6.40-6.31 (m, 1H), 4.79-4.63 (m, 2H), 3.75-3.67 (m, 2H), 2.15 (s, 3H), 1.39 (s, 9H).

Step 2. A solution of tert-butyl (2-((2-amino-5-methylphenyl)amino)-2-oxoethyl)carbamate (8.00 g, 28.6 mmol, 1.0 eq) in AcOH (120 mL) was stirred at 70° C. for 3 h. The mixture was concentrated then diluted with water (100 mL), then the reaction mixture was adjusted pH˜7 with saturated NaHCO₃ and extracted with EtOAc (3×100 mL). The organic layer was washed with brine (100 mL) and then dried over Na₂SO₄, filtered and concentrated under reduced pressure to afford tert-butyl ((5-methyl-1H-benzo[d]imidazol-2-yl)methyl)carbamate (8.00 g, 23.9 mmol, 83% yield, 78% purity) as a yellow solid. ¹H NMR: (400 MHz, DMSO-d₆) δ 7.43-7.30 (m, 2H), 7.29-7.18 (m, 1H), 6.99-6.89 (m, 1H), 4.35-4.28 (m, 2H), 2.38 (s, 3H), 1.45 (s, 9H).

Step 3. To a solution of tert-butyl ((5-methyl-1H-benzo[d]imidazol-2-yl)methyl)carbamate (1.20 g, 4.59 mmol, 1.0 eq) in DCM (15 mL) was added HCl/dioxane (4 M, 5 mL). The mixture was stirred at 25° C. for 2 h. The reaction mixture was concentrated under reduced pressure to afford (5-methyl-1H-benzo[d]imidazol-2-yl)methanamine (1.00 g, HCl salt) as a black-brown solid.

Step 4. To a solution of 4-chloro-2-methylsulfanyl-pyrazolo[1,5-a][1,3,5]triazine (500 mg, 2.49 mmol, 1.0 eq) in THF (10 mL) was added DIEA (966 mg, 7.5 mmol, 1.30 mL, 3.0 eq) and (5-methyl-1H-benzo[d]imidazol-2-yl)methanamine (591 mg, 3.0 mmol, 1.2 eq, HCl salt). The mixture was stirred at 25° C. for 3 h. The reaction mixture was concentrated under reduced pressure to give a residue. The crude product was purified by reversed-phase HPLC(0.1% FA condition) (column: YMC Triart C18 250×50 mm, 7 um; mobile phase: [water(FA)-ACN]; B %: 14%-44%, 20 min) to afford N-((5-methyl-1H-benzo[d]imidazol-2-yl)methyl)-2-(methylthio) pyrazolo-[1,5-a][1,3,5]triazin-4-amine (402 mg, 1.22 mmol, 49% yield, 99% purity) as a yellow solid. ¹H NMR: (400 MHz, CDCl₃) δ 8.25 (s, 1H), 7.91 (s, 1H), 7.67 (br s, 1H), 7.53-7.44 (m, 1H), 7.36 (s, 1H), 7.16-7.04 (m, 1H), 6.27 (s, 1H), 5.04 (s, 2H), 2.62 (s, 3H), 2.47 (s, 3H).

Step 5. To a solution of N-((5-methyl-1H-benzo[d]imidazol-2-yl)methyl)-2-(methylthio)pyrazolo[1,5-a][1,3,5]triazin-4-amine (200 mg, 615 μmol, 1.0 eq) in DCM (10 mL) was added m-CPBA (398 mg, 1.8 mmol, 80% purity, 3.0 eq) at 0° C. under N₂ atmosphere. Then the mixture was stirred at 25° C. for 4 h. The reaction mixture was quenched with sodium sulfite aqueous solution (10 mL) and extracted with dichloromethane (3×10 mL). The combined organic layers were washed with 0.2 N NaOH (10 mL), brine (10 mL), dried over Na₂SO₄, filtered and concentrated under reduced pressure to afford N-((5-methyl-1H-benzo[d]imidazol-2-yl)methyl)-2-(methylsulfonyl)pyrazolo[1,5-a][1,3,5]triazin-4-amine (300 mg) as a white solid.

Step 6. To a solution of N-((5-methyl-1H-benzo[d]imidazol-2-yl)methyl)-2-(methylsulfonyl)pyrazolo[1,5-a][1,3,5]triazin-4-amine (200 mg, 560 μmol, 1.0 eq) in NMP (4 mL) was added DIEA (72 mg, 560 μmol, 97 μL, 1.0 eq) and tert-butyl piperazine-1-carboxylate (104 mg, 560 μmol, 1.0 eq.). The mixture was stirred at 130° C. for 16 h. The mixture was diluted with water (20 mL), then the reaction mixture was extracted with DCM (3×10 mL). The organic layer was washed with brine (20 mL) and then dried over Na₂SO₄, filtered and concentrated under reduced pressure to give a residue. The crude product was purified by reversed-phase HPLC(column: Welch Xtimate C18 150×25 mm, 5 um; mobile phase: [water(TFA)-ACN]; B %: 20%-50%, 10 min) to afford tert-butyl 4-(4-(((5-methyl-1H-benzo [d]imidazol-2-yl)methyl)amino)pyrazolo[1,5-a][1,3,5]triazin-2-yl)piperazine-1-carboxylate (131 mg, 266 μmol, 47% yield, 94% purity) as a yellow solid. ¹H NMR: (400 MHz, CD₃OD) δ 7.87 (s, 1H), 7.68-7.60 (m, 1H), 7.58-7.52 (m, 1H), 7.48-7.37 (m, 1H), 5.95 (s, 1H), 5.24 (s, 2H), 3.62-3.55 (m, 4H), 3.28-3.19 (m, 4H), 2.54 (s, 3H), 1.44 (s, 9H).

Step 7. To a solution of tert-butyl 4-(4-(((5-methyl-1H-benzo[d]imidazol-2-yl)methyl)amino)pyrazolo[1,5-a][1,3,5]triazin-2-yl)piperazine-1-carboxylate (50 mg, 108 μmol, 1.0 eq) in DCM (5 mL) was added NBS (19 mg, 108 μmol, 1.0 eq). The mixture was stirred at 25° C. for 1 h. The reaction mixture was quenched with sodium sulfite aqueous solution (10 mL) and extracted with dichloromethane (3×10 mL). The combined organic layers were washed with brine (10 mL) and dried over Na₂SO₄, filtered and concentrated under reduced pressure to afford tert-butyl 4-(8-bromo-4-(((5-methyl-H-benzo[d]imidazol-2-yl)methyl)amino) pyrazolo[1,5-a][1,3,5]triazin-2-yl)piperazine-1-carboxylate (60.0 mg) as a white solid.

Step 8. To a solution of tert-butyl 4-(8-bromo-4-(((5-methyl-1H-benzo[d]imidazol-2-yl)methyl)amino)pyrazolo[1,5-a][1,3,5]triazin-2-yl)piperazine-1-carboxylate (50.0 mg, 92.0 μmol, 1.0 eq) in DCM (1.00 mL) was added TFA (0.5 mL). The mixture was stirred at 25° C. for 2 h. The reaction mixture was concentrated to give a residue. The residue was diluted with NaHCO₃ (2 mL). The solution was extracted with DCM (2 mL×3). The combined organic layers were washed with brine (2 mL), dried over Na₂SO₄, filtered and the filtrate was concentrated under reduced pressure. The crude product was purified by reversed-phase HPLC(column: Phenomenex luna C18 150×25 mm×10 um; mobile phase: [water(FA)-ACN]; B %: 1%-25%, 10.5 min) to afford 8-bromo-N-((5-methyl-1H-benzo[d]imidazol-2-yl)methyl)-2-(piperazin-1-yl)pyrazolo[1,5-a][1,3,5]triazin-4-amine D (2.84 mg, 5 μmol, 6% yield, 92% purity, FA salt) as a white solid. LC-MS: MS (ES⁺): RT=1.784 min, m/z=442.3 [M+H⁺], LC-MS method: AB05. ¹H NMR: (400 MHz, CD₃OD) δ 8.46 (s, 1H, HCOOH), 7.85 (s, 1H), 7.38 (d, J=8.4 Hz, 1H), 7.29 (s, 1H), 7.05 (d, J=8.4 Hz, 1H), 4.95 (s, 2H), 4.06-3.80 (m, 4H), 3.12-2.93 (m, 4H), 2.43 (s, 3H).

### Compound E: 2-((3R,4R)-3-(((5-chloro-2-((1-methyl-1H-pyrazol-4-yl)amino)-7H-pyrrolo[2,3-d]pyrimidin-4-yl)oxy)methyl)-4-methoxypyrrolidine-1-carbonyl)allyl (4-nitrophenyl) carbonate

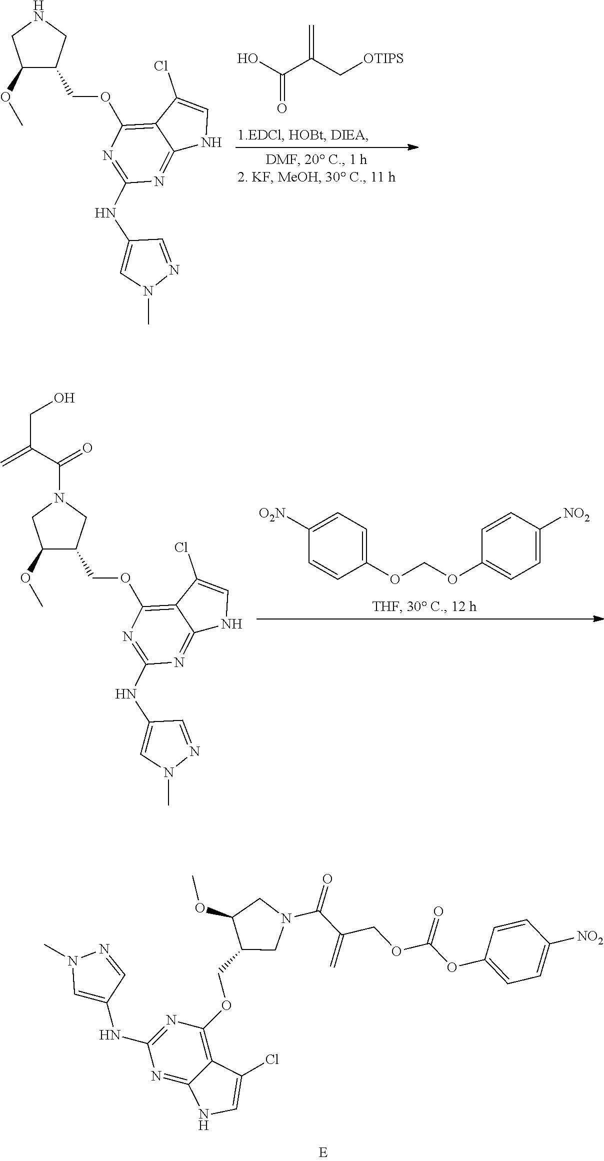

Step 1. 5-chloro-4-(((3R,4R)-4-methoxypyrrolidin-3-yl)methoxy)-N-(1-methyl-1H-pyrazol-4-yl)-7H-pyrrolo[2,3-d]pyrimidin-2-amine (J. Med. Chem., 2016, 59, 2005) and 2-(((triisopropylsilyl)oxy)methyl)acrylic acid (Bioorg. Med. Chem. Lett., 2015, 25, 5504) were prepared according to literature procedure. To a solution of 5-chloro-4-(((3R,4R)-4-methoxypyrrolidin-3-yl)methoxy)-N-(1-methyl-1H-pyrazol-4-yl)-7H-pyrrolo[2,3-d]pyrimidin-2-amine (300 mg, 609 μmol, 1.0 eq, TFA salt) and 2-(((triisopropylsilyl)oxy)methyl)acrylic acid (236 mg, 914 μmol, 1.5 eq) in DMF (3 mL) was added DIEA (394 mg, 3.05 mmol, 531 μL, 5.0 eq), HOBt (164 mg, 1.22 mmol, 2.0 eq) and EDCI (233 mg, 1.22 mmol, 2.0 eq). The mixture was stirred at 20° C. for 1 h. Then KF (354 mg, 6.10 mmol, 142 μL, 10.0 eq) and MeOH (4 mL) was added to the mixture. The mixture was stirred at 30° C. for 11 h. The reaction mixture was concentrated under reduced pressure to give a residue. The residue was purified by prep-HPLC (column: Waters Xbridge C18 150×50 mm, 10 um; mobile phase: [water(NH₄HCO₃)-ACN]; B %: 16%-46%, 10 min) to give compound 3 (170 mg, 368 μmol, 60% yield) was obtained as a white solid.

Step 2. To a solution of bis(4-nitrophenyl) carbonate (148 mg, 487 μmol, 1.5 eq) in THF (2 mL) was added 1-((3R,4R)-3-(((5-chloro-2-((1-methyl-1H-pyrazol-4-yl)amino)-7H-pyrrolo[2,3-d]pyrimidin-4-yl)oxy)methyl)-4-methoxypyrrolidin-1-yl)-2-(hydroxymethyl)prop-2-en-1-one (150 mg, 324 μmol, 1.0 eq) and stirred at 30° C. for 12 h. The mixture of E was used for next step without further purification as a yellow solution.

### Compound F: 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-2-(cyanomethyl)piperazine-1-carbonyl)allyl (4-nitrophenyl) carbonate

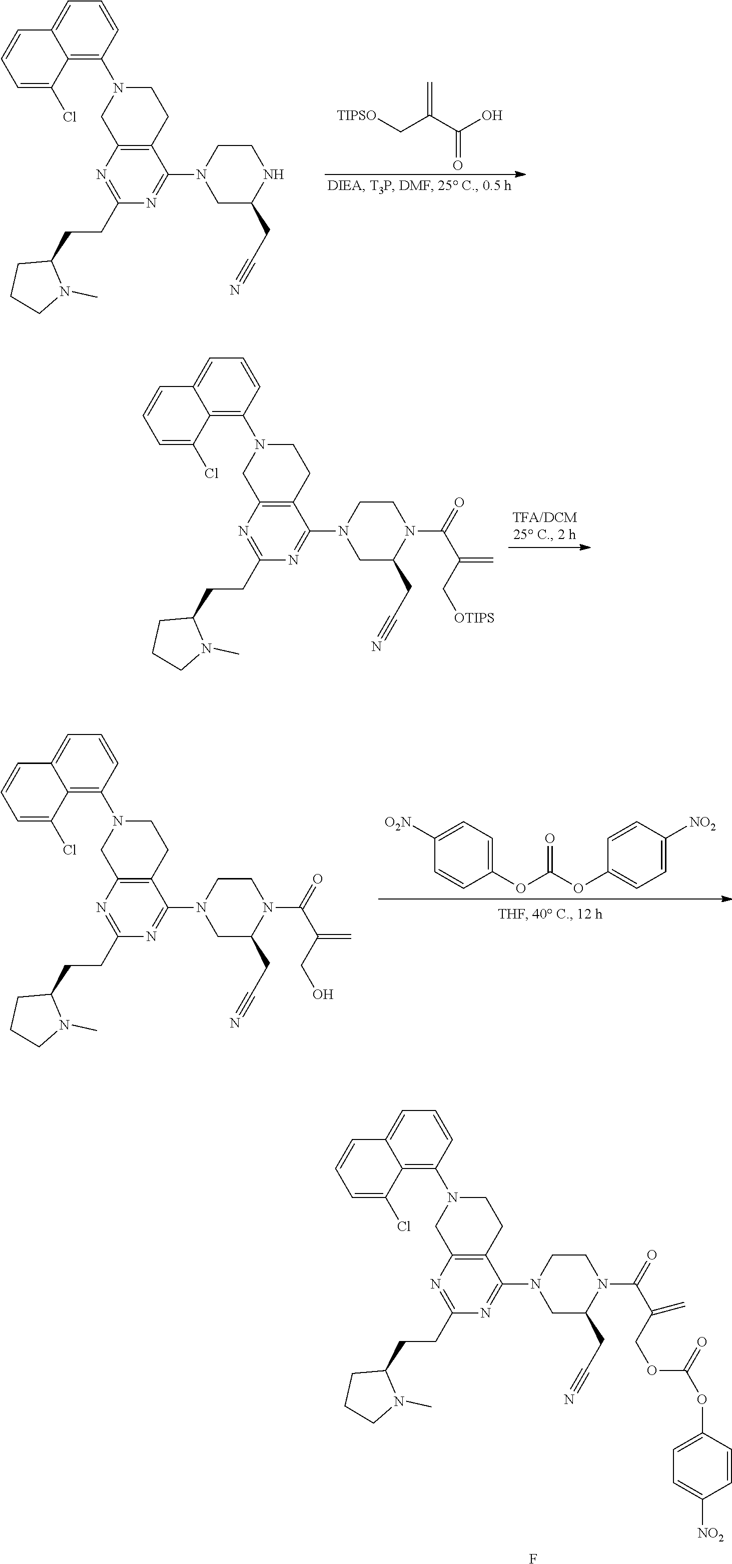

Step 1. 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (described in US2019/144444) and 2-(((triisopropylsilyl) oxy)methyl)acrylic acid (Bioorg. Med. Chem. Lett., 2015, 25, 5504) were prepared according to literature accounts. To a solution of 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (2.39 g, 3.70 mmol, 1.0 eq, TFA salt), 2-(((triisopropylsilyl)oxy) methyl)acrylic acid (1.43 g, 5.55 mmol, 1.5 eq) in DMF (20 mL) was added DIEA (1.43 g, 11.10 mmol, 1.93 mL, 3.0 eq) and T₃P (2.77 g, 5.55 mmol, 3.24 mL, 1.5 eq). The mixture was stirred at 25° C. for 0.5 h. The reaction mixture was diluted with 100 mL H₂O and extracted with EtOAc (30 mL×3). The combined organic layers were dried over Na₂SO₄, filtered and concentrated under reduced pressure to give 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-(((triisopropylsilyl)oxy)methyl)acryloyl)piperazin-2-yl)acetonitrile (5.72 g) as a brown oil and used for the next step directly.

Step 2. To a solution of 2-((S)-4-(7-(8-chloronaphthalen-1-yl)-2-(((S)-1-methylpyrrolidin-2-yl)methoxy)-5,6,7,8-tetrahydropyrido [3,4-d]pyrimidin-4-yl)-1-(2-(((triisopropylsilyl)-oxy)methyl)acryloyl)piperazin-2-yl)acetonitrile (5.70 g, 7.38 mmol, 1.0 eq) in DCM (40 mL) was added TFA (5 mL). The mixture was stirred at 25° C. for 2 h. The reaction mixture was diluted with 100 mL NaHCO₃ and extracted with DCM/MeOH (10:1, 50 mL×2). The combined organic layers were dried over Na₂SO₄, filtered and concentrated under reduced pressure to give a residue. The residue was purified by column chromatography (SiO₂, Petroleum ether/Ethyl acetate=0/1 to dichloromethane:methanol=5/1) to afford 2-((S)-4-(7-(8-chloronaphthalen-1-yl)-2-(((S)-1-methylpyrrolidin-2-yl)methoxy)-5,6,7, 8-tetrahydropyrido[3,4-d]pyrimidin-4-yl)-1-(2-(hydroxymethyl)acryloyl)piperazin-2-yl)acetonitrile (2.17 g, 3.5 mmol, 47% yield) as a brown solid. ¹H NMR: (400 MHz, DMSO-d₆) δ 7.92 (d, J=7.7 Hz, 1H), 7.74 (d, J=3.2, 7.9 Hz, 1H), 7.61-7.49 (m, 2H), 7.47-7.40 (m, 1H), 7.38-7.30 (m, 1H), 5.43 (s, 1H), 5.32-4.89 (m, 2H), 4.26-4.37 (m, J=5.5, 11.0 Hz, 1H), 4.22-4.08 (m, 4H), 3.95 (d, J=13.7 Hz, 1H), 3.88-3.64 (m, 2H), 3.62-3.36 (m, 2H), 3.26-3.01 (m, 6H), 3.00-2.58 (m, 4H), 2.52 (s, 1H), 2.38 (s, 3H), 1.96 (s, 1H), 1.80-1.58 (m, 3H).

Step 3. To a solution of 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-(hydroxymethyl)acryloyl)piperazin-2-yl)acetonitrile(100 mg, 162 μmol, 1.0 eq) in THF (2 mL) was added bis(4-nitrophenyl) carbonate (98 mg, 324 μmol, 2.0 eq) and stirred at 40° C. for 12 h. The crude product 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-2-(cyanomethyl)piperazine-1-carbonyl)allyl (4-nitrophenyl) carbonate F (130 mg) was used into the next step without further purification as a yellow solution.

### Compound G: 1-(3-(2-aminoethyl)-5-chlorophenyl)-3-((2-(2,6-dioxopiperidin-3-yl)-1-oxoisoindolin-5-yl)methyl)urea

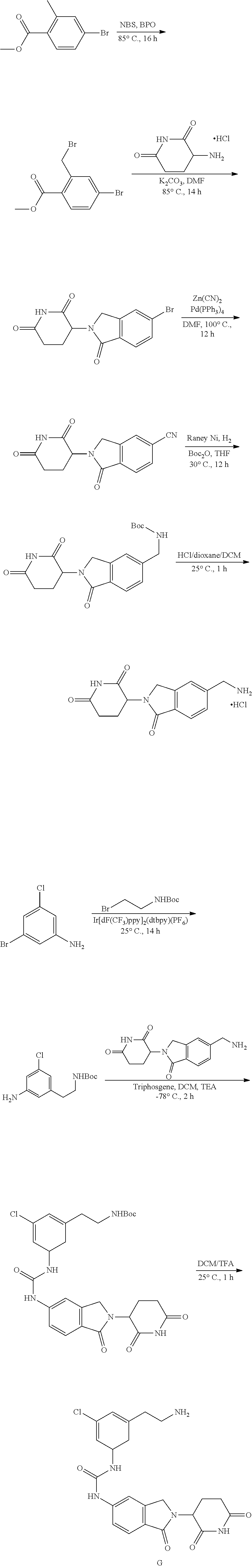

Step 1. A mixture of methyl 4-bromo-2-methyl-benzoate (40.5 g, 177 mmol, 1.0 eq), NBS (31.5 g, 177 mmol, 1.0 eq) and BPO (4.28 g, 17.7 mmol, 0.1 eq) in trifluoromethyl-benzene (400 mL) was heated to 85° C. and stirred for 16 h. To the reaction mixture was added water (500 mL) and the mixture was extracted with ethyl acetate (500 mL×3). The combined organic phase was washed with brine (500 mL), dried with anhydrous Na₂SO₄, filtered and concentrated in vacuo. The residue was purified by column chromatography on silica gel (Petroleum ether:Ethyl acetate=1:0 to 20:1) to afford methyl 4-bromo-2-(bromomethyl)benzoate (50.0 g) as a white solid. ¹H NMR (400 MHz, CDCl₃): δ 7.84 (d, 1H, J=8.4 Hz), 7.63 (d, 1H, J=2.0 Hz), 7.50 (dd, 1H, J=2.0, 8.4 Hz), 4.89 (s, 2H), 3.94 (s, 3H).

Step 2. A mixture of methyl 4-bromo-2-(bromomethyl)benzoate (4.00 g, 13.0 mmol, 1.0 eq), DIPEA (6.71 g, 51.9 mmol, 9.05 mL, 4.0 eq) and 3-aminopiperidine-2,6-dione (4.28 g, 25.9 mmol, 2.0 eq, HCl salt) in DMF (40 mL) was stirred at 50° C. for 2 h. Then the mixture was heated to 80° C. for another 12 h. The reaction mixture was poured into ice-water (100 mL) and the mixture filtered. The filter cake was washed with EtOAc (50 mL) and dried in vacuo to afford 3-(5-bromo-1-oxo-isoindolin-2-yl)piperidine-2,6-dione (4.00 g, 12.4 mmol, 95% yield) as a gray solid. ¹H NMR (400 MHz, DMSO-d₆): δ 7.88 (1H, s), 7.72-7.65 (m, 2H), 5.12-5.07 (m, 1H), 4.55-4.30 (m, 2H), 2.99-2.82 (m, 1H), 2.67-2.54 (m, 1H), 2.45-2.30 (m, 1H), 2.10-1.90 (m, 1H).

Step 3. A mixture of 3-(5-bromo-1-oxo-isoindolin-2-yl)piperidine-2,6-dione (6.30 g, 19.5 mmol, 1.0 eq), Zn(CN)₂ (1.49 g, 12.7 mmol, 804 μL, 0.65 eq) and Pd(PPh₃)₄(2.25 g, 1.95 mmol, 0.1 eq) in DMF (50 mL) was heated to 100° C. and stirred for 12 h under N₂. The reaction mixture was diluted with H₂O (30 mL). The precipitate solid was collected by filtration and triturated with ethyl acetate (80 mL). The insoluble material was collected by filtration and dried in vacuo to afford 2-(2,6-dioxo-3-piperidyl)-1-oxo-isoindoline-5-carbonitrile (5.00 g, 18.6 mmol, 95% yield) as a white solid. ¹H NMR (400 MHz, DMSO-d₆): δ 11.03 (s, 1H), 8.17 (s, 1H), 8.03-7.97 (m, 1H), 7.95-7.89 (m, 1H), 5.21-5.11 (m, 1H), 4.60-4.51 (m, 1H), 4.48-4.39 (m, 1H), 2.99-2.90 (m, 1H), 2.66-2.57 (m, 1H), 2.46-2.36 (m, 1H), 2.09-1.98 (m, 1H).

Step 4. To a mixture of 2-(2,6-dioxo-3-piperidyl)-1-oxo-isoindoline-5-carbonitrile (12.0 g, 44.6 mmol, 1.0 eq) in DMF (20 mL) and THF (20 mL) was added Boc₂O (15.6 g, 71.3 mmol, 16.4 mL, 1.6 eq) and Raney Ni (4.20 g, 49.0 mmol, 1.1 eq). The mixture was degassed and stirred at 30° C. for 12 h under H₂ (50 psi). The reaction mixture was filtered and the filtrate was concentrated in vacuo. The residue was triturated with ethyl acetate (80 mL). The insoluble material was collected by filtration and dried in vacuo. The residue was purified by prep-HPLC (column: Kromasil Eternity XT 250×80 mm, 10 um; mobile phase: [water (0.225% FA)-ACN]; B %: 25%-45%, 15 min) to afford tert-butyl N-[[2-(2,6-dioxo-3-piperidyl)-1-oxo-isoindolin-5-yl]methyl]carbamate (6.00 g, 16.1 mmol, 36% yield) as a white solid. ¹H NMR (400 MHz, DMSO-d₆): δ 10.98 (s, 1H), 7.72-7.63 (m, 1H), 7.55-7.34 (m, 3H), 5.17-5.04 (m, 1H), 4.50-4.38 (m, 1H), 4.35-4.27 (m, 1H), 4.26-5.14 (m, 2H), 2.99-2.83 (m, 1H), 2.68-2.55 (m, 1H), 2.45-2.29 (m, 1H), 2.06-1.94 (m, 1H), 1.40 (s, 9H).

Step 5. A mixture of tert-butyl N-[[2-(2,6-dioxo-3-piperidyl)-1-oxo-isoindolin-5-yl]methyl]carbamate (6.00 g, 16.1 mmol, 1.0 eq) in CH₂Cl₂ (50 mL) and 4 M HCl/dioxane (50 mL) was stirred at 25° C. for 1 h. The reaction mixture was filtered and the filtrate was concentrated in vacuo to afford 3-[5-(aminomethyl)-1-oxo-isoindolin-2-yl]piperidine-2,6-dione (4.90 g, 15.8 mmol, 98% yield, HCl salt) as a white solid. ¹H NMR (400 MHz, DMSO-d₆): δ 11.00 (s, 1H), 8.62 (s, 3H), 7.83-7.71 (m, 2H), 7.69-7.58 (m, 1H), 5.18-5.09 (m, 1H), 4.53-4.44 (m, 1H), 4.40-4.30 (m, 1H), 4.20-4.09 (m, 2H), 3.01-2.86 (m, 1H), 2.68-2.57 (m, 1H), 2.46-2.35 (m, 1H), 2.08-1.95 (m, 1H), 2.08-1.95 (m, 1H).

Step 6. A mixture of 3-bromo-5-chloro-aniline (2.0 g, 9.7 mmol, 1.0 eq), tert-butyl N-(2-bromoethyl)carbamate (2.8 g, 12.6 mmol, 1.3 eq), Ir[dF(CF₃)ppy]₂(dtbpy)(PF₆) (10.8 g, 9.9 mmol, 1.0 eq), NiCl₂.dtbbpy (3.8 g, 9.7 mmol, 1.0 eq) and TTMSS (2.4 g, 9.7 mmol, 3.0 mL, 1.0 eq), Na₂CO₃ (1.1 g, 9.7 mmol, 1.0 eq) in DME (96 mL) was degassed and purged with N₂ for 3 times, and was stirred and irradiated with a 10 W blue LED lamp (3 cm away), with cooling water to keep the reaction temperature at 25° C. for 14 h. The mixture was filtered and concentrated to give the residue. The residue was purified by the prep-HPLC (column: Phenomenex C18 250×50 mm×10 um; mobile phase: [water(NH₄HCO₃)-ACN]; B %: 29%-59%, 8 min) to give the tert-butyl N-[2-(3-amino-5-chloro-phenyl)ethyl]carbamate (600 mg, 2.2 mmol, 22% yield) as a colorless oil. ¹H NMR: (400 MHz, CDCl3) δ=6.59-6.53 (m, 2H), 6.39 (s, 1H), 3.36-3.30 (m, 2H), 2.69-2.64 (m, 2H), 1.44 (s, 9H).

Step 7. To a solution of triphosgene (0.2 g, 640 μmol, 0.5 eq) in THF (30 mL) stirred at −78° C. under N₂ protection, then the tert-butyl N-[2-(3-amino-5-chloro-phenyl)ethyl]carbamate (200 mg, 738 μmol, 1.0 eq) and TEA (747 mg, 7.4 mmol, 1.1 mL, 10.0 eq) was added to the mixture and stirred at −78° C. for 30 min. Then the 3-[5-(aminomethyl)-1-oxo-isoindolin-2-yl]piperidine-2,6-dione (228 mg, 738 μmol, 1.0 eq, HCl) was added to the mixture at −78° C. and stirred for 30 min. Then the mixture was allowed stirred at 25° C. for 1 h under N₂ protection. The mixture was poured into H₂O (100 mL) and extracted with DCM (300 mL×3), then the mixture was washed with brine and concentrated to give the residue. The residue was purified by prep-HPLC(column: Phenomenex C18 250×50 mm×10 um; mobile phase: [water(NH4HCO3)-ACN]; B %: 27%-57%, 8 min) to give the tert-butyl N-[2-[3-chloro-5-[[2-(2,6-dioxo-3-piperidyl)-1-oxo-isoindolin-5-yl]methylcarbamoylamino]phenyl]ethyl]carbamate (200 mg, 350 μmol, 47% yield) as a white solid.

Step 8. A mixture of tert-butyl N-[2-[3-chloro-5-[[2-(2,6-dioxo-3-piperidyl)-1-oxo-isoindolin-5-yl]methylcarbamoylamino]phenyl]ethyl]carbamate (170 mg, 298 μmol, 1.0 eq), in DCM (10 mL), TFA (3.0 mL) was degassed and purged with N₂ for 3 times, and then the mixture was stirred at 25° C. for 1 h under N₂ atmosphere. The mixture was concentrated to give the residue. The residue was purified by the prep-HPLC(column: Phenomenex luna C18 150×25 mm×10 um; mobile phase: [water(HCl)-ACN]; B %: 1%-31%, 10 min) to give the 1-[3-(2-aminoethyl)-5-chloro-phenyl]-3-[[2-(2,6-dioxo-3-piperidyl)-1-oxo-isoindolin-5-yl]methyl]urea G (130 mg, 254 μmol, 85% yield, 99% purity, HCl) as white solid. ¹H NMR: (400 MHz, DMSO-d6) δ=10.98 (s, 1H), 9.30 (s, 1H), 7.92 (s, 3H), 7.69 (d, J=8.0 Hz, 1H), 7.58-7.49 (m, 2H), 7.44 (d, J=8.0 Hz, 1H), 7.21-7.11 (m, 2H), 6.88 (s, 1H), 5.23-4.97 (m, 1H), 4.52-4.22 (m, 4H), 3.06-2.86 (m, 3H), 2.84-2.77 (m, 2H), 2.66-2.55 (m, 1H), 2.45-2.31 (m, 1H), 2.03-1.94 (m, 1H).

### Compound H: 1-(3-(aminomethyl)-5-chlorophenyl)-3-((2-(2,6-dioxopiperidin-3-yl)-1-oxoisoindolin-5-yl)methyl)urea

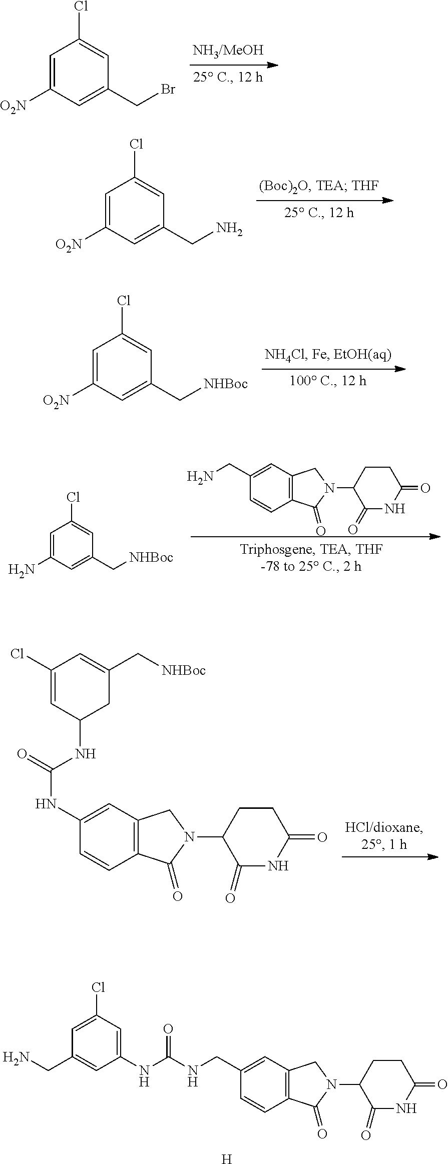

Step 1. A mixture of 1-(bromomethyl)-3-chloro-5-nitro-benzene (900 mg, 3.6 mmol, 1.0 eq) in NH₃/MeOH (5 M, 9 mL) was degassed and purged with N₂ for 3 times, and then the mixture was stirred at 25° C. for 12 h under N₂ atmosphere. The mixture was concentrated to get the residue and used for next step without further purification. The (3-chloro-5-nitro-phenyl) methanamine (670 mg, 3.6 mmol, 100% yield) was obtained as a white solid.

Step 2. To a solution of (3-chloro-5-nitro-phenyl)methanamine (670 mg, 3.4 mmol, 1.0 eq) in THF (10 mL) was added (Boc)₂O (1.6 g, 7.2 mmol, 1.7 mL, 2.0 eq) and TEA (1.1 g, 10.8 mmol, 1.5 mL, 3.0 eq). The mixture was stirred at 25° C. for 12 h. The mixture was diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by flash silica gel chromatography (ISCO®; 3 g SepaFlash® Silica Flash Column, Eluent of 0-36% Ethyl acetate/Petroleum ether gradient @ 60 mL/min) to give the tert-butyl N-[(3-chloro-5-nitro-phenyl) methyl] carbamate (800 mg, 2.8 mmol, 78% yield) as a colorless oil. ¹H NMR: (400 MHz, DMSO-d6) δ=8.15 (s, 1H), 8.07 (s, 1H), 7.77 (s, 1H), 7.65-7.58 (m, 1H), 4.25 (d, J=6.2 Hz, 2H), 1.43-1.38 (m, 9H)

Step 3. To a solution of tert-butyl N-[(3-chloro-5-nitro-phenyl)methyl]carbamate (750 mg, 2.6 mmol, 1.0 eq) in EtOH (9 mL) and H₂O (3 mL) was added NH₄Cl (1.4 g, 26.2 mmol, 10.0 eq), Fe (1.5 g, 26.2 mmol, 10.0 eq). The mixture was stirred at 100° C. for 12 h. The mixture diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by flash silica gel chromatography (ISCO®; 2 g SepaFlash® Silica Flash Column, Eluent of 0-21% Ethyl acetate/Petroleum ether gradient @ 60 mL/min) to give the tert-butyl N-[(3-amino-5-chloro-phenyl) methyl]carbamate (665 mg, 2.6 mmol, 99% yield) as a yellow solid. ¹H NMR: (400 MHz, DMSO-d6) δ=7.42-7.16 (m, 1H), 6.43 (s, 1H), 6.36 (s, 2H), 5.38 (s, 2H), 3.95 (d, J=6.2 Hz, 2H), 1.39 (s, 9H)

Step 4. To a solution of triphosgene (160 mg, 539 μmol, 0.7 eq) in THF (30 mL) stirred at −78° C. under N2 protection, then tert-butyl N-[(3-amino-5-chloro-phenyl)methyl]carbamate (200 mg, 779 μmol, 1.0 eq) and TEA (788 mg, 7.8 mmol, 1.1 mL, 10.0 eq) was added to the mixture and stirred at −78° C. for 30 min. Then the 3-[5-(aminomethyl)-1-oxo-isoindolin-2-yl] piperidine-2,6-dione (241 mg, 779 μmol, 1.0 eq, HCl) was added to the mixture at −78° C. and stirred for 30 min. Then the mixture was allowed stirred at 25° C. for 1 h under N₂ protection. The mixture was poured into H₂O (100 mL) and extracted with DCM (300 mL×3), then the mixture was washed with brine and concentrated to get the residue. The residue was purified by prep-HPLC (column: Phenomenex C18 250×50 mm×10 um; mobile phase: [water (NH₄HCO₃)-ACN]; B %: 25%-55%, 8 min) to give the tert-butyl N-[[3-chloro-5-[[2-(2,6-dioxo-3-piperidyl)-1-oxo-isoindolin-5-yl]methylcarbamoylamino]phenyl]methyl]carbamate (250 mg, 450 μmol, 58% yield) as a white solid. ¹H NMR: (400 MHz, DMSO-d6) δ=8.91 (s, 1H), 7.72-7.66 (m, 1H), 7.60-7.55 (m, 1H), 7.53-7.49 (m, 1H), 7.47-7.38 (m, 2H), 7.06 (s, 1H), 6.82-6.79 (m, 1H), 5.14-5.06 (m, 1H), 4.43-4.40 (m, 2H), 3.37 (s, 2H), 3.36 (s, 4H), 3.31 (s, 2H), 1.41-1.37 (m, 9H)

Step 5. To a solution of tert-butyl N-[[3-chloro-5-[[2-(2,6-dioxo-3-piperidyl)-1-oxo-isoindolin-5-yl]methylcarbamoylamino]phenyl]methyl]carbamate (250 mg, 450 μmol, 1.0 eq) in HCl/dioxane (20 mL). The mixture was stirred at 25° C. for 1 h. The mixture was concentrated to get the residue. The crude product was triturated with DCM (20 mL) at 25° C. to give the 1-[3-(aminomethyl)-5-chloro-phenyl]-3-[[2-(2,6-dioxo-3-piperidyl)-1-oxo-isoindolin-5-yl]methyl]urea H (220 mg, 421 μmol, 94% yield, 94% purity, HCl) as a white solid. LC-MS: MS (ES⁺): RT=1.675 min, m/z=456.3 [M+H⁺]; LC-MS Method:AB10. ¹H NMR: (400 MHz, MeOD) δ=7.76 (d, J=8.0 Hz, 1H), 7.56 (s, 1H), 7.53-7.50 (m, 1H), 7.50-7.48 (m, 2H), 7.11-7.08 (m, 1H), 5.18-5.11 (m, 1H), 4.53 (s, 2H), 4.48 (d, J=8.4 Hz, 2H), 4.05 (s, 2H), 2.95-2.74 (m, 2H), 2.55-2.42 (m, 1H), 2.20-2.13 (m, 1H).

### Compound I: 1-(3-(3-aminopropyl)-5-chlorophenyl)-3-((2-(2,6-dioxopiperidin-3-yl)-1-oxoisoindolin-5-yl)methyl)urea

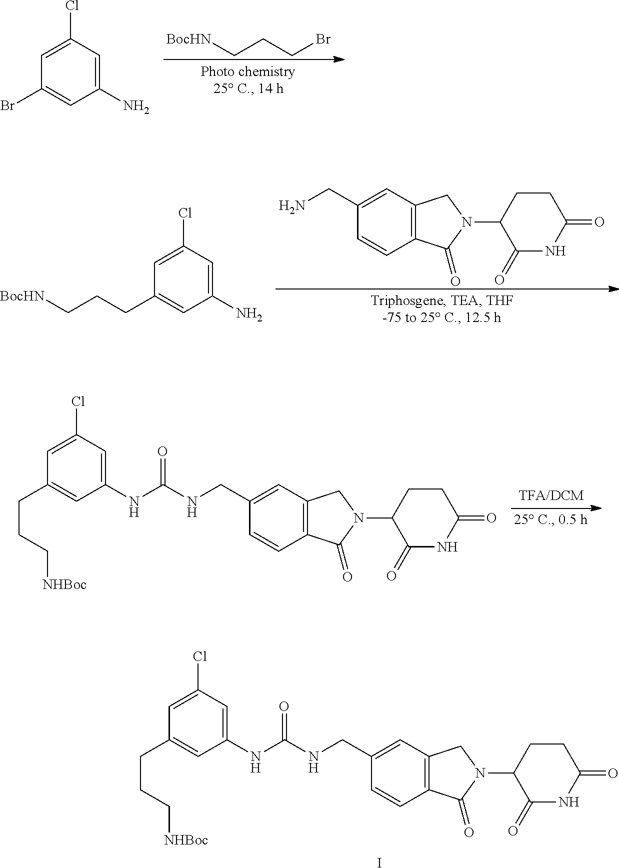

Step 1. To an 15 mL vial equipped with a stir bar was added 3-bromo-5-chloro-aniline (2.00 g, 9.69 mmol, 1.0 eq), tert-butyl N-(3-bromopropyl)carbamate (3.00 g, 12.6 mmol, 1.3 eq), Ir[dF(CF₃)ppy]₂(dtbpy)(PF₆) (109 mg, 96.9 μmol, 0.01 eq), NiCl₂.dtbbpy (193 mg, 484 μmol, 0.05 eq), TTMSS (2.41 g, 9.69 mmol, 2.99 mL, 1.0 eq), Na₂CO₃ (2.05 g, 19.4 mmol, 2.0 eq) in DME (20.0 mL). The reaction was stirred and irradiated with a 34 W blue LED lamp (7 cm away), with cooling fan to keep the reaction temperature at 25° C. for 14 h. The mixture was filtered and the filtrate was concentrated under vacuum. The residue was purified by flash silica gel chromatography (ISCO®; 40 g SepaFlash® Silica Flash Column, Eluent of 0-6% Ethyl acetate/Petroleum ether gradient @ 100 mL/min) and by prep-HPLC (FA condition; column: YMC Triart C18 250×50 mm×7 um; mobile phase: [water(FA)-ACN]; B %: 23%-53%, 20 min) to afford the product tert-butyl N-[3-(3-amino-5-chloro-phenyl)propyl]carbamate (1.36 g, 4.78 mmol, 49% yield) as a yellow oil. ¹H NMR (400 MHz, CDCl₃) (=6.58 (br d, J=13.6 Hz, 2H), 6.43 (br s, 1H), 4.55 (br d, J=1.6 Hz, 1H), 3.14 (br d, J=6.0 Hz, 3H), 2.52 (t, J=7.6 Hz, 2H), 1.77 (quin, J=7.6 Hz, 2H), 1.45 (s, 11H).

Step 2. To a solution of triphosgene (220 mg, 741 μmol, 0.85 eq) in THF (25 mL) was drop-wise added tert-butyl N-[3-(3-amino-5-chloro-phenyl)propyl]carbamate (250 mg, 878 μmol, 1.0 eq) and TEA (711 mg, 7.02 mmol, 978 μL, 8.0 eq) in THF (5 mL) at −75° C. The mixture was stirred at −75° C. for 0.5 h. 3-[5-(aminomethyl)-1-oxo-isoindolin-2-yl]piperidine-2,6-dione (299 mg, 966 μmol, 1.1 eq, HCl) was added into the reaction mixture and stirred at 25° C. for 12 h. The resultant mixture was diluted with water (60 mL), adjust the pH of the solution to 10 with NaHCO₃ (10 mL) and the aqueous phase was extracted with ethyl acetate (60 mL×2). The combined organic phase was washed with brine (60 mL×2), dried over anhydrous sodium sulfate, filtered and concentrated under vacuum to give a residue. The residue was purified by prep-HPLC (FA condition; column: Phenomenex Luna C18 200×40 mm×10 um; mobile phase: [water(FA)-ACN]; B %: 36%-66%, 10 min) to afford the desired product tert-butyl N-[3-[3-chloro-5-[[2-(2,6-dioxo-3-piperidyl)-1-oxo-isoindolin-5-yl]methylcarbamoylamino]phenyl]propyl]carbamate (226 mg, 387 μmol, 44% yield) as a white solid. ¹H NMR (400 MHz, CD₃OD-d4) δ=7.78 (d, J=8.0 Hz, 1H), 7.56 (s, 1H), 7.50 (d, J=8.0 Hz, 1H), 7.38 (s, 1H), 7.08 (s, 1H), 6.84 (s, 1H), 5.15 (dd, J=5.2, 13.2 Hz, 1H), 4.59-4.42 (m, 4H), 3.05 (br t, J=6.8 Hz, 2H), 2.96-2.85 (m, 1H), 2.82-2.73 (m, 1H), 2.60-2.53 (m, 2H), 2.52-2.43 (m, 1H), 2.22-2.12 (m, 1H), 1.83-1.70 (m, 2H), 1.43 (s, 9H).

Step 3. To a solution of tert-butyl N-[3-[3-chloro-5-[[2-(2,6-dioxo-3-piperidyl)-1-oxo-isoindolin-5-yl]methylcarbamoylamino]phenyl]propyl]carbamate (346 mg, 592 μmol, 1.0 eq) in DCM (4 mL) was added TFA (1.23 g, 10.8 mmol, 0.800 mL, 18.0 eq). The mixture was stirred at 25° C. for 0.5 h. The mixture was concentrated to give a residue. The residue was purified by prep-HPLC (FA condition; column: Phenomenex luna C18 150×25 mm×10 um; mobile phase: [water(FA)-ACN]; B %: 5%-35%, 10 min) to give the desired product 1-[3-(3-aminopropyl)-5-chloro-phenyl]-3-[[2-(2,6-dioxo-3-piperidyl)-1-oxo-isoindolin-5-yl]methyl]urea I (153 mg, 316 μmol, 53% yield) as a white solid. ¹H NMR (400 MHz, CD₃OD-d4) δ=7.77 (br d, J=8.0 Hz, 1H), 7.59-7.47 (m, 2H), 7.33-7.24 (m, 2H), 6.88 (s, 1H), 5.15 (br dd, J=5.2, 13.2 Hz, 1H), 4.56-4.45 (m, 4H), 2.96-2.85 (m, 3H), 2.83-2.74 (m, 1H), 2.66 (br t, J=7.6 Hz, 2H), 2.49 (br dd, J=4.4, 13.2 Hz, 1H), 2.22-2.13 (m, 1H), 2.00-1.89 (m, 2H) LC-MS: MS (ES⁺): RT=1.957 min, m/z=484.3 [M+H⁺]; LC-MS Method: AB05.

### Compound J: 2-(((2-(3-((2-(2,6-dioxopiperidin-3-yl)-1-oxoisoindolin-5-yl)methyl)ureido)-4-methylthiophene-3-carbonyl)oxy)methyl)acrylic acid

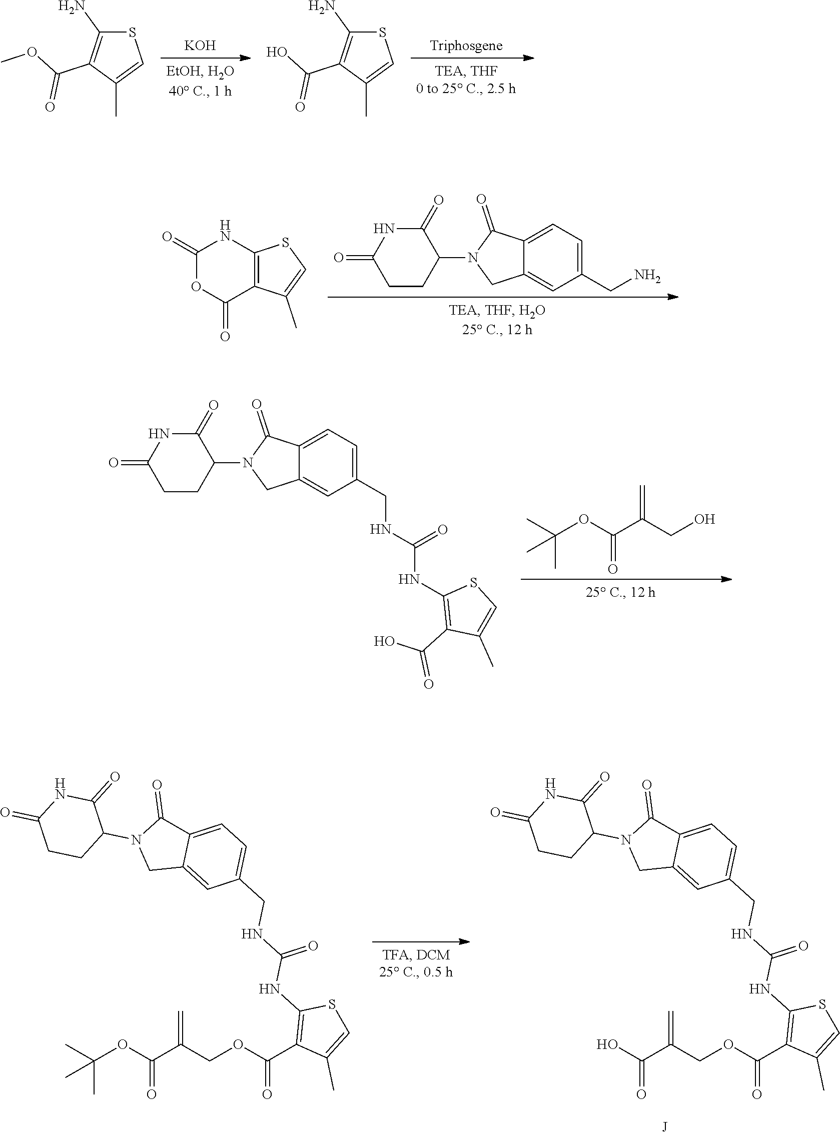

Step 1. To a solution of ethyl 2-amino-4-methyl-thiophene-3-carboxylate (2.0 g, 10.8 mmol, 1.0 eq) in H₂O (20 mL) and EtOH (20 mL) was added KOH (3.0 g, 54.0 mmol, 5.0 eq). The mixture was stirred at 40° C. for 1 h. EtOH was evaporated by vacuum distillation. The residue was diluted with water (5 mL) and washed with EtOAc (2×10 mL). The aqueous layer was cooled in an ice bath and acidized with HCl (2M) to pH=3-4. The precipitated solid was collected by filtration, washed with cold water (2×30 mL) and dried over CaCl₂ in amber glass vacuum desiccator to afford the desired product 2-amino-4-methyl-thiophene-3-carboxylic acid (1.0 g, 6.4 mmol, 59% yield) as a red solid. ¹H NMR (400 MHz, DMSO-d6) δ=7.42-7.06 (m, 2H), 5.89 (d, J=1.2 Hz, 1H), 2.15 (d, J=1.2 Hz, 3H).

Step 2. To a solution of 2-amino-4-methyl-thiophene-3-carboxylic acid (200 mg, 1.3 mmol, 1.0 eq) in THF (13 mL) was added triphosgene (320 mg, 1.1 mmol, 0.85 eq) at 0° C. The mixture was stirred at 0° C. for 0.5 h. The reaction mixture was stirred at 25° C. for 2 h. NaHCO₃ (saturated aqueous solution, 8 mL) was added cautiously, and the resulting mixture was extracted with EtOAc/THF (1/1, 2×10 mL). The combined organic layer was washed with brine (10 mL), dried (MgSO₄), and the solvent was removed by vacuum distillation. The obtained crude material was suspended in Petroleum ether: Ethyl acetate (4: 1, 10 mL), stirred in a water bath at 40° C. for 10 min, then cooled, and collected by filtration to give the desired product 5-methyl-1H-thieno[2,3-d][1,3]oxazine-2,4-dione (130 mg, 710 μmol, 56% yield) as a red solid. ¹H NMR (400 MHz, CD₃OD-d4) δ=6.42 (d, J=1.2 Hz, 1H), 2.35 (d, J=1.2 Hz, 3H).

Step 3. To a solution of 5-methyl-1H-thieno[2,3-d][1,3]oxazine-2,4-dione (130 mg, 710 μmol, 1.0 eq) in THF (9.00 mL) and H₂O (9.00 mL) was added 3-[5-(aminomethyl)-1-oxo-isoindolin-2-yl]piperidine-2,6-dione (484 mg, 1.56 mmol, 2.2 eq, HCl) and TEA (143.6 mg, 1.42 mmol, 198 μL, 2.0 eq). The reaction mixture and stirred at 25° C. for 12 h. The mixture was filtered and the filtrate was concentrated under vacuum. The residue was purified by prep-HPLC (FA condition; column: Phenomenex luna C18 150×25 mm×10 um; mobile phase: [water(FA)-ACN]; B %: 20%-50%, 10 min) to give the desired product 2-[[2-(2,6-dioxo-3-piperidyl)-1-oxo-isoindolin-5-yl]methylcarbamoylamino]-4-methyl-thiophene-3-carboxylic acid (100 mg, 216 μmol, 30% yield, 98.6% purity) as a white solid. ¹H NMR (400 MHz, DMSO-d6) (=13.04-12.55 (s, 1H), 10.99 (s, 1H), 10.71-10.53 (s, 1H), 8.47 (s, 1H), 7.71 (d, J=7.6 Hz, 1H), 7.52 (s, 1H), 7.44 (d, J=8.0 Hz, 1H), 6.40 (s, 1H), 5.11 (d, J=5.2, 13.2 Hz, 1H), 4.52-4.26 (m, 4H), 2.98-2.84 (m, 1H), 2.60 (m, 1H), 2.34 (m, 1H), 2.26 (s, 3H), 2.04-1.96 (m, 1H). LC-MS: MS (ES⁺): RT=2.185 min, m/z=457.2 [M+H⁺]; LC-MS Method: AB05.

Step 4. To a solution of 2-[[2-(2,6-dioxo-3-piperidyl)-1-oxo-isoindolin-5-yl]methylcarbamoylamino]-4-methyl-thiophene-3-carboxylic acid (140 mg, 307 μmol, 1.0 eq) and tert-butyl 2-(hydroxymethyl)prop-2-enoate (146 mg, 920 μmol, 3.0 eq) in DCM (2 mL) was added DCC (127 mg, 613 μmol, 124 μL, 2.0 eq) and DIEA (79 mg, 613 μmol, 107 μL, 2.0 eq). The mixture was stirred at 25° C. for 12 h. The mixture was filtered and the filtrate was concentrated under vacuum. The residue was purified by prep-HPLC (FA condition; column: Phenomenex luna C18 150×25 mm×10 um; mobile phase: [water(FA)-ACN]; B %: 45%-75%, 10 min). Compound 2-tert-butoxycarbonylallyl 2-[[2-(2,6-dioxo-3-piperidyl)-1-oxo-isoindolin-5-yl]methylcarbamoylamino]-4-methyl-thiophene-3-carboxylate (80 mg, 134 μmol, 44% yield) was obtained as a white solid. ¹H NMR (400 MHz, DMSO-d6) (10.97 (s, 1H), 10.46 (s, 1H), 8.52-8.49 (m, 1H), 7.71-7.69 (d, J=8.0 Hz, 1H), 7.51 (m, 1H), 7.44-7.43 (d, J=8.0 Hz, 1H), 6.45 (s, 1H), 6.22 (s, 1H), 5.91 (s, 1H), 5.12-5.08 (m, 1H), 4.94 (s, 2H), 4.44-4.34 (m, 3H), 4.29 (m, 1H), 2.90 (m, 1H), 2.57 (m, 1H), 2.37 (m, 1H), 2.23 (s, 3H), 2.00 (m, 1H), 1.43 (s, 9H).

Step 5. To a solution of 2-tert-butoxycarbonylallyl 2-[[2-(2,6-dioxo-3-piperidyl)-1-oxo-isoindolin-5-yl]methylcarbamoylamino]-4-methyl-thiophene-3-carboxylate (20 mg, 34 μmol, 1.2 eq) in DCM (0.5 mL) was added TFA (770 mg, 6.8 mmol, 0.5 mL, 200 eq). The mixture was stirred at 25° C. for 0.5 h. The mixture was concentrated to give a residue. The crude product was used into the next step without further purification. Compound 2-[[2-[[2-(2,6-dioxo-3-piperidyl)-1-oxo-isoindolin-5-yl]methylcarbamoylamino]-4-methyl-thiophene-3-carbonyl]oxymethyl]prop-2-enoic acid J (22 mg, 33 μmol, 99% yield, TFA) was obtained as a yellow oil.

### Compound K: 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

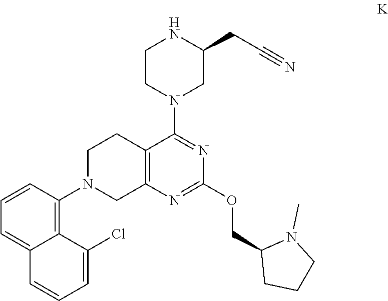 [SMILES: CN1CCC[C@H]1COc1nc2c(c(N3CCN[C@@H](CC#N)C3)n1)CCN(c1cccc3cccc(Cl)c13)C2]

Compound K can be prepared based on procedures described in Euro. J. Med. Chem., 2022, vol. 230, page 114088.

### Compound L: 2-((3-(5-((3-(3-chloro-4-methylphenyl)ureido)methyl)-1-oxoisoindolin-2-yl)-2,6-dioxopiperidin-1-yl)methyl)acrylic acid

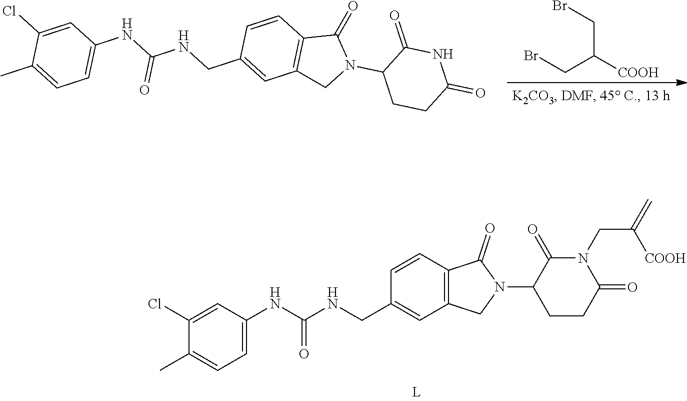 [SMILES: Cc1ccc(NC(=O)NCc2ccc3c(c2)CN(C2CCC(=O)NC2=O)C3=O)cc1Cl ; O=C(O)C(CBr)CBr ; C=C(CN1C(=O)CCC(N2Cc3cc(CNC(=O)Nc4ccc(C)c(Cl)c4)ccc3C2=O)C1=O)C(=O)O]

To a solution of 1-(3-chloro-4-methyl-phenyl)-3-[[2-(2,6-dioxo-3-piperidyl)-1-oxo-isoindolin-5-yl]methyl]urea (BRISTOL-MYERS SQUIBB CO-WO2008/27542, 2008, A2, 350 mg, 793.87 μmol, 1 eq) in DMF (7 mL) was added K₂CO₃ (329.15 mg, 2.38 mmol, 3 eq), and then it was stirred at 45° C. for 1 h. 3-bromo-2-(bromomethyl)propanoic acid (292.82 mg, 1.19 mmol, 1.5 eq) in DMF (3.5 mL) was added dropwise, and then it was stirred at 45° C. for 12 h. Half of DMF was removed by concentration, and then pH was adjusted to 6-7 by TFA. The residue was purified by prep-HPLC (column: Phenomenex luna C18 150×40 mm×15 um; mobile phase: [water(0.1% TFA)-ACN]; B %: 28%-58%, 11 min) to afford 2-[[3-[5-[[(3-chloro-4-methyl-phenyl)carbamoylamino]methyl]-1-oxo-isoindolin-2-yl]-2,6-dioxo-1-piperidyl]methyl]prop-2-enoic acid (105 mg, 200.02 μmol, 25.20% yield) as a white solid. ¹H NMR (400 MHz, DMSO): δ 12.83 (brs, 1H), 8.78 (s, 1H), 7.71-7.66 (m, 2H), 7.51 (s, 1H), 7.45-7.43 (m, 1H), 7.19-7.14 (m, 2H), 6.81 (t, 1H), 6.05 (s, 1H), 5.49 (s, 1H), 5.31 (t, 1H), 4.50-4.28 (m, 6H), 3.10-3.05 (m, 1H), 2.91-2.82 (m, 1H), 2.50-2.46 (m, 1H), 2.23 (s, 3H), 2.11-2.06 (m, 1H).

### Compound M: 6-fluoro-7-(2-fluoro-6-hydroxyphenyl)-1-(2-isopropyl-4-methylpyridin-3-yl)-4-((S)-2-methylpiperazin-1-yl)pyrido[2,3-d]pyrimidin-2(1H)-one

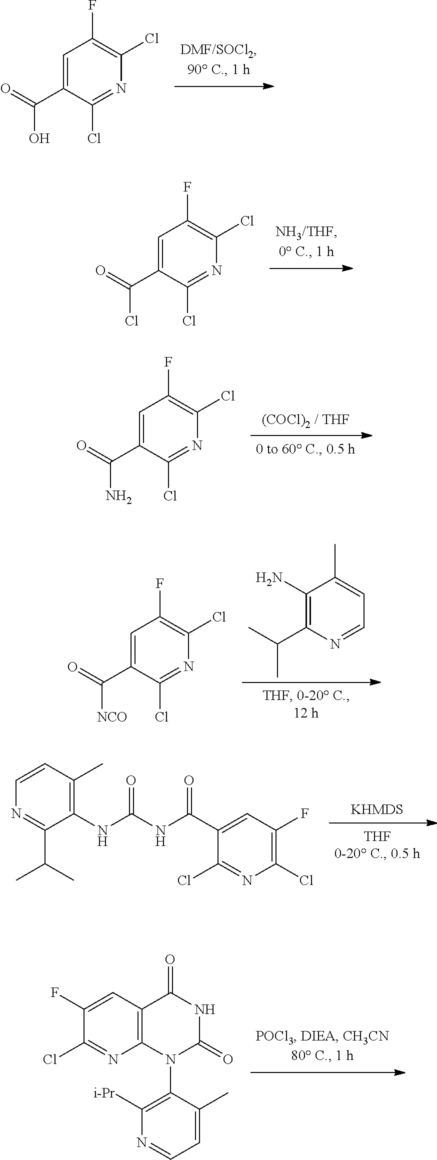

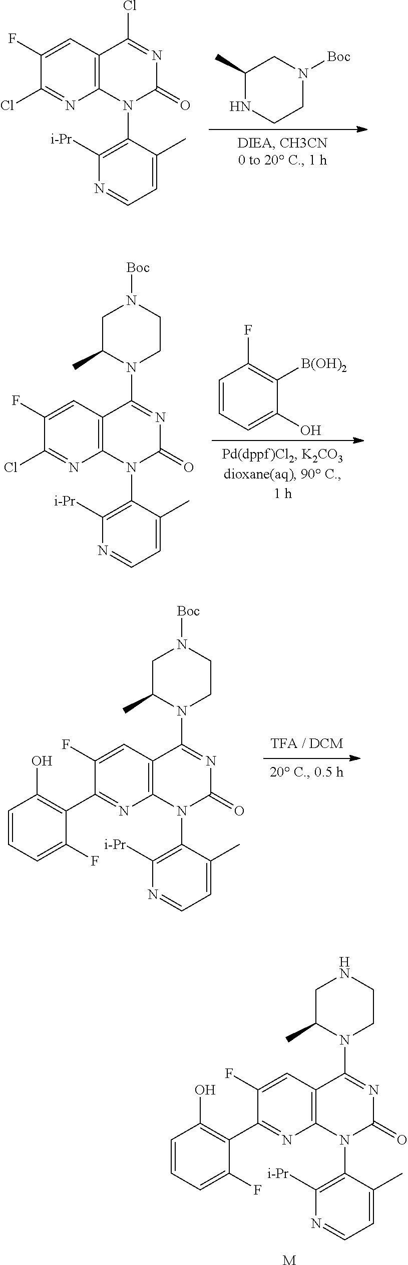

Step 1. To a solution of 2,6-dichloro-5-fluoro-pyridine-3-carboxylic acid (30.0 g, 142 mmol, 1.0 eq) in SOCl₂ (100 mL) was dropwise DMF (104 mg, 1.4 mmol, 109 μL, 0.01 eq), then it was stirred at 90° C. for 1 h. The reaction mixture was concentrated to afford a crude product. 2,6-dichloro-5-fluoro-pyridine-3-carbonyl chloride (32.6 g) was obtained as a red oil.

Step 2. To a solution of 2,6-dichloro-5-fluoro-pyridine-3-carbonyl chloride (32.6 g, 142.8 mmol, 1.0 eq) in THF (200 mL) was added NH₃ (7.0 M, 102 mL, 5.0 eq) at 0° C., then it was stirred at 0° C. for 1 h. The reaction mixture was diluted with water (100 mL) and extracted with EtOAc (200 mL×3). The combined organic phase was dried, filtered and concentrated to give 2,6-dichloro-5-fluoro-pyridine-3-carboxamide (28.0 g, 133 mmol, 93% yield) as a yellow powder. ¹H NMR (400 MHz, DMSO-d6) δ 8.24 (d, J=7.8 Hz, 1H), 8.14-7.88 (m, 2H)

Step 3. To a solution of 2,6-dichloro-5-fluoro-pyridine-3-carboxamide (9.0 g, 43 mmol, 1.0 eq) in THF (40 mL) was added oxalyl chloride (7.1 g, 55.9 mmol, 4.9 mL, 1.3 eq) at 0° C., it was stirred at 60° C. for 0.5 h. 2,6-dichloro-5-fluoro-pyridine-3-carbonyl isocyanate (10.12 g, 43.06 mmol, 100% yield) was obtained as a colorless liquid, which was used for next step directly.

Step 4. To a solution of 2,6-dichloro-5-fluoro-pyridine-3-carbonyl isocyanate (10 g, 42 mmol, 1.0 eq) in THF (100 mL) was added 2-isopropyl-4-methyl-pyridin-3-amine (6.4 g, 42.5 mmol, 1.0 eq) in THF (100 mL) at 0° C., it was stirred at 20° C. for 12 h. The reaction mixture was quenched with saturated NH₄Cl (100 mL), then extracted with EtOAc (100 mL×2). The combined organic phase was dried over anhydrous sodium sulfate, filtered and concentrated to give a residue. The crude product was purified by column chromatography (SiO₂, PE:EA=4:1). 2,6-dichloro-5-fluoro-N-[(2-isopropyl-4-methyl-3-pyridyl)carbamoyl]pyridine-3-carboxamide (8.6 g, 22.3 mmol, 52% yield) was obtained as a white solid. ¹H NMR (400 MHz, CD₃OD) δ=8.32 (s, 1H), 8.20-8.11 (m, 1H), 7.21 (s, 1H), 3.48-3.39 (m, 1H), 2.34 (s, 3H), 1.27 (d, J=3.5 Hz, 6H).

Step 5. To a solution of 2,6-dichloro-5-fluoro-N-[(2-isopropyl-4-methyl-3-pyridyl)carbamoyl]pyridine-3-carboxamide (8.6 g, 22 mmol, 1.0 eq) in THF (60 mL) was added KHMDS (1.0 M, 55 mL, 2.5 eq) at 0° C., then it was stirred at 20° C. for 0.5 h. The reaction mixture was added 50 mL NH₄Cl and extracted with EtOAc (50 mL×2). The combined organic phase was dried, filtered and concentrated to give a residue. The crude product was purified by column chromatography (SiO₂, PE:EA=4:1 to 2:1). 7-chloro-6-fluoro-1-(2-isopropyl-4-methyl-3-pyridyl)pyrido[2,3-d]pyrimidine-2,4-dione (6.8 g, 19 mmol, 87% yield) was obtained as a white solid. ¹H NMR (400 MHz, CD₃OD) δ=8.50 (d, J=5.0 Hz, 1H), 8.36 (d, J=7.2 Hz, 1H), 7.94 (d, J=7.5 Hz, 1H), 7.31 (d, J=5.0 Hz, 1H), 2.94-2.78 (m, 1H), 2.12 (s, 3H), 1.19 (d, J=6.8 Hz, 3H), 1.09 (d, J=6.7 Hz, 3H)

Step 6. To a solution of 7-chloro-6-fluoro-1-(2-isopropyl-4-methyl-3-pyridyl)pyrido[2,3-d]pyrimidine-2,4-dione (6.8 g, 19 mmol, 1.0 eq), DIEA (3.78 g, 29.2 mmol, 5.1 mL, 1.5 eq) in CH₃CN (60 mL) was added POCl₃ (3.9 g, 25.3 mmol, 2.4 mL, 1.3 eq), it was stirred at 80° C. for 1 h. The reaction mixture was concentrated to give a residue. 4,7-dichloro-6-fluoro-1-(2-isopropyl-4-methyl-3-pyridyl)pyrido[2,3-d]pyrimidin-2-one (7.2 g) was obtained as a red oil, which was used for next step directly.

Step 7. To a solution of 4,7-dichloro-6-fluoro-1-(2-isopropyl-4-methyl-3-pyridyl)pyrido[2,3-d]pyrimidin-2-one (7.2 g, 19.5 mmol, 1.0 eq) in CH₃CN (60 mL) was added DIEA (7.6 g, 58.5 mmol, 10.1 mL, 3.0 eq), tert-butyl (3S)-3-methylpiperazine-1-carboxylate (4.69 g, 23.4 mmol, 1.2 eq) at 0° C., then it was stirred at 20° C. for 1 h. The reaction mixture was diluted with saturated aqueous sodium bicarbonate solution (100 mL), extracted with EtOAc (100 mL×3). The combined organic phase was dried, filtered and concentrated to give a residue. The crude product was purified by column chromatography (SiO₂, PE:EA=3:1 to 1:1) to give tert-butyl (3S)-4-[7-chloro-6-fluoro-1-(2-isopropyl-4-methyl-3-pyridyl)-2-oxo-pyrido[2,3-d]pyrimidin-4-yl]-3-methyl-piperazine-1-carboxylate (6.1 g, 11 mmol, 58% yield) as a yellow solid. ¹H NMR (400 MHz, CD₃OD) δ=8.47 (d, J=5.0 Hz, 1H), 8.27 (d, J=8.2 Hz, 1H), 7.30 (d, J=4.8 Hz, 1H), 5.02-4.92 (m, 1H), 4.38-4.27 (m, 1H), 4.17-4.08 (m, 1H), 3.98 (d, J=13.6 Hz, 1H), 3.77 (t, J=11.2 Hz, 1H), 3.40-3.32 (m, 1H), 3.25-3.14 (m, 1H), 2.78-2.61 (m, 1H), 2.06-2.01 (m, 3H), 1.56-1.44 (m, 9H), 1.20-1.16 (m, 3H), 1.10-1.05 (m, 3H)

Step 8. To a solution of tert-butyl (3S)-4-[7-chloro-6-fluoro-1-(2-isopropyl-4-methyl-3-pyridyl)-2-oxo-pyrido[2,3-d]pyrimidin-4-yl]-3-methyl-piperazine-1-carboxylate (6.1 g, 11 mmol, 1.0 eq), (2-fluoro-6-hydroxy-phenyl)boronic acid (4.5 g, 28.7 mmol, 2.5 eq) in dioxane (60 mL), H₂O (6 mL) was added Pd(dppf)Cl₂ (672 mg, 919 μmol, 0.08 eq), K₂CO₃ (4.8 g, 34.4 mmol, 3.0 eq) at N₂ atmosphere, it was stirred at 90° C. for 1 h. The reaction system was cooled to room temperature and diluted with water (50 mL). The solution was extracted with EtOAc (50 mL×3). The combined organic phase was washed with brine (50 mL), dried, filtered and concentrated under reduced pressure. The residue was purified by column chromatography (SiO₂, PE:EA=1:1 to 0:1) to give tert-butyl (3S)-4-[6-fluoro-7-(2-fluoro-6-hydroxy-phenyl)-1-(2-isopropyl-4-methyl-3-pyridyl)-2-oxo-pyrido[2,3-d]pyrimidin-4-yl]-3-methyl-piperazine-1-carboxylate (6.6 g, 10 mmol, 95% yield) as a yellow solid. ¹H NMR (400 MHz, CD₃OD) δ=8.39 (d, J=5.0 Hz, 1H), 8.28-8.17 (m, 1H), 7.29-7.15 (m, 2H), 6.68-6.54 (m, 2H), 4.18-4.04 (m, 4H), 3.39 (s, 1H), 3.27-3.13 (m, 1H), 2.86-2.73 (m, 1H), 2.04-2.00 (m, 6H), 1.90 (d, J=5.0 Hz, 1H), 1.54-1.51 (m, 9H), 1.21-1.18 (m, 3H), 1.06-0.99 (m, 3H)

Step 9. To a solution of tert-butyl (3S)-4-[6-fluoro-7-(2-fluoro-6-hydroxy-phenyl)-1-(2-isopropyl-4-methyl-3-pyridyl)-2-oxo-pyrido[2,3-d]pyrimidin-4-yl]-3-methyl-piperazine-1-carboxylate (800 mg, 1.3 mmol, 1.0 eq) in DCM (5 mL) was added TFA (5 mL), it was stirred at 20° C. for 0.5 h. The reaction mixture was concentrated to give a TFA salt product. 6-fluoro-7-(2-fluoro-6-hydroxy-phenyl)-1-(2-isopropyl-4-methyl-3-pyridyl)-4-[(2S)-2-methylpiperazin-1-yl]pyrido[2,3-d]pyrimidin-2-one M (818 mg, 1.3 mmol, 100% yield, TFA salt) was obtained as a yellow oil.

### Compound N: (R)-3-(4-phenoxyphenyl)-1-(piperidin-3-yl)-1H-pyrazolo[3,4-d]pyrimidin-4-amine

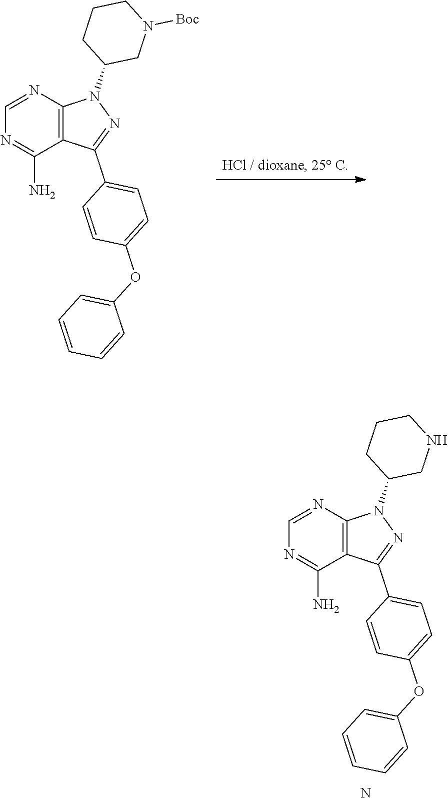

To a solution of 3-(4-phenoxyphenyl)-1-[(3R)-3-piperidyl]pyrazolo[3,4-d]pyrimidin-4-amine (WO2020/117135, 2020, A1, 50 mg, 0.13 mmol, 1 eq) in dioxane (1 mL) was added HCl/dioxane (4M, 1 mL). The mixture was stirred at 25° C. for 1 h and concentrated. The residue was purified by prep-HPLC (column: 3_Phenomenex Luna C18 75×30 mm×3 um; mobile phase: [water(0.05% HCl)-ACN]; B %: 17%-37%, 6.5 min) to give 3-(4-phenoxyphenyl)-1-[(3R)-3-piperidyl]pyrazolo[3,4-d]pyrimidin-4-amine N (38 mg, 77% yield). ¹H NMR (400 MHz, DMSO-d₆): δ 9.31-9.51 (m, 2H), 8.51 (s, 1H), 7.66-7.69 (m, 2H), 7.43-7.46 (m, 2H), 7.12-7.21 (m, 5H), 5.14-5.22 (m, 1H), 3.52-3.59 (m, 4H), 3.28-3.32 (m, 1H), 2.99-3.03 (m, 1H), 1.92-2.16 (m, 4H). LC-MS: MS (ES⁺): RT=1.947 min, m/z=387.1 [M+H⁺]; LC-MS method: AB10.

### Compound O: 2-(((3-(3-((2-(2,6-dioxopiperidin-3-yl)-1-oxoisoindolin-5-yl)methyl)ureido)phenyl)(methyl)amino)methyl)acrylic acid

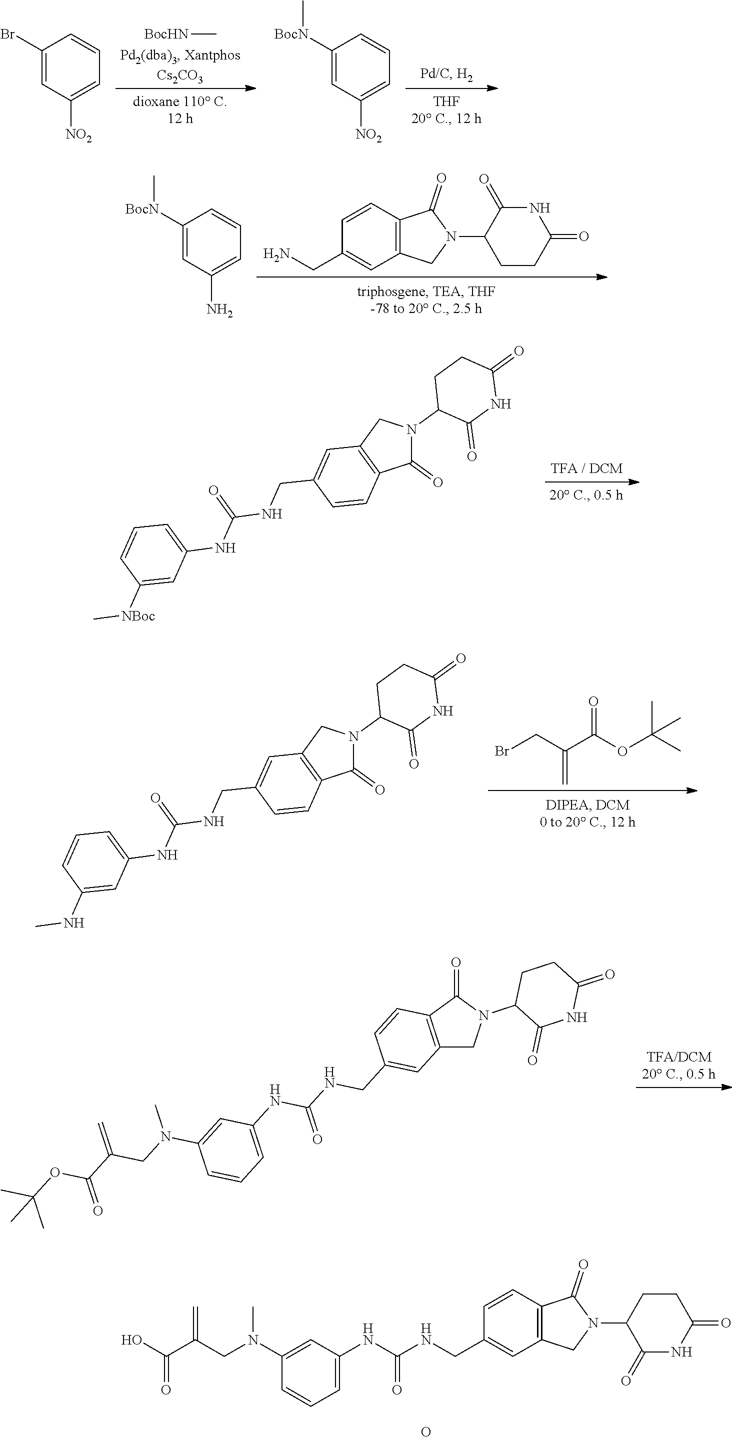

Step 1. The mixture of 1-bromo-3-nitro-benzene (3 g, 15 mmol, 1.0 eq) and tert-butyl N-methylcarbamate (2.9 g, 22 mmol, 1.5 eq) and Cs₂CO₃ (9.68 g, 29.7 mmol, 2.0 eq) and Pd₂(dba)₃ (680 mg, 743 μmol, 0.05 eq) and Xantphos (430 mg, 743 μmol, 0.05 eq) in dioxane (30 mL) was stirred at 110° C. for 12 h under N₂. The mixture was filtered and the filtrate was concentrated under reduced pressure to get the residue, which was purified by prep-HPLC (column: Welch Ultimate XB-CN 250×70×10 um; mobile phase: [Hexane-EtOH]; B %: 1%-25%, 15 min) to give tert-butyl N-methyl-N-(3-nitrophenyl)carbamate (3.5 g, 93% yield) as colorless oil. ¹H NMR (400 MHz, CDCl₃): δ 8.11-8.07 (m, 1H), 7.97-7.90 (m, 1H), 7.61-7.52 (m, 1H), 7.48-7.34 (m, 1H), 3.26 (s, 3H), 1.42 (s, 9H).

Step 2. To a solution of tert-butyl N-methyl-N-(3-nitrophenyl)carbamate (3.5 g, 13.9 mmol, 1.0 eq) in THF (20 mL) was added Pd/C (0.5 g, 10% purity) under N₂. The suspension was degassed under vacuum and purged with H₂. The mixture was stirred under H₂ (15 psi) at 20° C. for 12 h. The mixture was filtered and the filtrate was concentrated under reduced pressure to give tert-butyl N-(3-aminophenyl)-N-methyl-carbamate (3 g, 97% yield) as colorless oil. ¹H NMR (400 MHz, CDCl₃): δ 7.15-7.06 (m, 1H), 6.70-6.58 (m, 2H), 6.56-6.49 (m, 1H), 3.24 (s, 3H), 1.48 (s, 9H).

Step 3. To a solution of bis(trichloromethyl) carbonate (1.6 g, 5.3 mmol, 4.0 eq) in THF (42 mL) was added TEA (700 mg, 7.0 mmol, 964 μL, 5.1 eq) and tert-butyl N-(3-aminophenyl)-N-methyl-carbamate (300 mg, 1.35 mmol, 1.0 eq) in THF (21 mL) at −78° C., and then it was stirred for 0.5 h. 3-[5-(aminomethyl)-1-oxo-isoindolin-2-yl]piperidine-2,6-dione (460 mg, 1.48 mmol, 1.1 eq, HCl salt) was added to the mixture and then it was stirred at 20° C. for 2 h. The mixture was diluted with 10 mL H₂O and extracted with EA (10 mL×3), the combined organic layer was concentrated under reduced pressure to get the residue, which was purified by prep-HPLC (column: Waters Xbridge C18 150×50 mm×10 um; mobile phase: [water(NH₄HCO₃)-ACN]; B %: 22%-52%, 10 min) to give tert-butyl N-[3-[[2-(2,6-dioxo-3-piperidyl)-1-oxo-isoindolin-5-yl]methylcarbamoylamino]phenyl]-N-methyl-carbamate (305 mg, 584.78 μmol, 43.33% yield) as a yellow solid.

Step 4. The solution of tert-butyl N-[3-[[2-(2,6-dioxo-3-piperidyl)-1-oxo-isoindolin-5-yl]methylcarbamoylamino]phenyl]-N-methyl-carbamate (275 mg, 527 μmol, 1.0 eq) in TFA (1 mL) and DCM (3 mL) as stirred at 20° C. for 0.5 h. The solution was concentrated under reduced pressure to give 1-[[2-(2,6-dioxo-3-piperidyl)-1-oxo-isoindolin-5-yl]methyl]-3-[3-(methylamino)phenyl]urea (280 mg, TFA salt) as a brown solid.

Step 5. The solution of 1-[[2-(2,6-dioxo-3-piperidyl)-1-oxo-isoindolin-5-yl]methyl]-3-[3-(methylamino)phenyl]urea (50 mg, 93 μmol, 1.0 eq, TFA salt) in DCM (2 mL) was added DIEA (59 mg, 459.29 μmol, 0.08 mL, 4.9 eq) and tert-butyl 2-(bromomethyl)prop-2-enoate (26 mg, 118 μmol, 1.3 eq) at 0° C., then the solution was stirred at 20° C. for 12 h. The solution was concentrated under reduced pressure to get the residue, which was purified by prep-HPLC (column: Phenomenex luna C18 150×25 mm×10 um; mobile phase: [water(FA)-ACN]; B %: 36%-66%, 2 min) to give tert-butyl 2-[[3-[[2-(2,6-dioxo-3-piperidyl)-1-oxo-isoindolin-5-yl]methylcarbamoylamino]-N-methyl-anilino]methyl]prop-2-enoate (10 mg, 19% yield) as a white solid. ¹H NMR (400 MHz, DMSO-d6): δ 10.99 (s, 1H), 8.56 (s, 1H), 7.76-7.64 (m, 1H), 7.56-7.39 (m, 2H), 7.05-6.94 (m, 1H), 6.85-6.65 (m, 3H), 6.26-6.18 (m, 1H), 6.03 (s, 1H), 5.38 (s, 1H), 5.17-5.07 (m, 1H), 4.55-4.27 (m, 4H), 4.07 (s, 2H), 3.01-2.84 (m, 5H), 2.40-2.36 (m, 1H), 2.07-1.93 (m, 1H), 1.46 (s, 9H).

Step 6. The solution of tert-butyl 2-[[3-[[2-(2,6-dioxo-3-piperidyl)-1-oxo-isoindolin-5-yl]methylcarbamoylamino]-N-methyl-anilino]methyl]prop-2-enoate (85 mg, 151 μmol, 1.0 eq) in TFA (0.5 mL) and DCM (1.5 mL) was stirred at 20° C. for 0.5 h. The solution was concentrated under reduced pressure to give 2-[[3-[[2-(2,6-dioxo-3-piperidyl)-1-oxo-isoindolin-5-yl]methylcarbamoylamino]-N-methyl-anilino]methyl]prop-2-enoic acid O (76.5 mg, TFA salt) as yellow oil.

### Compound P: 2-((2-(((8-bromo-2-(piperazin-1-yl)pyrazolo[1,5-a][1,3,5]triazin-4-yl)amino)methyl)-4,5-difluoro-1H-benzo[d]imidazol-1-yl)methyl)acrylic acid

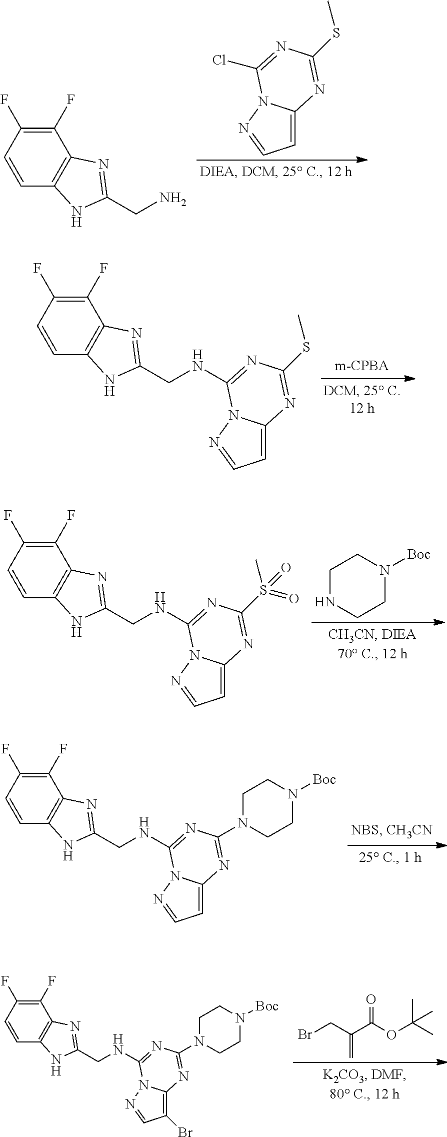

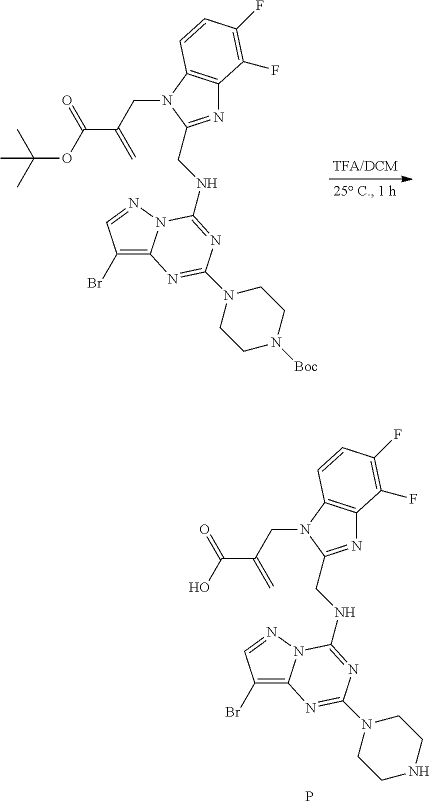

Step 1. A mixture of (4,5-difluoro-1H-benzimidazol-2-yl)methanamine (1.1 g, 3.5 mmol, 1.0 eq, TFA), 4-chloro-2-methylsulfanyl-pyrazolo[1,5-a][1,3,5]triazine (851 mg, 4.2 mmol, 1.2 eq), DIEA (2.2 g, 17.7 mmol, 3.1 mL, 5.0 eq) in DCM (10 mL), and then the mixture was stirred at 25° C. for 12 h. The reaction mixture was concentrated under reduced pressure to remove solvent. The residue was purified by column chromatography (SiO₂, Petroleum ether/Ethyl acetate=1/1 to 0/1) to give the N-[(4,5-difluoro-1H-benzimidazol-2-yl)methyl]-2-methylsulfanyl-pyrazolo[1,5-a][1,3,5]triazin-4-amine (950 mg, 2.7 mmol, 77% yield) as a light-yellow solid. ¹H NMR: (400 MHz, DMSO-d₆) δ=9.41 (t, J=5.8 Hz, 1H), 8.13 (d, J=1.6 Hz, 1H), 7.97 (d, J=1.6 Hz, 1H), 7.27-7.15 (m, 2H), 6.35 (d, J=4.0 Hz, 1H), 4.93 (d, J=6.0 Hz, 2H), 2.40 (s, 3H).

Step 2. A mixture of N-[(4,5-difluoro-1H-benzimidazol-2-yl)methyl]-2-methylsulfanyl-pyrazolo[1,5-a][1,3,5]triazin-4-amine (900 mg, 2.6 mmol, 1.0 eq) in DCM (10 mL) at 0° C., then the m-CPBA (1.8 g, 9.1 mmol, 85% purity, 3.5 eq) was added to the mixture and degassed and purged with N₂ for 3 times, and then the mixture was stirred at 25° C. for 12 h under N₂ atmosphere. The reaction mixture filtered and concentrated to get the residue together. The residue was diluted with H₂O (10 mL) and extracted with DCM (10 mL×3). The combined organic layers were washed with brine (10 mL×3), dried over Na₂SO₄, filtered and concentrated under reduced pressure to give N-[(4,5-difluoro-1H-benzimidazol-2-yl)methyl]-2-methylsulfonyl-pyrazolo[1,5-a][1,3,5]triazin-4-amine (884 mg, 2.3 mmol, 90% yield) as a yellow oil. LC-MS: MS (ES⁺): RT=0.342 min, m/z=380.0 [M+H⁺]; LC-MS Method AB05.

Step 3. A mixture of N-[(4,5-difluoro-1H-benzimidazol-2-yl)methyl]-2-methylsulfonyl-pyrazolo[1,5-a][1,3,5]triazin-4-amine (844 mg, 2.2 mmol, 1.0 eq), tert-butyl piperazine-1-carboxylate (497 mg, 2.7 mmol, 1.2 eq), DIEA (575 mg, 4.5 mmol, 775 μL, 2.0 eq) in CH₃CN (8 mL) was degassed and purged with N₂ for 3 times, and then the mixture was stirred at 70° C. for 12 h under N₂ atmosphere. This reaction mixture was concentrated to give the residue. The residue was purified by prep-HPLC (column: Phenomenex luna C18 250×50 mm×15 um; mobile phase: [water(NH₄HCO₃)-ACN]; B %: 29%-59%, 20 min) to give the tert-butyl 4-[4-[(4,5-difluoro-1H-benzimidazol-2-yl)methylamino]pyrazolo[, 5-a][1,3,5]triazin-2-yl]piperazine-1-carboxylate (630 mg, 1.3 mmol, 58% yield) as a black brown solid. ¹H NMR: (400 MHz, MeOD) δ=8.07-7.94 (m, 1H), 7.82 (d, J=2.0 Hz, 1H), 7.47-7.33 (m, 1H), 7.09-7.01 (m, 1H), 5.85 (d, J=2.0 Hz, 1H), 5.03 (d, J=6.0 Hz, 2H), 3.75 (s, 4H), 3.49 (d, J=4.5 Hz, 4H), 1.51 (s, 9H).

Step 4. A mixture of tert-butyl 4-[4-[(4,5-difluoro-1H-benzimidazol-2-yl)methylamino]pyrazolo[1,5-a][1,3,5]triazin-2-yl]piperazine-1-carboxylate (590 mg, 1.2 mmol, 1.0 eq), NBS (260 mg, 1.5 mmol, 1.2 eq) in CH₃CN (6 mL) was degassed and purged with N₂ for 3 times, and then the mixture was stirred at 25° C. for 1 h under N₂ atmosphere. The reaction mixture was filtered and concentrated under reduced pressure to give a residue. The residue was diluted with water (10 mL). The mixture was extracted with DCM (10 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=20/1 to 5/1) to give the tert-butyl 4-[8-bromo-4-[(4,5-difluoro-1H-benzimidazol-2-yl)methylamino]pyrazolo[1,5-a][1,3,5]triazin-2-yl]piperazine-1-carboxylate (630 mg, 1.1 mmol, 92% yield) as a yellow solid. LC-MS: MS (ES⁺): RT=0.499 min, m/z=565.8 [M+H⁺]; LC-MS Method AB05.

Step 5. A mixture of tert-butyl 4-[8-bromo-4-[(4,5-difluoro-1H-benzimidazol-2-yl)methylamino]pyrazolo[1,5-a][1,3,5]triazin-2-yl]piperazine-1-carboxylate (610 mg, 1.1 mmol, 1.0 eq), tert-butyl 2-(bromomethyl)prop-2-enoate (287 mg, 1.3 mmol, 1.2 eq), K₂CO₃ (299 mg, 2.2 mmol, 2.0 eq) in DMF (6 mL) was degassed and purged with N₂ for 3 times, and then the mixture was stirred at 80° C. for 12 h under N₂ atmosphere. The mixture was concentrated to give the residue. The residue was purified by prep-HPLC (column: Phenomenex C18 150×25 mm×10 um; mobile phase: [water(NH₄HCO₃)-ACN]; B %: 52%-82%, 8 min) to give the tert-butyl 4-[8-bromo-4-[[1-(2-tert-butoxycarbonylallyl)-4,5-difluoro-benzimidazol-2-yl]methylamino]pyrazolo[1,5-a] [1,3,5]triazin-2-yl]piperazine-1-carboxylate (150 mg, 213 μmol, 20% yield) as a yellow solid. ¹H NMR: (400 MHz, DMSO-d₆) δ=8.03 (s, 1H), 7.50-7.44 (m, 1H), 7.35-7.27 (m, 1H), 6.16-5.76 (m, 1H), 5.29 (d, J=15.2 Hz, 1H), 4.98-4.71 (m, 2H), 4.66-4.38 (m, 3H), 3.73-3.43 (m, 4H), 3.10 (d, J=7.6 Hz, 4H), 1.42 (s, 9H), 0.80 (s, 9H).

Step 6. A mixture of tert-butyl 4-[8-bromo-4-[[1-(2-tert-butoxycarbonylallyl)-4,5-difluoro-benzimidazol-2-yl]methylamino]pyrazolo[1,5-a][1,3,5]triazin-2-yl]piperazine-1-carboxylate (100 mg, 142 μmol, 1.0 eq) in DCM (1 mL), TFA (0.3 mL) was degassed and purged with N₂ for 3 times, and then the mixture was stirred at 25° C. for 1 h under N₂ atmosphere. The mixture was concentrated to give 2-[[2-[[(8-bromo-2-piperazin-1-yl-pyrazolo[1,5-a]1[1,3,5]triazin-4-yl)amino]methyl]-4,5-difluoro-benzimidazol-1-yl]methyl]prop-2-enoic acid P (94 mg, 142 μmol, TFA salt) as a yellow oil.

### Compound R: (R)-2-(((1-hydroxybutan-2-yl)(7-isopropyl-4-((4-(pyridin-2-yl)benzyl)amino)-7H-pyrrolo[2,3-d]pyrimidin-2-yl)amino)methyl)acrylic acid

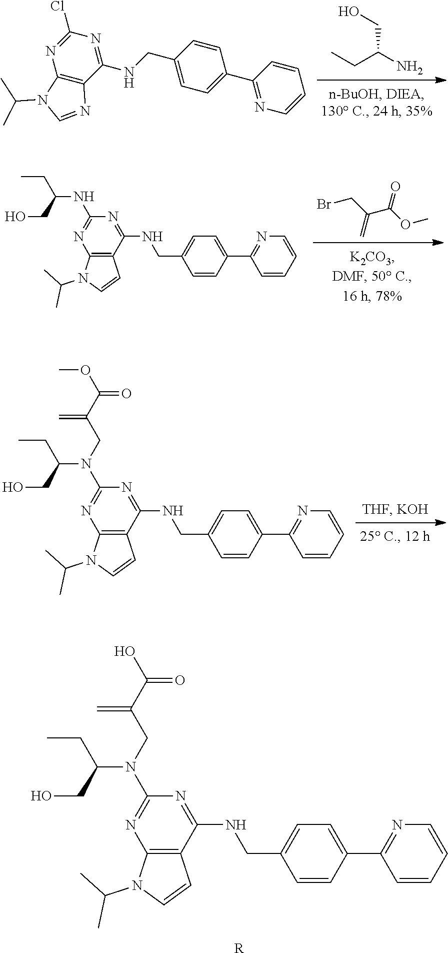

Step 1. To a solution of (2R)-2-aminobutan-1-ol (1.32 g, 14.8 mmol, 8 eq) and 2-chloro-9-isopropyl-N-[[4-(2-pyridyl)phenyl]methyl]purin-6-amine (J. Med. Chem., 2008, vol. 51, #17, p. 5229-5242, 700 mg, 1.85 mmol, 1 eq) in n-BuOH (1.5 mL) was added DIEA (1.91 g, 14.8 mmol, 8 eq). The mixture was stirred under microwave at 130° C. for 24 h. The mixture was purified by purified by prep-HPLC (column: Waters Xbridge C18 150×50 mm, 10 um; mobile phase: [water(NH₄HCO₃)-ACN]; B %: 29%-59%, 11 min) to give (2R)-2-[[9-isopropyl-6-[[4-(2-pyridyl)phenyl]methylamino]purin-2-yl]amino]butan-1-ol (280 mg, 35% yield). ¹H NMR (400 MHz, MeOD) δ 8.68-8.48 (m, 1H), 7.92-7.79 (m, 5H), 7.54-7.47 (m, 2H), 7.40-7.30 (m, 1H), 4.78-4.77 (m, 1H), 4.68-4.61 (m, 2H), 3.99-3.87 (m, 1H), 3.65-3.57 (m, 2H), 1.73-1.64 (m, 1H), 1.51-1.56 (m, 6H), 1.53-1.50 (m, 1H), 1.24-1.12 (m, 2H), 0.97-0.92 (m, 3H).

Step 2. To a solution of (2R)-2-[[9-isopropyl-6-[[4-(2-pyridyl)phenyl]methylamino]purin-2-yl]amino]butan-1-ol (130 mg, 301 μmol, 1 eq) and K₂CO₃ (125 mg, 3 eq) in DMF (1 mL) added a solution of methyl 2-(bromomethyl)prop-2-enoate (108 mg, 2 eq) in DMF (1 mL) dropwise at 50° C., the mixture was stirred at 50° C. for 16 h. The residue was purified by purified by prep-HPLC (column: Waters Xbridge 150×25 mm×5 um; mobile phase: [water(NH₄HCO₃)-ACN]; B %: 45%-75%, 10 min) to give methyl 2-[[1[(1R)-1-(hydroxymethyl)propyl]-[7-isopropyl-4-[[4-(2-pyridyl)phenyl]methylamino]pyrrolo[2,3-d]pyrimidin-2-yl]amino]methyl]prop-2-enoate (125 mg, 78% yield). ¹H NMR (400 MHz, MeOD) δ 8.58-8.57 (m, 1H), 7.92 (s, 5H), 7.80-7.68 (m, 1H), 7.44-7.31 (m, 3H), 6.31-6.18 (m, 1H), 5.63-5.57 (m, 1H), 4.70-4.55 (m, 2H), 4.05-3.94 (m, 1H), 3.94-3.84 (m, 1H), 3.76-3.72 (m, 3H), 3.61-3.57 (m, 2H), 3.36-3.34 (m, 2H), 1.66-1.60 (m, 2H), 1.55-1.51 (m, 6H), 0.96-0.90 (m, 3H).

Step 3. To a solution of methyl 2-[[[(1R)-1-(hydroxymethyl)propyl]-[7-isopropyl-4-[[4-(2-pyridyl)phenyl]methylamino]pyrrolo [2,3-d]pyrimidin-2-yl]amino]methyl]prop-2-enoate (115 mg, 1 eq) in THF (1 mL) was added KOH (0.5 M, 1.31 mL, 3 eq). The mixture was stirred at 25° C. for 12 h. The reaction mixture was adjusted pH to 6 with HCl (0.5 M, 2 mL), filtered and concentrated to give 2-[[[(1R)-1-(hydroxymethyl)propyl]-[7-isopropyl-4-[[4-(2-pyridyl)phenyl]methylamino]pyrrolo[2,3-d]pyrimidin-2-yl]amino]methyl]prop-2-enoic acid R (111 mg).

### Compound S: 2-((1-(3-chloro-4-methylphenyl)-3-((2-(2,6-dioxopiperidin-3-yl)-1-oxoisoindolin-5-yl)methyl)ureido)methyl)acrylic acid

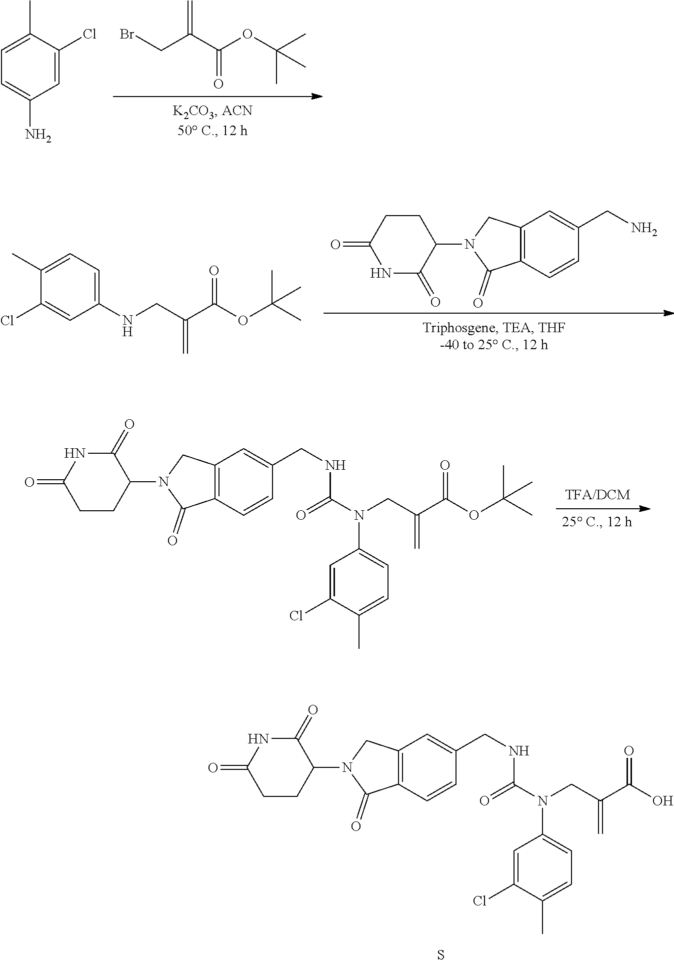

Step 1. To a solution of tert-butyl 2-(bromomethyl)prop-2-enoate (937 mg, 4.2 mmol, 1.0 eq) and 3-chloro-4-methyl-aniline (600 mg, 4.24 mmol, 1.0 eq) in MeCN (10 mL) was added K₂CO₃ (1.8 g, 12.7 mmol, 3.0 eq). Then the reaction mixture was stirred at 50° C. for 12 h. The resultant mixture was filtered and the filtrate was concentrated under vacuum. The mixture was purified by semi-preparative reverse phase HPLC (0-10% acetonitrile+0.225% formic acid in water, 10 min). to give tert-butyl 2-[(3-chloro-4-methyl-anilino)methyl]prop-2-enoate (0.45 g, 1.6 mmol, 38% yield) as a yellow oil. ¹H NMR: (400 MHz, CDCl₃) δ=7.00 (d, J=8.3 Hz, 1H), 6.63 (d, J=2.4 Hz, 1H), 6.45 (d, J=2.4, 8.2 Hz, 1H), 6.18 (d, J=0.9 Hz, 1H), 5.69 (d, J=1.4 Hz, 1H), 3.96 (s, 2H), 2.25 (s, 3H), 1.52 (s, 9H).

Step 2. To a solution of triphosgene (580 mg, 1.95 mmol, 1.6 eq) in THF (30 mL) was added drop-wise tert-butyl 2-[(3-chloro-4-methyl-anilino)methyl]prop-2-enoate (350 mg, 1.2 mmol, 1.0 eq) and TEA (1.0 g, 9.9 mmol, 1.4 mL, 8.0 eq) in THF (5 mL) at −40° C. The mixture was stirred at −40° C. for 0.5 h. 3-[5-(aminomethyl)-1-oxo-isoindolin-2-yl]piperidine-2,6-dione (373 mg, 1.4 mmol, 1.1 eq) was added into the reaction mixture and stirred at 25° C. for 12 h. The mixture was quenched with saturated aqueous solution of NaHCO₃ (3 mL) and then filtered. The filtrate was concentrated. The mixture was purified by semi-preparative reverse phase HPLC (35-55% acetonitrile+0.225% formic acid in water, 20 min) to give tert-butyl 2-[[3-chloro-N-[[2-(2,6-dioxo-3-piperidyl)-1-oxo-isoindolin-5-yl]methylcarbamoyl]-4-methyl-anilino]methyl]prop-2-enoate (240 mg, 413 μmol, 33% yield) as a white solid. ¹H NMR: (400 MHz, DMSO-d6) δ=10.98 (s, 1H), 7.65 (d, J=7.8 Hz, 1H), 7.45 (s, 1H), 7.37 (d, J=4.9, 7.9 Hz, 2H), 7.32 (d, J=2.1 Hz, 1H), 7.14 (d, J=2.1, 8.1 Hz, 1H), 6.79 (s, 1H), 6.03 (s, 1H), 5.62 (d, J=1.0 Hz, 1H), 5.11 (d, J=5.1, 13.3 Hz, 1H), 4.48-4.40 (m, 3H), 4.33-4.27 (m, 3H), 2.97-2.87 (m, 1H), 2.65-2.58 (m, 1H), 2.40 (d, J=4.5, 13.1 Hz, 1H), 2.31 (s, 3H), 2.03-1.95 (m, 1H), 1.39 (s, 9H).

Step 3. To a solution of tert-butyl 2-[[(3-chloro-4-methyl-phenyl)carbamoyl-[[2-(2,6-dioxo-3-piperidyl)-1-oxo-isoindolin-5-yl]methyl]amino]methyl]prop-2-enoate (130 mg, 224 μmol, 1.0 eq) in DCM (2 mL) was added TFA (3.1 g, 27.0 mmol, 2.00 mL, 121 eq) and the reaction mixture was stirred at 25° C. for 12 h. The resultant mixture was concentrated under vacuum. The crude product 2-[[(3-chloro-4-methyl-phenyl)carbamoyl-[[2-(2,6-dioxo-3-piperidyl)-1-oxo-isoindolin-5-yl]methyl]amino]methyl]prop-2-enoic acid S (140 mg, 219 μmol, 98% yield, TFA) was obtained as a brown oil and used into the next step without further purification.

### Compound T: 2-((1-(3-chloro-5-nitrophenyl)-3-((2-(2,6-dioxopiperidin-3-yl)-1-oxoisoindolin-5-yl)methyl)ureido)methyl)acrylic acid

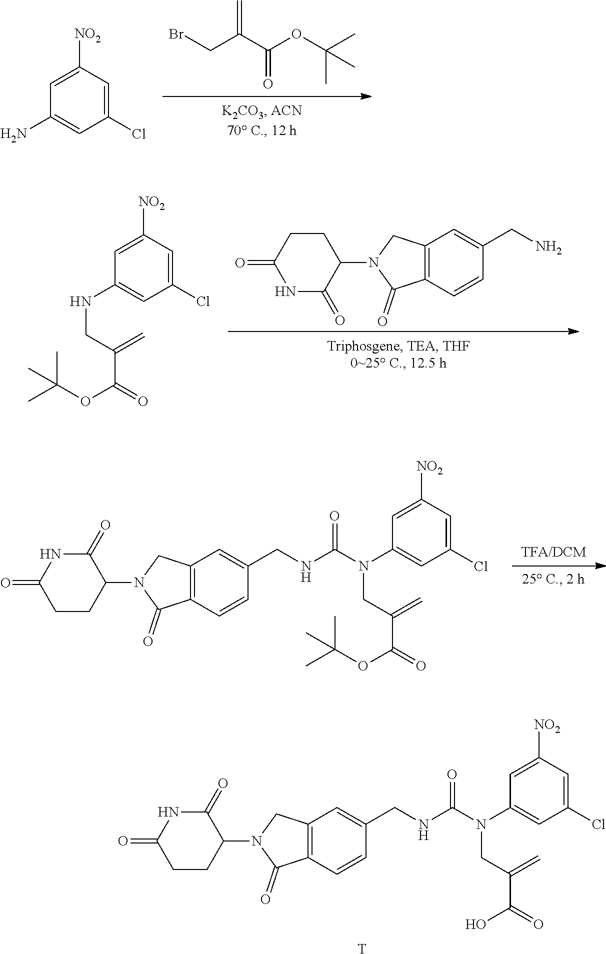

Step 1. To a solution of tert-butyl 2-(bromomethyl)prop-2-enoate (1.3 g, 5.8 mmol, 1.0 eq) and 3-chloro-5-nitro-aniline (1.0 g, 5.8 mmol, 103 μL, 1.0 eq) in MeCN (20 mL) was added K₂CO₃ (2.4 g, 17.4 mmol, 3.0 eq). Then the reaction mixture was stirred at 70° C. for 12 h. The resultant mixture was filtered and the filtrate was concentrated under vacuum. The residue was purified by flash silica gel column chromatography (2 to 10% ethyl acetate in Petroleum ether) to give the product. Compound tert-butyl 2-[(3-chloro-5-nitro-anilino)methyl]prop-2-enoate (900 mg, 2.9 mmol, 50% yield) was obtained as a red solid. ¹H NMR: (400 MHz, CDCl₃) δ=7.50 (d, J=2.0 Hz, 1H), 7.30 (d, J=2.0 Hz, 1H), 6.82 (d, J=2.0 Hz, 1H), 6.23 (s, 1H), 5.71 (s, 1H), 4.04 (s, 2H), 1.52 (s, 9H).

Step 2. To a solution of Triphosgene (60 mg, 202 μmol, 0.6 eq) in THF (5 mL) was drop-wise added tert-butyl 2-[(3-chloro-5-nitro-anilino)methyl]prop-2-enoate (100 mg, 319 μmol, 1.0 eq) and TEA (259 mg, 2.6 mmol, 356 μL, 8.0 eq) at 0° C. The mixture was stirred at 0° C. for 0.5 h. Then 3-[5-(aminomethyl)-1-oxo-isoindolin-2-yl]piperidine-2,6-dione (109 mg, 351 μmol, 1.1 eq, HCl) was added into the reaction mixture and stirred at 25° C. for 12 h. The mixture was quenched with saturated NaHCO₃ (3 mL). The resultant mixture was diluted with water (30 mL) and the aqueous phase was extracted with ethyl acetate (30 mL×2). The combined organic phase was washed with brine (20 mL×2), dried over anhydrous sodium sulfate, filtered and concentrated under vacuum to give a residue. The residue was purified by preparative-TLC (dichloromethane: methanol=10:1, Rf=0.4). Compound tert-butyl 2-[[3-chloro-N-[[2-(2,6-dioxo-3-piperidyl)-1-oxo-isoindolin-5-yl]methylcarbamoyl]-5-nitroanilino]methyl]prop-2-enoate (110 mg, 180 μmol, 56% yield) was obtained a white solid.

Step 3. To a solution of tert-butyl 2-((1-(3-chloro-5-nitrophenyl)-3-((2-(2,6-dioxopiperidin-3-yl)-1-oxoisoindolin-5-yl)methyl)ureido)methyl)acrylate (55 mg, 90 μmol, 1.0 eq) in DCM (1 mL) was added TFA (1.5 g, 13.5 mmol, 1 mL, 150 eq) and the reaction mixture was stirred at 20° C. for 2 h. The resultant mixture was concentrated under vacuum. Compound 2-[[3-chloro-N-[[2-(2,6-dioxo-3-piperidyl)-1-oxo-isoindolin-5-yl]methylcarbamoyl]-5-nitro-anilino]methyl]prop-2-enoic acid T (49 mg, 88 μmol, 98% yield) was obtained as a yellow oil and used into the next step without further purification.

### Compound U: 2-((1-(3-chloro-4-(trifluoromethyl)phenyl)-3-((2-(2,6-dioxopiperidin-3-yl)-1-oxoisoindolin-5-yl)methyl)ureido)methyl)acrylic acid

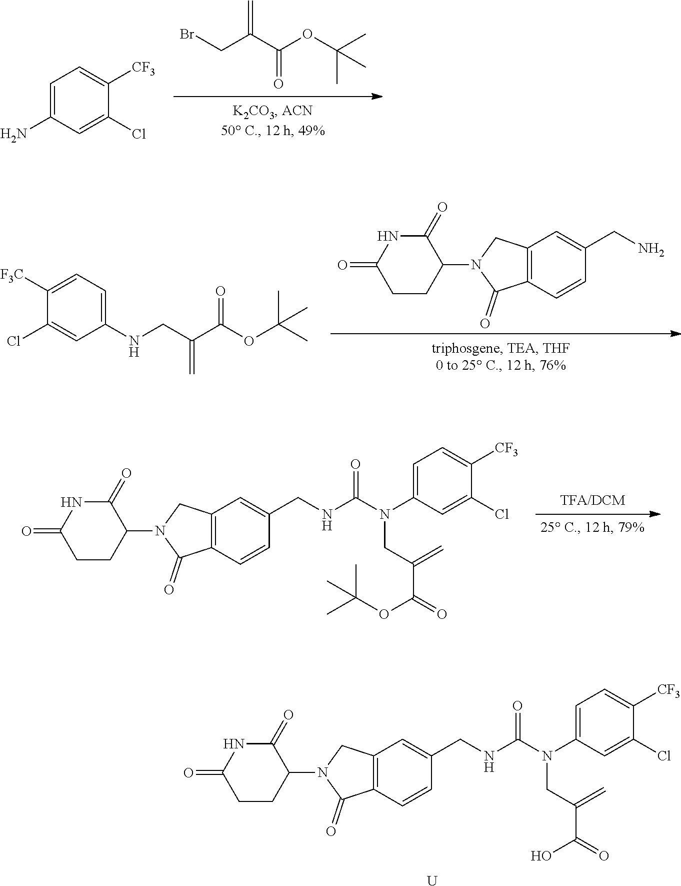

Step 1. To a solution of 3-chloro-4-(trifluoromethyl)aniline (1.0 g, 5.1 mmol, 1.0 eq) and tert-butyl 2-(bromomethyl)prop-2-enoate (1.1 g, 5.1 mmol, 1.0 eq) in MeCN (10 mL) was added K₂CO₃ (2.1 g, 15.3 mmol, 3.0 eq). The mixture was filtered, and the filtrate was concentrated under reduced pressure to afford tert-butyl 2-[(3-chloro-4-methyl-anilino)methyl]prop-2-enoate (0.7 g, 2.5 mmol, 49% yield) as a yellow solid.

Step 2. To a solution of triphosgene (0.1 g, 337 μmol, 1.1 eq) in THF (4 mL) was drop-wise added tert-butyl 2-[[4-chloro-3-(trifluoromethyl)anilino]methyl]prop-2-enoate (100 mg, 298 μmol, 1.0 eq) and TEA (241 mg, 2.4 mmol, 332 μL, 8.0 eq) in THF (4 mL) at 0° C. The mixture was stirred at 0° C. for 0.5 h. Then 3-[5-(aminomethyl)-1-oxo-isoindolin-2-yl]piperidine-2,6-dione (102 mg, 328 μmol, 1.1 eq, HCl) was added into the reaction mixture. The mixture was stirred at 25° C. for 11.5 h. The reaction mixture was quenched by addition NaHCO₃ (20 mL) at 25° C. The resultant mixture was diluted with water (15 mL) and the aqueous phase was extracted with ethyl acetate (30 mL×2). The combined organic phase was washed with brine (10 mL×2), dried over anhydrous sodium sulfate, filtered and concentrated under vacuum to give a residue to afford crude product. The crude product was purified by preparative-TLC (dichloromethane: methanol=10:1) to give 1-[3-chloro-4-(trifluoromethyl)phenyl]-1-(4,4-dimethyl-2-methylene-3-oxo-pentyl)-3-[[2-(2,6-dioxo-3-piperidyl)-1-oxo-isoindolin-5-yl]methyl]urea (140 mg, 226 μmol, 76% yield) as a yellow oil.

Step 3. To a solution of tert-butyl 2-[[3-chloro-N-[[2-(2,6-dioxo-3-piperidyl)-1-oxo-isoindolin-5-yl]methylcarbamoyl]-4-(trifluoromethyl)anilino]methyl]prop-2-enoate (70 mg, 110 μmol, 1.0 eq) in DCM (1 mL) was added TFA (1 mL). The reaction mixture was concentrated in reduced pressure to afford crude product 2-[[3-chloro-N-[[2-(2,6-dioxo-3-piperidyl)-1-oxo-isoindolin-5-yl]methylcarbamoyl]-4-(trifluoromethyl)anilino]methyl]prop-2-enoic acid U (60 mg, 87 μmol, 79% yield, TFA) as a yellow solid.

### Compound V: 2-((1-(3-chloro-4-methoxyphenyl)-3-((2-(2,6-dioxopiperidin-3-yl)-1-oxoisoindolin-5-yl)methyl)ureido)methyl)acrylic acid

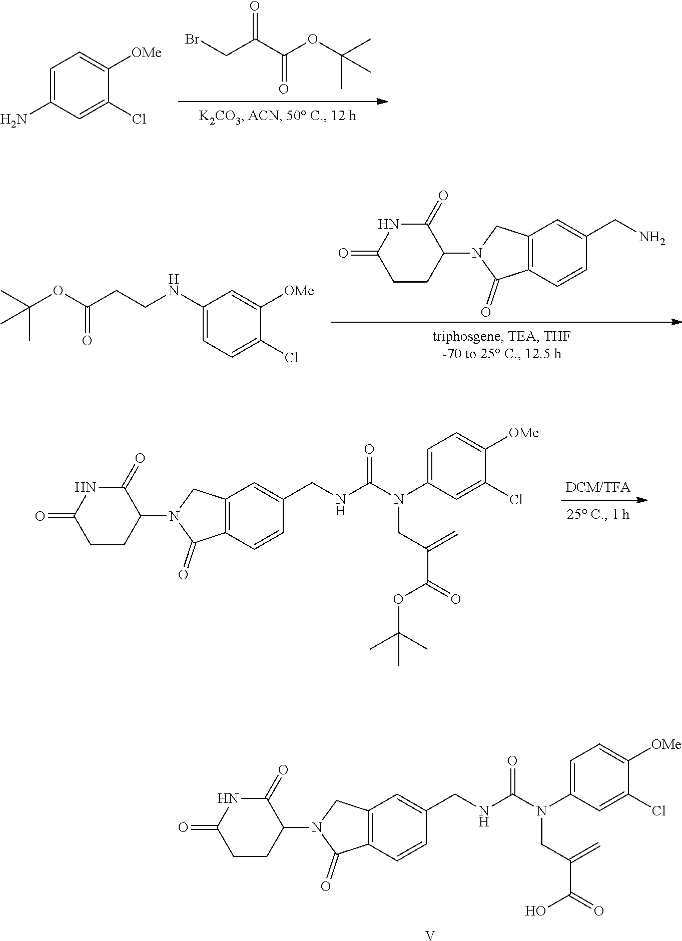

Step 1. To a solution of 3-chloro-4-methoxy-aniline (200 mg, 1.3 mmol, 1.0 eq) and tert-butyl 2-(bromomethyl)prop-2-enoate (281 mg, 1.3 mmol, 1.0 eq) in ACN (5.0 mL) was added K₂CO₃ (526 mg, 3.8 mmol, 3.0 eq). Then the reaction mixture was stirred at 50° C. for 12 h. The resultant mixture was filtered and the filtrate was concentrated under vacuum. The residue was purified by prep-TLC (SiO₂, Petroleum ether: Ethyl acetate=5:1) to afford the product tert-butyl 2-[(4-chloro-3-methoxy-anilino)methyl]prop-2-enoate (185 mg, 621 μmol, 49% yield) was obtained as a yellow oil ¹H NMR (400 MHz, CDCl₃) δ=6.80 (d, J=8.8 Hz, 1H), 6.49 (d, J=2.9, 8.8 Hz, 1H), 6.17 (d, J=1.2 Hz, 1H), 5.68 (d, J=1.6 Hz, 1H), 5.31 (s, 1H), 3.93 (s, 2H), 3.82 (s, 3H), 1.52 (s, 9H).

Step 2. To a solution of triphosgene (80 mg, 270 μmol, 0.8 eq) in THF (5.0 mL) was drop-wise added tert-butyl 2-[(4-chloro-3-methoxy-anilino)methyl]prop-2-enoate (100 mg, 336 μmol, 1.0 eq) and TEA (272 mg, 2.7 mmol, 374 μL, 8.0 eq) in THF (5.0 mL) at −70° C. The mixture was stirred at −70° C. for 0.5 h. 3-[5-(aminomethyl)-1-oxo-isoindolin-2-yl]piperidine-2,6-dione (114 mg, 369 μmol, 1.1 eq, HCl) was added into the reaction mixture and stirred at 25° C. for 12 h. The resultant mixture was diluted with saturated NaHCO₃ (30 mL) and extracted with DCM (30 mL×2). The combined organic phase was washed with brine (30 mL×2), dried over anhydrous sodium sulfate, filtered and concentrated under vacuum to give a residue. The residue was purified by prep-HPLC (FA condition; column: Phenomenex luna C18 150×25 mm×10 um; mobile phase: [water(FA)-ACN]; B %: 35%-65%, 10 min) to afford the desired product tert-butyl 2-[[3-chloro-N-[[2-(2,6-dioxo-3-piperidyl)-1-oxo-isoindolin-5-yl]methylcarbamoyl]-4-methoxy-anilino]methyl]prop-2-enoate (21.0 mg, 35.2 μmol, 10% yield) as a white solid. ¹H NMR (400 MHz, CD₃OD-d4) 8=7.76-7.71 (m, 1H), 7.49-7.46 (m, 1H), 7.43-7.39 (m, 1H), 7.30-7.26 (m, 1H), 7.19-7.14 (m, 1H), 7.13-7.09 (m, 1H), 6.12 (s, 1H), 5.63 (s, 1H), 5.15 (d, J=5.2, 13.2 Hz, 1H), 4.50-4.45 (m, 4H), 4.40 (s, 2H), 3.90 (s, 4H), 2.99-2.85 (m, 1H), 2.83-2.74 (m, 1H), 2.57-2.43 (m, 1H), 2.18 (d, J=5.2, 10.3 Hz, 1H), 1.43 (s, 9H).

Step 3. To a solution of tert-butyl 2-[[3-chloro-N-[[2-(2,6-dioxo-3-piperidyl)-1-oxo-isoindolin-5-yl]methylcarbamoyl]-4-methoxy-anilino]methyl]prop-2-enoate (21 mg, 35. mol, 1.0 eq) in DCM (1.0 mL) was added TFA (1.5 g, 13.5 mmol, 1.0 mL, 384 eq). The mixture was stirred at 25° C. for 1 h. The mixture was concentrated to give a residue. The crude product 2-[[3-chloro-N-[[2-(2,6-dioxo-3-piperidyl)-1-oxo-isoindolin-5-yl]methylcarbamoyl]-4-methoxy-anilino]methyl]prop-2-enoic acid V (20 mg, 30 μmol, 87% yield, TFA) as a yellow oil was used into the next step without further purification.

### Compound W: 2-((1-(3-chloro-5-methoxyphenyl)-3-((2-(2,6-dioxopiperidin-3-yl)-1-oxoisoindolin-5-yl)methyl)ureido)methyl)acrylic acid

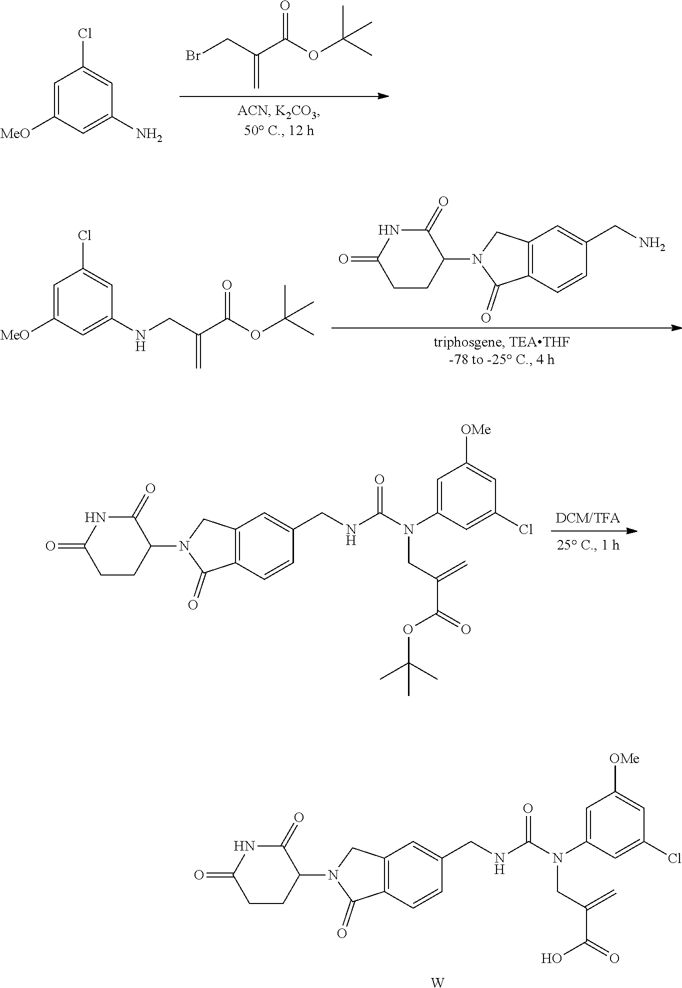

Step 1. A mixture of 3-chloro-5-methoxy-aniline (1.0 g, 6.3 mmol, 1.0 eq), tert-butyl 2-(bromomethyl)prop-2-enoate (1.4 g, 6.3 mmol, 1.0 eq), K₂CO₃ (1.8 g, 12.6 mmol, 2.0 eq) in CH₃CN (10 mL) was degassed and purged with N₂ for 3 times, and then the mixture was stirred at 50° C. for 12 h under N₂ atmosphere. The mixture was poured into H₂O (100 mL) and extracted with DCM (100 mL×2). The combined organic layers were washed with brine (100 mL×2) and dried over Na₂SO₄, then the mixture was filtered and concentrated to give the residue. The residue was purified by column chromatography (SiO₂, Petroleum ether/Ethyl acetate=10/1 to 4/1) to give the tert-butyl 2-[(3-chloro-5-methoxy-anilino)methyl]prop-2-enoate (800 mg, 2.7 mmol, 42% yield) as a colorless oil. ¹H NMR: (400 MHz, CDCl₃) δ=6.29-6.20 (m, 2H), 6.18 (s, 1H), 6.04-5.99 (m, 1H), 5.68 (s, 1H), 3.95 (s, 2H), 3.74 (s, 3H), 1.52 (s, 9H).

Step 2. To a mixture of THF (10 mL), triphosgene (70 mg, 235 μmol, 0.6 eq) stirred at −78° C. under N₂ protection was added tert-butyl 2-[(3-chloro-5-methoxy-anilino)methyl]prop-2-enoate (100 mg, 335 μmol, 1.0 eq), TEA (339 mg, 3.4 mmol, 467 μL, 10.0 eq) and the mixture was stirred for 0.5 h. Then the 3-[5-(aminomethyl)-1-oxo-isoindolin-2-yl] piperidine-2,6-dione (104 mg, 335 μmol, 1.0 eq, HCl) was added to the mixture and stirred for 1 h under N₂ protection. Then the mixture was allow stirred at 25° C. for 3 h. The mixture was poured into a saturated NH₄Cl (10 mL) and the mixture and extracted with DCM (50 mL×2). The combined organic layers were concentrated to give a residue. The residue was purified by the prep-HPLC(column: YMC Triart C18 250×50 mm×7 um; mobile phase: [water(NH4HCO3)-ACN]; B %: 39%-69%, 20 min) to give the tert-butyl 2-[[3-chloro-N-[[2-(2,6-dioxo-3-piperidyl)-1-oxo-isoindolin-5-yl]methylcarbamoyl]-5-methoxy-anilino]methyl]prop-2-enoate (50 mg, 83 μmol, 25% yield) as a white solid. ¹H NMR: (400 MHz, DMSO-d6) δ=7.65 (d, J=7.6 Hz, 1H), 7.45 (s, 1H), 7.38 (d, J=7.6 Hz, 1H), 6.96-6.87 (m, 3H), 6.82-6.75 (m, 1H), 6.03 (s, 1H), 5.63 (s, 1H), 5.18-5.01 (m, 1H), 4.52-4.25 (m, 6H), 3.75 (s, 3H), 3.01-2.82 (m, 1H), 2.63-2.57 (m, 1H), 2.46-2.34 (m, 2H), 2.05-1.94 (m, 1H), 1.39 (s, 9H).

Step 3. A mixture of tert-butyl 2-[[3-chloro-N-[[2-(2,6-dioxo-3-piperidyl)-1-oxo-isoindolin-5-yl]methylcarbamoyl]-5-methoxy-anilino]methyl]prop-2-enoate (45 mg, 75 μmol, 1.0 eq) in TFA (1 mL), DCM (3 mL) was degassed and purged with N₂ for 3 times, and then the mixture was stirred at 25° C. for 1 h under N₂ atmosphere. The mixture was concentrated to give the 2-[[3-chloro-N-[[2-(2,6-dioxo-3-piperidyl)-1-oxo-isoindolin-5-yl]methylcarbamoyl]-5-methoxy-anilino]methyl]prop-2-enoic acid (40 mg, 75 μmol) as a colorless oil.

### Compound X: 2-((1-(3-chloro-4-methylphenyl)-3-((2-(2,6-dioxopiperidin-3-yl)-1-oxoisoindolin-5-yl)methyl)ureido)methyl)acrylic acid

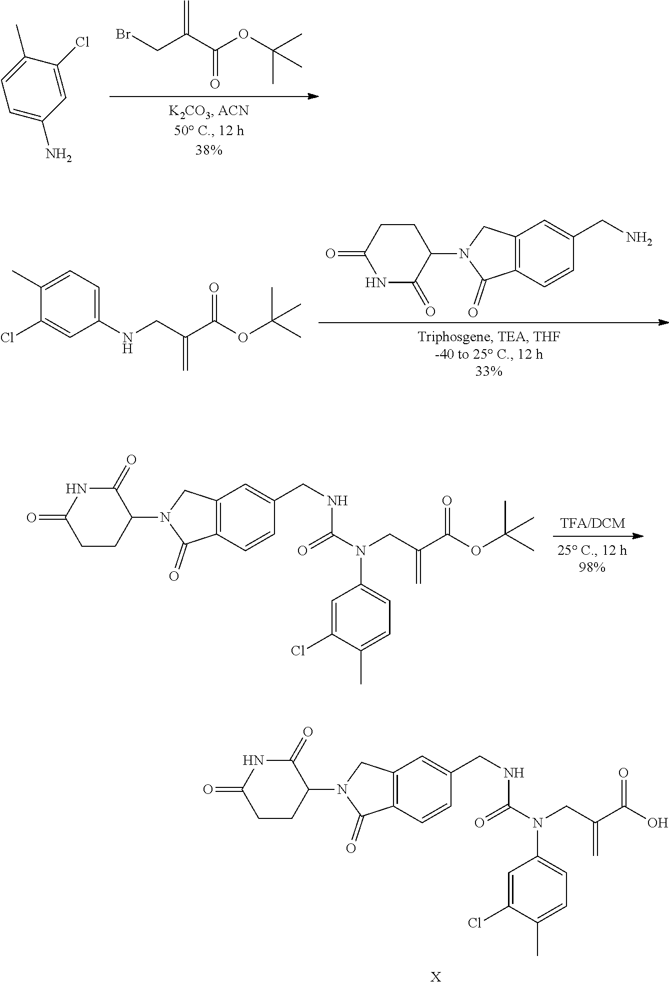

Step 1. To a solution of tert-butyl 2-(bromomethyl)prop-2-enoate (937 mg, 4.2 mmol, 1.0 eq) and 3-chloro-4-methyl-aniline (600 mg, 4.24 mmol, 1.0 eq) in MeCN (10 mL) was added K₂CO₃ (1.8 g, 12.7 mmol, 3.0 eq). Then the reaction mixture was stirred at 50° C. for 12 h. The resultant mixture was filtered and the filtrate was concentrated under vacuum. The mixture was purified by semi-preparative reverse phase HPLC (0-10% acetonitrile+0.225% formic acid in water, 10 min). to give tert-butyl 2-[(3-chloro-4-methyl-anilino)methyl]prop-2-enoate (0.45 g, 1.6 mmol, 38% yield) as a yellow oil. ¹H NMR: (400 MHz, CDCl₃) δ=7.00 (d, J=8.3 Hz, 1H), 6.63 (d, J=2.4 Hz, 1H), 6.45 (d, J=2.4, 8.2 Hz, 1H), 6.18 (d, J=0.9 Hz, 1H), 5.69 (d, J=1.4 Hz, 1H), 3.96 (s, 2H), 2.25 (s, 3H), 1.52 (s, 9H).

Step 2. To a solution of Triphosgene (580 mg, 1.95 mmol, 1.6 eq) in THF (30 mL) was added drop-wise tert-butyl 2-[(3-chloro-4-methyl-anilino)methyl]prop-2-enoate (350 mg, 1.2 mmol, 1.0 eq) and TEA (1.0 g, 9.9 mmol, 1.4 mL, 8.0 eq) in THF (5 mL) at −40° C. The mixture was stirred at −40° C. for 0.5 h. 3-[5-(aminomethyl)-1-oxo-isoindolin-2-yl]piperidine-2,6-dione (373 mg, 1.4 mmol, 1.1 eq) was added into the reaction mixture and stirred at 25° C. for 12 h. The mixture was quenched with saturated aqueous solution of NaHCO₃ (3 mL) and then filtered. The filtrate was concentrated. The mixture was purified by semi-preparative reverse phase HPLC (35-55% acetonitrile+0.225% formic acid in water, 20 min) to give tert-butyl 2-[[3-chloro-N-[[2-(2,6-dioxo-3-piperidyl)-1-oxo-isoindolin-5-yl]methylcarbamoyl]-4-methyl-anilino]methyl]prop-2-enoate (240 mg, 413 μmol, 33% yield) as a white solid. ¹H NMR: (400 MHz, DMSO-d6) δ=10.98 (s, 1H), 7.65 (d, J=7.8 Hz, 1H), 7.45 (s, 1H), 7.37 (d, J=4.9, 7.9 Hz, 2H), 7.32 (d, J=2.1 Hz, 1H), 7.14 (d, J=2.1, 8.1 Hz, 1H), 6.79 (s, 1H), 6.03 (s, 1H), 5.62 (d, J=1.0 Hz, 1H), 5.11 (d, J=5.1, 13.3 Hz, 1H), 4.48-4.40 (m, 3H), 4.33-4.27 (m, 3H), 2.97-2.87 (m, 1H), 2.65-2.58 (m, 1H), 2.40 (d, J=4.5, 13.1 Hz, 1H), 2.31 (s, 3H), 2.03-1.95 (m, 1H), 1.39 (s, 9H).

Step 3. To a solution of tert-butyl 2-[[(3-chloro-4-methyl-phenyl)carbamoyl-[[2-(2,6-dioxo-3-piperidyl)-1-oxo-isoindolin-5-yl]methyl]amino]methyl]prop-2-enoate (130 mg, 224 μmol, 1.0 eq) in DCM (2 mL) was added TFA (3.1 g, 27.0 mmol, 2.00 mL, 121 eq) and the reaction mixture was stirred at 25° C. for 12 h. The resultant mixture was concentrated under vacuum. The crude product 2-[[(3-chloro-4-methyl-phenyl)carbamoyl-[[2-(2,6-dioxo-3-piperidyl)-1-oxo-isoindolin-5-yl]methyl]amino]methyl]prop-2-enoic acid X (140 mg, 219 μmol, 98% yield, TFA) was obtained as a brown oil and used into the next step without further purification. LC-MS: MS (ES⁺): RT=0.488 min, m/z=525.1 [M+H⁺]; LC-MS Method AB05.

### Compound Y: N₁-(2-(dimethylamino)ethyl)-5-methoxy-N1-methyl-N4-(5-(1-methyl-1H-indol-3-yl)pyrimidin-2-yl)benzene-1,2,4-triamine

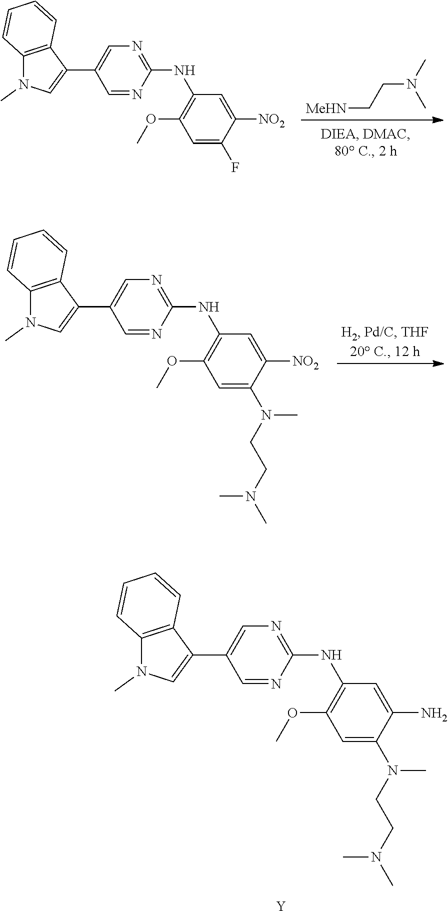

Step 1. To a solution of N-(4-fluoro-2-methoxy-5-nitro-phenyl)-4-(1-methylindol-3-yl)pyrimidin-2-amine (3 g, 7.63 mmol, 1 eq) in DMAC (10 mL) was added DIEA (2.46 g, 19 mmol, 2.5 eq) and N,N′,N′-trimethylethane-1,2-diamine (935 mg, 9.16 mmol, 1.2 eq). The mixture was stirred at 80° C. for 2 h and concentrated to give the compound N4-[2-(dimethylamino)ethyl]-2-methoxy-N4-methyl-N1-[4-(1-methylindol-3-yl)pyrimidin-2-yl]-5-nitro-benzene-1,4-diamine (2.5 g, 69% yield).

Step 2. To a solution of N4-[2-(dimethylamino)ethyl]-2-methoxy-N4-methyl-N1-[4-(1-methylindol-3-yl)pyrimidin-2-yl]-5-nitro-benzene-1,4-diamine (2.5 g, 5.26 mmol, 1 eq) in THF (15 mL) was added Pd/C (100 mg, 10% purity) under N₂. The mixture was stirred under H₂ (15 psi) at 20° C. for 12 h. The mixture was filtered and concentrated to give the compound N1-[2-(dimethylamino)ethyl]-5-methoxy-N1-methyl-N4-[4-(1-methylindol-3-yl)pyrimidin-2-yl]benzene-1,2,4-triamine Y (2.3 g, crude).

### Compound Z: 4-(3-Aminophenoxy)-N-(4-(4-methylpiperazin-1-yl)phenyl)thieno[3,2-d]pyrimidin-2-amine

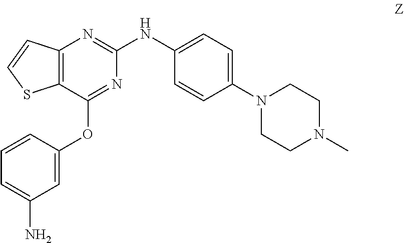 [SMILES: CN1CCN(c2ccc(Nc3nc(Oc4cccc(N)c4)c4sccc4n3)cc2)CC1]

Compound Z was prepared based on procedures described in U.S. patent application publication US2019/330229.

### Compound AA: 5-chloro-4-(((3R,4R)-4-methoxypyrrolidin-3-yl)methoxy)-N-(1-methyl-1H-pyrazol-4-yl)-7H-pyrrolo[2,3-d]pyrimidin-2-amine

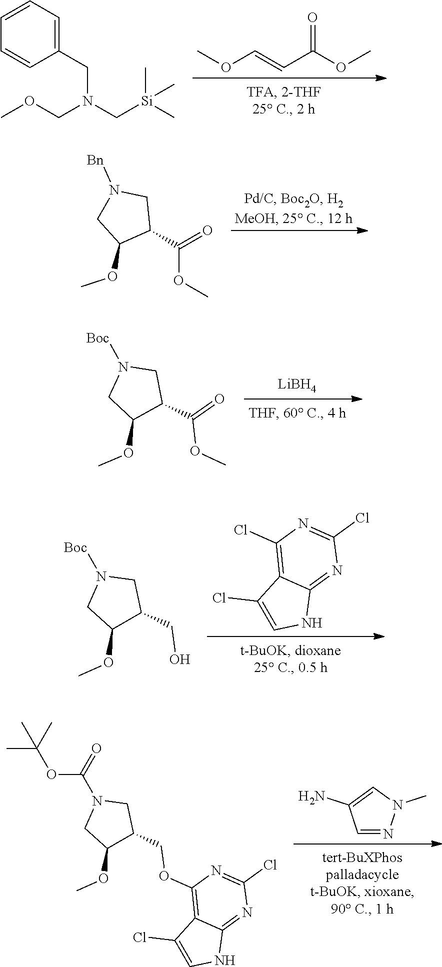

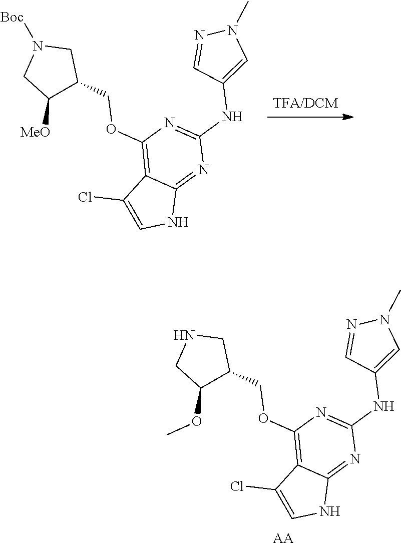

Step 1. To a solution of methyl (E)-3-methoxyprop-2-enoate (12 g, 103 mmol, 11.1 mL, 1.0 eq) in 2-methyltetrahydrofuran (180 mL) and TFA (3.1 g, 27.1 mmol, 2 mL, 0.2 eq) was added N-(methoxymethyl)-1-phenyl-N-(trimethylsilylmethyl)methanamine (49.1 g, 207 mmol, 2.0 eq) dropwise at 0° C. After addition, the reaction was allowed to warm to 25° C. and stirred for 2 h. The pH was adjusted to around 7 by progressively adding saturated NaHCO₃ solution. The mixture was diluted with EtOAc (100 mL) and brine (100 mL). The solution was separated and the organic layer was dried, filtered and concentrated to give a residue. The residue was purified by flash silica gel chromatography (PE/EtOAc=50/1 to 10/1) to give the product (3S,4R)-methyl 1-benzyl-4-methoxypyrrolidine-3-carboxylate (9.2 g, 36.3 mmol, 35% yield, 99% purity) as a yellow oil.

Step 2. To a solution of methyl (3S,4R)-1-benzyl-4-methoxy-pyrrolidine-3-carboxylate (9.2 g, 37 mmol, 99% purity, 1.0 eq) and Boc₂O (15.9 g, 73.1 mmol, 16.8 mL, 2.0 eq) in MeOH (84 mL) was added Pd/C (3.3 g, 10% purity) under N₂. The suspension was degassed under vacuum and purged with H₂ several times. The mixture was stirred under H₂ (50 psi) at 25° C. for 12 h. The mixture was filtered and the filtrate was concentrated to afford the crude product. The residue was purified by flash silica gel chromatography (PE/EtOAc=50/1 to 5/1) to yield the product (3S,4R)-1-tert-butyl 3-methyl 4-methoxypyrrolidine-1,3-dicarboxylate (7.6 g, 29.3 mmol, 80% yield) as a colorless oil. ¹H NMR: (400 MHz, CDCl₃) δ 4.15-4.12 (m, 1H), 3.73 (s, 3H), 3.66-3.61 (m, 3H), 3.45 (m, 1H), 3.36 (s, 3H), 3.08 (m, 1H), 1.46 (s, 9H).

Step 3. To a solution of (3S,4R)-1-tert-butyl 3-methyl 4-methoxypyrrolidine-1,3-dicarboxylate (7.60 g, 29.3 mmol, 1.0 eq) in THF (76 mL) was added LiBH₄ (2.4 g, 108 mmol, 3.7 eq) at 0° C. The mixture was stirred at 60° C. for 4 h. The reaction was quenched with saturated NH₄Cl (20 mL) at 0° C. and extracted with EtOAc (100 mL×2). The combined organic layer was washed with saturated aqueous brine (100 mL×2), dried, filtered and concentrated to give a residue. The residue was purified by flash silica gel chromatography (PE/EtOAc=10/1 to 1/1) to give the product (3R,4R)-tert-butyl 3-(hydroxymethyl)-4-methoxypyrrolidine-1-carboxylate (3.50 g, 15.1 mmol, 52% yield) as a colorless oil. ¹H NMR: (400 MHz, CDCl₃) δ 3.79-3.78 (m, 1H), 3.58-3.51 (m, 4H), 3.33 (m, 4H), 3.22 (m, 1H), 2.46-2.39 (m, 2H), 1.43 (s, 9H).

Step 4. A mixture of tert-butyl (3R,4R)-tert-butyl 3-(hydroxymethyl)-4-methoxypyrrolidine-1-carboxylate (4.5 g, 19.5 mmol, 1.0 eq), 2,4,5-trichloro-7H-pyrrolo[2,3-d]pyrimidine (4.3 g, 19.5 mmol, 1.0 eq) and t-BuOK (8.7 g, 77.8 mmol, 4.0 eq) in 1,4-dioxane (100 mL) was degassed and purged with N₂ for 3 times, and then the mixture was stirred at 25° C. for 0.5 h under N₂ atmosphere. The reaction was quenched with saturated NH₄Cl (200 mL) at 0° C. and extracted with EtOAc (100 mL×3). The combined organic layer was washed with saturated aqueous brine (100 mL×2), dried, filtered and concentrated to give a residue. The residue (3R,4R)-tert-butyl 3-(((2,5-dichloro-7H-pyrrolo[2,3-d]pyrimidin-4-yl)oxy)methyl)-4-methoxypyrrolidine-1-carboxylate (8.0 g) as yellow oil was used in the next step without further purification.

Step 5. A mixture of tert-butyl (3R,4R)-3-[(2,5-dichloro-7H-pyrrolo[2,3-d]pyrimidin-4-yl)oxymethyl]-4-methoxy-pyrrolidine-1-carboxylate (8.0 g, 16.5 mmol, 86% purity, 1.0 eq), 1-methylpyrazol-4-amine (2.1 g, 21.4 mmol, 1.3 eq), tert-BuXPhos palladacycle (820 mg, 1.2 mmol, 0.1 eq) and t-BuOK (5.5 g, 49.5 mmol, 3.0 eq) in 1,4-dioxane (80 mL) was degassed and purged with N₂ for 3 times, and then the mixture was stirred at 90° C. for 1 h under N₂ atmosphere. The reaction mixture was diluted with H₂O (100 mL) and extracted with EtOAc (100 mL×3). The combined organic layers were washed with brine (50 mL), dried over Na₂SO₄, filtered and concentrated under reduced pressure to give a residue. The residue was purified by flash silica gel chromatography (PE/EtOAc=50/1 to 1/1) to yield the product.

Compound (3R,4R)-tert-butyl 3-(((5-chloro-2-((1-methyl-1H-pyrazol-4-yl)amino)-7H-pyrrolo[2,3-d]pyrimidin-4-yl)oxy)methyl)-4-methoxypyrrolidine-1-carboxylate (5 g, 10.2 mmol, 62% yield, 97% purity) was obtained as a gray solid. ¹H NMR: (400 MHz, CDCl₃) δ 7.75 (s, 1H), 7.53 (s, 1H), 6.62-6.58 (m, 2H), 4.45-4.42 (m, 2H), 3.99-3.98 (m, 1H), 3.88 (s, 3H), 3.71-3.67 (m, 2H), 3.49-3.38 (m, 5H), 2.77 (m, 1H), 1.47 (s, 9H).

Step 6. A mixture of (3R,4R)-tert-butyl 3-(((5-chloro-2-((1-methyl-1H-pyrazol-4-yl)amino)-7H-pyrrolo[2,3-d]pyrimidin-4-yl)oxy)methyl)-4-methoxypyrrolidine-1-carboxylate (260 mg, 544 μmol, 1.0 eq) in TFA (0.5 mL) and DCM (2 mL) was degassed and purged with N₂ for 3 times, and then the mixture was stirred at 25° C. for 1 h under N₂ atmosphere. The mixture was evaporated to afford the crude product 5-chloro-4-(((3R,4R)-4-methoxypyrrolidin-3-yl)methoxy)-N-(1-methyl-1H-pyrazol-4-yl)-7H-pyrrolo[2,3-d]pyrimidin-2-amine AA (180 mg, 457 μmol, 96% purity) as a yellow solid. The residue was used in the next step without further purification.

### Compound AB: 2-((3-(3-chloro-4-methylphenyl)-1-((2-(2,6-dioxopiperidin-3-yl)-1-oxoisoindolin-5-yl)methyl)ureido)methyl)acrylic acid

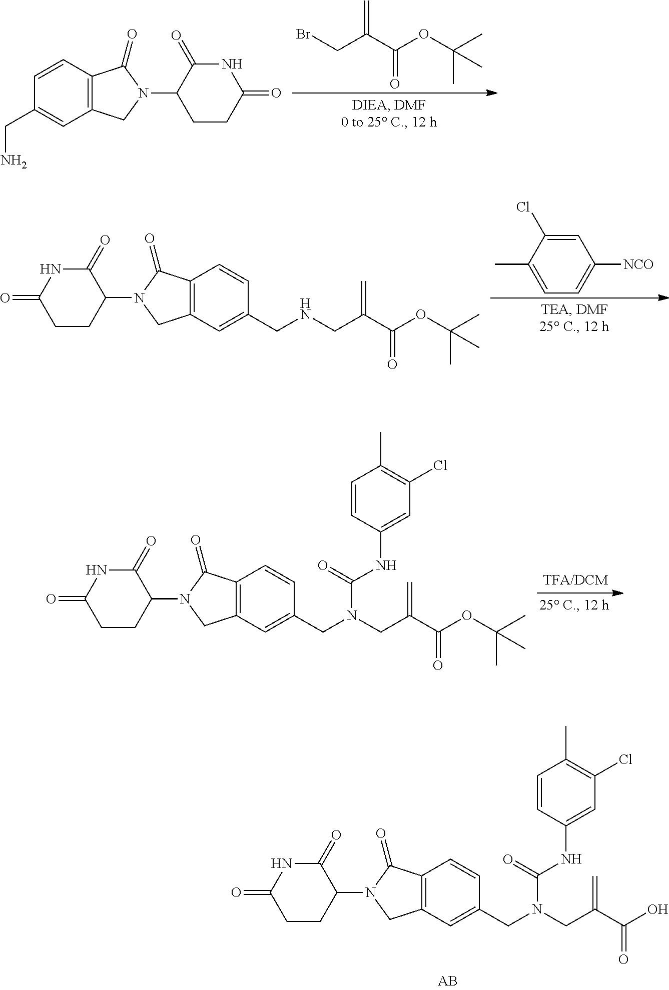

Step 1. To a solution of 3-[5-(aminomethyl)-1-oxo-isoindolin-2-yl]piperidine-2,6-dione (500 mg, 1.8 mmol, 1.0 eq) and DIPEA (709 mg, 5.5 mmol, 956 μL, 3.0 eq) in DMF (10 mL) was drop-wise added the solution of tert-butyl 2-(bromomethyl)prop-2-enoate (405 mg, 1.8 mmol, 1.0 eq) in DMF (3 mL) at 0° C. Then, the reaction mixture was stirred at 25° C. for 12 h. The resultant mixture was filtered and the filtrated was concentrated under vacuum. The mixture was purified by semi-preparative reverse phase HPLC (18-48% acetonitrile+0.225% formic acid in water, 20 min). The compound tert-butyl 2-[[[2-(2,6-dioxo-3-piperidyl)-1-oxo-isoindolin-5-yl]methylamino]methyl]prop-2-enoate (310 mg, 750 μmol, 41.0% yield) was obtained as a white solid. H NMR: (400 MHz, DMSO-d6) δ=10.98 (s, 1H), 7.69 (d, J=7.8 Hz, 1H), 7.60 (s, 1H), 7.50 (d, J=7.9 Hz, 1H), 6.11 (s, 1H), 5.81 (d, J=1.3 Hz, 1H), 5.11 (d, J=5.1, 13.3 Hz, 1H), 4.49-4.42 (m, 1H), 4.34-4.27 (m, 1H), 3.89 (s, 2H), 3.37 (s, 2H), 2.96-2.88 (m, 1H), 2.68-2.52 (m, 1H), 2.40-2.32 (m, 1H), 2.03-1.96 (m, 1H), 1.44 (s, 9H).

Step 2. To a solution of 2-chloro-4-isocyanato-1-methyl-benzene (188 mg, 1.1 mmol, 1.5 eq) and tert-butyl 2-[[[2-(2,6-dioxo-3-piperidyl)-1-oxo-isoindolin-5-yl]methylamino]methyl]prop-2-enoate (310 mg, 750 μmol, 1.0 eq) in DMF (5 mL) was added TEA (228 mg, 2.2 mmol, 313 μL, 3.0 eq). The reaction mixture was stirred at 25° C. for 12 h. The resultant mixture was adjusted to pH=6 with formic acid. The residue was purified by prep-HPLC column: Phenomenex luna C8 250×50 mm×5 um; mobile phase: [water(FA)-ACN]; B %: 42%-72%, 25 min). Compound tert-butyl 2-[[(3-chloro-4-methyl-phenyl)carbamoyl-[[2-(2,6-dioxo-3-piperidyl)-1-oxo-isoindolin-5-yl]methyl]amino]methyl]prop-2-enoate (280 mg, 482 μmol, 64% yield) was obtained as a white solid. ¹H NMR: (400 MHz, CD₃Cl) 6=7.81-7.79 (d, J=8.0 Hz, 1H), 7.53 (d, J=2.0 Hz, 1H), 7.48-7.46 (d, J=8.0 Hz, 1H), 7.20 (m, 2H), 6.27 (s, 1H), 5.73 (s, 1H), 5.19-5.14 (m, 1H), 4.73 (s, 2H), 4.55-4.49 (m, 2H), 4.22 (m, 2H), 2.96-2.92 (m, 1H), 2.82-2.80 (m, 1H), 2.49 (m, 1H), 2.31 (s, 3H), 2.20-2.18 (m, 1H), 1.52 (s, 9H).

Step 3. To a solution of tert-butyl 2-[[(3-chloro-4-methyl-phenyl)carbamoyl-[[2-(2,6-dioxo-3-piperidyl)-1-oxo-isoindolin-5-yl]methyl]amino]methyl]prop-2-enoate (130 mg, 224 μmol, 1.0 eq) in DCM (2 mL) was added TFA (3.1 g, 27.0 mmol, 2.0 mL, 121 eq) and the reaction mixture was stirred at 25° C. for 12 h. The resultant mixture was concentrated under vacuum. The crude product 2-[[(3-chloro-4-methyl-phenyl)carbamoyl-[[2-(2,6-dioxo-3-piperidyl)-1-oxo-isoindolin-5-yl]methyl]amino]methyl]prop-2-enoic acid AB (140 mg, 219 μmol, 98% yield, TFA) was obtained as a brown oil and used into the next step without further purification.

### Compound AC: 2-((4-chloro-2-(3-((2-(2,6-dioxopiperidin-3-yl)-1-oxoisoindolin-5-yl)methyl)ureido)phenoxy)methyl)acrylic acid

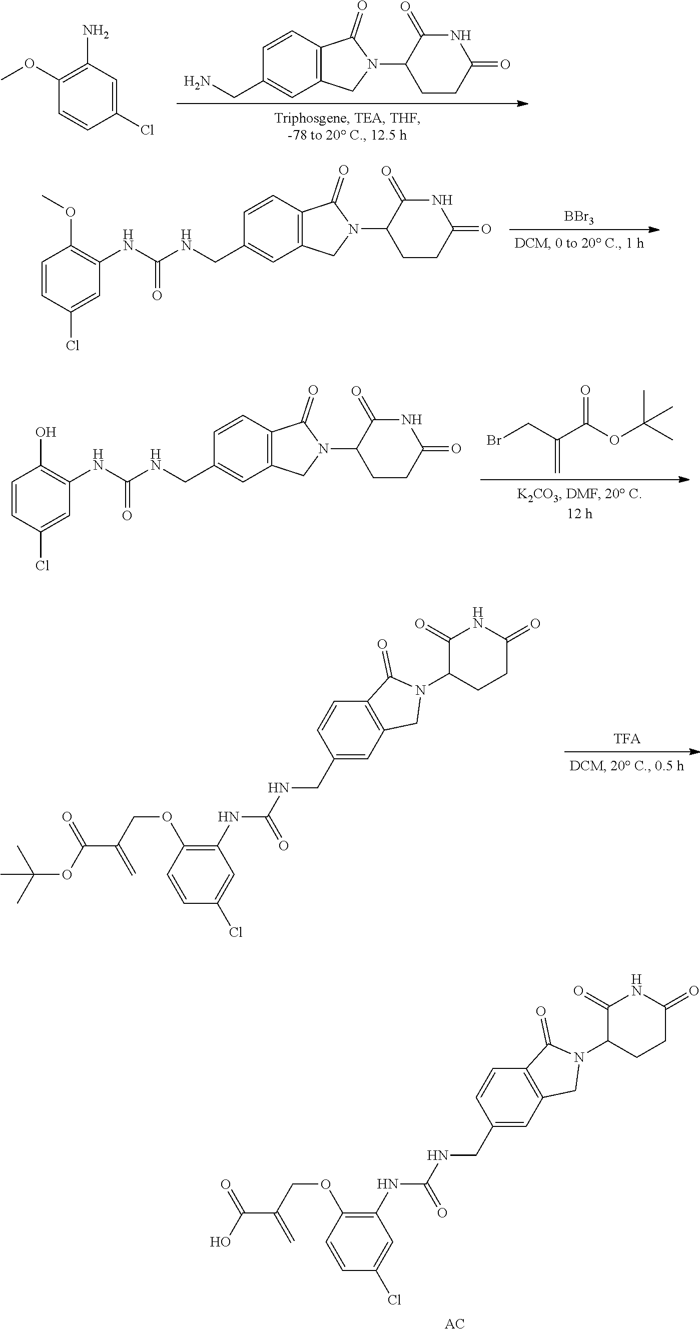

Step 1. To a solution of triphosgene (363 mg, 1.22 mmol, 0.8 eq) in THF (30 mL) was added 5-chloro-2-methoxyaniline (200 mg, 1.27 mmol, 1.0 eq) and TEA (1.28 g, 12.7 mmol, 1.77 mL, 10.0 eq) in THF (10 mL) slowly at −78° C., and then it was stirred for 0.5 h. 3-(5-(Aminomethyl)-1-oxoisoindolin-2-yl)piperidine-2,6-dione (0.393 g, 1.27 mmol, 1.0 eq, HCl salt) was added to the mixture and then it was stirred at 20° C. for 12 h. The reaction mixture was diluted with H₂O 15 mL and extracted with EA 30 mL (15 mL×2). The combined organic layers were dried over Na₂SO₄, filtered and concentrated under reduced pressure to give a residue. The residue was purified by column chromatography (SiO₂, Petroleum ether/Ethyl acetate=10/1 to 1/1) to afford 1-(5-chloro-2-methoxyphenyl)-3-((2-(2,6-dioxopiperidin-3-yl)-1-oxoisoindolin-5-yl)methyl)urea (220 mg, 481 μmol, 38% yield) as a white solid. ¹H NMR: (400 MHz, CDCl₃): δ 8.23-8.20 (m, 1H), 7.98-7.93 (m, 1H), 7.80-7.75 (m, 1H), 7.51-7.47 (m, 1H), 7.44-7.38 (m, 1H), 7.05-7.00 (m, 1H), 6.93 (d, J=2.5, 8.6 Hz, 1H), 6.75 (d, J=8.8 Hz, 1H), 5.36-5.26 (m, 1H), 5.24-5.17 (m, 1H), 4.58-4.53 (m, 2H), 4.43 (s, 1H), 4.29 (s, 1H), 3.84-3.80 (m, 3H), 3.77-3.70 (m, 2H), 2.96-2.90 (m, 1H), 2.82 (s, 1H), 2.40-2.31 (m, 1H), 2.20-2.25 (m, J=2.2, 5.0, 10.5 Hz, 1H).

Step 2. To a solution of 1-(5-chloro-2-methoxyphenyl)-3-((2-(2,6-dioxopiperidin-3-yl)-1-oxoisoindolin-5-yl)methyl)urea (220 mg, 481 μmol, 1.0 eq) in DCM (4 mL) was added BBr₃ (603 mg, 2.41 mmol, 231 μL, 5.0 eq) at 0° C. The mixture was stirred at 20° C. for 1 h. The reaction mixture was quenched by addition H₂O (5 mL) at 0° C., the reaction mixture was concentrated under reduced pressure to remove DCM. The residue was filtered to give 1-(5-chloro-2-hydroxyphenyl)-3-((2-(2,6-dioxopiperidin-3-yl)-1-oxoisoindolin-5-yl)methyl)urea (120 mg, 270 μmol, 56% yield) as a white solid.

Step 3. To a solution of 1-(5-chloro-2-hydroxyphenyl)-3-((2-(2,6-dioxopiperidin-3-yl)-1-oxoisoindolin-5-yl)methyl)urea (100 mg, 225 μmol, 1.0 eq), tert-butyl 2-(bromomethyl) acrylate (J. Med. Chem., 2021, 64, 1835, 49.9 mg, 225 μmol, 1.0 eq) in DMF (1.5 mL) was added K₂CO₃ (93.6 mg, 677 μmol, 3.0 eq). The mixture was stirred at 20° C. for 12 h. The reaction mixture was filtered and concentrated under reduced pressure to give a residue. The residue was purified by prep-HPLC (column: Phenomenex C18 75×30 mm×3 um; mobile phase: [water (FA)-ACN]; B %: 40%-70%, 7 min) to afford tert-butyl 2-((4-chloro-2-(3-((2-(2,6-dioxopiperidin-3-yl)-1-oxoisoindolin-5-yl)methyl)ureido)phenoxy) methyl)acrylate (50 mg, 81 μmol, 36% yield) as a white solid.

Step 4. To a solution of tert-butyl 2-((4-chloro-2-(3-((2-(2,6-dioxopiperidin-3-yl)-1-oxoisoindolin-5-yl)methyl)ureido)phenoxy)methyl)acrylate (50 mg, 120 μmol, 1.0 eq) in DCM (1 mL) was added TFA (0.5 mL). The mixture was stirred at 20° C. for 0.5 h. The reaction mixture was filtered and concentrated under reduced pressure to give 2-((4-chloro-2-(3-((2-(2,6-dioxopiperidin-3-yl)-1-oxoisoindolin-5-yl)methyl)ureido)phenoxy)methyl)acrylic acid AC (56 mg, TFA salt) as a white solid.

### Compound AD: 2-((3-chloro-5-(3-((2-(2,6-dioxopiperidin-3-yl)-1-oxoisoindolin-5-yl)methyl)ureido)phenoxy)methyl)acrylic acid

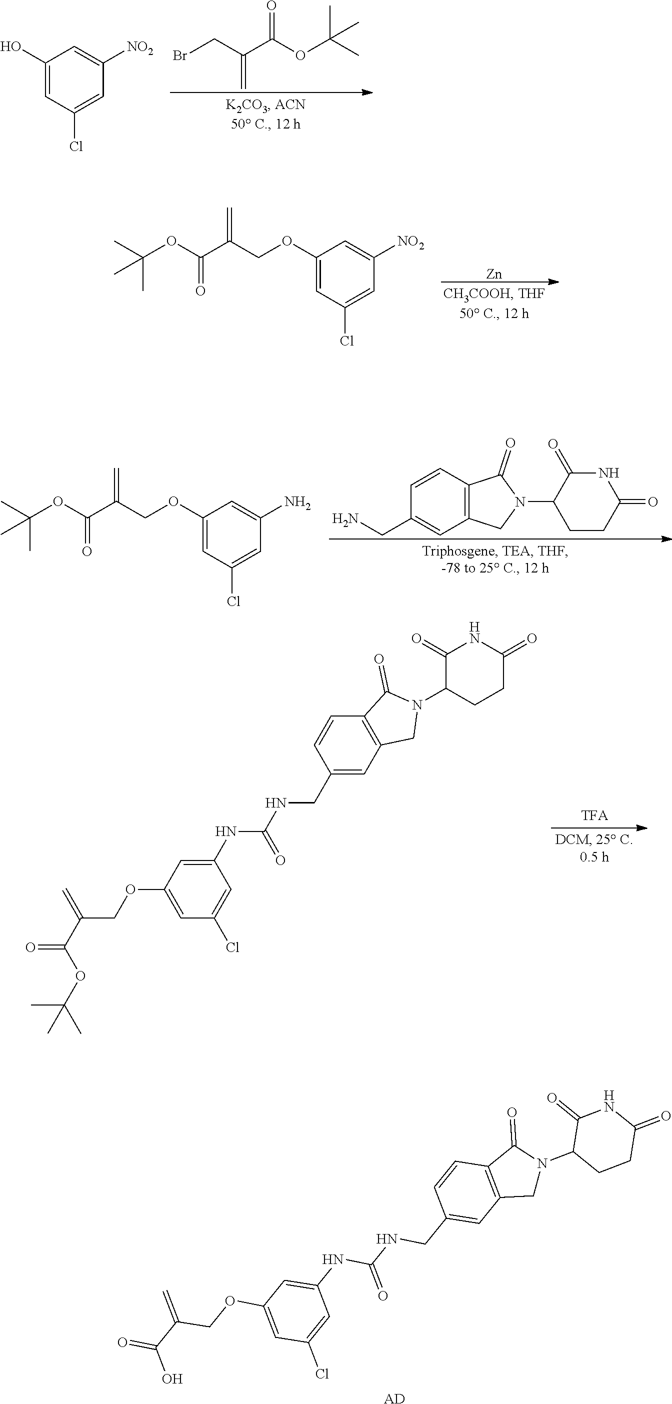

Step 1. To a solution of 3-chloro-5-nitro-phenol (4.01 g, 23.1 mmol, 1.0 eq) in ACN (120 mL) was added tert-butyl 2-(bromomethyl)prop-2-enoate (5.11 g, 23.1 mmol, 1.0 eq) and K₂CO₃ (9.58 g, 69.3 mmol, 3.0 eq). The mixture was stirred at 50° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by column chromatography (SiO₂, Petroleum ether/Ethyl acetate=1/0 to 20/1) to give tert-butyl 2-[(3-chloro-5-nitro-phenoxy)methyl]prop-2-enoate (5.27 g, 16.80 mmol, 72.70% yield) as a yellow oil. ¹H NMR: (DMSO-d₆, 400 MHz) 7.87 (t, J=1.8 Hz, 1H), 7.76-7.71 (m, 1H), 7.66-7.54 (m, 1H), 6.28-6.20 (m, 1H), 6.05-5.91 (m, 1H), 4.92-4.79 (m, 2H), 1.47-1.44 (m, 9H).

Step 2. To a solution of tert-butyl 2-[(3-chloro-5-nitro-phenoxy)methyl]prop-2-enoate (3.00 g, 9.56 mmol, 1.0 eq) in CH₃COOH (15 mL) and THF (30 mL) was added Zn (2.67 g, 47.8 mmol, 5.0 eq). The mixture was stirred at 50° C. for 12 h. The reaction mixture was filtered and the filtrate was concentrated to afford crude product. The residue was purified by prep-HPLC (column: Waters Xbridge C18 150×50 mm×10 um; mobile phase: [water(NH₄HCO₃)-ACN]; B %: 42%-72%, 10 min). Compound tert-butyl 2-[(3-amino-5-chloro-phenoxy)methyl]prop-2-enoate (1.00 g, 3.49 mmol, 36% yield) was obtained as a white solid. ¹H NMR: (CD₃SOCD₃, 400 MHz) 6.20 (t, J=1.8 Hz, 1H), 6.18-6.16 (m, 1H), 6.14-6.11 (m, 1H), 6.10-6.06 (m, 1H), 5.88-5.84 (m, 1H), 4.67-4.50 (m, 2H), 1.48-1.43 (m, 9H).

Step 3. To a solution of Triphosgene (0.155 g, 522 μmol, 0.8 eq) and TEA (1.78 g, 17.6 mmol, 2 mL, 5.0 eq) in THF (40 mL) was slowly added tert-butyl 2-[(3-amino-5-chloro-phenoxy)methyl]prop-2-enoate (1.00 g, 3.52 mmol, 1.0 eq) in THF (10 mL) at −78° C., and then it was stirred for 0.5 h. 3-[5-(aminomethyl)-1-oxo-isoindolin-2-yl]piperidine-2,6-dione (1.09 g, 3.52 mmol, 1.0 eq, HCl) was added to the mixture and then it was stirred at 25° C. for 11.5 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by column chromatography (SiO₂, Petroleum ether/Ethyl acetate=6/1 to 0/1). Compound tert-butyl 2-[[3-chloro-5-[[2-(2,6-dioxo-3-piperidyl)-1-oxo-isoindolin-5-yl]methylcarbamoylamino]phenoxy]methyl]prop-2-enoate (375 mg, 579 μmol, 16% yield) was obtained as a yellow oil. ¹H NMR: (DMSO-d₆, 400 MHz) 10.98 (s, 1H), 8.96-8.81 (m, 1H), 7.73-7.65 (m, 1H), 7.53-7.50 (m, 1H), 7.47-7.41 (m, 1H), 7.20-7.16 (m, 1H), 6.99-6.96 (m, 1H), 6.88-6.82 (m, 1H), 6.63-6.57 (m, 1H), 6.21-6.15 (m, 1H), 5.91-5.82 (m, 1H), 5.13-5.05 (m, 1H), 4.69-4.63 (m, 2H), 4.45-4.24 (m, 4H), 2.98-2.85 (m, 1H), 2.64-2.57 (m, 1H), 2.41-2.32 (m, 1H), 2.04-1.97 (m, 1H), 1.45-1.42 (m, 9H).

Step 4. To a solution of tert-butyl 2-[[3-chloro-5-[[2-(2,6-dioxo-3-piperidyl)-1-oxo-isoindolin-5-yl]methylcarbamoylamino]phenoxy]methyl]prop-2-enoate (290 mg, 497 μmol, 1.0 eq) in DCM (4 mL) was added TFA (2 mL). The mixture was stirred at 25° C. for 0.5 h. The reaction mixture was concentrated to afford 2-[[3-chloro-5-[[2-(2,6-dioxo-3-piperidyl)-1-oxo-isoindolin-5-yl]methylcarbamoylamino]phenoxy]methyl]prop-2-enoic acid AD (319 mg, TFA salt) as a yellow oil and it was used into the next step without further purification.

### Compound AE: 2-((4-((3-(5-((3-(3-chloro-4-methylphenyl)ureido)methyl)-1-oxoisoindolin-2-yl)-2,6-dioxopiperidin-1-yl)methyl)phenoxy)methyl)acrylic acid

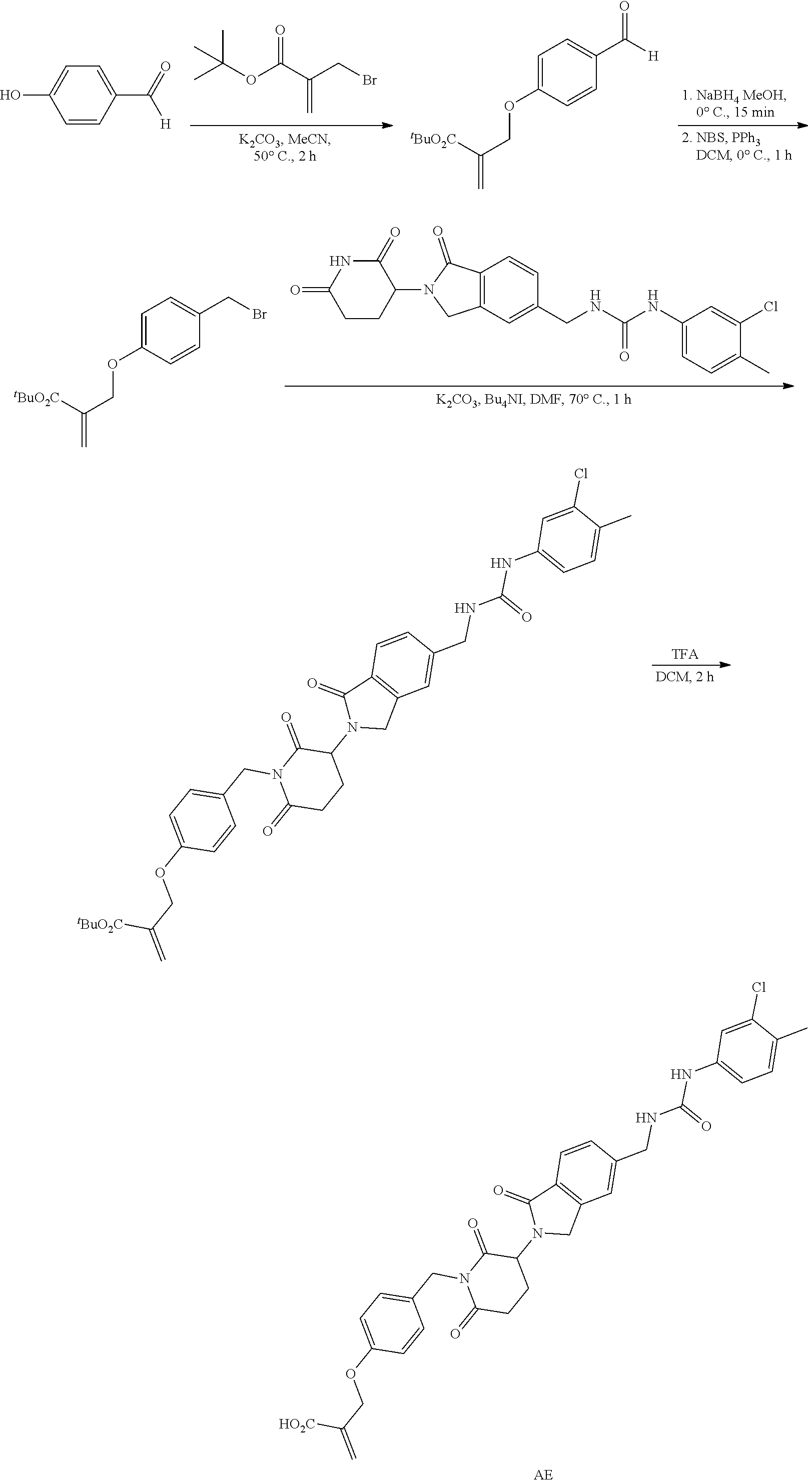

Step 1. To a suspension of tert-butyl 2-(bromomethyl)acrylate (0.62 g, 2.80 mmol) and K₂CO₃ (1.16 g, 8.41 mmol) in MeCN (14 mL) was added 4-hydroxybenzaldehyde (0.342 g, 2.80 mmol) at rt. The reaction mixture was heated to 50° C. for 4 h and then cooled to rt. The volatiles were evaporated under reduced pressure. Water (20 mL) and EtOAc (20 mL) were added, and the layers were separated. The aqueous phase was extracted with EtOAc (2×20 mL). The organic layers were combined, dried over Na₂SO₄, filtered, and concentrated under reduced pressure. The crude product was purified by column chromatography on silica gel using a gradient of 0-20% EtOAc in hexane to afford tert-butyl 2-((4-formylphenoxy)methyl)acrylate (0.632 g, 86%) as a solid. LC-MS Method A: MS (ES⁺): Rₜ=1.46 mi, m/z=no ionization.

Step 2. Step 1: to a solution of tert-butyl 2-((4-formylphenoxy)methyl)acrylate (0.632 g, 2.41 mmol) in MeOH (27 mL) cooled to 0° C. was added NaBH4 (0.0684 g, 1.81 mmol). The reaction mixture was stirred at 0° C. for 20 mn. Water (10 mL) was added at 0° C. The volatiles were evaporated under reduced pressure. Water (50 mL) and EtOAc (100 mL) were added, stirred for 1 h at rt and then the layers were separated. The aqueous layer was extracted with EtOAc (3×100 mE). The organic layers were combined, dried over Na₂SO₄, filtered, and concentrated under reduced pressure to afford the benzylic alcohol intermediate as a solid. To a solution of the crude benzylic alcohol in DCM (21 mL) cooled to 0° C. were sequentially added PPh₃ (0.950 g, 2.19 mmol) and NBS (0.643 g, 2.19 mmol). The reaction mixture was stirred at 0° C. for 1 h. The reaction mixture was neutralized with a saturated solution of NaHCO₃, and the layers were separated. The aqueous layer was extracted with DCM (3×30 mL). The organic layers were combined, dried over Na₂SO₄, filtered, and concentrated under reduced pressure. The crude product was purified by column chromatography on silica gel using a gradient of 0-10% EtOAc in hexane to afford tert-butyl 2-((4-(bromomethyl)phenoxy)methyl)acrylate (330 mg, 42%) as a solid. ¹H NMR (CHCl₃-d, 400 MHz): δ_{H} 7.32 (2H, d, J=8.5 Hz), 6.89 (2H, d, J=8.5 Hz), 6.29 (1H, d, J=1.6 Hz), 5.89 (1H, d, J=1.8 Hz), 4.71 (2H, s), 4.49 (2H, s), 1.51 (9H, s).

Step 3. A solution of tert-butyl 2-((4-(bromomethyl)phenoxy)methyl)acrylate (133 mg, 407 μmol) in DMF (2.7 mL) was added to a mixture of 1-(3-chloro-4-methylphenyl)-3-((2-(2,6-dioxopiperidin-3-yl)-1-oxoisoindolin-5-yl)methyl)urea (65 mg, 0.147 mmol) and K₂CO₃ (40.8 mg, 0.295 mmol). Bu₄NI (16.5 mg, 0.0442 mmol) was then added. The reaction mixture was heated to 70° C. for 1 h and then cooled to rt. The volatiles were evaporated under reduced pressure. Water (20 mL) and EtOAc (20 mL) were added, and the layers were separated. The aqueous layer was extracted with EtOAc (3×20 mL). The organic layers were combined, dried over Na₂SO₄ and concentrated under reduced pressure. The crude product was purified by column chromatography on silica gel using a gradient of 0-1% ⁱPrOH in EtOAc to afford tert-butyl 2-((4-((3-(5-((3-(3-chloro-4-methylphenyl)ureido)methyl)-1-oxoisoindolin-2-yl)-2,6-dioxopiperidin-1-yl)methyl)phenoxy)methyl)acrylate (82 mg, 81%) as a solid. LC-MS Method B: MS (ES⁺): Rₜ=1.80 min, m/z=709.3 [M+Na]⁺

Step 4. A solution of tert-butyl 2-((4-((3-(5-((3-(3-chloro-4-methylphenyl) ureido)methyl)-1-oxoisoindolin-2-yl)-2,6-dioxopiperidin-1-yl)methyl)phenoxy)methyl)acrylate (82.0 mg, 0.119 mmol) in a mixture of DCM (5.0 mL) and TFA (0.547 mL) was stirred at rt for 1 h. The volatiles were evaporated under reduced pressure. The crude product was co-evaporated with toluene (3×10 mL) to afford 2-((4-((3-(5-((3-(3-chloro-4-methylphenyl)ureido)methyl)-1-oxoisoindolin-2-yl)-2,6-dioxopiperidin-1-yl)methyl)phenoxy)methyl)acrylic acid AE (70 mg, 93%) as a solid. LC-MS Method C: MS (ES⁺): Rₜ=3.35 min, m/z=631.3 [M+H]⁺¹H NMR (DMSO-d₆, 400 MHz): δ_{H} 8.75 (1H, s), 7.67-7.71 (2H, m), 7.52 (1H, s), 7.44 (1H, d, J=7.9 Hz), 7.11-7.19 (4H, m), 6.88 (2H, d, J=8.2 Hz), 6.79 (1H, t, J=6.4 Hz), 6.23 (1H, s), 5.91 (1H, s), 5.24 (1H, dd, J=13.3, 5.0 Hz), 4.67-4.80 (4H, m), 4.47 (1H, d, J=17.2 Hz), 4.41 (2H, d, J=5.5 Hz), 4.28 (1H, d, J=17.2 Hz), 3.12-3.04 (1H, m), 2.81-2.77 (1H, m), 2.47-2.33 (1H, m), 2.23 (3H, s), 2.07-2.01 (1H, m).

### Compound AF: 2-(6-chloro-8-fluoro-4-(piperazin-1-yl)quinazolin-7-yl)-3-fluorophenol

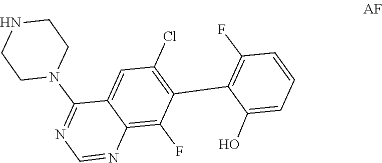 [SMILES: Oc1cccc(F)c1-c1c(Cl)cc2c(N3CCNCC3)ncnc2c1F]

Compound AF was prepared based on procedures described in WO 2017/100546.

### Compound AG: 2-((5-chloro-2-(3-((2-(2,6-dioxopiperidin-3-yl)-1-oxoisoindolin-5-yl)methyl)ureido)-4-methoxyphenoxy)methyl)acrylic acid

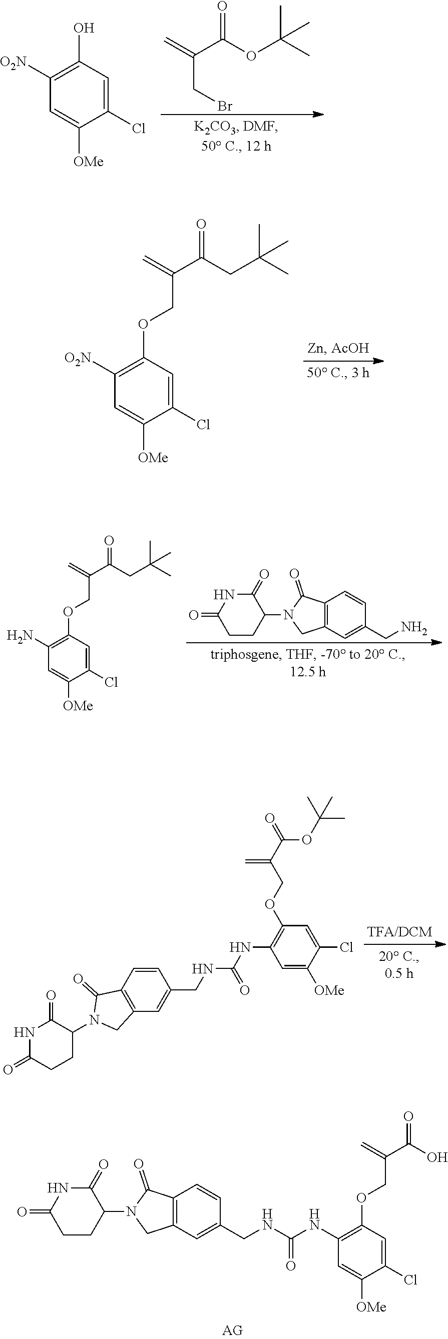

Step 1. A mixture of 5-chloro-4-methoxy-2-nitrophenol (where 5-chloro-4-methoxy-2-nitrophenol was prepared based on procedures described in WO2018/37223, 1.80 g, 8.84 mmol, 1.0 eq), tert-butyl 2-(bromomethyl)acrylate (1.95 g, 8.84 mmol, 1.0 eq) and K₂CO₃ (3.67 g, 26.53 mmol, 3.0 eq) in DMF (15 mL) was stirred at 50° C. for 12 h. The reaction mixture was filtered and concentrated under reduced pressure to give a residue. The residue was purified by prep-HPLC (column: Waters Xbridge C18 150×50 mm×10 um; mobile phase: [water (NH₄HCO₃)-ACN]; B %: 47%-77%, 10 min) to give tert-butyl 2-((5-chloro-4-methoxy-2-nitrophenoxy)methyl)acrylate (1.6 g, 4.65 mmol, 52% yield) as a yellow solid. ¹HNMR (400 MHz, DMSO): δ 7.69 (s, 1H), 7.63 (s, 1H), 6.22 (s, 1H), 5.95 (d, J=1.2 Hz, 1H), 4.87 (s, 2H), 3.88 (s, 3H), 1.43 (s, 9H).

Step 2. To a solution of tert-butyl 2-((5-chloro-4-methoxy-2-nitrophenoxy)methyl)acrylate (0.5 g, 1.45 mmol, 1.0 eq) in THF (10 mL) was added Zn (470 mg, 7.27 mmol, 5.0 eq) and AcOH (437 mg, 7.27 mmol, 5.0 eq). The mixture was stirred at 50° C. for 3 h. The reaction mixture was filtered and concentrated under reduced pressure to give a residue. The residue was purified by prep-TLC (SiO₂, Petroleum ether/Ethyl acetate=3:1). tert-butyl 2-((2-amino-5-chloro-4-methoxyphenoxy)methyl)acrylate (240 mg, 764 μmol, 52% yield) was obtained as a brown gum. ¹H NMR (400 MHz, CDCl₃): δ 6.86 (s, 1H), 6.38 (s, 1H), 6.31 (d, J=1.1 Hz, 1H), 5.88-5.85 (m, 1H), 4.65 (s, 2H), 4.08-3.65 (m, 5H), 1.53 (s, 9H).

Step 3. To a solution of triphosgene (185 mg, 0.62 mmol, 0.8 eq) in THF (20 mL) was added tert-butyl 2-((2-amino-5-chloro-4-methoxyphenoxy)methyl)acrylate (240 mg, 764 μmol, 1.0 eq) and TEA (773 mg, 7.65 mmol, 1.0 mL, 10 eq) in THF (5.0 mL) drop-wisely at −70° C. The mixture was stirred at −70° C. for 0.5 h. Then 3-(5-(aminomethyl)-1-oxoisoindolin-2-yl)piperidine-2,6-dione (355 mg, 962 μmol, 1.26 eq, CH₃SO₃H salt) was added into the reaction mixture and stirred at 25° C. for 12 h. The reaction mixture was filtered and concentrated under reduced pressure to give a residue. The residue was purified by prep-TLC (SiO₂, DCM: MeOH=10:1) to give tert-butyl 2-((5-chloro-2-(3-((2-(2,6-dioxopiperidin-3-yl)-1-oxoisoindolin-5-yl)methyl)ureido)-4-methoxyphenoxy)methyl)acrylate (70 mg, 114 μmol, 14% yield) as an off-white solid.

Step 4. To a solution of tert-butyl 2-((5-chloro-2-(3-((2-(2,6-dioxopiperidin-3-yl)-1-oxoisoindolin-5-yl)methyl)ureido)-4-methoxyphenoxy)methyl)acrylate (70 mg, 114 μmol, 1.0 eq) in DCM (1 mL) was added TFA (0.5 mL). The mixture was stirred at 20° C. for 0.5 h. The reaction mixture was concentrated under reduced pressure to give desired product 2-((5-chloro-2-(3-((2-(2,6-dioxopiperidin-3-yl)-1-oxoisoindolin-5-yl)methyl)ureido)-4-methoxyphenoxy) methyl)acrylic acid AF (63 mg, 113 μmol, 99% yield) as a brown gum and it was used into the next step without further purification.

### Compound AH: 2-((3-(3-((2-(2,6-dioxopiperidin-3-yl)-1-oxoisoindolin-5-yl)methyl)ureido)-5-(trifluoromethyl)phenoxy)methyl)acrylic acid

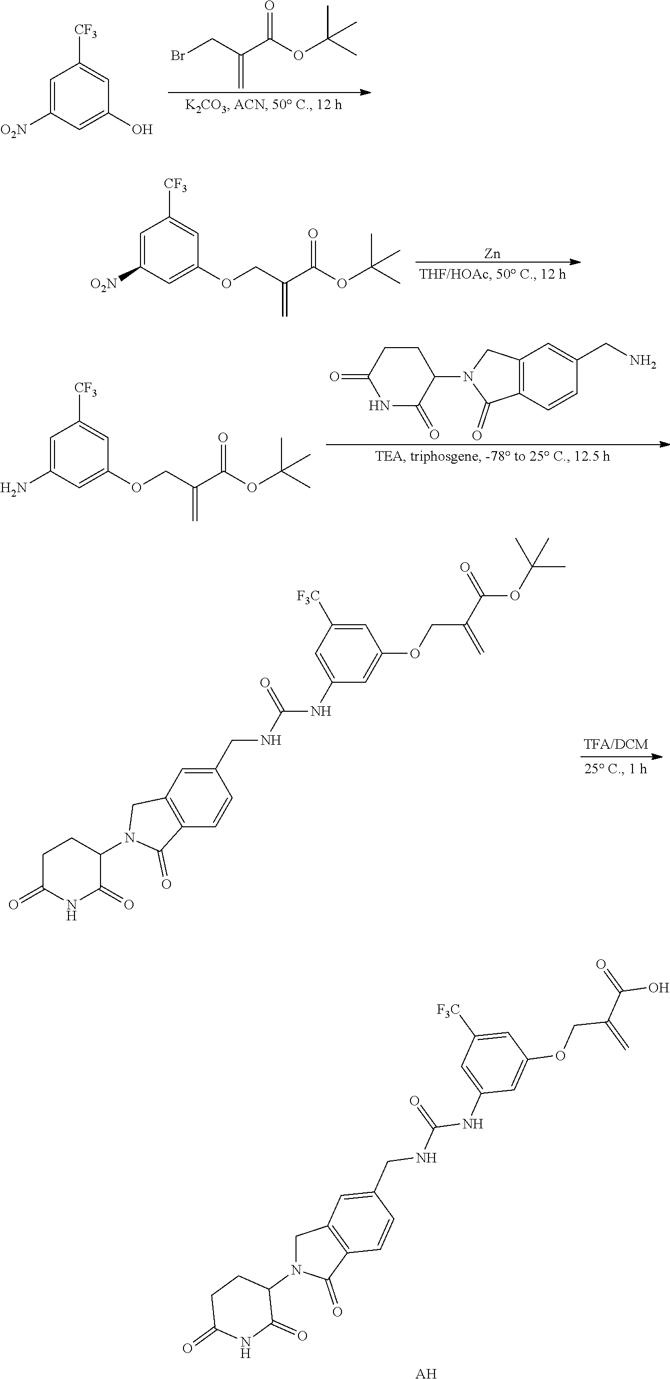

Step 1. To a solution of 3-nitro-5-(trifluoromethyl)phenol (2.0 g, 9.7 mmol, 1.0 eq) and tert-butyl 2-(bromomethyl)prop-2-enoate (2.1 g, 9.7 mmol, 1.0 eq) in ACN (15 mL) was added K₂CO₃ (4.0 g, 29.0 mmol, 3.0 eq). The mixture was stirred at 50° C. for 12 h. The reaction mixture was filtered and concentrated under reduced pressure to give a residue. The residue was purified by column chromatography (SiO₂, Petroleum ether/Ethyl acetate=10/1 to 5/1). Compound tert-butyl 2-[[3-nitro-5-(trifluoromethyl)phenoxy]methyl]prop-2-enoate (1.3 g, 3.7 mmol, 39% yield) was obtained as a white solid. ¹H NMR (400 MHz, CDCl₃) δ 8.11 (s, 2H), 7.96 (t, J=2.1 Hz, 2H), 7.51 (s, 2H), 6.41-6.35 (m, 2H), 5.97-5.92 (m, 2H), 4.85 (s, 5H), 1.70-1.57 (m, 1H), 1.52-1.37 (m, 2H).

Step 2. To a solution of tert-butyl 2-[[3-nitro-5-(trifluoromethyl)phenoxy]methyl]prop-2-enoate (1.3 g, 3.7 mmol, 1.0 eq) in THF (10 mL) and HOAc (5 mL) was added Zn (1.1 g, 18.7 mmol, 5.0 eq). The mixture was stirred at 50° C. for 12 h. The reaction mixture was filtered and concentrated under reduced pressure to give a residue. The residue was purified by prep-HPLC (column: Waters Xbridge C18 150×50 mm×10 um; mobile phase: [water(NH₄HCO₃)-ACN]; B %: 44%-74%, 10 min). tert-butyl 2-[[3-amino-5-(trifluoromethyl) phenoxy]methyl]prop-2-enoate (500 mg, 1.6 mmol, 42% yield) was obtained as a white solid. ¹H NMR (400 MHz, CDCl₃) δ 7.27 (s, 1H), 6.57 (s, 2H), 6.53 (s, 2H), 6.39 (s, 2H), 6.34-6.27 (m, 2H), 5.93-5.87 (m, 2H), 4.70 (s, 4H), 4.17-4.09 (m, 2H), 3.85 (br s, 4H), 2.05 (s, 2H), 1.61-1.56 (m, 1H), 1.50-1.44 (m, 1H), 1.31-1.22 (m, 3H).

Step 3. To a solution of triphosgene (175 mg, 591 μmol, 0.7 eq) in THF (10 mL) was added TEA (797 mg, 7.9 mmol, 1.1 mL, 10.0 eq) and tert-butyl 2-[[3-amino-5-(trifluoromethyl) phenoxy]methyl]prop-2-enoate (250 mg, 788 μmol, 1.0 eq) in THF (5 mL) at −78° C. The mixture was stirred at −78° C. for 0.5 h and then 3-[5-(aminomethyl)-1-oxo-isoindolin-2-yl]piperidine-2,6-dione (215 mg, 695 μmol, 0.9 eq, HCl salt) was added. The reaction mixture was stirred at 25° C. for 12 h. The reaction mixture was partitioned between water (50 mL) and EA (50 mL). The organic phase was separated, washed with ethyl acetate (10 mL×3), filtered and concentrated under reduced pressure to give a residue. The residue was purified by column chromatography (SiO₂, Petroleum ether/Ethyl acetate=3/1 to 0/1). Compound tert-butyl 2-[[3-[[2-(2,6-dioxo-3-piperidyl)-1-oxo-isoindolin-5-yl]methylcarbamoylamino]-5-(trifluoromethyl) phenoxy]methyl]prop-2-enoate (180 mg, 292 μmol, 37% yield) was obtained as a white solid. ¹H NMR: (400 MHz, CD₃OD) δ 7.77 (d, J=7.9 Hz, 1H), 7.56 (s, 1H), 7.50 (d, J=7.8 Hz, 1H), 7.33 (br d, J=2.7 Hz, 2H), 6.81 (s, 1H), 6.28 (s, 1H), 5.91 (d, J=1.1 Hz, 1H), 5.18-5.11 (m, 1H), 4.74 (s, 2H), 4.57-4.43 (m, 4H), 4.14-4.07 (m, 1H), 3.00-2.84 (m, 1H), 2.83-2.74 (m, 1H), 2.55-2.42 (m, 1H), 2.22-2.13 (m, 1H), 2.02 (s, 1H), 1.50 (s, 9H), 1.32-1.17 (m, 2H).

Step 4. To a solution of tert-butyl 2-[[3-[[2-(2,6-dioxo-3-piperidyl)-1-oxo-isoindolin-5-yl]methylcarbamoylamino]-5-(trifluoromethyl)phenoxy]methyl]prop-2-enoate (90 mg, 146 μmol, 1.0 eq) in DCM (1 mL) was added TFA (0.5 mL). The mixture was stirred at 25° C. for 0.5 h. The reaction mixture was concentrated under reduced pressure to give a residue. Compound 2-[[3-[[2-(2,6-dioxo-3-piperidyl)-1-oxo-isoindolin-5-yl]methylcarbamoylamino]-5-(trifluoromethyl)phenoxy]methyl]prop-2-enoic acid AH (98 mg, 145 μmol, TFA salt) was obtained as a yellow oil and it was used directly for the next step.

### Compound AI: 2-((3-(3-((2-(2,6-dioxopiperidin-3-yl)-1-oxoisoindolin-5-yl)methyl)ureido)phenoxy)methyl)acrylic acid

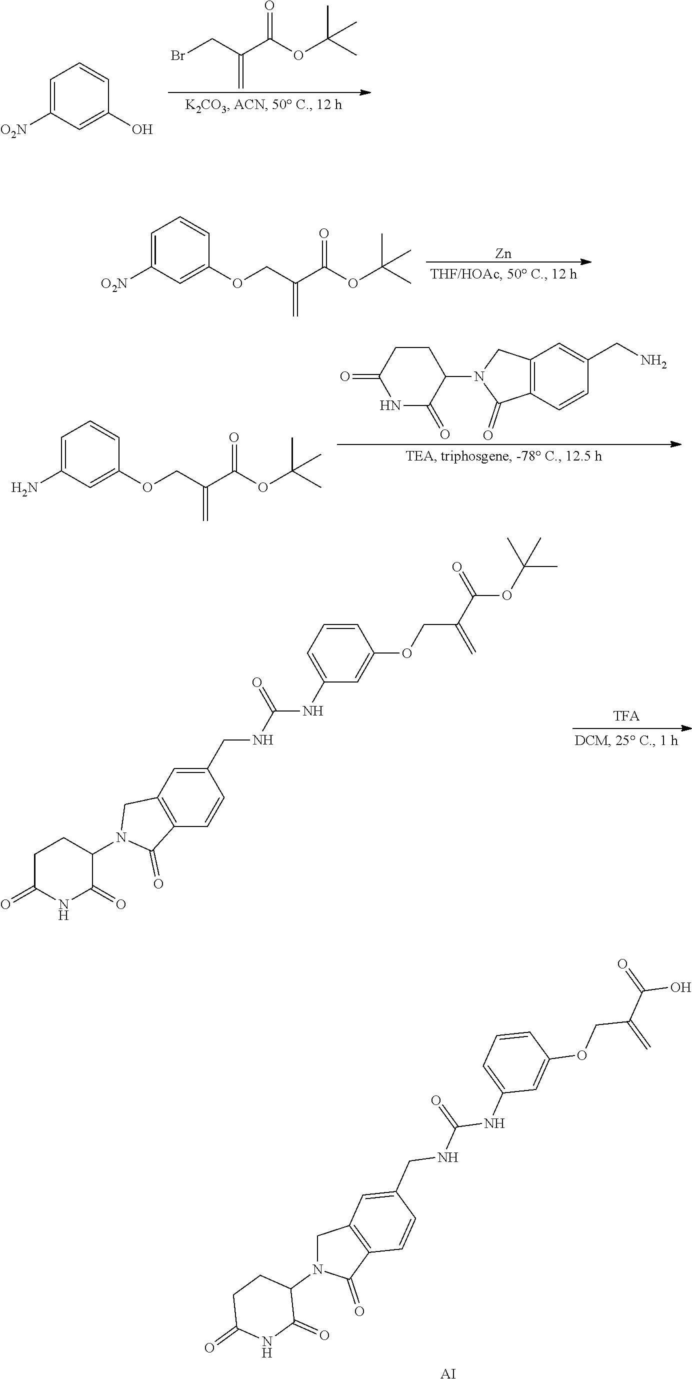

Step 1. To a solution of 3-nitrophenol (2.0 g, 14.4 mmol, 2.9 mL, 1.0 equiv) and tert-butyl 2-(bromomethyl)prop-2-enoate (3.20 g, 14.4 mmol, 1.0 eq) in ACN (15 mL) was added K₂CO₃ (5.90 g, 43.1 mmol, 3.0 eq). The mixture was stirred at 50° C. for 12 h. The reaction mixture was concentrated under reduced pressure to give a residue. The residue was purified by column chromatography (SiO₂, Petroleum ether/Ethyl acetate=10/1 to 5/1). Compound tert-butyl 2-[(3-nitrophenoxy)methyl]prop-2-enoate (1.5 g, 5.4 mmol, 37% yield) was obtained as a colorless oil.

Step 2. To a solution of tert-butyl 2-[(3-nitrophenoxy)methyl]prop-2-enoate (1.5 g, 5.4 mmol, 1.0 eq) in THF (10 mL) and HOAc (5.0 mL) was added Zn (1.50 g, 26.9 mmol, 5.0 eq). The mixture was stirred at 50° C. for 12 h. The reaction mixture was filtered and concentrated under reduced pressure to give a residue. The residue was purified by prep-HPLC (column: Waters Xbridge C18 150×50 mm×10 um; mobile phase: [water(NH₄HCO₃)-ACN]; B %: 36%-66%, 10 min). Compound tert-butyl 2-[(3-aminophenoxy)methyl]prop-2-enoate (500 mg, 2.1 mmol, 37% yield) was obtained as a white solid. ¹H NMR (400 MHz, CDCl₃) δ 7.27 (s, 1H), 7.06 (t, J=7.9 Hz, 2H), 6.38-6.26 (m, 8H), 5.90 (s, 2H), 4.69 (s, 4H), 4.17-4.08 (m, 1H), 3.66 (m, 4H), 2.05 (s, 1H), 1.61-1.55 (m, 1H), 1.50-1.43 (m, 1H), 1.31-1.22 (m, 2H).

Step 3. To a solution of triphosgene (223 mg, 750 μmol, 0.8 eq) in THF (10 mL) was added TEA (1.0 g, 10.0 mmol, 1.4 mL, 10.0 eq) and tert-butyl 2-[(3-aminophenoxy)methyl]prop-2-enoate (250 mg, 1.0 mmol, 1.0 eq) in THF (5.0 mL) at −78° C. The mixture was stirred at −78° C. for 0.5 h and 3-[5-(aminomethyl)-1-oxo-isoindolin-2-yl]piperidine-2,6-dione (310 mg, 1.0 mmol, 1.0 eq, HCl salt) was added. The reaction mixture was stirred at 25° C. for 12 h. The reaction mixture was partitioned between 50 mL water and 50 mL ethyl acetate. The organic phase was separated, washed with brine (30 mL×3), dried over anhydrous Na₂SO₄, filtered and concentrated under reduced pressure to give a residue. The residue was purified by prep-TLC (SiO₂, Petroleum ether: Ethyl acetate=0:1). Compound tert-butyl 2-[[3-[[2-(2,6-dioxo-3-piperidyl)-1-oxo-isoindolin-5-yl]methylcarbamoylamino]phenoxy]methyl]prop-2-enoate (80 mg, 0.15 mmol, 15% yield) was obtained as a white solid. ¹H NMR: (400 MHz, CD₃OD) δ 7.77 (d, J=7.8 Hz, 1H), 7.55 (s, 1H), 7.49 (d, J=7.9 Hz, 1H), 7.24-7.07 (m, 2H), 6.95-6.85 (m, 1H), 6.63-6.54 (m, 1H), 6.24 (d, J=1.1 Hz, 1H), 5.89 (d, J=1.4 Hz, 1H), 5.18-5.11 (m, 1H), 4.68 (s, 2H), 4.59-4.40 (m, 4H), 4.16-4.04 (m, 1H), 2.97-2.83 (m, 1H), 2.82-2.73 (m, 1H), 2.56-2.41 (m, 1H), 2.21-2.12 (m, 1H), 2.02 (s, 1H), 1.51 (s, 9H), 1.32-1.13 (m, 2H).

Step 4. To a solution of tert-butyl 2-[[3-[[2-(2,6-dioxo-3-piperidyl)-1-oxo-isoindolin-5-yl]methylcarbamoylamino]phenoxy]methyl]prop-2-enoate (80 mg, 0.15 mmol, 1.0 eq) in DCM (1 mL) was added TFA (0.5 mL) and the mixture was stirred at 25° C. for 0.5 h. The reaction mixture was concentrated under reduced pressure to give a residue. The crude product was used into the next step without further purification. Compound 2-[[3-[[2-(2,6-dioxo-3-piperidyl)-1-oxo-isoindolin-5-yl]methylcarbamoylamino]phenoxy]methyl]prop-2-enoic acid AI (88 mg, 0.15 mmol, TFA salt) was obtained as a yellow oil.

### Compound AJ: 2-((3-(3-((2-(2,6-dioxopiperidin-3-yl)-1-oxoisoindolin-5-yl)methyl)ureido)-5-methoxyphenoxy)methyl)acrylic acid

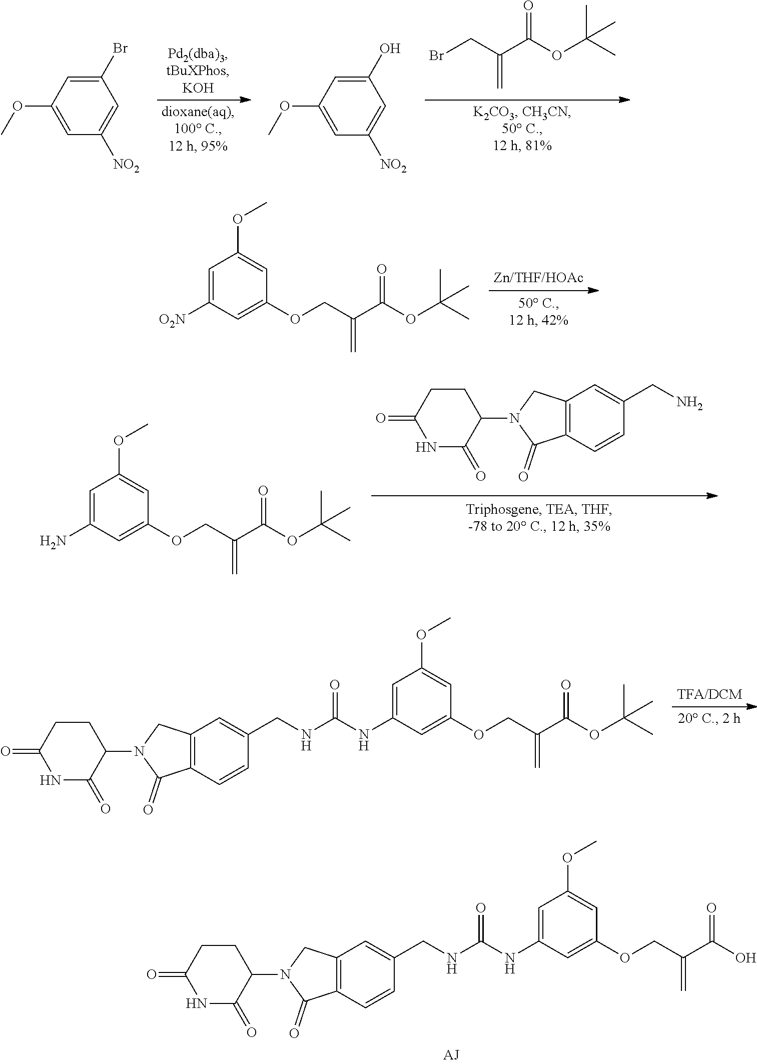

Step 1. To a solution 1-bromo-3-methoxy-5-nitrobenzene (4.00 g, 17.2 mmol, 1.0 eq) in dioxane (26 mL) and H₂O (26 mL) was added KOH (3.87 g, 68.9 mmol, 4.0 eq), Pd₂(dba)₃ (789.3 mg, 861.9 μmol, 0.05 eq), tBuXPhos (1.37 g, 1.72 mmol, 0.1 eq). The suspension was degassed and purged with N₂ for 3 times. The mixture was stirred under N₂ at 100° C. for 12 h. The reaction mixture was filtered. pH was adjusted to 6-7 by HCl (1N). After extracted with EA (3×20 mL), the reaction mixture was dried over anhydrous Na₂SO₄, filtered and concentrated under reduced pressure to give a residue. The residue was purified by column chromatography (SiO₂, Petroleum ether/Ethyl acetate=1/0 to 5/1) to give 3-methoxy-5-nitrophenol (3.10 g, 16.5 mmol, 95% yield) as a yellow solid. ¹H NMR: (400 MHz, CDCl₃) 10.43 (s, 1H), 7.18 (m, J=1.9 Hz, 2H), 6.75 (t, J=2.2 Hz, 1H), 3.81 (s, 3H).

Step 2. To a solution of 3-methoxy-5-nitrophenol (3.79 g, 17.2 mmol, 1.0 eq tert-butyl 2-(bromomethyl)acrylate (2.90 g, 17.2 mmol, 1.0 eq) in CH₃CN (30 mL) was added K₂CO₃ (7.11 g, 51.4 mmol, 3.0 eq). The mixture was stirred at 50° C. for 12 h. The reaction mixture was filtered and concentrated under reduced pressure to give a residue. The residue was purified by column chromatography (SiO₂, Petroleum ether/Ethyl acetate=1/0 to 40/1) to afford tert-butyl 2-((3-methoxy-5-nitrophenoxy)methyl)acrylate (4.30 g, 13.9 mmol, 81% yield) as a yellow solid. ¹H NMR: (400 MHz, CDCl₃) 7.42-7.27 (m,, 2H), 6.79 (t, J=2.3 Hz, 1H), 6.34 (s, 1H), 5.91 (d, J=0.9 Hz, 1H), 4.77 (s, 2H), 3.87 (s, 3H), 1.53 (s, 9H).

Step 3. To a solution of tert-butyl 2-((3-methoxy-5-nitrophenoxy)methyl)acrylate (2.30 g, 7.44 mmol, 1.0 eq) in THF (20 mL), CH₃COOH (10 mL) was added Zn(2.43 g, 43.5 mmol, 5.9 eq). The mixture was stirred at 50° C. for 12 h. The reaction mixture was filtered and concentrated under reduced pressure to give a residue. The residue was purified by prep-HPLC (column: Waters Xbridge C18 150×50 mm×10 um; mobile phase: [water (NH₄HCO₃)-ACN]; B %: 35% to 65%, 10 min) to afford tert-butyl 2-((3-amino-5-methoxyphenoxy)methyl)acrylate (870 mg, 3.11 mmol, 42% yield) as a white solid. ¹H NMR: (400 MHz, CDCl₃) 6.31-6.26 (m, 1H), 5.98-5.95 (m, 1H), 5.93-5.87 (m, 3H), 4.66 (s, 2H), 3.75 (s, 3H), 3.66 (s, 2H), 1.52 (s, 9H).

Step 4. To a solution of triphosgene (740 mg, 2.49 mmol, 2.3 eq) and TEA (1.09 g, 10.7 mmol, 1.49 mL, 10.0 eq) in THF (50 mL) was added tert-butyl 2-((3-amino-5-methoxyphenoxy)methyl)acrylate (300 mg, 1.07 mmol, 1.0 eq) in THF (15 mL) slowly at−78° C., and then it was stirred for 0.5 h. 3-(5-(Aminomethyl)-1-oxoisoindolin-2-yl)piperidine-2,6-dione (332 mg, 1.07 mmol, 1.0 eq, HCl salt) was added to the mixture and then it was stirred at 20° C. for 11.5 h. The reaction mixture was diluted with H₂O (25 mL) and extracted with EtOAc (25 mL×2). The combined organic layers were dried over Na₂SO₄, filtered and concentrated under reduced pressure to give a residue. The residue was purified by column chromatography (SiO₂, Petroleum ether/Ethyl acetate=1/1 to ethyl acetate: methanol=10/1) to tert-butyl 2-((3-(3-((2-(2,6-dioxopiperidin-3-yl)-1-oxoisoindolin-5-yl)methyl)ureido)-5-methoxyphenoxy)methyl)acrylate (220 mg, 380 μmol, 35% yield) as a white solid. ¹H NMR: (400 MHz, CDCl₃) 7.52-7.01 (m, 4H), 6.76-6.53 (m, 2H), 6.34-6.01 (m, 3H), 5.92-5.75 (m, 1H), 5.27-4.76 (m, 1H), 4.70-3.96 (m, 8H), 3.79-3.53 (m, 3H), 2.92-2.51 (m, 2H), 2.37-1.75 (m, 5H), 1.42-1.11 (m, 4H), 0.15-0.02 (m, 3H).

Step 5. To a solution of tert-butyl 2-((3-(3-((2-(2,6-dioxopiperidin-3-yl)-1-oxoisoindolin-5-yl)methyl)ureido)-5-methoxyphenoxy)methyl)acrylate (90.0 mg, 155 μmol, 1.0 eq) in DCM (1 mL) was added TFA (0.5 mL). The mixture was stirred at 20° C. for 2 h. The reaction mixture was concentrated under reduced pressure to give 2-((3-(3-((2-(2,6-dioxopiperidin-3-yl)-1-oxoisoindolin-5-yl)methyl)ureido)-5-methoxyphenoxy)methyl)acrylic acid AJ (99 mg, TFA salt) a brown oil.

### Compound AK: 5-chloro-6-methyl-4-(5-methyl-3-(1-methyl-1H-indazol-5-yl)-1-(2-azaspiro[3.3]heptan-6-yl)-1H-pyrazol-4-yl)-1H-indazole

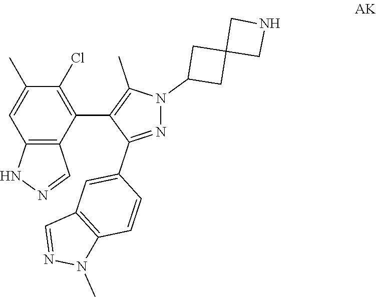 [SMILES: Cc1cc2[nH]ncc2c(-c2c(-c3ccc4c(cnn4C)c3)nn(C3CC4(CNC4)C3)c2C)c1Cl]

Compound AK was prepared based on procedures in WO 2021/124222.

### Compound AL: 2-(Azetidin-3-yl)-5-(3-hydroxynaphthalen-1-yl)-1-methyl-1,2-dihydro-3H-indazol-3-one

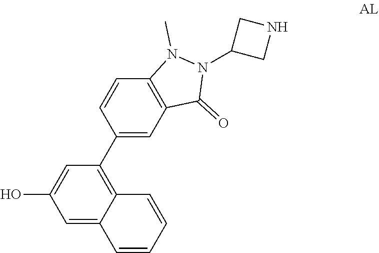 [SMILES: Cn1c2ccc(-c3cc(O)cc4ccccc34)cc2c(=O)n1C1CNC1]

Compound AL was prepared based on procedures in WO 2018/68017.

### Compound AM: 2-(4-(4-((8-(3-Aminophenyl)quinazolin-2-yl)amino)-2,3-difluorophenyl)piperazin-1-yl)ethan-1-ol

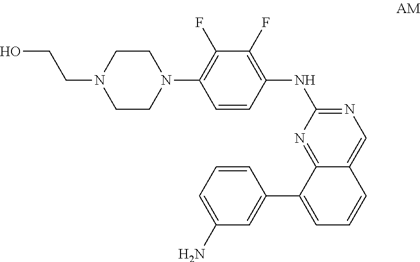 [SMILES: Nc1cccc(-c2cccc3cnc(Nc4ccc(N5CCN(CCO)CC5)c(F)c4F)nc23)c1]

Step 1. To a solution of 1,2,3-trifluoro-4-nitrobenzene (5.00 g, 28.2 mmol, 3.25 mL, 1.0 eq), 2-(piperazin-1-yl)ethan-1-ol (3.68 g, 28.2 mmol, 3.47 mL, 1.0 eq) in DMF (40 mL) was added K₂CO₃ (7.80 g, 56.5 mmol, 2.0 eq) at 0° C. The mixture was stirred at 20° C. for 12 h. This reaction mixture was poured into 200 mL ice-water. The resulting solid was collected by filtration, washed with cold water three times, and concentrated under reduce pressure to afford 2-(4-(2,3-difluoro-4-nitrophenyl)piperazin-1-yl)ethan-1-ol (6.10 g, 21.2 mmol, 75% yield) as a yellow solid. ¹H NMR: (400 MHz, CDCl₃) δ 7.89-7.80 (m, 1H), 6.64-6.69 (m, J=1.9, 7.9, 9.5 Hz, 1H), 3.67 (s, 2H), 3.41-3.34 (m, 4H), 2.72-2.67 (m, 4H), 2.65-2.61 (m, 2H).

Step 2. To a solution of 2-(4-(2,3-difluoro-4-nitrophenyl)piperazin-1-yl)ethan-1-ol (6.10 g, 21.2 mmol, 1.0 eq) in MeOH (60 mL) was added Pd/C (610 mg, 21.2 mmol, 10% purity, 1.0 eq) under N₂ atmosphere. The suspension was degassed and purged with H₂ for 3 times. The mixture was stirred under H₂ (15 psi) at 20° C. for 12 h. The reaction mixture was filtered and concentrated under reduced pressure to afford 2-(4-(4-amino-2,3-difluorophenyl)piperazin-1-yl)ethan-1-ol (5.4 g) as a brown oil and used for the next step directly. ¹H NMR: (400 MHz, CDCl₃) δ 6.61-6.54 (m, 1H), 6.52-6.43 (m, 1H), 3.69-3.48 (m, 5H), 3.08-2.95 (m, 5H), 2.75-2.67 (m, 4H), 2.62 (t, J=5.4 Hz, 2H).

Step 3. To a solution of 8-bromo-2-chloroquinazoline (7.11 g, 29.2 mmol, 1.0 eq) and (3-aminophenyl)boronic acid (4.00 g, 29.2 mmol, 1.0 eq) in H₂O (8 mL) and dioxane (80 mL) was added Pd(dppf)Cl₂ (3.21 g, 4.38 mmol, 0.15 eq) and Na₂CO₃ (6.19 g, 58.42 mmol, 2.0 eq). The mixture was stirred at 80° C. for 12 h under N₂. The reaction mixture was filtered and concentrated under reduced pressure to give a residue. The residue was purified by column chromatography (SiO₂, Petroleum ether/Ethyl acetate=5/1 to 1/1) to give 2-(4-(4-((8-(3-aminophenyl)quinazolin-2-yl)amino)-2,3-difluorophenyl)piperazin-1-yl)ethan-1-ol (4.30 g, 10.9 mmol, 37% yield) as a yellow solid.

Step 4. To a solution of 2-(4-(4-((8-(3-aminophenyl)quinazolin-2-yl)amino)-2,3-difluorophenyl)piperazin-1-yl)ethan-1-ol (5.50 g, 21.5 mmol, 1.0 eq) in DCM (50 mL) was added TEA (6.53 g, 64.5 mmol, 8.98 mL, 3.0 eq) and TFAA (6.78 g, 32.3 mmol, 4.49 mL, 1.5 eq). The mixture was stirred at 20° C. for 12 h. The reaction mixture was concentrated under reduced pressure to give a residue. The residue was purified by column chromatography (SiO₂, Petroleum ether/Ethyl acetate=7/1 to 0/1) to afford N-(3-(2-chloroquinazolin-8-yl)phenyl)-2,2,2-trifluoroacetamide (2.85 g, 8.10 mmol, 38% yield) as a yellow solid. ¹H NMR: (400 MHz, CDCl₃) δ 9.36 (s, 1H), 8.47-8.29 (m, 1H), 8.05-7.85 (m, 3H), 7.80-7.73 (m, 1H), 7.73-7.64 (m, 1H), 7.59-7.48 (m, 2H), 5.34 (d, J=12.4 Hz, 1H), 3.45-3.28 (m, 3H).

Step 5. To a solution of N-(3-(2-chloroquinazolin-8-yl)phenyl)-2,2,2-trifluoroacetamide (1.00 g, 2.84 mmol, 1.0 eq), 2-(4-(4-amino-2,3-difluorophenyl)piperazin-1-yl)ethan-1-ol (731 mg, 2.84 mmol, 1.0 eq) in 2-methoxyethanol (10 mL) was added HCl (12 M, 24 μL, 0.1 eq). The mixture was stirred at 100° C. for 1 h. The reaction mixture was filtered and concentrated under reduced pressure to give a residue. The residue was purified by prep-HPLC (column: Phenomenex luna C18 150×40 mm×15 um; mobile phase: [water (FA)-ACN]; B %: 20%-50%, 10 min) to afford N-(3-(2-((2,3-difluoro-4-(4-(2-hydroxyethyl)piperazin-1-yl)phenyl)amino)quinazolin-8-yl)phenyl)-2,2,2-trifluoroacetamide (300 mg, 524 μmol, 18% yield) as a yellow solid. ¹H NMR: (400 MHz, CD₃OD): δ 9.17-9.13 (m, 1H), 8.57-8.46 (m, 1H), 8.22-8.15 (m, 1H), 8.00 (s, 1H), 7.78-7.85 (m, J=1.3, 7.6, 18.3 Hz, 2H), 7.71 (d, J=2.2, 6.8 Hz, 1H), 7.59-7.53 (m, 2H), 7.49-7.39 (m, 2H), 6.50 (t, J=1.9, 8.8 Hz, 1H), 3.92-3.88 (m, 2H), 3.32-3.25 (m, 4H), 3.16-3.07 (m, 4H), 3.01-2.94 (m, 2H).

Step 6. To a solution of N-(3-(2-((2,3-difluoro-4-(4-(2-hydroxyethyl)piperazin-1-yl)phenyl)amino)quinazolin-8-yl)phenyl)-2,2,2-trifluoroacetamide (200 mg, 349 μmol, 1.0 eq) in THF (1 mL) was added LiOH·H₂O (36.6 mg, 873 μmol, 2.5 eq) in H₂O (1 mL). The mixture was stirred at 20° C. for 12 h. The reaction mixture was diluted with 15 mL H₂O and extracted with EtOAc (15 mL×2). The combined organic layers were dried over Na₂SO₄, filtered and concentrated under reduced pressure to give 2-(4-(4-((8-(3-aminophenyl)quinazolin-2-yl)amino)-2,3-difluorophenyl)piperazin-1-yl)ethan-1-ol AM (145 mg, 304 μmol, 87% yield) as a yellow solid.

### Compound AN: 2-((3-chloro-5-(3-((2-(2,6-dioxopiperidin-3-yl)-6-fluoro-1-oxoisoindolin-5-yl)methyl)ureido)phenoxy)methyl)acrylic acid

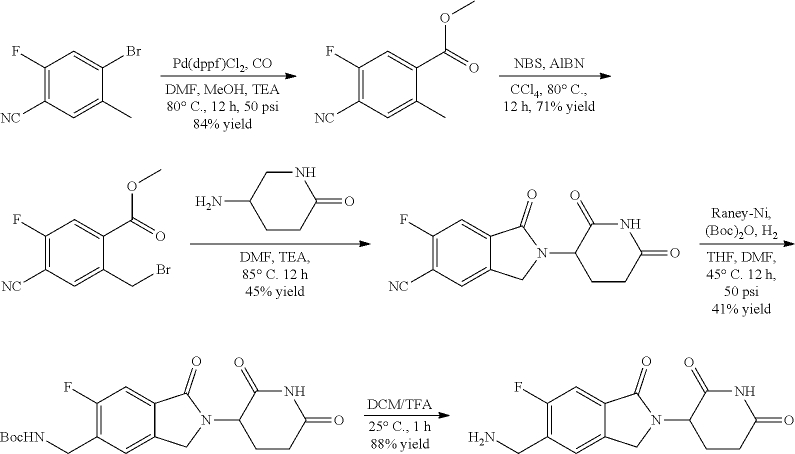

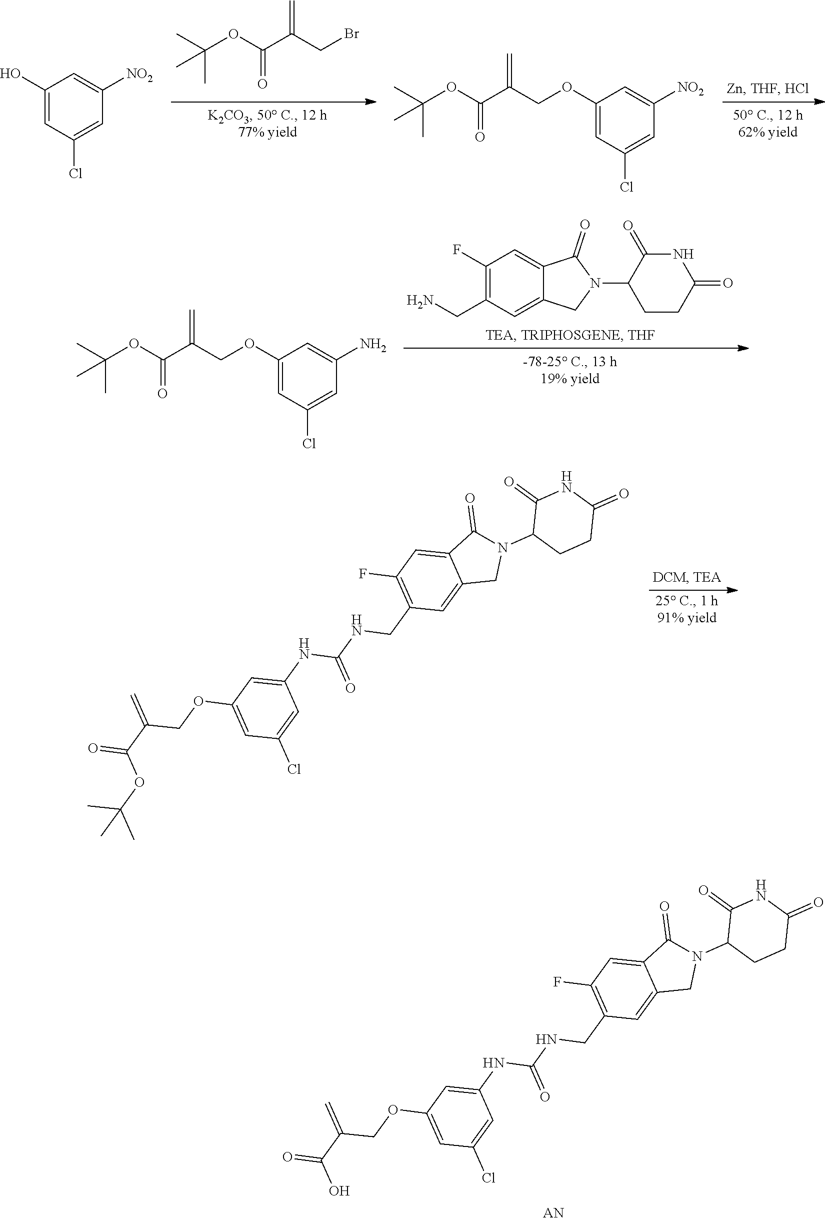

Step 1. A mixture of 4-bromo-2-fluoro-5-methyl-benzonitrile (5.0 g, 23.3 mmol, 1.0 eq), Pd(dppf)Cl₂ (1.7 g, 2.4 mmol, 0.1 eq), TEA (11.8 g, 116.8 mmol, 16.2 mL, 5.0 eq) in DMF (40 mL) and MeOH (20 mL) was degassed and purged with CO for 3 times, and then the mixture was stirred at 80° C. for 12 h. The mixture filtered and concentrated to give the residue. The residue was purified by the column chromatography (SiO₂, Petroleum ether/Ethyl acetate=20/1 to 10/1) to give the 4-bromo-2-fluoro-5-methylbenzonitrile (4.2 g, 21.7 mmol, 84% yield) as a white solid. ¹H NMR: (400 MHz, CDCl₃) δ=7.74 (d, J=9.2 Hz, 1H), 7.52 (d, J=6.0 Hz, 1H), 3.94 (s, 3H), 2.59 (s, 3H).

Step 2. To a solution of methyl 4-cyano-5-fluoro-2-methyl-benzoate (2.0 g, 10.3 mmol, 1.0 equiv) in CCl₄ (80 mL) was added NBS (2.0 g, 11.4 mmol, 1.1 equiv) and AIBN (850 mg, 5.2 mmol, 0.5 equiv). The mixture was stirred at 80° C. for 12 h. The mixture was filtered and concentrated in vacuum and then the reaction mixture was added 30 mL water, the aqueous phase was extracted with DCM (30 mL×3). The combined organic phase was washed with brine (30 mL×3), dried with anhydrous sodium sulfate, filtered and concentrated in vacuum to give a residue. The residue was purified by column chromatography (SiO₂, Petroleum ether/Ethyl acetate=10/1) to give the methyl 2-(bromomethyl)-4-cyano-5-fluoro-benzoate (2 g, 7.3 mmol, 71% yield) as a colorless oil. ¹H NMR: (400 MHz, CDCl₃) δ=7.85-7.70 (m, 2H), 4.89 (s, 2H), 3.99 (s, 3H).

Step 3. To a solution of methyl 2-(bromomethyl)-4-cyano-5-fluoro-benzoate (2.0 g, 7.4 mmol, 1.0 eq) in DMF (20 mL) was added 3-aminopiperidine-2,6-dione (1.1 g, 8.8 mmol, 1.2 eq) and then added TEA (2.2 g, 22.0 mmol, 3.1 mL, 3.0 eq). The mixture was stirred at 85° C. for 12 h. The reaction mixture was concentrated under reduced pressure to give a residue. The residue was purified by prep-HPLC (column: Phenomenex luna C18 (250×70 mm, 15 m); mobile phase: [water(NH₄HCO₃)-ACN]; B %: 15%-45%, 20 min) to give the 2-(2,6-dioxo-3-piperidyl)-6-fluoro-1-oxo-isoindoline-5-carbonitrile (960 mg, 3.3 mmol, 45% yield) as a black solid. ¹H NMR: (400 MHz, DMSO-d6) δ=11.04 (s, 1H), 8.25 (d, J=5.6 Hz, 1H), 7.89 (d, J=8.0 Hz, 1H), 5.22-5.12 (m, 1H), 4.59-4.20 (m, 2H), 2.97-2.82 (m, 1H), 2.65-2.54 (m, 1H), 2.46-2.40 (m, 1H), 2.07-2.00 (m, 1H).

Step 4. To a solution of 2-(2,6-dioxo-3-piperidyl)-6-fluoro-1-oxo-isoindoline-5-carbonitrile (960 mg, 3.3 mmol, 1.0 eq) in THF (20 mL) and DMF (10 mL) was added Raney-Ni (1.0 g, 11.7 mmol, 3.5 eq) and (Boc)₂0 (2.2 g, 10.0 mmol, 2.3 mL, 3.0 eq). The mixture was degassed with H₂ three times and stirred at 45° C. for 12 h. The mixture was cooled to 25° C. and filtered. The filtrate was concentrated to give the residue. The residue was purified by prep-HPLC (column: mobile phase: [water(TFA)-ACN]; B %: 15%-45%, 20 min) to give the tert-butyl N-[[2-(2,6-dioxo-3-piperidyl)-6-fluoro-1-oxo-isoindolin-5-yl]methyl]carbamate (540 mg, 1.4 mmol, 41% yield) as a white solid. ¹H NMR: (400 MHz, CDCl₃) δ=7.94 (s, 1H), 7.64-7.44 (m, 2H), 5.29-5.15 (m, 1H), 5.05 (d, J=2.4 Hz, 1H), 4.51-4.34 (m, 4H), 3.01-2.67 (m, 2H), 2.46-2.31 (m, 1H), 2.29-2.20 (m, 1H), 1.47 (s, 9H).

Step 5. To a solution of tert-butyl N-[[2-(2,6-dioxo-3-piperidyl)-6-fluoro-1-oxo-isoindolin-5-yl]methyl]carbamate (540 mg, 1.4 mmol, 1.0 eq) in DCM (10 mL) was added HCl/dioxane (10 mL). The mixture was stirred at 25° C. for 1 h. The reaction mixture was concentrated under reduced pressure to give 3-[5-(aminomethyl)-6-fluoro-1-oxo-isoindolin-2-yl]piperidine-2,6-dione (400 mg, 1.2 mmol, 88% yield, HCl) as a white solid.

Step 6. To a solution of 3-chloro-5-nitro-phenol (3.0 g, 17.3 mmol, 1.0 eq) and tert-butyl 2-(bromomethyl)prop-2-enoate (4.6 g, 20 mmol, 1.2 eq) in MeCN (30 mL) was added K₂CO₃ (7.2 g, 51.8 mmol, 3.0 eq). The mixture was stirred at 50° C. for 12 h. The reaction mixture was filtered and the filtrate was concentrated under reduced pressure to give a residue. The residue was purified by column chromatography (SiO₂, Petroleum ether: Ethyl acetate=5:1) to give the tert-butyl 2-[(3-chloro-5-nitro-phenoxy)methyl]prop-2-enoate (4.2 g, 13.4 mmol, 77% yield) as a yellow oil. ¹H NMR: (400 MHz, CDCl₃) δ=7.84 (s, 1H), 7.83 (s, 1H), 7.82 (s, 2H), 6.45 (s, 1H), 5.95 (s, 2H), 1.52 (s, 9H).

Step 7. To a solution of tert-butyl 2-[(3-chloro-5-nitro-phenoxy) methyl]prop-2-enoate (4 g, 12.7 mmol, 1.0 eq) in THF (40 mL) and HCl (20 mL) was added Zn (4.5 g, 69.6 mmol, 5.0 eq). The mixture was stirred at 50° C. for 12 h. The mixture was added 60 mL NaHCO₃, the aqueous phase was extracted with DCM (30 mL×3). The combined organic phase was washed with brine (30 mL×3), dried over anhydrous sodium sulfate, filtered and concentrated under vacuum to give a residue. The residue was purified by column chromatography (SiO₂, Petroleum ether/Ethyl acetate=20/1 to 10/1) to give the tert-butyl 2-[(3-amino-5-chloro-phenoxy)methyl]prop-2-enoate (2.3 g, 7.9 mmol, 62% yield) as a yellow oil. ¹H NMR: (400 MHz, CDCl₃) δ=6.52-6.19 (m, 4H), 5.87 (d, J=1.2 Hz, 1H), 5.04 (s, 2H), 4.65 (s, 2H), 1.52 (s, 9H).

Step 8. To a solution of triphosgene (80 mg, 269 μmol, 0.5 eq) in THF (5 mL) was added TEA (267 mg, 2.6 mmol, 367 μL, 5.0 eq) and tert-butyl 2-[(3-amino-5-chloro-phenoxy)methyl]prop-2-enoate (150 mg, 528 μmol, 1.0 eq) in THF (5 mL). The mixture was stirred at −78° C. for 1 h. Then 3-[5-(aminomethyl)-6-fluoro-1-oxo-isoindolin-2-yl]piperidine-2,6-dione (153 mg, 528 μmol, 1.0 eq) was added to the mixture. The mixture was stirred at 25° C. for 12 h. The mixture was diluted with water (20 mL) and saturated NaHCO₃ (10 mL). The aqueous phase was extracted with ethyl acetate (20 mL×2). The combined organic phase was washed with brine (20 mL×2), dried over anhydrous sodium sulfate, filtered and concentrated under vacuum to give a residue. The residue was purified by prep-HPLC (column: Phenomenex luna C18 150×25 mm×10 um; mobile phase: [water(FA)-ACN]; B %: 43%-73%, 15 min) to give the tert-butyl 2-[[3-chloro-5-[[2-(2,6-dioxo-3-piperidyl)-6-fluoro-1-oxo-isoindolin-5-yl]methylcarbamoylamino]phenoxy]methyl]prop-2-enoate (60 mg, 99 μmol, 19% yield) as a white solid. ¹H NMR: (400 MHz, CDCl₃) δ=8.44 (s, 1H), 7.56-7.42 (m, 2H), 7.18 (d, J=8.4 Hz, 1H), 7.04 (s, 1H), 6.91 (s, 1H), 6.57 (s, 1H), 6.28 (d, J=1.2 Hz, 1H), 5.92-5.81 (m, 2H), 5.21-5.03 (m, 1H), 4.64 (s, 2H), 4.54-4.41 (m, 2H), 4.29 (d, J=7.2 Hz, 2H), 3.00-2.70 (m, 2H), 2.46-2.16 (m, 2H), 1.51 (s, 9H).

Step 9. A solution of tert-butyl 2-[[3-chloro-5-[[2-(2,6-dioxo-3-piperidyl)-6-fluoro-1-oxo-isoindolin-5-yl]methylcarbamoylamino]phenoxy]methyl]prop-2-enoate (60 mg, 99 μmol, 1.0 eq) in DCM (3 mL) and TFA (1 mL) was stirred at 25° C. for 1 h. The mixture was concentrated under reduced pressure to give a residue. The residue 2-[[3-chloro-5-[[2-(2,6-dioxo-3-piperidyl)-6-fluoro-1-oxo-isoindolin-5-yl]methylcarbamoylamino]phenoxy]methyl]prop-2-enoic acid (60 mg, 91 μmol, 91% yield, TFA) as a white solid was used for next step without further purification.

### Compound AO: 2-((3-(3-((2-(2,6-dioxopiperidin-3-yl)-4-fluoro-1-oxoisoindolin-5-yl)methyl)ureido)-5-(trifluoromethyl)phenoxy)methyl)acrylic acid

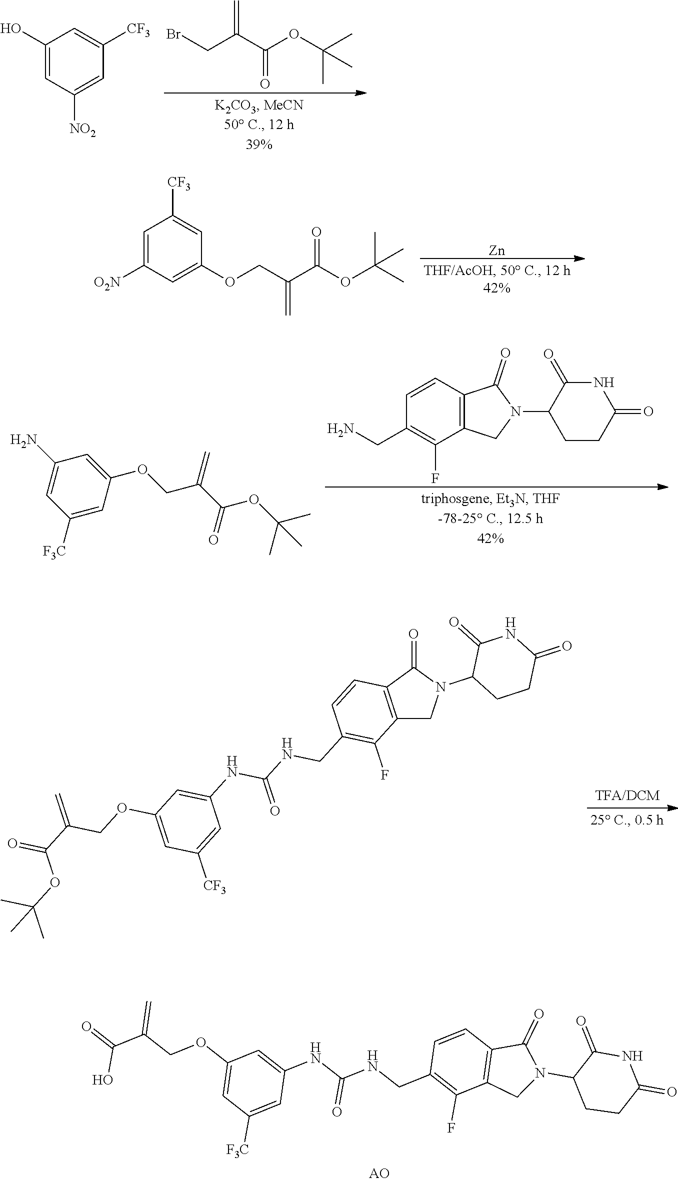

Step 1. To a solution of 3-nitro-5-(trifluoromethyl)phenol (2.00 g, 9.66 mmol, 1.0 eq) and tert-butyl 2-(bromomethyl)prop-2-enoate (2.14 g, 9.66 mmol, 1.0 eq) in ACN (15 mL) was added K₂CO₃ (4.00 g, 29.0 mmol, 3.0 eq). The mixture was stirred at 50° C. for 12 h. The residue was purified by column chromatography (SiO₂, Petroleum ether/Ethyl acetate=10/1 to 5/1) to give compound tert-butyl 2-[[3-nitro-5-(trifluoromethyl) phenoxy] methyl]prop-2-enoate (1.3 g, 39% yield) as a white solid. ¹H NMR (400 MHz, CDCl₃): δ 8.25 (s, 2H), 8.01 (s, 1H), 7.50 (s, 1H), 6.40 (s, 1H), 6.01 (s, 1H), 4.70 (s, 2H), 1.5 (s, 9H).

Step 2. To a solution of tert-butyl 2-[[3-nitro-5-(trifluoromethyl) phenoxy] methyl]prop-2-enoate (1.30 g, 3.74 mmol, 1.0 eq) in THF (10 mL) and HOAc (5 mL) was added Fe (1.05 g, 18.7 mmol, 5.0 eq). The mixture was stirred at 50° C. for 12 h. The reaction mixture was filtered and concentrated under reduced pressure to give a residue. The residue was purified by prep-HPLC (column: Waters Xbridge C18 150×50 mm×10 um; mobile phase: [water (NH₄HCO₃)-ACN]; B %: 44%-74%, 10 min) to give compound tert-butyl 2-[[3-amino-5-(trifluoromethyl)phenoxy]methyl]prop-2-enoate (500 mg, 42% yield) as a white solid.

Step 3. To a solution of triphosgene (168 mg, 567 μmol, 0.75 eq) in THF (10 mL) was added tert-butyl 2-[[3-amino-5-(trifluoromethyl)phenoxy]methyl]prop-2-enoate (240 mg, 756 μmol, 1.0 eq) and Et₃N (765 mg, 7.56 mmol, 1.05 mL, 10 eq) in THF (2 mL). The mixture was stirred at −78° C. for 0.5 h. Then 3-[5-(aminomethyl)-4-fluoro-1-oxo-isoindolin-2-yl]piperidine-2,6-dione (230 mg, 567 μmol, 0.75 eq, TFA salt) was added. The mixture was stirred at −78° C. for 0.5 h. The mixture was stirred at 25° C. for 0.5 h. The mixture was added 50 mL H₂O and extracted with EA (50 mL×3). The combined organic layers were filtered and concentrated under reduced pressure to give a residue. The residue was purified by prep-TLC (SiO₂, DCM: MeOH=10:1) to give compound tert-butyl 2-[[3-[[2-(2,6-dioxo-3-piperidyl)-4-fluoro-1-oxo-isoindolin-5-yl] methylcarbamoylamino]-5-(trifluoromethyl) phenoxy] methyl]prop-2-enoate (0.2 g, 42% yield) as a white solid.

Step 4. To a solution of tert-butyl 2-[[3-[[2-(2,6-dioxo-3-piperidyl)-4-fluoro-1-oxo-isoindolin-5-yl] methylcarbamoylamino]-5-(trifluoromethyl) phenoxy] methyl]prop-2-enoate (100 mg, 158 μmol, 1.0 eq) in DCM/TFA=1:1 (158 μmol, 1 mL) was stirred at 25° C. for 0.5 h. The reaction mixture was concentrated under reduced pressure to remove solvent to give compound 2-[[3-[[2-(2,6-dioxo-3-piperidyl)-4-fluoro-1-oxo-isoindolin-5-yl]methylcarbamoyl-amino]-5-(trifluoromethyl)phenoxy]methyl]prop-2-enoic acid AO (91 mg, 99% yield) as a colorless oil.

### Compound AP: 2-((2-chloro-6-(3-((2-(2,6-dioxopiperidin-3-yl)-1-oxoisoindolin-5-yl)methyl)ureido)phenoxy)methyl)acrylic acid

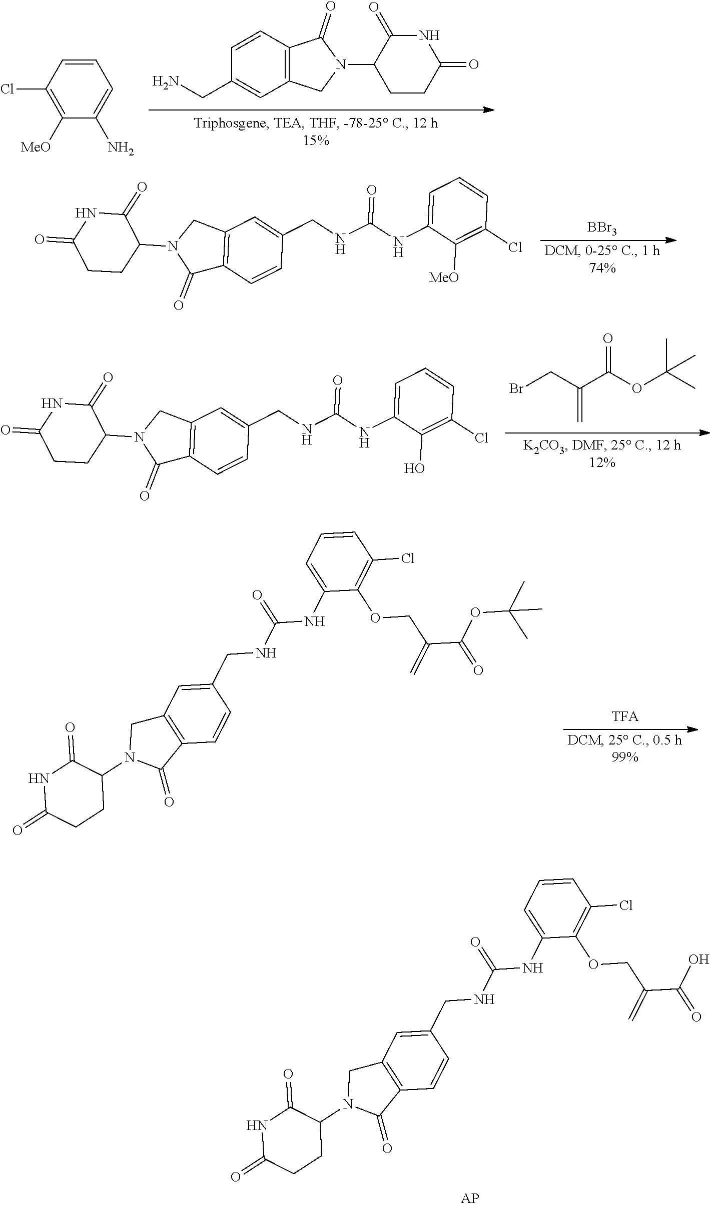

Step 1. To a solution of triphosgene (1.62 g, 5.45 mmol, 0.8 eq) in THF (60 mL) was added 3-chloro-2-methoxy-aniline (1.00 g, 6.35 mmol, 1.0 eq), TEA (6.42 g, 63.5 mmol, 9 mL, 10.0 eq) in THF (20 mL) slowly at −78° C., and then it was stirred for 0.5 h. 3-chloro-2-methoxy-aniline (1.97 g, 6.35 mmol, 1.0 eq, HCl salt) was added to the mixture and then it was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Phenomenex luna C18 150×40 mm×15 um; mobile phase: [water(FA)-ACN]; B %: 27%-57%, 10 min). Compound 1-(3-chloro-2-methoxy-phenyl)-3-[[2-(2,6-dioxo-3-piperidyl)-1-oxo-isoindolin-5-yl]methyl]urea (440 mg, 953 μmol, 15% yield) was obtained as a white solid.

Step 2. To a solution of 1-(3-chloro-2-methoxy-phenyl)-3-[[2-(2,6-dioxo-3-piperidyl)-1-oxo-isoindolin-5-yl]methyl]urea (440 mg, 963 μmol, 1.0 eq) in DCM (9 mL) was added BBr₃ (1.21 g, 4.82 mmol, 464 μL, 5.0 eq) at 0° C. The mixture was stirred at 0-25° C. for 1 h. The reaction mixture was quenched by addition H₂O (2 mL) at 0° C., and the solution was concentrated. After triturated with water (20 mL), the filter cake was dried in vacuum. Compound 1-(3-chloro-2-hydroxy-phenyl)-3-[[2-(2,6-dioxo-3-piperidyl)-1-oxo-isoindolin-5-yl]methyl]urea (315 mg, 711 μmol, 74% yield) was obtained as a yellow solid. ¹H NMR: (DMSO-d₆, 400 MHz) 10.98 (s, 1H), 10.10-9.73 (m, 1H), 8.50-8.18 (m, 1H), 7.84-7.64 (m, 2H), 7.58-7.48 (m, 2H), 7.48-7.41 (m, 1H), 7.02-6.90 (m, 1H), 6.83-6.64 (m, 1H), 5.16-5.00 (m, 1H), 4.51-4.40 (m, 3H), 4.36-4.23 (m, 1H), 3.00-2.81 (m, 1H), 2.68-2.62 (m, 1H), 2.41-2.31 (m, 1H), 2.07-1.93 (m, 1H).

Step 3. To a solution of 1-(3-chloro-2-hydroxy-phenyl)-3-[[2-(2,6-dioxo-3-piperidyl)-1-oxo-isoindolin-5-yl]methyl]urea (310 mg, 700 μmol, 1.0 eq) and tert-butyl 2-(bromomethyl)prop-2-enoate (155 mg, 700 μmol, 1.0 eq) in DMF (1 mL) was added K₂CO₃ (290 mg, 2.10 mmol, 3.0 eq). The mixture was stirred at 25° C. for 12 h. To the reaction mixture was added water (10 mL) and the mixture was extracted with EtOAc (10 mL). The combined organic phase was washed with brine (10 mL×3), dried over anhydrous Na₂SO₄, filtered and concentrated in vacuum. The residue was purified by prep-HPLC (column: Waters Xbridge C18 150×50 mm×10 um; mobile phase: [water(NH₄HCO₃)-ACN]; B %: 37%-67%, 10 min). The residue was further purified by prep-TLC (SiO₂, DCM: MeOH=10:1). Compound tert-butyl 2-[[2-chloro-6-[[2-(2,6-dioxo-3-piperidyl)-1-oxo-isoindolin-5-yl]methylcarbamoylamino]phenoxy]methyl]prop-2-enoate (60 mg, 82 μmol, 12% yield) was obtained as a yellow oil. ¹H NMR: (DMSO-d₆, 400 MHz) 11.13-10.79 (m, 1H), 8.20-8.16 (m, 1H), 8.12-8.08 (m, 1H), 7.74-7.67 (m, 1H), 7.55-7.52 (m, 1H), 7.48-7.38 (m, 2H), 7.11-6.99 (m, 2H), 6.33-6.28 (m, 1H), 6.09-6.02 (m, 1H), 5.15-5.06 (m, 1H), 4.64-4.57 (m, 2H), 4.44-4.39 (m, 3H), 4.36-4.28 (m, 1H), 1.43-1.36 (m, 12H), 1.25-1.22 (m, 1H).

Step 4. To a solution of tert-butyl 2-[[2-chloro-6-[[2-(2,6-dioxo-3-piperidyl)-1-oxo-isoindolin-5-yl]methylcarbamoylamino]phenoxy]methyl]prop-2-enoate (50 mg, 86 μmol, 1.0 equiv) in DCM (2 mL) was added TFA (1 mL). The mixture was stirred at 25° C. for 0.5 h. The reaction mixture was concentrated to afford crude product. 2-[[2-chloro-6-[[2-(2,6-dioxo-3-piperidyl)-1-oxo-isoindolin-5-yl]methylcarbamoylamino]phenoxy]methyl]prop-2-enoic acid AP (46 mg) was obtained as a colorless oil and it was used into the next step without further purification.

### Compound AQ: 2-((4-chloro-2-(3-((2-(2,6-dioxopiperidin-3-yl)-1-oxoisoindolin-5-yl)methyl)ureido)phenoxy)methyl)acrylic acid

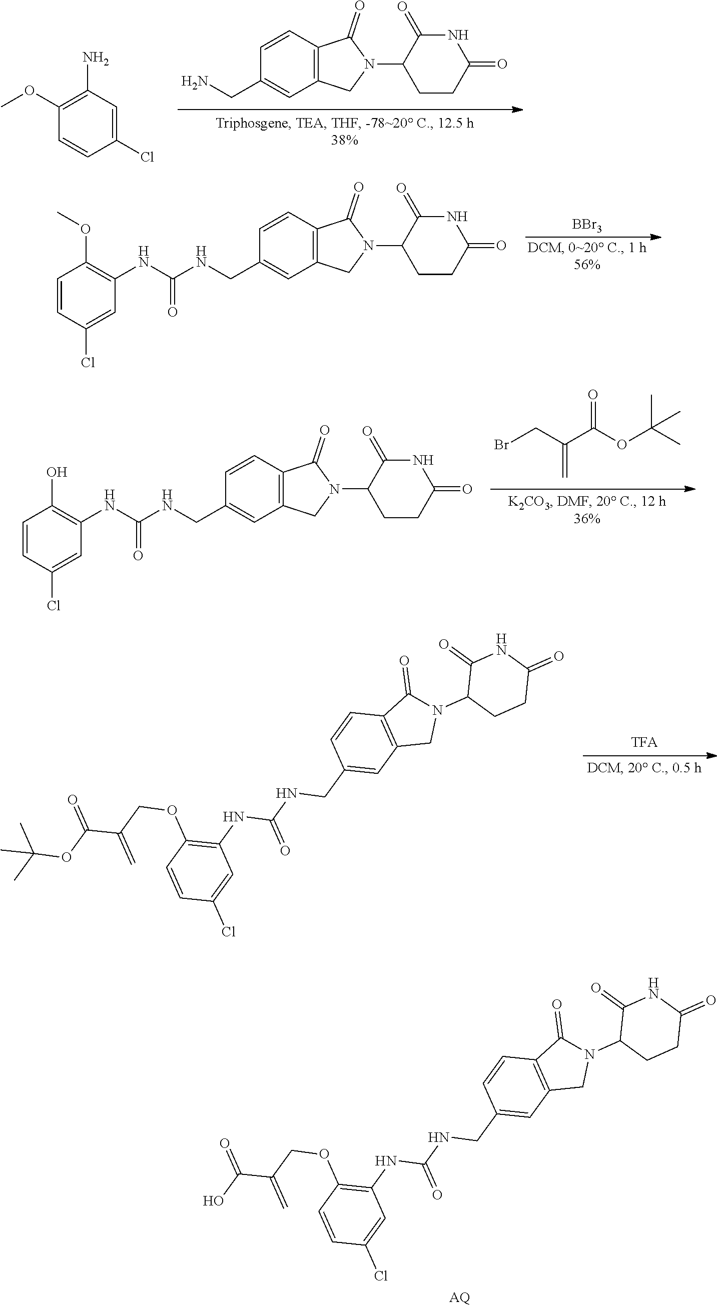

Step 1. To a solution of triphosgene (363 mg, 1.22 mmol, 0.8 eq) in THF (30 mL) was added 5-chloro-2-methoxyaniline (200 mg, 1.27 mmol, 1.0 eq) and TEA (1.28 g, 12.7 mmol, 1.77 mL, 10.0 eq) in THF (10 mL) slowly at −78° C., and then it was stirred for 0.5 h. 3-(5-(Aminomethyl)-1-oxoisoindolin-2-yl)piperidine-2,6-dione (0.393 g, 1.27 mmol, 1.0 eq, HCl salt) was added to the mixture and then it was stirred at 20° C. for 12 h. The reaction mixture was diluted with 30 mL H₂O and extracted with EA (15 mL×2). The combined organic layers were dried over Na₂SO₄, filtered and concentrated under reduced pressure to give a residue. The residue was purified by column chromatography (SiO₂, Petroleum ether/Ethyl acetate=10/1 to 1/1) to afford 1-(5-chloro-2-methoxyphenyl)-3-((2-(2,6-dioxopiperidin-3-yl)-1-oxoisoindolin-5-yl)methyl)urea (220 mg, 481 μmol, 38% yield) as a white solid. ¹H NMR: (400 MHz, CDCl₃): δ 8.23-8.20 (m, 1H), 7.98-7.93 (m, 1H), 7.80-7.75 (m, 1H), 7.51-7.47 (m, 1H), 7.44-7.38 (m, 1H), 7.05-7.00 (m, 1H), 6.93 (d, J=2.5, 8.6 Hz, 1H), 6.75 (d, J=8.8 Hz, 1H), 5.36-5.26 (m, 1H), 5.24-5.17 (m, 1H), 4.58-4.53 (m, 2H), 4.43 (s, 1H), 4.29 (s, 1H), 3.84-3.80 (m, 3H), 3.77-3.70 (m, 2H), 2.96-2.90 (m, 1H), 2.82 (s, 1H), 2.40-2.31 (m, 1H), 2.20-2.25 (m, J=2.2, 5.0, 10.5 Hz, 1H).

Step 2. To a solution of 1-(5-chloro-2-methoxyphenyl)-3-((2-(2,6-dioxopiperidin-3-yl)-1-oxoisoindolin-5-yl)methyl)urea (220 mg, 481 μmol, 1.0 eq) in DCM (4 mL) was added BBr₃ (603 mg, 2.41 mmol, 231 μL, 5.0 eq) at 0° C. The mixture was stirred at 20° C. for 1 h. The reaction mixture was quenched by addition 5 mL H₂O at 0° C., the reaction mixture was concentrated under reduced pressure to remove DCM. The residue was filtered to give 1-(5-chloro-2-hydroxyphenyl)-3-((2-(2,6-dioxopiperidin-3-yl)-1-oxoisoindolin-5-yl)methyl)urea (120 mg, 270 μmol, 56% yield) as a white solid.

Step 3. To a solution of 1-(5-chloro-2-hydroxyphenyl)-3-((2-(2,6-dioxopiperidin-3-yl)-1-oxoisoindolin-5-yl)methyl)urea (100 mg, 225 μmol, 1.0 eq), tert-butyl 2-(bromomethyl)acrylate (49.9 mg, 225 μmol, 1.0 eq) in DMF (1.5 mL) was added K₂CO₃ (93.6 mg, 677 μmol, 3.0 eq). The mixture was stirred at 20° C. for 12 h. The reaction mixture was filtered and concentrated under reduced pressure to give a residue. The residue was purified by prep-HPLC (column: Phenomenex C18 75×30 mm×3 um; mobile phase: [water (FA)-ACN]; B %: 40%-70%, 7 min) give tert-butyl 2-((4-chloro-2-(3-((2-(2,6-dioxopiperidin-3-yl)-1-oxoisoindolin-5-yl)methyl)ureido)phenoxy)methyl)acrylate (50 mg, 81 μmol, 36% yield) as a white solid.

Step 4. To a solution of tert-butyl 2-((4-chloro-2-(3-((2-(2,6-dioxopiperidin-3-yl)-1-oxoisoindolin-5-yl)methyl)ureido)phenoxy)methyl)acrylate (50 mg, 120 μmol, 1.0 eq) in DCM (1 mL) was added TFA 0.5 mL). The mixture was stirred at 20° C. for 0.5 h. The reaction mixture was filtered and concentrated under reduced pressure to give 2-((4-chloro-2-(3-((2-(2,6-dioxopiperidin-3-yl)-1-oxoisoindolin-5-yl)methyl)ureido)phenoxy)methyl)acrylic acid AQ (56 mg, TFA salt) as a white solid.

### Compound AR: (R)-1-(3-(4-amino-3-(4-phenoxyphenyl)-1H-pyrazolo[3,4-d]pyrimidin-1-yl)piperidin-1-yl)-2-(bromomethyl)prop-2-en-1-one

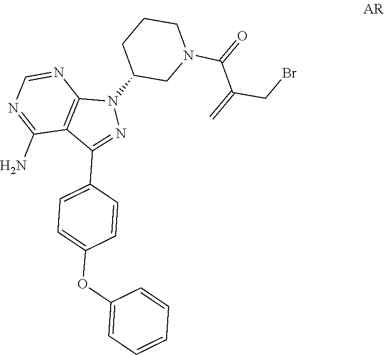 [SMILES: C=C(CBr)C(=O)N1CCC[C@@H](n2nc(-c3ccc(Oc4ccccc4)cc3)c3c(N)ncnc32)C1]

Compound AR is a known compound per J. Am. Chem. Soc., 2021, 143, 4979.

### Compound AS: 1-(3-chloro-2-hydroxyphenyl)-3-((2-(2,6-dioxopiperidin-3-yl)-1-oxoisoindolin-5-yl)methyl)urea

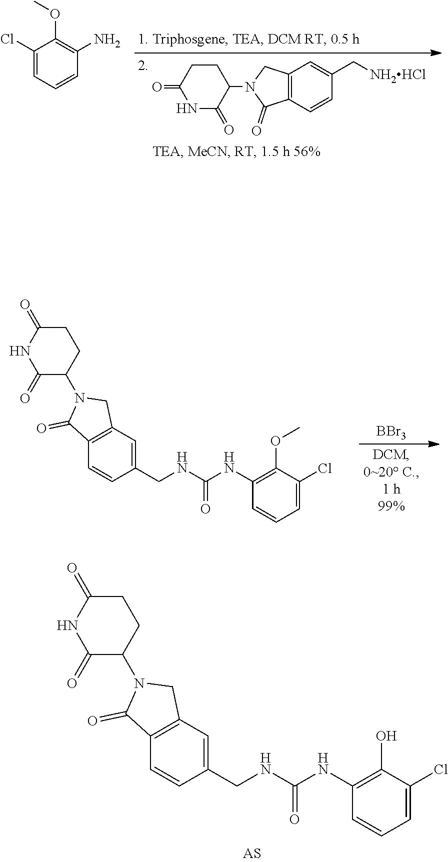

Step 1. To a solution of triphosgene (890 mg, 3.00 mmol) in DCM (3.0 mL) cooled to 0° C. was added dropwise a solution of 3-chloro-o-anisidine (473 mg, 3.00 mmol) in DCM (3.0 mL) followed by a dropwise addition of triethylamine (0.90 mL, 6.42 mmol) in DCM (3.0 mL). The reaction mixture was stirred at rt for 30 min. The volatiles were evaporated under reduced pressure. The obtained residue was added to a mixture of 3-(5-aminomethyl-1-oxo-1,3-dihydro-isoindol-2-yl)-piperidine-2,6-dione; hydrochloride (700 mg, 2.26 mmol) and triethylamine (0.63 mL, 4.52 mmol) in MeCN (15 mL) at rt. The reaction mixture was stirred at rt for 1.5 h. An aqueous solution of HCl (1 M, 15 mL) was added, and the mixture was stirred at rt for 10 min. The precipitate was collected by filtration, washed with HCl (1 M, 10 mL) and dried under vacuum to afford the title compound 1-(3-chloro-2-methoxyphenyl)-3-((2-(2,6-dioxopiperidin-3-yl)-1-oxoisoindolin-5-yl)methyl)urea (575 mg, 56%) as a solid. LC-MS Method A: MS (ES⁺): Rt=1.37 min, m/z=457.1 [M+H]⁺. ¹H NMR (DMSO-d6, 400 MHz): δH 11.00 (1H, s), 8.36 (1H, s), 8.16 (1H, d, J=8.0 Hz), 7.71 (1H, d, J=7.8 Hz), 7.52-7.53 (2H, m), 7.46 (1H, d, J=7.9 Hz), 6.97-7.04 (2H, m), 5.11 (1H, dd, J=13.3, 5.0 Hz), 4.43-4.47 (3H, m), 4.32 (1H, d, J=17.3 Hz), 3.76 (3H, s), 2.90 (1H, d, J=14.9 Hz), 2.59 (1H, d, J=17.4 Hz), 2.38 (1H, d, J=13.8 Hz), 2.00 (1H, s).

Step 2. To a solution of 1-(3-chloro-2-methoxyphenyl)-3-((2-(2,6-dioxopiperidin-3-yl)-1-oxoisoindolin-5-yl)methyl)urea (575 mg, 1.26 mmol) in DCM (11 mL) was added boron a solution of BBr₃ in DCM (1 M, 5.03 mL, 5.03 mmol). The reaction mixture was stirred at 0° C. for 1 h and then quenched by adding water (20 mL) slowly at 0° C. DCM was evaporated under reduced pressure. The precipitates were collected by filtration, washed with water, and dried under vacuum to afford the title compound 1-(3-chloro-2-hydroxyphenyl)-3-((2-(2,6-dioxopiperidin-3-yl)-1-oxoisoindolin-5-yl)methyl)urea AS (550 mg, 99%) as a solid. LC-MS Method C: MS (ES⁺): Rt=2.55 min, m/z=443.1 [M+H]⁺. ¹H NMR (DMSO-d6, 400 MHz): δH 10.99 (1H, s), 9.94 (1H, s), 8.39 (1H, s), 7.77 (1H, d, J=8.2 Hz), 7.70 (1H, d, J=7.8 Hz), 7.52 (2H, s), 7.44 (1H, d, J=7.9 Hz), 6.95 (1H, d, J=8.0 Hz), 6.77 (1H, t, J=8.1 Hz), 5.11 (1H, dd, J=13.3, 5.0 Hz), 4.43-4.47 (3H, m), 4.31 (1H, d, J=17.3 Hz), 2.87-2.91 (1H, m), 2.59 (1H, m), 2.36-2.39 (1H, m), 2.00 (1H, m).

### Compound AT: 1-(3-chloro-4-methylphenyl)-3-((2-(1-(3,5-difluoro-4-hydroxybenzyl)-2,6-dioxopiperidin-3-yl)-1-oxoisoindolin-5-yl)methyl)urea

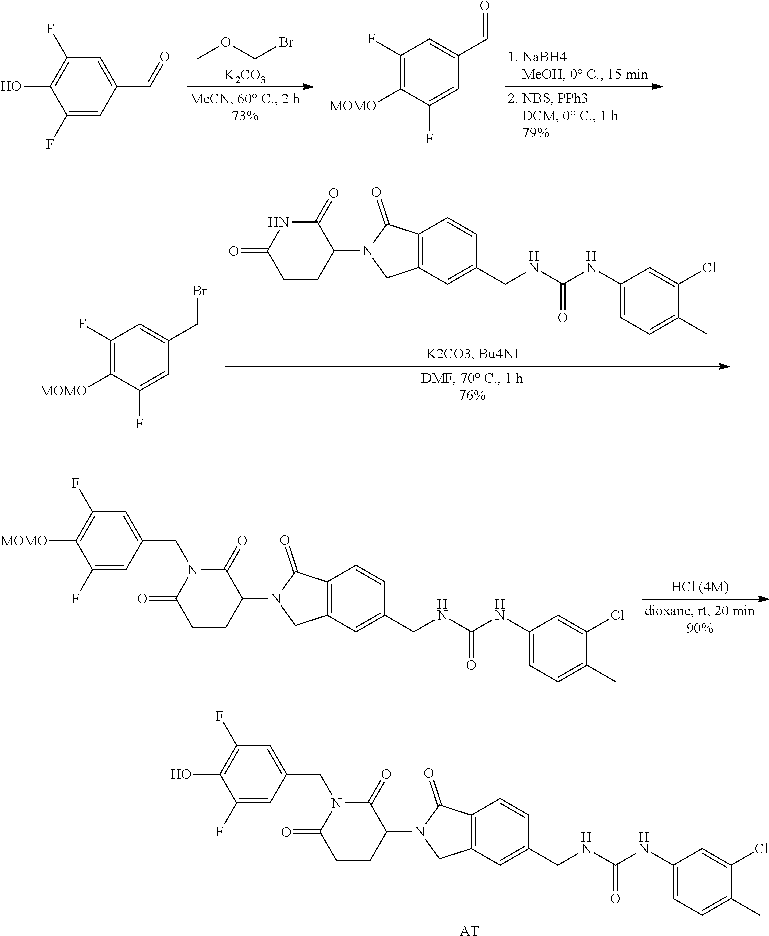

Step 1. To a solution of 3,5-difluoro-4-hydroxybenzaldehyde (1.50 g, 9.49 mmol) and bromomethyl methyl ether (0.852 mL, 10.4 mmol) in MeCN (23 mL) was added K₂CO₃ (3.93 g, 28.5 mmol) at rt. The reaction mixture was heated to 60° C. for 2 h and then cooled to rt. The volatiles were evaporated under reduced pressure. Water (20 mL) and EtOAc (20 mL) were added, and the layers were separated. The aqueous phase was extracted with EtOAc (2×50 mL). The organic layers were combined, dried over Na₂SO₄, filtered, and concentrated under reduced pressure. The crude product was purified by column chromatography on silica gel using a gradient of 0-20% EtOAc in hexane to afford 2,6-difluoro-4-(methoxymethoxy)benzaldehyde (1.40 g, 73%) as an oil. LC-MS Method A: MS (ES⁺): Rₜ=1.38 min, m/z=203.1 [M+H]⁺.

Step 2. Step 1: to a solution of 2,6-difluoro-4-(methoxymethoxy)benzaldehyde (1.40 g, 6.93 mmol) in MeOH (76 mL) cooled to 0° C. was added NaBH₄ (197 mg, 5.19 mmol). The reaction mixture was stirred at 0° C. for 15 min. Water (10 mL) was added at 0° C. The volatiles were evaporated under reduced pressure. Water (50 mL) and EtOAc (100 mL) were added, and the mixture was stirred at rt for 1 h and then the layers were separated. The aqueous layer was extracted with EtOAc (3×100 mL). The organic layers were combined, dried over Na₂SO₄, filtered, and concentrated under reduced pressure to afford the benzylic alcohol intermediate as a solid. The crude product was used in the next step without further purification. To a solution of the crude benzylic alcohol (1.40 g, 6.86 mmol) obtained in DCM (75 mL) cooled to 0° C. were sequentially added PPh₃ (2.16 g, 8.23 mmol) and NBS (1.48 g, 8.23 mmol). The reaction mixture was stirred at 0° C. for 1 h. The reaction mixture was neutralized with a saturated solution of NaHCO₃ and the layers were separated. The aqueous layer was extracted with DCM (3×30 mL). The organic layers were combined, dried over Na₂SO₄, filtered, and concentrated under reduced pressure. The crude product was purified by column chromatography on silica gel using a gradient of 0-10% EtOAc in hexane to afford 2-(bromomethyl)-1,3-difluoro-5-(methoxymethoxy)benzene (1.45 g, 79%) as a solid. LC-MS Method A: MS (ES⁺): Rt=1.85 min, m/z=no ionization. ¹H NMR (CHCl₃-d, 400 MHz): δH 6.93-6.98 (2H, m), 5.16 (2H, s), 4.37 (2H, s), 3.59 (3H, s).

Step 3. A solution of 2-(bromomethyl)-1,3-difluoro-5-(methoxymethoxy)benzene (757 mg, 2.84 mmol) in DMF (21 mL) was added to a mixture of 1-(3-chloro-4-methylphenyl)-3-((2-(2,6-dioxopiperidin-3-yl)-1-oxoisoindolin-5-yl)methyl)urea (500 mg, 1.13 mmol) and K₂CO₃ (313 mg, 2.27 mmol). Bu₄NI (127 mg, 0.34 mmol) was then added. The reaction mixture was heated to 70° C. for 1 h and then cooled to rt. The volatiles were evaporated under reduced pressure. Water (20 mL) and EtOAc (20 mL) were added, and the layers were separated. The aqueous layer was extracted with EtOAc (3×20 mL). The organic layers were combined, dried over Na₂SO₄, filtered, and concentrated under reduced pressure. The crude product was purified by column chromatography on silica gel using a gradient of 0-1% ⁱPrOH in EtOAc to afford 1-(3-chloro-4-methylphenyl)-3-((2-(1-(3,5-difluoro-4-(methoxymethoxy)benzyl)-2,6-dioxopiperidin-3-yl)-1-oxoisoindolin-5-yl)methyl)urea (0.540 g, 76%) as a solid. LC-MS Method A: MS (ES⁺): Rt=2.00 min, m/z=627.2 [M+H]⁺.

Step 4. A mixture of 1-(3-chloro-4-methylphenyl)-3-((2-(1-(3,5-difluoro-4-(methoxymethoxy)benzyl)-2,6-dioxopiperidin-3-yl)-1-oxoisoindolin-5-yl)methyl)urea 5 (540 mg, 0.861 mmol) and 4 M HCl solution in dioxane (20 mL, 80 mmol) was stirred at rt for 30 min. The volatiles were evaporated under reduced pressure. The crude product was purified by reverse phase chromatography (C18) using a gradient of 0-100 MeCN in water (containing 0.1% formic acid) to afford 1-(3-chloro-4-methylphenyl)-3-((2-(1-(3,5-difluoro-4-hydroxybenzyl)-2,6-dioxopiperidin-3-yl)-1-oxoisoindolin-5-yl)methyl)urea AT (450 mg, 90%) as a solid. LC-MS Method C: MS (ES⁺): Rt=3.29 min, m/z=583.1 [M+H]⁺. ¹H NMR (DMSO-d₆, 400 MHz): δ_{H} 10.08 (1H, s), 8.77 (1H, s), 7.69 (1H, d, J=7.8 Hz), 7.66 (1H, s), 7.52 (1H, s), 7.44 (1H, d, J=7.9 Hz), 7.18 (1H, d, J=8.3 Hz), 7.12 (1H, d, J=8.5 Hz), 6.91 (2H, d, J=8.0 Hz), 6.80 (1H, t, J=5.9 Hz), 5.29 (1H, dd, J=13.3, 5.0 Hz), 4.66-4.76 (2H, m), 4.28-4.49 (4H, m), 3.20-3.10 (1H, m), 2.82-2.75 (1H, m), 2.49-2.80 (1H, m), 2.22 (3H, s), 2.05 (1H, s).

### Compound AU: 2-(bromomethyl)-N-(2-((2-(dimethylamino)ethyl)(methyl)amino)-4-methoxy-5-((4-(1-methyl-1H-indol-3-yl)pyrimidin-2-yl)amino)phenyl)acrylamide

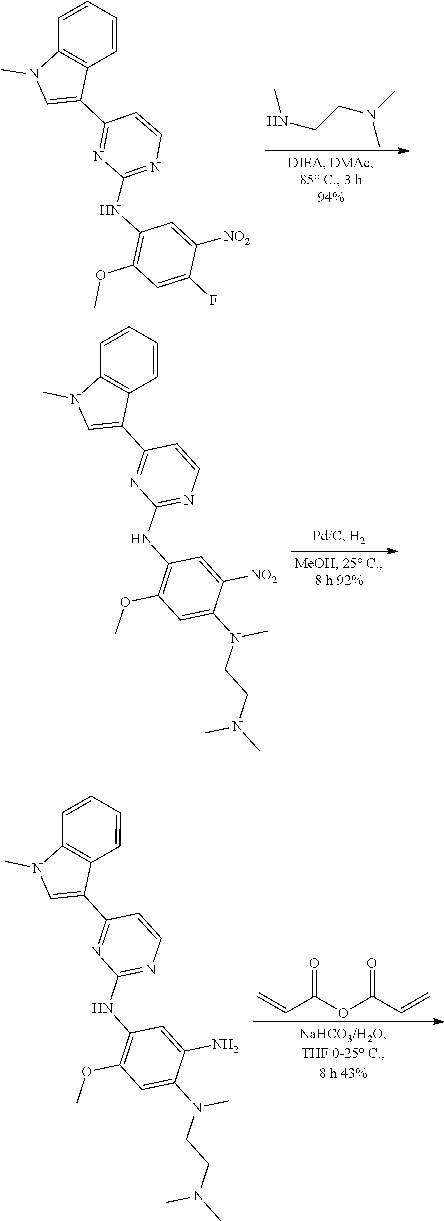

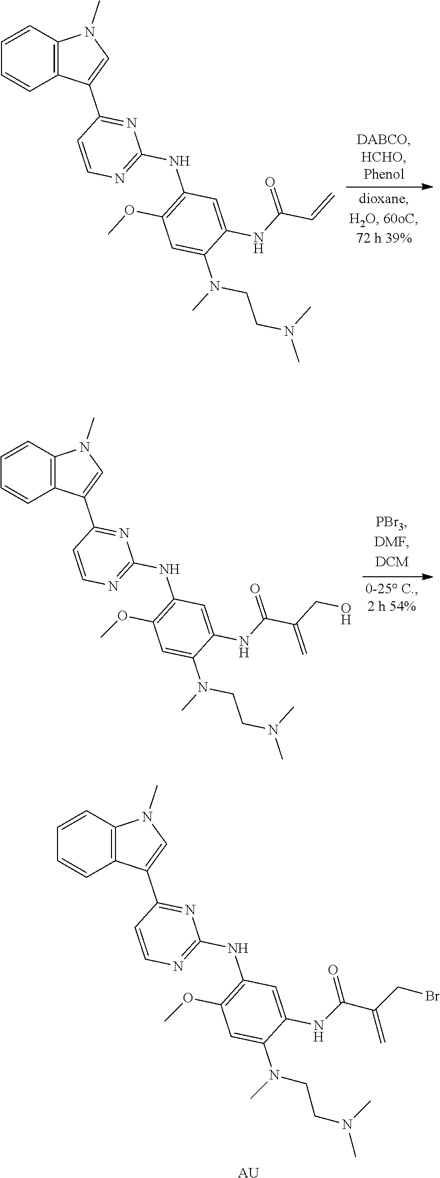 [SMILES: C=CC(=O)Nc1cc(Nc2nccc(-c3cn(C)c4ccccc34)n2)c(OC)cc1N(C)CCN(C)C ; C=C(CO)C(=O)Nc1cc(Nc2nccc(-c3cn(C)c4ccccc34)n2)c(OC)cc1N(C)CCN(C)C ; C=C(CBr)C(=O)Nc1cc(Nc2nccc(-c3cn(C)c4ccccc34)n2)c(OC)cc1N(C)CCN(C)C ; "AU"]

Step 1. To a solution of N-(4-fluoro-2-methoxy-5-nitro-phenyl)-4-(1-methylindol-3-yl)pyrimidin-2-amine (5.00 g, 12.7 mmol, 1 eq) and N,N′,N′-trimethylethane-1,2-diamine (1.43 g, 14.0 mmol, 1.82 mL, 1.1 eq) in DMAC (100 mL) was added DIEA (4.11 g, 31.8 mmol, 5.53 mL, 2.5 eq) at 25° C. The resulting mixture was stirred at 85° C. for 8 hours. The reaction solution was diluted with 900 mL H₂O, then filtered to get the filter cake, which was concentrated under reduced pressure to get crude product N4-[2-(dimethylamino)ethyl]-2-methoxy-N4-methyl-N1-[4-(1-methylindol-3-yl)pyrimidin-2-yl]-5-nitro-benzene-1,4-diamine (5.70 g, 12.0 mmol, 94% yield) as a yellow solid.

Step 2. To a solution of N4-[2-(dimethylamino)ethyl]-2-methoxy-N4-methyl-N1-[4-(1-methylindol-3-yl)pyrimidin-2-yl]-5-nitro-benzene-1,4-diamine (5.50 g, 11.6 mmol, 1 eq) in MeOH (200 mL) was added 10% wet Pd/C (2 g, 10% purity, w/w). The suspension was degassed under vacuum and purged with H₂ several times. The mixture was stirred under H₂ (15 psi) at 25° C. for 8 hours. The reaction mixture was filtered and the filter was concentrated to give crude product Ni-[2-(dimethylamino)ethyl]-5-methoxy-N1-methyl-N4-[4-(1-methylindol-3-yl)pyrimidin-2-yl]benzene-1,2,4-triamine (4.9 g, 11.0 mmol, 95% yield) as a black-brown solid.

Step 3. To a mixture of N1-[2-(dimethylamino)ethyl]-5-methoxy-N1-methyl-N4-[4-(1-methylindol-3-yl)pyrimidin-2-yl]benzene-1,2,4-triamine (1.00 g, 2.24 mmol, 1 eq) in THF (5 mL) was added NaHCO₃/H₂O (5 mL) and then added a solution of prop-2-enoyl prop-2-enoate (368 mg, 2.92 mmol, 1.3 eq) in THF (1.25 mL) at 0° C. The resulting mixture was then stirred at 25° C. for 8 hours. The reaction mixture was concentrated under reduced pressure to remove solvent. The residue was diluted with Ethyl acetate 300 mL and washed with H₂O 300 mL (100 mL×3), dried over anhydrous Na₂SO₄, filtered and concentrated under reduced pressure to give a residue. The residue was purified by column chromatography (SiO₂, Ethyl acetate/Methanol=5/1 to 1/2) to give product N-[2-[2-(dimethylamino)ethyl-methyl-amino]-4-methoxy-5-[[4-(1-methylindol-3-yl)pyrimidin-2-yl]amino]phenyl]prop-2-enamide (480 mg, 961 μmol, 43% yield) as a brown solid.

Step 4. To a solution of N-[2-[2-(dimethylamino)ethyl-methyl-amino]-4-methoxy-5-[[4-(1-methylindol-3-yl)pyrimidin-2-yl]amino]phenyl]prop-2-enamide (480 mg, 961 μmol, 1 eq), DABCO (140 mg, 1.25 mmol, 137 μL, 1.3 eq) and HCHO (1.56 g, 19.2 mmol, 1.43 mL, 37% purity, 20 eq) in dioxane (7.5 mL) and H₂O (2.5 mL) was added Phenol (40.7 mg, 432 μmol, 38.0 μL, 0.45 eq), the mixture solution was heated to 60° C. and stirred for 72 hours. The reaction mixture was concentrated to give a residue. The residue was purified by prep-HPLC (column: Phenomenex C18 75×30 mm×3 um; mobile phase: [water(FA)-ACN]; B %: 8%-38%, 7 min) to give product N-[2-[2-(dimethylamino)ethyl-methyl-amino]-4-methoxy-5-[[4-(1-methylindol-3-yl)pyrimidin-2-yl]amino]phenyl]-2-(hydroxymethyl)prop-2-enamide (200 mg, 378 μmol, 39% yield) was obtained as a yellow solid.

Step 5. To a solution of N-[2-[2-(dimethylamino)ethyl-methyl-amino]-4-methoxy-5-[[4-(1-methylindol-3-yl)pyrimidin-2-yl]amino]phenyl]-2-(hydroxymethyl)prop-2-enamide (200 mg, 378 μmol, 1 eq) in DCM (2 mL) was added DMF (27.6 mg, 378 μmol, 29.1 μL, 1.0 eq) and PBr₃ (112 mg, 415 μmol, 1.1 eq) at 0° C. The mixture was stirred at 25° C. for 2 hours. The reaction mixture was quenched by addition of 10 mL H₂O at 25° C. The reaction mixture was concentrated by lyophilization to give crude product 2-(bromomethyl)-N-[2-[2-(dimethylamino)ethyl-methyl-amino]-4-methoxy-5-[[4-(1-methylindol-3-yl)pyrimidin-2-yl]amino]phenyl]prop-2-enamide AU (120 mg, 203 μmol, 54% yield) as a yellow solid.

### Compound AV: 2-((S)-1-(2-(bromomethyl)acryloyl)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

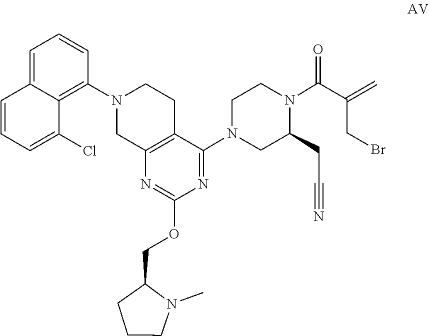 [SMILES: C=C(CBr)C(=O)N1CCN(c2nc(OC[C@@H]3CCCN3C)nc3c2CCN(c2cccc4cccc(Cl)c24)C3)C[C@@H]1CC#N]

To a solution of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-[2-(hydroxymethyl)prop-2-enoyl]piperazin-2-yl]acetonitrile (20 mg, 32.46 μmol, 1 eq) in DCM (0.5 mL) was added PBr₃ (8.79 mg, 32.46 μmol, 60 μL, 1 eq) at 0° C. The reaction mixture was stirred at 0° C. for 0.5 h. After concentrated, Compound 2-[(2S)-1-[2-(bromomethyl)prop-2-enoyl]-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile AV (22 mg, 99% yield) as a colorless liquid.

### Compound AW: 1-(4,5-difluoro-2-hydroxyphenyl)-3-((2-(2,6-dioxopiperidin-3-yl)-1-oxoisoindolin-5-yl)methyl)urea

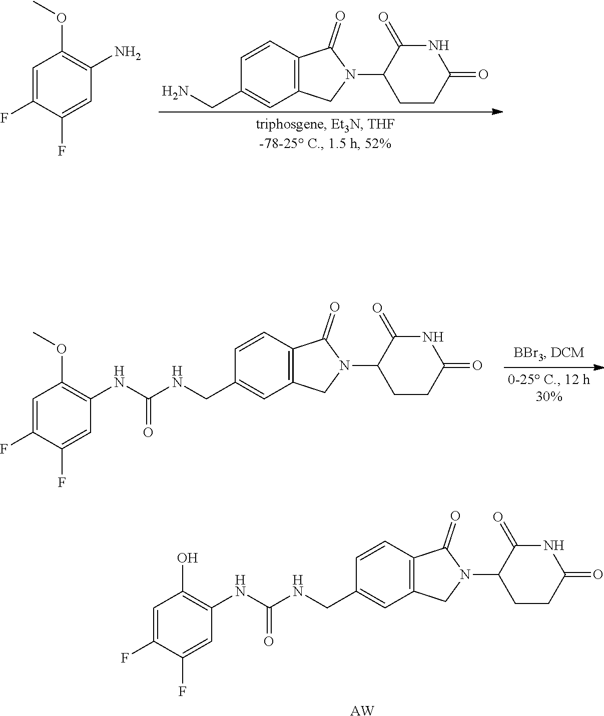 [SMILES: COc1cc(F)c(F)cc1N ; NCc1ccc2c(c1)CN(C1CCC(=O)NC1=O)C2=O ; COc1cc(F)c(F)cc1NC(=O)NCc1ccc2c(c1)CN(C1CCC(=O)NC1=O)C2=O ; O=C1CCC(N2Cc3cc(CNC(=O)Nc4cc(F)c(F)cc4O)ccc3C2=O)C(=O)N1 ; "AW"]

Step 1. To a solution of triphosgene (104 mg, 350 μmol, 0.75 eq) in THF (6 mL) was added Et₃N (636 mg, 6.28 mmol, 875 μL, 10 eq) and 4,5-difluoro-2-methoxy-aniline (0.1 g, 628.40 μmol, 1 eq). The mixture was stirred at −78° C. for 0.5 h. Then 3-[5-(aminomethyl)-1-oxo-isoindolin-2-yl] piperidine-2,6-dione (195 mg, 628.40 μmol, 1 eq, HCl salt) in THF (1 mL) was added. The mixture was stirred at −78° C. for 0.5 h. The mixture was stirred at 25° C. for 0.5 h. The mixture was added 50 mL H₂O and extracted with EA (50 mL×3). The combined organic layers were filtered and concentrated under reduced pressure to give a residue. The residue was purified by prep-HPLC (column: Phenomenex C18 75×30 mm×3 um; mobile phase: [water(FA)-ACN]; B %: 25%-55%, 7 min) to give compound 1-(4,5-difluoro-2-methoxy-phenyl)-3-[[2-(2,6-dioxo-3-piperidyl)-1-oxo-isoindolin-5-yl]methyl]urea (150 mg, 52% yield) as a white solid.

Step 2. To a solution of 1-(4,5-difluoro-2-methoxy-phenyl)-3-[[2-(2,6-dioxo-3-piperidyl)-1-oxo-isoindolin-5-yl] methyl] urea (100 mg, 218.14 μmol, 1 eq) in DCM (3 mL) was added BBr₃ (273.25 mg, 1.09 mmol, 105 μL, 5 eq) at 0° C. The mixture was stirred at 25° C. for 12 h. The reaction mixture was added 0.5 mL H₂O and concentrated under reduced pressure to remove solvent. The residue was purified by prep-HPLC (column: Phenomenex C18 75×30 mm×3 um; mobile phase: [water (FA)-ACN]; B %: 15%-45%, 7 min) to give compound 1-(4,5-difluoro-2-hydroxy-phenyl)-3-[[2-(2,6-dioxo-3-piperidyl)-1-oxo-isoindolin-5-yl]methyl]urea AW (30 mg, 30% yield) as a white solid. ¹H NMR (400 MHz, DMSO-d₆): δ 7.80 (s, 1H), 4.19-4.28 (m, 4H), 2.34 (s, 3H), 1.25-1.31 (m, 6H). LC-MS: MS (ES⁺): RT=1.97 min, m/z=445.1 [M+H⁺]; LC-MS Method AB10.

### Compound AX: 1-((2-(2,6-dioxopiperidin-3-yl)-1-oxoisoindolin-5-yl)methyl)-3-(2-hydroxybenzyl)urea

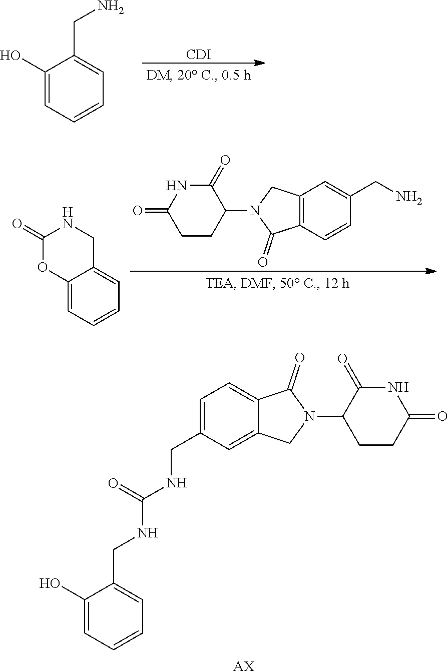

Step 1. To a solution of 2-(aminomethyl)phenol (50 mg, 406 μmol, 1.0 eq) and TEA (123 mg, 1.2 mmol, 169 μL, 3.0 eq) in DMF (1 mL) was added CDI (79 mg, 487 μmol, 1.2 eq). Then the reaction mixture was stirred at 20° C. for 0.5 h. The crude product 3,4-dihydro-1,3-benzoxazin-2-one (60 mg, 406 μmol) was used into the next step directly.

Step 2. To a solution of 3,4-dihydro-1,3-benzoxazin-2-one (60 mg, 402 μmol, 1.0 eq) and 3-[5-(aminomethyl)-1-oxo-isoindolin-2-yl]piperidine-2,6-dione (125 mg, 402 μmol, 1.0 eq, HCl) in DMF (2 mL) was added TEA (81 mg, 805 μmol, 112 μL, 2.0 eq). The mixture was stirred at 50° C. for 12 h. The reaction mixture was concentrated to give a residue. The residue was purified by prep-HPLC (TFA condition; column: Phenomenex luna C18 150×25 mm×10 um; mobile phase: [water(FA)-ACN]; B %: 15%-45%, 15 min). Compound 1-[[2-(2,6-dioxo-3-piperidyl)-1-oxo-isoindolin-5-yl]methyl]-3-[(2-hydroxyphenyl)methyl]urea AX (26.79 mg, 62.18 μmol, 15% yield, 98.1% purity) was obtained as a white solid. LC-MS: MS (ES⁺): RT=1.991 min, m/z=423.2 [M+H⁺]; LC-MS Method AB05. ¹H NMR: (400 MHz, CD₃Cl) δ=7.75-7.73 (d, J=7.6 Hz, 1H), 7.47 (d, J=2.0 Hz, 1H), 7.44-7.42 (d, J=8.0 Hz, 1H), 7.16-7.14 (d, J=7.6 Hz, 1H), 7.09 (m, 1H), 6.78-6.76 (m, 2H), 5.13 (m, 1H), 4.45 (m, 4H), 4.29 (s, 2H), 2.94-2.91 (m, 1H), 2.90-2.88 (m, 1H), 2.51-2.47 (m, 1H), 2.20 (m, 1H).

### Compound AY: 1-(3,4-difluoro-5-hydroxyphenyl)-3-((2-(2,6-dioxopiperidin-3-yl)-1-oxoisoindolin-5-yl)methyl)urea

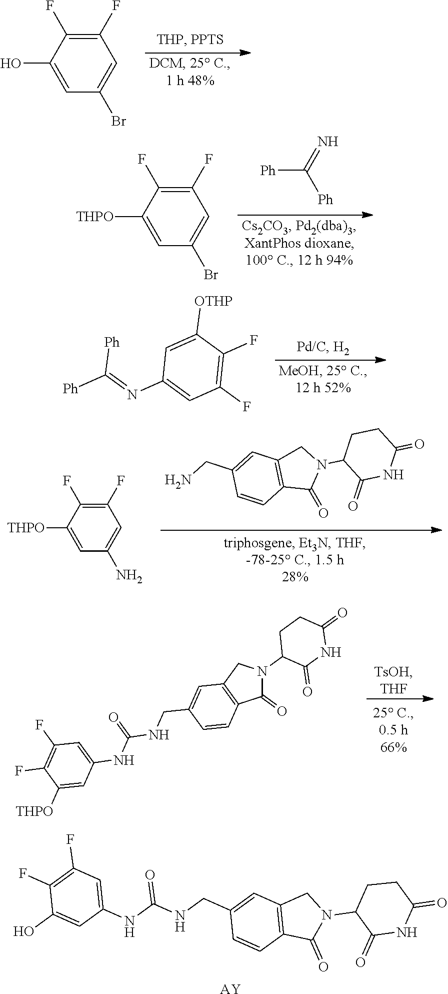

Step 1. To a solution of 5-bromo-2,3-difluoro-phenol (10 g, 47.85 mmol, 1.0 eq) and 3,4-dihydro-2H-pyran (8.05 g, 95.70 mmol, 8.75 mL, 2.0 eq) in DCM (20 mL) was added PPTS (601 mg, 2.39 mmol, 0.05 eq). The mixture was stirred at 25° C. for 1 h. The residue was purified by silica chromatography (PE:EA=20:1-10:1) to afford 6-(5-bromo-2,3-difluoro-phenoxy)-3,4-dihydro-2H-pyran (6.7 g, 48% yield). ¹H NMR (400 MHz, CDCl₃): δ 7.22-7.25 (m, 1H), 6.90-7.10 (m, 1H), 5.47-5.48 (m, 1H), 3.68-3.72 (m, 1H), 3.51-3.58 (m, 1H), 1.76-1.90 (m, 4H), 1.60-1.75 (m, 1H).

Step 2. To a solution of 2-(5-bromo-2,3-difluoro-phenoxy)tetrahydropyran (3.00 g, 10.24 mmol, 1.0 eq), diphenylmethanimine (2.78 g, 15.4 mmol, 1.5 eq) in dioxane (30 mL) was added Cs₂CO₃ (10.0 g, 30.7 mmol, 3.0 eq) and Pd₂(dba)₃ (469 mg, 512 μmol, 0.05 eq), (5-diphenylphosphanyl-9,9-dimethyl-xanthen-4-yl)-diphenyl-phosphane (592 mg, 1.02 mmol, 0.1 eq). The mixture was stirred at 100° C. for 12 h. The reaction mixture was concentrated under reduced pressure to remove solvent. The residue was purified by prep-TLC (SiO₂, Petroleum ether: Ethyl acetate=10:1) to give compound N-(3,4-difluoro-5-tetrahydropyran-2-yloxy-phenyl)-1,1-diphenyl-methanimine (3.8 g, 94% yield) as a white solid. ¹H NMR (400 MHz, CDCl₃): δ 7.81-7.83 (m, 1H), 7.71-7.75 (m, 2H), 7.57-7.65 (m, 1H), 7.47-7.53 (m, 3H), 7.41 (s, 1H), 7.09-7.15 (m, 2H), 6.36-6.38 (m, 1H), 6.23-6.28 (m, 1H), 5.16-5.18(m, 1H), 3.72-3.78 (m, 1H), 3.42-3.54 (m, 1H), 1.90-2.04 (m, 1H), 1.75-1.88 (m, 2H), 1.57-1.69 (m, 3H).

Step 3. To a solution of N-(3,4-difluoro-5-tetrahydropyran-2-yloxy-phenyl)-1,1-diphenyl-methanimine (2.5 g, 6.35 mmol, 1.0 eq) in MeOH (10 mL) was added Pd/C (1.5 g, 10% purity) under H₂ (15 Psi). The mixture was stirred at 25° C. for 12 h under H₂ (15 psi). The reaction mixture was filtered and concentrated under reduced pressure to give a residue. The residue was purified by column chromatography (SiO₂, Petroleum ether/Ethyl acetate=60/1 to 30/1) to give compound 3,4-difluoro-5-tetrahydropyran-2-yloxy-aniline (0.76 g, 52% yield) as a white solid. ¹H NMR (400 MHz, CDCl₃): δ 6.28-6.39 (m, 1H), 6.11-6.31 (m, 1H), 5.33-5.45 (m, 1H), 3.90-3.96 (m, 1H), 3.60-3.65 (m, 1H), 1.86-2.06 (m, 3H), 1.61-1.75 (m, 3H).

Step 4. To a solution of triphosgene (510 mg, 1.72 mmol, 2.63 eq) in THF (30 mL) was added 3-[5-(aminomethyl)-1-oxo-isoindolin-2-yl]piperidine-2,6-dione (203 mg, 654 μmol, 1.0 eq, HCl salt) and ET₃N (662 mg, 6.54 mmol, 10.0 eq). The mixture was stirred at −78° C. for 0.5 h. Then 3,4-difluoro-5-tetrahydropyran-2-yloxy-aniline (0.15 g, 654 μmol, 1.0 eq) was added. The mixture was stirred at −78° C. for 0.5 h. The mixture was stirred at 25° C. for 0.5 h. The residue was added 50 mL H₂O and extracted with EA (50 mL×3). The combined organic layers were filtered and concentrated under reduced pressure to give a residue. The residue was purified by prep-HPLC (column: Waters Xbridge 150×25 mm×5 um; mobile phase: [water(NH₄HCO₃)-ACN]; B %: 30%-60%, 9 min) to give compound 1-(3,4-difluoro-5-tetrahydropyran-2-yloxy-phenyl)-3-[[2-(2,6-dioxo-3-piperidyl)-1-oxo-isoindolin-5-yl]methyl]urea (0.1 g, 28% yield) as a white solid.

Step 5. To a solution of 1-(3,4-difluoro-5-tetrahydropyran-2-yloxy-phenyl)-3-[[2-(2,6-dioxo-3-piperidyl)-1-oxo-isoindolin-5-yl] methyl] urea (70 mg, 0.13 mmol, 1.0 eq) in THF (1 mL) was added TsOH (68 mg, 0.40 mmol, 3.0 eq). The mixture was stirred at 25° C. for 0.5 h. The residue was purified by prep-HPLC (column: Phenomenex Synergi Polar-RP 100×25 mm×4 um; mobile phase: [water(TFA)-ACN]; B %: 30%-50%, 7 min) to give compound 1-(3,4-difluoro-5-hydroxy-phenyl)-3-[[2-(2,6-dioxo-3-piperidyl)-1-oxo-isoindolin-5-yl]methyl]urea AY (40 mg, 66% yield) as a white solid. ¹H NMR (400 MHz, DMSO-d₆): δ 10.87-11.10 (m, 1H), 10.07-10.41 (m, 1H), 8.72 (s, 1H), 7.69 (d, J=7.82 Hz, 1H), 7.35-7.58 (m, 2H), 6.58-7.04 (m, 3H), 5.10 (m, 1H), 4.32-4.52 (m, 4H), 3.67-3.78 (m, 2H), 2.79-3.02 (m, 1H), 1.90-2.09 (m, 1H). LC-MS: MS (ES⁺): RT=2.14 min, m/z=445.1 [M+H⁺]; Method ABO1.

### Compound AZ: 1-(2-(2-aminoethyl)-5-chlorophenyl)-3-((2-(2,6-dioxopiperidin-3-yl)-1-oxoisoindolin-5-yl)methyl)urea

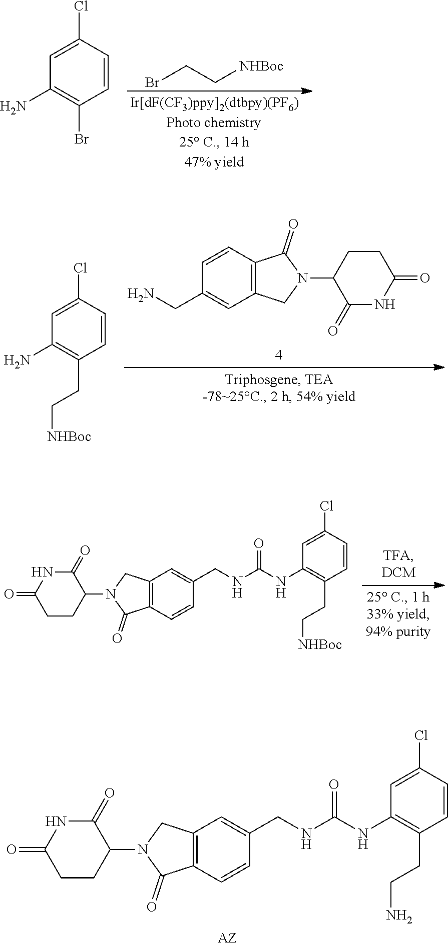

Step 1. A mixture of 2-bromo-5-chloro-aniline (1.5 g, 7.3 mmol, 1.0 equiv), tert-butyl N-(2-bromoethyl)carbamate (2.4 g, 10.9 mmol, 1.5 equiv), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (82 mg, 73 umol, 0.01 equiv), 4-tert-butyl-2-(4-tert-butyl-2-pyridyl) pyridine; dichloronickel (145 mg, 363 umol, 0.05 equiv), TTMSS (1.8 g, 7.3 mmol, 2.2 mL, 1.0 equiv), Na₂CO₃ (1.5 g, 14.5 mmol, 2.0 equiv) in DME (15 mL) was degassed and purged with N₂ for 3 times. The reaction was stirred and irradiated with a 34 W blue LED lamp (7 cm away), with cooling fan to keep the reaction temperature at 25° C. for 14 h. The mixture was concentrated to give the residue. The residue was purified by prep-HPLC (column: Phenomenex luna C18 (250*70 mm, 10 um); mobile phase: [water (NH₄HCO₃)-ACN]; B %: 34%-70%, 25 min) to give the tert-butyl N-[2-(2-amino-4-chloro-phenyl) ethyl] carbamate (923 mg, 3.4 mmol, 47% yield) as a yellow solid.

Step 2. To a solution of Triphosgene (80 mg, 180 umol, 0.5 equiv) in THF (5 mL) stirred at −78° C. under N₂ protection, then tert-butyl N-[2-(2-amino-4-chloro-phenyl)ethyl]carbamate (100 mg, 369 umol, 1.0 equiv) and TEA (374 mg, 3.7 mmol, 514 μL, 10.0 equiv) was added to the mixture and stirred at −78° C. for 30 min. Then the 3-[5-(aminomethyl)-1-oxo-isoindolin-2-yl]piperidine-2,6-dione (100 mg, 369 umol, 1.0 equiv) was added to the mixture at −78° C. and stirred for 30 min. Then the mixture was allowed stirred at 25° C. for 1 h under N₂ protection. The mixture was poured into H₂O (10 mL) and extracted with DCM (10 mL×3)), then the mixture was washed with brine and concentrated to give the residue. The residue was purified by prep-HPLC (column: Phenomenex C18 150*25 mm*10 um; mobile phase: [water (NH₄HCO₃)-ACN]; B %: 29%-59%, 8 min) and again by prep-HPLC (column: Phenomenex luna C18 150*25 mm*10 um; mobile phase: [water (FA)-ACN]; B %: 35%-65%, 15 min) to give the tert-butyl N-[2-[4-chloro-2-[[2-(2,6-dioxo-3-piperidyl)-1-oxo-isoindolin-5-yl]methylcarbamoylamino]phenyl]ethyl]carbamate (113 mg, 198umol, 54% yield) as a light yellow solid. ¹H NMR: (400 MHz, DMSO-d6) δ=8.06-8.02 (m, 2H), 7.73-7.64 (m, 1H), 7.57-7.43 (m, 2H), 7.10 (d, J=8.4 Hz, 2H), 6.98-6.91 (m, 2H), 5.15-5.07 (m, 1H), 4.50-4.28 (m, 5H), 3.16-3.08 (m, 2H), 2.98-2.85 (m, 1H), 2.63-2.56 (m, 1H), 2.42-2.35 (m, 1H), 2.04-1.95 (m, 1H), 1.33 (s, 9H), 1.23 (s, 1H).

Step 3. A mixture of tert-butyl N-[2-[4-chloro-2-[[2-(2,6-dioxo-3-piperidyl)-1-oxo-isoindolin-5-yl]methylcarbamoylamino]phenyl]ethyl]carbamate (45 mg, 79 umol, 1.0 equiv) in DCM (3 mL), TFA (1 mL) was degassed and purged with N₂ for 3 times, and then the mixture was stirred at 25° C. for 1 h under N₂ atmosphere. The mixture was concentrated to give the residue. The residue was purified by prep-HPLC (column: Phenomenex luna C18 150*25 mm*10 um; mobile phase: [water (FA)-ACN]; B %: 1%-30%, 15 min) to give the 1-[2-(2-aminoethyl)-5-chloro-phenyl]-3-[[2-(2,6-dioxo-3-piperidyl)-1-oxo-isoindolin-5-yl]methyl]urea (13 mg, 26 umol, 34% yield, 94% purity) as a white solid. ¹H NMR: (400 MHz, DMSO-d6) δ=8.46 (s, 1H), 8.15 (d, J=2.0 Hz, 1H), 8.08 (s, 1H), 7.69 (d, J=8.0 Hz, 1H), 7.53 (s, 1H), 7.45 (d, J=8.0 Hz, 1H), 7.15 (d, J=8.0 Hz, 1H), 6.99-6.91 (m, 1H), 5.15-5.05 (m, 1H), 4.43 (d, J=4.8 Hz, 3H), 4.33 (s, 1H), 2.99-2.76 (m, 6H), 2.65-2.55 (m, 1H), 2.42-2.31 (m, 2H), 2.03-1.95 (m, 1H).

### Compound AAA: 1-(2-(3-aminopropyl)-5-chlorophenyl)-3-((2-(2,6-dioxopiperidin-3-yl)-1-oxoisoindolin-5-yl)methyl)urea

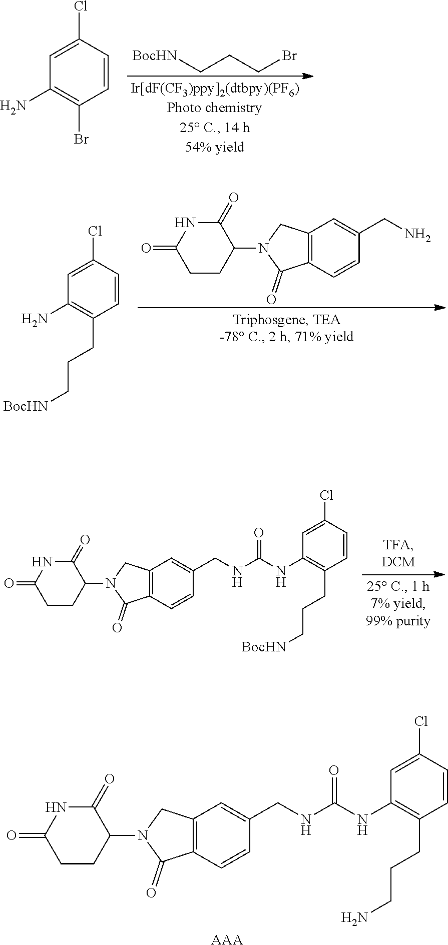

Step 1. A mixture of 2-bromo-5-chloro-aniline (1.5 g, 7.3 mmol, 1.0 equiv), tert-butyl N-(3-bromopropyl)carbamate (2.6 g, 10.9 mmol, 1.5 equiv), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (82 mg, 73 umol, 0.01 equiv), 4-tert-butyl-2-(4-tert-butyl-2-pyridyl) pyridine; dichloronickel (144 mg, 363 umol, 0.05 equiv), TTMSS (1.8 g, 7.3 mmol, 2.2 mL, 1.0 equiv), Na₂CO₃ (770 mg, 7.3 mmol, 1.0 equiv) in DME (15 mL) was degassed and purged with N₂ for 3 times. The reaction was stirred and irradiated with a 34 W blue LED lamp (7 cm away), with cooling fan to keep the reaction temperature at 25° C. for 14 h. The mixture was concentrated to give the residue. The residue was purified by prep-HPLC (column: YMC Triart C18 250*50 mm*7 um; mobile phase: [water (NH₄HCO₃)-ACN]; B %: 42%-72%, min) to give the tert-butyl N-[3-(2-amino-4-chloro-phenyl) propyl] carbamate (1.1 g, 3.9 mmol, 54% yield) as a white solid. ¹H NMR: (400 MHz, CDCl3) δ=7.28 (s, 1H), 6.96 (d, J=6.8 Hz, 1H), 6.70 (s, 1H), 4.74-4.39 (m, 1H), 3.25-3.05 (m, 2H), 2.51 (s, 1H), 1.93-1.70 (m, 2H), 1.47 (s, 9H).

Step 2. To a solution of Triphosgene (92 mg, 309 umol, 0.5 equiv) in THF (10 mL) stirred at −78° C. under N₂ protection, then tert-butyl N-[3-(2-amino-4-chloro-phenyl)propyl]carbamate (105 mg, 369 umol, 1.0 equiv) and TEA (373 mg, 3.7 mmol, 514, 10.0 equiv) was added to the mixture and stirred at −78° C. for 30 min. Then the 3-[5-(aminomethyl)-1-oxo-isoindolin-2-yl]piperidine-2,6-dione (101 mg, 369 umol, 1.0 equiv) was added to the mixture at −78° C. and stirred for 30 min. Then the mixture was allowed stirred at 25° C. for 1 h under N₂ protection. The mixture was poured into H₂O (10 mL) and extracted with DCM (10 mL×3), then the mixture was washed with brine and concentrated to give the residue. The residue was purified by prep-HPLC (column: Phenomenex C18 150*25 mm*0 um; mobile phase: [water (NH₄HCO₃)-ACN]; B %: 29%-59%, 8 min) and purified by prep-HPLC (column: Phenomenex luna C18 150*25 mm*10 um; mobile phase: [water (FA)-ACN]; B %: 32%-62%, 15 min) to give the tert-butyl N-[3-[4-chloro-2-[[2-(2,6-dioxo-3-piperidyl)-1-oxo-isoindolin-5-yl]methyl-carbamoylamino]phenyl]propyl]carbamate (154 mg, 264 umol, 71% yield) as a light yellow solid. ¹H NMR: (400 MHz, DMSO-d6) δ=8.08-7.93 (m, 2H), 7.70 (d, J=8.0 Hz, 1H), 7.53 (s, 1H), 7.48-7.41 (m, 2H), 7.13 (d, J=8.4 Hz, 1H), 6.98-6.84 (m, 2H), 5.17-5.06 (m, 1H), 4.50-4.27 (m, 4H), 3.00-2.85 (m, 3H), 2.69-2.56 (m, 1H), 2.42-2.31 (m, 2H), 2.05-1.95 (m, 1H), 1.65-1.56 (m, 2H), 1.37 (s, 9H).

Step 3. A mixture of tert-butyl N-[3-[4-chloro-2-[[2-(2,6-dioxo-3-piperidyl)-1-oxo-isoindolin-5-yl]methylcarbamoylamino]phenyl]propyl]carbamate (154 mg, 264 umol, 1.0 equiv)) in DCM (3 mL), TFA (1 mL) was degassed and purged with N₂ for 3 times, and then the mixture was stirred at 25° C. for 1 h under N₂ atmosphere. The mixture was concentrated to give the residue. The residue was purified by prep-HPLC (column: Welch Ultimate AQ-C18 150*30 mm*5 um; mobile phase: [water (HCl)-ACN]; B %: 5%-35%, 10 min) to give the 1-[2-(3-aminopropyl)-5-chloro-phenyl]-3-[[2-(2,6-dioxo-3-piperidyl)-1-oxo-isoindolin-5-yl]methyl]urea (10 mg, 20 umol, 7% yield, 99% purity) as an off-white solid. ¹H NMR: (400 MHz, DMSO-d6) δ=10.99 (s, 1H), 8.40 (s, 1H), 8.12 (s, 1H), 7.96-7.79 (m, 4H), 7.71 (d, J=8.0 Hz, 1H), 7.55 (s, 1H), 7.47 (d, J=8.0 Hz, 1H), 7.15 (d, J=8.0 Hz, 1H), 6.96 (d, J=6.8 Hz, 1H), 5.19-5.07 (m, 1H), 4.49-4.42 (m, 3H), 4.35-4.29 (m, 1H), 2.86 (d, J=10.0 Hz, 3H), 2.65 (d, J=20.0 1H), 2.55 (s, 2H), 2.38 (s, 1H), 2.04-1.98 (m, 1H), 1.82-1.75 (m, 2H).

### Compound AAB: N₂-(4-aminocyclohexyl)-9-isopropyl-N₆-(4-(pyridin-2-yl)benzyl)-9H-purine-2,6-diamine

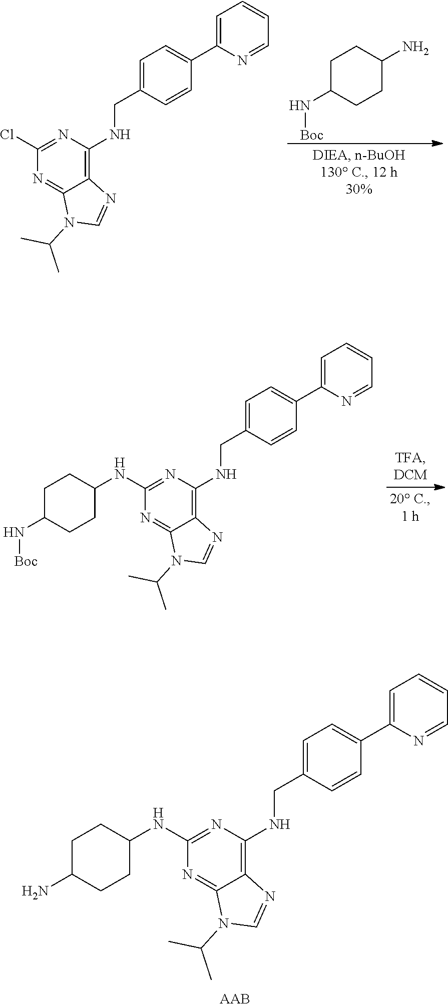 [SMILES: CC(C)n1cnc2c(NCc3ccc(-c4ccccn4)cc3)nc(Cl)nc21 ; CC(C)(C)OC(=O)NC1CCC(N)CC1 ; CC(C)n1cnc2c(NCc3ccc(-c4ccccn4)cc3)nc(NC3CCC(NC(=O)OC(C)(C)C)CC3)nc21 ; CC(C)n1cnc2c(NCc3ccc(-c4ccccn4)cc3)nc(NC3CCC(N)CC3)nc21]

Step 1. To a solution of 2-chloro-9-isopropyl-N-[[4-(2-pyridyl)phenyl]methyl]purin-6-amine (Journal of Medicinal Chemistry, 2008, vol. 51, #17, p. 5229-5242) (800 mg, 2.11 mmol, 1 equiv) and tert-butyl N-(4-aminocyclohexyl)carbamate (905 mg, 4.22 mmol, 2 equiv) in n-BuOH (5 mL) was added DIEA (1.36 g, 10.56 mmol, 1.84 mL, 5 equiv). The mixture was stirred at 130° C. for 12 h. The reaction mixture was filtered and concentrated under reduced pressure to give a residue. The residue was purified by prep-HPLC (column: Waters Xbridge C18 150*50 mm*10 um; mobile phase: [water(NH₄HCO₃)-ACN]; B %: 42%-72%, 10 min) to give the compound tert-butyl N-[4-[[9-isopropyl-6-[[4-(2-pyridyl)phenyl]methylamino]purin-2-yl]amino]cyclohexyl]carbamate (360 mg, 30% yield) as a yellow oil.

Step 2. To a solution of tert-butyl N-[4-[[9-isopropyl-6-[[4-(2-pyridyl)phenyl]methylamino]purin-2-yl]amino]cyclohexyl]carbamate (90 mg, 161 umol, 1 equiv) in HCl/dioxane (1 mL) was added stirred at 20° C. for 1 h. The reaction mixture was filtered and concentrated under reduced pressure to give a residue. The crude product N₂-(4-amino-cyclohexyl)-9-isopropyl-N₆-[[4-(2-pyridyl)phenyl]methyl]purine-2,6-diamine (80 mg, crude, HCl) was used into the next step without further purification. Compound N₂-(4-amino-cyclohexyl)-9-isopropyl-N₆-[[4-(2-pyridyl)phenyl]methyl]purine-2,6-diamine (80 mg, crude, HCl) was obtained as a yellow solid.

### Compound AAC: 1-((4-amino-2-(2,6-dioxopiperidin-3-yl)-1-oxoisoindolin-5-yl)methyl)-3-(3-chloro-4-methylphenyl)urea

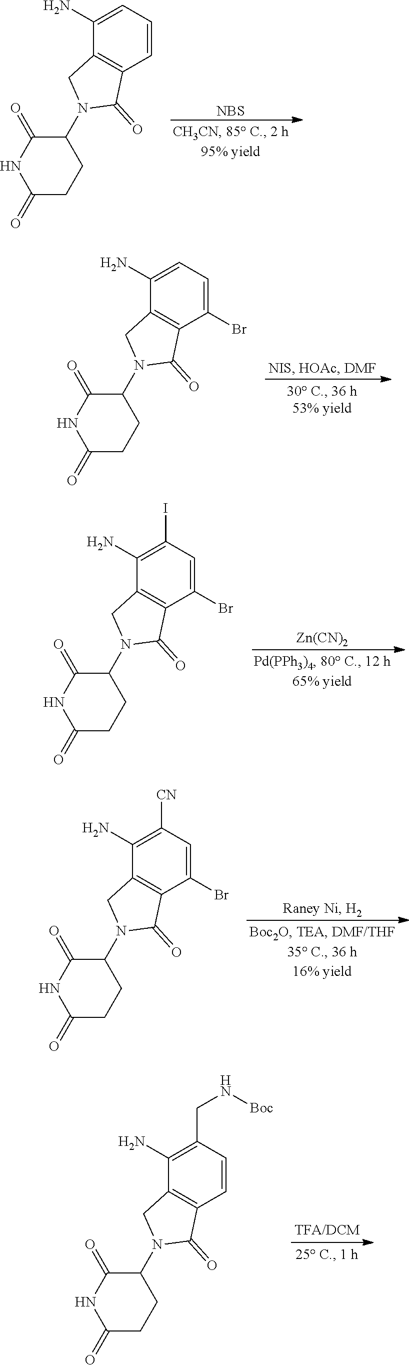

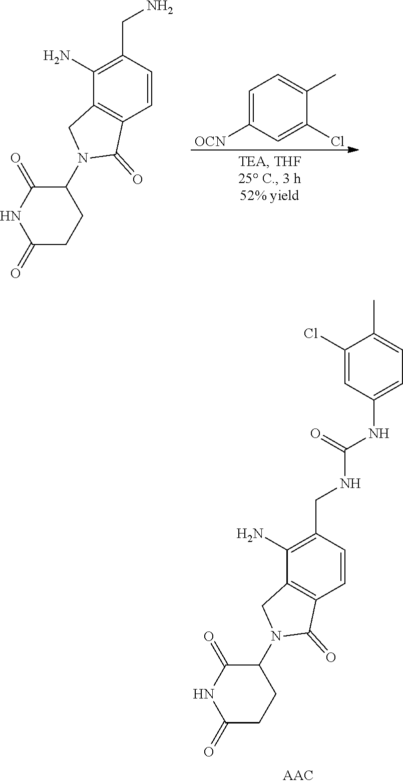

Step 1. A mixture of 3-(4-amino-1-oxo-isoindolin-2-yl)piperidine-2,6-dione (20.0 g, 77.1 mmol, 1.0 equiv), NBS (14.4 g, 81.0 mmol, 1.1 equiv) in CH₃CN (320 mL) was degassed and purged with N₂ for 3 times, and then the mixture was stirred at 85° C. for 2 h under N₂ atmosphere. The mixture was filtered to give the 3-(4-amino-7-bromo-1-oxo-isoindolin-2-yl)piperidine-2,6-dione (25.0 g, 73.9 mmol, 95% yield) was obtained as a brown solid ¹H NMR: (400 MHz, DMSO-d₆) δ=11.17-10.75 (m, 1H), 7.30 (d, J=8.4 Hz, 1H), 6.73 (d, J=8.4 Hz, 1H), 5.17-4.99 (m, 1H), 4.24-4.00 (m, 2H), 3.04-2.82 (m, 1H), 2.62 (d, J=17.2 Hz, 1H), 2.41-2.21 (m, 1H), 2.08-1.93 (m, 1H).

Step 2. A mixture of 3-(4-amino-7-bromo-1-oxo-isoindolin-2-yl)piperidine-2,6-dione (15.0 g, 44.4 mmol, 1.0 equiv), NIS (20.0 g, 88.7 mmol, 2.0 equiv) in HOAc (150 mL), DMF (150 mL) was degassed and purged with N₂ for 3 times, and then the mixture was stirred at 30° C. for 36 h under N₂ atmosphere. The reaction mixture was filtered and the filtrate was triturated with DMSO (100 mL, 80° C.) to give the 3-(4-amino-7-bromo-5-iodo-1-oxo-isoindolin-2-yl)piperidine-2,6-dione (11.0 g, 23.7 mmol, 53% yield) as a brown solid. ¹H NMR: (400 MHz, DMSO-d₆) δ 11.03 (s, 2H), 7.82 (s, 1H), 5.76 (s, 2H), 5.09 (d, J=5.2 Hz, 1H), 4.26-4.12 (m, 2H), 2.70-2.60 (m, 2H), 2.32-2.26 (m, 1H).

Step 3. A mixture of 3-(4-amino-7-bromo-5-iodo-1-oxo-isoindolin-2-yl)piperidine-2,6-dione (3.5 g, 7.5 mmol, 1.0 equiv), Zn(CN)₂ (885 mg, 7.5 mmol, 1.0 equiv), Pd(PPh₃)₄(871 mg, 754 umol, 0.1 equiv) in DMF (5 mL) was degassed and purged with N₂ for 3 times, and then the mixture was stirred at 80° C. for 12 h under N₂ atmosphere. The reaction mixture filtered and concentrated to get the residue. The residue was diluted with H₂O (10 mL) and extracted with DCM (10 mL×3). The combined organic layers were washed with brine (20 mL×3), dried over Na₂SO₄, filtered and concentrated under reduced pressure to give a residue. The solid was triturated with EA(30 ml) to 4-amino-7-bromo-2-(2,6-dioxo-3-piperidyl)-1-oxo-isoindoline-5-carbonitrile (1.8 g, 4.9 mmol, 65% yield) as a yellow solid. ¹H NMR: (400 MHz, DMSO-d₆) 11.05 (s, 1H), 7.95 (s, 1H), 6.58 (s, 2H), 5.08 (d, J=4.8, 13.1 Hz, 1H), 4.40-4.04 (m, 2H), 2.63 (d, J=17.2 Hz, 2H), 2.27 (d, J=4.0, 13.0 Hz, 1H), 2.16-2.01 (m, 1H).

Step 4. A mixture of 4-amino-7-bromo-2-(2,6-dioxo-3-piperidyl)-1-oxo-isoindoline-5-carbonitrile (1.7 g, 4.7 mmol, 1.0 equiv), Raney-Ni (401 mg, 4.7 mmol, 1.0 equiv), (Boc)₂O (5.1 g, 23.4 mmol, 5.4 mL, 5.0 equiv) and TEA (947 mg, 9.3 mmol, 1.3 mL, 2.0 equiv) in DMF (20 mL), THF (20 mL) degassed and purged with H₂ for 3 times, and then the mixture was stirred at 35° C. for 36 h. The mixture was filtered and concentrated to get the residue. The solid was triturated with EA (30 mL) to give the tert-butyl N-[[4-amino-2-(2,6-dioxo-3-piperidyl)-1-oxo-isoindolin-5-yl]methyl]carbamate (300 mg, 772 umol, 16% yield) as a yellow solid. ¹H NMR: (400 MHz, DMSO-d₆) 11.00 (s, 1H), 7.36 (t, J=6.0 Hz, 1H), 7.12 (d, J=7.6 Hz, 1H), 6.91 (d, J=7.6 Hz, 1H), 5.10 (d, J=5.2, 13.4 Hz, 1H), 4.24-4.06 (m, 4H), 3.05-2.77 (m, 2H), 2.70-2.61 (m, 2H), 2.37-2.24 (m, 1H), 2.06-1.96 (m, 1H), 1.53-1.28 (m, 9H).

Step 5. A mixture of tert-butyl N-[[4-amino-2-(2,6-dioxo-3-piperidyl)-1-oxo-isoindolin-5-yl]methyl]carbamate (70 mg, 180 umol, 1.0 equiv) in TFA (1 mL), DCM (3 mL) was degassed and purged with N₂ for 3 times, and then the mixture was stirred at 25° C. for 2 h under N₂ atmosphere. The mixture was concentrated to give the 3-[4-amino-5-(aminomethyl)-1-oxo-isoindolin-2-yl]piperidine-2,6-dione (70 mg, 174 umol, TFA) as a yellow oil.

Step 6. To a mixture of 3-[4-amino-5-(aminomethyl)-1-oxo-isoindolin-2-yl]piperidine-2,6-dione (50 mg, 125 umol, 1.0 equiv, TFA), TEA (13 mg, 125 umol, 1.0 equiv) THF (1 mL) stirred at 25° C., then the 2-chloro-4-isocyanato-1-methyl-benzene (25 mg, 135 umol, 1.1 equiv) was added to the mixture and stirred for 3 h under N₂ protection. The mixture was concentrated to get the residue. The residue was purified by the prep-HPLC(column: Phenomenex Luna C18 150*25 mm*10 um; mobile phase: [water(FA)-ACN]; B %: 24%-54%, 15 min) to give 1-[[4-amino-2-(2,6-dioxo-3-piperidyl)-1-oxo-isoindolin-5-yl]methyl]-3-(3-chloro-4-methyl-phenyl)urea (20 mg, 43 umol, 35% yield, 98% purity) as a yellow solid. ¹H NMR: (400 MHz, DMSO-d₆) 10.99 (s, 1H), 8.85 (s, 1H), 7.65 (d, J=2.0 Hz, 1H), 7.28-7.03 (m, 3H), 6.92 (d, J=7.6 Hz, 1H), 6.83-6.70 (m, 1H), 5.47 (s, 2H), 5.20-4.99 (m, 1H), 4.36-4.04 (m, 4H), 3.04-2.82 (m, 1H), 2.70-2.60 (m, 1H), 2.35-2.19 (m, 4H), 2.06-1.95 (m, 1H)

### Compound AAD: 1-(3-chloro-2-hydroxyphenyl)-3-((2-(2,6-dioxopiperidin-3-yl)-1-oxoisoindolin-5-yl)methyl)urea

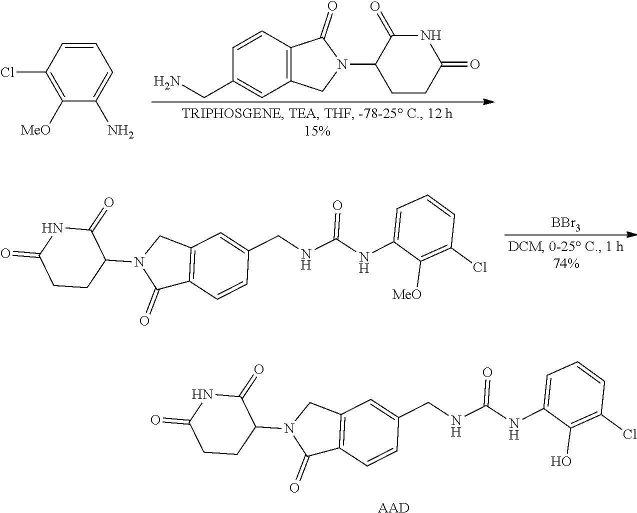

Step 1. To a solution of triphosgene (1.62 g, 5.45 mmol, 0.8 equiv) in THF (60 mL) was added 3-chloro-2-methoxy-aniline (1.00 g, 6.35 mmol, 1.0 equiv), TEA (6.42 g, 63.5 mmol, 9 mL, 10.0 equiv) in THF (20 mL) slowlyat −78° C., and then it was stirred for 0.5 h. 3-(5-(aminomethyl)-1-oxoisoindolin-2-yl)piperidine-2,6-dione (J. Am. Chem. Soc., 2017, 139, 15308) (1.97 g, 6.35 mmol, 1.0 equiv, HCl) was added to the mixture and then it was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Phenomenex luna C18 150*40 mm*15 um; mobile phase: [water(FA)-ACN]; B %: 27%-57%, 10 min). Compound 1-(3-chloro-2-methoxy-phenyl)-3-[[2-(2,6-dioxo-3-piperidyl)-1-oxo-isoindolin-5-yl]methyl]urea (440 mg, 953 umol, 15% yield) was obtained as a white solid.

Step 2. To a solution of 1-(3-chloro-2-methoxy-phenyl)-3-[[2-(2,6-dioxo-3-piperidyl)-1-oxo-isoindolin-5-yl]methyl]urea (440 mg, 963 umol, 1.0 equiv) in DCM (9 mL) was added BBr₃ (1.21 g, 4.82 mmol, 464 μL, 5.0 equiv) at 0° C. The mixture was stirred at 0-25° C. for 1 h. The reaction mixture was quenched by addition H₂O 20 mL at 0° C., and the solution was filtered, the filter residue was rinsed with 20 ml H₂O for 3 times, and the filter residue was dried in vacuum. The crude product was triturated with H₂O at 0° C. for 30 min and the solution was filtered, the filter residue was rinsed with 20 ml H₂O for 3 times, and the filter residue was dried in vacuum. Compound 1-(3-chloro-2-hydroxy-phenyl)-3-[[2-(2,6-dioxo-3-piperidyl)-1-oxo-isoindolin-5-yl]methyl]urea (315 mg, 711 umol, 74% yield) was obtained as a yellow solid. ¹H NMR: (DMSO-d₆, 400 MHz) 10.98 (s, 1H), 10.10-9.73 (m, 1H), 8.50-8.18 (m, 1H), 7.84-7.64 (m, 2H), 7.58-7.48 (m, 2H), 7.48-7.41 (m, 1H), 7.02-6.90 (m, 1H), 6.83-6.64 (m, 1H), 5.16-5.00 (m, 1H), 4.51-4.40 (m, 3H), 4.36-4.23 (m, 1H), 3.00-2.81 (m, 1H), 2.68-2.62 (m, 1H), 2.41-2.31 (m, 1H), 2.07-1.93 (m, 1H).

### Compound AAE: 1-((2-(2,6-dioxopiperidin-3-yl)-1-oxoisoindolin-5-yl)methyl)-3-(2-hydroxy-3-(trifluoromethyl)benzyl)urea

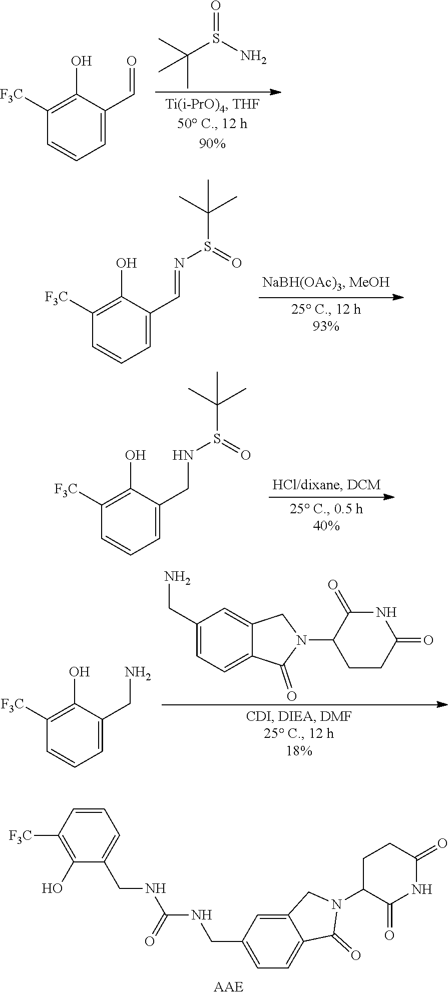

Step 1. To a solution of 2-methylpropane-2-sulfinamide (956 mg, 7.89 mmol, 1.0 equiv) in THF (10 mL) was added Ti(i-PrO)₄ (4.48 g, 15.78 mmol, 2.0 equiv) and 2-hydroxy-3-(trifluoromethyl)benzaldehyde (1.5 g, 7.89 mmol, 1.0 equiv). The mixture was stirred at 50° C. for 12 h and concentrated. The residue was purified by prep-HPLC (column: Welch Ultimate XB-CN 250*70*10 um; mobile phase:[Hexane-EtOH]; B %: 1%-30%, 15 min). to give (NE)-N-[[2-hydroxy-3-(trifluoromethyl)phenyl]methylene]-2-methyl-propane-2-sulfinamide (2.08 g, 90% yield)

Step 2. To a solution of (NE)-N-[[2-hydroxy-3-(trifluoromethyl)phenyl]methylene]-2-methyl-propane-2-sulfinamide (1.8 g, 6.14 mmol, 1.0 equiv) in MeOH (10 mL) was added NaBH(OAc)₃ (6.50 g, 30.68 mmol, 5.0 equiv). The mixture was stirred at 25° C. for 12 h. The mixture was Filtered and concentrated. The residue was purified by column chromatography (SiO₂, Petroleum ether/Ethyl acetate=10/1 to 0/1) to give N-[[2-hydroxy-3-(trifluoromethyl)phenyl]methyl]-2-methyl-propane-2-sulfinamide (1.68 g, 93% yield)

Step 3. To a solution of N-[[2-hydroxy-3-(trifluoromethyl)phenyl]methyl]-2-methyl-propane-2-sulfinamide (1 g, 3.39 mmol, 1.0 equiv) in DCM (10 mL) was added HCl/dioxane (4 M, 10.00 mL, 11.8 equiv). The mixture was stirred at 25° C. for 0.5 h. The mixture was concentrated. The residue was purified by prep-HPLC (column: Waters Xbridge C18 150*50 mm*10 um; mobile phase: [water(NH₄HCO₃)-ACN]; B %: 9%-39%, 10 min). to give 2-(aminomethyl)-6-(trifluoromethyl)phenol (260 mg, 40% yield).

Step 4. To a solution of 3-[5-(aminomethyl)-1-oxo-isoindolin-2-yl]piperidine-2,6-dione (353.82 mg, 1.14 mmol, 1.0 equiv, HCl) in DMF (10 mL) was added CDI (277.84 mg, 1.71 mmol, 1.5 equiv) and DIEA (738.15 mg, 5.71 mmol, 995 μL, 5.0 equiv) The mixture was stirred at 25° C. for 1 h. To a solution of 2-(aminomethyl)-6-(trifluoromethyl)phenol (260 mg, 1.14 mmol, 1.0 equiv, HCl) in DMF (10 mL) was added DIEA (738.15 mg, 5.71 mmol, 995 μL, 5.0 equiv)CDI (277.84 mg, 1.71 mmol, 1.5 equiv). The reaction mixture was concentrated. The residue was purified by prep-HPLC (column: Unisil 3-100 C18 Ultra 150*50 mm*3 um; mobile phase: [water(FA)-ACN]; B %: 23%-53%, 7 min). to give 1-[[2-(2,6-dioxo-3-piperidyl)-1-oxo-isoindolin-5-yl]methyl]-3-[[2-hydroxy-3-(trifluoromethyl)phenyl]methyl]urea (109 mg, 18% yield). ¹H NMR (400 MHz, DMSO+D2O) δ 7.67 (d, J=7.6 Hz, 1H), 7.53-7.33 (m, 4H), 6.92 (s, 1H), 5.05 (s, 1H), 4.46-4.26 (m, 4H), 4.19 (s, 2H), 2.92-2.81 (m, 1H), 2.01 (s, 1H).

### Compound AAF: 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-2-(cyanomethyl)piperazine-1-carbonyl)allyl 4-methylbenzenesulfonate

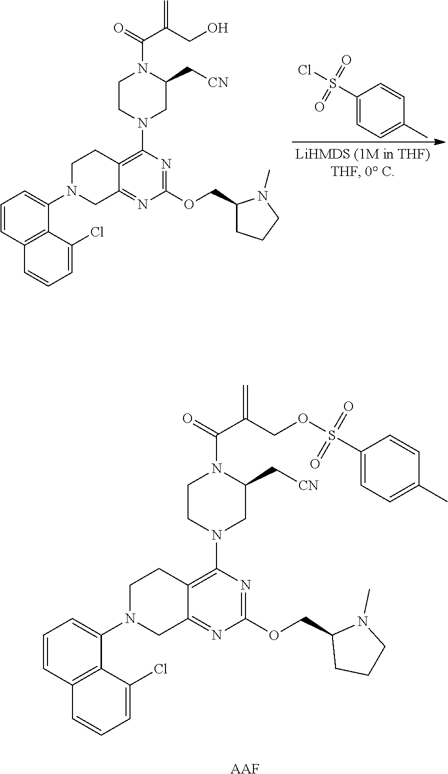 [SMILES: C=C(CO)C(=O)N1CCN(c2nc(OC[C@@H]3CCCN3C)nc3c2CCN(c2cccc4cccc(Cl)c24)C3)C[C@@H]1CC#N ; Cc1ccc(S(=O)(=O)Cl)cc1 ; C=C(COS(=O)(=O)c1ccc(C)cc1)C(=O)N1CCN(c2nc(OC[C@@H]3CCCN3C)nc3c2CCN(c2cccc4cccc(Cl)c24)C3)C[C@@H]1CC#N ; "AAF"]

Step 1. To a solution of 2-((S)-4-(7-(8-chloronaphthalen-1-yl)-2-(((S)-1-methylpyrrolidin-2-yl)methoxy)-5,6,7,8-tetrahydropyrido [3,4-d]pyrimidin-4-yl)-1-(2-(hydroxymethyl)acryloyl) piperazin-2-yl)acetonitrile (20.0 mg, 32.4 μmol) and tosyl chloride (6.8 mg, 36 μmol) in dry THF (1.5 mL) containing several beads of 4 A molecular sieves was added LiHMDS (1 M in THF, 39 μL, 39 μmol) at 0° C. under nitrogen. The reaction mixture was stirred at 0° C. for 1 h. Additional tosyl chloride (6.8 mg, 36 μmol) and LiHMDS (1 M in THF, 39 μL, 39 μmol) were sequentially added and the reaction mixture was stirred at 0° C. for 2 h. The volatiles were removed under reduced pressure, and the residual material was diluted with hexanes (2.0 mL) and sonicated for 5 min. After settling, the liquid was decanted, and the residual material was dried under vacuum and used in the next step without further purification.

### Compound AAG: 1-((2-(2,6-dioxopiperidin-3-yl)-1-oxoisoindolin-5-yl)methyl)-3-(2-hydroxy-5-methoxybenzyl)urea

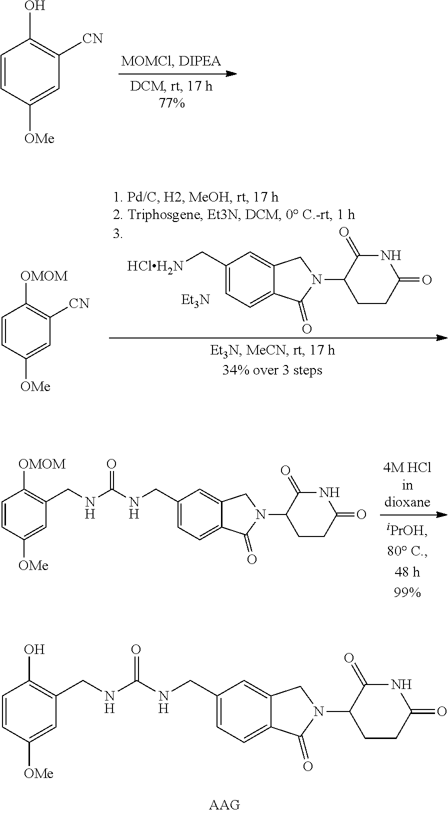

Step 1. To a solution of 2-hydroxy-5-methoxybenzonitrile 1 (1.00 g, 6.70 mmol) and DIPEA (2.35 mL, 13.4 mmol) in DCM (50 mL) was added chloromethylmethylether (1.02 mL, 13.4 mmol) at rt under nitrogen. The reaction mixture was stirred at rt for 17 h and then diluted with water (20 mL). The organic phase was separated, and the aqueous phase was extracted with DCM (3×20 mL). The combined organic phases were washed with water (2×20 mL) and brine (10 mL), dried over MgSO₄, filtered, and concentrated under reduced pressure. The crude product was purified by column chromatography on silica gel using a gradient of 0-100% EtOAc in hexane to provide 5-methoxy-2-(methoxymethoxy)benzonitrile(995 mg, 77%) as a solid. ¹H NMR (DMSO-d₆, 400 MHz): δ_{H} 7.33 (1H, d, J=2.9 Hz), 7.28-7.22 (2H, m), 5.28 (2H, s), 3.75 (3H, s), 3.42 (3H, s).

Step 2. 1) A mixture of 5-methoxy-2-(methoxymethoxy)benzonitrile (809 mg, 4.19 mmol) and 10 wt % Pd/C (356 mg, 0.335 mmol) in MeOH (90 mL) was subjected to hydrogenation at 1 atm and rt for 17 h. The reaction mixture was filtered through Celite and the filtrate was concentrated under reduced pressure to provide the crude benzylamine as an oil. This intermediate was used in the next step without further purification.

Step 2: 2) A solution of the benzylamine intermediate obtained from step 1 in DCM (10 mL) was added to a solution of triphosgene (869 mg, 2.93 mmol) in DCM (30 mL) at rt under nitrogen. The mixture was cooled to 0° C. and a solution of Et₃N (0.874 mL, 6.27 mmol) in DCM (4.0 mL) was added. The reaction mixture was stirred at rt for 1 h and then concentrated under reduced pressure to afford the crude isocyanate which was used in the next step without further purification.

Step 2: 3) To a solution of the isocyanate intermediate obtained from step 2 and 3-(5-(aminomethyl)-1-oxoisoindolin-2-yl)piperidine-2,6-dione hydrochloride 3 (908 mg, 2.93 mmol) in MeCN (30 mL) was added Et₃N (0.817 mL, 5.86 mmol) at rt under nitrogen. The reaction mixture was stirred at rt for 17 h. Water (30 mL), EtOAc (35 mL) and an aqueous solution of HCl (1 M, 10 mL) were sequentially added. The organic phase was separated, and the aqueous phase was extracted with EtOAc (2×25 mL). The combined organic phases were dried over MgSO₄, filtered, and concentrated under reduced pressure. The crude product was purified by column chromatography on silica gel using a gradient of 0-100% isopropyl alcohol in CHCl₃ to provide 1-((2-(2,6-dioxopiperidin-3-yl)-1-oxoisoindolin-5-yl)methyl)-3-(5-methoxy-2-(methoxymethoxy)benzyl)urea (440 mg, 30%) as a solid.

Step 3. A mixture of 1-((2-(2,6-dioxopiperidin-3-yl)-1-oxoisoindolin-5-yl)methyl)-3-(5-methoxy-2-(methoxymethoxy)benzyl)urea (329 mg, 0.663 mmol) and a solution of HCl in dioxane (4 M, 8.28 mL, 33.1 mmol) in a mixture of dioxane (50 mL) and ⁱPrOH (4.0 mL) was stirred at 80° C. for 48 h and then cooled to rt. The volatiles were evaporated under reduced pressure and the residual material was lyophilized to provide 1-((2-(2,6-dioxopiperidin-3-yl)-1-oxoisoindolin-5-yl)methyl)-3-(2-hydroxy-5-methoxybenzyl)urea as a solid (298 mg, 99%). The crude phenol was used in the next step without further purification. Note: a small fraction was purified by semi-preparative HPLC (Gemini® 5 μm NX-C18 110 Å, 100×30 mm) using a gradient of 40-100% MeCN in 10 mM ammonium formate to provide 1-((2-(2,6-dioxopiperidin-3-yl)-1-oxoisoindolin-5-yl)methyl)-3-(2-hydroxy-5-methoxybenzyl)urea with a 99% purity. ¹H NMR (DMSO-d₆, 400 MHz): δ_{H} 2.01-1.96 (1H, m), 2.43-2.31 (1H, m), 2.68-2.58 (1H, m), 2.95-2.86 (1H, m), 3.63 (3H, s), 4.12 (2H, d, J=6.0 Hz), 4.36-4.28 (3H, m), 4.42 (1H, d, J=17.2 Hz), 5.10 (1H, dd, J=13.3, 5.1 Hz), 6.51 (1H, t, J=6.0 Hz), 6.71-6.63 (3H, m), 6.76 (1H, t, J=6.1 Hz), 7.39 (1H, d, J=7.9 Hz), 7.46 (1H, s), 7.66 (1H, d, J=7.8 Hz), 9.28 (1H, s), 10.93 (1H, br s).

### Compound AAH: tert-butyl 2-((6-(3-((2-(2,6-dioxopiperidin-3-yl)-1-oxoisoindolin-5-yl)methyl)ureido)-1H-indol-1-yl)methyl)acrylate

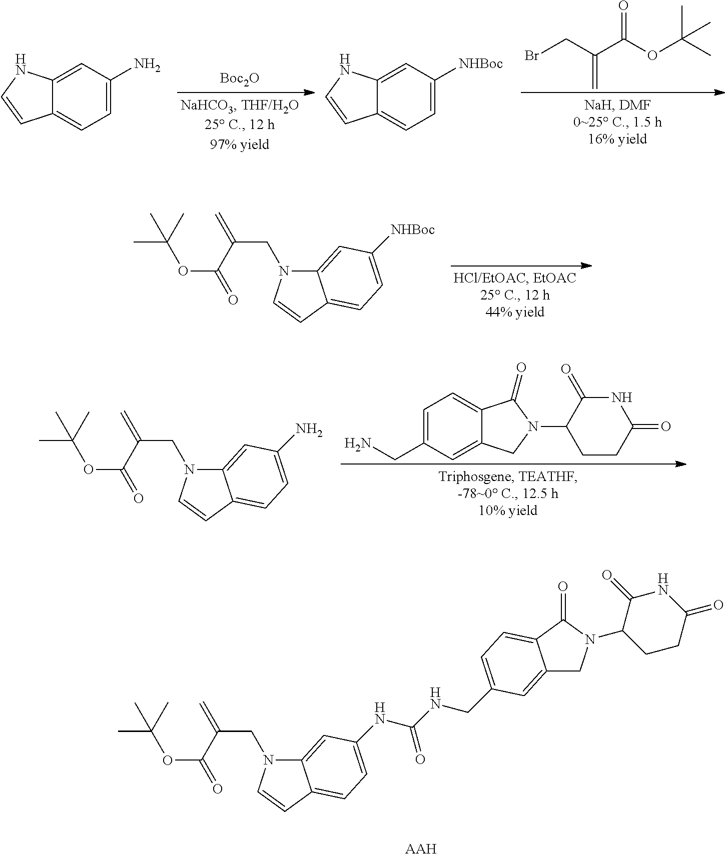

Step 1. To a solution of 1H-indol-6-amine (3.00 g, 22.7 mmol, 1.0 equiv) in THF (10 mL) was added Boc₂O (9.91 g, 45.4 mmol, 10.4 mL, 2.0 equiv) and saturated NaHCO₃ (10.0 mL) solution. The mixture was stirred at 25° C. for 12 h. The resultant mixture was diluted with water (30 mL) and the aqueous phase was extracted with ethyl acetate (30 mL×2). The combined organic phase was washed with brine (30 mL×2), dried over anhydrous sodium sulfate, filtered and concentrated under vacuum to give a residue. The residue was purified by flash silica gel chromatography (ISCO®; 20 g SepaFlash® Silica Flash Column, Eluent of 0-18% Ethyl acetate/Petroleum ethergradient@ 50 mL/min) to afford the product tert-butyl N-(1H-indol-6-yl)carbamate (5.10 g, 22.0 mmol, 97% yield) as a red solid. ¹H NMR (400 MHz, DMSO-d6) δ=10.87 (br s, 1H), 9.14 (br s, 1H), 7.68 (br s, 1H), 7.35 (br d, J=8.4 Hz, 1H), 7.22-7.17 (m, 1H), 6.98 (br d, J=8.4 Hz, 1H), 6.30 (br d, J=0.8 Hz, 1H), 1.48 (s, 9H)

Step 2. To a mixture of tert-butyl 2-(bromomethyl)prop-2-enoate (WO2011/25927, 2011, A1) (1.90 g, 8.61 mmol, 1.0 equiv) in THF (40.0 mL) was added NaH (517 mg, 12.9 mmol, 60% purity, 1.5 equiv) and stirred at 0° C. for 0.5 h. Then tert-butyl N-(1H-indol-6-yl)carbamate (2.00 g, 8.61 mmol, 1.0 equiv) was added to the mixture and the mixture was stirred at 25° C. for 1 h. The reaction mixture was quenched with saturated aqueous solution of NH₄Cl (10 mL) at 0° C. The resultant mixture was diluted with water (20 mL) and the aqueous phase was extracted with ethyl acetate (30 mL×2). The combined organic phase was washed with brine (20 mL×2), dried over anhydrous sodium sulfate, filtered and concentrated under vacuum to give a residue. The residue was purified by prep-HPLC (FA condition; column: YMC Triart C18 250*50 mm*7 um; mobile phase:[water(FA)-ACN]; B %:80%-90%,22 min) to afford the desired product tert-butyl 2-[[6-(tert-butoxycarbonylamino)indol-1-yl]methyl]prop-2-enoate (500 mg, 1.34 mmol, 16% yield) as a white solid. ¹H NMR (400 MHz, DMSO-d6) δ=9.20 (br s, 1H), 7.59 (br s, 1H), 7.39 (d, J=8.4 Hz, 1H), 7.21 (d, J=2.0 Hz, 1H), 7.01 (br d, J=8.0 Hz, 1H), 6.37 (br s, 1H), 6.05 (s, 1H), 5.09 (br s, 1H), 4.90 (s, 2H), 1.47 (s, 9H), 1.44 (s, 9H)

Step 3. To a solution of tert-butyl 2-[[6-(tert-butoxycarbonylamino)indol-1-yl]methyl]prop-2-enoate (200 mg, 537 umol, 1.0 equiv) in EtOAc (9.0 mL) was added HCl/EtOAc (4N, 3.0 mL). The mixture was stirred at 25° C. for 12 h. The resultant mixture was diluted with water (20 mL) and the aqueous phase was extracted with ethyl acetate (30 mL×2). The combined organic phase was washed with brine (20 mL×2), dried over anhydrous sodium sulfate, filtered and concentrated under vacuum to give a residue. The residue was purified by prep-TLC (SiO₂, Petroleum ether: Ethyl acetate=3:1) to give the desired product tert-butyl 2-[(6-aminoindol-1-yl)methyl]prop-2-enoate (65.0 mg, 239 umol, 44% yield) as a red solid. ¹H NMR (400 MHz, DMSO-d6) δ=7.18 (d, J=8.4 Hz, 1H), 6.97 (d, J=3.2 Hz, 1H), 6.50-6.34 (m, 2H), 6.22 (d, J=3.2 Hz, 1H), 6.02 (s, 1H), 5.03 (s, 1H), 4.82-4.70 (m, 4H), 1.45 (s, 9H)

Step 4. To a solution of triphosgene (70.0 mg, 235 umol, 1.1 equiv) in THF (3 mL) was drop-wise added tert-butyl 2-[(6-aminoindol-1-yl)methyl]prop-2-enoate (60.0 mg, 220 umol, 1.0 equiv) and TEA (178 mg, 1.76 mmol, 245 μL, 8.0 equiv) in THF (1 mL) at −78° C. The mixture was stirred at −78° C. for 0.5 h. 3-[5-(aminomethyl)-1-oxo-isoindolin-2-yl]piperidine-2,6-dione (68.2 mg, 220.3 umol, 1.0 equiv, HCl) was added into the reaction mixture and stirred at 20° C. for 12 h. The resultant mixture was diluted with water (20 mL) and NaHCO₃ (10 mL). The solution was extracted with ethyl acetate (30 mL×2). The combined organic phase was washed with brine (30 mL×2), dried over anhydrous sodium sulfate, filtered and concentrated under vacuum to give a residue. The residue was purified by prep-HPLC (FA condition; column: Phenomenex luna C18 150*25 mm*10 um; mobile phase: [water(FA)-ACN]; B %: 38%-68%, 10 min) to give tert-butyl 2-[[6-[[2-(2,6-dioxo-3-piperidyl)-1-oxo-isoindolin-5-yl]methylcarbamoylamino]indol-1-yl]methyl]prop-2-enoate (13.4 mg, 21.9 umol, 10% yield, 93.2% purity) as a white solid. ¹H NMR (400 MHz, CD₃OD-d4) δ=7.78 (d, J=8.0 Hz, 1H), 7.59-7.54 (m, 2H), 7.51 (d, J=8.4 Hz, 1H), 7.45 (d, J=8.4 Hz, 1H), 7.12 (d, J=3.2 Hz, 1H), 6.89 (dd, J=1.6, 8.4 Hz, 1H), 6.41 (d, J=2.4 Hz, 1H), 6.11 (d, J=0.8 Hz, 1H), 5.20-5.11 (m, 2H), 4.94 (s, 2H), 4.53 (s, 2H), 4.49 (d, J=7.2 Hz, 2H), 2.96-2.85 (m, 1H), 2.83-2.74 (m, 1H), 2.55-2.43 (m, 1H), 2.23-2.11 (m, 1H), 1.46 (s, 9H).

### Compound AAI: 2-((4-(8-bromo-4-(((5-methyl-1H-benzo[d]imidazol-2-yl)methyl)amino)pyrazolo[1,5-a][1,3,5]triazin-2-yl)piperazin-1-yl)methyl)acrylic acid

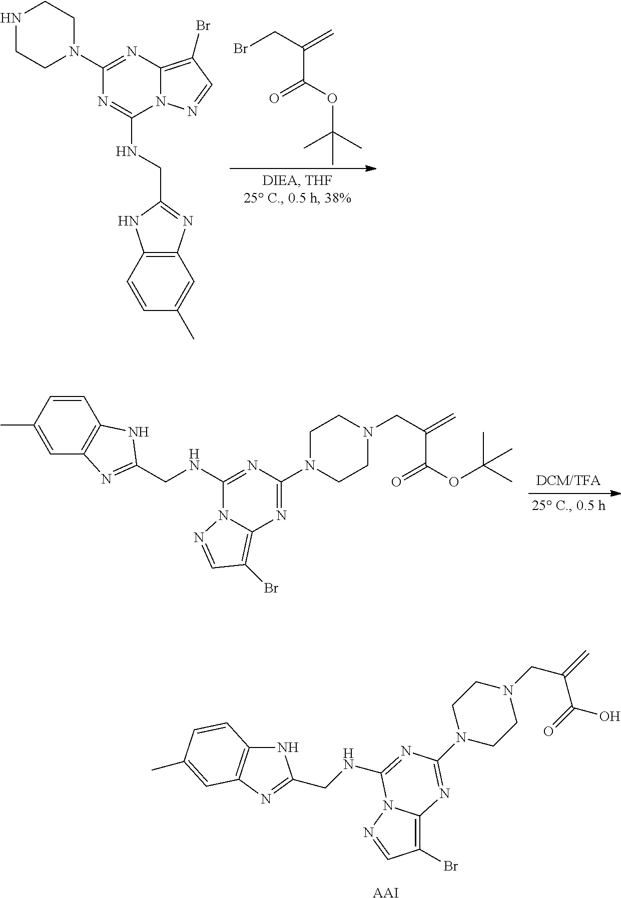

Step 1. To a solution of 8-bromo-N-[(5-methyl-1H-benzimidazol-2-yl)methyl]-2-piperazin-1-yl-pyrazolo[1,5-a][1,3,5]triazin-4-amine (WO2021/116178, 2021, AI) (500.0 mg, 898.7 umol, 1.0 equiv, TFA salt), tert-butyl 2-(bromomethyl)prop-2-enoate (198.7 mg, 898.7 umol, 1.0 equiv) in THF (2.0 mL) was added DIEA (348.4 mg, 2.7 mmol, 469.6 uL, 3.0 equiv). The mixture was stirred at 25° C. for 0.5 h. The residue was purified by prep-HPLC (column: Waters Xbridge C18 150*50 mm*10 um; mobile phase: [water (NH₄HCO₃)-ACN]; B %: 54%-84%, 10 min) to give compound tert-butyl 2-[[4-[8-bromo-4-[(5-methyl-1H-benzimidazol-2-yl)methylamino]pyrazolo[1,5-a][1,3,5]triazin-2-yl]piperazin-1-yl]methyl]prop-2-enoate (0.2 g, 38% yield) as a white solid.

Step 2. To a solution of tert-butyl 2-[[4-[8-bromo-4-[(5-methyl-1H-benzimidazol-2-yl)methylamino]pyrazolo[1,5-a][1,3,5]triazin-2-yl]piperazin-1-yl]methyl]prop-2-enoate (100.0 mg, 171.7 umol, 1.0 equiv) in DCM (1.0 mL) was added TFA (0.5 mL), and then it was stirred at 25° C. for 0.5 h. The reaction mixture was concentrated under reduced pressure to give a compound 2-[[4-[8-bromo-4-[(5-methyl-H-benzimidazol-2-yl)methylamino]pyrazolo[1,5-a][1,3,5]triazin-2-yl]piperazin-1-yl]methyl]prop-2-enoic acid (100 mg, 82% yield, TFA salt) as a colorless oil.

### Compound AAJ: 1-((2-(2,6-dioxopiperidin-3-yl)-1-oxoisoindolin-5-yl)methyl)-3-(2-hydroxyphenyl)urea

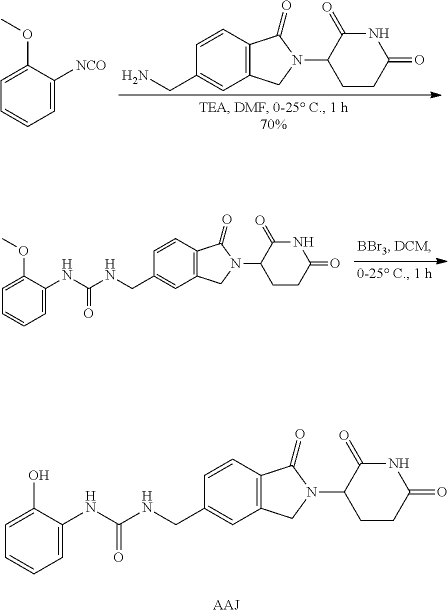

Step 1. To a solution of compound 2 (J. Am. Chem. Soc., 2017, 139, 15308) (740 mg, 1.67 mmol, 70% purity, 1.0 equiv, HCl salt) in DMF (4 mL) was added TEA (507 mg, 5.02 mmol, 698 μL, 3.0 equiv) dropwise and 1-isocyanato-2-methoxybenzene (498 mg, 3.34 mmol, 445 μL, 2.0 equiv) at 0° C. After addition, the mixture was stirred at 25° C. for 1 h. The reaction mixture was quenched by addition H₂O (50 mL), and then extracted with EtOAc (2×30 mL). The combined organic layers were washed with brine (10 mL), dried over Na₂SO₄, filtered and concentrated under reduced pressure to give a residue. The residue was purified by column chromatography (SiO₂, Petroleum ether/Ethyl acetate=1/1 to EtOAc: MeOH=10:1). 1-((2-(2,6-dioxopiperidin-3-yl)-1-oxoisoindolin-5-yl)methyl)-3-(2-methoxyphenyl)urea(500 mg, 1.18 mmol, 70% yield) was obtained as a white solid.

Step 2. To a solution of 1-((2-(2,6-dioxopiperidin-3-yl)-1-oxoisoindolin-5-yl)methyl)-3-(2-methoxyphenyl)urea (490 mg, 1.16 mmol, 1.0 equiv) in DCM (5 mL) was added BBr₃ (1.45 g, 5.80 mmol, 558 μL, 5.0 equiv) at 0° C. The mixture was stirred at 25° C. for 1 h. The reaction mixture was quenched by addition H₂O (5 mL) at 0° C., and then filtered to give the crude product 1-((2-(2,6-dioxopiperidin-3-yl)-1-oxoisoindolin-5-yl)methyl)-3-(2-hydroxyphenyl)urea (450 mg) was as a yellow solid.

### Compound AAK: 2-(4-chloro-3-hydroxyphenyl)-N-((2-(2,6-dioxopiperidin-3-yl)-1-oxoisoindolin-5-yl)methyl)-2,2-difluoroacetamide

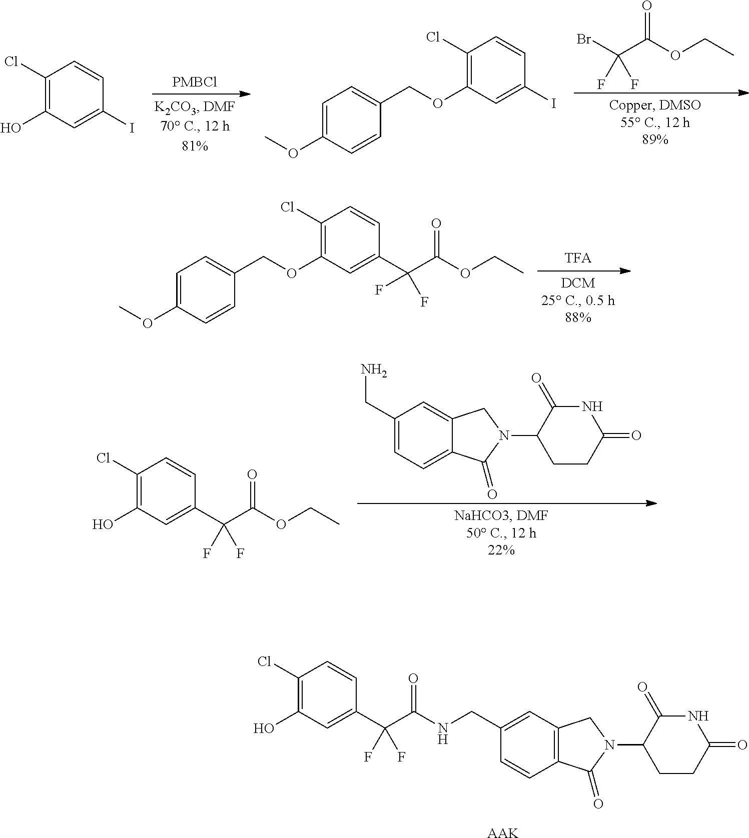

Step 1. To a solution of PMB-Cl (369 mg, 2.36 mmol, 1.2 equiv) and 2-chloro-5-iodo-phenol (500 mg, 1.97 mmol, 1.0 equiv) in DMF (10 mL) was added K2CO3 (543 mg, 3.93 mmol, 2.0 equiv). The mixture was stirred at 70° C. for 12 h. The mixture was concentrated to give a residue. The residue was purified by silica column chromatography on silica gel (Petroleum ether: Ethyl acetate from 50/1 to 20/1) to give compound 1-chloro-4-iodo-2-[(4-methoxyphenyl)methoxy]benzene (600 mg, 81% yield). ¹H NMR (400 MHz, CDCl₃): δ 7.56 (d, J=1.6 Hz, 1H), 7.40 (d, J=8.6 Hz, 2H), 7.32 (d, J=1.7, 8.3 Hz, 1H), 7.26-7.19 (m, 1H), 6.98 (d, J=8.6 Hz, 2H), 5.14 (s, 2H), 3.77 (s, 3H).

Step 2. To a solution of 1-chloro-4-iodo-2-[(4-methoxyphenyl)methoxy]benzene (4.5 g, 12 mmol, 1.0 equiv) in DMSO (30 mL) was added copper (3.8 g, 60 mmol, 5.0 equiv) and ethyl 2-bromo-2,2-difluoro-acetate (3.9 g, 19 mmol, 1.6 equiv). The mixture was stirred at 55° C. for 12 h. The reaction mixture was filtered and concentrated under reduced pressure to give a residue. The residue was purified by silica column chromatography on silica gel (Petroleum ether: Ethyl acetate from 50/1 to 20/1) to give ethyl 2-[4-chloro-3-[(4-methoxyphenyl)methoxy]phenyl]-2,2-difluoro-acetate (4 g, 89% yield). ¹H NMR (400 MHz, CDCl₃): δ 7.48 (d, J=8.2 Hz, 1H), 7.42 (d, J=8.6 Hz, 2H), 7.28 (s, 1H), 7.18-7.15 (m, 1H), 6.96 (d, J=8.7 Hz, 2H), 5.13 (s, 2H), 4.30 (q, J=7.1 Hz, 2H), 3.84 (s, 3H), 1.31 (t, J=7.2 Hz, 3H).

Step 3. To a solution of ethyl 2-[4-chloro-3-[(4-methoxyphenyl)methoxy]phenyl]-2,2-difluoro-acetate (1 g, 2.70 mmol, 1.0 equiv) in DCM (5 mL) was added TFA (4.6 g, 40 mmol, 3 mL, 15 equiv). The mixture was stirred at 25° C. for 0.5 h. The reaction mixture filtered and concentrated under reduced pressure to give a residue. The residue was purified by prep-TLC (SiO2, PE:EA=5:1) to give ethyl 2-(4-chloro-3-hydroxy-phenyl)-2,2-difluoro-acetate (600 mg, 88% yield). ¹H NMR (400 MHz, CDCl₃): δ 7.43 (d, J=8.4 Hz, 1H), 7.35-7.25 (m, 1H), 7.15-7.13 (m, 1H), 4.33 (q, J=7.1 Hz, 2H), 1.33-1.25 (m, 3H).

Step 4. To a solution of ethyl 2-(4-chloro-3-hydroxy-phenyl)-2,2-difluoro-acetate (500 mg, 2.00 mmol, 1.0 equiv) and 3-[5-(aminomethyl)-1-oxo-isoindolin-2-yl] piperidine-2,6-dione (WO2021/198965, 2021, A1)(617 mg, 2.00 mmol, 1.0 equiv, HCl) in DMF (10 mL) was added NaHCO₃ (502 mg, 5.99 mmol, 232 μL, 3.0 equiv). The mixture was stirred at 50° C. for 12 h. The reaction mixture was filtered and concentrated under reduced pressure to give a residue. The residue was purified by prep-HPLC (column: Waters Xbridge C18 150*50 mm*10 um; mobile phase: [water(NH₄HCO₃)-ACN]; B %: 15%-45%, 10 min) to give compound 2-(4-chloro-3-hydroxy-phenyl)-N-[[2-(2,6-dioxo-3-piperidyl)-1-oxo-isoindolin-5-yl]methyl]-2,2-difluoro-acetamide (230 mg, 22% yield). ¹H NMR (400 MHz, DMSO): δ 9.63 (t, J=6.0 Hz, 1H), 7.69 (d, J=7.8 Hz, 1H), 7.50 (d, J=8.3 Hz, 1H), 7.41-7.35 (m, 2H), 7.18 (d, J=2.0 Hz, 1H), 7.04-6.94 (m, 1H), 5.13-5.08 (m, 1H), 4.59-4.32 (m, 4H), 4.32-4.24 (m, 1H), 3.01-2.84 (m, 1H), 2.43-2.29 (m, 2H), 2.17-1.92 (m, 2H).

### Compound AAL: 1-(5-chloro-2-hydroxy-4-nitrophenyl)-3-((2-(2,6-dioxopiperidin-3-yl)-1-oxoisoindolin-5-yl)methyl)urea

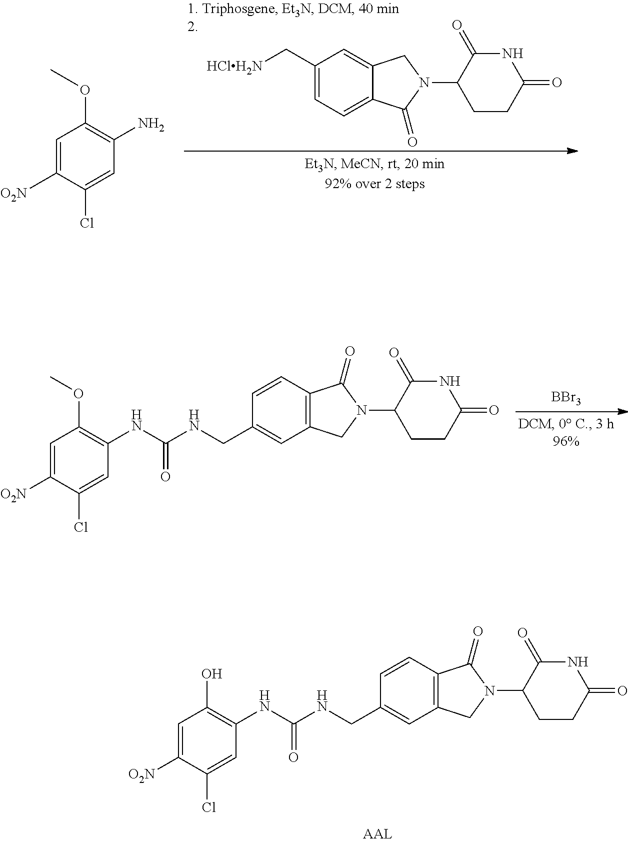

Step 1. A solution of 5-chloro-2-methoxy-4-nitroaniline 1 (955 mg, 4.71 mmol) in DCM (5.0 mL) was added to a mixture of triphosgene (1.40 g, 4.71 mmol) in DCM (10 mL) at rt under nitrogen. The mixture was cooled to 0° C. and a solution of Et₃N (1.12 mL, 8.01 mmol) in DCM (5.0 mL) was added. The mixture was stirred at rt for 40 min and then concentrated under reduced pressure. The residual material was dissolved in MeCN (35 mL) and 3-(5-(aminomethyl)-1-oxoisoindolin-2-yl)piperidine-2,6-dione, hydrochloride 2 (1.46 g, 4.71 mmol) and Et₃N (1.32 mL, 9.42 mmol) were sequentially added at rt under nitrogen. The reaction mixture was stirred at rt for 20 min and then diluted with an aqueous solution of HCl (1 M, 10 mL), EtOAc (30 mL) and water (40 mL). The organic phase was separated, and the aqueous phase was extracted with EtOAc (3×25 mL). The combined organic phases were washed with water (2×25 mL) and brine (15 mL), dried over MgSO₄, filtered, and concentrated under reduced pressure to provide the title compound (2.17 g, 92% over 2 steps) as a solid. ¹H NMR (DMSO-d₆, 400 MHz): δ_{H} 10.97 (1H, s), 8.78 (1H, s), 8.50 (1H, s), 7.78-7.75 (1H, m), 7.72-7.70 (2H, m), 7.53 (1H, s), 7.45 (1H, d, J=7.9 Hz), 5.10 (1H, dd, J=13.3, 5.0 Hz), 4.47-4.43 (3H, m), 4.32 (1H, d, J 17.4 Hz), 3.97 (3H, s), 2.95-2.86 (1H, m), 2.62-2.58 (1H, m), 2.43-2.33 (1H, m), 2.01-1.98 (1H, m).

Step 3. To a solution of 1-(5-chloro-2-methoxy-4-nitrophenyl)-3-((2-(2,6-dioxopiperidin-3-yl)-1-oxoisoindolin-5-yl)methyl)urea HD-E04-N007 (250 mg, 0.498 mmol) in DCM (50 mL) was added a solution BBr₃ in DCM (1 M, 9.27 mL, 9.27 mmol) at 0° C. under nitrogen. The mixture was stirred at rt for 3 h and then cooled to 0° C. Water (20 mL) was added at 0° C. and the volatiles were then removed under reduced pressure. The residual material was suspended in Et₂O (4.0 mL), centrifuged and the supernatant was separated. This process was repeated with MeOH (4.0 mL) and water (4.0 mL). The residual material was suspended in water and lyophilized to 1-(5-chloro-2-hydroxy-4-nitrophenyl)-3-((2-(2,6-dioxopiperidin-3-yl)-1-oxoisoindolin-5-yl)methyl)urea (234 mg, 96%) as a solid. ¹H NMR (DMSO-d₆, 400 MHz) δ_{H} 11.20 (s, 1H), 10.98 (s, 1H), 8.71 (s, 1H), 8.43 (s, 1H), 7.82-7.75 (m, 1H), 7.70 (d, J=7.2 Hz, 1H), 7.57-7.50 (m, 2H), 7.45 (d, J=7.2 Hz, 1H), 5.10 (dd, J=13.8, 5.9 Hz, 1H), 4.52-4.40 (m, 3H), 4.31 (d, J=17.2 Hz, 1H), 2.97-2.84 (m, 1H), 2.64-2.55 (m, 1H), 2.44-2.30 (m, 1H), 2.07-1.93 (m, 1H).

### Compound AAM: 2-((5-chloro-3-(3-((2-(2,6-dioxopiperidin-3-yl)-1-oxoisoindolin-5-yl)methyl)ureido)-2-fluorophenoxy)methyl)acrylic acid

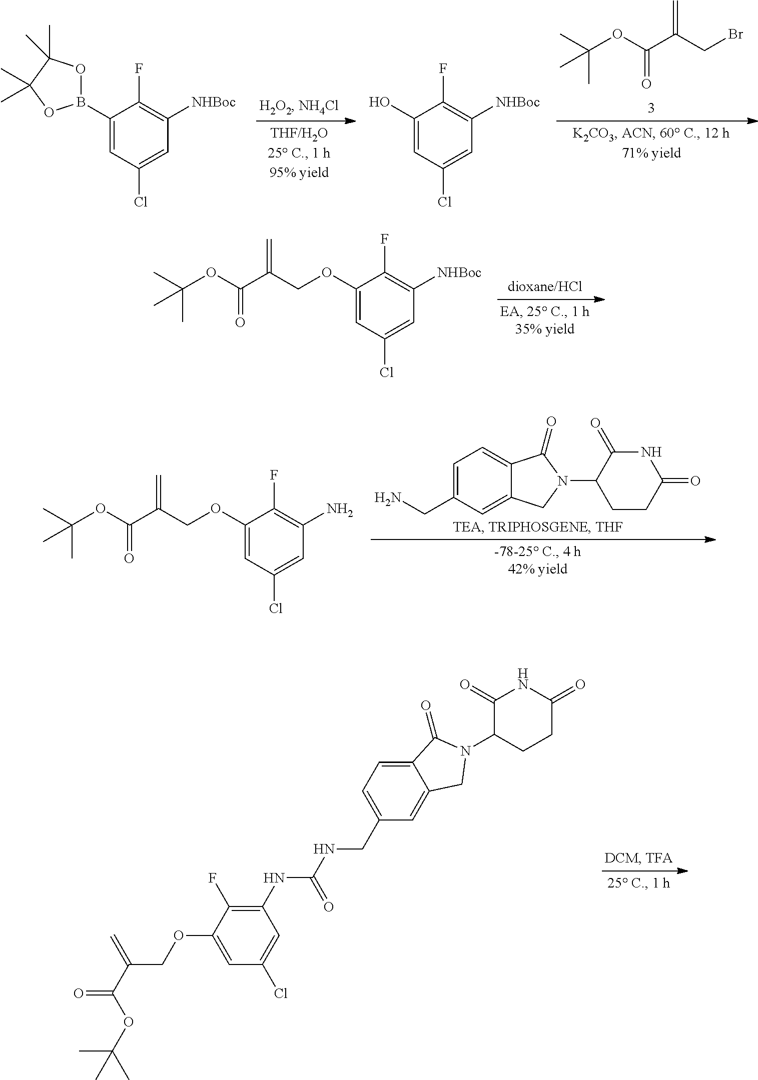

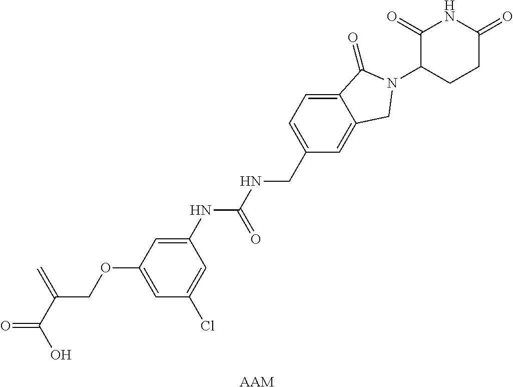

Step 1. A mixture of tert-butyl N-[5-chloro-2-fluoro-3-(4,4,5,5-tetramethyl-1,3,2-dioxaborolan-2-yl)phenyl]carbamate (WO2011/25927, 2011, AI) (3.0 g, 8.0. mmol, 1.0 equiv), H₂O₂(19.3 g, 170 mmol, 16.4 mL, 30% purity, 21.1 equiv), NH₄Cl (4.3 g, 80 mmol, 10.0 equiv) in THF (30 mL), H₂O (30 mL) was degassed and purged with N₂ for 3 times, and then the mixture was stirred at 25° C. for 1 h under N₂ atmosphere. The mixture was added a solution of saturated Na₂SO₃ (100 ml) and stirred for 1 h. Then the mixture was extracted with EA (60 mL×2), the organic layer was washed with brine and dried over Na₂SO₄. The residue was purified by the column chromatography (SiO₂, Petroleum ether/Ethyl acetate=10/1 to 5/1) to give the tert-butyl N-(5-chloro-2-fluoro-3-hydroxy-phenyl)carbamate (2.0 g, 7.6 mmol, 95% yield) as a white solid.

Step 2. To a solution of tert-butyl N-(5-chloro-2-fluoro-3-hydroxy-phenyl)carbamate (1.0 g, 3.8 mmol, 1.0 equiv) in MeCN (10 mL) was added K₂CO₃ (528 mg, 3.8 mmol, 1.0 equiv) and added tert-butyl 2-(bromomethyl)prop-2-enoate (JACS, 2017, 139, 15308) (844 mg, 3.8 mmol, 1.0 equiv) and the mixture was stirred at 60° C. for 12 h. The mixture was diluted with water (20 mL), and the aqueous phase was extracted with ethyl acetate (20 mL×2). The combined organic phase were washed with brine (20 mL×2), dried over anhydrous sodium sulfate, filtered and concentrated under vacuum to give a residue. The residue was purified by the column chromatography (SiO₂, Petroleum ether/Ethyl acetate=10/1 to 5/1) to give tert-butyl 2-[[3-(tert-butoxycarbonylamino)-5-chloro-2-fluoro-phenoxy]methyl]prop-2-enoate (1.1 g, 2.7 mmol, 71% yield) as a yellow solid.

Step 3. To a solution of tert-butyl 2-[[3-(tert-butoxycarbonylamino)-5-chloro-2-fluoro-phenoxy]methyl]prop-2-enoate (900 mg, 2.2 mmol, 1.0 equiv) in EA (6 mL) was added HCl/dioxane (4 M, 559 μL, 1.0 equiv). The mixture was stirred at 25° C. for 1 h. The mixture was added H₂O (30 mL, the aqueous phase was extracted with EA (30 mL×3). The combined organic phase was washed with brine (30 mL×3), dried with anhydrous sodium sulfate, filtered and concentrated in vacuum. The residue was purified by column chromatography (SiO₂, Petroleum ether/Ethyl acetate=10/1 to 5/1) to give the tert-butyl 2-[(3-amino-5-chloro-2-fluoro-phenoxy)methyl]prop-2-enoate (240 mg, 795 umol, 35% yield) as a yellow solid.

Step 4. To a solution of TEA (167 mg, 1.7 mmol, 230 μL, 5.0 equiv) in THF (5 mL) and the mixture was cooled to −78° C. Then the mixture was added triphosgene (100 mg, 331 umol 1.0 equiv) and dropwised tert-butyl 2-[(3-amino-5-chloro-2-fluoro-phenoxy)methyl]prop-2-enoate (100 mg, 331 umol, 1.0 equiv). The mixture was stirred at −78° C. for 1 h. Then the mixture was added 3-[5-(aminomethyl)-1-oxo-isoindolin-2-yl]piperidine-2,6-dione (90 mg, 331 umol, 1.0 equiv). The mixture was stirred at 25° C. for 3 h. The mixture was diluted with water (20 mL), adjusted to pH=10 by NaHCO₃ (10 mL) and the aqueous phase was extracted with ethyl acetate (20 mL×2). The combined organic phase were washed with brine (20 mL×2), dried over anhydrous sodium sulfate, filtered and concentrated under vacuum to give a residue. The residue was purified by prep-HPLC (column: Phenomenex luna C18 150*25 mm*10 um; mobile phase: [water(FA)-ACN]; B %: 42%-72%, 15 min) to give the tert-butyl 2-[[5-chloro-3-[[2-(2,6-dioxo-3-piperidyl)-1-oxo-isoindolin-5-yl]methylcarbamoylamino]-2-fluoro-phenoxy]methyl]prop-2-enoate (85 mg, 141 umol, 42% yield) as a white solid. ¹H NMR: (400 MHz, DMSO-d6) δ=8.74 (s, 1H), 7.88-7.86 (m, 1H), 7.70-7. 68 (m, 1H), 7.51 (s, 1H), 7.44-7.38 (m, 2H), 6.91 (s, 1H), 6.88-6.86 (m, 1H), 6.21 (s, 1H), 5.92 (s, 1H), 5.11-5.07 (m, 1H), 4.46-4.28 (m, 4H), 2.91-2.90 (m, 1H), 2.60-2.56 (m, 1H), 2.39-2.36 (m, 1H), 2.03-1.92 (m, 1H), 1.43 (s, 9H)

Step 5. To a solution of tert-butyl 2-[[5-chloro-3-[[2-(2,6-dioxo-3-piperidyl)-1-oxo-isoindolin-5-yl]methylcarbamoylamino]-2-fluoro-phenoxy]methyl]prop-2-enoate (85 mg, 141 umol, 1.0 equiv) in DCM (3 mL) and TFA (1 mL). The mixture was stirred at 25° C. for 1 h. The mixture was concentrated under reduced pressure to give a residue. The residue was used for next step without further purification to give the 2-[[3-chloro-5-[[2-(2,6-dioxo-3-piperidyl)-6-fluoro-1-oxo-isoindolin-5-yl]methylcarbamoylamino]phenoxy]methyl]prop-2-enoic acid (60 mg, 91 umol, 91% yield, TFA) as a white solid.

### Compound AAN: 1-(5-chloro-2-hydroxy-4-(trifluoromethyl)phenyl)-3-((2-(2,6-dioxopiperidin-3-yl)-1-oxoisoindolin-5-yl)methyl)urea

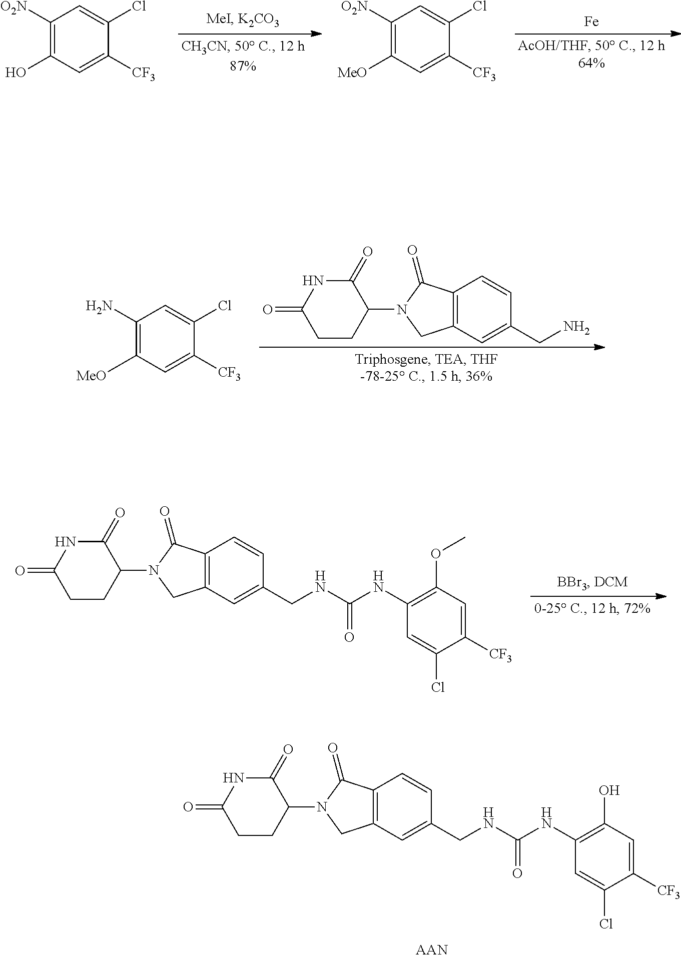

Step 1. To a solution of 4-chloro-2-nitro-5-(trifluoromethyl)phenol (US2017/298081, 2017, A1)(6.5 g, 26.9 mmol, 1 equiv) in MeCN (70 mL) was added K₂CO₃ (14.8 g, 107.6 mmol, 4.0 equiv) and Mel (11.4 g, 80.7 mmol, 5.0 mL, 3 equiv). The mixture was stirred at 50° C. for 12 h. TLC indicated Reactant 1 was consumed completely and one new spot formed. The residue was purified by column chromatography (SiO₂, Petroleum ether/Ethyl acetate=50/1 to 20/1) to give compound 1-chloro-4-methoxy-5-nitro-2-(trifluoromethyl)benzene (6 g, 87% yield) as a colorless oil. ¹H NMR (400 MHz, CDCl₃): δ 7.95 (s, 1H), 7.41 (s, 1H), 4.03 (s, 3H).

Step 2. To a solution of 1-chloro-4-methoxy-5-nitro-2-(trifluoromethyl)benzene (6 g, 23.5 mmol, 1.0 equiv) in THF (60 mL), CH₃COOH (30 mL) was added Fe (6.5 g, 117.4 mmol, 5.0 equiv). The mixture was stirred at 50° C. for 12 h. The residue was purified by prep-HPLC (column: Waters Xbridge C18 150*50 mm*10 um; mobile phase: [water (NH₄HCO₃)-ACN]; B %: 37%-67%, 10 min) to give compound 5-chloro-2-methoxy-4-(trifluoromethyl)aniline (3.4 g, 64% yield) as a colorless oil.

Step 3. To a solution of Triphosgene (3.2 g, 10.8 mmol, 0.8 equiv) in THF (200 mL) was added Et₃N (13.5 g, 132.9 mmol, 18.5 mL, 10 equiv) and 5-chloro-2-methoxy-4-(trifluoromethyl) aniline (3 g, 13.3 mmol, 1.0 equiv). The mixture was stirred at −78° C. for 0.5 h. Then 3-[5-(aminomethyl)-1-oxo-isoindolin-2-yl] piperidine-2,6-dione (J. Am. Chem. Soc., 2017, 139, 15308) (4.5 g, 14.6 mmol, 1.0 equiv, HCl) in THF (20 mL) was added. The mixture was stirred at −78° C. for 0.5 h. The mixture was stirred at 25° C. for 0.5 h. The mixture was added 100 ml H₂O and extracted with DCM/MeOH (10:1, 30 mL×3). The combined organic layers were filtered and concentrated under reduced pressure to give a residue. The residue was purified by prep-HPLC (column: Phenomenex luna C18 150*40 mm*15 um; mobile phase: [water (FA)-ACN]; B %: 40%-60%, 10 min) to give compound 1-[5-chloro-2-methoxy-4-(trifluoromethyl)phenyl]-3-[[2-(2,6-dioxo-3-piperidyl)-1-oxo-isoindolin-5-yl]methyl]urea (2.5 g, 36.0% yield) as a white solid.

Step 4. To a solution of 1-[5-chloro-2-methoxy-4-(trifluoromethyl) phenyl]-3-[[2-(2,6-dioxo-3-piperidyl)-1-oxo-isoindolin-5-yl] methyl] urea (114.5 mg, 218.4 umol, 1.0 equiv) in DCM (3 mL) was added BBr₃ (273.2 mg, 1.0 mmol, 105.1 uL, 5.0 equiv) at 0° C. The mixture was stirred at 25° C. for 12 h. The reaction mixture was concentrated under reduced pressure to remove solvent. The crude product was triturated with H₂O (10 mL) at 25° C. for 0.5 h. The reaction mixture was and concentrated under reduced pressure to give a residue. The crude product was triturated with THF (10 mL) at 25° C. for 0.5 h. After filtered, the filter cake was dried to give a compound 1-[5-chloro-2-hydroxy-4-(trifluoromethyl) phenyl]-3-[[2-(2,6-dioxo-3-piperidyl)-1-oxo-isoindolin-5-yl]methyl]urea (80 mg, 72% yield) as a white solid.

### Compound AAO: 1-((2-(2,6-dioxopiperidin-3-yl)-1-oxoisoindolin-5-yl)methyl)-3-(3-hydroxy-4-methoxyphenyl)urea

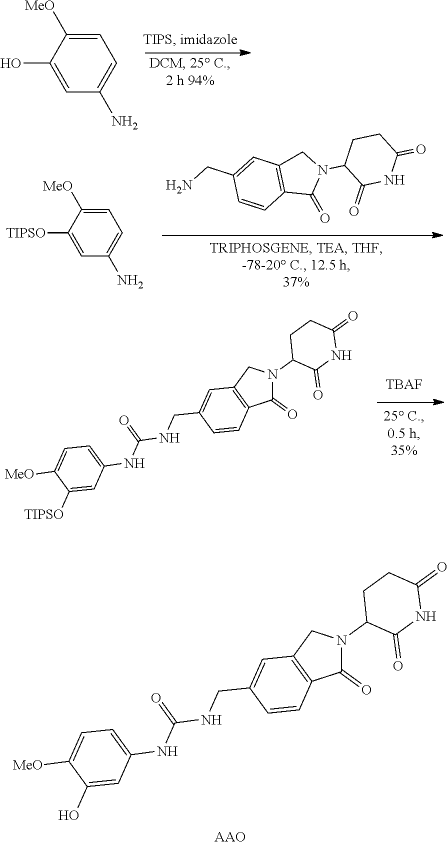

Step 1. To a solution of 5-amino-2-methoxy-phenol (500 mg, 3.59 mmol, 1.0 equiv) in DCM (10 mL) was added TIPSCl (1.39 g, 7.19 mmol, 2.0 equiv) and IMIDAZOLE (734 mg, 10.8 mmol, 3.0 equiv). The mixture was stirred at 25° C. for 12 h. The reaction mixture was concentrated to afford crude product. The residue was purified by silica chromatography (PE:EA=1:0-2:1) to afford 4-methoxy-3-triisopropylsilyloxy-aniline (1.0 g, 3.38 mmol, 94% yield) as a yellow oil.

Step 2. To a solution of TEA (685 mg, 6.77 mmol, 10.0 equiv) and triphosgene (277 mg, 933.5 umol, 1.4 equiv) in THF (8 mL) was added 4-methoxy-3-triisopropylsilyloxy-aniline (200 mg, 676.8 umol, 1.0 equiv) in THF (2 mL) at −78° C., and then it was stirred for 0.5 h. 3-[5-(aminomethyl)-1-oxo-isoindolin-2-yl]piperidine-2,6-dione (J. Am. Chem. Soc., 2017, 139, 15308)(210 mg, 677 umol, 1.0 equiv, HCl) was added to the mixture and then it was slowly warmed to 20° C. and stirred for 12 h. The residue was quenched with H₂O (50 mL) and extracted with EA (50 mL×3). The organic phase was concentrated to afford crude product. The residue was purified by prep-TLC (SiO₂, PE:EA=0:1) to afford 1-[[2-(2,6-dioxo-3-piperidyl)-1-oxo-isoindolin-5-yl]methyl]-3-(4-methoxy-3-triisopropylsilyloxy-phenyl)urea (150 mg, 252 umol, 37% yield) as a yellow oil

Step 3. To a solution of 1-[[2-(2,6-dioxo-3-piperidyl)-1-oxo-isoindolin-5-yl]methyl]-3-(4-methoxy-3-triisopropylsilyloxy-phenyl)urea (150 mg, 252 umol, 1.0 equiv) was added TBAF (1 M, 1 mL, 4.0 equiv). The mixture was stirred at 25° C. for 0.5 h. The reaction mixture was concentrated to afford crude product. The residue was purified by prep-HPLC (column: 3_Phenomenex Luna C18 75*30 mm*3 um; mobile phase: [water(TFA)-ACN]; B %: 14%-34%, 9 min) to afford 1-[[2-(2,6-dioxo-3-piperidyl)-1-oxo-isoindolin-5-yl]methyl]-3-(3-hydroxy-4-methoxy-phenyl)urea (40 mg, 89.4 umol, 35% yield, 98% purity), the product was a white solid. ¹H NMR (400 MHz, DMSO-d₆) δ 11.18-10.69 (m, 1H), 9.06-8.65 (m, 1H), 8.32 (s, 1H), 7.69 (d, J=7.8 Hz, 1H), 7.50 (s, 1H), 7.43 (d, J=7.8 Hz, 1H), 6.98 (d, J=2.1 Hz, 1H), 6.78-6.74 (m, 1H), 6.72-6.68 (m, 1H), 6.59-6.54 (m, 1H), 5.10 (m, 1H), 4.48-4.28 (m, 4H), 3.68 (s, 3H), 3.57-3.45 (m, 2H), 2.97-2.82 (m, 1H), 2.04-1.95 (m, 1H)

### Compound AAP: 1-(3-chloro-4-methylphenyl)-3-((2-(2,6-dioxopiperidin-3-yl)-4-hydroxy-1-oxoisoindolin-5-yl)methyl)urea

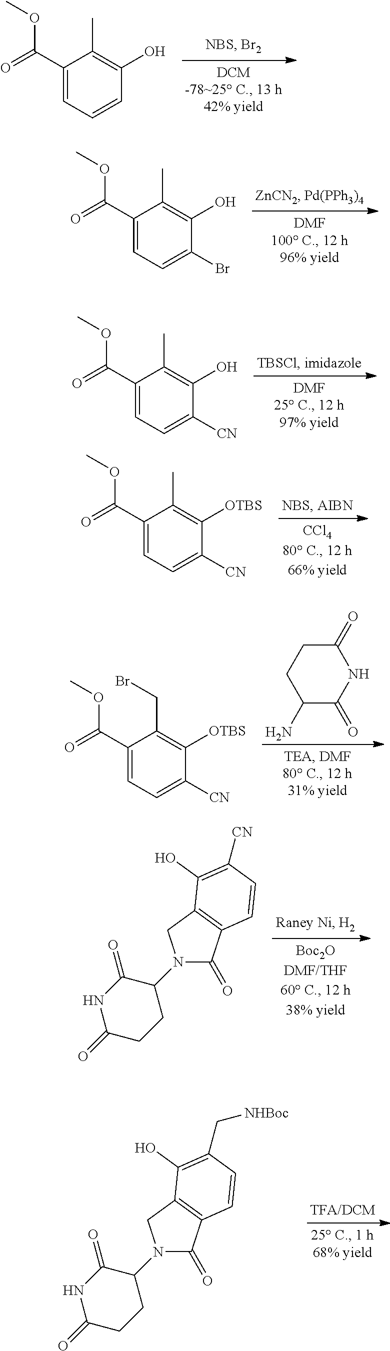

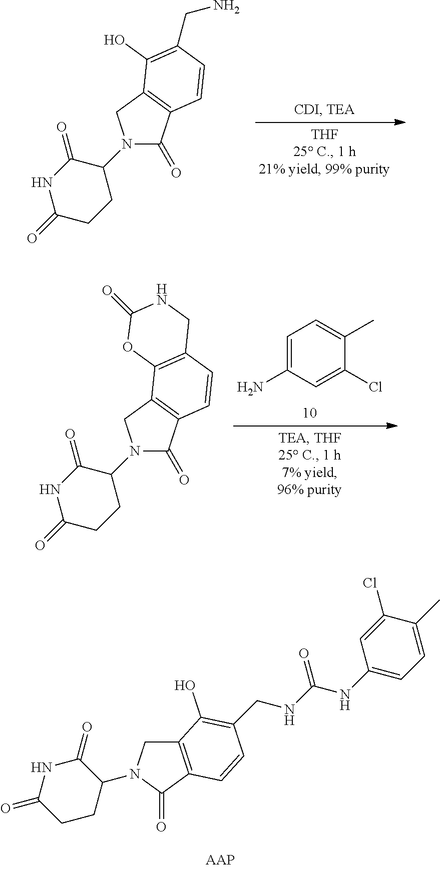

Step 1. To a solution of 2-methylpropan-2-amine (2.2 g, 30.1 mol, 3.2 mL, 1.0 equiv) in DCM (200 mL) was added dropwise Br₂ (4.8 g, 30.1 mmol, 1.6 mL, 1.0 equiv) in DCM (200 mL) at−78C. After addition, the mixture was stirred at this temperature for 1 h, and then methyl 3-hydroxy-2-methyl-benzoate (5.0 g, 30.1 mmol, 1.0 equiv) in DCM (200 mL) was added dropwise at −78° C. The resulting mixture was stirred at 25° C. for 12 h. The reaction mixture filtered and concentrated to give the residue. The residue was purified by flash silica gel chromatography (ISCO®; 5 g SepaFlash® Silica Flash Column, Eluent of 0-7% Ethyl acetate/Petroleum ethergradient @ 60 mL/min) and purified by prep-HPLC (column: YMC Triart C18 250*50 mm*7 um; mobile phase: [water(FA)-ACN]; B %: 24%-54%, 20 min) to give the methyl 4-bromo-3-hydroxy-2-methyl-benzoate (3.3 g, 13.5 mmol, 42% yield) as a yellow solid. ¹H NMR: (400 MHz, CDCl₃) δ=7.39-7.28 (m, 2H), 3.90 (d, J=1.2 Hz, 3H), 2.53 (s, 3H).

Step 2. A mixture of methyl 4-bromo-3-hydroxy-2-methyl-benzoate (5 g, 20.4 mmol, 1.0 equiv), Zn(CN)₂ (5.4 g, 46 mmol, 2.9 mL, 2.3 equiv), Pd(PPh₃)₄(2.4 g, 2 mmol, 0.1 equiv) in DMF (50 mL) was degassed and purged with N₂ for 3 times, and then the mixture was stirred at 100° C. for 12 h under N₂ atmosphere. The reaction mixture filtered and concentrated to get the residue together. The residue was diluted with H₂O (10 mL) and extracted with DCM (10 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3/1 to 1/1) to give the methyl 4-cyano-3-hydroxy-2-methyl-benzoate (7.5 g, 39 mmol, 96% yield) as a yellow solid.

Step 3. To a stirred solution of methyl 4-cyano-3-hydroxy-2-methyl-benzoate (4.5 g, 23.5 mmol, 1.0 equiv) in DMF (45 mL) was added imidazole (1.9 g, 28.3 mmol, 1.2 equiv) under N₂ atmosphere. Then the mixture was cooled to 0° C., and then the mixture was added dropwise tert-butyl-chloro-dimethyl-silane (4.3 g, 28.3 mmol, 3.5 mL, 1.2 equiv). The mixture was stirred under N₂ at 25° C. for 12 h. The reaction mixture filtered and concentrated to get the residue together. The residue was diluted with H₂O (10 mL) and extracted with DCM (10 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=1/20 to 10/1) to the methyl 3-[tert-butyl (dimethyl)silyl]oxy-4-cyano-2-methyl-benzoate (14 g, 45.8 mmol, 97% yield) as a light yellow oil. ¹H NMR: (400 MHz, CDCl₃) δ=7.42-7.27 (m, 2H), 3.82 (s, 3H), 2.31 (s, 3H), 0.98 (s, 9H), 0.20 (s, 6H).

Step 4. A mixture of methyl 3-[tert-butyl(dimethyl)silyl]oxy-4-cyano-2-methyl-benzoate (3.0 g, 10.0 mmol, 1.0 equiv), NBS (3.5 g, 19.6 mmol, 2.0 equiv), AIBN (1.61 g, 9.8 mmol, 1.0 equiv) in CCl₄ (30 mL) was degassed and purged with N₂ for 3 times, and then the mixture was stirred at 80° C. for 12 h under N₂ atmosphere. The reaction mixture filtered and concentrated to get the residue together. The residue was diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by flash silica gel chromatography (ISCO®; 4.3 g SepaFlash® Silica Flash Column, Eluent of 0-9% Ethyl acetate/Petroleum ethergradient @ 60 mL/min) to give the methyl 2-(bromomethyl)-3-[tert-butyl (dimethyl) silyl]oxy-4-cyano-benzoate (2.5 g, 6.5 mmol, 66% yield) as a yellow solid. ¹H NMR: (400 MHz, CDCl₃) δ=7.55 (d, J=0.8 Hz, 2H), 4.94 (s, 2H), 3.98 (s, 3H), 1.13-1.09 (m, 9H), 0.44-0.33 (m, 6H).

Step 5. A mixture of methyl 2-(bromomethyl)-3-[tert-butyl(dimethyl)silyl]oxy-4-cyano-benzoate (2.5 g, 6.5 mmol, 1.0 equiv), 3-aminopiperidine-2,6-dione (1.1 g, 6.5 mmol, 1.0 equiv, HCl), TEA (2.0 g, 19.5 mmol, 2.7 mL, 3.0 equiv) in DMF (20 mL) was degassed and purged with N₂ for 3 times, and then the mixture was stirred at 80° C. for 12 h under N₂ atmosphere. The mixture was concentrated to get the residue. The residue was purified by prep-HPLC (column: YMC Triart C18 250*50 mm*7 um; mobile phase: [water(FA)-ACN]; B %: 5%-32%, 22 min) to give the 2-(2,6-dioxo-3-piperidyl)-4-hydroxy-1-oxo-isoindoline-5-carbonitrile (580 mg, 2 mmol, 31% yield) as a brown solid. ¹H NMR: (400 MHz, DMSO-d6) δ=11.00-10.55 (m, 1H), 10.18 (s, 1H), 6.92 (d, J=8.0 Hz, 1H), 6.44 (d, J=7.8 Hz, 1H), 4.32-4.23 (m, 1H), 3.65-3.43 (m, 2H), 2.11-2.00 (m, 1H), 1.76 (d, J=17.2 Hz, 1H), 1.57-1.44 (m, 1H), 1.23-1.14 (m, 1H).

Step 6. A mixture of 2-(2,6-dioxo-3-piperidyl)-4-hydroxy-1-oxo-isoindoline-5-carbonitrile (580 mg, 2.0 mmol, 1.0 equiv), Boc₂O (1.3 g, 6.1 mmol, 1.4 mL, 3.0 equiv), Raney-Ni (174 mg, 2.0 mmol, 1.0 equiv) in DMF (25 mL), THF (50 mL) was degassed and purged with N₂ for 3 times, and then the mixture was stirred at 60° C. for 12 h under H₂ atmosphere. The mixture was cooled to 0° C., then the mixture was filtered. The filtrate was concentrated to get the residue. The residue was purified by prep-HPLC (column: Phenomenex luna C18 250*50 mm*10 um; mobile phase: [water(TFA)-ACN]; B %: 31%-61%, 20 min) to give the tert-butyl N-[[2-(2,6-dioxo-3-piperidyl)-4-hydroxy-1-oxo-isoindolin-5-yl]methyl]carbamate (300 mg, 770 umol, 38% yield) as a light yellow solid. ¹H NMR: (400 MHz, CDCl₃) δ=7.86 (s, 1H), 7.63 (d, J=7.6 Hz, 1H), 7.42 (d, J=7.2 Hz, 1H), 5.12-5.02 (m, 1H), 4.81 (s, 1H), 4.38-4.07 (m, 5H), 2.83-2.63 (m, 2H), 2.29-2.04 (m, 2H), 1.31 (s, 9H).

Step 7. A mixture of tert-butyl N-[[2-(2,6-dioxo-3-piperidyl)-4-hydroxy-1-oxo-isoindolin-5-yl]methyl]carbamate (100 mg, 256.80 umol, 1.0 equiv) in TFA (1 mL), DCM (3 mL) was degassed and purged with N₂ for 3 times, and then the mixture was stirred at 25° C. for 1 hr under N₂ atmosphere. The mixture was concentrated to get the residue. The residue was used for next step without further purification to give the 3-[5-(aminomethyl)-4-hydroxy-1-oxo-isoindolin-2-yl] piperidine-2,6-dione (70 mg, 174 umol, 68% yield, TFA) was obtained as a yellow oil.

Step 8. To a solution of 3-[5-(aminomethyl)-4-hydroxy-1-oxo-isoindolin-2-yl]piperidine-2,6-dione (37 mg, 128 umol, 1.0 equiv) in THF (1 mL) was added CDI (42 mg, 256 umol, 2.0 equiv) and TEA (39 mg, 384 umol, 54 μL, 3.0 equiv). The mixture was stirred at 25° C. for 1 h. The mixture was concentrated to get the residue. The residue was purified by prep-HPLC (column: Phenomenex Luna C18 150*25 mm*10 um; mobile phase: [water(FA)-ACN]; B %: 1%-30%, min) to give the 8-(2,6-dioxo-3-piperidyl)-4,9-dihydro-3H-pyrrolo[3,4-h][1,3]benzoxazine-2,7-dione (9 mg, 27 umol, 21% yield, 99% purity) as a white solid. ¹H NMR: (400 MHz, DMSO-d6) δ=10.98 (d, J=0.8 Hz, 1H), 8.46 (s, 1H), 8.14 (s, 1H), 7.50 (d, J=7.6 Hz, 1H), 7.40 (d, J=7.6 Hz, 1H), 5.16-4.98 (m, 1H), 4.56-4.45 (m, 3H), 4.34-4.27 (m, 1H), 2.97-2.85 (m, 1H), 2.63-2.60 (m, 1H), 2.47-2.40 (m, 1H), 2.04-1.95 (m, 1H).

Step 9. To a solution of 3-[5-(aminomethyl)-4-hydroxy-1-oxo-isoindolin-2-yl]piperidine-2,6-dione (37 mg, 128 umol, 1.0 equiv) in THF (1 mL) was added TEA (65 mg, 640 umol, 89 μL, 5.0 equiv) and 2-chloro-4-isocyanato-1-methyl-benzene (21 mg, 128 umol, 1.0 equiv) at 0° C. The mixture was stirred at 25° C. for 1 h. The mixture was concentrated to get the residue. The residue was purified by prep-HPLC (column: Phenomenex luna C18 150*25 mm*10 um; mobile phase: [water(FA)-ACN]; B %: 25%-55%, 15 min) to give the 1-(3-chloro-4-methyl-phenyl)-3-[[2-(2,6-dioxo-3-piperidyl)-4-hydroxy-1-oxo-isoindolin-5-yl]methyl]urea (4 mg, 9 umol, 7% yield, 96% purity) as a yellow solid. ¹H NMR: (400 MHz, DMSO-d6) δ=10.99 (s, 1H), 10.29-9.90 (m, 1H), 8.92 (s, 1H), 7.63 (d, J=1.6 Hz, 1H), 7.35 (d, J=7.6 Hz, 1H), 7.26-7.07 (m, 3H), 6.87 (s, 1H), 5.19-5.06 (m, 1H), 4.43-4.19 (m, 4H), 2.98-2.86 (m, 1H), 2.69-2.57 (m, 1H), 2.43-2.35 (m, 1H), 2.24 (s, 3H), 2.06-1.95 (m, 1H).

### Compound AAQ: 1-(2-chloro-3-hydroxyphenyl)-3-((2-(2,6-dioxopiperidin-3-yl)-1-oxoisoindolin-5-yl)methyl)urea

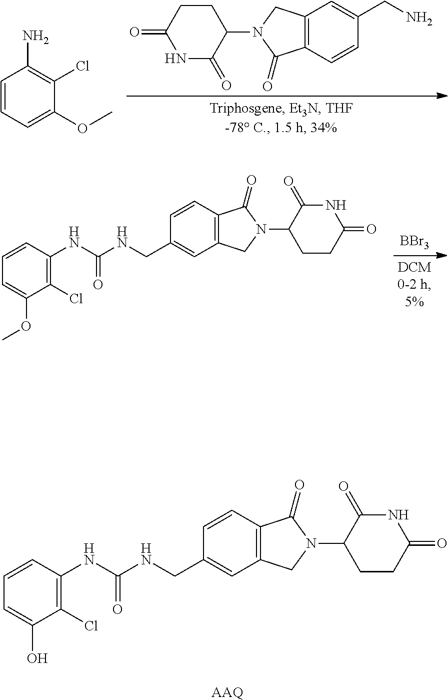

Step 1. To a solution of triphosgene (1.0 g, 3.4 mmol, 0.5 equiv) in THF (200 mL) was added Et₃N (4.5 g, 44.4 mmol, 6.2 mL, 10.0 equiv) and 2-chloro-3-methoxy-aniline (0.7 g, 4.4 mmol, 1.0 equiv) in THF (10 mL). The mixture was stirred at −78° C. for 0.5 h. Then 3-[5-(aminomethyl)-1-oxo-isoindolin-2-yl] piperidine-2,6-dione (J. Am. Chem. Soc., 2017, 139, 15308)(1.2 g, 4.44 mmol, 1 equiv) was added. The mixture was stirred at −78° C. for 0.5 h, and stirred at 25° C. for 0.5 h. The mixture was added 50 mL H₂O and extracted with DCM/MeOH (10:1, 50 mL×3). The combined organic layers were filtered and concentrated under reduced pressure to give a residue. The residue was purified by prep-TLC (SiO₂, DCM: MeOH=10:1) to give compound 1-(2-chloro-3-methoxy-phenyl)-3-[[2-(2,6-dioxo-3-piperidyl)-1-oxo-isoindolin-5-yl] methyl] urea (0.7 g, 1.5 mmol, 34% yield) as a colorless oil.

Step 2. To a solution of 1-(2-chloro-3-methoxy-phenyl)-3-[[2-(2,6-dioxo-3-piperidyl)-1-oxo-isoindolin-5-yl] methyl] urea (200 mg, 437.8 umol, 1 equiv) in DCM (3 mL) was added BBr₃ (548.3 mg, 2.2 mmol, 210.9 uL, 5 equiv). The mixture was stirred at 0° C. for 2 h. The reaction mixture was added 0.1 ml H₂O at 0° C. and concentrated under reduced pressure to remove solvent. The residue was purified by prep-HPLC (column: Phenomenex C18 75*30 mm*3 um; mobile phase: [water (FA)-ACN]; B %: 10%-40%, 7 min) to give compound 1-(2-chloro-3-hydroxy-phenyl)-3-[[2-(2,6-dioxo-3-piperidyl)-1-oxo-isoindolin-5-yl]methyl]urea (10 mg, 5.07% yield) as a white solid. ¹H NMR (400 MHz, DMSO-d₆): δ 10.83-11.45 (m, 1H), 9.55-10.41 (m, 1H), 8.05 (s, 1H), 7.71 (d, J=7.82 Hz, 1H), 7.58-7.67 (m, 2H), 7.53 (s, 1H), 7.45 (d, J=7.95 Hz, 1H), 6.98-7.02 (m, 1H), 6.57 (m, 1H), 5.11 (m, 1H), 4.29-4.45 (m, 4H), 2.87-3.01 (m, 1H), 2.59-2.67 (m, 1H), 2.31-2.37 (m, 1H), 1.94-2.05 (m, 1H).

### Compound AAR: 1-(1-chloro-6-hydroxynaphthalen-2-yl)-3-((2-(2,6-dioxopiperidin-3-yl)-1-oxoisoindolin-5-yl)methyl)urea

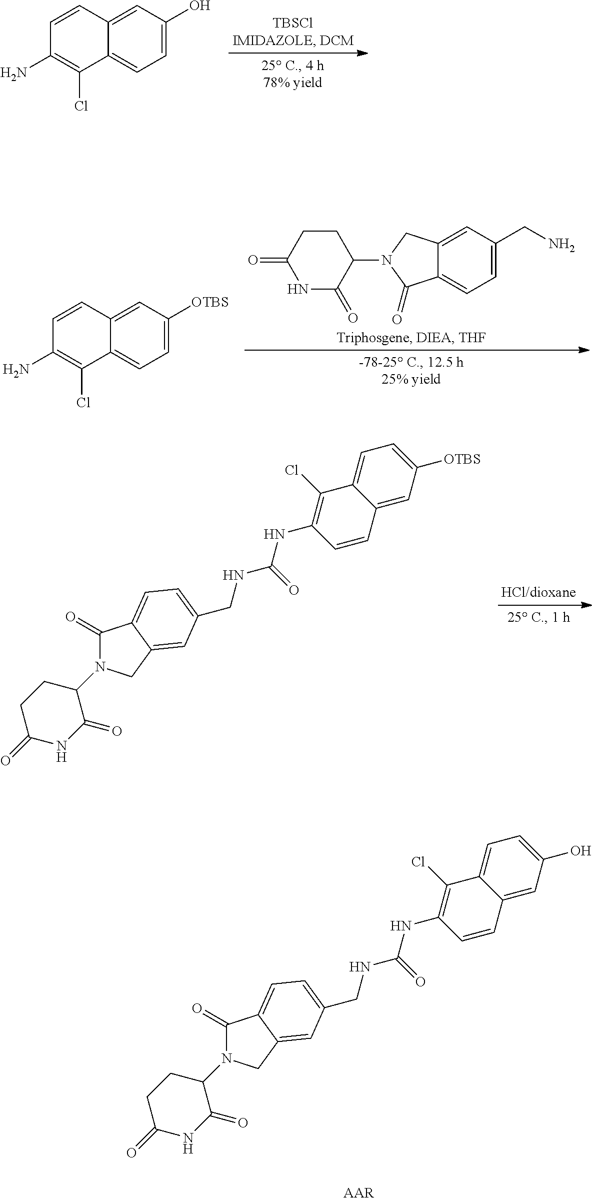 [SMILES: Nc1ccc2cc(O)ccc2c1Cl ; CC(C)(C)[Si](C)(C)Oc1ccc2c(Cl)c(N)ccc2c1 ; NCc1ccc2c(c1)CN(C1CCC(=O)NC1=O)C2=O ; CC(C)(C)[Si](C)(C)Oc1ccc2c(Cl)c(NC(=O)NCc3ccc4c(c3)CN(C3CCC(=O)NC3=O)C4=O)ccc2c1 ; O=C1CCC(N2Cc3cc(CNC(=O)Nc4ccc5cc(O)ccc5c4Cl)ccc3C2=O)C(=O)N1 ; "AAR"]

Step 1. To a solution of 6-amino-5-chloro-naphthalen-2-ol (described in WO 2011/25927) (120 mg, 619 umol, 1.0 equiv) in DCM (2 mL) was added TBSCl (186 mg, 1 mmol, 151 uL, 2.0 equiv) and imidazole (126 mg, 1 mmol, 3.0 equiv). The mixture was stirred at 25° C. for 4 h. The mixture was added 30 mL water, the aqueous phase was extracted with methylene dichloride (30 mL×3). The combined organic phase was washed with brine (30 mL×3), dried with anhydrous sodium sulfate, filtered and concentrated in vacuum. The residue was purified by column chromatography (SiO₂, Petroleum ether/Ethyl acetate=20/1 to 10/1) to give the 6-[tert-butyl(dimethyl)silyl]oxy-1-chloro-naphthalen-2-amine (150 mg, 487 umol, 78% yield) as a yellow oil. ¹H NMR: (400 MHz, DMSO-d₆) δ=7.76 (d, J=9.2 Hz, 1H), 7.52 (d, J=8.8 Hz, 1H), 7.23-7.02 (m, 3H), 5.51 (s, 2H), 0.97 (s, 9H), 0.20 (s, 6H)

Step 2. To a solution of triphosgene (140 mg, 471 umol, 2.0 equiv) in THF (4 mL) and the mixture was cooled to −78° C. Then the mixture was added TEA (230 mg, 2 mmol, 316 μL, 10.0 equiv) and 6-[tert-butyl(dimethyl)silyl]oxy-1-chloro-naphthalen-2-amine (70 mg, 227 umol, 1.0 equiv) was dropwised to the mixture. The mixture was stirred at −78° C. for 0.5 h. Then the mixture was added 3-[5-(aminomethyl)-1-oxo-isoindolin-2-yl]piperidine-2,6-dione (62 mg, 227 umol, 1.0 equiv). The mixture was stirred at 25° C. for 12 h. The resultant mixture was diluted with water (20 mL), adjust the pH of the solution to 10 with NaHCO₃ (10 mL) and the aqueous phase was extracted with ethyl acetate (20 mL×2). The combined organic phase was washed with brine (20 mL×2), dried over anhydrous sodium sulfate, filtered and concentrated under vacuum to give a residue. The residue was purified by prep-HPLC (column: Phenomenex luna C18 150*25 mm*10 um; mobile phase: [water(FA)-ACN]; B %: 62%-92%, 15 min) to give the 1-[6-[tert-butyl(dimethyl)silyl]oxy-1-chloro-2-naphthyl]-3-[[2-(2,6-dioxo-3-piperidyl)-1-oxo-isoindolin-5-yl]methyl]urea (35 mg, 57 umol, 25% yield) as a white solid. ¹H NMR: (400 MHz, CDCl₃) δ=8.18 (d, J=9.2 Hz, 1H), 8.10-7.98 (m, 2H), 7.73-7.57 (m, 2H), 7.47 (s, 1H), 7.36 (d, J=8.0 Hz, 1H), 7.22 (s, 1H), 7.18-7.14 (m, 2H), 5.28-5.07 (m, 1H), 4.65-4.49 (m, 2H), 4.48-4.36 (m, 1H), 4.33-4.21 (m, 1H), 2.98-2.70 (m, 2H), 2.40-2.16 (m, 2H), 1.02 (s, 9H), 0.25 (s, 6H)

Step 3. To a solution of 1-[6-[tert-butyl(dimethyl)silyl]oxy-1-chloro-2-naphthyl]-3-[[2-(2,6-dioxo-3-piperidyl)-1-oxo-isoindolin-5-yl]methyl]urea (35 mg, 57 umol, 1.0 equiv) inand HC/dioxane (3 mL). The mixture was stirred at 25° C. for 1 h. The reaction mixture was concentrated under reduced pressure to give a residue. The residue was purified by prep-HPLC (column: Phenomenex luna C18 150*25 mm*10 um; mobile phase: [water(FA)-ACN]; B %: 13%-43%, 15 min) to give the 1-(1-chloro-6-hydroxy-2-naphthyl)-3-[[2-(2,6-dioxo-3-piperidyl)-1-oxo-isoindolin-5-yl]methyl]urea (8 mg, 15 umol, 27% yield, 96% purity) as a white solid. ¹H NMR: (400 MHz, MeOD) δ=8.11-7.90 (m, 2H), 7.79 (d, J=8.0 Hz, 1H), 7.62-7.47 (m, 3H), 7.26-7.07 (m, 2H), 5.23-5.04 (m, 1H), 4.58-4.43 (m, 4H), 3.02-2.74 (m, 2H), 2.56-2.38 (m, 1H), 2.25-2.10 (m, 1H)

### Compound AAS: 1-(5-chloro-2-fluoro-3-hydroxyphenyl)-3-((2-(2,6-dioxopiperidin-3-yl)-6-fluoro-1-oxoisoindolin-5-yl)methyl)urea

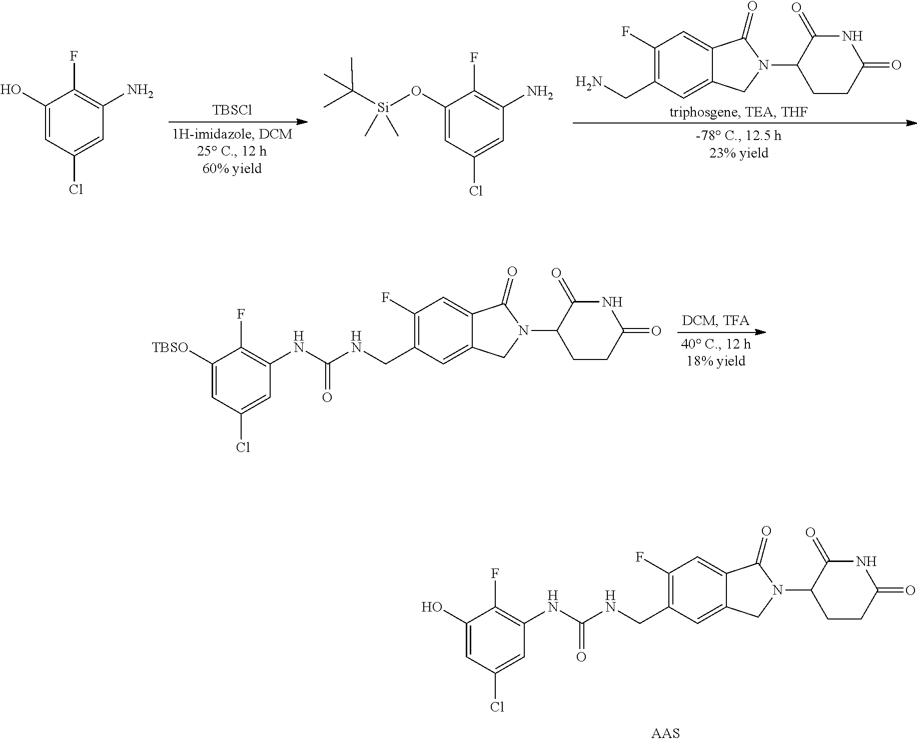

Step 1. To a mixture of DCM (10 mL), 3-amino-5-chloro-2-fluorophenol (described in US2020/407344) (320 mg, 200 umol, 1.0 equiv), imidazole (163 mg, 240 umol, 1.2 equiv) was stirred at 0° C. under N₂ protection, then the TBSCI (360 mg, 240 umol, 1.2 equiv) was added to the mixture. Then the mixture was stirred for 12 h at 25° C. under N₂ protection. The resultant mixture was diluted with water (20 mL), adjust the PH of the solution to 10 with NaHCO₃ (10 mL) and the aqueous phase was extracted with ethyl acetate (20 mL×2). The combined organic phase was washed with brine (20 mL×2), dried over anhydrous sodium sulfate, filtered and concentrated under vacuum to give a residue. The residue was purified by column chromatography (SiO₂, Petroleum ether/Ethyl acetate=50/1 to 3/1). to give the 3-((tert-butyldimethylsilyl)oxy)-5-chloro-2-fluoroaniline (330 mg, 120 umol, 60% yield) as a colorless oil.

Step 2. To a solution of triphosgene (80 mg, 269 umol, 0.5 equiv) in THF (5 mL) and the mixture was cooled to −78° C. Then the mixture was added TEA (366 mg, 3.0 mmol, 504 μL, 10.0 equiv) and dropwised3-[tert-butyl(dimethyl)silyl]oxy-5-chloro-2-fluoro-aniline (100 mg, 362 umol, 1.0 equiv). The mixture was stirred at −78° C. for 0.5 h. Then the mixture was added 3-[5-(aminomethyl)-6-fluoro-1-oxo-isoindolin-2-yl]piperidine-2,6-dione (105 mg, 362 umol, 1.0 equiv). The mixture was stirred at −78° C. for 12 h. The resultant mixture was diluted with water (20 mL), adjust the pH of the solution to 10 with NaHCO₃ (10 mL) and the aqueous phase was extracted with ethyl acetate (20 mL×2). The combined organic phase was washed with brine (20 mL×2), dried over anhydrous sodium sulfate, filtered and concentrated under vacuum to give a residue. The residue was purified by prep-HPLC (column: Phenomenex luna C18 150*25 mm*10 um; mobile phase: [water(FA)-ACN]; B %: 58%-88%, 15 min) to give the 1-[3-[tert-butyl(dimethyl)silyl]oxy-5-chloro-2-fluoro-phenyl]-3-[[2-(2,6-dioxo-3-piperidyl)-6-fluoro-1-oxo-isoindolin-5-yl]methyl]urea (50 mg, 84 umol, 23% yield) as a white solid. ¹H NMR: (400 MHz, CDCl₃) δ=7.91 (s, 1H), 7.84-7.68 (m, 1H), 7.57 (d, J=5.6 Hz, 1H), 7.41 (d, J=9.2 Hz, 1H), 6.78 (s, 1H), 6.64-6.52 (m, 1H), 5.61-5.48 (m, 1H), 5.28-5.04 (m, 1H), 4.66-4.50 (m, 2H), 4.47-4.26 (m, 2H), 3.02-2.76 (m, 2H), 2.49-2.14 (m, 2H), 0.99 (s, 9H), 0.19 (s, 6H)

Step 3. To a solution of 1-[3-[tert-butyl(dimethyl)silyl]oxy-5-chloro-2-fluoro-phenyl]-3-[[2-(2,6-dioxo-3-piperidyl)-6-fluoro-1-oxo-isoindolin-5-yl]methyl]urea (40 mg, 67 umol, 1.0 equiv) in DCM (2 mL) and TFA (1 mL). The mixture was stirred at 40° C. for 12 h. The reaction mixture was concentrated under reduced pressure to give a residue. The residue was purified by prep-HPLC (column: Waters Xbridge BEH C18 100*30 mm*10 um; mobile phase: [water(TFA)-ACN]; B %: 15%-45%, 10 min) to give the 1-(5-chloro-2-fluoro-3-hydroxy-phenyl)-3-[[2-(2,6-dioxo-3-piperidyl)-6-fluoro-1-oxo-isoindolin-5-yl]methyl]urea (6 mg, 12 umol, 18% yield, 99% purity) as a white solid. ¹H NMR: (400 MHz, DMSO-d₆) δ=10.99 (s, 1H), 10.28 (s, 1H), 8.61 (s, 1H), 7.72-7.64 (m, 1H), 7.59 (d, J=6.4 Hz, 1H), 7.53 (d, J=9.0 Hz, 1H), 7.31-7.21 (m, 1H), 6.56-6.51 (m, 1H), 5.18-5.06 (m, 1H), 4.49-4.44 (m, 1H), 4.43 (s, 2H), 4.34 (s, 1H), 2.93-2.88 (m, 1H), 2.59 (d, J=17.6 Hz, 1H), 2.36 (s, 1H), 2.00 (d, J=5.2 Hz, 1H)

### Compound AAT: 1-(4-chloro-3-hydroxy-5-methoxyphenyl)-3-((2-(2,6-dioxopiperidin-3-yl)-1-oxoisoindolin-5-yl)methyl)urea

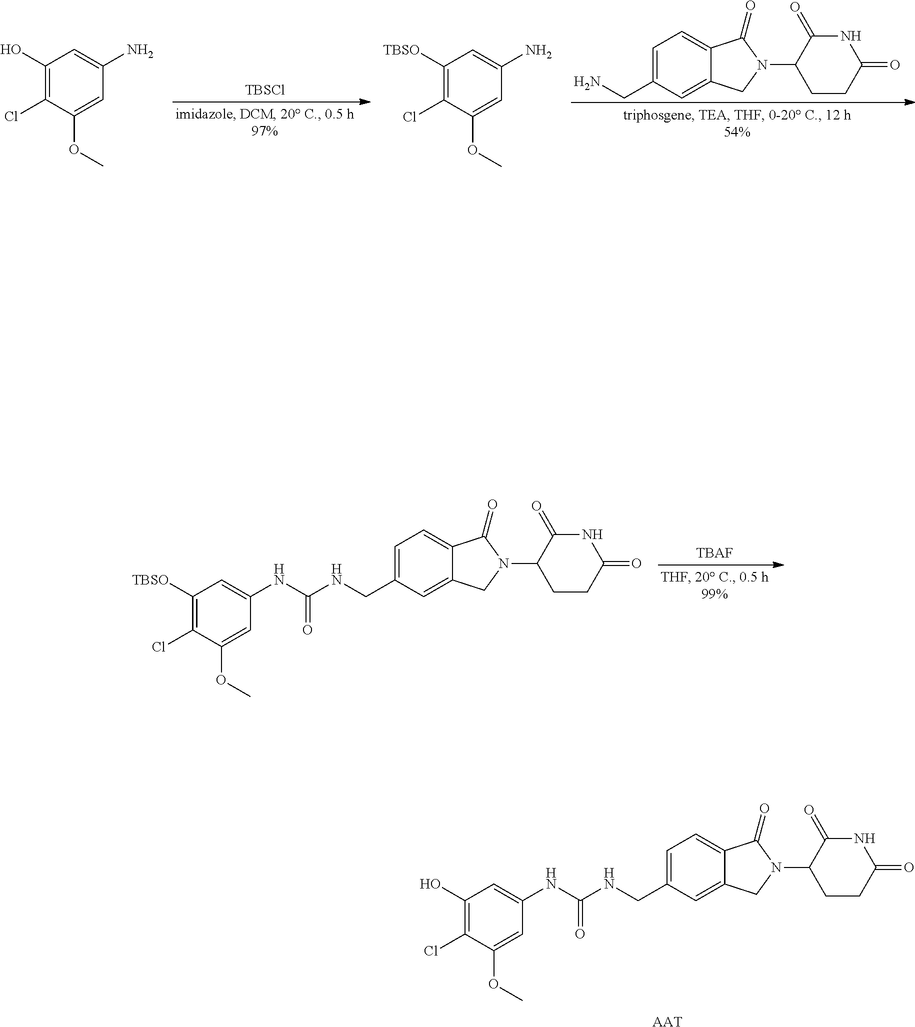

Step 1. To a solution of 5-amino-2-chloro-3-methoxy-phenol (130 mg, 748 umol, 1.0 equiv) in DCM (10 mL) was added imidazole (153 mg, 2.25 mmol, 3.0 equiv) and tert-butyl-chloro-dimethyl-silane (225 mg, 1.50 mmol, 183 μL, 2.0 equiv). The mixture was stirred at 20° C. for 0.5 h. The solution was purified by prep-TLC (PE/EA=2/1) to give 3-[tert-butyl(dimethyl) silyl]oxy-4-chloro-5-methoxy-aniline (210 mg, 97% yield).

Step 2. To a solution of bis(trichloromethyl) carbonate (371 mg, 1.25 mmol, 2.0 equiv) in THF (40 mL) was added 3-[tertbutyl(dimethyl)silyl]oxy-4-chloro-5-methoxy-aniline (180 mg, 625 umol, 1.0 equiv) and TEA (316 mg, 3.13 mmol, 435 μL, 5.0 equiv) at 0° C., and it was stirred for 0.5 h. Then 3-[5-(aminomethyl)-1-oxo-isoindolin-2-yl]piperidine-2,6-dione (213 mg, 688 umol, 1.1 equiv, HCl salt) was added to the mixture and it was stirred at 20° C. for 11.5 h. The solution was purified by prep-TLC (DCM/MeOH=10/1) to give 1-[3-[tert-butyl(dimethyl)silyl]oxy-4-chloro-5-methoxy-phenyl]-3-[[2-(2,6-dioxo-3-piperidyl)-1-oxo-isoindolin-5-yl]methyl]urea (200 mg, 54% yield) as a yellow gum.

Step 3. To a solution of 1-[3-[tert-butyl(dimethyl)silyl]oxy-4-chloro-5-methoxy-phenyl]-3-[[2-(2,6-dioxo-3-piperidyl)-1-oxo-isoindolin-5-yl]methyl]urea (140 mg, 238 umol, 1.0 equiv) in THF (5 mL) was added TBAF (1 M, 715 μL, 3.0 equiv). The mixture was stirred at 20° C. for 0.5 h. The reaction mixture was concentrated and was triturated with H2O/EA (1/1, 5 mL) at 20° C. for 30 min to give 1-(4-chloro-3-hydroxy-5-methoxy-phenyl)-3-[[2-(2,6-dioxo-3-piperidyl)-1-oxo-isoindolin-5-yl]methyl]urea (112 mg, 99% yield) as a white solid. ¹H NMR (400 MHz, DMSO-d₆): δ 11.08-10.91 (m, 1H), 10.00-9.86 (m, 1H), 8.69 (s, 1H), 7.76-7.67 (m, 1H), 7.57-7.40 (m, 2H), 6.87-6.62 (m, 3H), 5.18-5.05 (m, 1H), 4.48-4.29 (m, 4H), 3.74 (s, 3H), 2.94-2.87 (m, 2H), 2.47-2.36 (m, 2H)

### Compound AAU: 1-(4-chloro-2-hydroxyphenyl)-3-((2-(2,6-dioxopiperidin-3-yl)-1-oxoisoindolin-5-yl)methyl)urea

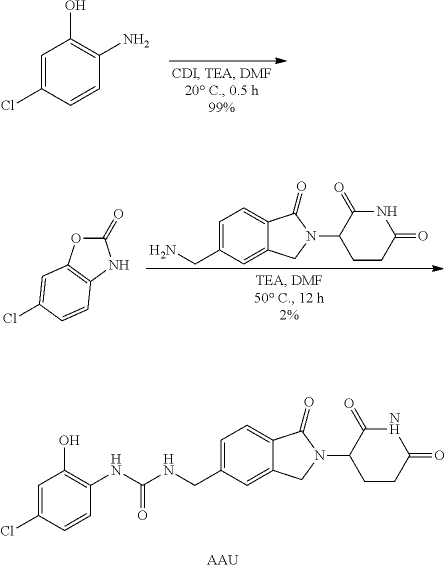 [SMILES: Nc1ccc(Cl)cc1O ; O=c1[nH]c2ccc(Cl)cc2o1 ; NCc1ccc2c(c1)CN(C1CCC(=O)NC1=O)C2=O ; O=C1CCC(N2Cc3cc(CNC(=O)Nc4ccc(Cl)cc4O)ccc3C2=O)C(=O)N1 ; "AAU"]

Step 1. To a solution of 2-amino-5-chloro-phenol (100 mg, 696 umol, 1.0 equiv) in DMF (1 mL) was added TEA (211 mg, 2.09 mmol, 290 μL, 3.0 equiv) and CDI (135 mg, 835 umol, 1.2 equiv). The mixture was stirred at 25° C. for 0.5 h. The crude product was used into the next step without further purification. Compound 6-chloro-3H-1,3-benzoxazol-2-one (118 mg, 695 umol, 99% yield) was obtained as a yellow oil.

Step 2. To a solution of 6-chloro-3H-1,3-benzoxazol-2-one (118 mg, 695 umol, 1.0 equiv) in DMF (1 mL) was added TEA (140 mg, 1.39 mmol, 193 μL, 2.0 equiv) and 3-[5-(aminomethyl)-1-oxo-isoindolin-2-yl]piperidine-2,6-dione (171 mg, 626 umol, 0.9 equiv). The mixture was stirred at 50° C. for 12 h. The mixture was concentrated to give a residue. The residue was purified by prep-HPLC (column: Phenomenex luna C18 150*25 mm*10 um; mobile phase: [water(FA)-ACN]; B %: 18%-48%, 10 min) to give crude product. The crude product was further purified by re-crystallization (30 mL, DCM/MeOH=10/1). Compound 1-(4-chloro-2-hydroxy-phenyl)-3-[[2-(2,6-dioxo-3-piperidyl)-1-oxo-isoindolin-5-yl]methyl]urea (8.22 mg, 17.3 umol, 2% yield, 93% purity) was obtained as a white solid. ¹H NMR (400 MHz, DMSO-d₆) δ=10.98 (s, 1H), 10.44 (s, 1H), 8.09 (s, 1H), 7.98 (d, J=8.8 Hz, 1H), 7.70 (d, J=8.0 Hz, 1H), 7.51 (s, 1H), 7.48-7.40 (m, 2H), 6.80 (d, J=2.0 Hz, 1H), 6.76-6.71 (m, 1H), 5.10 (m, 1H), 4.48-4.37 (m, 3H), 4.36-4.27 (m, 1H), 3.19-3.15 (m, 1H), 2.98-2.84 (m, 1H), 2.64-2.59 (m, 1H), 2.38 (m, 1H), 2.03-1.96 (m, 1H)

### Compound AAV: 1-(2-chloro-6-hydroxyphenyl)-3-((2-(2,6-dioxopiperidin-3-yl)-1-oxoisoindolin-5-yl)methyl)urea

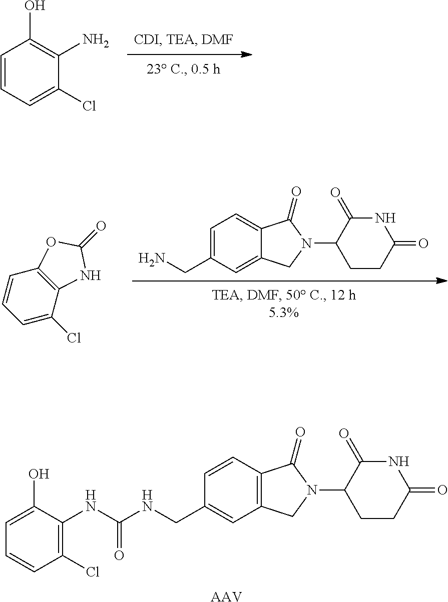 [SMILES: Nc1c(O)cccc1Cl ; O=c1[nH]c2c(Cl)cccc2o1 ; NCc1ccc2c(c1)CN(C1CCC(=O)NC1=O)C2=O ; O=C1CCC(N2Cc3cc(CNC(=O)Nc4c(O)cccc4Cl)ccc3C2=O)C(=O)N1 ; "AAV"]

Step 1. To a solution of 2-amino-3-chloro-phenol (50 mg, 348 umol, 1.0 equiv) in DMF (1 mL) was added CDI (67 mg, 417 umol, 1.2 equiv) and TEA (105 mg, 1.0 mmol, 145 μL, 3.0 equiv). The mixture was stirred at 23° C. for 0.5 h. The mixture was filtered and was concentrated to give the residue and used for next step without further purification. Compound 4-chloro-3H-1,3-benzoxazol-2-one (59 mg, 347.95 umol) was obtained as a colorless oil.

Step 2. To a solution of 4-chloro-3H-1,3-benzoxazol-2-one (59. mg, 348 umol, 1.0 equiv) and 3-[5-(aminomethyl)-1-oxo-isoindolin-2-yl]piperidine-2,6-dione (97 mg, 313 umol, 0.9 equiv, HCl) in DMF (2 mL) was added TEA (70 mg, 696 umol, 96 μL, 2.0 equiv) was stirred at 50° C. for 12 h. The mixture was filtered and was concentrated to get the residue. The residue was purified by prep-HPLC (HCL condition; column: Phenomenex luna C18 150*25 mm*10 um; mobile phase: [water(FA)-ACN]; B %: 27%-47%, 10 min) to give the 1-(2-chloro-6-hydroxy-phenyl)-3-[[2-(2,6-dioxo-3-piperidyl)-1-oxo-isoindolin-5-yl]methyl]urea (8.4 mg, 18 umol, 5% yield, 97% purity) as a yellow solid. ¹H NMR: (400 MHz, DMSO-d₆) δ=11.16-10.75 (m, 1H), 10.15-9.73 (m, 1H), 8.44-8.32 (m, 1H), 7.91 (s, 1H), 7.77-7.64 (m, 1H), 7.54 (s, 1H), 7.46 (d, J=7.5 Hz, 1H), 7.29-7.20 (m, 1H), 7.08-6.99 (m, 1H), 6.95-6.89 (m, 1H), 6.85-6.80 (m, 1H), 5.16-5.05 (m, 1H), 4.54-4.24 (m, 4H), 3.01-2.83 (m, 1H), 2.69-2.64 (m, 1H), 2.36-2.30 (m, 1H), 2.05-1.95 (m, 2H)

### Compound AAW: 1-(4-chloro-3-hydroxyphenyl)-3-((2-(2,6-dioxopiperidin-3-yl)-1-oxoisoindolin-5-yl)methyl)urea

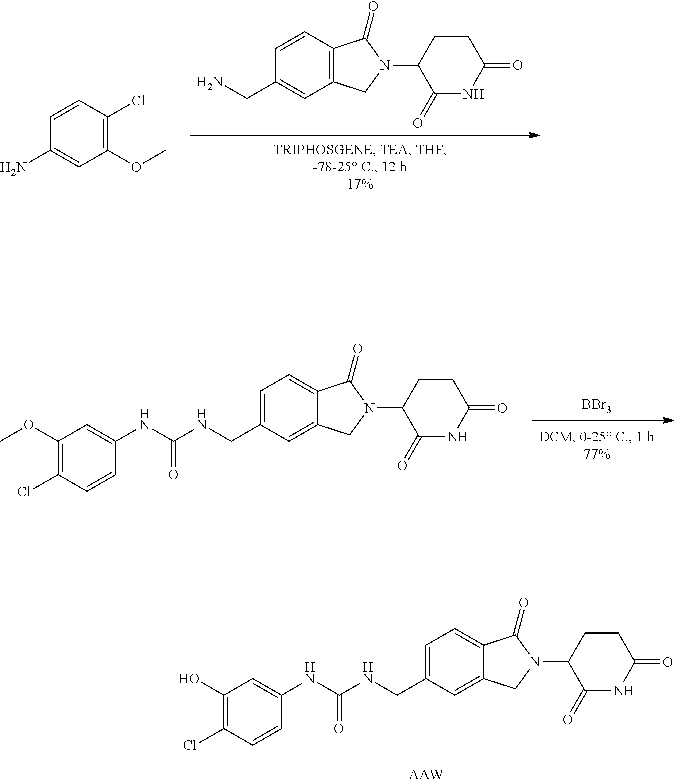

Step 1. To a solution of triphosgene (750 mg, 2.53 mmol, 0.8 equiv) in THF (30 mL) was added 4-chloro-3-methoxy-aniline (500 mg, 3.17 mmol, 1 equiv), TEA (3.21 g, 31.7 mmol, 4.42 mL, 10 equiv) in THF (10 mL) slowly at −78° C., and then it was stirred for 0.5 h. 3-[5-(aminomethyl)-1-oxo-isoindolin-2-yl]piperidine-2,6-dione (1.41 g, 3.17 mmol, 1 equiv, p-TSA) was added to the mixture and then it was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Phenomenex luna C18 150*25 mm*10 um; mobile phase: [water(FA)-ACN]; B %: 18%-48%, 2 min) to give 1-(4-chloro-3-methoxy-phenyl)-3-[[2-(2,6-dioxo-3-piperidyl)-1-oxo-isoindolin-5-yl]methyl]urea (250 mg, 547 umol, 17% yield) as a yellow solid.

Step 2. To a solution of 1-(4-chloro-3-methoxy-phenyl)-3-[[2-(2,6-dioxo-3-piperidyl)-1-oxo-isoindolin-5-yl]methyl]urea (20 mg, 43.8 umol, 1 equiv) in DCM (2 mL) was added BBr₃ (54.8 mg, 218 umol, 21.09 uL, 5 equiv). The mixture was stirred at 0° C. for 2 h. The reaction mixture was quenched by addition solvent H₂O 1 mL at 0° C., The reaction mixture was concentrated under reduced pressure to remove solvent. The residue was purified by prep-HPLC(column: Phenomenex C18 75*30 mm*3 um; mobile phase: [water(FA)-ACN]; B %: 15%-45%, 7 min) to give 1-(4-chloro-3-hydroxy-phenyl)-3-[[2-(2,6-dioxo-3-piperidyl)-1-oxo-isoindolin-5-yl]methyl]urea (15 mg, 33.8 umol, 77% yield) as a white solid. ¹H NMR (400 MHz, DMSO-d₆): δ 11.00 (s, 1H), 10.01 (s, 1H), 7.70 (d, J=7.8 Hz, 1H), 7.52 (s, 1H), 7.44 (d, J=8.1 Hz, 1H), 7.28 (d, J=2.2 Hz, 1H), 7.12 (d, J=8.7 Hz, 1H), 6.80-6.69 (m, 1H), 5.11 (d, J=5.0, 13.2 Hz, 1H), 4.45-4.24 (m, 4H), 3.00-2.81 (m, 1H), 2.62-2.55 (d, J=1.9, 15.6 Hz, 1H), 2.41-2.37 (m, 1H), 2.09-1.93 (m, 1H)

### Compound AAX: 1-((2-(2,6-dioxopiperidin-3-yl)-1-oxoisoindolin-5-yl)methyl)-3-(2-hydroxy-5-(trifluoromethyl)benzyl)urea

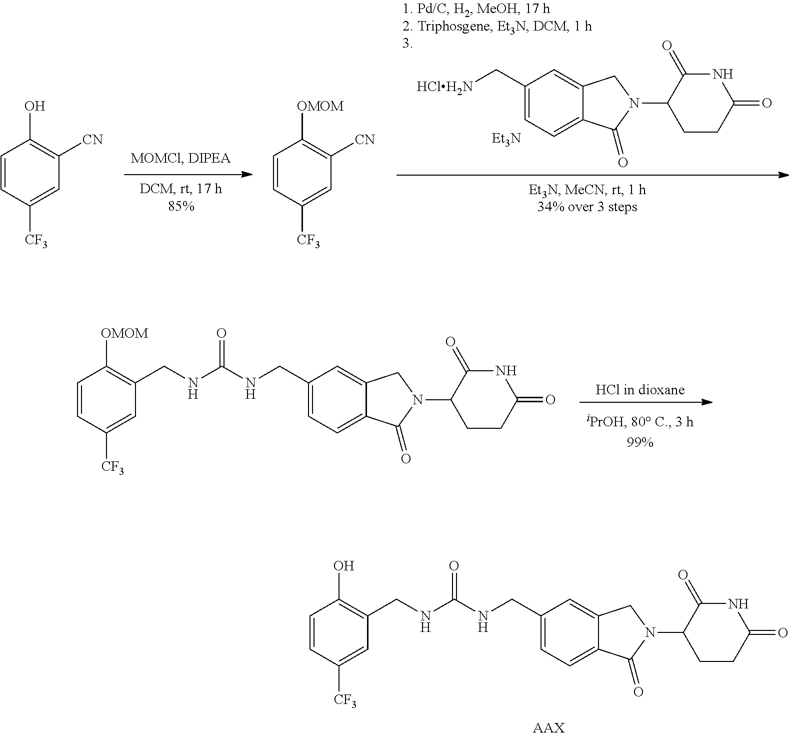

Step 1. To a solution of 2-hydroxy-5-trifluoromethylbenzonitrile (2.00 g, 10.7 mmol) and DIPEA (3.74 mL, 21.4 mmol) in DCM (100 mL) was added chloromethylmethylether (1.62 mL, 21.4 mmol) at rt under nitrogen. The reaction mixture was stirred at rt for 17 h and diluted with water (20 mL). The organic phase was separated, and the aqueous phase was extracted with DCM (3×20 mL). The combined organic phases were washed with water (2×20 mL) and brine (10 mL), dried over MgSO₄, filtered, and concentrated under reduced pressure. The crude product was purified by column chromatography on silica gel using a gradient of 0-100% EtOAc in hexane to provide 2-(methoxymethoxy)-5-(trifluoromethyl)benzonitrile as a solid (2.09 g, 85%).

Step 2. 1) A mixture of 2-(methoxymethoxy)-5-(trifluoromethyl)benzonitrile (2.08 g, 9.00 mmol) and 10% Pd/C (647 mg, 0.61 mmol) in MeOH (55 mL) was subjected to hydrogenation at 1 atm and rt for 17 h. The reaction mixture was filtered through Celite and the filtrate was concentrated under reduced pressure to provide the crude benzylamine as an oil. This intermediate was used in the next step without further purification.

Step 2. 2) A solution of the benzylamine intermediate obtained from step 1 in DCM (10 mL) was added to a solution of triphosgene (2.27 g, 7.65 mmol) in DCM (40 mL) at rt under nitrogen. The mixture was cooled to 0° C. and a solution of Et₃N (2.28 mL, 16.4 mmol) in DCM (4.0 mL) was added. The reaction mixture was stirred at rt for 1 h and concentrated under reduced pressure to afford the isocyanate intermediate which was used in the next step without further purification.

Step 2. 3) To a solution of the crude isocyanate intermediate obtained from step 2 and 3-(5-(aminomethyl)-1-oxoisoindolin-2-yl)piperidine-2,6-dione hydrochloride (2.37 g, 7.65 mmol) in MeCN (120 mL) was added Et₃N (2.13 mL, 15.3 mmol) at rt under nitrogen. The mixture was stirred at rt for 1 h and then diluted with water (30 mL) and EtOAc (35 mL). The organic phase was separated, and the aqueous phase was extracted with EtOAc (2×30 mL). The combined organic phases were dried over MgSO₄, filtered, and concentrated. The crude product was purified by column chromatography on silica gel using a gradient of 0-100% ⁱPrOH in CHCl₃ to provide 1-((2-(2,6-dioxopiperidin-3-yl)-1-oxoisoindolin-5-yl)methyl)-3-(2-(methoxymethoxy)-5-(trifluoromethyl)benzyl)urea as a solid (1.65 g, 34% over 3 steps).

Step 3. A mixture of 1-((2-(2,6-dioxopiperidin-3-yl)-1-oxoisoindolin-5-yl)methyl)-3-(2-(methoxymethoxy)-5-(trifluoromethyl)benzyl)urea (1.57 g, 2.94 mmol) and a solution of HCl in dioxane (4 M, 36.7 mL, 147 mmol) in a mixture of dioxane (100 mL) and ⁱPrOH (16 mL) was stirred at 80° C. for 1 h and then cooled to rt. The volatiles were removed under reduced pressure to provide 1-((2-(2,6-dioxopiperidin-3-yl)-1-oxoisoindolin-5-yl)methyl)-3-(2-hydroxy-5-(trifluoromethyl)benzyl)urea (1.43 g, 99%) as a solid. The crude material was used in the next step without further purification. Note: a small fraction was purified by semi-preparative HPLC (Gemini® 5 μm NX-C18 110 Å, 100×30 mm) using a gradient of 20%-100% MeCN in 10 mM ammonium formate to provide 1-((2-(2,6-dioxopiperidin-3-yl)-1-oxoisoindolin-5-yl)methyl)-3-(2-hydroxy-5-(trifluoromethyl)benzyl)urea with a 99% purity. ¹H NMR (DMSO-d₆, 400 MHz): δ_{H} 11.03-10.91 (1H, br s), 7.67-7.64 (1H, m), 7.44-7.37 (5H, m), 6.94 (1H, d, J=8.4 Hz), 6.84-6.78 (1H, m), 6.63-6.58 (1H, m), 5.14-5.07 (1H, m), 4.43-4.30 (4H, m), 4.20 (2H, d, J=6.0 Hz), 2.95-2.86 (1H, m), 2.64-2.56 (1H, m), 2.40-2.32 (1H, m), 2.01-1.94 (1H, m).

### Compound AAY: 1-((2-(2,6-dioxopiperidin-3-yl)-1-oxoisoindolin-5-yl)methyl)-3-(2-hydroxy-5-methoxyphenyl)urea

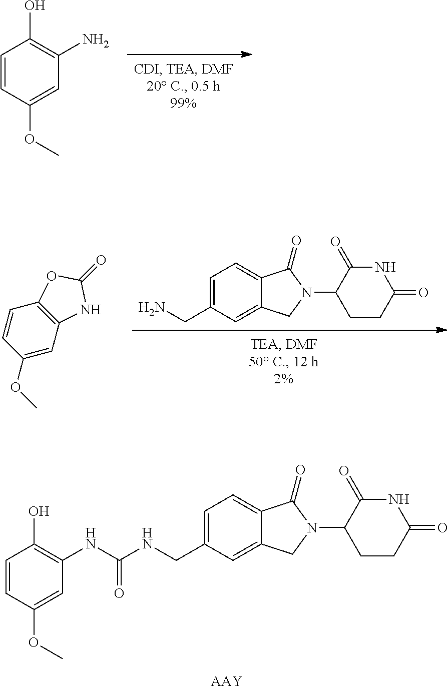

Step 1. To a solution of 2-amino-4-methoxy-phenol (100 mg, 718 umol, 1.0 equiv) in DMF (1 mL) was added TEA (218 mg, 2 mmol, 300 μL, 3.0 equiv), CDI (139 mg, 862 umol, 1.2 equiv). The mixture was stirred at 20° C. for 0.5 h. The reaction mixture was used into the next step without further purification. Compound 5-methoxy-3H-1,3-benzoxazol-2-one (118 mg, 718 umol, 99% yield) was obtained as a yellow oil.

Step 2. To a solution of 5-methoxy-3H-1,3-benzoxazol-2-one (118 mg, 718 umol, 1.0 equiv) in DMF (2 mL) was added TEA (145 mg, 1 mmol, 199 μL, 2.0 equiv) and 3-[5-(aminomethyl)-1-oxo-isoindolin-2-yl]piperidine-2,6-dione (200 mg, 646 umol, 0.9 equiv, HCl) . The mixture was stirred at 50° C. for 12 h. The mixture was concentrated to give a residue. The residue was purified by prep-HPLC (column: Phenomenex luna C18 150*25 mm*10 um; mobile phase: [water(FA)-ACN]; B %: 13%-43%, 10 min). Compound 1-[[2-(2,6-dioxo-3-piperidyl)-1-oxo-isoindolin-5-yl]methyl]-3-(2-hydroxy-5-methoxy-phenyl)urea (5.92 mg, 12 umol, 2% yield, 95% purity) was obtained as an off-white solid. ¹H NMR (400 MHz, DMSO-d₆) δ=11.09-10.84 (m, 1H), 9.50 (s, 1H), 8.44 (s, 1H), 8.13 (s, 1H), 7.72-7.63 (m, 2H), 7.58-7.48 (m, 2H), 7.43 (d, J=8.0 Hz, 1H), 6.69 (d, J=8.8 Hz, 1H), 6.31 (dd, J=3.2, 8.8 Hz, 1H), 5.10 (m, 1H), 4.50-4.28 (m, 4H), 3.62 (s, 3H), 2.95-2.84 (m, 1H), 2.60 (m, 1H), 2.41-2.32 (m, 1H), 2.04-1.95 (m, 1H).

### Compound AAZ: 2-((5-chloro-2-(3-((2-(2,6-dioxopiperidin-3-yl)-4-fluoro-1-oxoisoindolin-5-yl)methyl)ureido)-4-methoxyphenoxy)methyl)acrylic acid

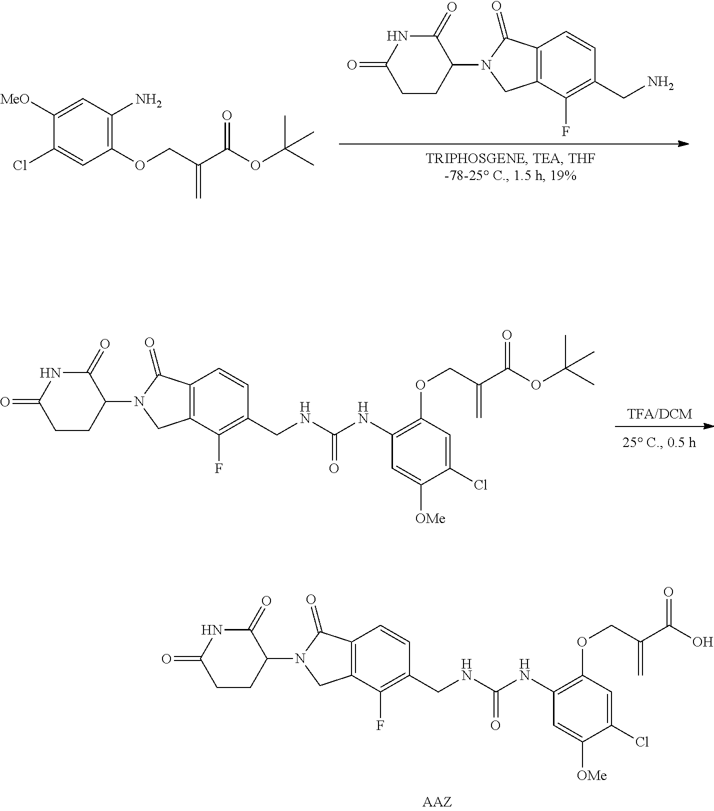 [SMILES: C=C(COc1cc(Cl)c(OC)cc1N)C(=O)OC(C)(C)C ; NCc1ccc2c(c1F)CN(C1CCC(=O)NC1=O)C2=O ; C=C(COc1cc(Cl)c(OC)cc1NC(=O)NCc1ccc2c(c1F)CN(C1CCC(=O)NC1=O)C2=O)C(=O)OC(C)(C)C ; "TFA/DCM" ; C=C(COc1cc(Cl)c(OC)cc1NC(=O)NCc1ccc2c(c1F)CN(C1CCC(=O)NC1=O)C2=O)C(=O)O]

Step 1. To a solution of triphosgene (670 mg, 2.3 mmol, 1.8 equiv) in THF (40 mL) was added TEA (645 mg, 6.4 mmol, 887 μL, 5.0 equiv) and tert-butyl 2-[(2-amino-5-chloro-4-methoxy-phenoxy)methyl]prop-2-enoate (400 mg, 1.3 mmol, 1.0 equiv). The mixture was stirred at −78° C. for 0.5 h. Then3-[5-(aminomethyl)-4-fluoro-1-oxo-isoindolin-2-yl]piperidine-2,6-dione (417.8 mg, 1.3 mmol, 1.0 equiv, HCl) was added. The mixture was stirred at −78° C. for 0.5 h. The mixture was stirred at 25° C. for 0.5 h. The residue was purified by prep-TLC (SiO₂, Petroleum ether: Ethyl acetate=0:1) to give a compound tert-butyl 2-[[5-chloro-2-[[2-(2,6-dioxo-3-piperidyl)-4-fluoro-1-oxo-isoindolin-5-yl] methylcarbamoylamino]-4-methoxy-phenoxy] methyl]prop-2-enoate (0.15 g, 19% yield) as a colorless oil.

Step 2. To a solution of tert-butyl 2-[[5-chloro-2-[[2-(2,6-dioxo-3-piperidyl)-4-fluoro-1-oxo-isoindolin-5-yl] methylcarbamoylamino]-4-methoxy-phenoxy] methyl]prop-2-enoate (80 mg, 126.8 umol, 1.0 equiv) in DCM/TFA=1:1 (126.8 umol, 1 mL, 1 equiv). The mixture was stirred at 25° C. for 0.5 h. The reaction mixture was concentrated under reduced pressure to give 2-[[5-chloro-2-[[2-(2,6-dioxo-3-piperidyl)-4-fluoro-1-oxo-isoindolin-5-yl]methylcarbamoylamino]-4-methoxy-phenoxy] methyl]prop-2-enoic acid (87 mg, 99% yield, TFA salt) as a colorless oil.

### Compound ABA: 2-((2-(3-((2-(2,6-dioxopiperidin-3-yl)-4-fluoro-1-oxoisoindolin-5-yl)methyl)ureido)-5-(trifluoromethyl)phenoxy)methyl)acrylic acid

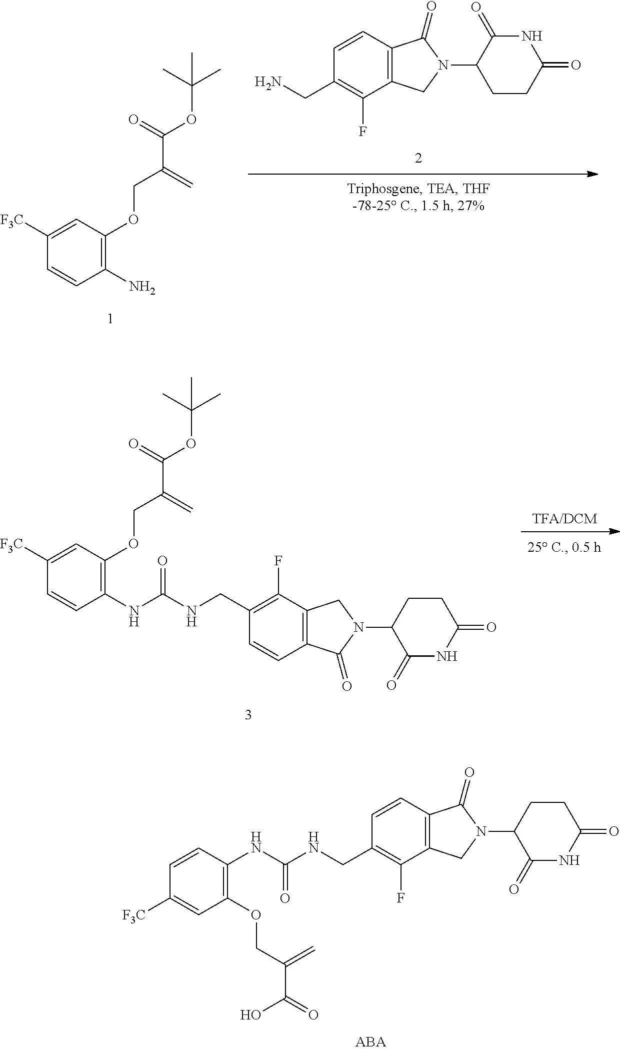 [SMILES: C=C(COc1cc(C(F)(F)F)ccc1N)C(=O)OC(C)(C)C ; NCc1ccc2c(c1F)CN(C1CCC(=O)NC1=O)C2=O ; C=C(COc1cc(C(F)(F)F)ccc1NC(=O)NCc1ccc2c(c1F)CN(C1CCC(=O)NC1=O)C2=O)C(=O)OC(C)(C)C ; "TFA/DCM" ; C=C(COc1cc(C(F)(F)F)ccc1NC(=O)NCc1ccc2c(c1F)CN(C1CCC(=O)NC1=O)C2=O)C(=O)O]

Step 1. To a solution of triphosgene (590 mg, 1.9 mmol, 2.1 equiv) in THF (15 mL) was added Et₃N (478.36 mg, 4.7 mmol, 658 μL, 5.0 equiv) and tert-butyl 2-[[2-amino-5-(trifluoromethyl)phenoxy]methyl]prop-2-enoate (300 mg, 945.4 umol, 1.0 equiv). The mixture was stirred at −78° C. for 0.5 h. Then 3-[5-(aminomethyl)-4-fluoro-1-oxo-isoindolin-2-yl]piperidine-2,6-dione (309.8 mg, 945.4 umol, 1.0 equiv, HCl) was added. The mixture was stirred at −78° C. for 0.5 h. The mixture was stirred at 25° C. for 0.5 h. The reaction mixture was added 100 ml H₂O and extracted with EA (50 mL×3). The combined organic layers were filtered and concentrated under reduced pressure to give a residue. The residue was purified by prep-TLC (SiO₂, Petroleum ether: Ethyl acetate=0:1) to give a compound tert-butyl 2-[[2-[[2-(2,6-dioxo-3-piperidyl)-4-fluoro-1-oxo-isoindolin-5-yl] methylcarbamoylamino]-5-(trifluoromethyl) phenoxy] methyl]prop-2-enoate (0.16 g, 27% yield) as a colorless oil.

Step 2. To a solution of tert-butyl 2-[[2-[[2-(2,6-dioxo-3-piperidyl)-4-fluoro-1-oxo-isoindolin-5-yl] methylcarbamoylamino]-5-(trifluoromethyl) phenoxy] methyl]prop-2-enoate (50 mg, 78.7 umol, 1.0 equiv) in DCM (1.0 mL) and TFA (0.5 mL). The mixture was stirred at 25° C. for 0.5 h. The reaction mixture was concentrated under reduced pressure to give compound 2-[[2-[[2-(2,6-dioxo-3-piperidyl)-4-fluoro-1-oxo-isoindolin-5-yl]methylcarbamoylamino]-5-(trifluoromethyl) phenoxy] methyl]prop-2-enoic acid (53 mg, 97% yield, TFA salt) as a colorless oil.

### Compound ABB: 3-(2-(3-((2-(2,6-dioxopiperidin-3-yl)-1-oxoisoindolin-5-yl)methyl) ureido)-4,5-difluorophenoxy)-2-methylpropanoic acid

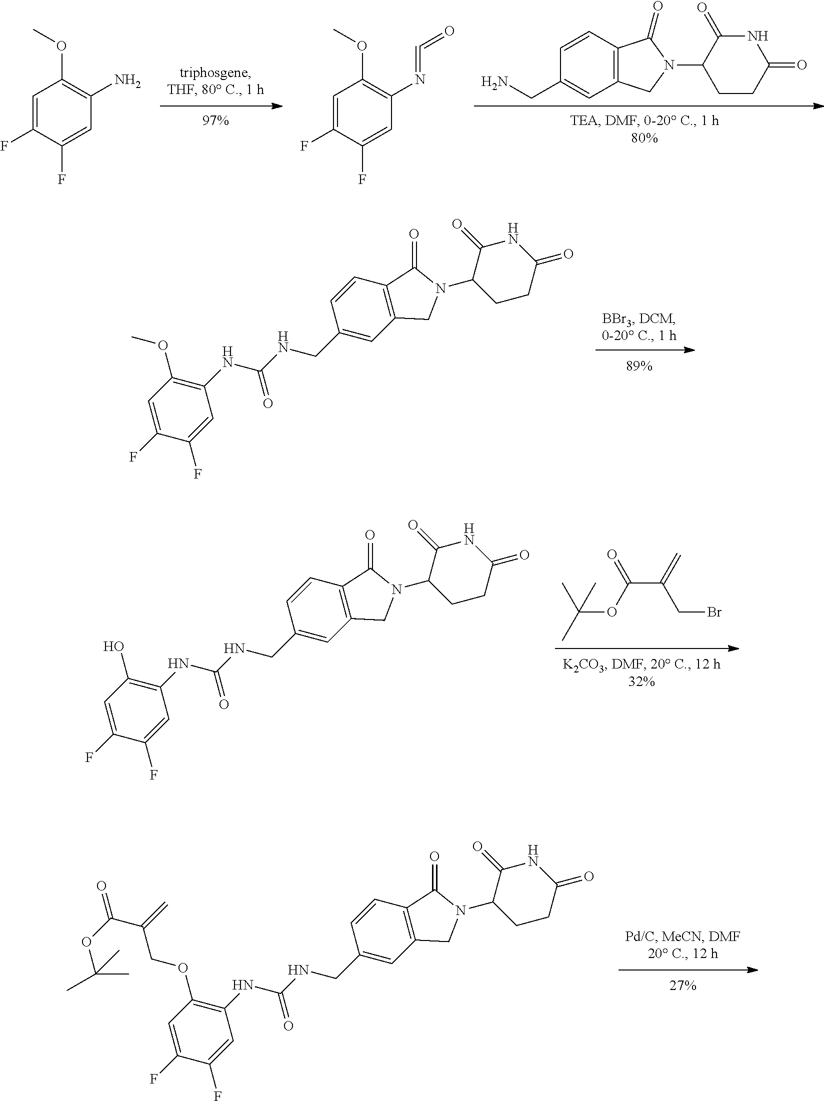

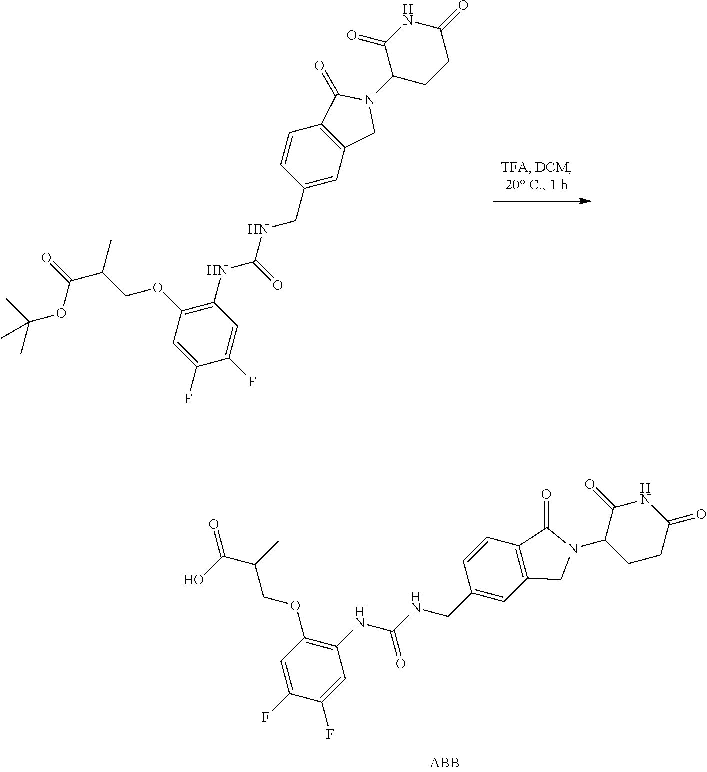 [SMILES: CC(COc1cc(F)c(F)cc1NC(=O)NCc1ccc2c(c1)CN(C1CCC(=O)NC1=O)C2=O)C(=O)OC(C)(C)C ; CC(COc1cc(F)c(F)cc1NC(=O)NCc1ccc2c(c1)CN(C1CCC(=O)NC1=O)C2=O)C(=O)O]

Step 1. To a solution of compound 4,5-difluoro-2-methoxyaniline (1.5 g, 9.43 mmol, 1.0 equiv) in THF (50 mL) was added triphosgene (2.82 g, 9.50 mmol, 1.0 equiv). The mixture was stirred at 80° C. for 1 h. The reaction mixture was concentrated under reduced pressure to give compound 1,2-difluoro-4-isocyanato-5-methoxybenzene (1.7 g, 9.18 mmol, 97% yield) as a brown oil.

Step 2. To a solution of compound 3-(5-(aminomethyl)-1-oxoisoindolin-2-yl)piperidine-2,6-dione (J. Am. Chem. Soc., 2017, 139, 15308)(3.0 g, 6.78 mmol, 70% purity, 1.0 equiv, HCl salt) in DMF (40 mL) was added TEA (6.86 g, 67.80 mmol, 9.4 mL, 10 equiv) and compound 1,2-difluoro-4-isocyanato-5-methoxybenzene (1.51 g, 8.14 mmol, 1.2 equiv) at 0° C. The mixture was stirred at 20° C. for 1 h. The reaction mixture was partitioned between H₂O (200 mL) and EtOAc (100 mL). The organic phase was separated, washed with brine (100 mL), dried over Na₂SO₄, filtered and concentrated under reduced pressure to give a residue. The residue was purified by column chromatography (SiO₂, Petroleum ether/Ethyl acetate=1/1 to Ethyl acetate/Methanol=10//1). Compound 1-(4,5-difluoro-2-methoxyphenyl)-3-((2-(2,6-dioxopiperidin-3-yl)-1-oxoisoindolin-5-yl)methyl)urea (2.5 g, 5.45 mmol, 80% yield) was obtained as a brown solid.

Step 3. To a solution of compound 1-(4,5-difluoro-2-methoxyphenyl)-3-((2-(2,6-dioxopiperidin-3-yl)-1-oxoisoindolin-5-yl)methyl)urea (1.5 g, 3.27 mmol, 1.0 equiv) in DCM (10 mL) was added BBr₃ (4.1 g, 16.36 mmol, 1.58 mL, 5.0 equiv) at 0° C. The mixture was stirred at 20° C. for 1 h. The reaction mixture was quenched by addition H₂O (5 mL) at 0° C., and then filtered to give compound 1-(4,5-difluoro-2-hydroxyphenyl)-3-((2-(2,6-dioxopiperidin-3-yl)-1-oxoisoindolin-5-yl)methyl)urea (1.3 g, 2.93 mmol, 89% yield) as a brown solid.

Step 4. To a solution of compound 1-(4,5-difluoro-2-hydroxyphenyl)-3-((2-(2,6-dioxopiperidin-3-yl)-1-oxoisoindolin-5-yl)methyl)urea (1.3 g, 2.93 mmol, 1.0 equiv) and compound tert-butyl 2-(bromomethyl)acrylate (646 mg, 2.93 mmol, 1.0 equiv) in DMF (10 mL) was added K₂CO₃ (808 mg, 5.85 mmol, 2.0 equiv). The mixture was stirred at 20° C. for 12 h. The reaction mixture was filtered and concentrated under reduced pressure to give a residue. The residue was purified by prep-HPLC (column: Waters Xbridge C18 150*50 mm*10 um; mobile phase: [water (NH₄HCO₃)-ACN]; B %: 34%-64%, 9 min) to give desired compound tert-butyl 2-((2-(3-((2-(2,6-dioxopiperidin-3-yl)-1-oxoisoindolin-5-yl)methyl)ureido)-4,5-difluorophenoxy)methyl)acrylate (550 mg, 940 umol, 32% yield) as a pink solid.

Step 5. To a solution of compound tert-butyl 2-((2-(3-((2-(2,6-dioxopiperidin-3-yl)-1-oxoisoindolin-5-yl)methyl)ureido)-4,5-difluorophenoxy)methyl)acrylate (550 mg, 940 umol, 1.0 equiv) in MeCN (20 mL) and DMF (5 mL) was added Pd/C (100 mg, 940 umol, 10% purity, 1.0 equiv) under N₂ atmosphere. The suspension was degassed and purged with H₂ for 3 times. The mixture was stirred under H₂ (15 Psi) at 20° C. for 12 h. The reaction mixture was filtered and concentrated under reduced pressure to give a residue. The residue was purified by prep-HPLC (column: Phenomenex luna C18 150*40 mm*15 um; mobile phase: [water (TFA)-ACN]; B %: 33%-63%, 10 min). Compound tert-butyl 3-(2-(3-((2-(2,6-dioxopiperidin-3-yl)-1-oxoisoindolin-5-yl)methyl)ureido)-4,5-difluorophenoxy)-2-methylpropanoate(150 mg, 255.72 umol, 27.18% yield) was obtained as a white solid.

Step 6. To a solution of compound tert-butyl 3-(2-(3-((2-(2,6-dioxopiperidin-3-yl)-1-oxoisoindolin-5-yl)methyl)ureido)-4,5-difluorophenoxy)-2-methylpropanoate (150 mg, 255 umol, 1.0 equiv) in DCM (1 mL) was added TFA (0.5 mL). The mixture was stirred at 20° C. for 1 h. The reaction mixture was concentrated under reduced pressure to give compound 3-(2-(3-((2-(2,6-dioxopiperidin-3-yl)-1-oxoisoindolin-5-yl)methyl)ureido)-4,5-difluorophenoxy)-2-methylpropanoic acid (135 mg, 254 umol, 99% yield) as a yellow gum.

### Compound ABC: 1-((2-(2,6-dioxopiperidin-3-yl)-1-oxoisoindolin-5-yl)methyl)-3-(2-hydroxy-5-methoxybenzyl)urea

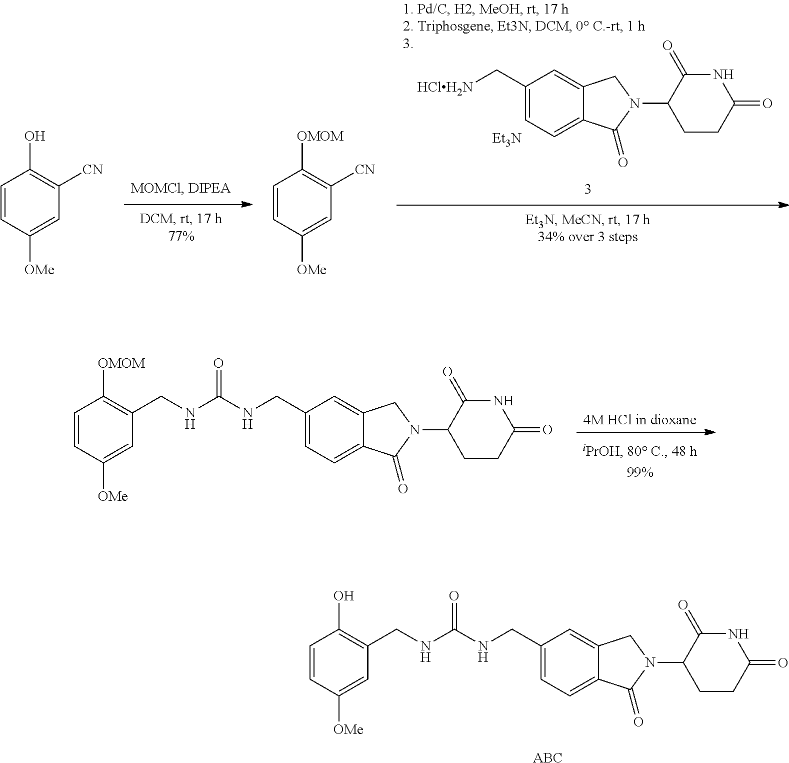

Step 1. To a solution of 2-hydroxy-5-methoxybenzonitrile (1.00 g, 6.70 mmol) and DIPEA (2.35 mL, 13.4 mmol) in DCM (50 mL) was added chloromethylmethylether (1.02 mL, 13.4 mmol) at rt under nitrogen. The reaction mixture was stirred at rt for 17 h and then diluted with water (20 mL). The organic phase was separated, and the aqueous phase was extracted with DCM (3×20 mL). The combined organic phases were washed with water (2×20 mL) and brine (10 mL), dried over MgSO₄, filtered, and concentrated under reduced pressure. The crude product was purified by column chromatography on silica gel using a gradient of 0-100% EtOAc in hexane to provide the title compound 5-methoxy-2-(methoxymethoxy)benzonitrile (995 mg, 77%) as a solid. ¹H NMR (DMSO-d₆, 400 MHz): δ_{H} 7.33 (1H, d, J=2.9 Hz), 7.28-7.22 (2H, m), 5.28 (2H, s), 3.75 (3H, s), 3.42 (3H, s).

Step 2. 1) A mixture of 5-methoxy-2-(methoxymethoxy)benzonitrile (809 mg, 4.19 mmol) and 10 wt % Pd/C (356 mg, 0.335 mmol) in MeOH (90 mL) was subjected to hydrogenation at 1 atm and rt for 17 h. The reaction mixture was filtered through Celite and the filtrate was concentrated under reduced pressure to provide the crude benzylamine as an oil. This intermediate was used in the next step without further purification.

Step 2. 2) A solution of the benzylamine intermediate obtained from step 1 in DCM (10 mL) was added to a solution of triphosgene (869 mg, 2.93 mmol) in DCM (30 mL) at rt under nitrogen. The mixture was cooled to 0° C. and a solution of Et₃N (0.874 mL, 6.27 mmol) in DCM (4.0 mL) was added. The reaction mixture was stirred at rt for 1 h and then concentrated under reduced pressure to afford the crude isocyanate which was used in the next step without further purification.

Step 2. 3) To a solution of the isocyanate intermediate obtained from step 2 and 3-(5-(aminomethyl)-1-oxoisoindolin-2-yl)piperidine-2,6-dione hydrochloride (908 mg, 2.93 mmol) in MeCN (30 mL) was added Et₃N (0.817 mL, 5.86 mmol) at rt under nitrogen. The reaction mixture was stirred at rt for 17 h. Water (30 mL), EtOAc (35 mL) and an aqueous solution of HCl (1 M, 10 mL) were sequentially added. The organic phase was separated, and the aqueous phase was extracted with EtOAc (2×25 mL). The combined organic phases were dried over MgSO₄, filtered, and concentrated under reduced pressure. The crude product was purified by column chromatography on silica gel using a gradient of 0-100% ⁱPrOH in CHCl₃ to provide 1-((2-(2,6-dioxopiperidin-3-yl)-1-oxoisoindolin-5-yl)methyl)-3-(5-methoxy-2-(methoxymethoxy)benzyl)urea (440 mg, 30%) as a solid.

Step 3. A mixture of 1-((2-(2,6-dioxopiperidin-3-yl)-1-oxoisoindolin-5-yl)methyl)-3-(5-methoxy-2-(methoxymethoxy)benzyl)urea (329 mg, 0.663 mmol) and a solution of HCl in dioxane (4 M, 8.28 mL, 33.1 mmol) in a mixture of dioxane (50 mL) and ⁱPrOH (4.0 mL) was stirred at 80° C. for 48 h and then cooled to rt. The volatiles were evaporated under reduced pressure and the residual material was lyophilized to provide 1-((2-(2,6-dioxopiperidin-3-yl)-1-oxoisoindolin-5-yl)methyl)-3-(2-hydroxy-5-methoxybenzyl)urea as a solid (298 mg, 99%). The crude phenol 1-((2-(2,6-dioxopiperidin-3-yl)-1-oxoisoindolin-5-yl)methyl)-3-(2-hydroxy-5-methoxybenzyl)urea was used in the next step without further purification. Note: a small fraction was purified by semi-preparative HPLC (Gemini® 5 μm NX-C18 110 Å, 100×30 mm) using a gradient of 40-100% MeCN in 10 mM ammonium formate to provide 1-((2-(2,6-dioxopiperidin-3-yl)-1-oxoisoindolin-5-yl)methyl)-3-(2-hydroxy-5-methoxybenzyl)urea with a 99% purity. ¹H NMR (DMSO-d₆, 400 MHz): δ_{H} 2.01-1.96 (1H, m), 2.43-2.31 (1H, m), 2.68-2.58 (1H, m), 2.95-2.86 (1H, m), 3.63 (3H, s), 4.12 (2H, d, J=6.0 Hz), 4.36-4.28 (3H, m), 4.42 (1H, d, J=17.2 Hz), 5.10 (1H, dd, J=13.3, 5.1 Hz), 6.51 (1H, t, J=6.0 Hz), 6.71-6.63 (3H, m), 6.76 (1H, t, J=6.1 Hz), 7.39 (1H, d, J=7.9 Hz), 7.46 (1H, s), 7.66 (1H, d, J=7.8 Hz), 9.28 (1H, s), 10.93 (1H, br s).

### Compound ABD: 1-(4-chloro-3-hydroxyphenyl)-3-((2-(2,6-dioxopiperidin-3-yl)-4-fluoro-1-oxoisoindolin-5-yl)methyl)urea

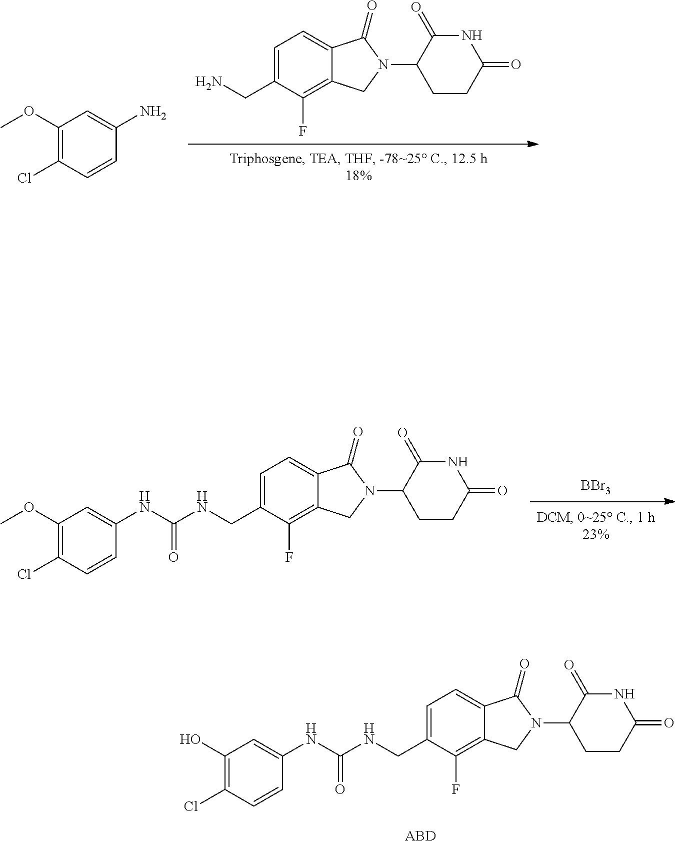

Step 1. To a solution of triphosgene (0.92 g, 3.1 mmol, 0.98 equiv) in THF (30 mL) was added 4-chloro-3-methoxyaniline (0.50 g, 3.2 mmol, 1.0 equiv) and TEA (3.2 g, 32 mmol, 4.4 mL, 10 equiv) in THF (10 mL) slowly at −78° C., and then it was stirred for 0.5 h. 3-(5-(aminomethyl)-4-fluoro-1-oxoisoindolin-2-yl)piperidine-2,6-dione (J. Am. Chem. Soc., 2017, 139, 15308)(1.0 g, 3.2 mmol, 1.0 equiv, HCl salt) was added to the mixture and then it was stirred at 20° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Phenomenex luna C18 150*25 mm*10 um; mobile phase: [water(FA)-ACN]; B %: 22%-52%, 10 min) to afford 1-(4-chloro-3-methoxyphenyl)-3-((2-(2,6-dioxopiperidin-3-yl)-4-fluoro-1-oxoisoindolin-5-yl)methyl)urea (0.28 mg, 0.56 umol, 18% yield) as a white solid.

Step 2. To a solution of compound 1-(4-chloro-3-methoxyphenyl)-3-((2-(2,6-dioxopiperidin-3-yl)-4-fluoro-1-oxoisoindolin-5-yl)methyl)urea (180 mg, 379 umol, 1.0 equiv) in DCM (2 mL) was added BBr₃ (1.42 g, 5.69 mmol, 548 μL, 15.0 equiv) at 0° C. The mixture was stirred at 20° C. for 1 h. The reaction mixture was quenched by addition H₂O (5 mL) at 0° C., the reaction mixture was concentrated under reduced pressure to remove DCM. The residue was filtered to afford crude product. The residue was triturated by THF (1 mL) to afford compound ABD (41 mg, 88 umol, 23% yield) as a white solid. ¹H NMR: (400 MHz, DMSO-d₆): 6.23 (s, 1H), 5.52-4.93 (m, 1H), 4.10-3.60 (m, 1H), 2.89-2.73 (m, 2H), 2.42-2.26 (m, 1H), 2.01-1.82 (m, 2H), 0.45-0.19 (m, 1H), -0.07-1-0.25 (m, 1H), -0.31-1-0.46 (m, 3H), -1.75-1-1.96 (m, 1H), -2.07-1-2.18 (m, 1H), -2.65-1-2.87 (m, 2H)

### Compound ABE: 1-(2-chloro-5-hydroxyphenyl)-3-((2-(2,6-dioxopiperidin-3-yl)-1-oxoisoindolin-5-yl)methyl)urea

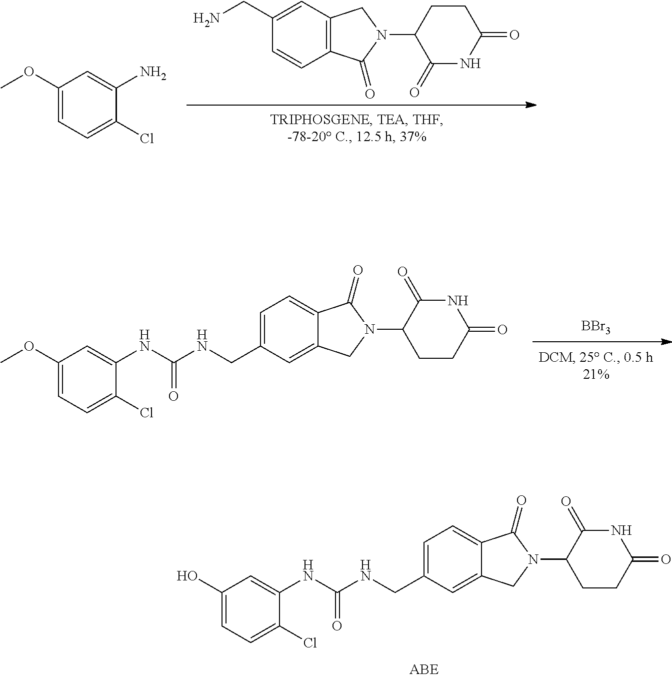

Step 1. To a solution of TEA (3.21 g, 31.7 mmol, 10.0 equiv) and triphosgene (1.09 g, 3.67 mmol, 1.2 equiv) in THF (8 mL) was added 2-chloro-5-methoxy-aniline (500 mg, 3.17 mmol, 1.0 equiv) in THF (2 mL) at −78° C., and then it was stirred for 0.5 h. Then 3-[5-(aminomethyl)-1-oxo-isoindolin-2-yl]piperidine-2,6-dione (J. Am. Chem. Soc., 2017, 139, 15308)(867.04 mg, 3.17 mmol, 1.0 equiv) was added to the mixture and then it was slowly warmed to 20° C. and stirred for 12 h. The residue was diluted with H₂O (100 mL), extracted with EA (100 mL×3) and washed with H₂O (50 mL×2). The organic phase was concentrated to afford crude product The residue was purified by prep-HPLC (column: Phenomenex luna C18 150*40 mm*15 um; mobile phase: [water(TFA)-ACN]; B %: 20%-50%, 10 min) to afford 1-(2-chloro-5-methoxy-phenyl)-3-[[2-(2,6-dioxo-3-piperidyl)-1-oxo-isoindolin-5-yl]methyl]urea (100 mg, 219 μmol, 7% yield) as a yellow solid.

Step 2. To a solution of 1-(2-chloro-5-methoxy-phenyl)-3-[[2-(2,6-dioxo-3-piperidyl)-1-oxo-isoindolin-5-yl]methyl]urea (100 mg, 219 μmol, 1.0 equiv) in DCM (3 mL) was added BBr₃ (548 mg, 2.19 mmol, 10.0 equiv). The mixture was stirred at 25° C. for 0.5 h. This reaction was quenched by cold H₂O (1 mL), The residue was extracted with EA (50 mL×3) and washed with H₂O (20 mL*2). The organic phase was concentrated to afford crude product The residue was purified by prep-HPLC (column: 3_Phenomenex Luna C18 75*30 mm*3 um; mobile phase: [water(TFA)-ACN]; B %: 21%-41%, 8 min) to afford 1-(2-chloro-5-hydroxy-phenyl)-3-[[2-(2,6-dioxo-3-piperidyl)-1-oxo-isoindolin-5-yl]methyl]urea (21.0 mg, 45.5 μmol, 21% yield, 96% purity) as a white solid. ¹H NMR (400 MHz, DMSO-d₆) δ 11.08-10.88 (m, 1H), 9.55-9.49 (m, 1H), 8.00 (s, 1H), 7.74-7.69 (m, 2H), 7.62 (m, 1H), 7.55-7.51 (m, 1H), 7.48-7.43 (m, 1H), 7.16-7.12 (m, 1H), 6.39-6.33 (m, 1H), 5.16-5.06 (m, 1H), 4.49-4.42 (m, 3H), 4.36-4.28 (m, 1H), 2.10-2.06 (m, 1H), 2.05-1.97 (m, 1H)

### Compound ABF: 1-(5-chloro-2-hydroxyphenyl)-3-((2-(2,6-dioxopiperidin-3-yl)-4-fluoro-1-oxoisoindolin-5-yl)methyl)urea

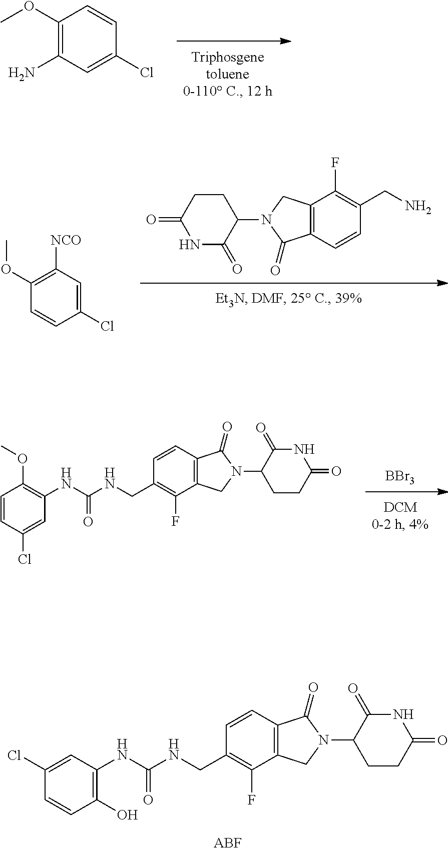

Step 1. To a solution of 5-chloro-2-methoxy-aniline (1 g, 6.3 mmol, 1.0 equiv) in toluene (10 mL) was added triphosgene (3.9 g, 13.1 mmol, 2.0 equiv) in toluene (10 mL) at 0° C. The mixture was stirred at 110° C. for 12 h. The reaction mixture was concentrated to afford 4-chloro-2-isocyanato-1-methoxy-benzene (1.1 g, 5.99 mmol) as a white solid.

Step 2. To a solution of 3-[5-(aminomethyl)-4-fluoro-1-oxo-isoindolin-2-yl] piperidine-2,6-dione (0.5 g, 1.5 mmol, 1.0 equiv, HCl salt) in DMF (10 mL) was added Et₃N (463.1 mg, 4.6 mmol, 637 μL, 3.0 equiv) and 4-chloro-2-isocyanato-1-methoxy-benzene (840 mg, 4.6 mmol, 3.0 equiv) at 0° C. The mixture was stirred at 25° C. for 12 h. The reaction mixture was added 50 mL H₂O and extracted with EA (50 mL×3). The combined organic layers were filtered and concentrated under reduced pressure to give a residue. The residue was purified by prep-TLC (SiO₂, DCM: MeOH=10:1) to give compound 1-(5-chloro-2-methoxy-phenyl)-3-[[2-(2,6-dioxo-3-piperidyl)-4-fluoro-1-oxo-isoindolin-5-yl] methyl] urea (0.2 g, 28% yield) as a white solid.

Step 3. To a solution of 1-(5-chloro-2-methoxy-phenyl)-3-[[2-(2,6-dioxo-3-piperidyl)-4-fluoro-1-oxo-isoindolin-5-yl] methyl] urea (160 mg, 336.9 umol, 1.0 equiv) in DCM (3 mL) was added BBr₃ (844.1 mg, 3.4 mmol, 324.6 uL, 10.0 equiv). The mixture was stirred at 0° C. for 2 h. After concentrated, the residue was purified by prep-HPLC (column: Phenomenex Synergi Polar-RP 100*25 mm*4 um; mobile phase: [water(TFA)-ACN]; B %: 35%-55%, 7 min) to afford 1-(5-chloro-2-hydroxy-phenyl)-3-[[2-(2,6-dioxo-3-piperidyl)-4-fluoro-1-oxo-isoindolin-5-yl]methyl]urea (7 mg, 4.0% yield) as a white solid. ¹H NMR (400 MHz, DMSO-d₆): δ 11.00 (s, 1H), 10.13 (s, 1H), 8.19 (s, 1H), 8.08 (d, J=2.08 Hz, 1H), 7.45-7.63 (m, 3H), 6.71-6.84 (m, 2H), 5.12 (m, 1H), 4.52-4.65 (m, 1H), 4.35-4.62 (m, 3H) 2.86-2.99 (m, 1H), 2.60 (d, J=16.87 Hz, 1H), 2.31-2.46 (m, 1H), 1.93-2.06 (m, 1H).

### Compound ABG: 2-((2-(3-((2-(2,6-dioxopiperidin-3-yl)-1-oxoisoindolin-5-yl)methyl)ureido)-5-(trifluoromethyl)phenoxy)methyl)acrylic acid

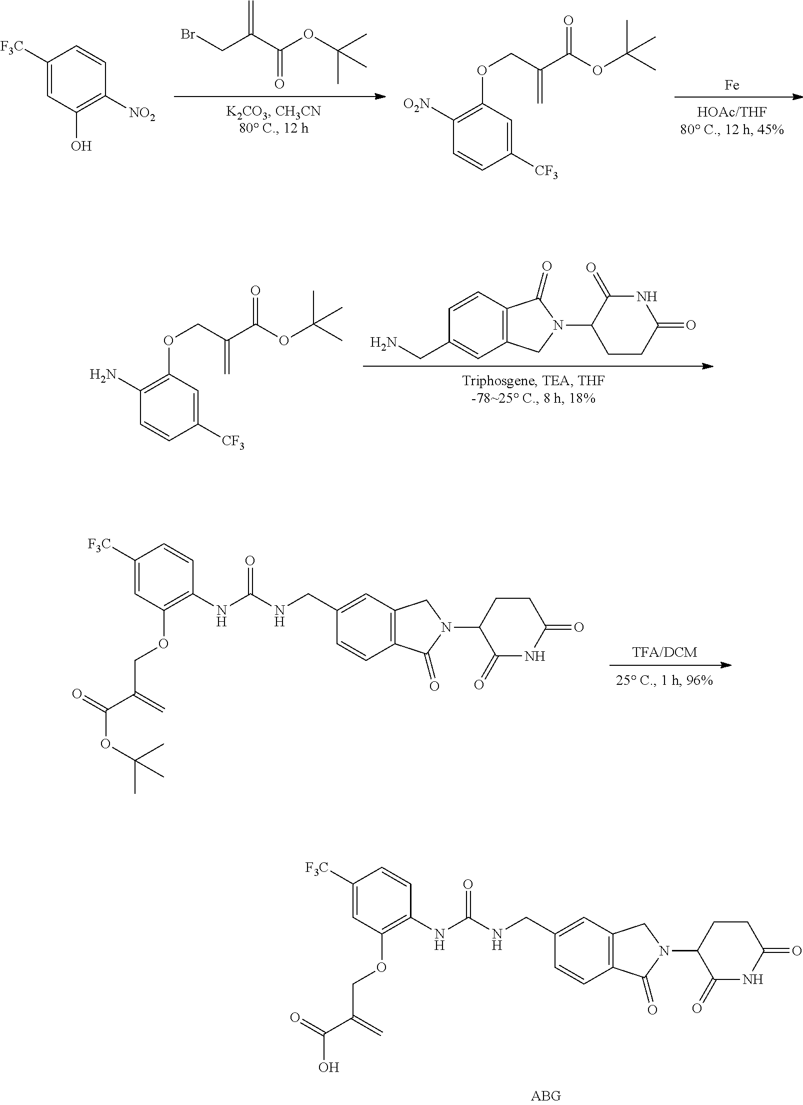

Step 1. To a solution of 2-nitro-5-(trifluoromethyl)phenol (2.50 g, 12.07 mmol, 1.0 equiv) in CH₃CN (30 mL) was added K₂CO₃ (5.00 g, 36.21 mmol, 3.0 equiv) and tert-butyl 2-(bromomethyl)prop-2-enoate (J. Med. Chem., 2021, 64, 1835)(2.67 g, 12.07 mmol, 1.0 equiv). The mixture was stirred at 80° C. for 12 h. The reaction mixture was filtered and concentrated under reduced pressure to give a residue. The residue was purified by column chromatography (SiO₂, Petroleum ether/Ethyl acetate=0/1) to give compound tert-butyl 2-((2-nitro-5-(trifluoromethyl)phenoxy)methyl)acrylate (4.1 g, crude) was obtained as a yellow oil.

Step 2. To a solution of compound tert-butyl 2-((2-nitro-5-(trifluoromethyl)phenoxy) methyl)acrylate (4.1 g, 11.81 mmol, 1.0 equiv) in THF (40 mL) was added Fe (3.30 g, 59.03 mmol, 5.0 equiv) and CH₃COOH (20 mL). The mixture was stirred at 50° C. for 12 h. The reaction was filtered and concentrated under reduced pressure to give a residue. The residue was purified by prep-HPLC (column: Phenomenex luna C18 (250*70 mm, 10 um); mobile phase: [water(FA)-MeOH]; B %: 50%-80%, 30 min) to give compound tert-butyl 2-((2-amino-5-(trifluoromethyl)phenoxy)methyl)acrylate (1.7 g, 5.36 mmol, 45% yield) was obtained as a yellow solid.

Step 3. To a solution of triphosgene (350.71 mg, 1.18 mmol, 0.8 equiv) in THF (50 mL) was added compound tert-butyl 2-((2-amino-5-(trifluoromethyl)phenoxy)methyl)acrylate (500 mg, 1.58 mmol, 1.0 equiv) and TEA (1.59 g, 15.76 mmol, 2.0 mL, 10.0 equiv) in THF (50 mL) at −78° C. for 0.5 h, and then 3-[5-(aminomethyl)-1-oxo-isoindolin-2-yl]piperidine-2,6-dione (J. Am. Chem. Soc., 2017, 139, 15308)(537 mg, 1.73 mmol, 1.1 equiv, HCl) was added, The mixture was stirred at 25° C. for 8 h. The reaction mixture was poured into 80 mL water and extracted with EA (3×30 mL). The organic layers were dried over anhydrous Na₂SO₄, filtered and concentrated to afford crude product. The residue was purified by prep-TLC (SiO₂, DCM: MeOH=10:1) to afford compound tert-butyl 2-((2-(3-((2-(2,6-dioxopiperidin-3-yl)-1-oxoisoindolin-5-yl)methyl)ureido)-5-(trifluoromethyl)phenoxy)methyl)acrylate (180 mg, 292 umol, 18% yield) as a white solid.

Step 4. To a solution of compound tert-butyl 2-((2-(3-((2-(2,6-dioxopiperidin-3-yl)-1-oxoisoindolin-5-yl)methyl)ureido)-5-(trifluoromethyl)phenoxy)methyl)acrylate (180 mg, 292 umol, 1.0 equiv) in DCM (2 mL) was added TFA (1 mL). The mixture was stirred at 25° C. for 1 h. The reaction mixture was filtered and concentrated under reduced pressure to give compound ABG (190 mg, 282 umol, 96% yield, TFA salt) as a red oil.

### Compound ABH: 1-((2-(2,6-dioxopiperidin-3-yl)-1-oxoisoindolin-5-yl)methyl)-3-(2-hydroxy-5-(trifluoromethyl)phenyl)urea

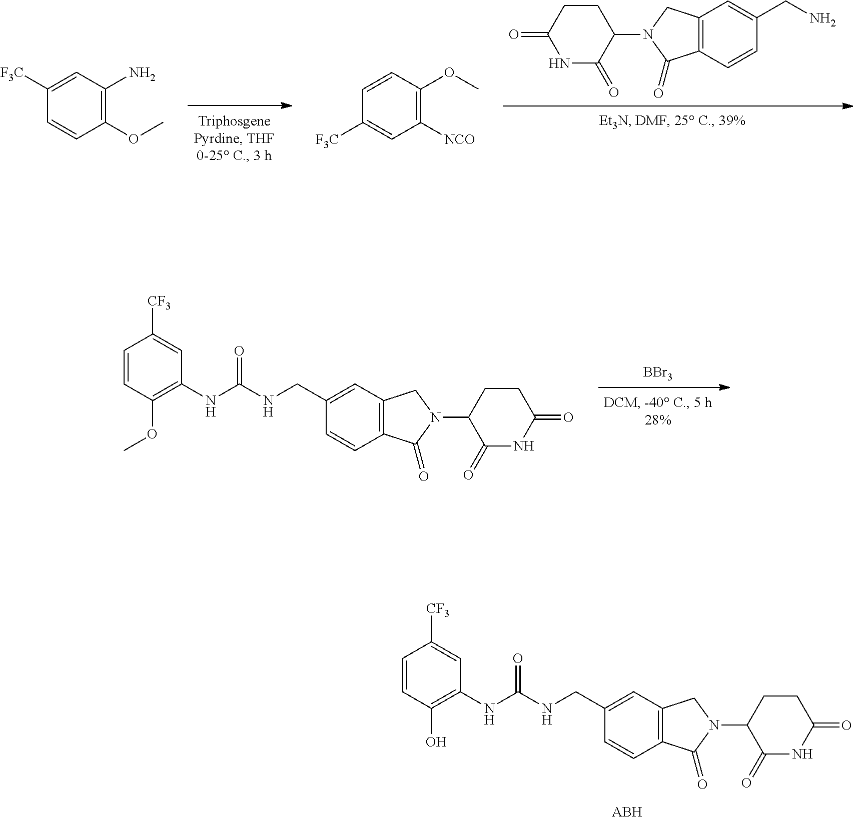

Step 1. To a solution of 2-methoxy-5-(trifluoromethyl)aniline (4 g, 21.0 mmol, 1.0 equiv), Pyrdine (4.9 g, 62.8 mmol, 5.1 mL, 3.0 equiv) in THF (30 mL) was added triphosgene (12.2 g, 41.1 mmol, 2.0 equiv) in THF (30 mL) at 0° C. The mixture was stirred at 25° C. for 3 h. The reaction mixture was concentrated to afford 2-isocyanato-1-methoxy-4-(trifluoromethyl)benzene (4.5 g, 99% yield) as a white solid.

Step 2. To a solution of 3-(5-(aminomethyl)-1-oxoisoindolin-2-yl)piperidine-2,6-dione (J. Am. Chem. Soc., 2017, 139, 15308)(1 g, 3.0 mmol, 1.0 equiv, HCl) in DMF (20 mL) was added Et₃N (2.4 g, 24.4 mmol, 3.4 mL, 8.0 equiv) and 2-isocyanato-1-methoxy-4-(trifluoromethyl)benzene (1.8 g, 8.2 mmol, 2.7 equiv). The mixture was stirred at 25° C. for 12 h. The residue was purified by prep-HPLC (column: Phenomenex luna C18 150*40 mm*15 um; mobile phase: [water(TFA)-ACN]; B %: 25%-55%, 10 min) to give a compound 1-[[2-(2,6-dioxo-3-piperidyl)-1-oxo-isoindolin-5-yl]methyl]-3-[2-methoxy-5-(trifluoromethyl)phenyl]urea (0.6 g, 39% yield) as a white solid.

Step 3. To a solution of 1-[[2-(2,6-dioxo-3-piperidyl)-1-oxo-isoindolin-5-yl]methyl]-3-[2-methoxy-5-(trifluoromethyl)phenyl]urea (0.55 g, 1.12 mmol, 1.0 equiv) in DCM (11 mL) was added BBr₃ (2.86 g, 11.42 mmol, 1.10 mL, 10.18 equiv) at −40° C., and then it was stirred for 5 h. The reaction mixture was slowly added to 100 mL sat. NaHCO₃, and then precipitate formed. After filtered, the filter cake was dried. The residue was purified by prep-HPLC (column: Phenomenex luna C18 150*40 mm*15 um; mobile phase: [water(TFA)-ACN]; B %: 20%-50%, 10 min) to afford 1-[[2-(2,6-dioxo-3-piperidyl)-1-oxo-isoindolin-5-yl]methyl]-3-[2-hydroxy-5-(trifluoromethyl)phenyl]urea (50 mg, 104.95 umol, 9.36% yield) as white solid.

### Compound ABI: 1-((2-(2,6-dioxopiperidin-3-yl)-4-fluoro-1-oxoisoindolin-5-yl)methyl)-3-(2-hydroxy-5-(trifluoromethyl)phenyl)urea

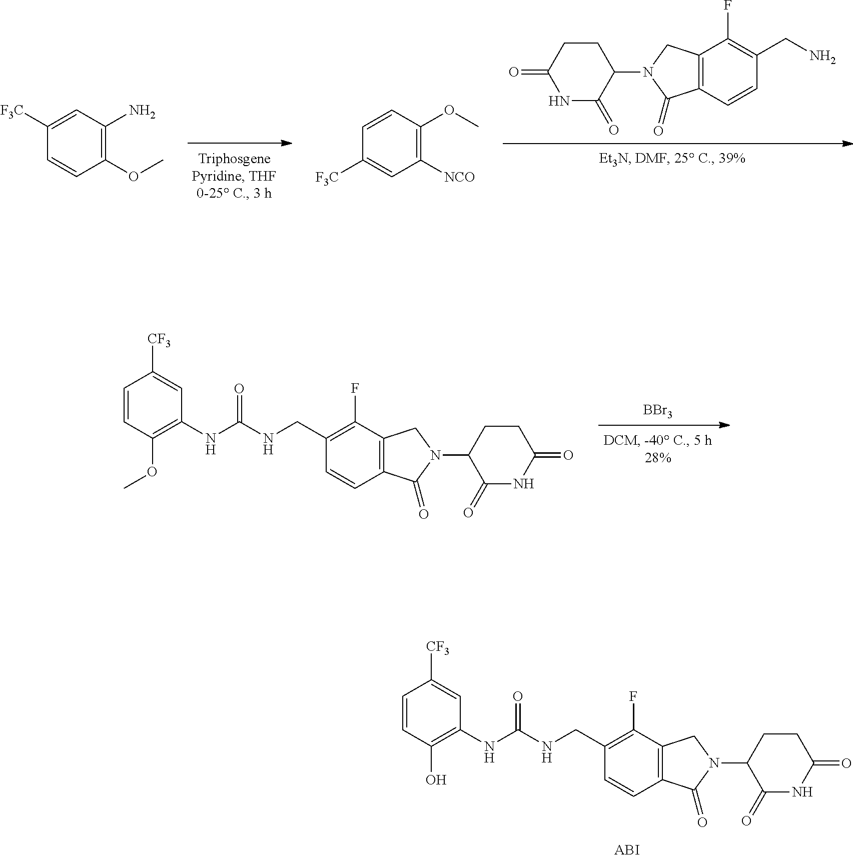

Step 1. To a solution of 2-methoxy-5-(trifluoromethyl)aniline (4 g, 21.0 mmol, 1.0 equiv), Pyridine (4.9 g, 62.8 mmol, 5.1 mL, 3.0 equiv) in THF (30 mL) was added triphosgene (12.2 g, 41.1 mmol, 2.0 equiv) in THF (30 mL) at 0° C. The mixture was stirred at 25° C. for 3 h. The reaction mixture was concentrated to afford 2-isocyanato-1-methoxy-4-(trifluoromethyl)benzene (4.5 g, 99% yield) as a white solid.

Step 2. To a solution of 3-[5-(aminomethyl)-4-fluoro-1-oxo-isoindolin-2-yl]piperidine-2,6-dione (1.0 g, 3.0 mmol, 1.0 equiv, HCl salt) in DMF (20 mL) was added Et₃N (2.4 g, 24.4 mmol, 3.4 mL, 8.0 equiv) and 2-isocyanato-1-methoxy-4-(trifluoromethyl)benzene (1.8 g, 8.2 mmol, 2.7 equiv). The mixture was stirred at 25° C. for 12 h. The residue was purified by prep-HPLC (column: Phenomenex luna C18 150*40 mm*15 um; mobile phase: [water(TFA)-ACN]; B %: 25%-55%, 10 min) to give a compound 1-[[2-(2,6-dioxo-3-piperidyl)-4-fluoro-1-oxo-isoindolin-5-yl]methyl]-3-[2-methoxy-5-(trifluoromethyl)phenyl]urea (0.6 g, 39% yield) as a white solid.

Step 3. To a solution of 1-[[2-(2,6-dioxo-3-piperidyl)-4-fluoro-1-oxo-isoindolin-5-yl]methyl]-3-[2-methoxy-5-(trifluoromethyl)phenyl]urea (0.55 g, 1.08 mmol, 1.0 equiv) in DCM (11 mL) was added BBr₃ (2.71 g, 10.82 mmol, 1.04 mL, 10.0 equiv) at −40° C., and then it was stirred at −40° C. for 5 h. The reaction mixture was slowly added to 100 mL sat·NaHCO₃, and then precipitate formed. After filtered, the filter cake was dried. The residue was purified by prep-HPLC (column: Phenomenex luna C18 150*40 mm*15 um; mobile phase: [water(TFA)-ACN]; B %: 20%-50%, 10 min) to afford 1-[[2-(2,6-dioxo-3-piperidyl)-4-fluoro-1-oxo-isoindolin-5-yl]methyl]-3-[2-hydroxy-5-(trifluoromethyl)phenyl]urea (150 mg, 303 umol, 28.05% yield) as a white solid.

### Example 9—Synthesis of Compound I-53: 2-((R)-3-(4-amino-3-(4-phenoxyphenyl)-1H-pyrazolo[3,4-d]pyrimidin-1-yl)piperidine-1-carbonyl)allyl (3-chloro-5-(3-((2-(2,6-dioxopiperidin-3-yl)-1-oxoisoindolin-5-yl)methyl)ureido)benzyl)carbamate

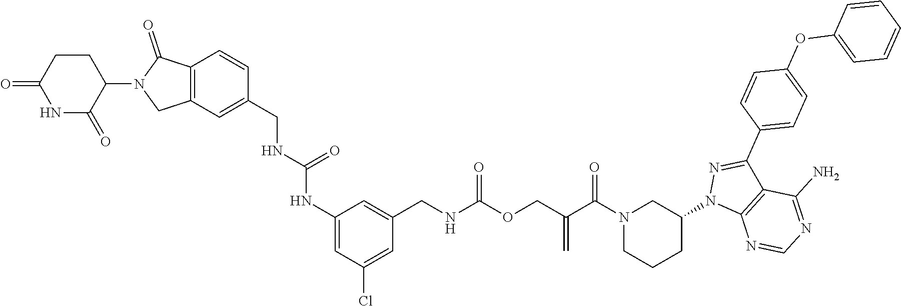 [SMILES: C=C(COC(=O)NCc1cc(Cl)cc(NC(=O)NCc2ccc3c(c2)CN(C2CCC(=O)NC2=O)C3=O)c1)C(=O)N1CCC[C@@H](n2nc(-c3ccc(Oc4ccccc4)cc3)c3c(N)ncnc32)C1]

Compound I-53 was prepared as follows: to a solution of Compound B (53 mg, 94 μmol, 1.0 eq, TFA salt) in THF (1 mL) was added TEA (47 mg, 471 μmol, 65 μL, 5.0 eq) and Compound A (60 mg, 94 μmol, 1.0 eq) in THF (1 mL). The mixture was stirred at 20° C. for 12 h. The reaction mixture was concentrated under reduced pressure to give a residue. The residue was purified by prep-HPLC (column: Phenomenex C18 75×30 mm×3 um; mobile phase: [water(FA)-ACN]; B %: 38%-68%, 7 min) to give compound 2-((R)-3-(4-amino-3-(4-phenoxyphenyl)-1H-pyrazolo3,4-d]pyrimidin-1-yl)piperidine-1-carbonyl)allyl (3-chloro-5-(3-((2-(2,6-dioxopiperidin-3-yl)-1-oxoisoindolin-5-yl)methyl)ureido)benzyl)carbamate (45 mg, 43 μmol, 46% yield, 91% purity) was obtained as an off-white solid. ¹H NMR (400 MHz, DMSO): δ 11.08-10.89 (m, 1H), 8.91 (m, 1H), 8.36-8.13 (m, 1H), 8.11-7.85 (m, 1H), 7.74-7.61 (m, 3H), 7.59-7.54 (m, 1H), 7.49 (s, 1H), 7.47-7.36 (m, 3H), 7.25-7.06 (m, 6H), 6.90-6.75 (m, 2H), 5.53-5.38 (m, 1H), 5.35-5.19 (m, 1H), 5.10 (in, J=5.1, 13.3 Hz, 1H), 4.84-4.63 (m, 2H), 4.57-3.98 (m, 9H), 3.29-3.10 (m, 2H), 2.97-2.77 (m, 2H), 2.34-2.17 (m, 2H), 2.14-2.04 (m, 1H), 2.03-1.93 (m, 1H), 1.91-1.74 (m, 1H), 1.72-1.38 (in, 1H). LC-MS: MS (ES⁺): RT=2.067 min, m/z=952.2 [M+H]M; LC-MS method: AB25

### Example 10—Synthesis of Additional Compounds

The following compounds were prepared using procedures based on that described in Example 9 above.

| | | | RT in | |
|---|---|---|---|---|
| | | | LC-MS | LC-MS |
| No. | Chemical Structure | MH+ | (min) | Method |
| II-184 | 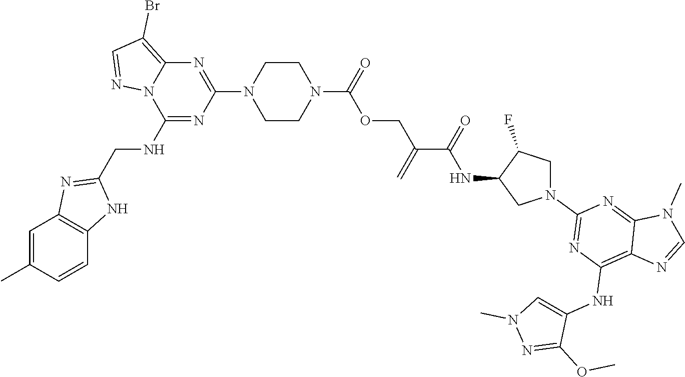 [SMILES: C=C(COC(=O)N1CCN(c2nc(NCc3nc4cc(C)ccc4[nH]3)n3ncc(Br)c3n2)CC1)C(=O)N[C@@H]1CN(c2nc(Nc3cn(C)nc3OC)c3ncn(C)c3n2)C[C@H]1F] | 457.9, 915.2 | 2.217 | AB10 |
| II-185 | 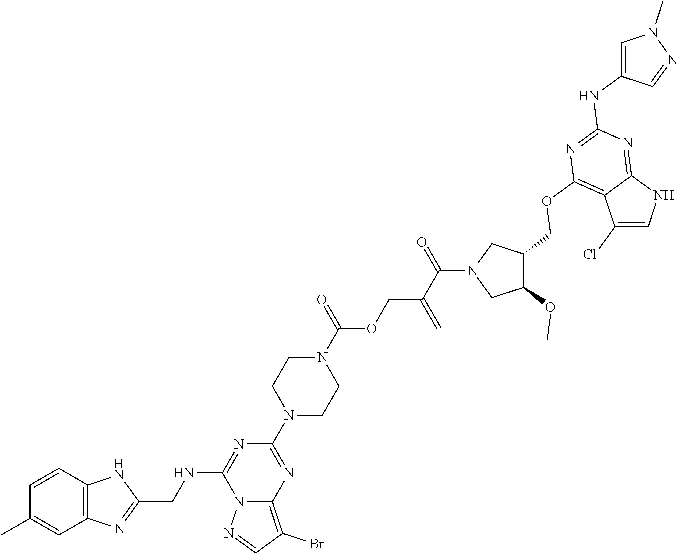 [SMILES: C=C(COC(=O)N1CCN(c2nc(NCc3nc4cc(C)ccc4[nH]3)n3ncc(Br)c3n2)CC1)C(=O)N1C[C@H](COc2nc(Nc3cnn(C)c3)nc3[nH]cc(Cl)c23)[C@@H](OC)C1] | 466.1, 931.2 | 1.844 | AB25 |
| V-4 | 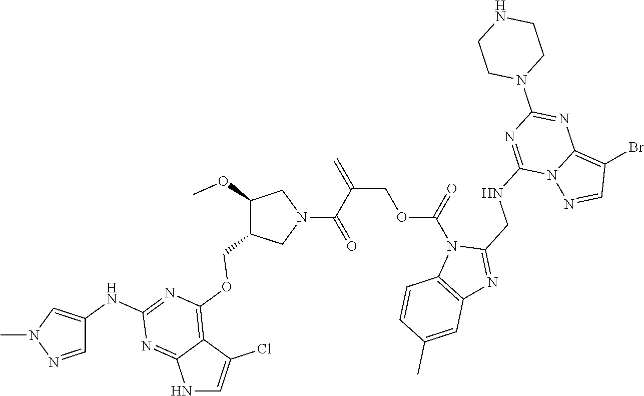 [SMILES: C=C(COC(=O)n1c(CNc2nc(N3CCNCC3)nc3c(Br)cnn23)nc2cc(C)ccc21)C(=O)N1C[C@H](COc2nc(Nc3cnn(C)c3)nc3[nH]cc(Cl)c23)[C@@H](OC)C1] | 466.1, 931.2 | 1.93 | AB25 |
| II-187 | 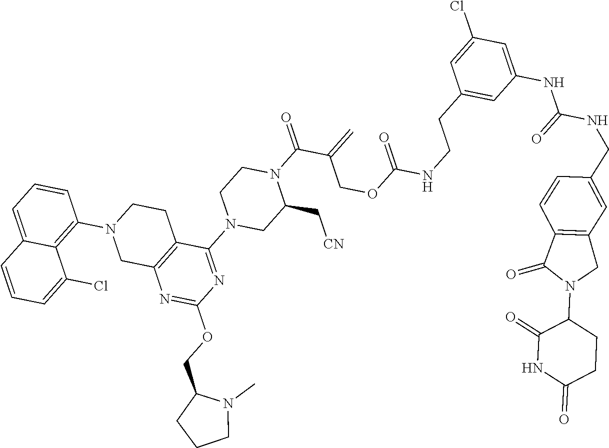 [SMILES: C=C(COC(=O)NCCc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] | 556.3, 1111.4 | 1.987 | AB25 |
| I-65 | 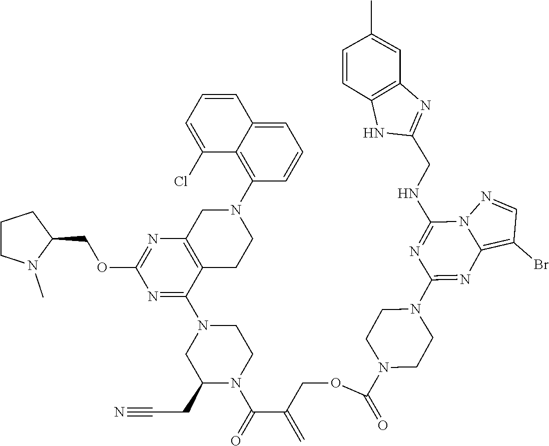 [SMILES: C=C(COC(=O)N1CCN(c2nc(NCc3nc4cc(C)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] | 543.1, 1085.2 | 2.375 | AB10 |
| I-73 | 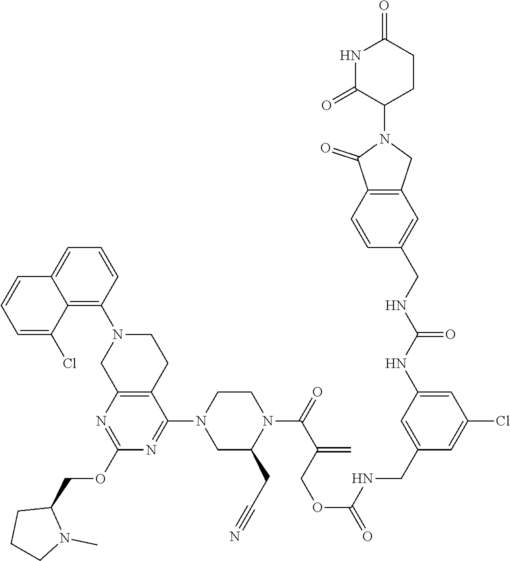 [SMILES: C=C(COC(=O)NCc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] | 549.2, 1097.4 | 1.939 | AB25 |
| I-74 | 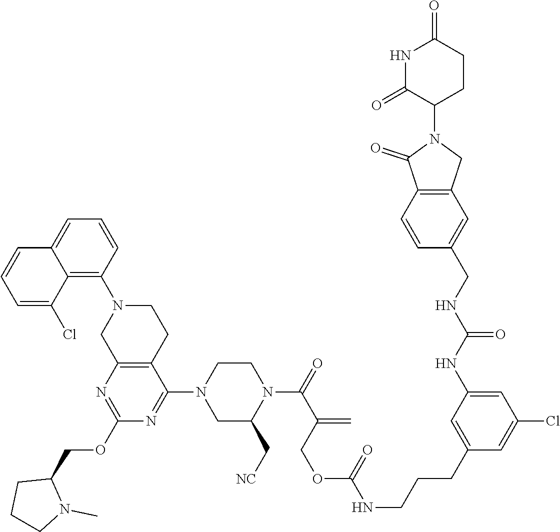 [SMILES: C=C(COC(=O)NCCCc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] | 564.2, 1127.4 | 2.023 | AB25 |
| II-189 | 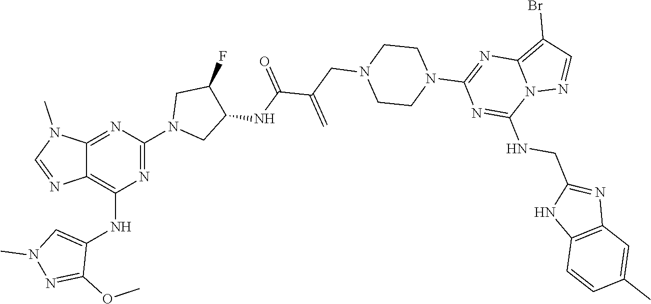 | 888 | 1.528 | AB25 |

### Example 11—Synthesis of Compound I-72: 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carbonyl]allyl 2-[[2-(2,6-dioxo-3-piperidyl)-1-oxo-isoindolin-5-yl]methylcarbamoylamino]-4-methyl-thiophene-3-carboxylate

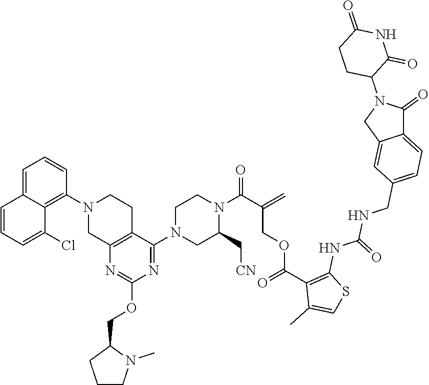 [SMILES: C=C(COC(=O)c1c(C)csc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N]

To a mixture of Compound J (22 mg, 49 μmol, 1.0 eq) and Compound K (30 mg, 49 μmol, 1.0 eq) in DMF (3 mL) was added DIEA (31 mg, 243 μmol, 42 μL, 5 eq) and DCC (30 mg, 146 μmol, 30 μL, 3 eq). The mixture was stirred at 20° C. for 12 h. 0.1 mL water was added to quench the reaction. The reaction mixture was purified by prep-HPLC (column: Phenomenex luna C18 150×25 mm×10 um; mobile phase: [water(FA)-ACN]; B %: 31%-61%, 10 min) to give the desired product 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carbonyl]allyl 2-[[2-(2,6-dioxo-3-piperidyl)-1-oxo-isoindolin-5-yl]methylcarbamoylamino]-4-methyl-thiophene-3-carboxylate (10.93 mg, 10.23 μmol, 21% yield, 98.7% purity) as a white solid. LC-MS: MS (ES⁺): RT=2.655 min, m/z=1054.7 [M+H⁺]; LC-MS Method: AB05. ¹H NMR: (400 MHz, CD₃OD) δ=7.84-7.82 (d, J=8.0 Hz, 1H), 7.75 (m, 1H), 7.60 (m, 1H), 7.55-7.48 (m, 4H), 7.09 (m, 1H), 7.40 (m, 1H), 7.38 (m, 1H), 6.37 (s, 1H), 5.78 (s, 1H), 5.60 (s, 1H), 5.09-5.06 (m, 3H), 4.53-4.47 (m, 6H), 4.29 (m, 2H), 4.00 (m, 2H), 3.70 (m, 1H), 3.60 (m, 1H), 3.35 (m, 1H), 3.15-2.80 (m, 7H), 2.80-2.50 (m, 8H), 2.50-2.34 (m, 4H), 2.30-1.81 (m, 6H).

### Example 12—Synthesis of Additional Compounds

The following compounds were prepared based on procedures described for Example 9.

| Compound | | Starting |
|---|---|---|
| No. | Chemical Structure | Material |
| I-58 | 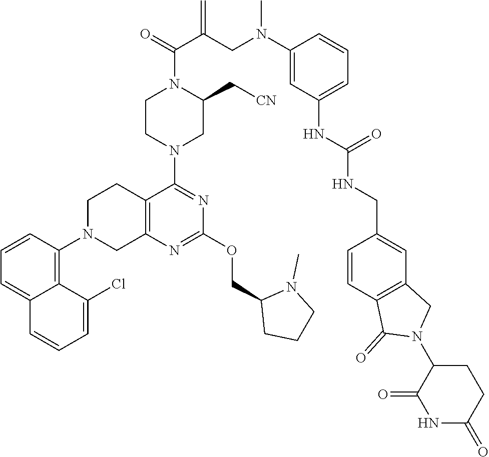 [SMILES: C=C(CN(C)c1cccc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] | O, K |
| II-182 | 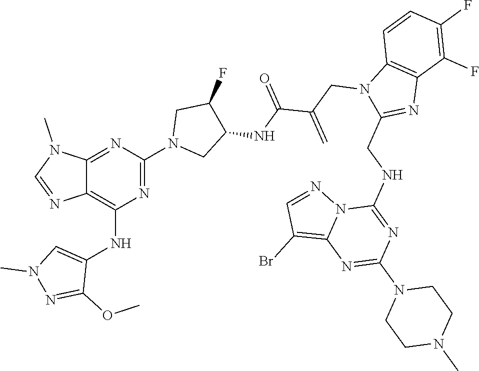 [SMILES: C=C(Cn1c(CNc2nc(N3CCN(C)CC3)nc3c(Br)cnn23)nc2c(F)c(F)ccc21)C(=O)N[C@@H]1CN(c2nc(Nc3cn(C)nc3OC)c3ncn(C)c3n2)C[C@H]1F] | P, Q |
| II-181 | 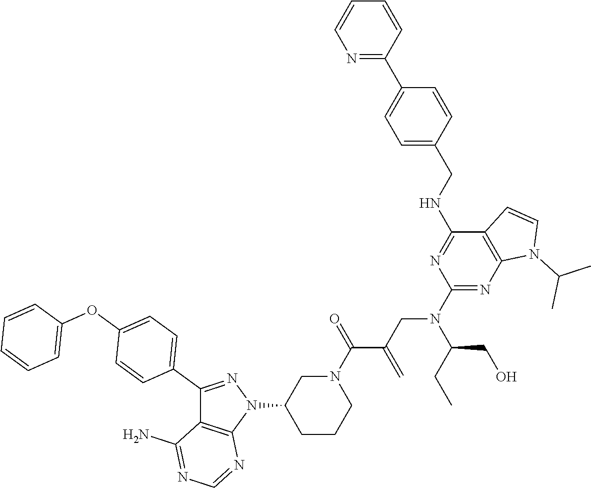 [SMILES: C=C(CN(c1nc(NCc2ccc(-c3ccccn3)cc2)c2ccn(C(C)C)c2n1)[C@H](CC)CO)C(=O)N1CCC[C@H](n2nc(-c3ccc(Oc4ccccc4)cc3)c3c(N)ncnc32)C1] | R, N |
| I-30 | 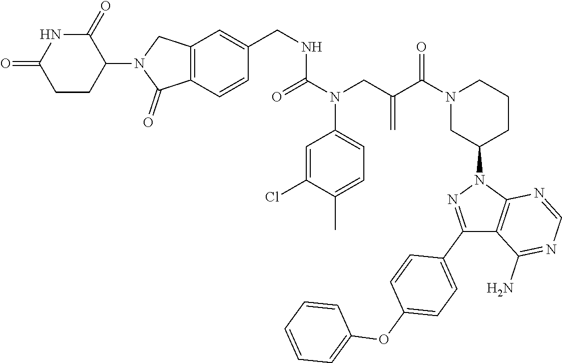 [SMILES: C=C(CN(C(=O)NCc1ccc2c(c1)CN(C1CCC(=O)NC1=O)C2=O)c1ccc(C)c(Cl)c1)C(=O)N1CCC[C@@H](n2nc(-c3ccc(Oc4ccccc4)cc3)c3c(N)ncnc32)C1] | S, N |
| I-34 | 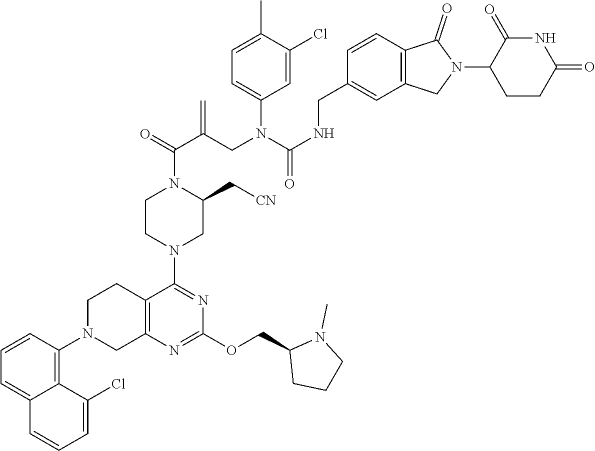 [SMILES: C=C(CN(C(=O)NCc1ccc2c(c1)CN(C1CCC(=O)NC1=O)C2=O)c1ccc(C)c(Cl)c1)C(=O)N1CCN(c2nc(OC[C@@H]3CCCN3C)nc3c2CCN(c2cccc4cccc(Cl)c24)C3)C[C@@H]1CC#N] | S, K |
| I-37 | 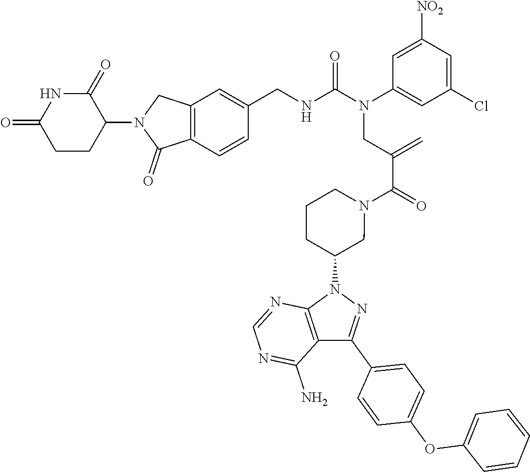 [SMILES: C=C(CN(C(=O)NCc1ccc2c(c1)CN(C1CCC(=O)NC1=O)C2=O)c1cc(Cl)cc([N+](=O)[O-])c1)C(=O)N1CCC[C@@H](n2nc(-c3ccc(Oc4ccccc4)cc3)c3c(N)ncnc32)C1] | T, N |
| I-38 | 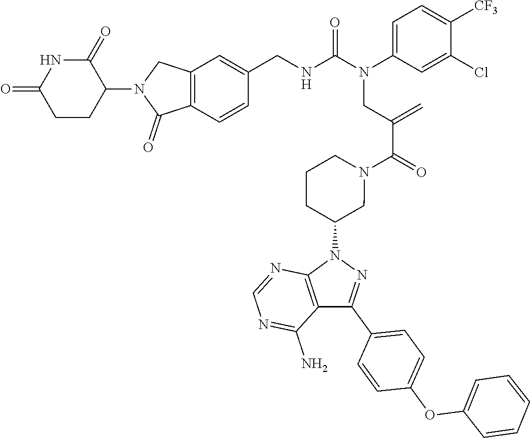 [SMILES: C=C(CN(C(=O)NCc1ccc2c(c1)CN(C1CCC(=O)NC1=O)C2=O)c1ccc(C(F)(F)F)c(Cl)c1)C(=O)N1CCC[C@@H](n2nc(-c3ccc(Oc4ccccc4)cc3)c3c(N)ncnc32)C1] | U, N |
| I-39 | 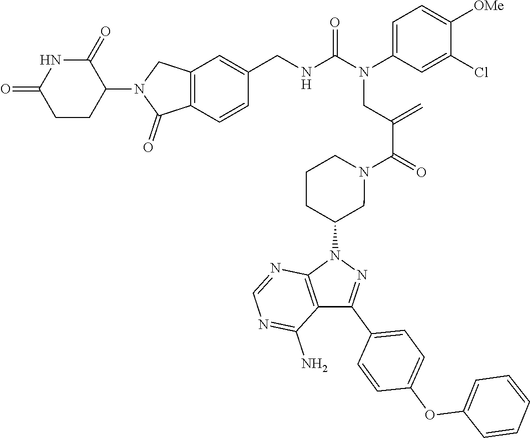 [SMILES: C=C(CN(C(=O)NCc1ccc2c(c1)CN(C1CCC(=O)NC1=O)C2=O)c1ccc(OC)c(Cl)c1)C(=O)N1CCC[C@@H](n2nc(-c3ccc(Oc4ccccc4)cc3)c3c(N)ncnc32)C1] | V, N |
| I-40 | 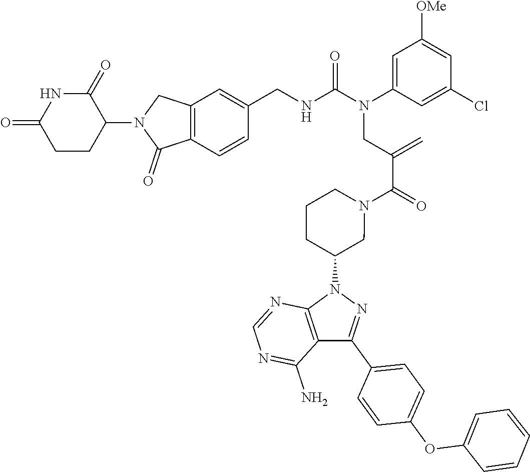 [SMILES: C=C(CN(C(=O)NCc1ccc2c(c1)CN(C1CCC(=O)NC1=O)C2=O)c1cc(Cl)cc(OC)c1)C(=O)N1CCC[C@@H](n2nc(-c3ccc(Oc4ccccc4)cc3)c3c(N)ncnc32)C1] | W, N |
| I-41 | 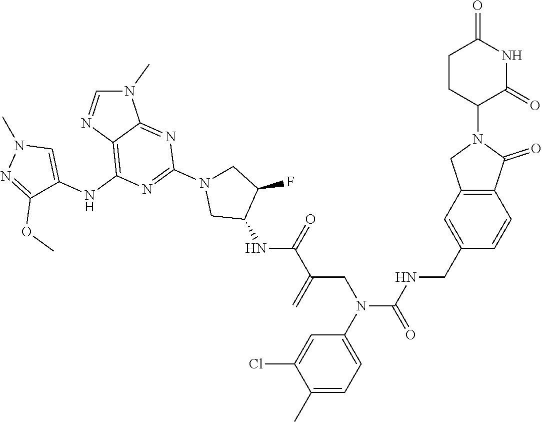 [SMILES: C=C(CN(C(=O)NCc1ccc2c(c1)CN(C1CCC(=O)NC1=O)C2=O)c1ccc(C)c(Cl)c1)C(=O)N[C@@H]1CN(c2nc(Nc3cn(C)nc3OC)c3ncn(C)c3n2)C[C@H]1F] | X, Q |
| I-42 | 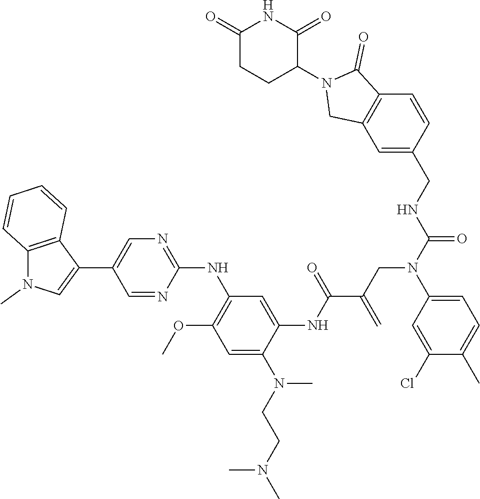 | X, Y |
| I-70 | 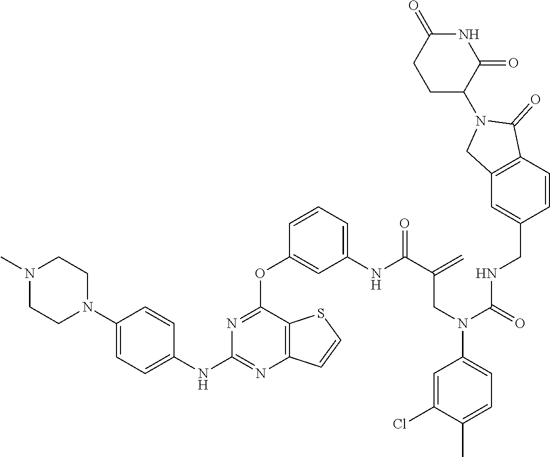 [SMILES: C=C(CN(C(=O)NCc1ccc2c(c1)CN(C1CCC(=O)NC1=O)C2=O)c1ccc(C)c(Cl)c1)C(=O)Nc1cccc(Oc2nc(Nc3ccc(N4CCN(C)CC4)cc3)nc3ccsc23)c1] | X, Z |
| I-71 | 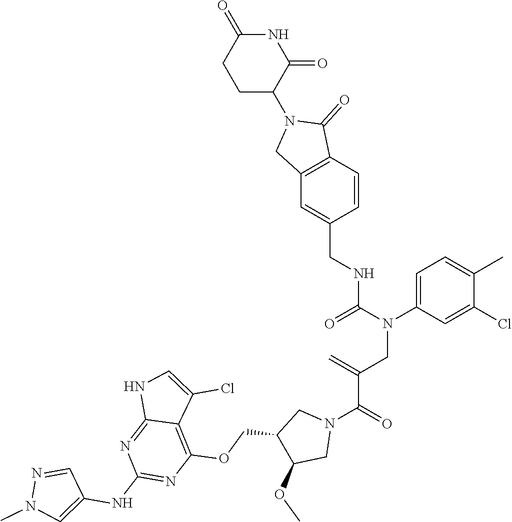 [SMILES: C=C(CN(C(=O)NCc1ccc2c(c1)CN(C1CCC(=O)NC1=O)C2=O)c1ccc(C)c(Cl)c1)C(=O)N1C[C@@H](COc2nc(Nc3cnn(C)c3)nc3[nH]cc(Cl)c23)[C@H](OC)C1] | X, AA |
| I-31 | 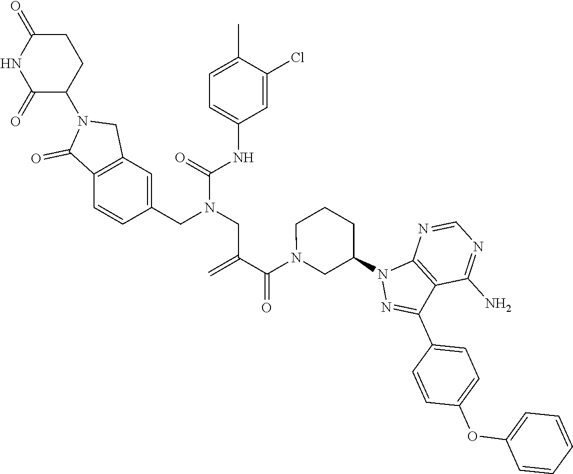 [SMILES: C=C(CN(Cc1ccc2c(c1)CN(C1CCC(=O)NC1=O)C2=O)C(=O)Nc1ccc(C)c(Cl)c1)C(=O)N1CCC[C@@H](n2nc(-c3ccc(Oc4ccccc4)cc3)c3c(N)ncnc32)C1] | AB, N |
| I-33 | 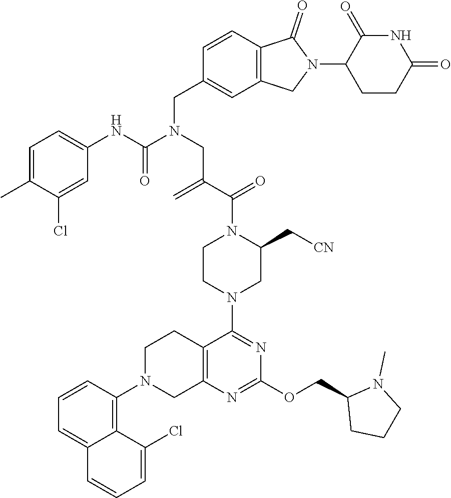 [SMILES: C=C(CN(Cc1ccc2c(c1)CN(C1CCC(=O)NC1=O)C2=O)C(=O)Nc1ccc(C)c(Cl)c1)C(=O)N1CCN(c2nc(OC[C@@H]3CCCN3C)nc3c2CCN(c2cccc4cccc(Cl)c24)C3)C[C@@H]1CC#N] | AB, K |
| I-32 | 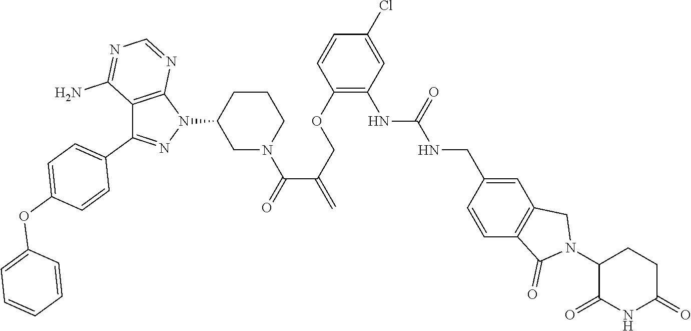 [SMILES: C=C(COc1ccc(Cl)cc1NC(=O)NCc1ccc2c(c1)CN(C1CCC(=O)NC1=O)C2=O)C(=O)N1CCC[C@@H](n2nc(-c3ccc(Oc4ccccc4)cc3)c3c(N)ncnc32)C1] | AC, N |
| I-32 | 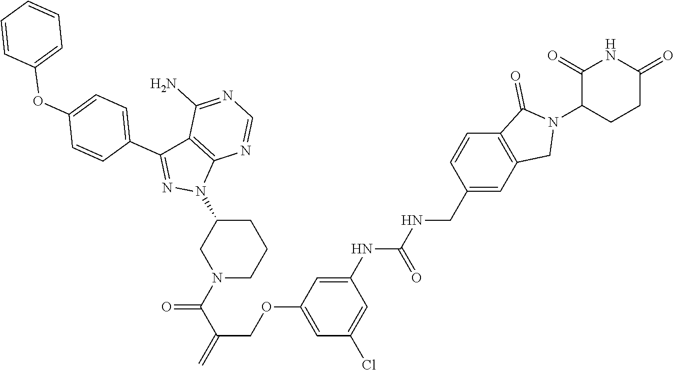 [SMILES: C=C(COc1cc(Cl)cc(NC(=O)NCc2ccc3c(c2)CN(C2CCC(=O)NC2=O)C3=O)c1)C(=O)N1CCC[C@@H](n2nc(-c3ccc(Oc4ccccc4)cc3)c3c(N)ncnc32)C1] | AD, N |
| I-35 | 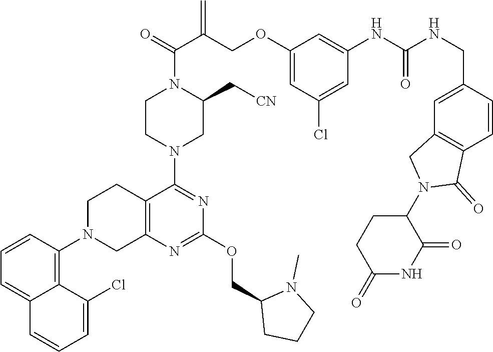 [SMILES: C=C(COc1cc(Cl)cc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] | AD, K |
| I-36 | 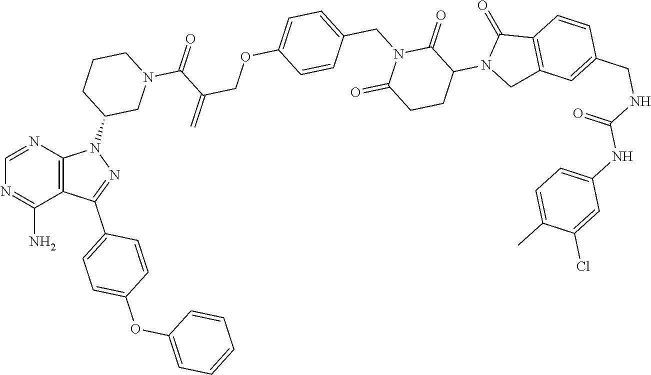 [SMILES: C=C(COc1ccc(CN2C(=O)CCC(N3Cc4cc(CNC(=O)Nc5ccc(C)c(Cl)c5)ccc4C3=O)C2=O)cc1)C(=O)N1CCC[C@@H](n2nc(-c3ccc(Oc4ccccc4)cc3)c3c(N)ncnc32)C1] | AE, K |
| I-43 | 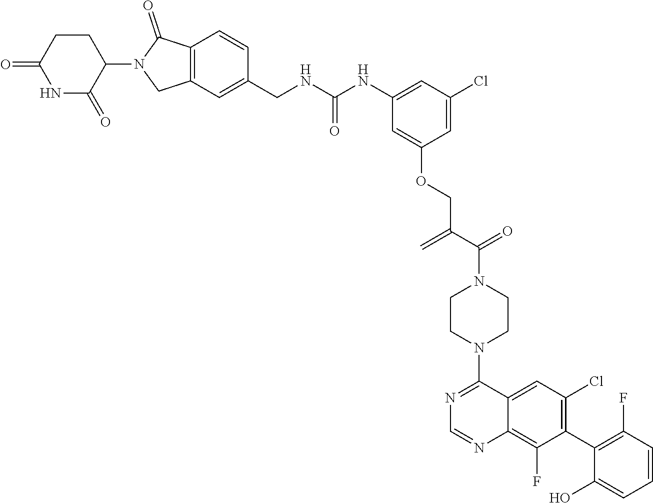 [SMILES: C=C(COc1cc(Cl)cc(NC(=O)NCc2ccc3c(c2)CN(C2CCC(=O)NC2=O)C3=O)c1)C(=O)N1CCN(c2ncnc3c(F)c(-c4c(O)cccc4F)c(Cl)cc23)CC1] | AD, AF |
| I-44 | 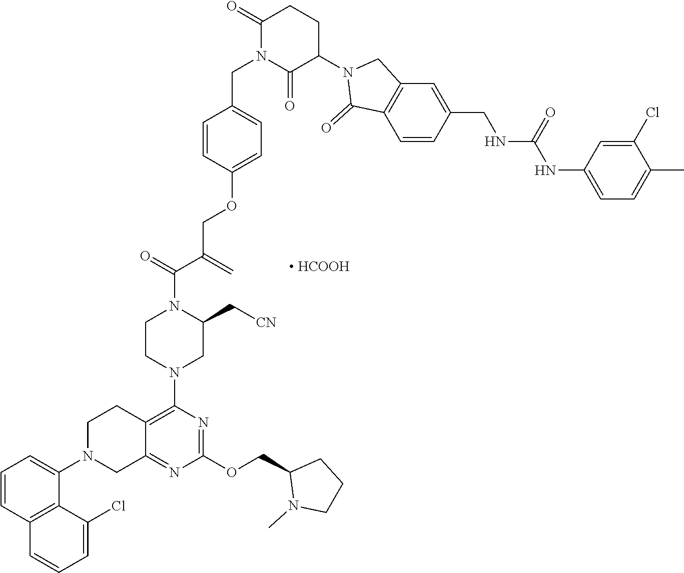 [SMILES: C=C(COc1ccc(CN2C(=O)CCC(N3Cc4cc(CNC(=O)Nc5ccc(C)c(Cl)c5)ccc4C3=O)C2=O)cc1)C(=O)N1CCN(c2nc(OC[C@H]3CCCN3C)nc3c2CCN(c2cccc4cccc(Cl)c24)C3)C[C@@H]1CC#N ; "· HCOOH"] | AE, K |
| I-45 | 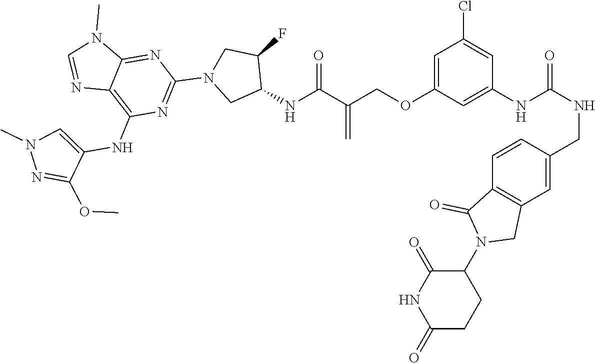 [SMILES: C=C(COc1cc(Cl)cc(NC(=O)NCc2ccc3c(c2)CN(C2CCC(=O)NC2=O)C3=O)c1)C(=O)N[C@@H]1CN(c2nc(Nc3cn(C)nc3OC)c3ncn(C)c3n2)C[C@H]1F] | AD, Q |
| I-46 | 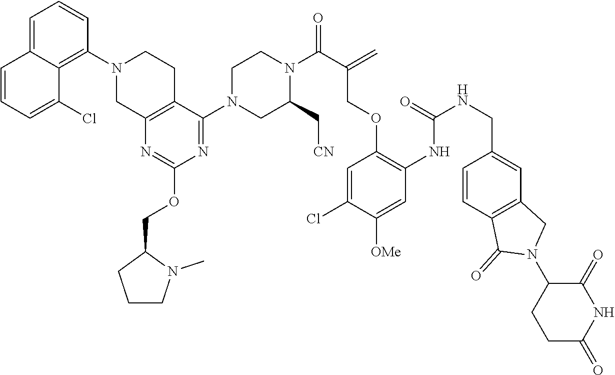 [SMILES: C=C(COc1cc(Cl)c(OC)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] | AG, K |
| I-47 | 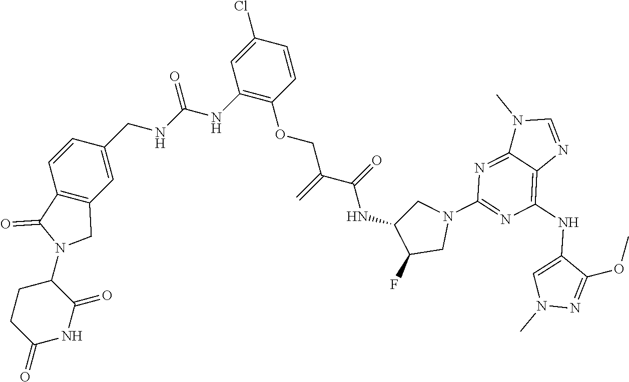 [SMILES: C=C(COc1ccc(Cl)cc1NC(=O)NCc1ccc2c(c1)CN(C1CCC(=O)NC1=O)C2=O)C(=O)N[C@@H]1CN(c2nc(Nc3cn(C)nc3OC)c3ncn(C)c3n2)C[C@H]1F] | AC, Q |
| I-48 | 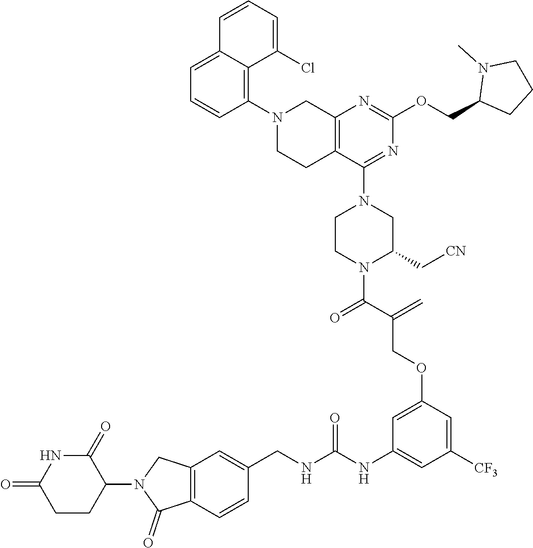 [SMILES: C=C(COc1cc(NC(=O)NCc2ccc3c(c2)CN(C2CCC(=O)NC2=O)C3=O)cc(C(F)(F)F)c1)C(=O)N1CCN(c2nc(OC[C@@H]3CCCN3C)nc3c2CCN(c2cccc4cccc(Cl)c24)C3)C[C@@H]1CC#N] | AH, K |
| I-49 | 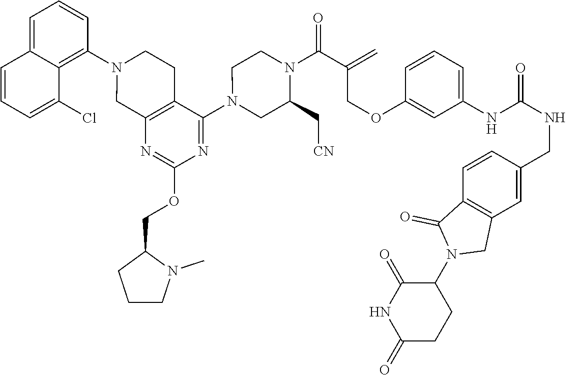 [SMILES: C=C(COc1cccc(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] | AI, K |
| I-50 | 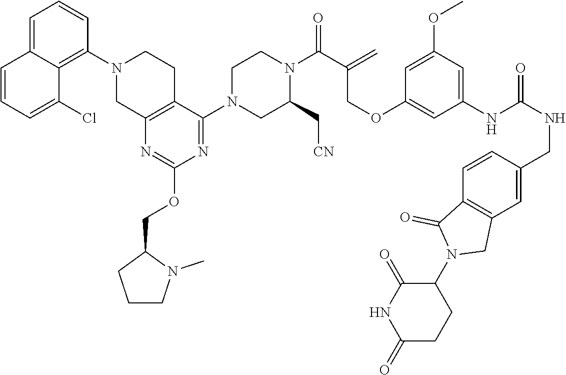 [SMILES: C=C(COc1cc(NC(=O)NCc2ccc3c(c2)CN(C2CCC(=O)NC2=O)C3=O)cc(OC)c1)C(=O)N1CCN(c2nc(OC[C@@H]3CCCN3C)nc3c2CCN(c2cccc4cccc(Cl)c24)C3)C[C@@H]1CC#N] | AJ, K |
| I-52 | 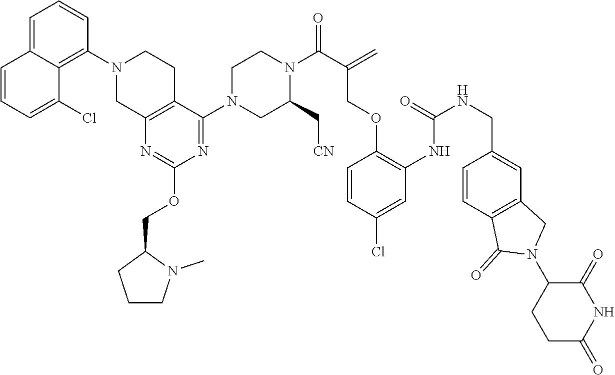 [SMILES: C=C(COc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] | AC, K |
| I-54 | 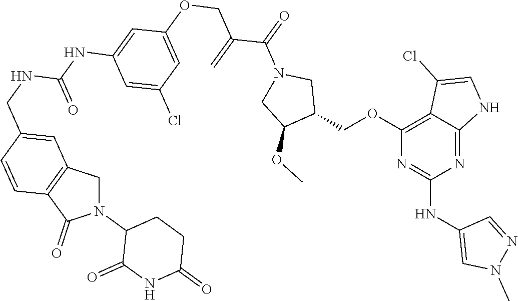 [SMILES: C=C(COc1cc(Cl)cc(NC(=O)NCc2ccc3c(c2)CN(C2CCC(=O)NC2=O)C3=O)c1)C(=O)N1C[C@H](COc2nc(Nc3cnn(C)c3)nc3[nH]cc(Cl)c23)[C@@H](OC)C1] | AD, AA |
| I-55 | 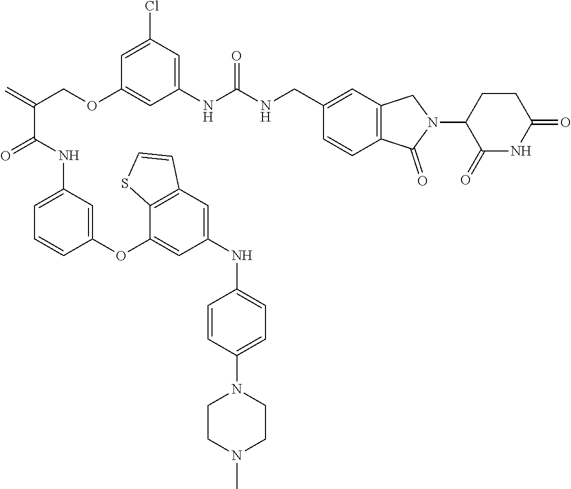 [SMILES: C=C(COc1cc(Cl)cc(NC(=O)NCc2ccc3c(c2)CN(C2CCC(=O)NC2=O)C3=O)c1)C(=O)Nc1cccc(Oc2cc(Nc3ccc(N4CCN(C)CC4)cc3)cc3ccsc23)c1] | AD, Z |
| I-56 | 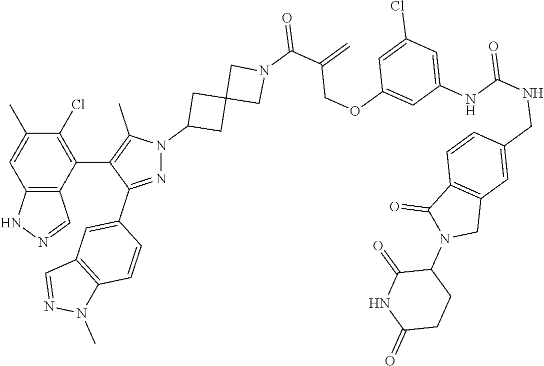 [SMILES: C=C(COc1cc(Cl)cc(NC(=O)NCc2ccc3c(c2)CN(C2CCC(=O)NC2=O)C3=O)c1)C(=O)N1CC2(CC(n3nc(-c4ccc5c(cnn5C)c4)c(-c4c(Cl)c(C)cc5[nH]ncc45)c3C)C2)C1] | AD, AK |
| I-57 | 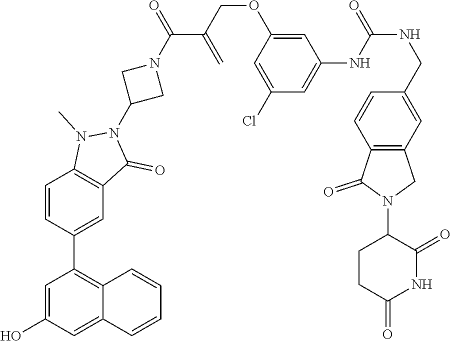 [SMILES: C=C(COc1cc(Cl)cc(NC(=O)NCc2ccc3c(c2)CN(C2CCC(=O)NC2=O)C3=O)c1)C(=O)N1CC(n2c(=O)c3cc(-c4cc(O)cc5ccccc45)ccc3n2C)C1] | AD, AL |
| II-183 | 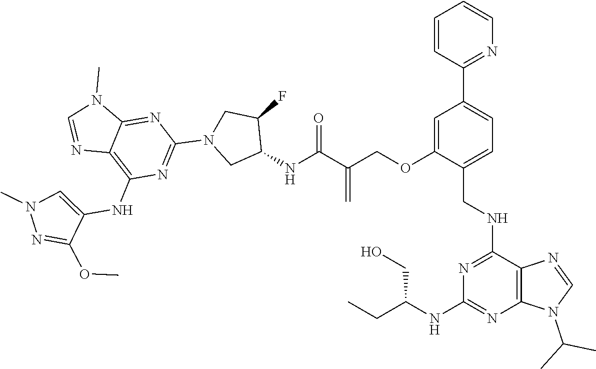 [SMILES: C=C(COc1cc(-c2ccccn2)ccc1CNc1nc(N[C@H](CC)CO)nc2c1ncn2C(C)C)C(=O)N[C@@H]1CN(c2nc(Nc3cn(C)nc3OC)c3ncn(C)c3n2)C[C@H]1F] | R, Q |
| I-59 | 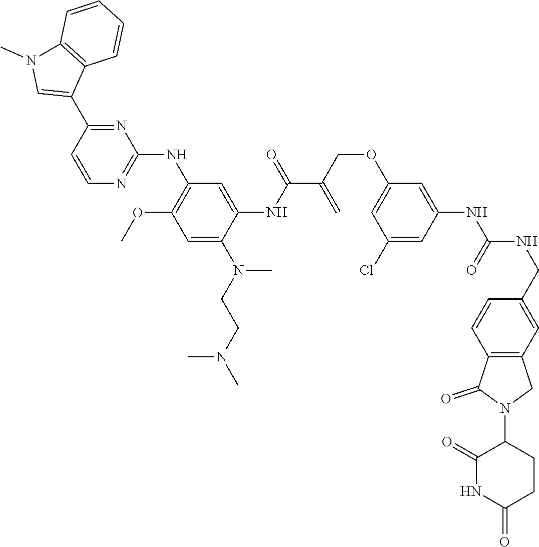 [SMILES: C=C(COc1cc(Cl)cc(NC(=O)NCc2ccc3c(c2)CN(C2CCC(=O)NC2=O)C3=O)c1)C(=O)Nc1cc(Nc2nccc(-c3cn(C)c4ccccc34)n2)c(OC)cc1N(C)CCN(C)C] | AD, Y |
| I-61 | 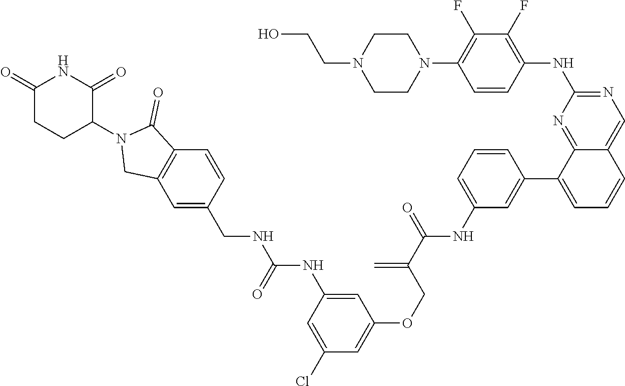 [SMILES: C=C(COc1cc(Cl)cc(NC(=O)NCc2ccc3c(c2)CN(C2CCC(=O)NC2=O)C3=O)c1)C(=O)Nc1cccc(-c2cccc3cnc(Nc4ccc(N5CCN(CCO)CC5)c(F)c4F)nc23)c1] | AD, AM |
| II-186 | 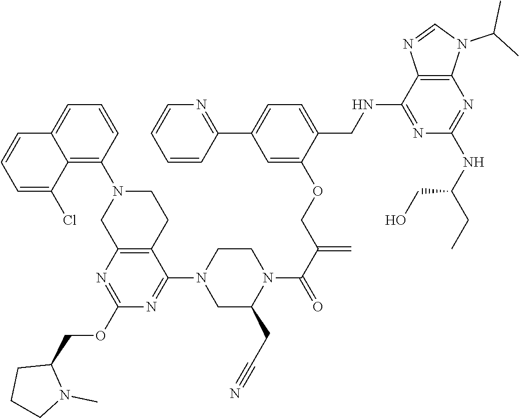 [SMILES: C=C(COc1cc(-c2ccccn2)ccc1CNc1nc(N[C@H](CC)CO)nc2c1ncn2C(C)C)C(=O)N1CCN(c2nc(OC[C@@H]3CCCN3C)nc3c2CCN(c2cccc4cccc(Cl)c24)C3)C[C@@H]1CC#N] | R, K |
| I-64 | 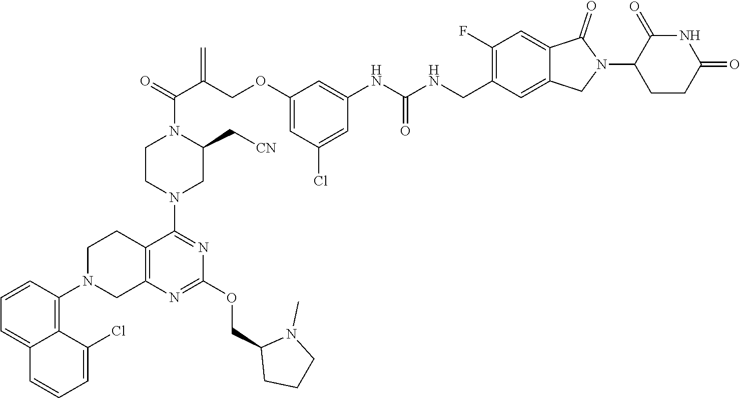 [SMILES: C=C(COc1cc(Cl)cc(NC(=O)NCc2cc3c(cc2F)C(=O)N(C2CCC(=O)NC2=O)C3)c1)C(=O)N1CCN(c2nc(OC[C@@H]3CCCN3C)nc3c2CCN(c2cccc4cccc(Cl)c24)C3)C[C@@H]1CC#N] | AN, K |
| I-66 | 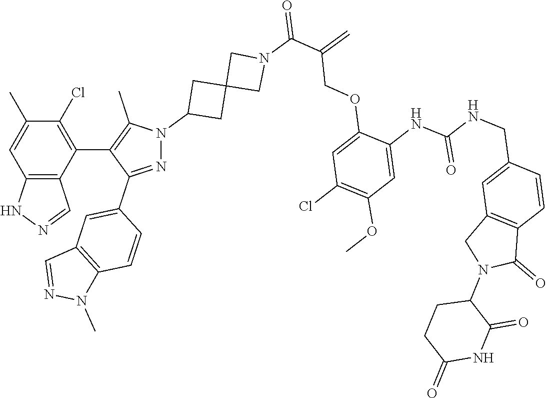 [SMILES: C=C(COc1cc(Cl)c(OC)cc1NC(=O)NCc1ccc2c(c1)CN(C1CCC(=O)NC1=O)C2=O)C(=O)N1CC2(CC(n3nc(-c4ccc5c(cnn5C)c4)c(-c4c(Cl)c(C)cc5[nH]ncc45)c3C)C2)C1] | AG, AK |
| I-67 | 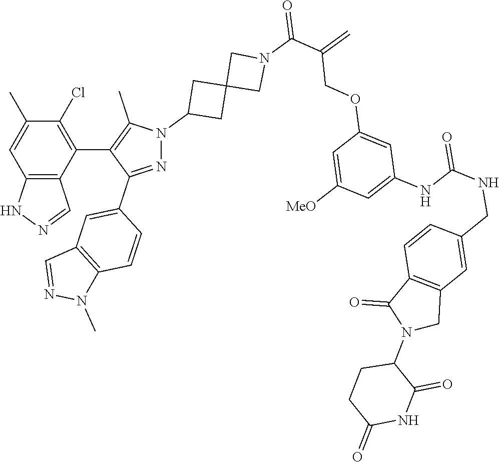 [SMILES: C=C(COc1cc(NC(=O)NCc2ccc3c(c2)CN(C2CCC(=O)NC2=O)C3=O)cc(OC)c1)C(=O)N1CC2(CC(n3nc(-c4ccc5c(cnn5C)c4)c(-c4c(Cl)c(C)cc5[nH]ncc45)c3C)C2)C1] | AJ, AK |
| I-68 | 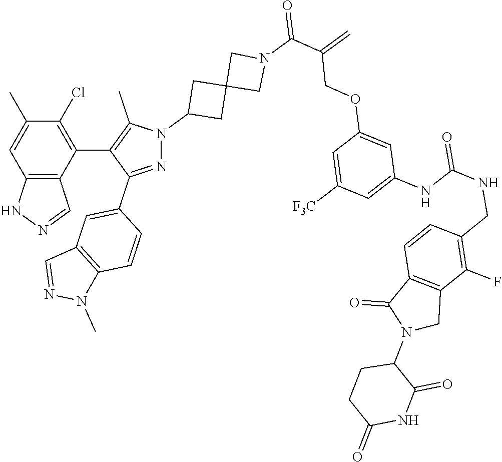 [SMILES: C=C(COc1cc(NC(=O)NCc2ccc3c(c2F)CN(C2CCC(=O)NC2=O)C3=O)cc(C(F)(F)F)c1)C(=O)N1CC2(CC(n3nc(-c4ccc5c(cnn5C)c4)c(-c4c(Cl)c(C)cc5[nH]ncc45)c3C)C2)C1] | AO, AK |
| I-69 | 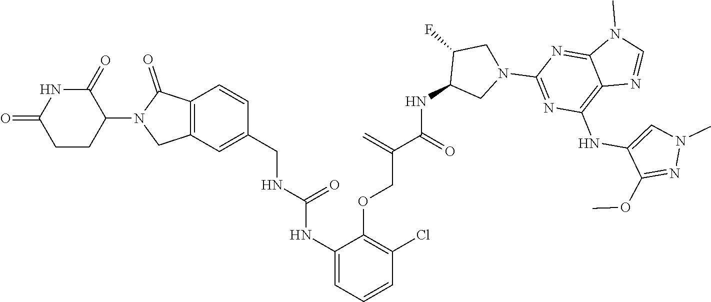 [SMILES: C=C(COc1c(Cl)cccc1NC(=O)NCc1ccc2c(c1)CN(C1CCC(=O)NC1=O)C2=O)C(=O)N[C@@H]1CN(c2nc(Nc3cn(C)nc3OC)c3ncn(C)c3n2)C[C@H]1F] | AP, Q |
| I-76 | 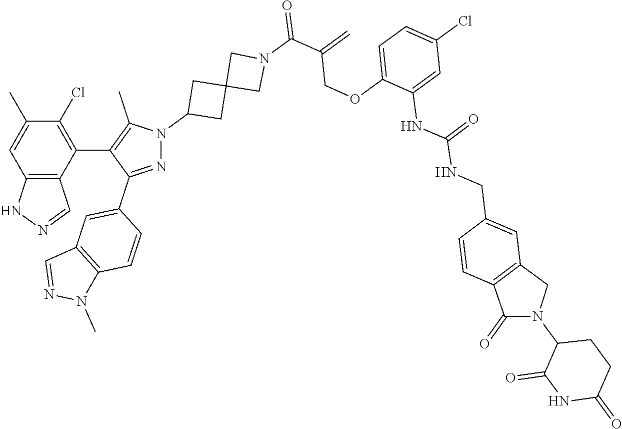 [SMILES: C=C(COc1ccc(Cl)cc1NC(=O)NCc1ccc2c(c1)CN(C1CCC(=O)NC1=O)C2=O)C(=O)N1CC2(CC(n3nc(-c4ccc5c(cnn5C)c4)c(-c4c(Cl)c(C)cc5[nH]ncc45)c3C)C2)C1] | AC, AK |
| I-77 | 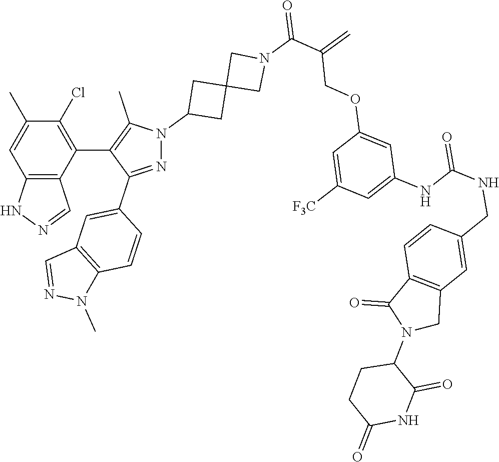 [SMILES: C=C(COc1cc(NC(=O)NCc2ccc3c(c2)CN(C2CCC(=O)NC2=O)C3=O)cc(C(F)(F)F)c1)C(=O)N1CC2(CC(n3nc(-c4ccc5c(cnn5C)c4)c(-c4c(Cl)c(C)cc5[nH]ncc45)c3C)C2)C1] | AH, AK |

Mass spectroscopic data and retention time in the LC-MS for the above compounds in provided in the following table.

| Compound | | RT in the | LC-MS |
|---|---|---|---|
| No. | MH+ | LC-MS (min) | Method |
| I-58 | 510.2, 1019.4 | 1.897 | AB25 |
| II-182 | 905.6 | 2.273 | AB10 |
| II-181 | 442.7, 884.4 | 2.009 | AB25 |
| I-30 | 447.0, 893.2 | 2.186 | AB05 |
| I-34 | 520 | 2.683 | AB05 |
| I-37 | 924.5 | 2.64 | AB05 |
| I-38 | 947.5 | 2.737 | AB05 |
| I-39 | 909.5 | 2.645 | AB05 |
| I-40 | 909.6 | 2.62 | AB05 |
| I-41 | 868.2, 434.7 | 2.607 | AB01 |
| I-42 | 476.7, 952.3 | 1.744 | AB25 |
| I-70 | 939.5 | 2.603 | AB05 |
| I-71 | 884.5 | 2.48 | AB05 |
| I-31 | 893.2 | 2.782 | AB05 |
| I-33 | 520 | 2.64 | AB05 |
| I-32 | 895.1 | 2.133 | AB25 |
| I-32 | 895.2 | 2.163 | AB25 |
| I-35 | 521.7, 1040.3 | 2.028 | AB25 |
| I-36 | 999.5 | 1.79 | Method B |
| I-43 | 443 | 1.916 | AB10 |
| I-44 | 1144.4 | 1.40 | Method B |
| I-45 | 870.2 | 2.553 | AB01 |
| I-46 | 536.7, 1070.3 | 2.06 | AB25 |
| I-47 | 435.7, 870.2 | 2.568 | AB01 |
| I-48 | 537.7, 1074.3 | 2.09 | AB25 |
| I-49 | 503.7, 1006.3 | 1.889 | AB25 |
| I-50 | 518.7, 1036.3 | 1.913 | AB25 |
| I-52 | 521.2, 1042.2 | 2.045 | AB25 |
| I-54 | 444.6, 866.1 | 2.409 | AB10 |
| I-55 | 471.2, 941.1 | 1.939 | AB25 |
| I-56 | 490.7, 980.0 | 2.736 | AB10 |
| I-57 | 854.2 | 2.143 | AB25 |
| II-183 | 438.2, 875.3 | 2.096 | AB10 |
| I-59 | 477.6, 954.3 | 1.708 | AB25 |
| I-61 | 493.2, 985.2 | 1.994 | AB25 |
| II-186 | 523.7, 1045.4 | 0.701 | AB40 |
| I-64 | 1058.6 | 2.605 | AB10 |
| I-66 | 1012.3 | 2.783 | AB10 |
| I-67 | 488.6, 976.3 | 2.628 | AB10 |
| I-68 | 516.7, 1032.1 | 2.494 | AB25 |
| I-69 | 870.2 | 2.662 | AB01 |
| I-76 | 490.6, 980.3 | 2.773 | AB10 |
| I-77 | 507.8, 1014.3 | 2.446 | AB25 |

### Example 12—Synthesis of Compound 1-[3-chloro-5-[2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carbonyl]allyloxy]phenyl]-3-[[2-(2,6-dioxo-3-piperidyl)-4-fluoro-1-oxo-isoindolin-5-yl]methyl]urea

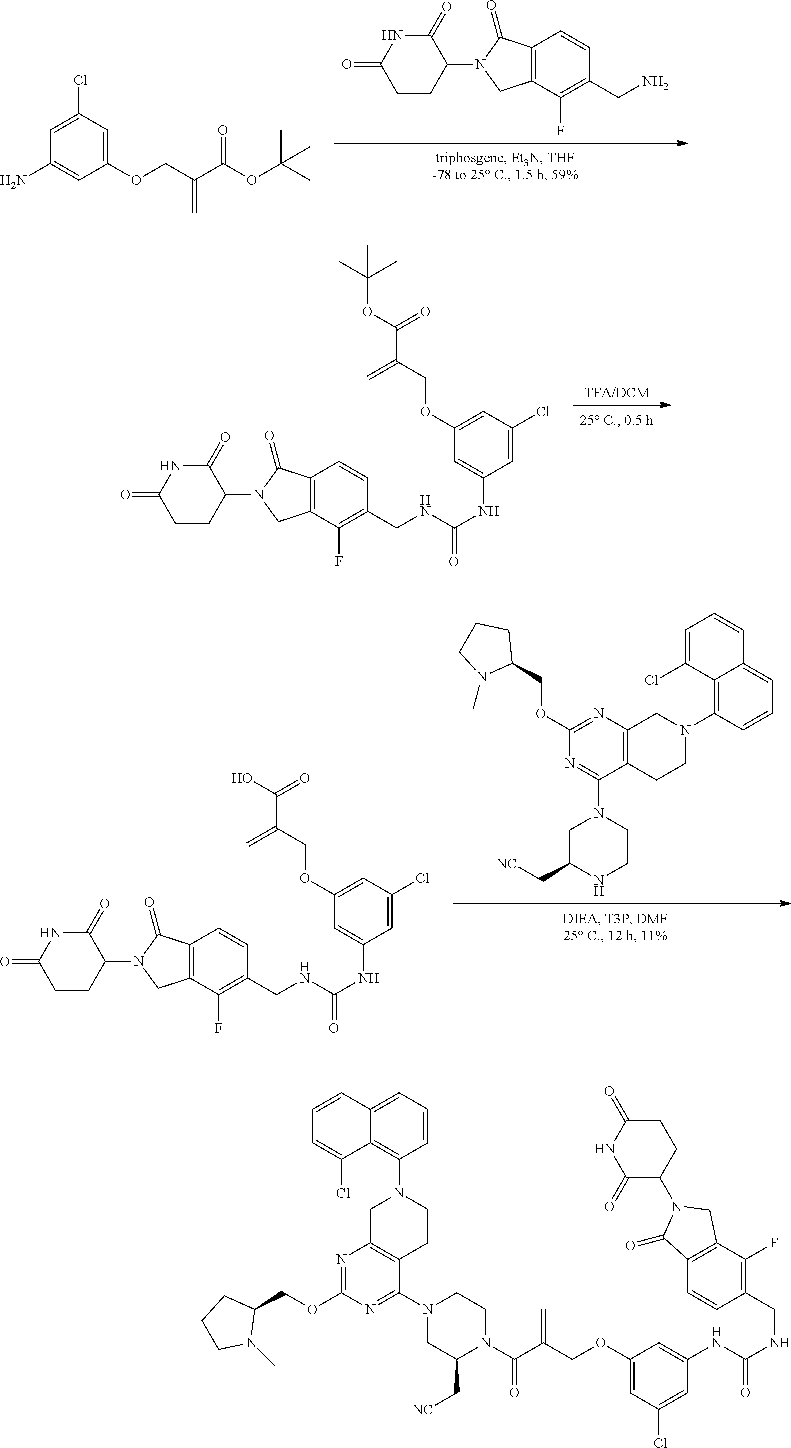

Step 1. To a solution of triphosgene (91.7 mg, 3.09 mmol, 0.55 eq) in THF (5 mL) was added Et₃N (285 mg, 2.82 mmol, 392 μL, 5.0 eq) and tert-butyl 2-[(3-amino-5-chloro-phenoxy) methyl]prop-2-enoate (160 mg, 564 μmol, 1.0 eq). The mixture was stirred at −78° C. for 0.5 h. Then 3-[5-(aminomethyl)-4-fluoro-1-oxo-isoindolin-2-yl]piperidine-2,6-dione (229 mg, 564 μmol, 1.0 eq) in THF (2 mL) was added. The mixture was stirred at −78° C. for 0.5 h. The mixture was stirred at 25° C. for 0.5 h. The reaction mixture was added 50 mL H₂O and extracted with EA (30 mL×3). The combined organic layers were filtered and concentrated under reduced pressure to give a residue. The residue was purified by prep-TLC (SiO₂, Petroleum ether: Ethyl acetate=0:1) to give compound tert-butyl 2-[[3-chloro-5-[[2-(2,6-dioxo-3-piperidyl)-4-fluoro-1-oxo-isoindolin-5-yl]methylcarbamoylamino]phenoxy]methyl]prop-2-enoate (0.2 g, 59% yield) as a colorless oil.

Step 2. To a solution of tert-butyl 2-[[3-chloro-5-[[2-(2,6-dioxo-3-piperidyl)-4-fluoro-1-oxo-isoindolin-5-yl] methylcarbamoylamino]phenoxy] methyl]prop-2-enoate (90 mg, 149.75 μmol, 1 eq) in DCM/TFA=1:1 (1 mL) was stirred at 25° C. for 0.5 h. The reaction mixture was concentrated under reduced pressure to remove solvent to give compound 2-[[3-chloro-5-[[2-(2,6-dioxo-3-piperidyl)-4-fluoro-1-oxo-isoindolin-5-yl] methylcarbamoylamino]phenoxy]methyl]prop-2-enoic acid (81 mg, 99% yield) as a colorless oil.

Step 3. To a solution of 2-[[3-chloro-5-[[2-(2,6-dioxo-3-piperidyl)-4-fluoro-1-oxo-isoindolin-5-yl]methylcarbamoylamino]phenoxy]methyl]prop-2-enoic acid (80 mg, 147 μmol, 1.0 eq), 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (95 mg, 147 μmol, 1.0 eq, TFA salt) in DMF (0.5 mL) was added DIEA (57 mg, 440 μmol, 77 μL, 3.0 eq) and T₃P (140 mg, 220 μmol, 50% purity, 1.5 eq). The mixture was stirred at 25° C. for 12 h. The residue was purified by prep-HPLC (column: Waters Xbridge 150×25 mm×5 um; mobile phase: [water (NH₄HCO₃)-ACN]; B %: 55%-85%, 8 min) to give final product compound, 1-[3-chloro-5-[2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carbonyl]allyloxy]phenyl]-3-[[2-(2,6-dioxo-3-piperidyl)-4-fluoro-1-oxo-isoindolin-5-yl]methyl]urea (17 mg, 11% yield) as a yellow solid. ¹H NMR (400 MHz, CD3OD): δ 7.80 (d, J=7.70 Hz, 1H), 7.66 (d, J=8.80 Hz, 1H), 7.43-7.57 (m, 4H), 7.25 (s, 1H), 7.15-7.23 (m, 1H), 6.95 (s, 1H), 6.66 (d, J=1.83 Hz, 1H), 5.66-5.80 (m, 1H), 5.43-5.60 (m, 1H), 5.04-5.22 (m, 1H), 4.86-4.98 (m, 5H), 4.07-4.60 (m, 8H), 3.52-3.77 (m, 2H), 2.44-3.23 (m, 16H), 2.03-2.21 (m, 2H), 1.61-1.88 (m, 3H), 1.30 (d, J=6.48 Hz, 2H), 1.19 (s, 1H).

### Example 12—Synthesis of Compound 1-(2-((2-(3-(4-amino-3-(4-phenoxyphenyl)-1H-pyrazolo[3,4-d]pyrimidin-1-yl)piperidine-1-carbonyl)allyl)oxy)-3-chlorophenyl)-3-((2-(2,6-dioxopiperidin-3-yl)-1-oxoisoindolin-5-yl)methyl)urea

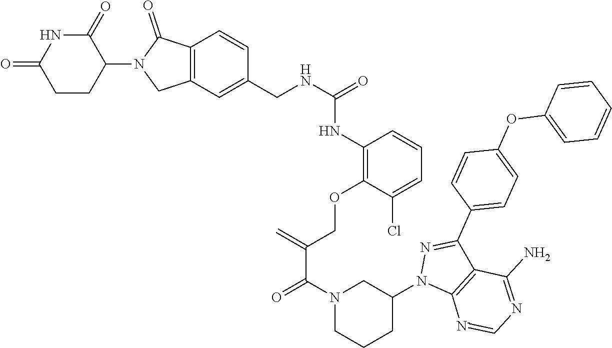

To a solution of Compound AS (40 mg, 0.075 mmol) and Compound AR (39.9 mg, 0.090 mmol) in DMF (1.0 mL) was added K₂CO₃ (104 mg, 0.75 mmol). The reaction mixture was stirred at rt for 1 h. The crude product was purified by reversed phase purification (C18) using a gradient of 0-80% MeCN in water (containing 0.1% formic acid) to afford compound V˜57 (39 mg, 58%) as a solid. LC-MS Method C: MS (ES⁺): Rₜ=3.49 min, m/z=895.3 [M+H]⁺. ¹H NMR (DMSO-d₆, 400 MHz): δ_{H} 10.98 (1H, s), 8.42-8.38 (1H, m), 8.23-8.14 (1H, m), 8.10 (1H, d, J=8.2 Hz), 7.75-7.55 (3H, m), 7.39-7.48 (4H, nm), 7.25-7.16 (m, 2H), 7.11 (4H, m), 7.05-6.95 (2H, m) 5.72-5.62 (1H, m), 5.50-5.42 (1H, m), 5.09 (1H, d, J=12.3 Hz), 4.82 (1H, br s), 4.64 (1H, br s), 4.51-4.19 (6H, m), 3.80 (1H, m), 3.27 (1H, m), 2.84-3.00 (2H, m), 2.57 (1H, m), 2.34-2.11 (3H, m), 2.00-1.85 (2H, m), 1.75-1.55 (1H, m).

### Example 13—Synthesis of Additional Compounds

The following compounds were prepared based on procedures described above for synthesis of compound V-57.

| Compound | | Starting |
|---|---|---|
| No. | Chemical Structure | Material |
| I-51 | 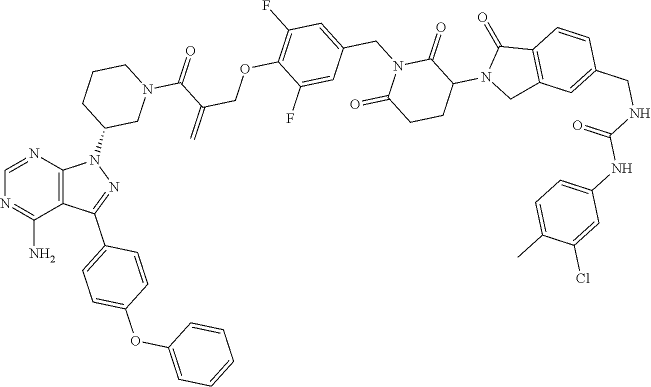 [SMILES: C=C(COc1c(F)cc(CN2C(=O)CCC(N3Cc4cc(CNC(=O)Nc5ccc(C)c(Cl)c5)ccc4C3=O)C2=O)cc1F)C(=O)N1CCC[C@@H](n2nc(-c3ccc(Oc4ccccc4)cc3)c3c(N)ncnc32)C1] | AR, AT |
| I-60 | 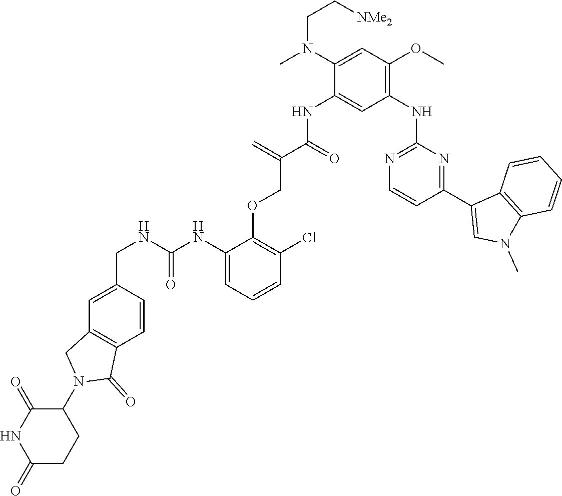 [SMILES: C=C(COc1c(Cl)cccc1NC(=O)NCc1ccc2c(c1)CN(C1CCC(=O)NC1=O)C2=O)C(=O)Nc1cc(Nc2nccc(-c3cn(C)c4ccccc34)n2)c(OC)cc1N(C)CCN(C)C] | AU, AS |
| I-62 | 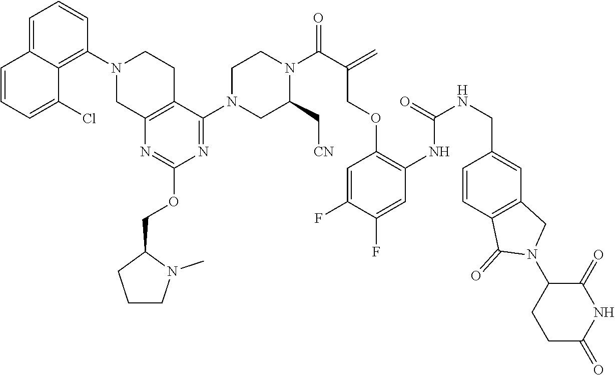 [SMILES: C=C(COc1cc(F)c(F)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] | AV, AW |
| I-63 | 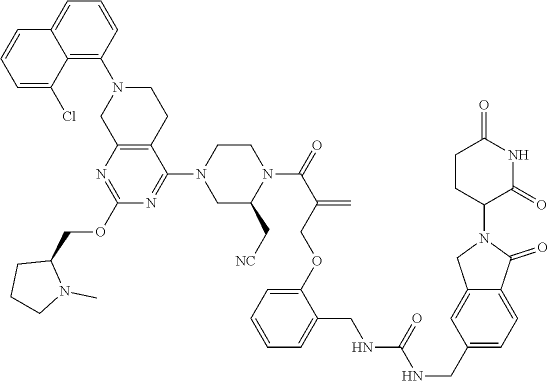 [SMILES: C=C(COc1ccccc1CNC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] | AV, AX |
| I-75 | 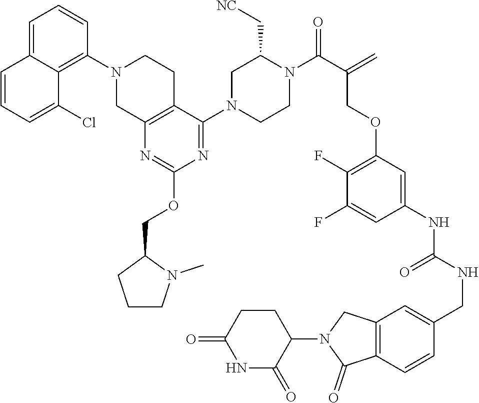 [SMILES: C=C(COc1cc(NC(=O)NCc2ccc3c(c2)CN(C2CCC(=O)NC2=O)C3=O)cc(F)c1F)C(=O)N1CCN(c2nc(OC[C@@H]3CCCN3C)nc3c2CCN(c2cccc4cccc(Cl)c24)C3)C[C@@H]1CC#N] | AV, AY |

Mass spectroscopic data and retention time in the LC-MS for the above compounds is provided in the following table.

| | | RT in LC-MS | LC-MS |
|---|---|---|---|
| Compound No. | MH+ | (min) | method |
| I-51 | 1035.4 | 1.93 | Method B |
| I-60 | 477.6, 954.2 | 1.756 | AB25 |
| I-62 | 521.7, 1042.3 | 2.039 | AB25 |
| I-63 | 1020.7 | 2.559 | AB05 |
| I-75 | 521.8, 1042.4 | 2.019 | AB25 |

### Example 14—Synthesis of Additional Compounds

Compounds in the following table were prepared based on procedure described above.

| Com- | |
|---|---|
| pound | |
| No. | Chemical Structure |
| I-78 | 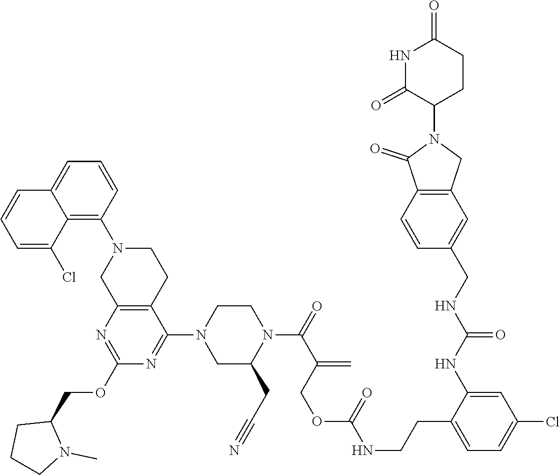 [SMILES: C=C(COC(=O)NCCc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-79 | 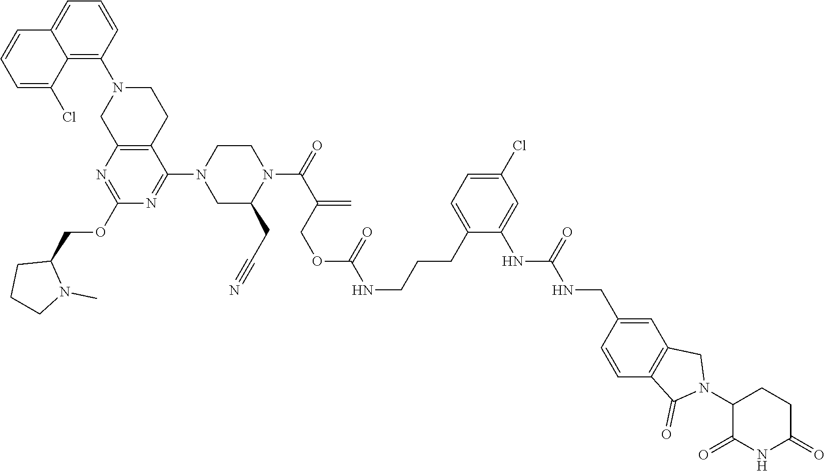 [SMILES: C=C(COC(=O)NCCCc1ccc(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| II-192 | 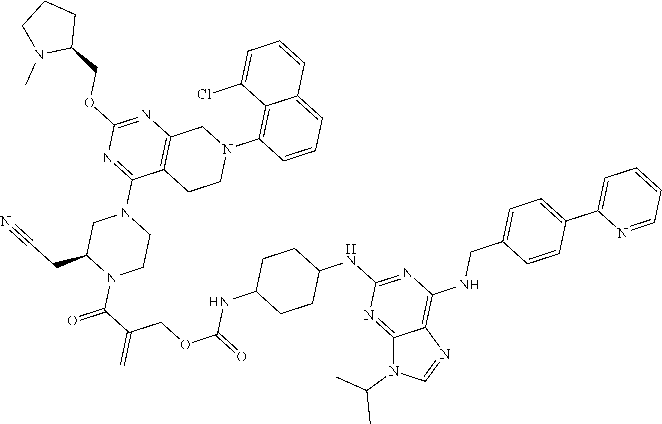 [SMILES: C=C(COC(=O)NC1CCC(Nc2nc(NCc3ccc(-c4ccccn4)cc3)c3ncn(C(C)C)c3n2)CC1)C(=O)N1CCN(c2nc(OC[C@@H]3CCCN3C)nc3c2CCN(c2cccc4cccc(Cl)c24)C3)C[C@@H]1CC#N] |
| I-87 | 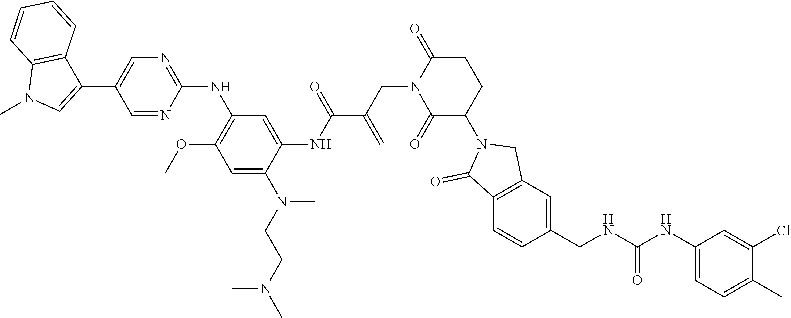 [SMILES: C=C(CN1C(=O)CCC(N2Cc3cc(CNC(=O)Nc4ccc(C)c(Cl)c4)ccc3C2=O)C1=O)C(=O)Nc1cc(Nc2ncc(-c3cn(C)c4ccccc34)cn2)c(OC)cc1N(C)CCN(C)C] |
| II-191 | 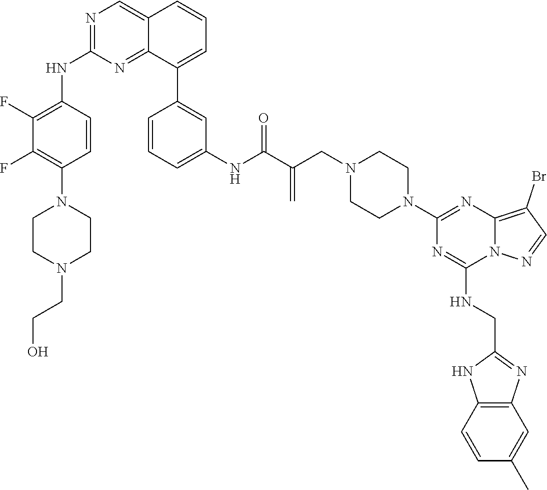 [SMILES: C=C(CN1CCN(c2nc(NCc3nc4cc(C)ccc4[nH]3)n3ncc(Br)c3n2)CC1)C(=O)Nc1cccc(-c2cccc3cnc(Nc4ccc(N5CCN(CCO)CC5)c(F)c4F)nc23)c1] |
| I-84 | 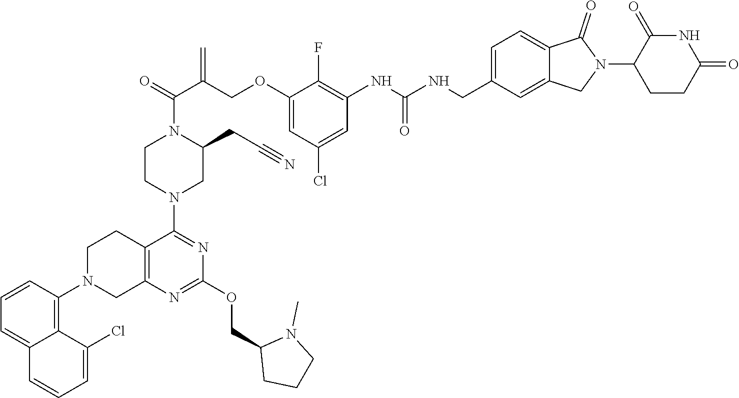 [SMILES: C=C(COc1cc(Cl)cc(NC(=O)NCc2ccc3c(c2)CN(C2CCC(=O)NC2=O)C3=O)c1F)C(=O)N1CCN(c2nc(OC[C@@H]3CCCN3C)nc3c2CCN(c2cccc4cccc(Cl)c24)C3)C[C@@H]1CC#N] |
| I-85 | 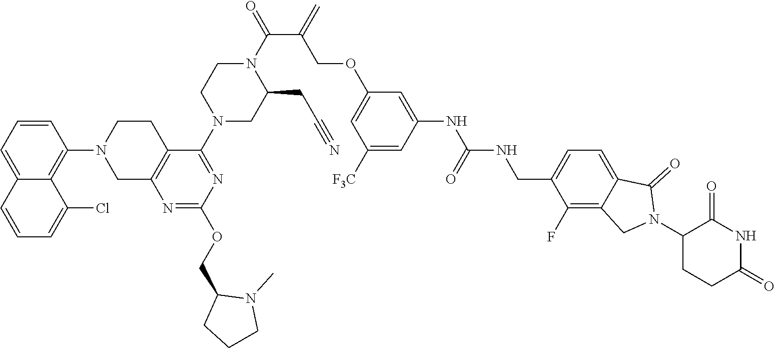 [SMILES: C=C(COc1cc(NC(=O)NCc2ccc3c(c2F)CN(C2CCC(=O)NC2=O)C3=O)cc(C(F)(F)F)c1)C(=O)N1CCN(c2nc(OC[C@@H]3CCCN3C)nc3c2CCN(c2cccc4cccc(Cl)c24)C3)C[C@@H]1CC#N] |
| I-103 | 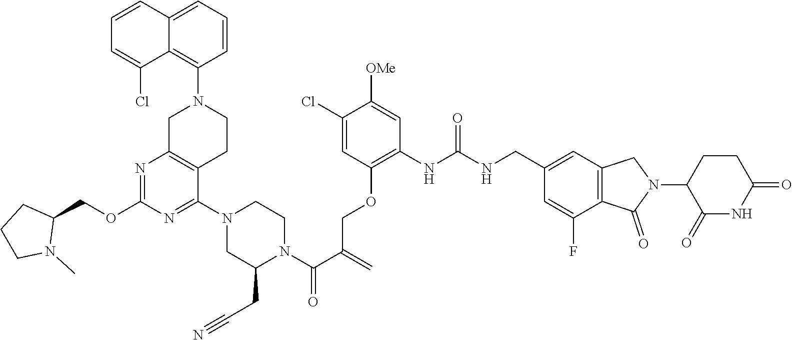 [SMILES: C=C(COc1cc(Cl)c(OC)cc1NC(=O)NCc1cc(F)c2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-104 | 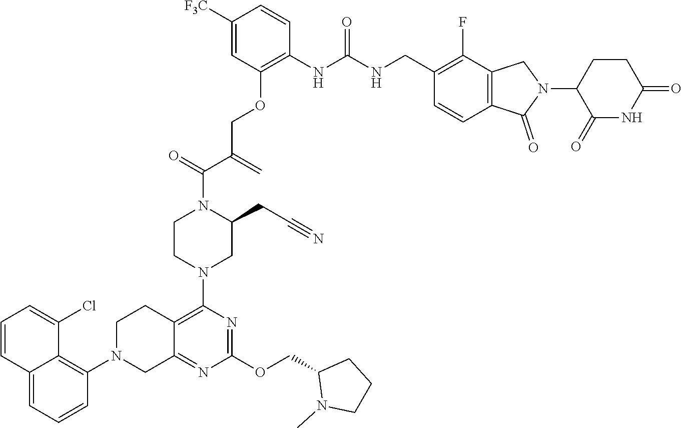 [SMILES: C=C(COc1cc(C(F)(F)F)ccc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-105 | 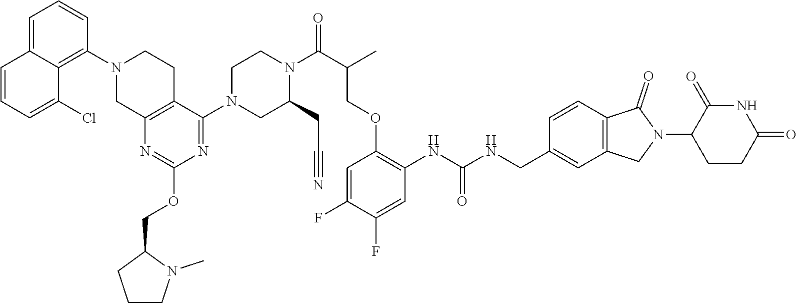 [SMILES: CC(COc1cc(F)c(F)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-111 | 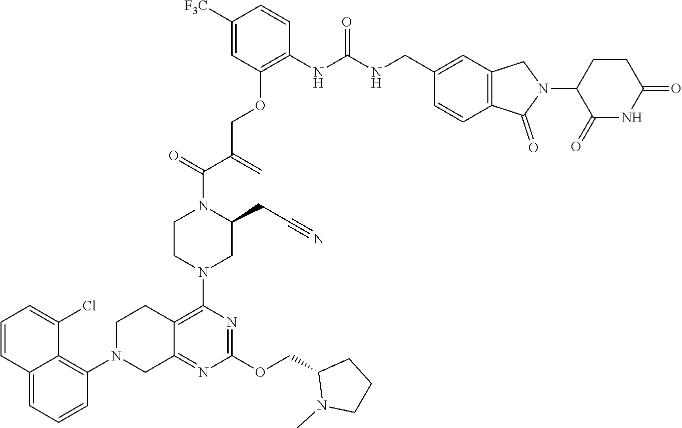 [SMILES: C=C(COc1cc(C(F)(F)F)ccc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-93 | 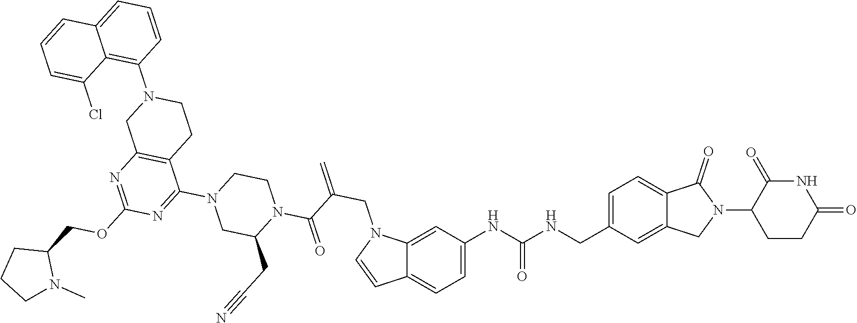 [SMILES: C=C(Cn1ccc2ccc(NC(=O)NCc3ccc4c(c3)CN(C3CCC(=O)NC3=O)C4=O)cc21)C(=O)N1CCN(c2nc(OC[C@@H]3CCCN3C)nc3c2CCN(c2cccc4cccc(Cl)c24)C3)C[C@@H]1CC#N] |
| I-83 | 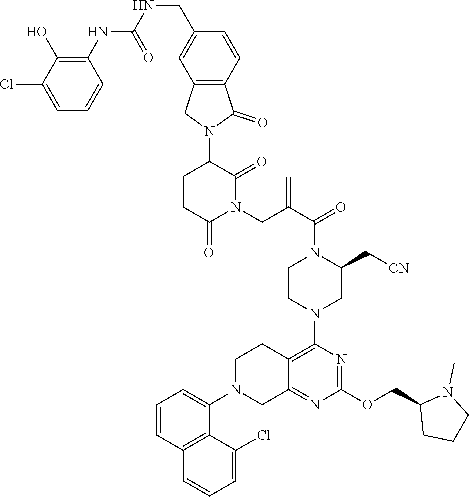 [SMILES: C=C(CN1C(=O)CCC(N2Cc3cc(CNC(=O)Nc4cccc(Cl)c4O)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-94 | 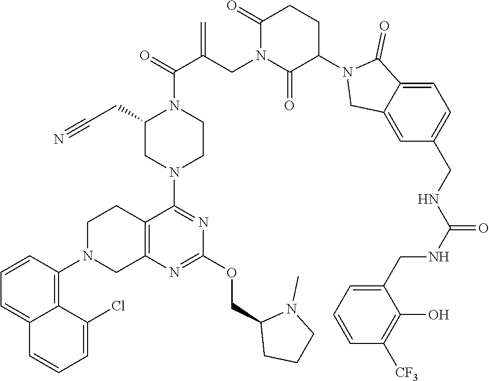 [SMILES: C=C(CN1C(=O)CCC(N2Cc3cc(CNC(=O)NCc4cccc(C(F)(F)F)c4O)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-107 | 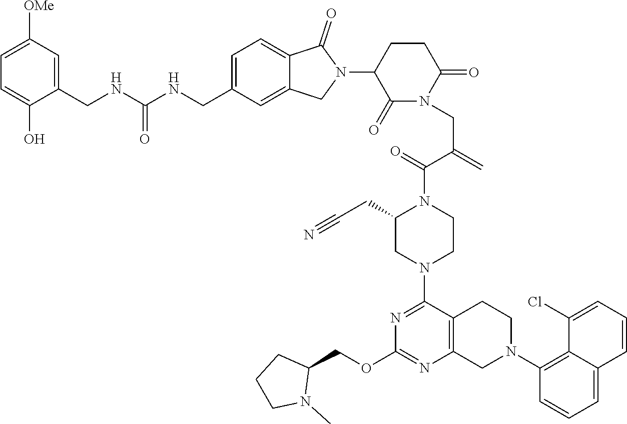 [SMILES: C=C(CN1C(=O)CCC(N2Cc3cc(CNC(=O)NCc4cc(OC)ccc4O)ccc3C2=O)C1=O)C(=O)N1CCN(c2nc(OC[C@@H]3CCCN3C)nc3c2CCN(c2cccc4cccc(Cl)c24)C3)C[C@@H]1CC#N] |
| I-99 | 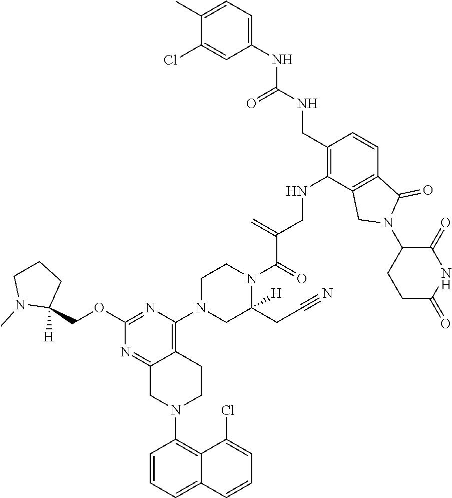 [SMILES: C=C(CNc1c(CNC(=O)Nc2ccc(C)c(Cl)c2)ccc2c1CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| II-188 | 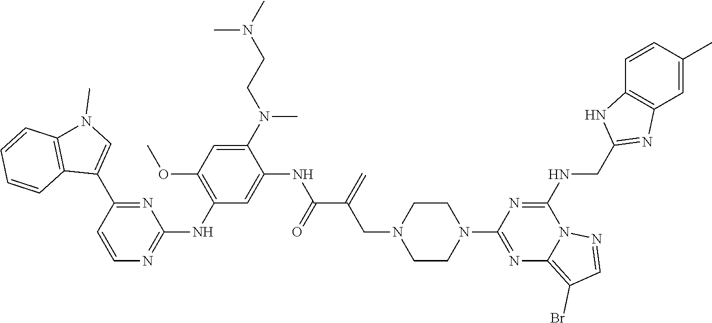 [SMILES: C=C(CN1CCN(c2nc(NCc3nc4cc(C)ccc4[nH]3)n3ncc(Br)c3n2)CC1)C(=O)Nc1cc(Nc2nccc(-c3cn(C)c4ccccc34)n2)c(OC)cc1N(C)CCN(C)C] |
| II-190 | 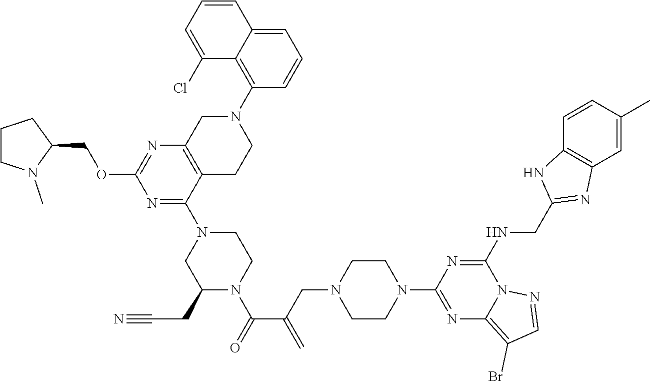 [SMILES: C=C(CN1CCN(c2nc(NCc3nc4cc(C)ccc4[nH]3)n3ncc(Br)c3n2)CC1)C(=O)N1CCN(c2nc(OC[C@@H]3CCCN3C)nc3c2CCN(c2cccc4cccc(Cl)c24)C3)C[C@@H]1CC#N] |
| I-80 | 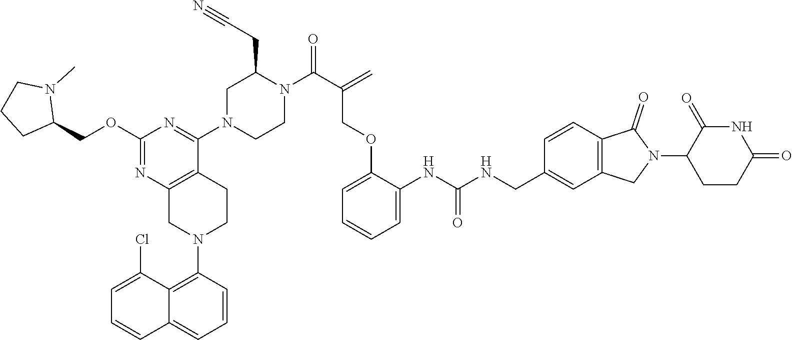 [SMILES: C=C(COc1ccccc1NC(=O)NCc1ccc2c(c1)CN(C1CCC(=O)NC1=O)C2=O)C(=O)N1CCN(c2nc(OC[C@H]3CCCN3C)nc3c2CCN(c2cccc4cccc(Cl)c24)C3)C[C@H]1CC#N] |
| I-81 | 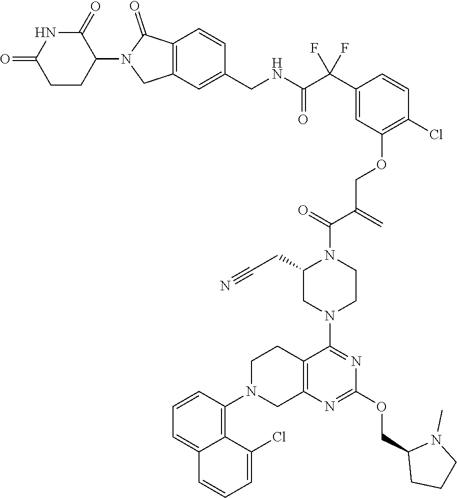 [SMILES: C=C(COc1cc(C(F)(F)C(=O)NCc2ccc3c(c2)CN(C2CCC(=O)NC2=O)C3=O)ccc1Cl)C(=O)N1CCN(c2nc(OC[C@@H]3CCCN3C)nc3c2CCN(c2cccc4cccc(Cl)c24)C3)C[C@@H]1CC#N] |
| I-82 | 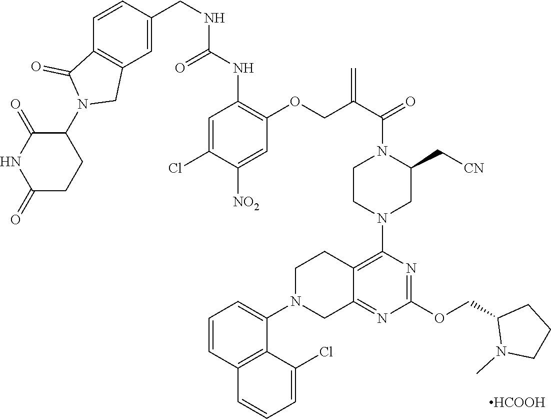 [SMILES: C=C(COc1cc([N+](=O)[O-])c(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N ; "·HCOOH"] |
| I-86 | 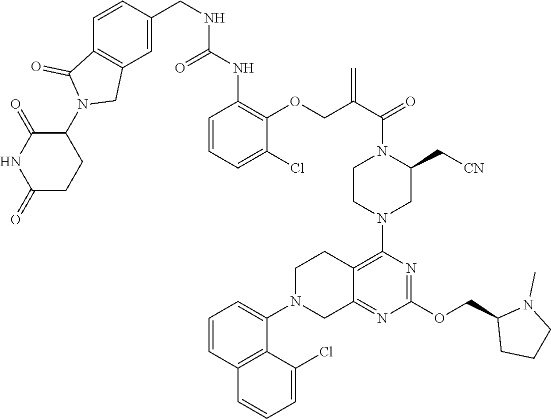 [SMILES: C=C(COc1c(Cl)cccc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-88 | 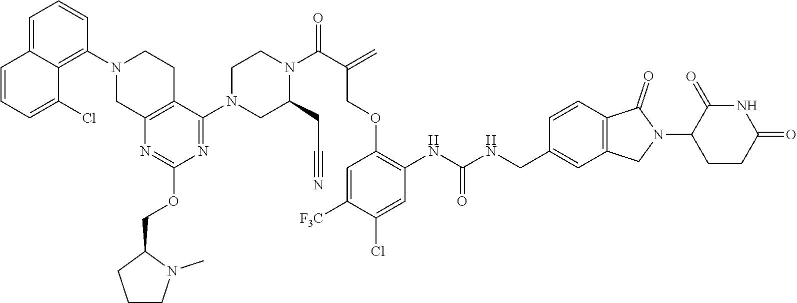 [SMILES: C=C(COc1cc(C(F)(F)F)c(Cl)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-89 | 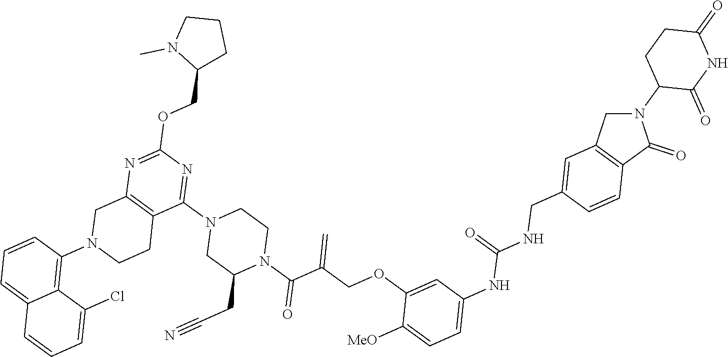 [SMILES: C=C(COc1cc(NC(=O)NCc2ccc3c(c2)CN(C2CCC(=O)NC2=O)C3=O)ccc1OC)C(=O)N1CCN(c2nc(OC[C@@H]3CCCN3C)nc3c2CCN(c2cccc4cccc(Cl)c24)C3)C[C@@H]1CC#N] |
| I-90 | 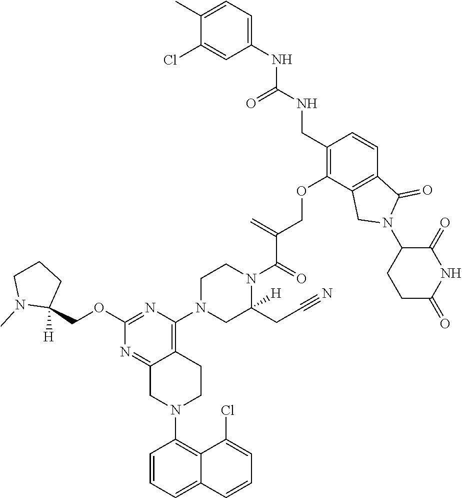 [SMILES: C=C(COc1c(CNC(=O)Nc2ccc(C)c(Cl)c2)ccc2c1CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-91 | 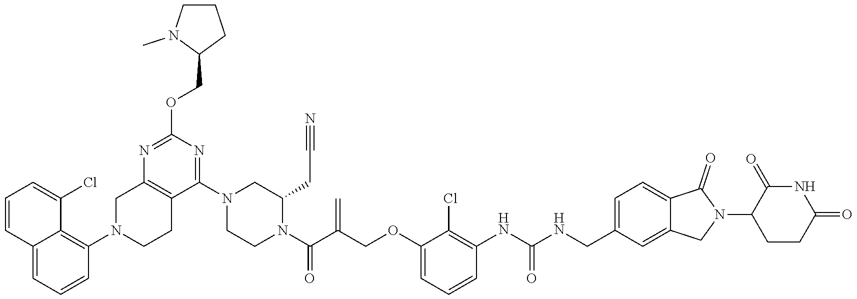 [SMILES: C=C(COc1cccc(NC(=O)NCc2ccc3c(c2)CN(C2CCC(=O)NC2=O)C3=O)c1Cl)C(=O)N1CCN(c2nc(OC[C@@H]3CCCN3C)nc3c2CCN(c2cccc4cccc(Cl)c24)C3)C[C@@H]1CC#N] |
| I-92 | 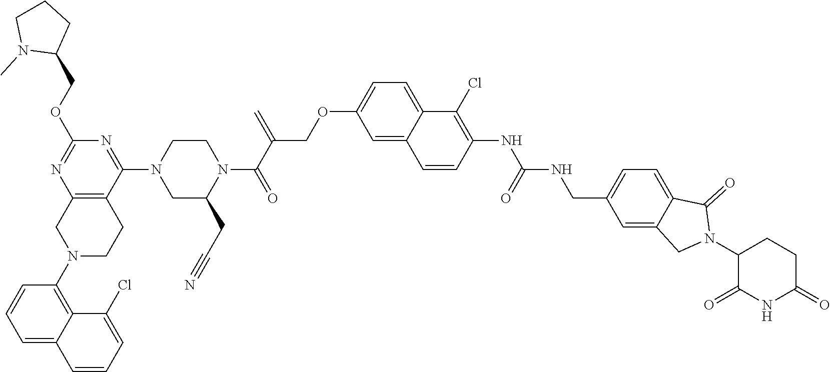 [SMILES: C=C(COc1ccc2c(Cl)c(NC(=O)NCc3ccc4c(c3)CN(C3CCC(=O)NC3=O)C4=O)ccc2c1)C(=O)N1CCN(c2nc(OC[C@@H]3CCCN3C)nc3c2CCN(c2cccc4cccc(Cl)c24)C3)C[C@@H]1CC#N] |
| I-95 | 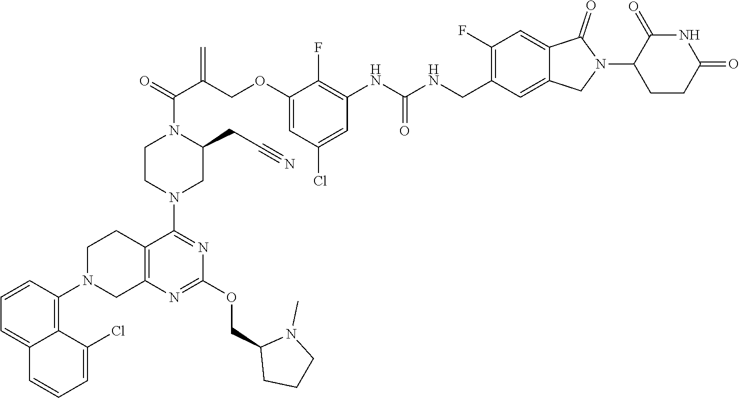 [SMILES: C=C(COc1cc(Cl)cc(NC(=O)NCc2cc3c(cc2F)C(=O)N(C2CCC(=O)NC2=O)C3)c1F)C(=O)N1CCN(c2nc(OC[C@@H]3CCCN3C)nc3c2CCN(c2cccc4cccc(Cl)c24)C3)C[C@@H]1CC#N] |
| I-96 | 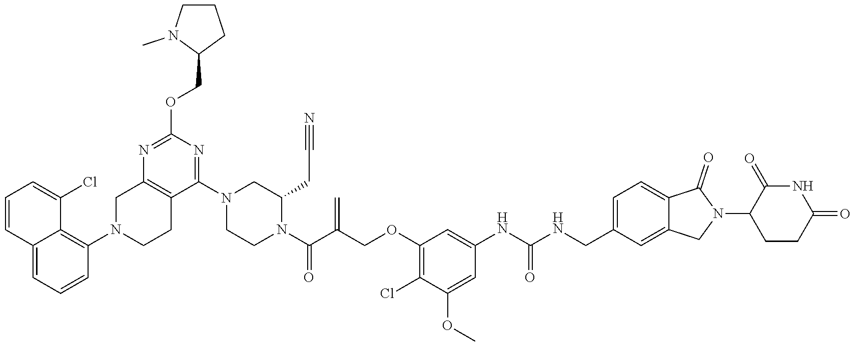 [SMILES: C=C(COc1cc(NC(=O)NCc2ccc3c(c2)CN(C2CCC(=O)NC2=O)C3=O)cc(OC)c1Cl)C(=O)N1CCN(c2nc(OC[C@@H]3CCCN3C)nc3c2CCN(c2cccc4cccc(Cl)c24)C3)C[C@@H]1CC#N] |
| I-97 | 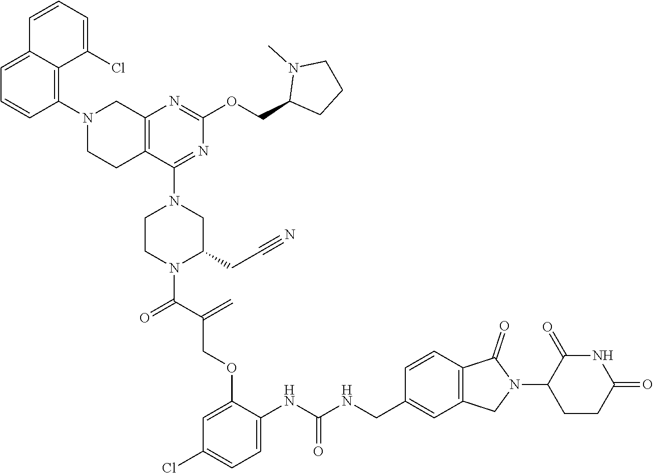 [SMILES: C=C(COc1cc(Cl)ccc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-98 | 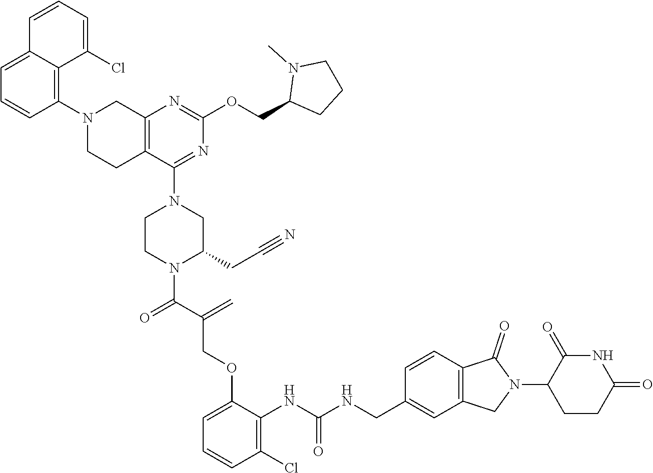 [SMILES: C=C(COc1cccc(Cl)c1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-100 | 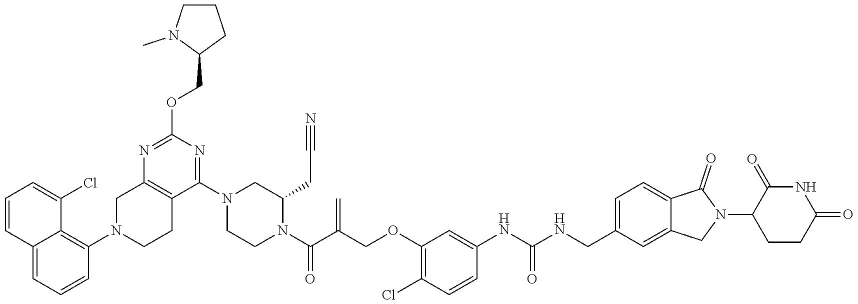 [SMILES: C=C(COc1cc(NC(=O)NCc2ccc3c(c2)CN(C2CCC(=O)NC2=O)C3=O)ccc1Cl)C(=O)N1CCN(c2nc(OC[C@@H]3CCCN3C)nc3c2CCN(c2cccc4cccc(Cl)c24)C3)C[C@@H]1CC#N] |
| I-101 | 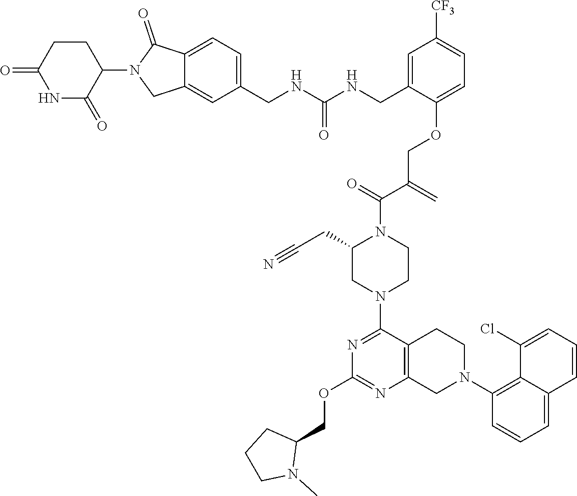 [SMILES: C=C(COc1ccc(C(F)(F)F)cc1CNC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-102 | 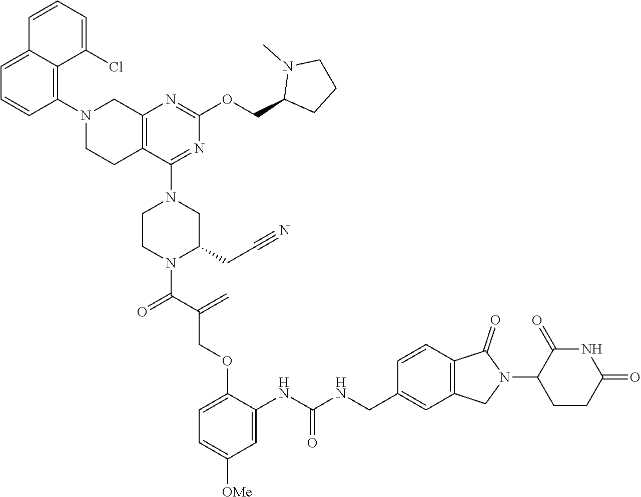 [SMILES: C=C(COc1ccc(OC)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-108 | 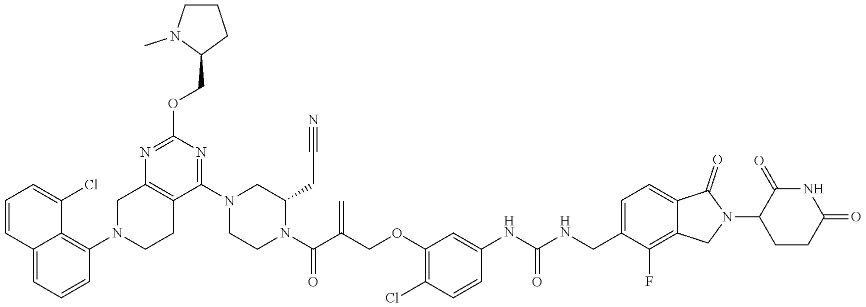 [SMILES: C=C(COc1cc(NC(=O)NCc2ccc3c(c2F)CN(C2CCC(=O)NC2=O)C3=O)ccc1Cl)C(=O)N1CCN(c2nc(OC[C@@H]3CCCN3C)nc3c2CCN(c2cccc4cccc(Cl)c24)C3)C[C@@H]1CC#N] |
| I-109 | 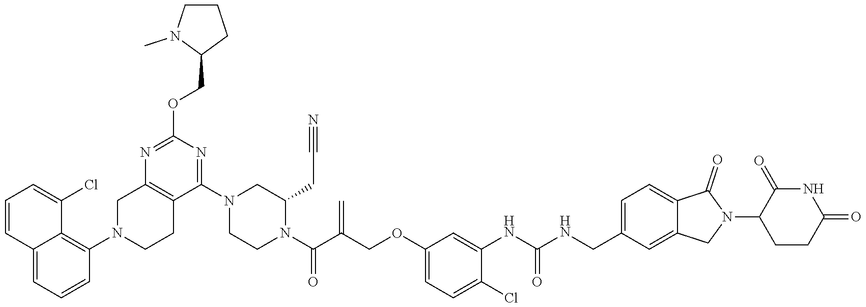 [SMILES: C=C(COc1ccc(Cl)c(NC(=O)NCc2ccc3c(c2)CN(C2CCC(=O)NC2=O)C3=O)c1)C(=O)N1CCN(c2nc(OC[C@@H]3CCCN3C)nc3c2CCN(c2cccc4cccc(Cl)c24)C3)C[C@@H]1CC#N] |
| I-110 | 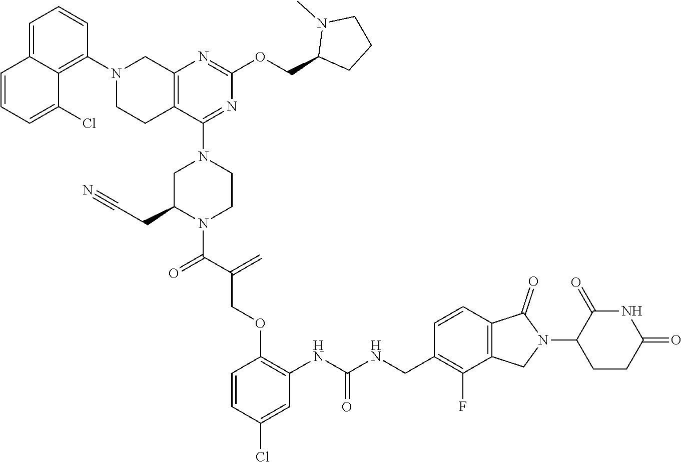 [SMILES: C=C(COc1ccc(Cl)cc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-112 | 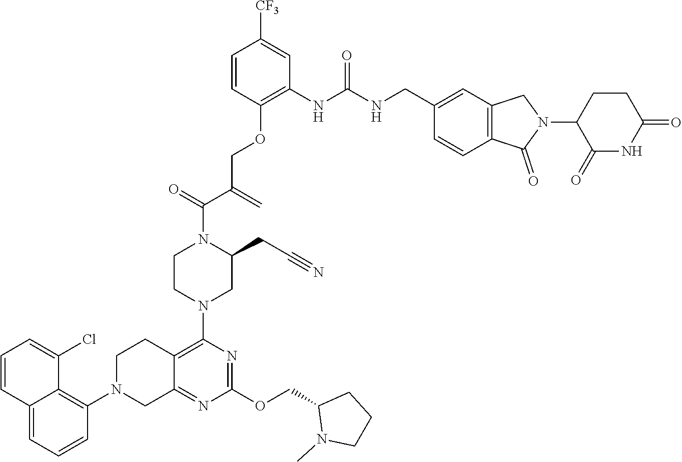 [SMILES: C=C(COc1ccc(C(F)(F)F)cc1NC(=O)NCc1ccc2c(c1)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-113 | 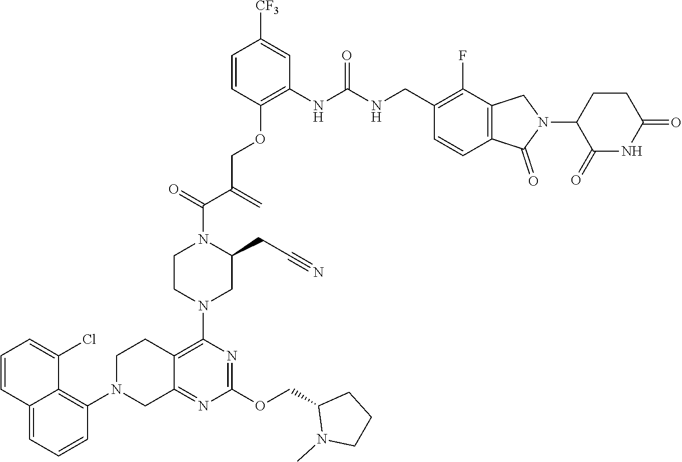 [SMILES: C=C(COc1ccc(C(F)(F)F)cc1NC(=O)NCc1ccc2c(c1F)CN(C1CCC(=O)NC1=O)C2=O)C(=O)N1CCN(c2nc(OC[C@@H]3CCCN3C)nc3c2CCN(c2cccc4cccc(Cl)c24)C3)C[C@@H]1CC#N] |
| I-114 | 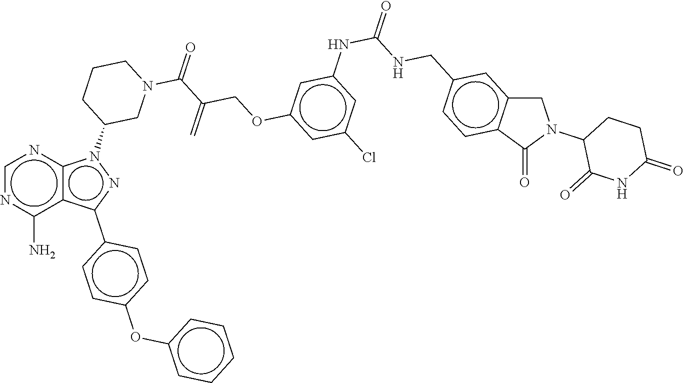 |
| I-115 | 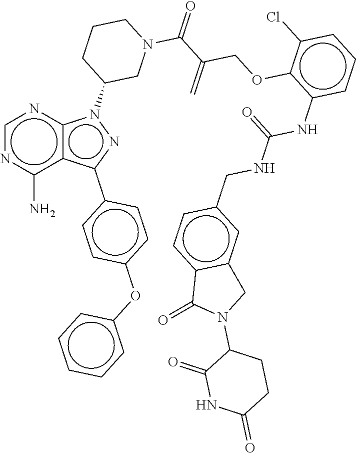 |
| I-116 | 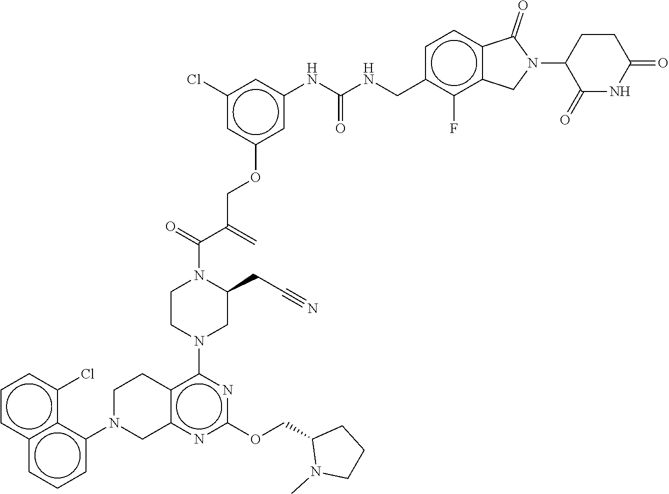 |
| II-193 | 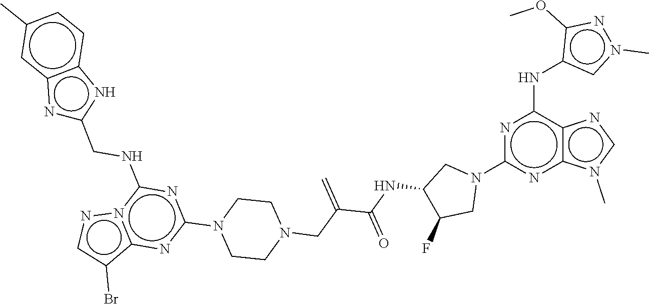 |

Mass spectroscopic data and retention time in the LC-MS for the above compounds, along with a description of starting material used to prepare the compounds, is provided in the following table.

| | | | Retention | |
|---|---|---|---|---|
| Compound | Starting | | Time in LC- | LC-MS |
| No. | Material | MH+ | MS (min) | method |
| I-78 | F, AZ | 556.2, 1111.3 | 1.999 | AB25 |
| I-79 | F, AAA | 563.3, 1125.4 | 2.022 | AB25 |
| II-192 | F, AAB | 366.9, 549.8 | 1.82 | AB25 |
| I-87 | L, Y | 952.2 | 1.847 | AB25 |
| I-191 | AAI, AM | 493.5, 986.2 | 1.573 | AB25 |
| I-84 | AAM, K | 1058.5 | 2.674 | AB05 |
| I-85 | AO, K | 546.7, 1092.3 | 2.136 | AB25 |
| I-103 | AAZ, K | 544.7, 1088.2 | 2.062 | AB25 |
| I-104 | ABA, K | 546.8, 1092.2 | 2.151 | AB25 |
| I-105 | ABB, K | 522.8, 1044.3 | 2.051 | AB25 |
| I-111 | ABG, K | 537.7, 1074.3 | 2.16 | AB25 |
| I-93 | AAH, K | 1029.4 | 1.22 | Method B |
| I-83 | AV, AAD | 1040.4 | 1.31 | Method B |
| I-94 | AV, AAE | 1088.4 | 1.39 | Method B |
| I-107 | AAF, AAG | 1051.3 | 2.76 | Method A |
| I-99 | AV, AAC | 1053.7 | 2.284 | AB05 |
| II-188 | AU, D | 954.3 | 1.902 | AB10 |
| II-190 | AV, D | 1041.2 | 1.646 | AB25 |
| I-80 | AV, AAJ | 503.8, 1006.3 | 1.934 | AB25 |
| I-81 | AV, AAK | 1075.4 | 1.34 | Method C |
| I-82 | AV, AAL | 1085.4 | 1.35 | Method B |
| I-86 | AV, AAD | 1040.3 | 1.34 | Method B |
| I-88 | AV, AAN | 1109.3 | 2.221 | AB25 |
| I-89 | AV, AAO | 518.7, 1036.3 | 1.854 | AB25 |
| I-90 | AV, AAP | 527.8, 1054.2 | 2.056 | AB25 |
| I-91 | AV, AAQ | 520.7, 1040.2 | 1.918 | AB25 |
| I-92 | AV, AAR | 1090.6 | 2.415 | AB05 |
| I-95 | AV, AAS | 1051.3 | 2.7 | Method A |
| I-96 | AV, AAT | 535.8, 1070.2 | 1.956 | AB25 |
| I-97 | AV, AAU | 1040.7 | 2.459 | AB05 |
| I-98 | AV, AAV | 1040.6 | 1.283 | — |
| I-100 | AV, AAW | 520.7, 1040.2 | 1.96 | AB25 |
| I-101 | AV, AAX | 1088.4 | 1.32 | Method B |
| I-102 | AV, AAY | 1036.6 | 2.375 | AB05 |
| I-108 | AV, ABD | 530.9, 1060.5 | 1.592 | AB25 |
| I-109 | AV, ABE | 1041.4 | 1.532 | AB25 |
| I-110 | AV, ABF | 530.9, 1060.5 | 1.637 | AB25 |
| I-112 | AV, ABH | 537.7, 1074.3 | 2.136 | AB25 |
| I-113 | AV, ABI | 1092.2 | 2.183 | AB25 |
| I-114 | — | 895.2 | 2.163 | AB25 |
| I-115 | — | 895.3 | 3.49 | Method C |
| I-116 | — | 530.5, 1060.3 | 2.073 | AB25 |
| II-193 | — | 436.1, 869.2 | 2.023 | AB10 |

Additionally, compound I-98 was subjected to LC-MS analysis using Method D. Using Method D, compound 1-98 had a retention time of 1.756 min and an observed mass of 520.8, 1040.2.

### Example 15—Cellular Growth Inhibition Assay for HEK293 Cells and HeLa Cells

An exemplary compound was tested for ability to inhibit the proliferation of HEK293 cells or HeLa cells. Experimental procedures and results are provided below.

### Part I—Experimental Procedure

HEK293 and HeLa cells were cultured in DMEM medium supplemented with 10% fetal bovine serum and 1% Penn/Strep. Cells were seeded in white 384-well plates at 500 cells/well in 25 μL complete medium. Following seeding, plates were spun at 300×g for three minutes and cultured at 37° C. with 5% CO₂ in a humidified tissue culture incubator.

After 24 hours, compounds were titrated in 100% DMSO and diluted in complete cell culture medium. A 25 μL aliquot of compound/media mixture was added to cells to bring total volume in the wells to 50 μL. DMSO alone was used as a negative control. Plates were then spun at 300×g for three minutes and stored at 37° C. with 5% CO₂ for three days.

On Day 0 and Day 3 of compound treatment, cell viability was quantified with CellTiter-Glo 2.0 reagent (Promega). After equilibrating microplates at room temperature for 30 minutes, 25 μL CellTiter-Glo 2.0 reagent was dispensed into each well to bring total volume to 75 μL. Plates were mixed on a shaker for 2 minutes at 500 rpm, followed by a 10-minute incubation at room temperature. Following a quick spin, luminescence readings were measured with an EnVision Plate Reader. Data was normalized to DMSO treated Day 0 and Day 3 readings. A four-parameter non-linear regression curve fit was applied to dose-response data in GraphPad Prism data analysis software to determine the half maximal growth inhibitory concentration (GI₅₀) for each compound.

### Part II—Results

Results are provided in Table 8 below. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 10 μM. The symbol “+” indicates a GI₅₀ greater than 10 μM. The symbol “N/A” indicates that no data was available.

TABLE 8

| | HEK293 | HeLa |
|---|---|---|
| Compound No. | (GI₅₀) | (GI₅₀) |
| I-4 | ++ | + |

### Example 16—Cellular Growth Inhibition Assay for HEK293 cells and HeLa cells

Exemplary compounds were tested for ability to inhibit the proliferation of HEK293 cells (ATCC CRL-1573) and HeLa cells (ATCC CCL-2). Experimental procedures and results are provided below.

### Part I—Experimental Procedure

HEK293 and HeLa cells were cultured in DMEM (Gibco 11995), supplemented with 10% Heat-inactivated FBS (Gibco A38400-01) and 1% Penicillin/Streptomycin (Gibco 15140-122) at 37° C. with 5% CO₂ in a humidified tissue culture incubator. Cells were seeded at 500 cells/well in 384-well, Poly-D-lysine-treated blackplates (Perkin Elmer 6007710) in 25 μL media lacking selection for 18-24 hours. Plates were spun at 300 g for 30 seconds and stored in the incubator overnight. After 24 hours, a 25 μL aliquot of compound-containing medium was added in each well, at a final top concentration of 10 μM test compound, with 3-fold dilutions, using DMSO alone as a negative control. Plates were then spun at 300×g for 3 minutes again and cultured at 37° C. with 5% CO₂. After 72 hours, cell viability was quantified with CellTiter-Glo 2.0 (Promega). After equilibrating cell plates at room temperature for 30 minutes, 25 μL CellTiter-Glo 2.0 reagent was dispensed into each well. Plates were mixed on a shaker for 2 minutes at 500 rpm, followed by a 10-minute incubation at room temperature. Luminescence readings were measured with an EnVision Plate Reader (Perkin Elmer). Data was normalized to DMSO treated cell wells. A four-parameter non-linear regression curve fit was applied to dose-response data in Prism to determine the half maximal growth inhibitory concentration (GI₅₀) of each compound.

### Part II—Results

Results are provided in Table 9 below. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 10 μM. The symbol “+” indicates a GI₅₀ greater than 10 μM. The symbol “N/A” indicates that no data was available.

TABLE 9

| | HEK293 | HeLa |
|---|---|---|
| Compound No. | (GI₅₀) | (GI₅₀) |
| I-30 | ++ | + |
| I-31 | + | + |
| I-32 | +++ | +++ |
| I-114 | +++ | ++ |
| I-33 | ++ | ++ |
| I-34 | ++++ | ++ |
| I-35 | ++ | ++ |
| I-36 | + | + |
| I-38 | + | + |
| I-39 | + | + |
| I-40 | + | + |
| I-41 | ++ | + |
| I-42 | ++ | ++ |
| I-43 | ++++ | +++ |
| I-44 | + | + |
| I-115 | ++ | +++ |
| I-45 | ++++ | ++ |
| I-46 | +++ | + |
| I-47 | ++++ | +++ |
| I-48 | ++ | + |
| I-49 | + | + |
| I-50 | + | + |
| I-51 | + | + |
| I-52 | +++ | ++ |
| I-53 | +++ | +++ |
| I-54 | +++ | ++ |
| I-116 | ++ | + |
| I-55 | +++ | ++ |
| I-56 | +++ | ++++ |
| I-57 | ++++ | ++ |
| I-58 | ++ | ++ |
| I-59 | ++++ | ++ |
| I-60 | +++ | ++ |
| I-61 | ++++ | ++ |
| I-62 | + | + |
| I-63 | + | + |
| I-64 | +++ | + |
| I-65 | ++ | + |
| I-66 | ++++ | +++ |
| I-67 | + | + |
| I-68 | ++++ | + |
| I-69 | ++++ | +++ |
| I-70 | ++ | + |
| I-71 | +++ | + |
| I-72 | ++ | ++ |
| I-73 | ++++ | ++ |
| I-74 | +++ | ++ |
| I-75 | + | + |
| I-76 | ++++ | +++ |
| I-77 | +++ | + |
| I-78 | + | N/A |
| I-79 | + | N/A |
| I-80 | + | N/A |
| I-81 | ++ | N/A |
| I-82 | + | N/A |
| I-83 | ++ | N/A |
| I-84 | ++++ | ++ |
| I-85 | ++ | + |
| I-86 | ++++ | +++ |
| I-87 | ++++ | ++++ |
| I-88 | ++++ | +++ |
| I-89 | + | + |
| I-90 | ++++ | + |
| I-91 | + | + |
| I-92 | ++++ | + |
| I-93 | +++ | +++ |
| I-94 | ++ | + |
| I-95 | +++ | ++ |
| I-96 | + | + |
| II-181 | + | + |
| II-193 | ++++ | ++++ |
| II-182 | ++ | ++ |
| II-183 | ++++ | ++++ |
| II-184 | ++++ | ++++ |
| II-185 | ++++ | +++ |
| V-4 | ++++ | +++ |
| II-186 | +++ | ++ |
| II-187 | ++++ | ++++ |
| II-188 | ++++ | ++++ |
| II-189 | +++ | +++ |
| II-190 | ++++ | ++++ |
| II-191 | ++++ | ++++ |
| II-192 | +++ | ++++ |

### Example 17—Assay for Inhibition of KRAS G12C

Exemplary compounds were tested for ability to inhibit KRas G12C. Experimental procedures and results are provided below.

### Part I—Experimental Procedure

KRAS target engagement (IC₅₀ determination) was performed using a KRAS G12C nucleotide exchange assay. Specifically, compounds were tested against KRAS G12C in 10-point concentration IC₅₀ mode with 3-fold serial dilution at a starting concentration of 10 μM. ARS1620 was used as a control.

Briefly, GST-tagged KRAS G12C (amino acids 2-169) was mixed with anti-GST Tb antibody (1.5× solution) and 10 μL was delivered to reaction wells. Compounds were delivered using an acoustic dispenser (Echo, Labcyte) and pre-incubated with protein for 1 hr at RT. KRAS/anti-GST Tb Ab/compound mixture was incubated for 1 hour at room temperature. A 3× solution of SOS1 (amino acids 564-1049) and GTP-DY-647P1 (GTP*) was prepared in reaction buffer. A 5 μL aliquot of SOS1/GTP* solution was added to reaction wells to initiate the exchange reaction. No-SOS1 reaction or max compound concentration was used as blank. The final concentrations of KRAS G12C, SOS1, and GTP* were 30 nM, 20 nM, and 0.15 μM, respectively. SOS1 mediated exchange of GDP to GTP-DY-647P was measured using HTRF (Ex/Em=(320-75/665-7.5; 615-8.5)) using an Envision Plate Reader (Perkin Elmer). IC₅₀ determination was performed using a Sigmoidal dose response (variable slope) equation.

### Part II—Results

Results showing inhibition of KRAS G12C by exemplary compounds are provided in Table 10 below. The symbol “++++” indicates a IC₅₀ less than 0.1 μM. The symbol “+++” indicates an IC₅₀ in the range of 0.1 μM to 5 μM. The symbol “++” indicates a IC₅₀ in the range of greater than 5 μM to 10 μM. The symbol “+” indicates a IC₅₀ greater than 10 μM. The symbol “N/A” indicates that no data was available.

TABLE 10

| Compound No. | IC₅₀ |
|---|---|
| I-2 | + |
| I-35 | ++++ |
| I-43 | + |
| I-44 | +++ |
| I-46 | ++++ |
| I-48 | ++++ |
| I-49 | ++++ |
| I-50 | ++++ |
| I-52 | ++++ |
| I-116 | ++++ |
| I-56 | +++ |
| I-57 | + |
| I-58 | ++ |
| I-62 | ++++ |
| I-63 | ++++ |
| I-64 | ++++ |
| I-65 | ++++ |
| I-66 | +++ |
| I-67 | +++ |
| I-68 | +++ |
| I-72 | ++++ |
| I-73 | ++++ |
| I-74 | ++++ |
| I-75 | ++++ |
| I-76 | +++ |
| I-77 | +++ |
| I-78 | ++++ |
| I-79 | ++++ |
| I-80 | ++++ |
| I-81 | ++++ |
| I-82 | +++ |
| I-83 | +++ |
| I-84 | + |
| I-85 | ++++ |
| I-86 | ++++ |
| I-88 | ++++ |
| I-89 | ++++ |
| I-90 | ++++ |
| II-190 | ++++ |
| II-186 | ++++ |
| II-187 | ++++ |

### Example 18—Assay for Binding Affinity to BTK

Exemplary compounds were tested for ability to bind to full-length BTK (amino acids 1-659; Accession Number NP_000052.1) expressed from mammalian cells. Compounds were tested using the KdELECT assay. Experimental procedures and results are provided below.

### Part I—Experimental Procedure

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17× PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing compound binding to BTK are provided in Table 11 below. The symbol “++++” indicates a K_{d} less than 0.05 μM. The symbol “+++” indicates an K_{d} in the range of 0.05 μM to 0.5 μM. The symbol “++” indicates a K_{d} in the range of greater than 0.5 M to 2.5 μM. The symbol “+” indicates a K_{d} greater than 2.5 μM. The symbol “N/A” indicates that no data was available.

TABLE 11

| Compound No. | K_{d} |
|---|---|
| I-3 | ++ |
| I-30 | +++ |
| I-31 | ++ |
| I-32 | +++ |
| I-114 | ++++ |
| I-38 | ++ |
| I-181 | + |

### Example 19—Assay for Binding Affinity to EGFR

Exemplary compounds were tested for ability to bind to EGFR WT (amino acids 669-1011, Accession Number NP_005219.2) or EGFR T790M, L858R (amino acids 669-1011 Accession Number NP_005219.2) expressed from bacteria or mammalian cells, respectively. Compounds were tested using the KdELECT assay at Eurofins Discovery. Experimental procedures and results are provided below.

### Part I—Experimental Procedure

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1x binding buffer (20% SeaBlock, 0.17× PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing compound binding to EGFR are provided in Table 12 below. The symbol “++++” indicates a Kd less than 0.05 μM. The symbol “+++” indicates an Kd in the range of 0.05 μM to 0.5 μM. The symbol “++” indicates a Kd in the range of greater than 0.5 M to 2.5 μM. The symbol “+” indicates a Kd greater than 2.5 μM. The symbol “N/A” indicates that no data was available.

TABLE 12

| Compound No. | T790M/L858R Kd | WT Kd |
|---|---|---|
| I-41 | + | + |
| I-42 | ++ | + |
| I-45 | ++++ | +++ |
| I-47 | ++++ | ++++ |
| I-54 | + | + |
| I-55 | ++ | + |
| I-59 | ++++ | ++++ |
| I-60 | ++++ | +++ |
| I-61 | +++ | ++ |
| I-87 | ++++ | + |
| II-193 | +++ | +++ |
| II-182 | + | + |
| II-184 | +++ | +++ |
| II-183 | +++ | +++ |
| II-185 | + | + |
| V-4 | + | + |
| II-188 | ++++ | ++++ |
| II-189 | + | + |
| II-191 | ++ | + |

### Example 20—Intact Mass Spec Target Engagement

Exemplary heterobifunctional compounds were evaluated for intact mass spectrometry target engagement against KRAS G12C, BTK, or EGFR T790M L858R (amino acids 669-1011; Accession Number NP_005219.2), separately. Experimental procedures and results are provided below.

### Part I—Experimental Procedure

Experimental procedures used when testing exemplary heterobifunctional compounds against KRAS G12C, BTK, or EGFR T790M L858R are provided below:

KRAS G12C: Samples were prepared using 5 μM of KRAS G12C C51S C80L C118S (amino acids 1-169) and 50 μM of heterobifunctional compound in a buffer containing 20 mM HEPES pH 7.5, 150 mM NaCl, and 1 mM TCEP. Protein was incubated with heterobifunctional compound at RT or 37° C. for the given amounts of time then flash-frozen and stored at −80° C. until analysis. Samples were transferred to HPLC vials and injected into Dionex UltiMate 3000 FLM HPLC system with a proprietary column (2.1×50 mm, 5 μm, 1000 Å) at 0.3 mL/minute in a column compartment at 50° C. and run on a Q Exactive Hybrid Quadrupole-Orbitrap Mass Spectrometer. Ion Max source with HESI-II probe were used, with a source voltage of 3.5 kV.

BTK: Samples were prepared using 2 μM of BTK (amino acids 387-659) and 20 μM of heterobifunctional compound in a buffer containing 20 mM HEPES pH 7.5, 150 mM NaCl, 1 mM TCEP. Protein was incubated with heterobifunctional compound at RT or 37° C. for the given amounts of time then flash-frozen and stored at −80° C. until analysis. Samples were transferred to HPLC vials and injected into Dionex UltiMate 3000 FLM HPLC system with a proprietary column (2.1×50 mm, 5 μm, 1000 Å) at 0.3 mL/minute in a column compartment at 50° C. and run on a Q Exactive Hybrid Quadrupole-Orbitrap Mass Spectrometer. Ion Max source with HESI-II probe were used, with a source voltage of 3.5 kV.

EGFR T790M L858R: Samples were prepared using 2 μM of EGFR T790M L858R (amino acids 696-1022) and 20 μM of heterobifunctional compound in a buffer containing 20 mM HEPES pH 7.5, 150 mM NaCl, and 1 mM TCEP. Protein was incubated with heterobifunctional compound at RT or 37° C. for the given amounts of time then flash-frozen and stored at −80° C. until analysis. Samples were transferred to HPLC vials and injected into Dionex UltiMate 3000 FLM HPLC system with a proprietary column (2.1×50 mm, 5 μm, 1000 Å) at 0.3 mL/minute in a column compartment at 50° C. and run on a Q Exactive Hybrid Quadrupole-Orbitrap Mass Spectrometer. Ion Max source with HESI-II probe were used, with a source voltage of 3.5 kV.

### Part II—Results

Results showing ability of exemplary heterobifunctional compounds to generate a modified target protein via intact mass spectrometry are shown in Table 13 below. The amount of different components identified by mass spectrometry at 24 hours post-reaction are listed in Table 14, wherein the different components include:
Un-modified Protein—this is starting protein material (e.g., KRAS G12C, BTK, or EGFR T790M L858R) that remained unchanged in the experiment.
Elimination-Conjugate Product—this is a conjugate formed by reaction of protein with the heterobifunctional compound to form a protein conjugate of the following general formula:

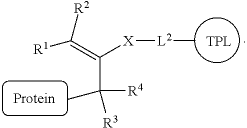

Notably, the effector protein ligand component of the heterobifunctional compound has been displaced by the protein.
Addition-Conjugate Product—this is a conjugate formed by reaction of protein with the heterobifunctional compound to form a protein conjugate of the following general formula:

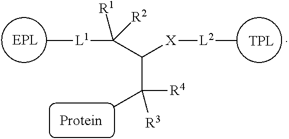

Notably, the effector protein ligand component of the heterobifunctional compound has not been displaced by the protein.
Other Material—this is material detected by mass spectroscopy, where the material is other than Un-modified Protein, Elimination-Conjugate Product, or Addition-Conjugate Product. Exemplary Other Material can include, for example, residual impurities in the protein starting material used to conduct the experiment.

TABLE 13

| | | Percent of | Percent of | |
|---|---|---|---|---|
| | Percent of | Elimination- | Addition- | Percent |
| Compound | Un-modified | Conjugate | Conjugate | Other |
| No. | Protein | Product | Product | Material |
| AI-2 | 2% | 83% | 5% | 10% |
| AI-4 | 4% | 72% | 24% | 0% |

Chemical Structures for compounds denoted in the above table are provided below:

| Compound No. | Chemical Structure |
|---|---|
| A1-2 | 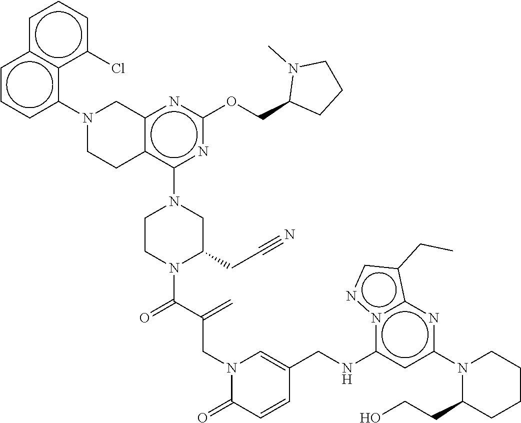 |
| A1-4 | 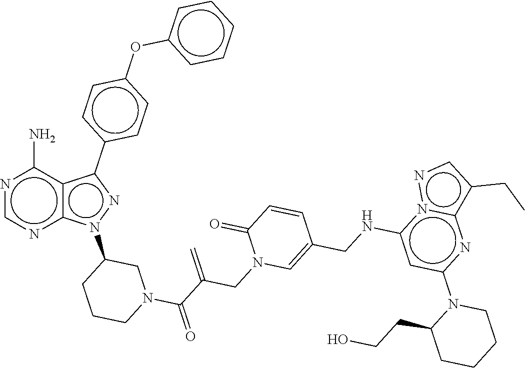 |

### Example 21—Release of Effector Protein Ligand from Heterobifunctional Compounds

Exemplary heterobifunctional compounds were evaluated for the ability to release an effector protein ligand in the presence of KRAS G12C, BTK, or EGFR T790M L858R target proteins. Experimental procedures and results are provided below.

### Part I—Experimental Procedure

Samples for mass spectrometry for KRAS G12C, BTK, and EGFR T790M L858R were prepared as described above for the intact mass spectrometry target engagement experiment, respectively, except rather than flash-freezing, 20 μL of sample was quenched with 50 μL of 100% acetonitrile. As well, a buffer-only control was used to detect latent release of effector protein ligand analyte. Internal standard (IS) solution consisted of 200 ng/mL propranolol and 50 ng/mL diclofenac in methanol:water 1:1 (v/v). A 4-point calibration curve (calibration standard (CS)) consisting of each analyte was made at final concentrations of 3 nM, 30 nM, 300 nM, and 3000 nM. Unknown samples were diluted such that the unknown analyte concentration was in the linear range of the calibration curve.

Samples for all proteins were prepared and analyzed from the following:

| | Blank | Blank + IS | CS | Sample |
|---|---|---|---|---|
| Unknown Lysate Sample (μL) | 0 | 0 | 0 | 20 |
| Blank Lysate (μL) | 20 | 20 | 20 | 0 |
| DMSO:ACN 1:1 (μL) | 40 | 20 | 0 | 20 |
| IS Soln (μL) | 0 | 20 | 20 | 20 |
| Std. Working Soln (μL) | 0 | 0 | 20 | 0 |
| Chilled Acetonitrile (μL) | 200 | 200 | 200 | 150 |
| Total Volume (μL) | 260 | 260 | 260 | 260 |

Samples were further analyzed with AB Sciex Triple Quad 5500 system coupled with Shimadzu Prominence HPLC. Instrument conditions are set forth below:

| HPLC | | Shimadzu LC-20AD Pumps and SIL-20AC Autosampler | | | | | | | |
|---|---|---|---|---|---|---|---|---|---|
| Instrument: | | | | | | | | | |
| Column: | Waters XBridge Phenyl, 3.5 μm, 1 × 50 mm | | | | | | SIL-20AC | Run Time | |
| | | | | | Flow | Diverting | Cooler | (min) | |
| MPA: | 0.1% Acetic Acid 1 mM NH₄OAc in | | | | Rate | to Waste | Temperature | Last gradient | |
| | Water | | | | (mL/min) | (min) | (° C.) | time point shown | |
| MPB: | 50 mM Acetic Acid in Acetonitrile | | | | 0.2 | 0-1 | NA | below | |
| Gradient | Time | 0 | 2.5 | 3.5 | 3.51 | 5.00 | | | |
| for | (min) | | | | | | | | |
| Compound | % B | 10 | 95 | 95 | 10 | 10 | | | |
| A1-1 | | | | | | | | | |
| AB Sciex | Ion | Scan | Polarity | CUR | CAD | IS | TEM | GS1 | GS2 |
| Triple | Source | Type | | | | | | | |
| Quad | Turbo | MRM | Positive | 20.00 | 6.00 | 5000.00 | 500.00 | 30.00 | 50.00 |
| 5500 | Spray | | | | | | | | |
| | | Q1 | Q3 | Time | DP | EP | CE | CXP | Injection |
| | | | | (msec) | | | | | Volume |
| | | | | | | | | | (μL) |

| | | | | | | | | |
|---|---|---|---|---|---|---|---|---|
| Compound A1-1 | 441.20 | 330.20 | 300.00 | 200.00 | 10.00 | 28.00 | 12.00 | 2.00 |
| Propranolol (IS) | 260.15 | 116.10 | 100.00 | 66.00 | 8.00 | 17.00 | 6.00 | NA |

Compound A1-1 has the following formula:

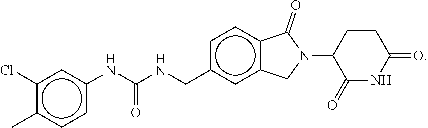

### Part II—Results

Results showing effector protein ligand release from a heterobifunctional compound that binds KRAS G12C are provided below.

| | Compound No. for | | | Concentration |
|---|---|---|---|---|
| | Compound | | | of Effector |
| Compound | Detected by Mass | | Time | Protein Ligand |
| No. | Spectrometry | Matrix | (h) | (nM) |
| A1-2 | A1-3 | Buffer | 0 | 55.8 |
| | | | 2 | 85.3 |
| | | | 8 | 166 |
| | | | 24 | 406 |
| A1-2 | A1-3 | Buffer + | 0 | 328 |
| | | Protein | 2 | 556 |
| | | | 8 | 1170 |
| | | | 24 | 2020 |

Chemical Structures for compounds denoted in the above table are provided below:

| Compound No. | Chemical Structure |
|---|---|
| A1-2 | 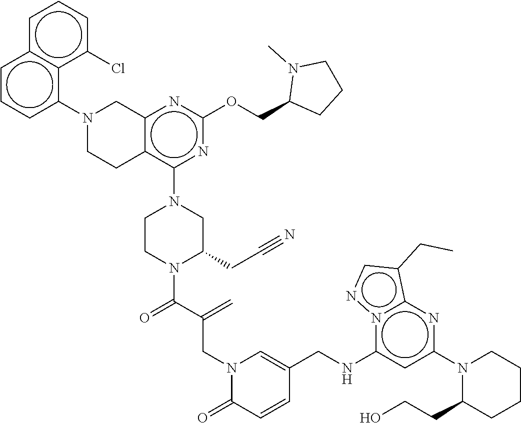 |
| A1-3 | 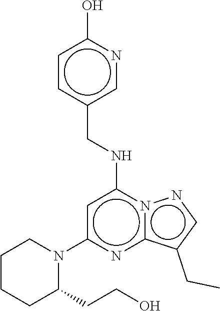 |

Results showing effector protein ligand release from heterobifunctional compounds that bind BTK are provided below.

| | Compound No. for | | | Concentration |
|---|---|---|---|---|
| | Compound | | | of Effector |
| Compound | Detected by Mass | | Time | Protein Ligand |
| No. | Spectrometry | Matrix | (h) | (nM) |
| A1-4 | A1-3 | Buffer | 0 | 4.16 |
| | | | 1 | 4 |
| | | | 4 | 4.49 |
| | | | 22 | 10.4 |
| A1-4 | A1-3 | Buffer + | 0 | 17.8 |
| | | Protein | 1 | 281 |
| | | | 4 | 506 |
| | | | 22 | 460 |
| A1-5 | A1-3 | Buffer | 0 | 44.9 |
| | | | 1 | 49 |
| | | | 4 | 52.4 |
| | | | 22 | 80.5 |
| A1-5 | A1-3 | Buffer + | 0 | 51.8 |
| | | Protein | 1 | 72.3 |
| | | | 4 | 118 |
| | | | 22 | 164 |
| A1-6 | A1-7 | Buffer | 0 | 104 |
| | | | 1 | 92.6 |
| | | | 4 | 66.4 |
| | | | 22 | 86.4 |
| A1-6 | A1-7 | Buffer + | 0 | 790 |
| | | Protein | 1 | 797 |
| | | | 4 | 641 |
| | | | 22 | 770 |

Chemical Structures for compounds denoted in the above table are provided below:

| Compound No. | Chemical Structure |
|---|---|
| A1-3 | 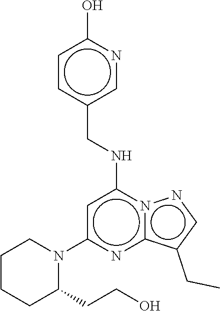 |
| A1-4 | 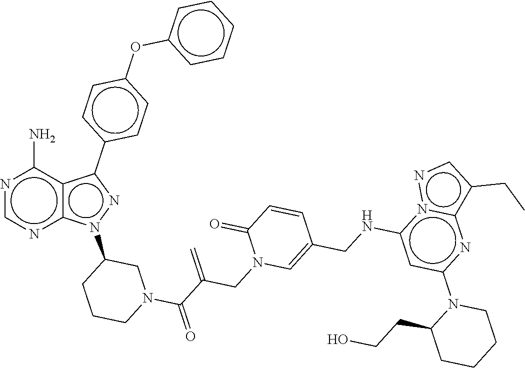 |
| A1-5 | 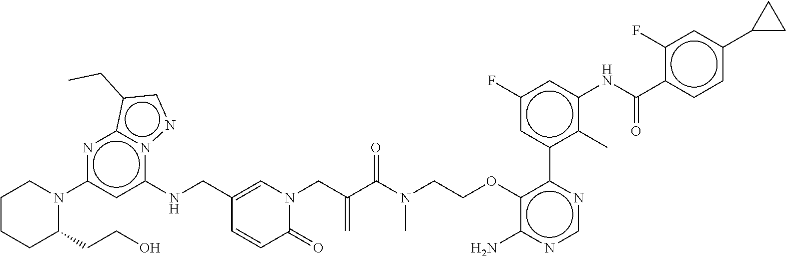 |
| A1-6 | 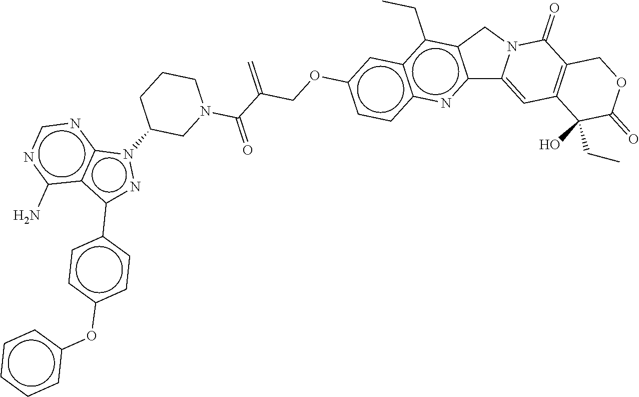 |
| A1-7 | 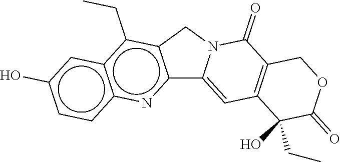 |

Results showing effector protein ligand release from a heterobifunctional compound that binds EGFR T790M L858R are provided below.

| | Compound No. | | | Concentration |
|---|---|---|---|---|
| | for Compound | | | of Effector |
| Compound | Detected by Mass | Buffer/ | Time | Protein Ligand |
| No. | Spectrometry | Protein | (h) | (nM) |
| A1-8 | A1-3 | Buffer | 0 | 894 |
| | | | 2 | 4060 |
| | | | 8 | 7240 |
| | | | 24 | 8260 |
| A1-8 | A1-3 | Buffer + | 0 | 889 |
| | | Protein | 2 | 5180 |
| | | | 8 | 6910 |
| | | | 24 | 9110 |

Chemical Structures for compounds denoted in the above table are provided below:

| Compound No. | Chemical Structure |
|---|---|
| A1-3 | 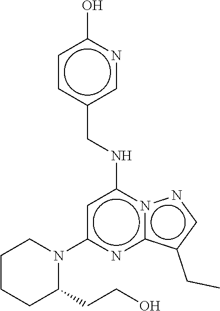 |
| A1-8 | 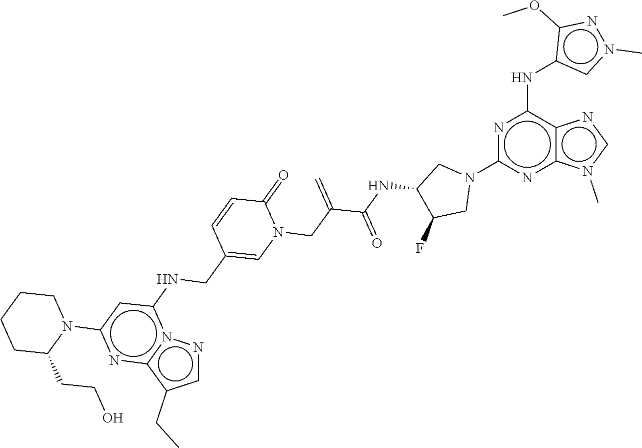 |

### Example 22—GSPT1 HiBit Degradation Assay

Exemplary compounds were tested for ability to cause degradation of GSPT1 according to experimental procedures provided below.

Promega developed a HEK293 cell line that constitutively expresses LgBiT protein and GSPT1_HiBiT fusion protein. When both proteins are present, the HiBiT portion of the fusion protein combines with LgBiT protein to form a functional luciferase enzyme. Endurazine, a cell permeable small molecule, is converted by cellular esterases into furimazine, which the luciferase enzyme uses as a substrate to generate luminescence. When GSPT1 is degraded, the HiBit is also degraded. This prevents formation of fully functional luciferase, leading to a loss in luminescence. GSPT1 degradation therefore correlates with a decrease in luminescence and can be measured continuously over time. The concentration of compound at which 50% GSPT1 degradation occurs (DC₅₀) may be calculated using this system.

### Protocol:

HEK293 LgBiTGSPT1 HiBiT CRISPR Knock-in cells were cultured in DMEM Media supplemented with 10% Heat-inactivated FBS, 1% Penicillin/Streptomycin, 200 μg/mL hygromycin and stored in a humidified incubator set at 37° C. and 5% CO₂. 18-24 hours prior to compound treatment, cells were seeded at 1000 cells/well in a 384-well tissue culture-treated white plates in 25 μL media lacking selection for 18-24 hours. The plates were spun at 300 g for 30 seconds and stored in the incubator overnight. After 24 hours, compounds are titrated in DMSO and added to media supplemented with endurazine substrate. A 25 μL aliquot of 2× compound/endurazine in media is then added to the 384-well microplate with seeded cells. (Endurazine is used at a final concentration of 0.5×). The plate was spun at 300 g for 30 seconds and stored in the incubator. After 6 hours, the plate was read on a Perkin Elmer MultiMode Plate Reader Envision 2105 to measure luminescence signal. Data were plotted in Prism (Graphpad) and the concentration of compound at which 50% GSPT1 degradation occurs (DC₅₀) was calculated.

### Part II—Results

Results showing ability of exemplary compounds to degrade GSPT1 are provided in Table 14 below. The symbol “++++” indicates a DC₅₀ less than 0.05 μM. The symbol “+++” indicates an DC₅₀ in the range of 0.05 μM to 0.5 μM. The symbol “++” indicates a DC₅₀ in the range of greater than 0.5 μM to 2.5 μM. The symbol “+” indicates a DC₅₀ greater than 2.5 mM. The symbol “N/A” indicates that no data was available.

TABLE 14

| Compound No. | DC₅₀ |
|---|---|
| I-1 | ++ |
| I-2 | +++ |
| I-3 | + |
| I-30 | ++ |
| I-31 | + |
| I-32 | +++ |
| I-114 | ++ |
| I-33 | + |
| I-34 | +++ |
| I-35 | + |
| I-36 | + |
| I-37 | + |
| I-38 | + |
| I-39 | + |
| I-40 | + |
| I-41 | + |
| I-42 | + |
| I-43 | +++ |
| I-44 | + |
| I-115 | ++++ |
| I-45 | ++ |
| I-46 | ++ |
| I-47 | +++ |
| I-48 | ++ |
| I-49 | ++ |
| I-50 | + |
| I-51 | + |
| I-52 | ++ |
| I-53 | +++ |
| I-54 | ++++ |
| I-116 | ++ |
| I-55 | +++ |
| I-56 | + |
| I-57 | +++ |
| I-58 | + |
| I-59 | +++ |
| I-60 | ++ |
| I-61 | +++ |
| I-62 | ++ |
| I-63 | + |
| I-64 | ++ |
| I-65 | + |
| I-66 | +++ |
| I-67 | ++ |
| I-68 | + |
| I-69 | +++ |
| I-70 | + |
| I-71 | ++ |
| I-72 | + |
| I-73 | +++ |
| I-74 | ++ |
| I-75 | + |
| I-76 | +++ |
| I-77 | +++ |
| I-78 | ++ |
| I-79 | ++ |
| I-80 | + |
| I-81 | +++ |
| I-82 | + |
| I-83 | ++ |
| I-84 | +++ |
| I-85 | +++ |
| I-86 | ++++ |
| I-87 | +++ |
| I-88 | ++++ |
| I-89 | ++ |
| I-90 | +++ |

### Example 23—CCNK HiBiT Degradation Assay

Exemplary compounds were tested for ability to cause degradation of CCNK. Experimental procedures and results are provided below.

### Part I—Experimental Procedure

HEK293 LgBiT cells (Promega) were cultured in DMEM (Gibco 11995), supplemented with 10% Heat-inactivated FBS (Gibco A38400-01) and 1% Penicillin/Streptomycin (Gibco 15140-122) and 200 μg/ml Hygromycin B (Gibco 10-687-010) at 37° C. with 5% CO₂ in a humidified tissue culture incubator. Cells were made stable for CCNK fused to a C-terminal HiBiT peptide by lentiviral transduction, followed by selection with 1 μg/ml Puromycin Dihydrochloride (Thermo A1113803). HEK293 LgBiT/CCNK HiBiT cells were seeded at 3000 cells/well in 384-well, tissue culture-treated white plates (Perkin Elmer 6007680) in 25 μL media lacking selection for 18-24 hours. Plates were spun at 300 g for 30 seconds and stored in the incubator overnight. After 24 hours, compounds were titrated in DMSO and added to media supplemented with Endurazine Substrate (Promega N2570). A 25 ul aliquot of 2× compound/endurazine in media were then added to the 384-well microplate with seeded cells. (Endurazine was used at a final concentration of 0.5×). The plate was spun at 300 g for 30 seconds and stored in the incubator. After 6 hours the plate was read on Envision MultiMode Plate Reader 2105 (Perkin Elmer) to measure luminescence signal. Data were plotted in Prism (Graphpad) and the concentration of compound at which 50% CCNK degradation occurs (DC₅₀) was calculated.

### Part II—Results

Experimental results showing CCNK degradation observed in the assay are provided in Table 15 below. The symbol “++++” indicates a DC₅₀ less than 0.25 μM. The symbol “+++” indicates a DC₅₀ in the range of 0.25 μM to 5 μM. The symbol “++” indicates a DC₅₀ in the range of greater than 5 μM to 10 μM. The symbol “+” indicates a DC₅₀ of greater than 10 M. The symbol “N/A” indicates that no data was available.

TABLE 15

| Compound No. | DC₅₀ |
|---|---|
| II-184 | ++++ |
| II-185 | ++++ |
| V-4 | ++++ |
| II-186 | +++ |
| II-187 | ++++ |
| II-188 | ++++ |
| II-189 | ++++ |
| II-190 | ++++ |

### Example 24—KRAS G12C Cellular Target Engagement Assay

Exemplary compounds were tested for ability to engage cellular KRAS G12C in cells. Experimental procedures and results are provided below.

### Part I—Experimental Procedure

SW1573 cells (ATCC CRL-2170) were cultured in RPMI 1640 (Gibco A1049101), supplemented with 10% Heat-inactivated FBS (Gibco Cat. #A38400-01) and 1% Penicillin/Streptomycin (Gibco Cat. #15140-122) in an incubator set at 37° C. and 5% CO2. Cells were seeded at 100,000 cells/ml in 1 ml of complete media in a 24-well, tissue culture-treated plate (Falcon 353226) and incubated overnight. Cells were treated with increasing concentrations of compound and let incubate at 37C and 5% CO2 for 17 hours, after which media was aspirated and cells were lysed in 75 uL RIPA Lysis and Extraction Buffer (Thermo 89901) supplemented with 5 mM MgCl₂, Protease and Phosphatase inhibitor cocktail (Thermo 1861281), and Universal Nuclease (Thermo 88700). Plates with RIPA were shaken at 4° C. at 600 rpm for 15 min. After shaking samples, they were collected and spun down at 21,000 g for 15 min at 4° C. Following centrifugation, ˜10 ug of lysate was added to Laemmli Sample Buffer (Bio-Rad 1610747) plus 10% beta-mercaptoethanol, boiled for 15 minutes at 65° C. and loaded onto 12% Mini-PROTEAN TGX™ Precast Protein Gels (Bio-Rad). Protein was transferred to a nitrocellulose membrane (Thermo LC2000) using the iBlot 2 Dry Blotting System (Thermo). After transfer, membranes were washed in 1× TBST (Bioland Scientific LLC TBST0103) 3× for 5 min. Following the wash, blocking buffer (Rockland MB-070) was added for 1 hour at room temperature. Primary antibodies against KRAS (LifeSpan Bio LS-C175665) and actin (Cell Signaling Technology 8457) were added overnight at 4° C. Membranes were then washed again 3× for 5 min in 1× TBST. Secondary antibodies (LI-COR 926-68072 and 926-68073) were added and shaken for 1 hr at room temperature. Membranes were then washed 3× for 5 min in 1×TBST and developed. Membranes were placed on an Odyssey imager (LI-COR).

### Part II—Results

Experimental results showing TE₅₀ observed in the assay are provided in Table 16 below. The symbol “++++” indicates a TE₅₀ less than 0.25 μM. The symbol “+++” indicates a TE₅₀ in the range of 0.25 μM to 5 μM. The symbol “++” indicates a TE₅₀ in the range of greater than 5 μM to 10 μM. The symbol “+” indicates a TE₅₀ of greater than 10 μM. The symbol “N/A” indicates that no data was available.

TABLE 16

| Compound No. | TE₅₀ |
|---|---|
| I-35 | +++ |
| I-44 | +++ |
| I-46 | +++ |
| I-48 | +++ |
| I-49 | ++++ |
| I-50 | +++ |
| I-52 | +++ |
| I-116 | ++++ |
| I-56 | ++ |
| I-58 | ++ |
| I-62 | +++ |
| I-63 | +++ |
| I-64 | +++ |
| I-65 | +++ |
| I-66 | +++ |
| I-67 | ++ |
| I-68 | +++ |
| I-72 | ++ |
| I-73 | ++++ |
| I-74 | ++++ |
| I-75 | ++ |
| I-76 | +++ |
| I-77 | +++ |
| I-78 | ++++ |
| I-79 | ++++ |
| I-80 | ++++ |
| I-84 | + |
| I-85 | ++++ |
| I-86 | +++ |
| I-88 | ++++ |
| I-89 | +++ |
| I-90 | ++++ |
| II-186 | +++ |
| II-187 | +++ |
| II-190 | +++ |

### Example 25—EGFR Cellular Target Engagement Assay

Exemplary compounds were tested for ability to engage the EGFR T790M/L858R kinase domain in cells. Experimental procedures and results are provided below.

### Part I—Experimental Procedure

HEK293 cells were cultured in DMEM (Gibco 11995), supplemented with 10% Heat-inactivated FBS (Gibco A38400-01) and 1% Penicillin/Streptomycin (Gibco 15140-122) at 37° C. with 5% CO₂ in a humidified tissue culture incubator. Cells were made stable for EGFR (amino acids 696-1022, T790M, L858R) by lentiviral transduction, followed by selection with 1 μg/ml Puromycin Dihydrochloride (Thermo A1113803). Cells were seeded at 30,000 cells/ml in 0.1 ml of complete media in Poly-D-lysine-treated 96-well plates and incubated overnight. Cells were treated with increasing concentrations of compound and let incubate at 37C and 5% CO₂ for 6 hours, after which media was aspirated, washed with 1×PBS and cells were lysed in RIPA Lysis and Extraction Buffer (Thermo 89901) supplemented with 5 mM MgCl₂, Protease and Phosphatase inhibitor cocktail (Thermo 1861281), and Universal Nuclease (Thermo 88700). Plates with RIPA were shaken at 4° C. at 600 rpm for 15 min. After shaking samples, they were collected and spun down at 21,000 g for 15 min at 4° C. Following centrifugation, −10 ug of lysate was added to Laemmli Sample Buffer (Bio-Rad 1610747) plus 10% beta-mercaptoethanol, boiled for 15 minutes at 65° C. and loaded onto 4-20% Mini-PROTEAN TGX™ Precast Protein Gels (Bio-Rad). Protein was transferred to a nitrocellulose membrane (Thermo LC2000) using the iBlot 2 Dry Blotting System (Thermo). After transfer, membranes were washed in 1× TBST (Bioland Scientific LLC TBST0103) 3× for 5 min. Following the wash, blocking buffer (Rockland MB-070) was added for 1 hour at room temperature. Primary antibodies against EGFR (Cell Signaling Technology 3197) and actin (Cell Signaling Technology 3700S) were added overnight at 4° C. Membranes were then washed again 3× for 5 min in 1× TBST. Secondary antibodies (LI-COR 926-68072 and 926-68073) were added and shaken for 1 hr at room temperature. Membranes were then washed 3× for 5 min in 1×TBST and developed. Membranes were placed on an Odyssey imager (LI-COR).

### Part II—Results

Experimental results showing TE₅₀ observed in the assay are provided in Table 17 below. The symbol “++++” indicates a TE₅₀ less than 0.25 μM. The symbol “+++” indicates a TE₅₀ in the range of 0.25 μM to 5 μM. The symbol “++” indicates a TE₅₀ in the range of greater than 5 μM to 10 μM. The symbol “+” indicates a TE₅₀ of greater than 10 μM. The symbol “N/A” indicates that no data was available.

TABLE 17

| Compound No. | TE₅₀ |
|---|---|
| I-41 | + |
| I-42 | ++ |
| I-47 | +++ |
| I-54 | +++ |
| I-55 | + |
| I-59 | +++ |
| I-60 | ++++ |
| I-61 | + |
| I-69 | +++ |
| I-70 | + |
| I-71 | + |
| I-87 | ++++ |
| II-184 | +++ |
| II-191 | + |
| II-193 | ++++ |
| II-183 | +++ |
| II-185 | + |
| V-4 | + |
| II-188 | ++++ |
| II-189 | +++ |

### Example 26—Payload Release Assay (DC₅₀ Readout)

Exemplary compounds were tested for ability to cause degradation of CCNK or GSPT1 in the presence of recombinant target protein. Experimental procedures and results are provided below.

### Part I—Experimental Procedure

HEK293 LgBiT/GSPT1 HiBiT CRISPR Knock-in cells (Promega) were cultured in DMEM (Gibco 11995), supplemented with 10% Heat-inactivated FBS (Gibco A38400-01) and 1% Penicillin/Streptomycin (Gibco 15140-122) and 200 Vg/ml Hygromycin B (Gibco 10-687-010) at 37° C. with 5% CO₂ in a humidified tissue culture incubator. HEK293 LgBiT/CCNK HiBiT cells (in-house, described above) were cultured in DMEM (Gibco 11995), supplemented with 10% Heat-inactivated FBS (Gibco A38400-01) and 1% Penicillin/Streptomycin (Gibco 15140-122) 200 Vg/ml Hygromycin B (Gibco 10-687-010), and 1 Vg/ml Puromycin Dihydrochloride (Thermo A1113803) at 37° C. with 5% CO₂ in a humidified tissue culture incubator. Cells were seeded (1000 cells/well for HEK293 LgBiT/GSPT1 HiBiT cells or 3000 cells/well for HEK293 LgBiT/CCNK HiBiT cells) 384-well, tissue culture-treated white plates (Perkin Elmer 6007680) in 10 μL media lacking selection for 18-24 hours. Plates were spun at 300 g for 30 seconds and stored in the incubator overnight. After 24 hours, a 5 μL aliquot of recombinant target protein (final concentration of either 5 μM KRAS amino acids 1-169, C51S, C80L, C118S, G12C or 3 μM EGFR amino acids 696-1022 or 3 μM EGFR amino acids 696-1022, T790M, L858R) was added to wells, followed immediately by a 5 μL aliquot of 4× compound-containing medium supplemented with Endurazine Substrate (Promega N2570) was added in each well, at a final top concentration of 10 μM test compound and 0.5× Endurazine, with 4-fold dilutions, using DMSO alone as a negative control. Plates were then spun at 300×g for 3 minutes again and cultured at 37° C. with 5% CO₂. After 6 hours the plate was read on Envision MultiMode Plate Reader 2105 (Perkin Elmer) to measure luminescence signal. Data was normalized to DMSO treated cell wells. A four-parameter non-linear regression curve fit was applied to dose-response data in Prism to determine the half maximal growth inhibitory concentration (GI₅₀) of each compound.

### Part II—Results

Experimental results showing the DC₅₀ observed in the assay at the indicated concentration of protein are provided in Table 18 below. The symbol “++++” indicates a DC₅₀ less than 0.25 μM. The symbol “+++” indicates a DC₅₀ in the range of 0.25 μM to 5 μM. The symbol “++” indicates a DC₅₀ in the range of greater than 5 μM to 10 μM. The symbol “+” indicates a DC₅₀ of greater than 10 μM. The symbol “N/A” indicates that no data was available.

TABLE 18

| Compound | DC₅₀ at 0 μM | DC₅₀ at 5 μM |
|---|---|---|
| No. | KRAS G12C | KRAS G12C |
| I-35 | +++ | ++++ |
| I-44 | + | +++ |
| I-46 | +++ | ++++ |
| I-48 | +++ | +++ |
| I-49 | +++ | + |
| I-50 | +++ | +++ |
| I-52 | +++ | ++++ |
| I-116 | +++ | ++++ |
| I-56 | ++++ | ++++ |
| I-57 | ++++ | ++++ |
| I-58 | + | ++ |
| I-62 | +++ | ++++ |
| I-63 | + | + |
| I-64 | +++ | ++++ |
| I-65 | + | ++++ |
| I-66 | ++++ | ++++ |
| I-67 | +++ | +++ |
| I-68 | + | + |
| I-72 | + | +++ |
| I-73 | ++++ | ++++ |
| I-74 | +++ | ++++ |
| I-75 | + | + |
| I-76 | ++++ | ++++ |
| I-77 | ++++ | ++++ |
| I-78 | + | + |
| I-79 | +++ | + |
| I-80 | +++ | ++++ |
| I-81 | +++ | + |
| I-82 | + | ++ |
| I-83 | +++ | + |
| I-84 | ++++ | ++++ |
| I-85 | +++ | ++++ |
| I-86 | ++++ | ++++ |
| I-88 | ++++ | ++++ |
| I-89 | +++ | + |
| I-90 | ++++ | ++++ |
| II-186 | +++ | ++++ |
| II-187 | ++++ | ++++ |
| II-190 | ++++ | ++++ |

TABLE 19

| Compound | DC₅₀ at 0 μM EGFR | DC₅₀ at 3 μM EGFR |
|---|---|---|
| No. | T790M L858R | T790M L858R |
| I-42 | +++ | ++++ |
| I-45 | ++++ | ++++ |
| I-47 | ++++ | ++++ |
| I-54 | ++++ | ++++ |
| I-55 | ++++ | ++++ |
| I-59 | ++++ | ++++ |
| I-60 | +++ | +++ |
| I-61 | ++++ | ++++ |
| I-69 | ++++ | ++++ |
| I-70 | +++ | +++ |
| I-71 | +++ | +++ |
| I-87 | ++++ | N/A |
| II-193 | ++++ | ++++ |
| II-182 | ++ | N/A |
| II-183 | ++++ | N/A |
| II-185 | +++ | ++++ |
| V-4 | ++++ | ++++ |
| II-188 | ++++ | N/A |
| II-189 | +++ | N/A |
| II-191 | +++ | N/A |

TABLE 20

| Compound | DC₅₀ at 0 μM | DC₅₀ at 3 μM |
|---|---|---|
| No. | EGFR WT | EGFR WT |
| I-42 | ++ | ++++ |
| I-45 | ++++ | ++++ |
| I-47 | ++++ | ++++ |
| I-54 | ++++ | ++++ |
| I-55 | ++++ | +++ |
| I-59 | ++++ | ++++ |
| I-60 | +++ | +++ |
| I-61 | ++++ | ++++ |
| I-69 | ++++ | ++++ |
| I-70 | +++ | +++ |
| I-71 | +++ | +++ |
| I-87 | ++++ | N/A |
| II-193 | +++ | ++++ |
| II-182 | ++ | N/A |
| II-183 | ++++ | N/A |
| II-185 | +++ | ++++ |
| V-4 | ++++ | ++++ |
| II-188 | ++++ | + |
| II-189 | +++ | N/A |
| II-191 | ++ | N/A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 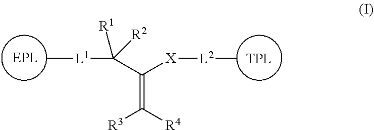
or a pharmaceutically acceptable salt thereof; wherein:
R¹, R², R³, and R⁴ are independently H, D, halo, or C₁₋₄ alkyl;
R⁵ is H or C₁₋₄ alkyl;
X is —C(O)— or —S(O)₂—;
EPL is a moiety that binds to an effector protein selected from GSPT1, Cyclin K, RBM23, RBM39, IKZF1, IKZF3, PLK1, CDK4, or CK1alpha;
TPL is a moiety that binds to a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4;
L¹ is a bond, **-linker-O—, or **-linker-N(R⁵)—, where ** is a point of attachment to EPL; and
L² is a bond or a linker;
wherein L¹ is connected to a nitrogen atom of EPL when L¹ is a bond.

2. The compound of claim 1, wherein the compound is a compound of Formula I.

3. The compound of claim 1 or 2, wherein R¹ and R² are H.

4. The compound of any one of claims 1-3, wherein R³ and R⁴ are H.

5. The compound of any one of claims 1-4, wherein X is —C(O)—.

6. The compound of any one of claims 1-4, wherein X is —S(O)₂—.

7. The compound of any one of claims 1-6, wherein the EPL is a moiety that binds to GSPT1.

8. The compound of any one of claims 1-6, wherein the EPL has the following formula: 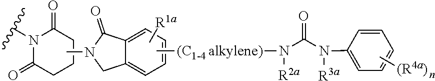
wherein:
R^{1a} is hydrogen, halo, or C₁₋₄ alkyl;
R^{2a} and R^{3A} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

9. The compound of any one of claims 1-6, wherein the EPL has the following formula: 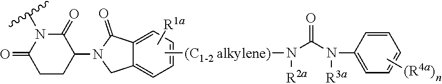
wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 1 or 2.

10. The compound of any one of claims 1-6, wherein the EPL has the following formula: 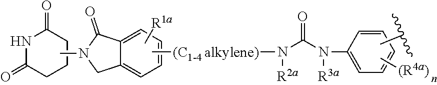
wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

11. The compound of any one of claims 1-6, wherein the EPL has the following formula: 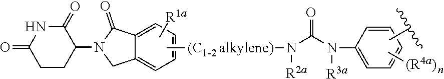
wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.

12. The compound of any one of claims 1-6, wherein the EPL is one of the following: 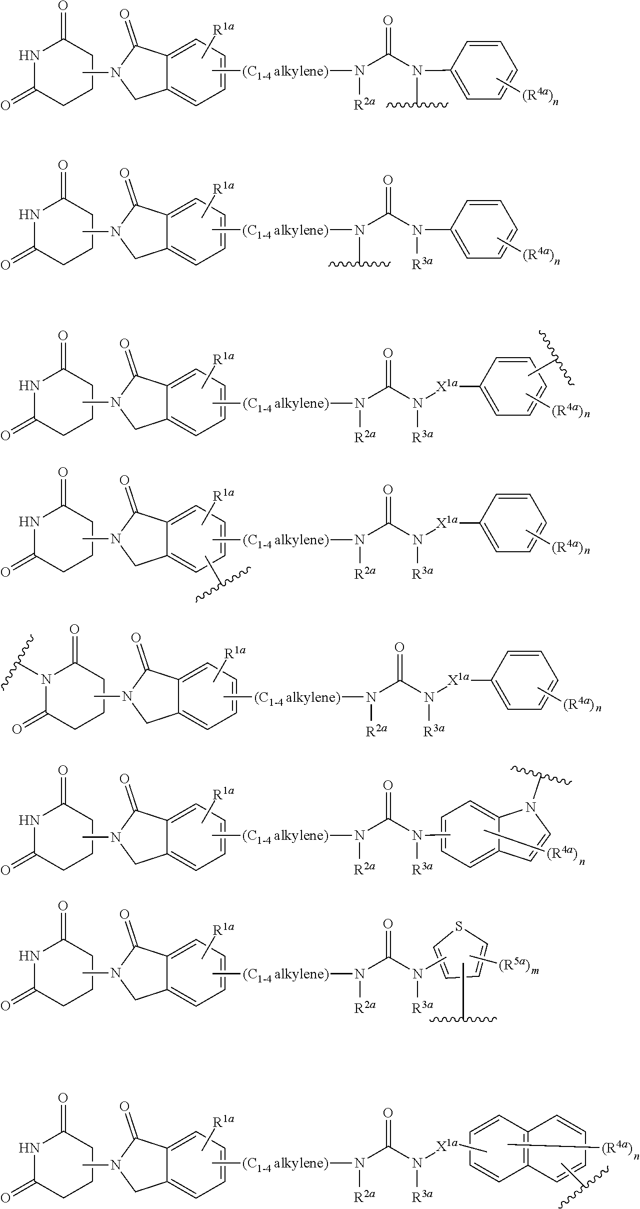
wherein:
R^{1a} is hydrogen, halo, or C₁₋₄ alkyl;
R^{2a} and R^{3a} each represent independently for each occurrence hydrogen or C₁₋₄ alkyl;
R^{4a} and R^{5a} each represent independently for each occurrence halo, C₁₋₄ alkyl, C₁₋₄ haloalkyl, hydroxyl, nitro, or C₁₋₄ alkoxyl;
X^{1a} is C₁₋₄ alkylene; and
m is 0, 1, or 2;
n is 0, 1, 2, or 3.

13. The compound of any one of claims 1-6, wherein the EPL is one of the following:

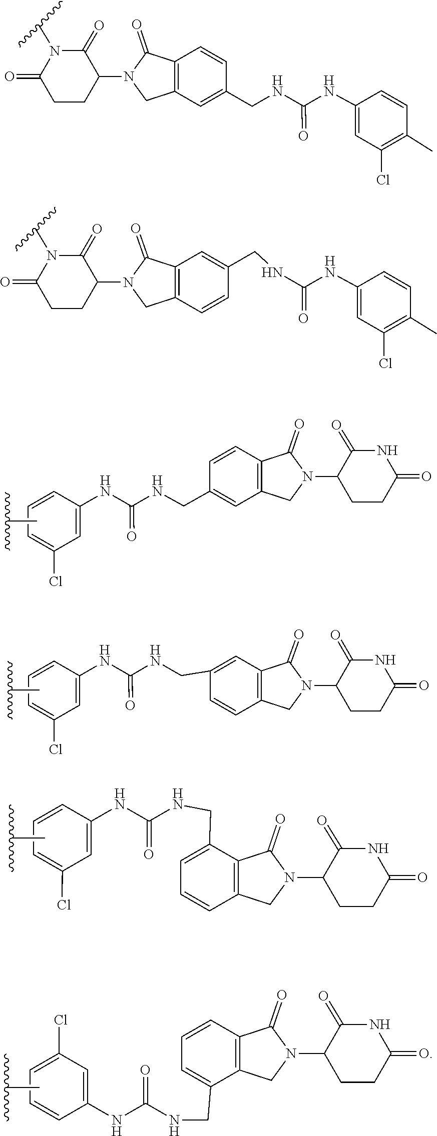

14. The compound of any one of claims 1-6, wherein the EPL is one of the following:

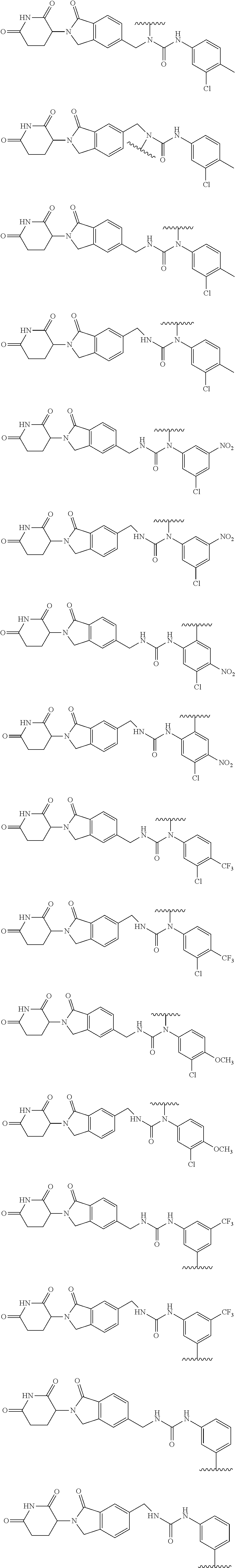

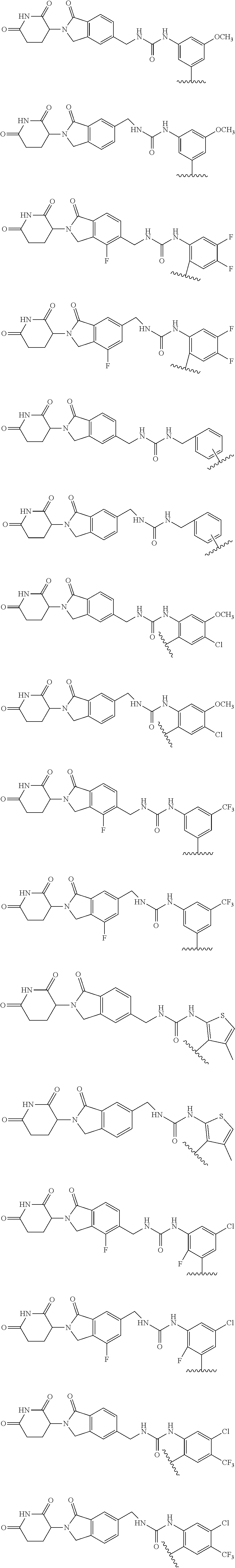

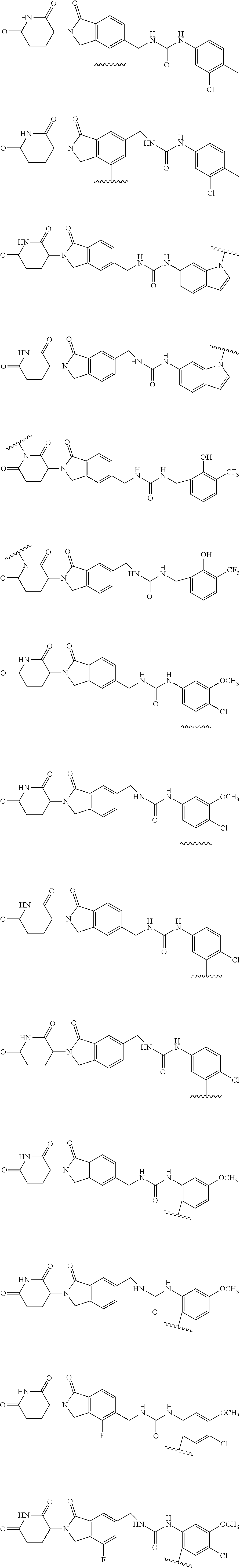

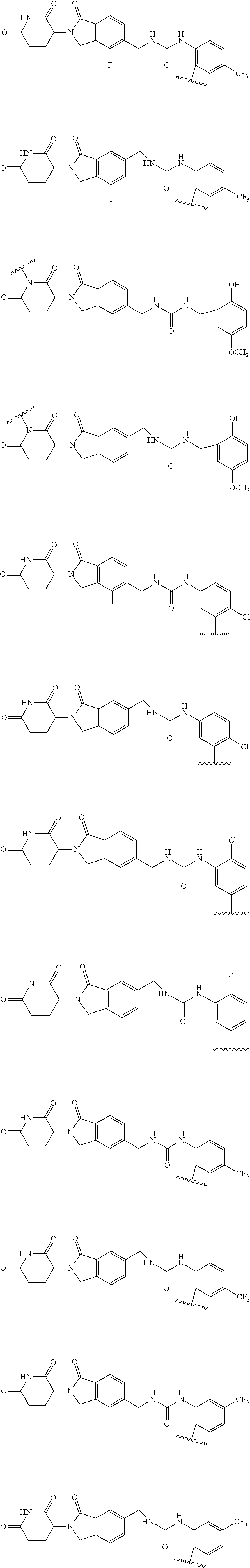

15. The compound of any one of claims 1-6, wherein the EPL is one of the following:

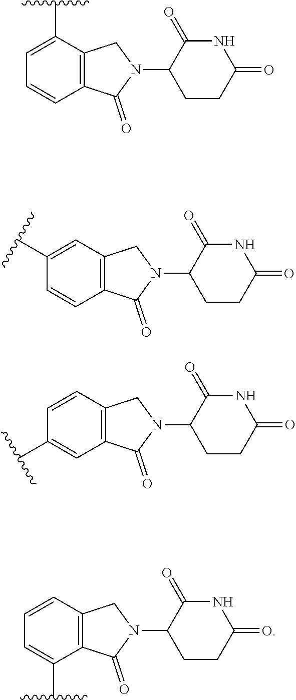 [SMILES: *c1cccc2c1CN(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *c1cccc2c1C(=O)N(C1CCC(=O)NC1=O)C2]

16. The compound of any one of claims 1-6, wherein the EPL is a moiety that binds to Cyclin K.

17. The compound of any one of claims 1-6, wherein the EPL is one of the following: 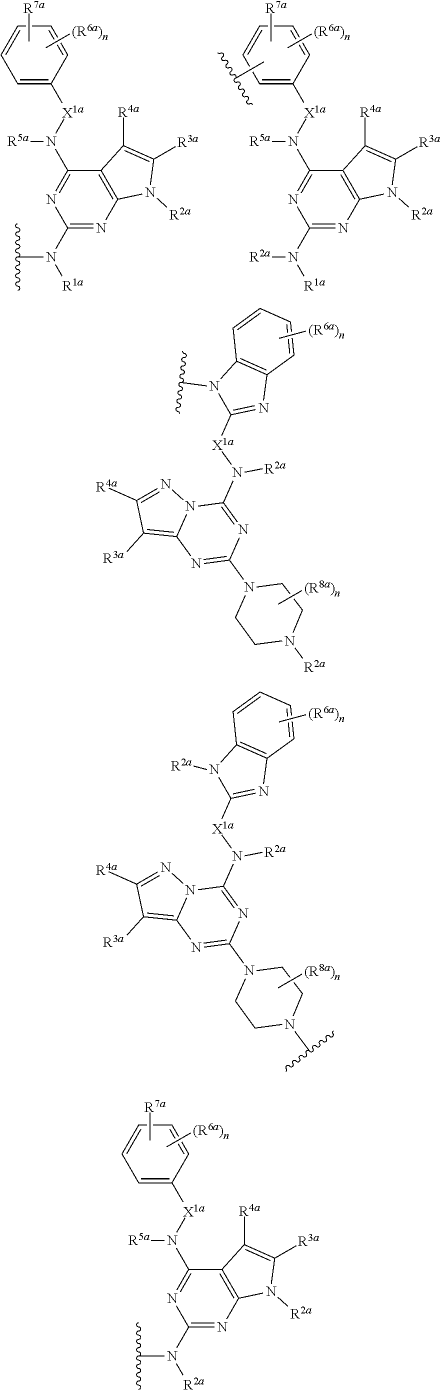
wherein:
R^{1a} is C₂₋₇ hydroxyalkyl;
R^{2a} and R^{5a} each represent independently for each occurrence hydrogen, C₁₋₄ alkyl, or C₃₋₆ cycloalkyl;
R^{3a}, R^{4a}, R^{6a}, and R^{8a} each represent independently for each occurrence hydrogen, C₁₋₄ alkyl, C₁₋₄ haloalkyl, C₃₋₆ cycloalkyl, or halo;
R^{7a} is a 5-6 membered heteroaryl containing 1, 2, or 3 heteroatoms independently selected from nitrogen, oxygen, and sulfur, wherein the heteroaryl is substituted with 0, 1, or 2 substituents independently selected from C₁₋₄ alkyl, C₁₋₄ haloalkyl, C₃₋₆ cycloalkyl, or halo;
X^{1a} is C₁₋₄ alkylene; and
n represents independently for each occurrence 0, 1, or 2.

18. The compound of any one of claims 1-6, wherein the EPL is one of the following:

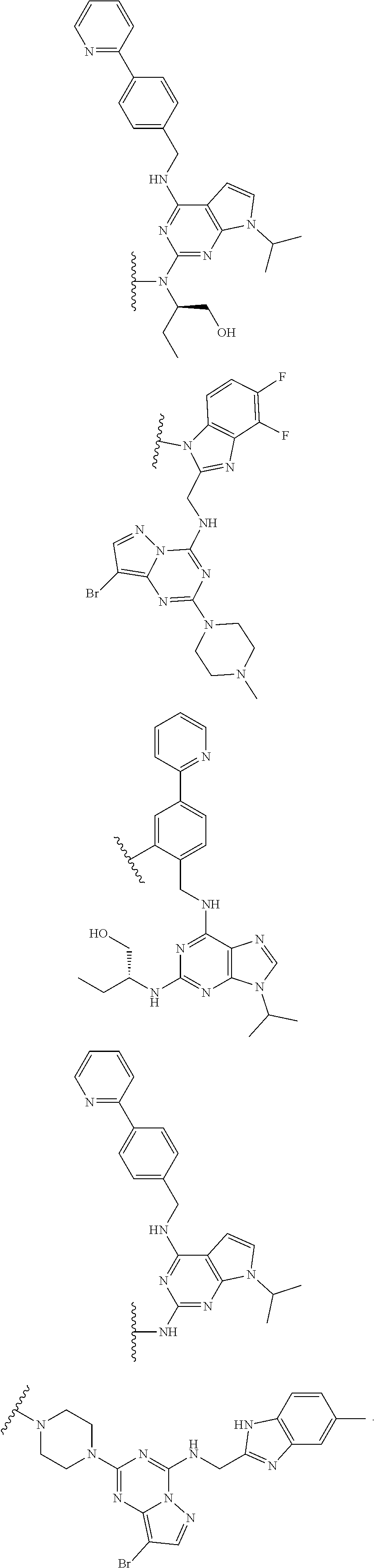

19. The compound of any one of claims 1-6, wherein the EPL is a moiety that binds to RBM23.

20. The compound of any one of claims 1-6, wherein the EPL is a moiety that binds to RBM39.

21. The compound of any one of claims 1-6, wherein the EPL is one of the following:

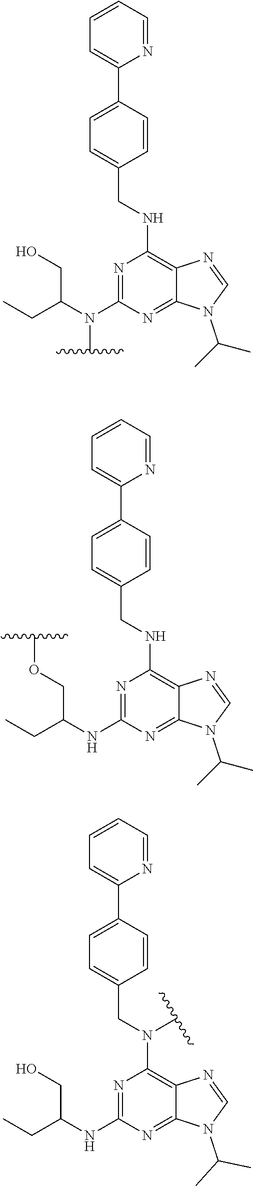

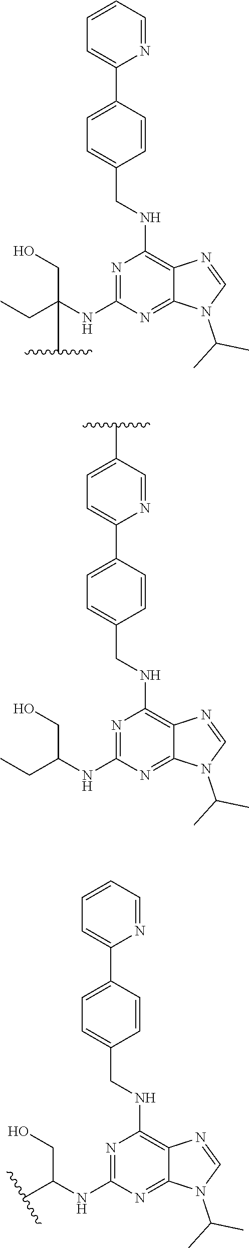

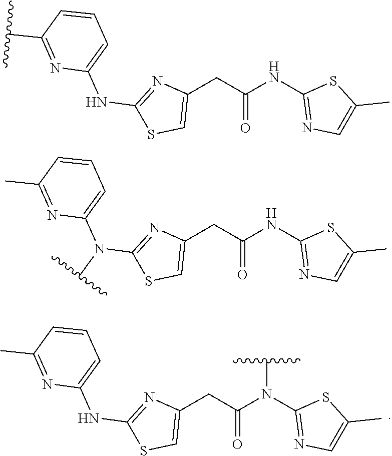

22. The compound of any one of claims 1-6, wherein the EPL is one of the following:

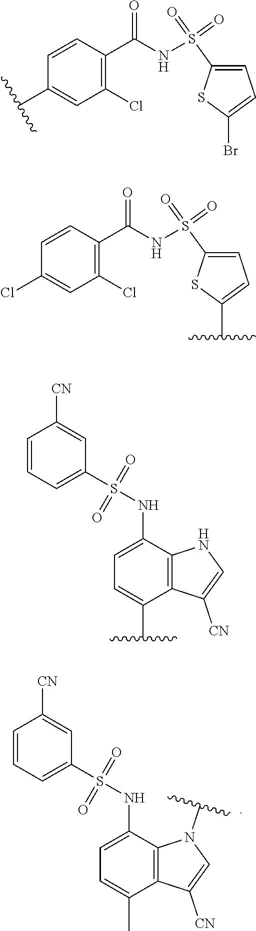 [SMILES: *c1ccc(C(=O)NS(=O)(=O)c2ccc(Br)s2)c(Cl)c1 ; *c1ccc(S(=O)(=O)NC(=O)c2ccc(Cl)cc2Cl)s1 ; *c1ccc(NS(=O)(=O)c2cccc(C#N)c2)c2[nH]cc(C#N)c12 ; *n1cc(C#N)c2c(C)ccc(NS(=O)(=O)c3cccc(C#N)c3)c21]

23. The compound of any one of claims 1-6, wherein the EPL is one of the following:

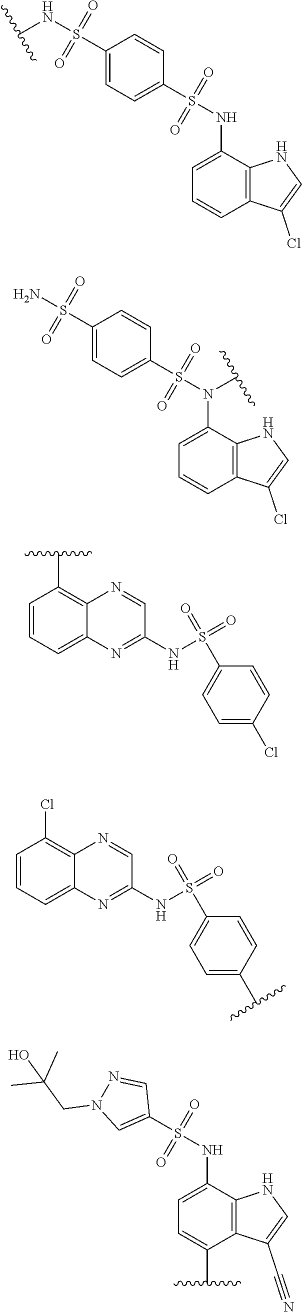 [SMILES: *NS(=O)(=O)c1ccc(S(=O)(=O)Nc2cccc3c(Cl)c[nH]c23)cc1 ; *N(c1cccc2c(Cl)c[nH]c12)S(=O)(=O)c1ccc(S(N)(=O)=O)cc1 ; *c1cccc2nc(NS(=O)(=O)c3ccc(Cl)cc3)cnc12 ; *c1ccc(S(=O)(=O)Nc2cnc3c(Cl)cccc3n2)cc1 ; *c1ccc(NS(=O)(=O)c2cnn(CC(C)(C)O)c2)c2[nH]cc(C#N)c12]

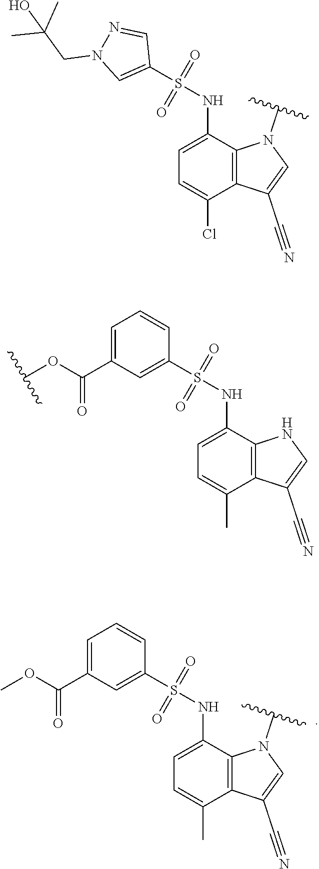 [SMILES: *n1cc(C#N)c2c(Cl)ccc(NS(=O)(=O)c3cnn(CC(C)(C)O)c3)c21 ; *OC(=O)c1cccc(S(=O)(=O)Nc2ccc(C)c3c(C#N)c[nH]c23)c1 ; *n1cc(C#N)c2c(C)ccc(NS(=O)(=O)c3cccc(C(=O)OC)c3)c21]

24. The compound of any one of claims 1-6, wherein the EPL is a moiety that binds to an effector protein selected from IKZF1 or IKZ-F3.

25. The compound of any one of claims 1-6, wherein the EPL is one of the following:

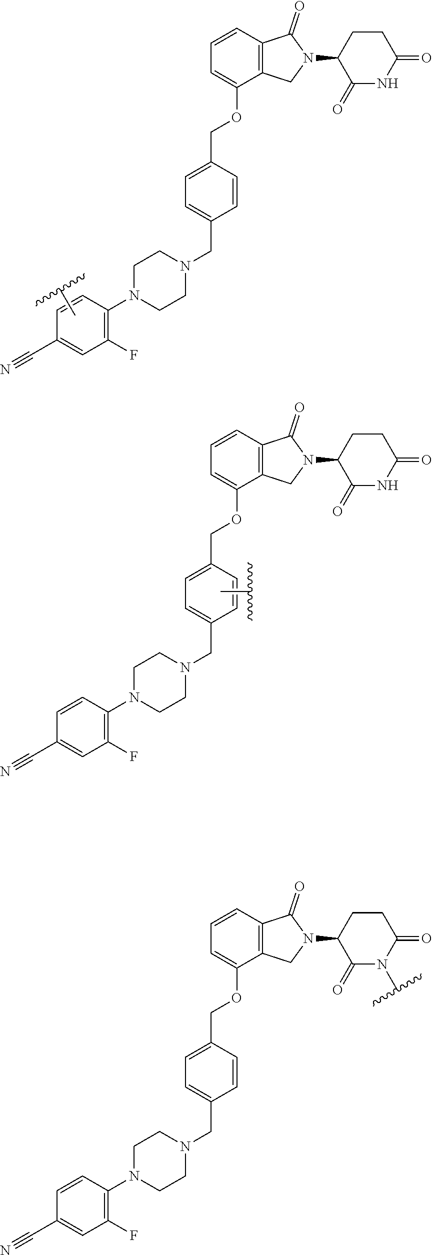

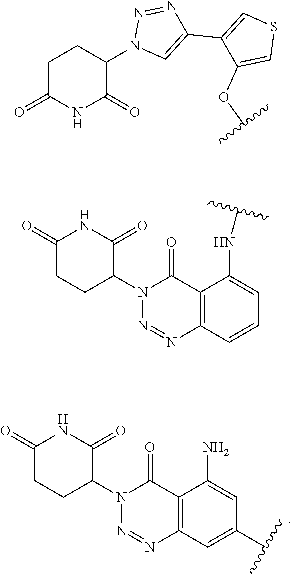 [SMILES: *Oc1cscc1-c1cn(C2CCC(=O)NC2=O)nn1 ; *Nc1cccc2nnn(C3CCC(=O)NC3=O)c(=O)c12 ; *c1cc(N)c2c(=O)n(C3CCC(=O)NC3=O)nnc2c1]

26. The compound of any one of claims 1-6, wherein the EPL is one of the following:

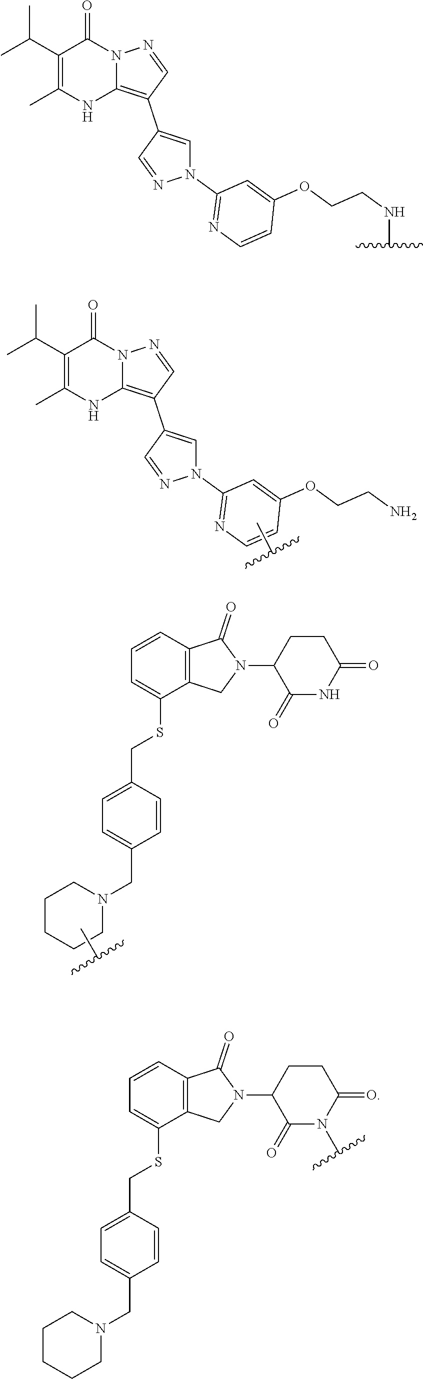

27. The compound of any one of claims 1-6, wherein the EPL is a moiety that binds to an effector protein selected from PLK1, CDK4, or CK1alpha.

28. The compound of any one of claims 1-6, wherein the EPL is one of the following:

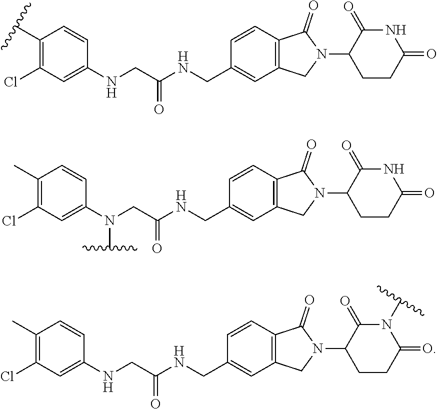 [SMILES: *c1ccc(NCC(=O)NCc2ccc3c(c2)CN(C2CCC(=O)NC2=O)C3=O)cc1Cl ; *N(CC(=O)NCc1ccc2c(c1)CN(C1CCC(=O)NC1=O)C2=O)c1ccc(C)c(Cl)c1 ; *N1C(=O)CCC(N2Cc3cc(CNC(=O)CNc4ccc(C)c(Cl)c4)ccc3C2=O)C1=O]

29. The compound of any one of claims 1-28, wherein L¹ is a bond.

30. The compound of any one of claims 1-28, wherein L¹ is **-linker-O—.

31. The compound of any one of claims 1-28, wherein L¹ is one of the following: —O—, —N(H)—, —N(C₁₋₄ alkyl)-, —OC(O)—**, —N(H)C(O)—**, —N(C₁₋₄ alkyl)C(O)—**, —O—(C₁₋₆ alkylene)-**, —N(H)—(C₁₋₆ alkylene)-**, —N(C₁₋₄ alkyl)-(C₁₋₆ alkylene)-**, —OC(O)—(C₁₋₆ alkylene)-**, —N(H)C(O)—(C₁₋₆ alkylene)-**, —N(C₁₋₄ alkyl)C(O)—(C₁₋₆ alkylene)-**, —OC(O)N(H)—(C₁₋₆ alkylene)-**, —OC(O)N(C₁₋₄ alkyl)-(C₁₋₆ alkylene)-**, —N(H)C(O)₂—(C₁₋₆ alkylene)-**, —N(C₁₋₄ alkyl)C(O)₂—(C₁₋₆ alkylene)-**, or 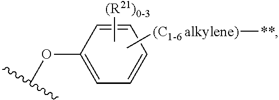
wherein R²¹ represents independently for each occurrence halo, C₁₋₄ alkyl, C₁₋₄ haloalkyl, hydroxyl, C₁₋₄ alkoxyl, or C₃₋₆ cycloalkyl.

32. The compound of any one of claims 1-28, wherein L¹ is —O—.

33. The compound of any one of claims 1-28, wherein L¹ is —OC(O)—**.

34. A compound represented by Formula I-A: 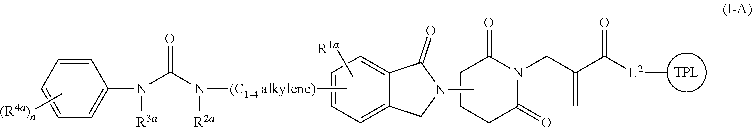
or a pharmaceutically acceptable salt thereof; wherein:
R^{1a} is hydrogen, halo, or C₁₋₄ alkyl;
R^{2a} and R^{3a} each represent independently for each occurrence hydrogen or C₁₋₄ alkyl;
R^{4a} represents independently for each occurrence halo, C₁₋₄ alkyl, C₁₋₄ haloalkyl, hydroxyl, or C₁₋₄ alkoxyl; and
n is 0, 1, 2, or 3.
TPL is a moiety that binds to a target protein selected from KRAS, HER2, BTK, EGFR, androgen receptor protein, estrogen receptor protein, ALK, IDH1, FLT3, FGFR1, FGFR4, FGFR2, FGFR3, ERK1, ERK2, FGR, HER3, or HER4;
L² is a bond or a linker.

35. The compound of claim 34, wherein R^{1a}, R^{2a}, and R^{3a} are hydrogen.

36. The compound of claim 34 or 35, wherein R^{4a} represents independently for each occurrence halo or C₁₋₄ alkyl, and n is 2.

37. The compound of any one of claims 1-36, wherein the TPL is a moiety that binds to KRAS.

38. The compound of any one of claims 1-36, wherein the TPL is one of the following: 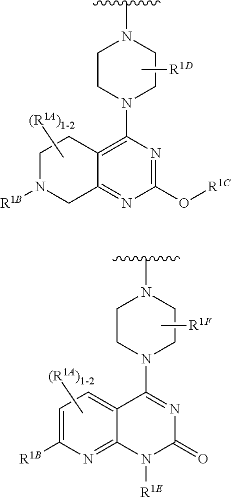
wherein:
R^{1A} represents independently for each occurrence hydrogen, halo, hydroxyl, C₁₋₄ alkyl, or C₁₋₄ alkoxyl;
R^{1B} is C₆₋₁₂ aryl or 6-12 membered heteroaryl containing 1, 2 or 3 heteroatoms selected from nitrogen, oxygen, or sulfur, wherein the aryl and heteroaryl are optionally substituted by 1, 2, or 3 substituents independently selected from halo, hydroxyl, C₁₋₄ alkyl, or C₁₋₄ alkoxyl;
R^{1C} is —(C₁₋₆ alkylene)-3-7 membered saturated mono-cyclic or bicylic heterocyclyl containing 1, 2, or 3 heteroatoms selected from nitrogen, oxygen, and sulfur;
R^{1D} is —(C₁₋₆ alkylene)-CN;
R^{1E} is C₆₋₁₂ aryl or 6-12 membered heteroaryl containing 1, 2 or 3 heteroatoms selected from nitrogen, oxygen, or sulfur, wherein the aryl and heteroaryl are optionally substituted by 1, 2, or 3 substituents independently selected from halo, hydroxyl, C₁₋₄ alkyl, or C₁₋₄ alkoxyl; and
R^{1F} is C₁₋₆ alkyl.

39. The compound of any one of claims 1-36, wherein the TPL is the following:

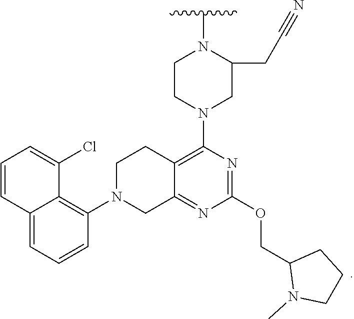 [SMILES: *N1CCN(c2nc(OCC3CCCN3C)nc3c2CCN(c2cccc4cccc(Cl)c24)C3)CC1CC#N]

40. The compound of any one of claims 1-36, wherein the TPL is the following:

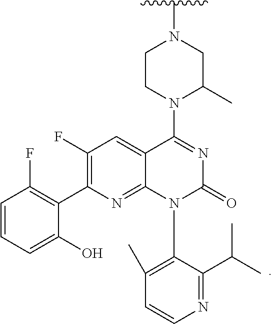 [SMILES: *N1CCN(c2nc(=O)n(-c3c(C)ccnc3C(C)C)c3nc(-c4c(O)cccc4F)c(F)cc23)C(C)C1]

41. The compound of any one of claims 1-36, wherein the TPL is one of the following:

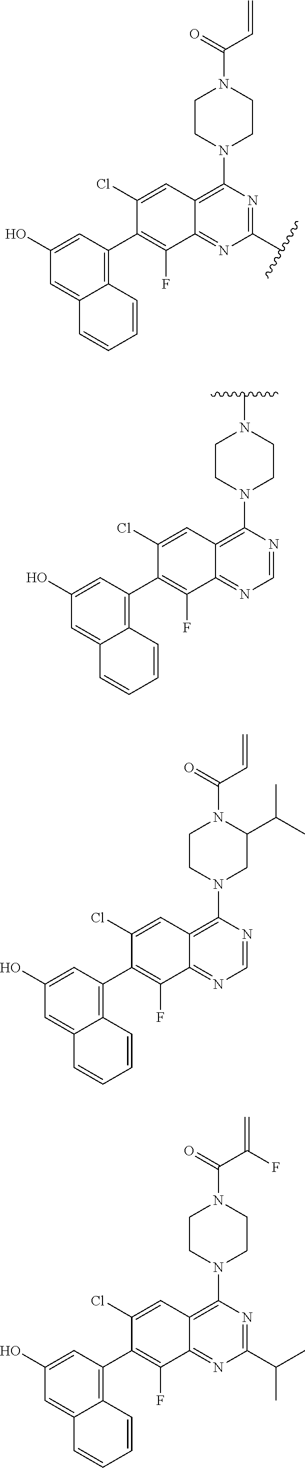 [SMILES: *c1nc(N2CCN(C(=O)C=C)CC2)c2cc(Cl)c(-c3cc(O)cc4ccccc34)c(F)c2n1 ; *N1CCN(c2ncnc3c(F)c(-c4cc(O)cc5ccccc45)c(Cl)cc23)CC1 ; C=CC(=O)N1CCN(c2ncnc3c(F)c(-c4cc(O)cc5ccccc45)c(Cl)cc23)CC1C(C)C ; C=C(F)C(=O)N1CCN(c2nc(C(C)C)nc3c(F)c(-c4cc(O)cc5ccccc45)c(Cl)cc23)CC1]

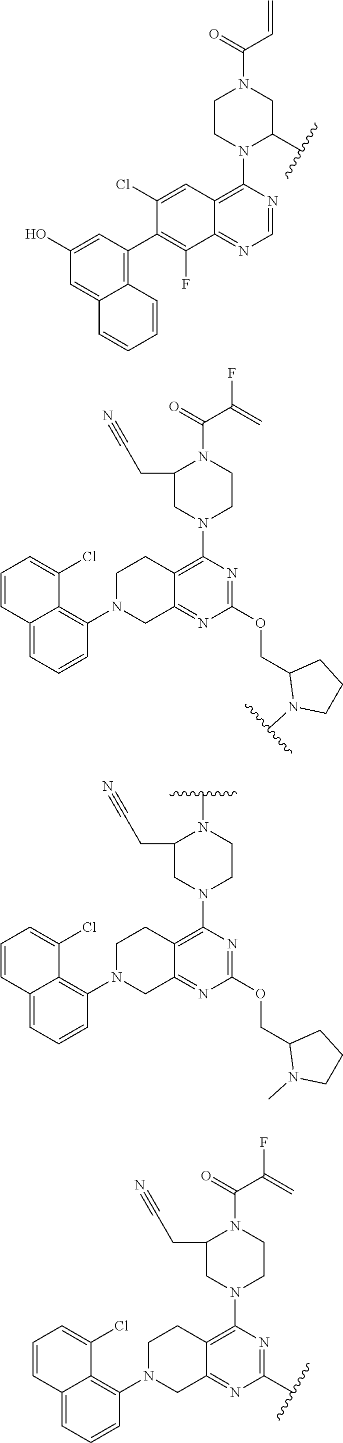

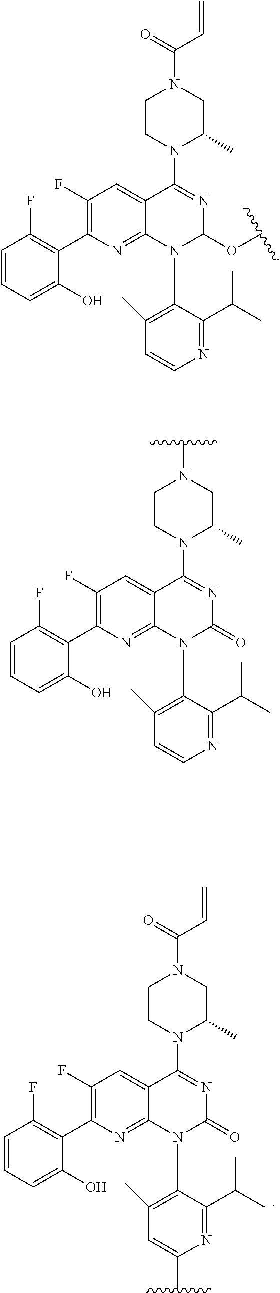 [SMILES: *OC1N=C(N2CCN(C(=O)C=C)C[C@@H]2C)c2cc(F)c(-c3c(O)cccc3F)nc2N1c1c(C)ccnc1C(C)C ; *N1CCN(c2nc(=O)n(-c3c(C)ccnc3C(C)C)c3nc(-c4c(O)cccc4F)c(F)cc23)[C@@H](C)C1 ; *c1cc(C)c(-n2c(=O)nc(N3CCN(C(=O)C=C)C[C@@H]3C)c3cc(F)c(-c4c(O)cccc4F)nc32)c(C(C)C)n1]

42. The compound of any one of claims 1-36, wherein the TPL is one of the following: 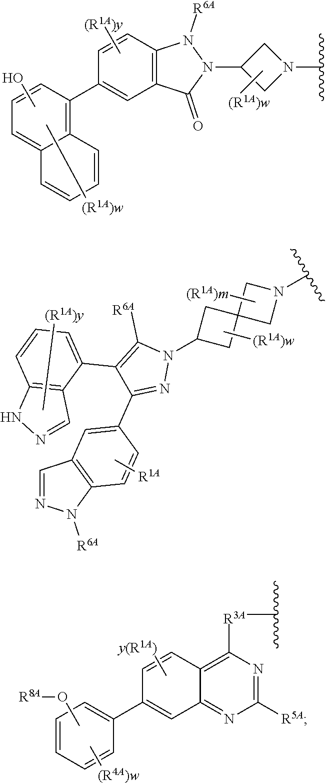
wherein:
R^{1A} and R^{4A} each represent independently for each occurrence hydrogen, halo, hydroxyl, C₁₋₄ alkyl, C₁₋₄ alkoxyl, or C₃₋₆ cycloalkyl;
R^{2A} represents independently for each occurrence hydrogen or C₁₋₄ alkyl;
R^{3A} is a 3-7 membered saturated heterocyclylene containing 1, 2, or 3 heteroatoms independently selected from oxygen, nitrogen, and sulfur, wherein the heterocyclylene is substituted with 0, 1, 2, or 3 substituents independently selected from halo and C₁₋₄ alkyl;
R^{5A} is hydrogen, halo, hydroxyl, or C₁₋₄ alkyl;
R^{6A} is C₁₋₆ alkyl or C₃₋₆ cycloalkyl;
R^{7A} is C₁₋₆ alkylene)-N(R^{8A})₂;
R^{8A} is hydrogen, C₁₋₆ alkyl, or C₃₋₆ cycloalkyl;
y and w each represent independently for each occurrence 1 or 2.

43. The compound of any one of claims 1-36, wherein the TPL is one of the following:

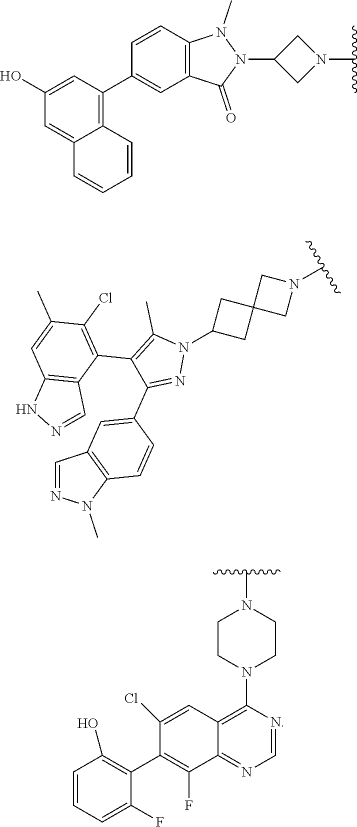 [SMILES: *N1CC(n2c(=O)c3cc(-c4cc(O)cc5ccccc45)ccc3n2C)C1 ; *N1CC2(CC(n3nc(-c4ccc5c(cnn5C)c4)c(-c4c(Cl)c(C)cc5[nH]ncc45)c3C)C2)C1 ; *N1CCN(c2ncnc3c(F)c(-c4c(O)cccc4F)c(Cl)cc23)CC1]

44. The compound of any one of claims 1-36, wherein the TPL is a moiety that binds to HER2.

45. The compound of any one of claims 1-36, wherein the TPL is one of the following 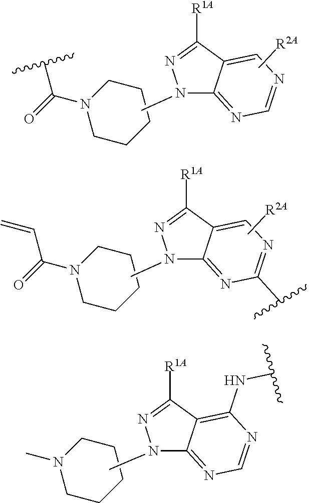
wherein:
R^{1A} is —C(O)(NR^{5A})-(phenyl optionally substituted with 1, 2, 3, or 5 substituents independently selected from halo, hydroxyl, C₁₋₄ alkyl, C₁₋₄ alkoxyl, and —(C₁₋₄ alkylene-C(O)N(R^{5A})(R^{6A}));
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

46. The compound of any one of claims 1-36, wherein the TPL is one of the following:

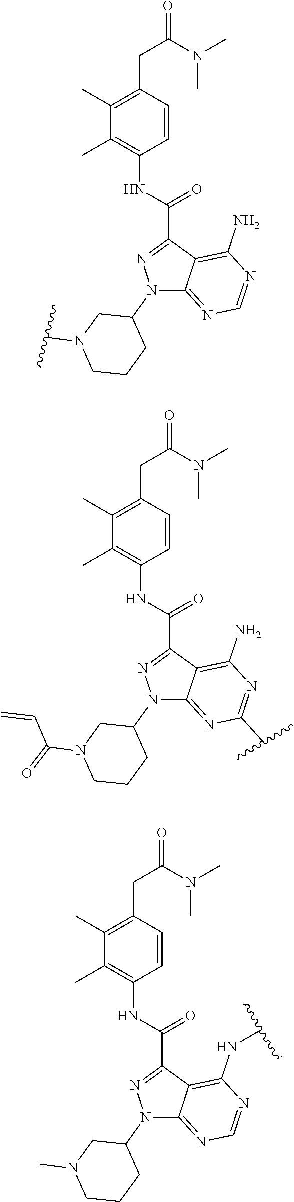 [SMILES: *N1CCCC(n2nc(C(=O)Nc3ccc(CC(=O)N(C)C)c(C)c3C)c3c(N)ncnc32)C1 ; *c1nc(N)c2c(C(=O)Nc3ccc(CC(=O)N(C)C)c(C)c3C)nn(C3CCCN(C(=O)C=C)C3)c2n1 ; *Nc1ncnc2c1c(C(=O)Nc1ccc(CC(=O)N(C)C)c(C)c1C)nn2C1CCCN(C)C1]

47. The compound of any one of claims 1-36, wherein the TPL is a moiety that binds to BTK.

48. The compound of any one of claims 1-36, wherein the TPL is one of the following: 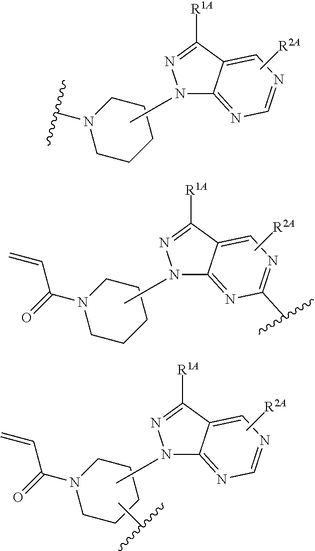
wherein:
R^{1A} is -(phenyl optionally substituted with 1, 2, 3, or 5 substituents independently selected from halo, hydroxyl, C₁₋₄ alkyl, and C₁₋₄ alkoxyl)-O-(phenyl optionally substituted with 1, 2, 3, or 5 substituents independently selected from halo, hydroxyl, C₁₋₄ alkyl, and C₁₋₄ alkoxyl);
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

49. The compound of any one of claims 1-36, wherein the TPL is the following:

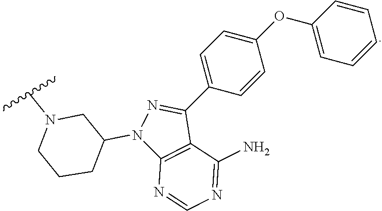 [SMILES: *N1CCCC(n2nc(-c3ccc(Oc4ccccc4)cc3)c3c(N)ncnc32)C1]

50. The compound of any one of claims 1-36, wherein the TPL is one of the following:

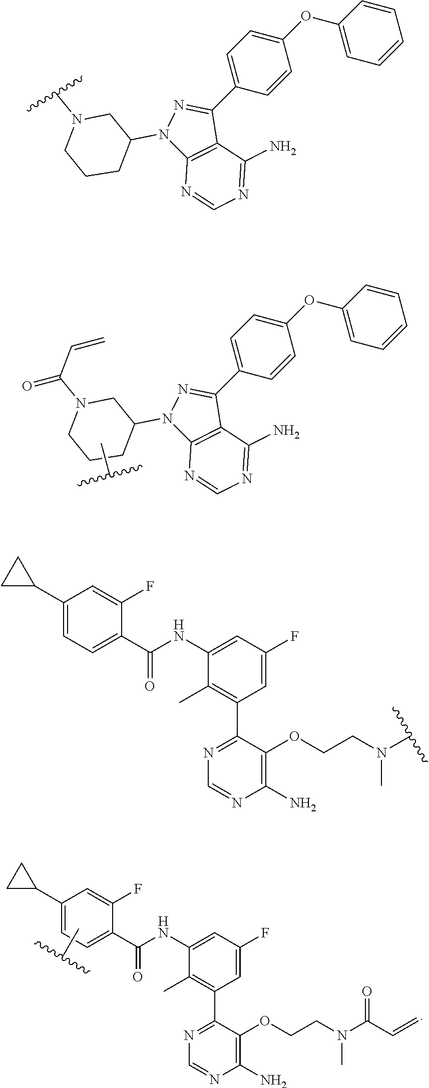

51. The compound of any one of claims 1-36, wherein the TPL is a moiety that binds to EGFR.

52. The compound of any one of claims 1-36, wherein the TPL is one of the following: 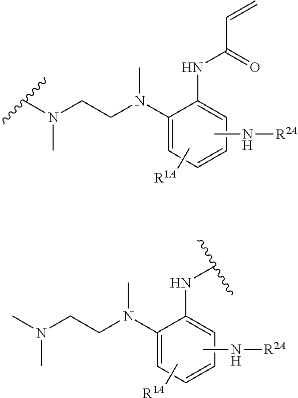
wherein:
R^{1A} is hydrogen, halo, hydroxyl, C₁₋₄ alkyl, C₁₋₄ alkoxyl, or N(R^{5A})(R^{6A}); and
R^{2A} is -(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl)-(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

53. The compound of any one of claims 1-36, wherein the TPL is one of the following: 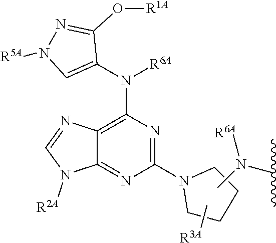
wherein:
R^{1A} is C₁₋₄ alkyl;
R^{2A} and R^{6A} are independently hydrogen or C₁₋₄ alkyl;
R^{3A} is halo;
R^{5A} is C₁₋₆ alkyl or C₃₋₆ cycloalkyl.

54. The compound of any one of claims 1-36, wherein the TPL is one of the following: 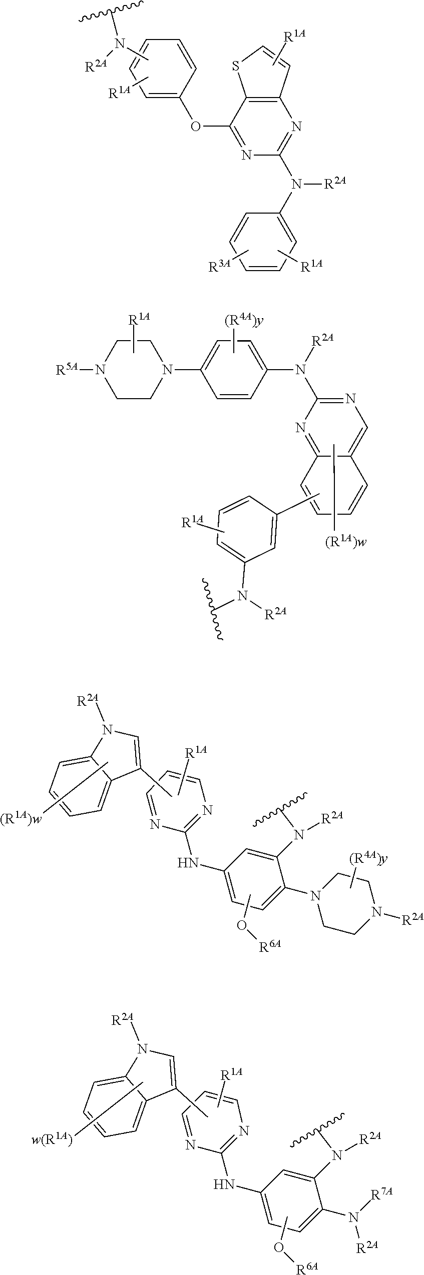
wherein:
R^{1A} and R^{4A} each represent independently for each occurrence hydrogen, halo, hydroxyl, C₁₋₄ alkyl, C₁₋₄ alkoxyl, or C₃₋₆ cycloalkyl;
R^{2A} represents independently for each occurrence hydrogen or C₁₋₄ alkyl;
R^{3A} is a 3-7 membered saturated heterocyclyl containing 1, 2, or 3 heteroatoms independently selected from oxygen, nitrogen, and sulfur, wherein the heterocyclyl is substituted with 0, 1, 2, or 3 substituents independently selected from halo, hydroxyl, C₁₋₄ alkyl, C₁₋₄ alkoxyl, or C₃₋₆ cycloalkyl;
R^{5A} is C₁₋₆ hydroxyalkyl or C₁₋₆ alkyl;
R^{6A} is C₁₋₆ alkyl or C₃₋₆ cycloalkyl;
R^{7A} is C₁₋₆ alkylene)-N(R^{2A})₂;
y and w are independently 1 or 2.

55. The compound of claim 54, wherein R^{3A} is piperazinyl substituted with 0, 1, 2, or 3 substituents independently selected from halo and C₁₋₄ alkyl.

56. The compound of any one of claims 1-36, wherein the TPL is one of the following:

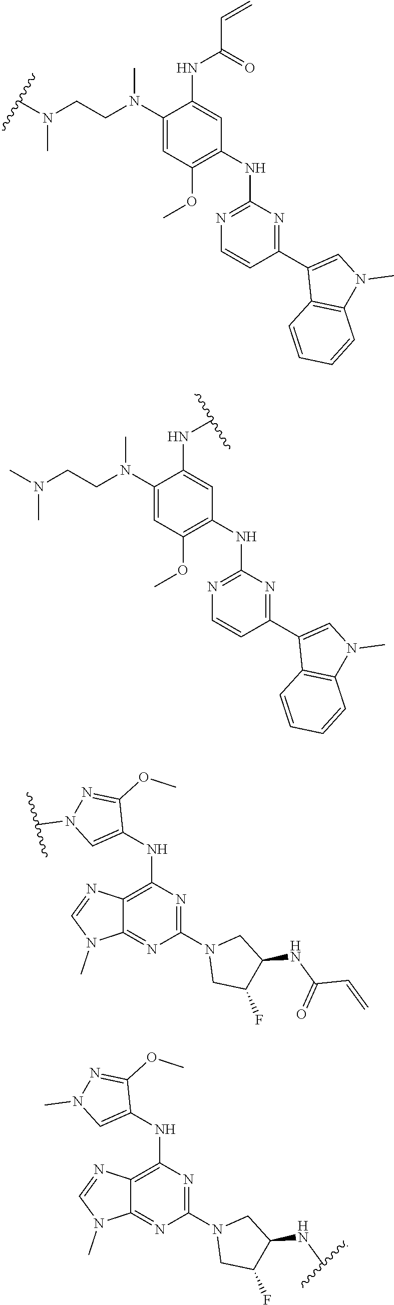

57. The compound of any one of claims 1-36, wherein the TPL is one of the following:

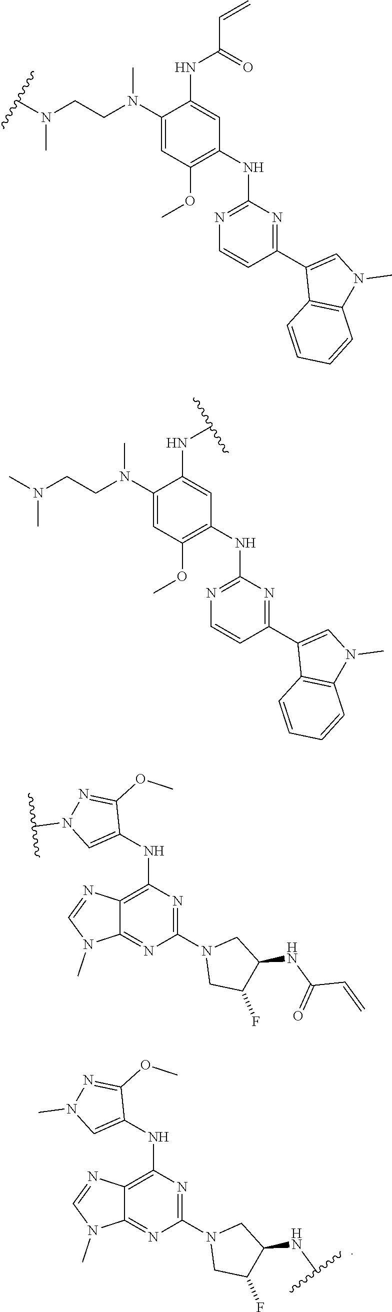

58. The compound of any one of claims 1-36, wherein the TPL is one of the following:

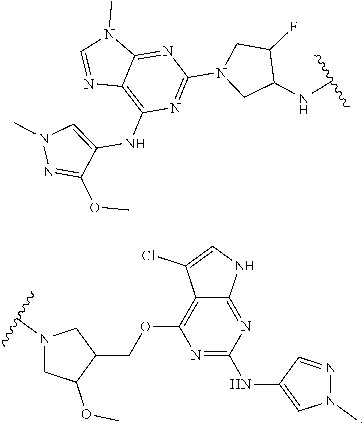 [SMILES: *NC1CN(c2nc(Nc3cn(C)nc3OC)c3ncn(C)c3n2)CC1F ; *N1CC(COc2nc(Nc3cnn(C)c3)nc3[nH]cc(Cl)c23)C(OC)C1]

59. The compound of any one of claims 1-36, wherein the TPL is one of the following:

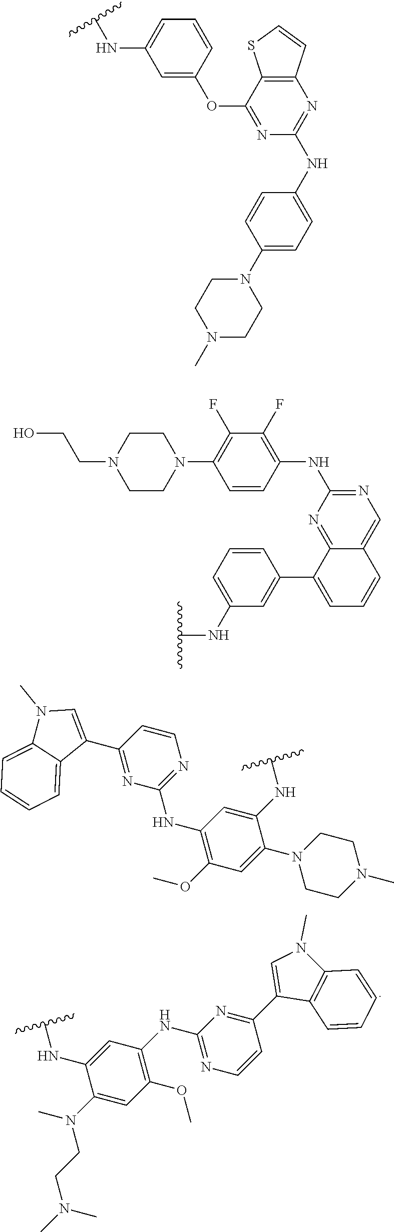

60. The compound of any one of claims 1-36, wherein the TPL is a moiety that binds to androgen receptor protein.

61. The compound of any one of claims 1-36, wherein the TPL is one of the following: 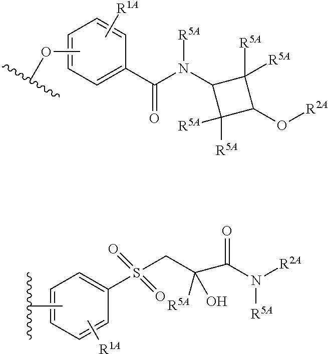
wherein:
R^{1A} is hydrogen, halo, hydroxyl, C₁₋₄ alkyl, C₁₋₄ alkoxyl, or —N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

62. The compound of any one of claims 1-36, wherein the TPL is one following:

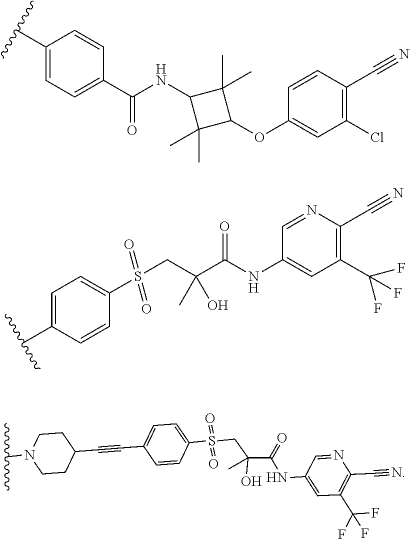 [SMILES: *c1ccc(C(=O)NC2C(C)(C)C(Oc3ccc(C#N)c(Cl)c3)C2(C)C)cc1 ; *c1ccc(S(=O)(=O)CC(C)(O)C(=O)Nc2cnc(C#N)c(C(F)(F)F)c2)cc1 ; *N1CCC(C#Cc2ccc(S(=O)(=O)CC(C)(O)C(=O)Nc3cnc(C#N)c(C(F)(F)F)c3)cc2)CC1]

63. The compound of any one of claims 1-36, wherein the TPL is a moiety that binds to a target protein selected from estrogen receptor protein or ALK.

64. The compound of any one of claims 1-36, wherein the TPL is one of the following: 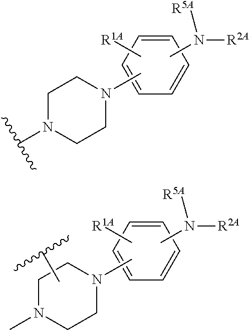
wherein:
R^{1A} is hydrogen, halo, hydroxyl, C₁₋₄ alkyl, C₁₋₄ alkoxyl, or N(RS^{A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl)-N(R^{5A})-(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄haloalkyl, C₁₋₄ alkoxyl, and —P(O)(C₁₋₆ alkyl)₂; and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

65. The compound of any one of claims 1-36, wherein the TPL is one of the following:

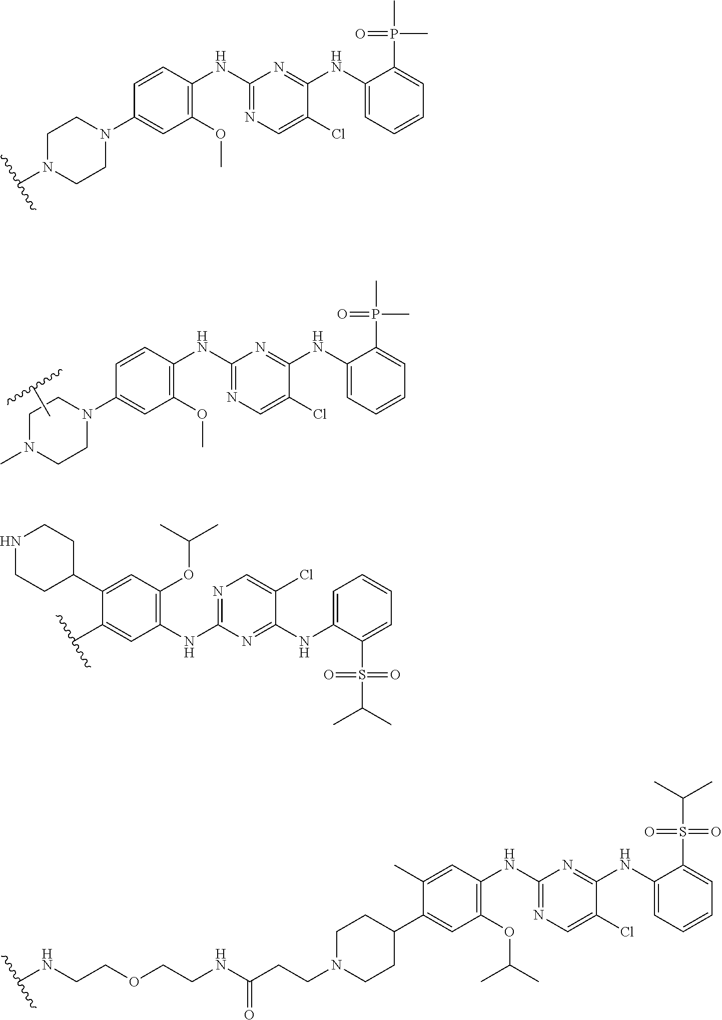

66. The compound of any one of claims 1-36, wherein the TPL is a moiety that binds to a target protein selected from IDH1, FLT3, FGFR1, FGFR4, FGFR2, FGFR3, ERK1, ERK2, FGR, HER3, or HER4.

67. The compound of any one of claims 1-66, wherein L² is a bond.

68. The compound of any one of claims 1-66, wherein L² is a linker.

69. The compound of any one of claims 1-68, wherein the linker is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

70. The compound of any one of claims 1-68, wherein the linker has the formula —(Co-12 alkylene)-(optionally substituted 3-40 membered heteroalkylene)-(Co-12 alkylene)-.

71. The compound of any one of claims 1-68, wherein the linker is C₄₋₁₄ alkylene.

72. A compound in Table 1, 2, 3, or 4, or a pharmaceutically acceptable salt thereof.

73. A compound in Table 1-A or 2-A, or a pharmaceutically acceptable salt thereof.

74. A pharmaceutical composition comprising a compound of any one of claims 1-73 and a pharmaceutically acceptable carrier.

75. A method of treating cancer, comprising administering to a patient in need thereof a therapeutically effective amount of a compound of any one of claims 1-73 to treat the cancer.

76. The method of claim 75, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

77. A method of causing death of a cancer cell, comprising contacting a cancer cell with an effective amount of a compound of any one of claims 1-73 to cause death of the cancer cell.

78. The method of claim 77, wherein the cancer cell is selected from an ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia cell.

79. A method of degrading an effector protein in a cell, comprising administering to the cell an effective amount of a compound of any one of claims 1-73, resulting in degradation of the effector protein in the cell, wherein the effector protein is GSPT1, Cyclin K, RBM23, RBM39, IKZF1, IKZF3, PLK1, CDK4, or CK1alpha.
